(19) United States
(12) Patent Application Publication
(10) Pub. No.: US 2025/0375505 A1
(43) Pub. Date: Dec. 11, 2025

(54) COMPOSITIONS AND METHODS FOR MODIFYING BILE ACIDS TO REGULATE LIPID AND STEROID METABOLISM

(71) Applicant: The Regents of the University of California, Oakland, CA (US)

(72) Inventors: Jonathan Lynch, Los Angeles, CA (US); Elaine Hsiao, Los Angeles, CA (US); Erika L. Gonzalez, Los Angeles, CA (US); Kayli Choy, Los Angeles, CA (US)

(21) Appl. No.: 18/719,756

(22) PCT Filed: Dec. 13, 2022
(86) PCT No.: PCT/US2022/052657

§ 371 Date: Jun. 13, 2024

Related U.S. Application Data
(60) Provisional application No. 63/288,980, filed on Dec. 13, 2021.
(60) Provisional application No. 63/289,412, filed on Dec. 14, 2021.
(60) Provisional application No. 63/355,381, filed on Jun. 24, 2022.

(51) Int. Cl. A61K 38/44; A61K 35/741; A61P 3/06

(52) U.S. Cl. CPC A61K 38/443; A61K 35/741; A61P 3/06; C12Y 101/01159

(57) ABSTRACT

Provided herein are methods and compositions related to treating or preventing metabolic disorder.

### CROSS-REFERENCE TO RELATED APPLICATION

This application is a U.S. National Stage Application of PCT/US22/52657, filed Dec. 13, 2022, which claims the benefit of U.S. Provisional Patent Application No. 63/288,980, filed on Dec. 13, 2021, U.S. Provisional Patent Application No. 63/289,412, filed on Dec. 14, 2021, U.S. Provisional Patent Application No. 63/355,381, filed on Jun. 24, 2022, the entire contents of these applications are incorporated herein by reference in their entirety.

### REFERENCE TO A SEQUENCE LISTING XML

This application contains a Sequence Listing which has been submitted electronically in XML format. The Sequence Listing XML is incorporated herein by reference. Said XML file, created on Dec. 9, 2024, is named UCH-31901_SL.xml and is 76,361 bytes in size.

### BACKGROUND

Metabolic disorders represent a growing worldwide health challenge due to their dramatically increasing prevalence. Metabolic disorders are associated with alterations in the composition and function of the gut microbiota. The gut microbiota can interact with the host by the production of a diverse reservoir of metabolites, from exogenous dietary substrates or endogenous host compounds. Specific classes of microbiota-derived metabolites, notably bile acids, short-chain fatty acids, branched-chain amino acids, trimethylamine N-oxide, tryptophan and indole derivatives, have been implicated in the pathogenesis of metabolic disorders. Considerable efforts have been made to understand the mechanism of metabolic disorders. Presently, it remains unclear which bacterial strains regulate bile acids and salts in the gut and by what mechanism. There remains an unmet need to identify gut bacterial strains that regulate bile acids and salts and develop microbial therapeutics to treat metabolic disorders.

### SUMMARY

Provided herein are methods and compositions for regulating bile salts or bile acids by administering compositions (e.g., the composition disclosed herein) to a subject. In certain embodiments, the methods and compositions are for the treatment or prevention of a metabolic disorder in a subject (e.g., a subject with a lipid metabolic disorder, such as hyperlipidemia, hypercholeresterolemia, acid lipase disease, Barth syndrome, Fabry disease, Farber's disease, Gaucher disease, Niemann-Pick disease, or Tay-Sachs disease; or a steroid metabolic disorder such as cytochrome p450 oxidoreductase deficiency, apparent mineralocorticoid excess, lipoid congenital adrenal hyperplasia, congenital bile acid synthesis defect, 3-beta-hydroxysteroid dehydrogenase deficiency, aldosterone-producing adenoma, polyendocrinopathy, adrenal hyperplasia).

In some aspects, provided herein are methods for preventing or treating a metabolic disorder (e.g., a lipid metabolic disorder and/or a steroid metabolic disorder) in a subject, comprising administering to the subject a composition comprising a bacterial strain (e.g., Turicibacter sanguinis, Escherichia coli, or Bacteroides thetaiotaomicron) that expresses a bile salt-regulating gene or bile acid-regulating gene.

Also provided herein are methods of treating or preventing a metabolic disorder in a subject by depleting the gut microbiota of the subject (e.g., by administering antibiotics to the subject) and administering a composition comprising a bacterial strain (e.g., Turicibacter sanguinis, Escherichia coli, or Bacteroides thetaiotaomicron) that expresses a bile salt-regulating gene or bile acid-regulating gene.

Provided herein are bacterial strains (e.g., Turicibacter sanguinis, Escherichia coli, or Bacteroides thetaiotaomicron) comprising a bile salt-regulating gene or bile acid-regulating gene, wherein the bacterial strain is genetically engineered to express the bile salt-regulating gene or bile acid-regulating gene. Similarly, provided herein are bacterial strains (e.g., Turicibacter sanguinis, Escherichia coli, or Bacteroides thetaiotaomicron) that express a bile salt-regulating gene or bile acid-regulating gene encoded by an exogenous nucleic acid, e.g., a plasmid or other vector in which the bile salt-regulating gene or bile acid-regulating gene is operably coupled to a promoter that promotes expression (e.g., constitutively or inducibly) of the bile salt-regulating gene or bile acid-regulating gene in the bacterial strain.

Also provided herein are compositions (e.g., compositions comprising bacterial strains described herein and a pharmaceutically acceptable carrier; or compositions comprising a bacterial strain (e.g., Turicibacter sanguinis, Escherichia coli, or Bacteroides thetaiotaomicron) that expresses a bile salt-regulating gene or bile acid-regulating gene (e.g., as described herein) and a pharmaceutically acceptable carrier).

The composition may be formulated for oral or rectal delivery. The composition may be self-administered. The composition may be a food or beverage product. In some embodiments, the food product is a dairy product (e.g., yogurt or kefir). In some embodiments, the composition comprises probiotics. In some embodiments, the composition comprises a fecal sample (e.g., a fecal sample from a fecal bank) comprising a bacterial straina strain (e.g., Turicibacter sanguinis, Escherichia coli, or Bacteroides thetaiotaomicron) that expresses a bile salt-regulating gene or bile acid-regulating gene.

Provided herein are methods of making a bacterial strain described herein, comprising: transforming a bacterial strain with a gene expression construct encoding a bile salt-regulating gene or bile acid-regulating gene operably coupled to a promoter that promotes expression (e.g., constitutively or inducibly) of the bile salt-regulating gene or bile acid-regulating gene in the bacterial strain. In certain embodiments, the method further comprises formulating the bacterial strain for administration to a subject, e.g., in a pharmaceutical composition or in a food or beverage product. In some embodiments, the method further comprises culturing the bacterial strain to allow expression of the bile salt-regulating gene or bile acid-regulating gene.

### BRIEF DESCRIPTION OF THE FIGURES

FIG. 1A & FIG. 1B show genomic comparison reveal distinct sub groups of Turicibacter sanguinis. A: Phylogenetic tree comparing full-length 16S rRNA sequences from noted T. sanguinis isolates. Circles indicate human-derived isolated, triangles indicate mouse-derived isolates, and squares indicate mouse-derived contaminating isolate. B: Full shotgun-assembled genome comparisons of T. sanguinis isolates listed in A.

FIG. 2A & FIG. 2B show T. sanguinis isolates differ in their bile-modifying abilities. A: Liquid chromatograms of individual T. sanguinis isolates grown for 24 hours in media with sub-inhibitory concentrations of five bile salts/acids: taurocholic acid (TCA), cholic acid (CA), glycochenodeoxycholic acid (GCDCA), chenodeoxycholic acid (CDCA), and deoxycholic acid (DCA). Shaded regions indicate expected retention time of each bile species. B: Relative amounts of remaining conjugated bile salts (tauricholic acid, taurochenodeoxycholic acid, glycocholic acid, glycochenodeoxycholic acid) after 24 growth with noted T. sanguinis isolate. Magenta=glycine-conjugated bile salts, blue-taurine-conjugated bile salts.

FIG. 3A-FIG. 3D show T. sanguinis isolates differ in their genetic capacity to modify bile species. A: Liquid chromatograms of media after 24 hour cultures of E. coli expressing individual predicted bile salt hydrolases (BSH) from each sequence grouping and isolate and grown in with TCA and TCDCA. Control is E. coli with same expression vector but expressing non-bile modifying gene. B: same as A, but with GCA and GCDCA instead of taurine-conjugated bile salts. C. Remaining amounts of TCA, TCDCA, GCA, and GCDCA after 24 hour growths of E. coli expressing noted BSH homologs. D: Phylogenetic tree of each predicted BSH gene from T. sanguinis isolates, with bile salt specificity noted in boxes. The study did not detect bile salt hydrolase activity in sequences without boxes.

FIGS. 4A-FIG. 4D show T. sanguinis BSH expression is sufficient to alter host lipidome and health-associated lipid markers. A: Heatmap of plasma lipid species significantly altered by expression of at least one T. sanguinis BSH in Bacteroides thetaiotaomicron. Colors on left correspond to lipid class, cyan-magenta colorscale represents Z-score. B. Relative white adipose tissue weight of mice monocolonized with BSH-expression B. thetatiotaomicron. C. Relative combined plasma triglycerides (TG) of mice monocolonized with BSH-expression B. thetatiotaomicron. D. Relative cholesterol esters (CE) of mice monocolonized with BSH-expression B. thetatiotaomicron. All values normalized to sex-matched littermates, *=p<0.05 two-tailed t-test. BSH3 corresponds to BSH-IV-MOL361; BSH4 corresponds to BSH-I-MOL361; BSH5 corresponds to BSH-III-1E2; BSH7 corresponds to BSH-II-H121

FIG. 5A-FIG. 5B show T. sanguinis isolates differ in their effects on host lipid biology and bile acids. A: Sex and littermatched relative abdominal fat pad mass from mice monocolonized with individual T. sanguinis isolates. B: top: Example image of adipose tissue histology section. bottom: Sex and litter matched relative adipocyte size of mice monocolonized with individual T. sanguinis isolates.

FIG. 6 shows levels of circulating serum bile acids and cholesterol in mice monocolonzed by individual T. sanguinis isolates. #=p<0.1, *=p<0.05.

FIG. 7 shows genomic comparisons reveal distinct subgroups of Turicibacter. a, Phylogenetic tree comparing full-length 16S rRNA gene sequences from noted Turicibacter isolates. Circles indicate human-derived isolates, triangles indicate mouse-derived isolates, and square indicates a mouse-derived contaminating isolate. b, Association between guanine-cytosine % (GC %) and calculated genome size in megabases (Mb) for shotgun-assembled genomes of Turicibacter isolates from a. c, Full genome sequence comparisons across Turicibacter strains. Position of predicted bile-modification gene homologs are noted outside of rings, with the color of the gene name denoting the genome family that gene is found in. Each ring represents sequence blocks in one genome. d, Average nucleotide identity (ANI) between noted Turicibacter genomes. Number denotes ANI, white to blue scale represents 100%-75% ANI scale.

FIG. 8 shows Turicibacter colonization alters host lipids in a strain-dependent manner. a, Heatmap of relative abundance of serum lipids from gnotobiotic mice monocolonized with noted Turicibacter strains. Heatmap values represent mean abundance of each detected lipid species from labeled lipid categories scaled across all the means of that individual lipid species. Black (p<0.05) and grey (p<0.1) rectangles indicate statistically significant differences of that metabolite between i) GF and MOL361 monocolonized mice, ii) CONV and MOL361 monocolonized mice, and iii) between mice colonized by different Turicibacter strains. b, Serum cholesterol concentrations of mice colonized by noted Turicibacter strains. c, Sex and litter-matched relative epidydimal/gonadal white adipose tissue (c/g WAT) mass of mice monocolonized with noted Turicibacter strains. Shapes indicate value for individual mouse, dotted bar represents combined ANOVA statistic for each group versus the experimental mean. Metabolite and cholesterol analysis n=6-10. WAT analysis n=6-26, Mann-Whitney test for MOL361-GF and MOL361-CONV comparisons, Kruskal-Wallis for intra-Turicibacter comparison. *p<0.05, ***p<0.0005.

FIG. 9 shows Turicibacter colonization alters circulating host bile species in a strain-specific manner. a-c,: Serum concentrations of a-d) primary unconjugated bile acids, e-f) secondary unconjugated bile acids, or g-l) primary conjugated bile acids, Serum levels of individual bile species from mice colonized by noted Turicibacter strains. Points indicate log-transformed value for individual mouse with shapes and colors matching FIG. 1, error bars represent mean+/−SEM. Kruskal-Wallis test across all noted colonizations with Dunn's multiple comparisons to GF for a-f. Kruskal-Wallis test between noted Turicibacter strains and multiple comparisons to H121 for g-l. Mann-Whitney test used to compare GF and MOL361 in g-l, and p-values are noted above GF data points. n=6-10 for each group. Dotted bar represents ANOVA statistic for each group versus the combined experimental mean. *p<0.05, **p<0.005, ***p<0.0005.

FIG. 10 shows Turicibacter isolates differ in their bile-modifying abilities. a, Schematic for types of bile transformations found to be performed by Turicibacter isolates. b, inset: 16S rRNA-based phylogenic tree from FIG. 7a. Liquid chromatograms of individual Turicibacter isolates grown for 24 hours in media with sub-inhibitory concentrations of five bile acids: taurocholic acid (TCA), cholic acid (CA), glycochenodeoxycholic acid (GCDCA), chenodeoxycholic acid (CDCA), and deoxycholic acid (DCA). Shaded regions indicate expected retention time of each bile species. c, Percent remaining (compared to cultures at time=0) of conjugated bile acids (TCA, taurochenodeoxycholic acid [TCDCA], glycocholic acid [GCA], GCDCA) after 24 growth with noted Turicibacter isolate. Yellow=glycine-conjugated bile acids, orange=taurine-conjugated bile acids. n=4 independent cultures. Values not shown were below 0.1% remaining. Statistical analysis performed by one sample t-test, annotations of legend denotes strains with significant difference from 100% remaining for each bile acid.

FIG. 11 shows Turicibacter isolates differ in their genetic capacity to modify bile species. a, Phylogenetic tree of amino acid sequences for each predicted bile salt hydrolase (BSH) sequence from Turicibacter strains, with observed bile species specificity noted in boxes. We did not detect bile salt hydrolase activity in sequences without boxes, representing groups V-VIII. b, Presence or absence of sequence homologs with potential BSH activity in Turicibacter strains. c, Liquid chromatograms of media after 24 hour cultures of E. coli expressing individual predicted bsh genes from each sequence grouping and grown in with TCA and TCDCA. Control is E. coli with same expression vector but expressing non-bile-modifying gene. d, same as c, but with GCA and GCDCA instead of tauro-bile acids. e, Quantification of percent remaining (compared to media controls) of conjugated bile acid (TCA, TCDCA, GCA, GCDCA) after 24 h growth with E. coli expressing the noted Turicibacter bsh gene. n=3 independent cultures, *p<0.05, **p<0.005, ***p<0.0005 using one sample t-test comparison with 100% remaining. BSH nomenclature indicates homolog group (e.g. III) and isolate of origin (e.g. MOL361).

FIG. 12 shows Turicibacter bsh expression is sufficient to alter host lipidome and health-associated lipid markers. a, Percent remaining of noted bile acids after 24 hour growth with Bacteroides thetaiotaomicron expressing noted bsh genes. n=4 cultures per strain. b, Same as a, but with 48 hour growth with noted B. thetaiotaomicron strains. n=3 cultures strain-1. For a and b, points represent individual comparison with media control, legend annotations denote strains with statistical significance for each bile acid using one sample t-test comparison with 100% remaining. c, Quantification of colonic bile acids (BA) from mice colonized with bsh-expressing B. thetaiotaomicron. Values are normalized to sex-matched littermates colonized with wild-type B. thetatiotaomicron. Statistical analysis was performed with Kruskal-Wallis test with Dunn's multiple comparisons test. n=3-4. d, Heatmap of circulating lipid species significantly altered by expression of at least one Turicibacter bsh in B. thetaiotaomicron. Colors on left correspond to lipid class, cyan-magenta color scale represents Z-score, each column represents one animal. e-i, Relative combined circulating concentrations of e, triglycerides (TG), f, cholesterol esters (CE), g, diacylglycerides (DG), h, phosphotidylglycines (PG), or i, phosphotidylserines (PS) of mice monocolonized with bsh-expressing B. thetatiotaomicron. j, Relative white adipose tissue weight of mice monocolonized with bsh-expressing B. thetatiotaomicron. All values normalized to sex-matched littermates, animal n=4-6 colonization-1, statistical analysis performed by Welch's ANOVA with Dunnet's multiple comparisons to GF, dotted bar in c-j represents ANOVA statistic for each group versus the combined experimental mean. *p<0.05, **p<0.005, ***p<0.0005.

FIG. 13 shows strain-dependent variation in colonization and adipocyte size. a-e, Representative images of adipose tissue from Turicibacter monocolonized mice. f, Sex- and litter-matched relative adipocyte area of mice monocolonized with individual Turicibacter strains. Each point represents mean of 10 images of adipocyte area per animal. g, h, Colony-forming units (CFU) equivalents per gram of contents for distal small intestine (g) or proximal colon (h). Each dot represents sample from one animal, per colonization n=10-15 for qPCR, n=10-14 for adipocyte area calculation. Statistics for adipocyte area performed by Welch's ANOVA with Dunnet's multiple comparisons, dotted bar represents ANOVA statistic for each group versus the combined experimental mean.

FIG. 14 shows host effects of Turicibacter colonization varies with sex. a-c, Heatmap of mean relative a) bile species, b) sterols, or c) lipids. Column labels represent colonization state and sex, blue-male, red=female. n=3-6 group-1, except female T129 (n=1).

FIG. 15 shows summary table of bile transformations performed by Turicibacter isolates. Table indicating presence/absence of noted bile species after growth in mixture of bile acids described in FIG. 10b.

FIG. 16 shows some strains of Turicibacter encode a functional 7alpha-HSDH gene. a, Table depicting amino acid similarity and sequence coverage between closest predicted 7alpha-HSDH homolog in noted isolate genome and 7alpha-HSDH gene from Clostridium absonum. b, Chromatograms of cholic acid (CA) or cholic acid with 2 hydrogens removed (CA-2H) from E. coli cultures expressing the MOL361 HSDH homolog (7alpha-HSDH) or non-bile modifying gene sequence in the same plasmid (cell control). Chromatograms from independent triplicate cultures are shown, dotted boxes indicate each bile species. c, Quantification of CA/CA-2H ratios determined from reconstructed areas under the curve in b. Statistical comparison performed with Welch's t-test, n=3 cultures, bars indicate mean+/−SEM. d, Same as b, but with CDCA instead of CA. e, same as b, but with DCA instead of CA. d and e triplicate chromatograms are stacked on the same axis.

FIG. 17 shows bile transformations performed by B. thetaiotaomicron expressing Turicibacter bsh genes. Table indicating presence/absence of noted bile species after growth in mixture of bile acids described in FIG. 11c-e.

FIG. 18 shows expressing bsh genes from Turicibacter does not impart significant in vitro growth defect of B. thetaiotaomicron. OD₆₀₀ readings of noted B. thetaiotaomicron strains in BHI-S medium. Each point represents mean+/−SEM for 6 independent cultures.

FIG. 19 shows sex differences in lipidomic response to Turicibacter bsh expression in B. thetaiotaomicron. Similar to FIG. 12d, but animal lipidome analysis separated into a) males and b) females. Note that lipid species presented were found to be significantly altered by expression of at least one bsh in males and females combined (i.e. all lipid species shown across the three analyses are the same). Each column represents one animal.

FIG. 20 shows Turicibacter bsh expression by B. thetaiotaomicron is sufficient to drive broad scale changes in circulating host lipids in male and female mice. Related to FIG. 12e-j, circulating concentrations of specific lipid categories in mice colonized by B. thetaiotaomicron expressing the noted Turicibacter bsh homologs. Each point represents one animal, red points indicate female, blue represent male. n=4-5 per colonization, error bars represent mean+/−SEM. Statistical comparison done with Welch's ANOVA with Dunnet's multiple comparisons, dotted bar represents combined ANOVA statistic for each group versus the experimental mean, *p<0.05.

FIG. 21 shows Turicibacter strains MOL361 and 1E2 can deconjugate at least six taurine-conjugated bile acids. Chromatograms (left) and unconjugated/conjugated bile acid ratios (right) of Turicibacter MOL361 or 1E2 grown for 24 hours in YCFA+0.5 mM of individual taurine-conjugated bile acids: a, TCA; b, TCDCA; c, tauroursodeoxycholic acid (TUDCA); d, taurolithocholic acid (TLCA); e, taurohyodeoxycholic acid (THDCA); f, taurodeoxycholic acid (TDCA). Chromatograms are concatenated reconstructed chromatograms for conjugated and unconjugated bile acid. Each trace and each point represents one biological replicate, n=3 cultures. Statistics were performed by Mann-Whitney test, #=p≤0.1 (Note: this p-value is the minimum for this test and our experimental parameters).

FIG. 22 shows bsh expression does not alter B. thetaiotaomicron colonization of the murine gut. CFU/mL quantifications of the noted B. thetaiotaomicron strains in the a, distal small intestine; b, cecum; and c, proximal colon of gnotobiotic mice.

FIG. 23 shows Turicibacter colonization and bsh expression induce similar gene expression patterns in the liver. qRT-PCR analysis of liver transcript levels of a, Fxr; b, Cyp7a1; and c, Gopase after colonization with the noted Turicibacter strains of B. thetaiotaomicron expressing the noted bsh gene. Data are displayed as fold change relative to appropriate control (GF for Turicibacter colonizations, Bt-WT for bsh colonizations). Each point represent data from a single animal, statistics performed with Kruskal-Wallis test with Dunn's multiple comparisons. Dotted horizontal line represents total ANOVA statistic for that comparison, *=p<0.05.

### DETAILED DESCRIPTION

Provided herein are methods and compositions for regulating bile salts and/or bile acids by administering compositions provided herein.

In some aspects, provided herein are methods for preventing or treating a metabolic disorder (e.g., a lipid metabolic disorder and/or a steroid metabolic disorder) in a subject, comprising administering to the subject a composition comprising a bacterial strain (e.g., Turicibacter sanguinis, Escherichia coli, or Bacteroides thetaiotaomicron) that expresses a bile salt-regulating gene or bile acid-regulating gene.

Also provided herein are methods of treating or preventing a metabolic disorder in a subject by depleting the gut microbiota of the subject (e.g., by administering antibiotics to the subject) and administering a composition comprising a bacterial strain (e.g., Turicibacter sanguinis, Escherichia coli, or Bacteroides thetaiotaomicron) that expresses a bile salt-regulating gene or bile acid-regulating gene.

Provided herein are bacterial strains (e.g., Turicibacter sanguinis, Escherichia coli, or Bacteroides thetaiotaomicron) comprising a bile salt-regulating gene or bile acid-regulating gene, wherein the bacterial strain is genetically engineered to express the bile salt-regulating gene or bile acid-regulating gene. Similarly, provided herein are bacterial strains (e.g., Turicibacter sanguinis, Escherichia coli, or Bacteroides thetaiotaomicron) that express a bile salt-regulating gene or bile acid-regulating gene encoded by an exogenous nucleic acid, e.g., a plasmid or other vector in which the bile salt-regulating gene or bile acid-regulating gene is operably coupled to a promoter that promotes expression (e.g., constitutively or inducibly) of the bile salt-regulating gene or bile acid-regulating gene in the bacterial strain.

Also provided herein are compositions (e.g., compositions comprising bacterial strains described herein and a pharmaceutically acceptable carrier; or compositions comprising a bacterial strain (e.g., Turicibacter sanguinis, Escherichia coli, or Bacteroides thetaiotaomicron) that expresses a bile salt-regulating gene or bile acid-regulating gene (e.g., as described herein) and a pharmaceutically acceptable carrier).

Provided herein are methods of making a bacterial strain described herein, comprising: transforming a bacterial strain with a gene expression construct encoding a bile salt-regulating gene or bile acid-regulating gene operably coupled to a promoter that promotes expression (e.g., constitutively or inducibly) of the bile salt-regulating gene or bile acid-regulating gene in the bacterial strain. In certain embodiments, the method further comprises formulating the bacterial strain for administration to a subject, e.g., in a pharmaceutical composition or in a food or beverage product. In some embodiments, the method further comprises culturing the bacterial strain to allow expression of the bile salt-regulating gene or bile acid-regulating gene.

In certain embodiments, the methods and compositions are for the treatment or prevention of a metabolic disorder in a subject (e.g., a subject with a lipid metabolic disorder, such as hyperlipidemia, hypercholeresterolemia, acid lipase disease, Barth syndrome, Fabry disease, Farber's disease, Gaucher disease, Niemann-Pick disease, or Tay-Sachs disease; or a steroid metabolic disorder such as cytochrome p450 oxidoreductase deficiency, apparent mineralocorticoid excess, lipoid congenital adrenal hyperplasia, congenital bile acid synthesis defect, 3-beta-hydroxysteroid dehydrogenase deficiency, aldosterone-producing adenoma, polyendocrinopathy, adrenal hyperplasia).

### Definitions

As used herein in the specification, “a” or “an” may mean one or more. As used herein in the claim(s), when used in conjunction with the word “comprising”, the words “a” or “an” may mean one or more than one. As used herein “another” may mean at least a second or more.

The phrase “pharmaceutically-acceptable carrier” as used herein means a pharmaceutically-acceptable material, composition or vehicle, such as a liquid or solid filler, diluent, excipient, solvent or encapsulating material. Each carrier must be “acceptable” in the sense of being compatible with the other ingredients of the formulation and not injurious to the subject. Some examples of materials which can serve as pharmaceutically-acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical formulations.

The term “preventing” is art-recognized, and when used in relation to a condition, such as a local recurrence, is well understood in the art, and includes administration of a composition which reduces the frequency of, or delays the onset of, symptoms of a medical condition in a subject relative to a subject which does not receive the composition.

The term “prophylactic” or “therapeutic” treatment is art-recognized and includes administration to the host of one or more of the subject compositions. If it is administered prior to clinical manifestation of the unwanted condition (e.g., disease or other unwanted state of the host animal) then the treatment is prophylactic (i.e., it protects the host against developing the unwanted condition), whereas if it is administered after manifestation of the unwanted condition, the treatment is therapeutic (i.e., it is intended to diminish, ameliorate, or stabilize the existing unwanted condition or side effects thereof).

The term “subject” refers to a mammal, including, but not limited to, a human or non-human mammal, such as a bovine, equine, canine, ovine, or feline.

A “therapeutically effective amount” of a compound with respect to the subject method of treatment refers to an amount of the compound(s) in a preparation which, when administered as part of a desired dosage regimen (to a mammal, preferably a human) alleviates a symptom, ameliorates a condition, or slows the onset of disease conditions according to clinically acceptable standards for the disorder or condition to be treated or the cosmetic purpose, e.g., at a reasonable benefit/risk ratio applicable to any medical treatment.

As used herein, the term “treating” or “treatment” includes reversing, reducing, or arresting the symptoms, clinical signs, and underlying pathology of a condition in a manner to improve or stabilize a subject's condition.

As used herein, the terms “modulate” or “modulation,” or “regulate” or “regulation” and “differentially regulated” can refer to both up regulation (i.e., activation or stimulation, e.g., by agonizing or potentiating) and down regulation (i.e., inhibition or suppression, e.g., by antagonizing, decreasing or inhibiting), unless otherwise specified or clear from the context of a specific usage.

### Therapeutic Methods

The disclosure herein, relates, in part, to the discovery that different strains of Turicibacter sanguinis differentially deconjugate and dehydrogenate bile acids and differentially affect host metabolites, including fat tissue mass, lipid composition, bile acids, and tryptophan-related metabolites. Whole genomes of different strains of Turicibacter were sequenced and genes were identified that are potentially responsible for different bile acid modifications (e.g., genes encoding bile salt hydrolase or 7-alpha hydroxysteroid dehydrogenase). Additionally, strains of Escherichia coli and Bacteroides thetaiotaomicron were developed to express Turicibacter sanguinis bile-modifying genes (e.g., genes encoding bile salt hydrolase or -alpha hydroxysteroid dehydrogenase).

In some aspects, provided herein are methods of preventing or treating a metabolic disorder (e.g., a lipid metabolic disorder and/or a steroid metabolic disorder), comprising administering to the subject a composition comprising a bacterial strain (e.g., Turicibacter sanguinis, Escherichia coli, or Bacteroides thetaiotaomicron) that that expresses a bile salt-regulating gene or bile acid-regulating gene.

Also provided herein are methods of treating or preventing a metabolic disorder in a subject by depleting the gut microbiota of the subject and administering a composition comprising a bacterial strain (e.g., Turicibacter sanguinis, Escherichia coli, or Bacteroides thetaiotaomicron) that expresses a bile salt-regulating gene or bile acid-regulating gene.

In other aspects, provided herein are methods of treating or preventing a metabolic disorder in a subject by depleting the gut microbiota of the subject and administering a composition comprising a bacterial strain (e.g., Turicibacter sanguinis, Escherichia coli, or Bacteroides thetaiotaomicron) that expresses a bile salt-regulating gene or bile acid-regulating gene.

In some aspects, the methods comprise depleting the gut microbiota of the subject prior to administration with a composition described herein (e.g., by administering antibiotics to the subject).

In some embodiments, the bacterial strain expresses a bile salt-regulating gene or bile acid-regulating gene encoded by an exogenous nucleic acid, e.g., a plasmid or other vector in which the bile salt-regulating gene or bile acid-regulating gene is operably coupled to a promoter that promotes expression (e.g., constitutively or inducibly) of the bile salt-regulating gene or bile acid-regulating gene in the bacterial strain.

Provided herein are methods of making a bacterial strain described herein, comprising: transforming a bacterial strain with a gene expression construct encoding a bile salt-regulating gene or bile acid-regulating gene operably coupled to a promoter that promotes expression (e.g., constitutively or inducibly) of the bile salt-regulating gene or bile acid-regulating gene in the bacterial strain. In certain embodiments, the method further comprises formulating the bacterial strain for administration to a subject, e.g., in a pharmaceutical composition or in a food or beverage product. In some embodiments, the method further comprises culturing the bacterial strain to allow expression of the bile salt-regulating gene or bile acid-regulating gene. In some embodiments, the bacterial strain (e.g., Turicibacter sanguinis, Escherichia coli, or Bacteroides thetaiotaomicron) regulates the bile salt by glycine conjugation or taurine conjugation. In some embodiments, the bile salt-regulating gene encodes a bile salt hydrolase (BSH), such as any one of the bile salt hydrolases shown in Table 1. In some embodiments, the BSH is encoded by a nucleic acid sequence that is at least 80%, 81%, 82%, 83%, 84%, 85%, 86%, 87%, 88%, 89%, 90%, 91%, 92%, 93%, 94%, 95%, 96%, 97%, 98%, 99% identical to any one of the nucleic acid sequences in Table 1.

TABLE 1
Exemplary Bile Salt Hydrolases (BSH)

| BSH | Bacterial | |
|---|---|---|
| Type | Strain | Nucleic Acid Sequence |
| BSH | Turicibacter | ATGTGTACAGGACTTAGCTTAGTTACAAAAGATAATAAGCATTTATTTGGACGAAATTTAGA |
| Group I | sanguinis | TGTACCCGCTACATATGGACAAGCTGTTCATATTGTTCCAAGAAATTATGATTGGTTCAATAT |
| | MOL361 | TGTGAGTGATGAAACATATACGTCAAAGTATGCTTGTATTGGGATGGGAATTGTTGTGGATC |
| | | GTCTTCCCCTTCTTTTTGATGCAGTTAATGAAAAAGGATTAGCAGGTGCCGGGTTAAATTTTA |
| | | CGCATTTTGCTAAGTTTAACGAAAAAGCTGTTGATGGAAAAACGAATATTTCAGGTTCAACT |
| | | TTCTTATACTGGGCATTAAGTAATTTCTCAAATTTAGATGAATTAAAAGAAGCCTTAAAACA |
| | | GTTAATTATTACGAATATTCCAGTTAAAGAAGGGCTTCCTGTTGCAGGACTTCACTGGATGTT |
| | | TACAGATTTAAGTGGAAAAAGTATTGTCATTGAGTACATGGAAGATGGAATGCACGTATACG |
| | | ACAATCCAGTAGGAGCCTTAACAAATGATCCAACTTTCCCTTGGCATTTAACAAATCTATGC |
| | | CAATATGTAACATTAGATACAAAAACTCCAGCACCAAAACAATTTGGAGAATATGTTGCTAA |
| | | ACCATTTGGTCATGGGCTTAATATGTGTGGAATCCCAGGAGATGCTTCTCCAGCTGCACGAT |
| | | TTGTTCGAACAGTCTATTTCCGTGATACAGTTGTCGAAGCAGATGATGAAGTCAGTGGTGTTT |
| | | CAGCATTCTTCCAAGTGCTAACAGGTGTTACCGTCATAAAAGGAAGCGAAATTGATCCAAAT |
| | | GGAGATATGAATTACACCGTCTATAAATCTTGTATGTGCCAAGAATCAGGAACATACTACTA |
| | | CACAGATTATAAAAACCGTCGTATTAGTGCCGTTTGCTTATCAAAAGCCGACCTAGATGGAA |
| | | AAGAAATCATCAGCTTTGAATATCAAGGTAAACAAGATATTTTATTCCAAAACTAA (SEQ |
| | | ID NO: 1) |
| BSH Group II | Turicibacter | ATGTGTACAGGGTTAAGTTTAACTACTAAAGATGGAAAACACTTCTTTGGT |
| | sanguinis H121 | AGAAATTTAGATGTTCCCGCTTCATACGGACAATCTGTTCATATCATTCCAA |
| | | GAAATTTCAATTGGCTAAATGTCGTAAATGGTGAAACATTAGCTTCTAAAT |
| | | ATGCGTGTATTGGAATGGGAATCGTTGTAGATAATCATCCACTTCTTTTTGA |
| | | TGCAGTCAATGAAAAAGGATTAGCTGGTGGTGGATTAAACTTTACGCACTT |
| | | TGCTAAATTTAATGAAAATGCCATCGATGGAAAAATAAATATCTCCGCATC |
| | | TGACTTTGTATACTGGGCACTTAGTAACTTTGCGGATTTAAATGAGTTACGT |
| | | CAAGCGTTAGAAAATTTAGTACTGACTAATATTCCATTTGAGCATGAGCTT |
| | | CCAGTTGCAGGTCTTCACTGGATGTTCACTGATTTATCTGGGCGTAGTATCG |
| | | TGATTGAAAATATGGCCGACGGTATGCATATTCATGAAAATCCAGTTGGTG |
| | | TTTTAACGAATGACCCAACTTTTGATTGGCACTTAACTAATTTAAGACAGTA |
| | | TGTTACCCTTGAAAAAGCAACACCGGCTCCAAAACAAATGGGAGATTTACT |
| | | GCTTCATCCATATGGTCATGGTTTAGGAATGTGTGGAATTCCAGGAGATGC |
| | | TTCACCTGCTGCACGATTTGTTAGAACGGTCTTCTTTAGAGATTCAATCGTT |
| | | GAAGCGAATGATGAAATCAGTGGCGTTACTGCTTTCCTCAACGTGTTAACA |
| | | GGGGTAACAGTTATTAAAGGGTCTGAAGTTGATCCCGATGGCTCAATGAAT |
| | | TATACCGTGTATAAATCAGCGATGTGTCAAGAAAGTGGAACTTACTATTAT |
| | | ACCGATTATTACAATCGACGTATCAATGCCGTTAAATTATCAAATGCTAAT |
| | | TTAGACTCAAAAGAAATTACAAGTTTCCCTTATCAAGGAGAGCAAGATATC |
| | | TTGTTCCAAAACTAA (SEQ ID NO: 2) |
| BSH Group III | Turicibacter | ATGTGCACAGGATTATGCTTATCTACAAAGGATGGAAAACATCTTTTTGGA |
| | sanguinis 1E2 | CGAAACCTTGATGTTCCTGCTTCTTATAACCAGGCCGTTCAAATTGTTCCAA |
| | | GGAATTTTAAGTGGCTAAACGTTGCGACCCAAGAAACAATCACCTCTAAAT |
| | | ACGCTTGCATAGCCATGGGAATCGTCATTGACAATCACCCTCTTCTCTTTGA |
| | | CGGGGTTAATGAAAAGGGATTAGCTGGTGGAGGATTAAATTTCACACACTT |
| | | CGCAAAATTCTCTTCAACTGCTGTAAAAGATAAAATCTCTTTATCAGCTTCT |
| | | GATTTCGTCTATTGGGTGCTGAGTACGTTTTCATCTTTAAGCGAGTTAAAAG |
| | | AGACCTTACCCTCTGTTATCTTAACAAGTATTCCCTTCAAACCGGATTTACC |
| | | AGTTGCGGGATTACATTGGATTTTTACAGATAAAACAGGTGAAAGTATTGT |
| | | TATTGAATATATGGAAGACGGAATGCATATCCACGATAATCCAGTCGGTGT |
| | | TCTAACAAACGACCCAACCTTCGACTGGCAATTAACAAATTTAAGCCAATA |
| | | TGTAACCCTTTCTTGTAAAACTCCACAACCAGAAGAAATGGGGAATCTACT |
| | | CGTTAAACCATTTGGTCATGGACTTGGCATGTGCGGAATTCCAGGAGACGG |
| | | TTCTCCCGCTGCAAGATTTGTCCGCACCGTCTTTTTCCGTGATGCCGTTGTT |
| | | GGAGCCGACGATGAAATAAGTGGGGTTACAGCCTTCTTTAATGTTCTATCA |
| | | GAAGTTACGGTGATGAAAGGATCTGAAATTGACCCCGATGGATCCATGAA |
| | | CTTTACCGTTTATAAATCAGCCATGTGTCAAGAATCTCAAACTTATTACTAC |
| | | ACAGATTACTATAACCGTCGAATCAACGCGGTTAAATTAACACCTGATACA |
| | | ATGAATGCTGACCACATTACAACGTTCCCTTACCTAGGAAAACAAGATATT |
| | | TGTTATCAAAATTAA (SEQ ID NO: 3) |
| BSH Group IV | Turicibacter | ATGTGTACAGCATTATCATTAACAGCAAAAGATGGTTCACATTTATTCGGT |
| | sanguinis | AGAAATATGGATATTGAATATTCATTTAATCAATCAATTTTATTAACACCAC |
| | MOL361 | GTCGTTTTGATTATAAAAATCGTGCAACAGGTGAAATGAATCAAACAAAGT |
| | | ATGCGATTATTGGAATGGGAACCATTATTGATGAACATCCATGTTATGCAG |
| | | AGCTTTTTAATGAAAAAGGATTAGCGGCAGCAGGATTAAACTTCCCTAACT |
| | | ACGCACACTGGGATGAAAAAGCAATCGAAGGAAAAACAAATATCCCTCCT |
| | | TATGATTTAGTTTTATGGGTCACTTCAAATTTTGAAACAGTTAAAGAAGTA |
| | | AAAGAAGCTTTAAAAGATGTTGTTTTAGTTGATGTTCCAGTCAATGAACAA |
| | | ACTCCAATTGCTCCTTTACACTGGATGATTTGTGATAAAACAGGTGAAAGT |
| | | ATCGTTGTAGAAAAAACAGTAAATGGTTTAAGTGTTATGGATAATAAAGTA |
| | | GGAGTTTTAACGAATGCGCCAACTTTTGATTGGCATTTAACAAACTTAACT |
| | | CAATACATGGGATTAACGTCGACACAACCAAAAGATATAACACTCGGAGA |
| | | ACAAGAATTACATCCATTAGGGCAAGGGCTTGGTGCTTTCTCATTACCAGG |
| | | AGATTATTCATCACCATCACGATTTGTTAAAGCTGCTTTCTTACGTAATAAT |
| | | ATCGATTATGCAAATGTTAATTACTCTGGAATCAGTGAATTCTTCCACATCT |
| | | TAAACGGAGTTGCAATGGTTCGAGGATCAGTCGTTACTCCTCAACATTTAA |
| | | ATGACATTACGTTATATACATCTTGTATGGATCAAGAACGTGGAATTTATT |
| | | ATTACAATACTTATACAAATCATACGATTTCATCTATTAATATGCATAATGA |
| | | AGATTTAGACGCAAAAGAAATTAAATCATTTAAATTTAATGATGAATTTGC |
| | | TGTAAATTTACAAAATTAA (SEQ ID NO: 4) |
| BSH Group V | Turicibacter | ATGTGTACAGCTATTACATTAAAGACACAAGATGGATTGCATGCATTAGGG |
| | sanguinis | AGAAATTTAGATATTGTGGCTTTATTAGATGTTGCTGTTATTTTGATTCCGA |
| | MOL361 | GGTCATATGCATTTACTCATACTATCATGAGTCTAAAAAAGAAAAATAAAT |
| | | ACGCGATGGTTGGAATGTCTACGACGTTTGAAAATCATGTCTTATTAGTTG |
| | | ACGGGATGAATGAAAAGGGATTAGCTTGCGCTGTATTAGAGTTACCTAAAT |
| | | ATGCTTCATGGAGTAAAGCATTGGATAAAGATAAAATAAATATGGCTCCTT |
| | | ATGATTTTGTTTATTGGATTTTAGCAAATTTTCAATCTCTTGAGGAAGTAAA |
| | | AGACGGATTAAAAAATGTAAATCTAGTTAATGAGTCGCTTGAAGGGAAAG |
| | | AGGTATCAGTAGATGTTCATTGGATTGTAACTGATCGTACAGGACAGTCTA |
| | | TTGTAATAGAAAAAACGAAGGGGAATTTTCGAATTTATAATAATAAGGTGG |
| | | GAGTTTTAACTAATGCTCCAACATTCGATTGGCATCTCATAAATTTAAATCG |
| | | GTATATGAATATACAAGTGACGAATCCGCATAAGGTGAAGTGGGGACATC |
| | | AGGAGTTAAGTTTTGACTCAGAAGGTTTTGGGGGAATCGGACTACCAGGAG |
| | | ATGTTTCATCTTCATCTCGTTTTGTAAAAGCAGCTTTTCTAAGAAATCATAT |
| | | TAGAGTAGAAAAAGGAGAAGATGCTTTAATAACAAGTACTTTTCATATTTT |
| | | AAGTAATGTGGCTGTTATCAAAGGAACTGCTGTTACTTGCCACCAACAATA |
| | | TTTGAAAACGCAGTGTACTAGTTGTATGTGTTTAGAGACAGGCGTTTATTA |
| | | CTATAACACTTATAATAATAATCAAATTAATGCTATTCATTTATTTGACGAG |
| | | AATTTAGATGCCTCTGAAGTTAAAGTGTTTCCTTATCAAGATAAACTGGTA |
| | | GTACAGAAACAAAATTAA (SEQ ID NO: 5) |
| BSH Group VI | Turicibacter | GTGTGTACAGCCATTACATTAAAAACAAGCGAAAATCATCATCTTGTTGGA |
| | sanguinis H121 | CGGAATTTTGATATTCACCCAATGAATGATTTATCAGTTGCTTTAGTTCCAC |
| | | GAGAATTTGAATATGTAAATCGTGTGACGAATGAAGAAATGAAAACGAAA |
| | | TATGCAGTTTTAGGAATGGGATTATTTTATGAAAATCATATTCTTTTTTGTG |
| | | ATGGAGTAAATGAAAAAGGATTATCTTGTCTGATGTTACAATTATCTAAGT |
| | | TTTCTACTTGGAGTCATAAAATCAGGAAGGATAAGGTCAATATAGCCCCAT |
| | | ATGATGTAGCTTTTTGGGTTTTATCTAATTTTTCAACGATTTCTGAATTAAT |
| | | GGAAGGTCTTAAACAGTTAAATATTGTGGCTCTTCCTGATGATCAAACGGC |
| | | ATTATCGACAGAAATTCATTGGCTTGTTAGTGATACGAGTGGTCAATCGAT |
| | | CGTCATTGAGCGAACAAGAGATAAGTTAACAGTTTATAATAATAAGGTAG |
| | | GCGTTTTAGCAAATTCTCCAACCTTTGATTGGCATTTAAATAACTTAGATTG |
| | | TTATATTAATGTCAAATCAGAGCAGCCAGAGGAAACTAAATGGGGACAAC |
| | | AAATGTTATCGCCTTATTCTAATGGCTTTGGTACGATTGGACTTCCTGGCGA |
| | | TTTTTCTTCTCCGTCACGATTTGTAAAGGCTGCTTTTTTACGAAATCATGTG |
| | | AACGTTGGGGAAGGTGATGAATCAGCAATTAGTGAATGCTTTCATATACTA |
| | | GATAACTTTGTTGTACCACGTGGGGTGGTTGAGACTCCAAAAAGAAAAGA |
| | | GTGTCATTTGACTAAGTATAGTGCATGTCTTTGTTTAGAGACTCAATTATAT |
| | | TATTATAAGACCAGCAGTAATCAGCAAATTCAAGTCATTGATTTAAACAAA |
| | | GAAAATTTAGATGCTAAAGGATTAAAACTATTTCCTTATCCAACAAGATTA |
| | | ACAGTCCATGATCAGAATTAG (SEQ ID NO: 6) |
| BSH Group VII | Turicibacter | ATGTGTACAGCAATCTCAATCAAGCATCAAACAGACCACGCATTTTTAGCA |
| | sanguinis 1E2 | AGAAACTTTGATTATGATTCGACCAGTCAATTAAAGTTAGCTAAAATTCCA |
| | | AGAAATTATAGCTCCCCGCTTGAACCAACGCTCAACACCTTTAAAACCCGA |
| | | TATTCGATCATTGGCATGATGCTATGGCATGAAGGAACCCATGTCCTAGTA |
| | | GATGGCATGAATGAAAAGGGGCTATCAGGTGCCATTTTAAATTTACCTGAC |
| | | GCCTGCGTGTGGAATAAAGCCCTCATTTCCGATGGCATCAATCTCTTACCTA |
| | | CCGATGTCTCCTTCTATTGTTTATCGCAGTTTTCTTCTGTCACCGAATTAAA |
| | | GGCGGCCATTCATCAACTCAATATTGTGGCCCCCGAGGATCATCCCTTTGCT |
| | | AAAACCACTCAAATTCACTGGATGTTTTGCGATAAAACTGGTGAGTCAATC |
| | | GTCGTCGAACAAACGGAATCAGGCCTATGTATTTATGATAACCCTATTGGG |
| | | GTTCTAACTAATGGGCCAACCTTCGATCAACAATTAATCAACATTTCCCCCT |
| | | ATCTCAAAGATAGCCAAACGGCTCCTCCCCTCCCAGGAGATGATTCCTCTC |
| | | CCTCACGCTTTATCCGGGCGGGGTATTTAAAACACCATCTTCAATGGACTC |
| | | AACAGCCGCTATCGACGGTAACTCATTGCTTTCACATTCTAGGAAATGTCG |
| | | CCCTCCTTCCTGGGATTTTAAAGCAAACACAAGGAGAAGAGTTTGAGACGC |
| | | GTTATACAGCTTGTATGGACCTTAAACACTTGCGCTATTACCTGAAATGGT |
| | | ATCATCATCTAAAGACTCAAATCATTGACTTAAAACAAGAGCAGGATGAA |
| | | AGCAAGACGCTGACTTTTTTTGAGTAA (SEQ ID NO: 7) |
| BSH Group VIII | Turicibacter | GTGAAGGAAGGAACCATCCCCTTACAACCTATCTTTGCCGTTACTTACTTCT |
| | sanguinis | TAAGTATGTGTGCTACCGTCGAAGAAGTCATTTCAAAATTAGAAAATGATG |
| | MOL361 | TTACACTTATCGCAAAGCCTGTCTTCGGGGATATCCGTAATACTCACTGGAT |
| | | GTTTAGTGACCGTACAGGAGAAACCATCATCATTGAACCTGATGTAGATAA |
| | | ACTGAAGATTCACCGACATTCAATGGGCGTCTTAACAAATAGTCCAAATTA |
| | | CGATTGGCATCGTACCAATTTATTAAATTACTGCAATATACGAAGTCTCGA |
| | | TTATTCAAGCGTTACACTGAATGACGATACGATTGAAGCCTGTTTCTCAGG |
| | | GAGCGGAGCAGCTGGACTGCCAGGAGATTTTAGCTCGCCATCTCGATTTAC |
| | | ACGTTTAGCCTTCCTCAAAAATTACGCCTGCAAAGGAAAAAATGAAACCGA |
| | | AGCTGTTACCTATATGTTCCAAACATTTAAAAACGTTCAATTTCCAATGGG |
| | | GATCGTTGAAGTTGGCGAAGATAAAACCATTACGGAGCATGATAGTGGCG |
| | | TCGTTTTATTTGATTACACGATTTACACTGCCGTTATGTGCTCTGAATCTCTT |
| | | CGCTATTACTGGGTAAGCTACCAAAACATGCGTATTCAATGTGTGGATATG |
| | | AACCCATTAATTGAGAAAAAACAAGCTGTTCAATTCGAATTAAATCCTATA |
| | | AATGACATCAAATATCTTAATTAA (SEQ ID NO: 8) |

In certain embodiments, the bile acid-regulating gene encodes a 7-alpha hydroxysteroid dehydrogenase, such as a 7-alpha hydroxysteroid dehydrogenase encoded by the exemplary nucleic acid sequence shown below. In some embodiments, the 7-alpha hydroxysteroid dehydrogenase is encoded by a nucleic acid sequence that is at least 80%, 81%, 82%, 83%, 84%, 85%, 86%, 87%, 88%, 89%, 90%, 91%, 92%, 93%, 94%, 95%, 96%, 97%, 98%, 99% identical to the exemplary 7-alpha hydroxysteroid dehydrogenase nucleic acid sequence shown below.

### Exemplary 7-Alpha Hydroxysteroid Dehydrogenase Nucleic Acid Sequence

>MOL361_HSDH (locus tag: HLK68_RS02750)
(SEQ ID NO: 9)
ATGCGAAAATTAGAGAATGCAATAGCTCTTGTCACGTCTTCAACAAGAGG
TATTGGGTTAGCGTGTGCTAAAAAATTAGCGAGTGAGGGTGCCATTGTTT
ATATGGGAGTTCGTCGTTTAGAGGTCACTCAAGAAATTTGTGATGAAGTG
GCTAAAGAGGGTTTGAAAATGAAGCCTGTCTTTTTTGACGCCTACAACAT
TGATTCTTATGAAACGATGGTAGAAGAAGTGATTAGGGAACAAGGTAAGA
TTGATATTTTAGTTAATAATTTTGGAACTGGAAGACCGGAAGTAGATTTA
GATTTAGTTAGTGGAGATGAGAAAGCTTTTTTTGATATTTTAGAAGCGAA
TATCGGATCAGTTTATCGTATCTCTAAACTGGTGATTCCTCATATGATTA
AACAAGGAAAAGGAAGCATTGTTAATATTTCATCGATTGGTGGAACGGTC
CCAGATATTTCTCGTATTGGTTATGGTGTTTCAAAAGCAGGCGTTAACAA
TATAACTCAACAAATCGCTATGCAGTATGCGCGCTATAATATAAGATGTA
ATGCGGTATTACCAGGTTTAACAGCAACCGATGCAGCTCTTGATAATATG
CCAGAACAATTTATTAAGTCTTTCTTATCGCATGTTCCTTTAAATCGCAT
GGGAACACCTGAAGACATGGCGAATGCGGTTCTATTTTTCGCTAGCGATG
ATTCTTCTTATGTAACGGGGGATATTATGGAGGTATCAGGTGGTTATCAT
TTAGGAACACCACAATATGCTGATTTTGTTGGTCGTAAAGTAGTTGAGGA
AAAG

In some embodiments, the 7-alpha hydroxysteroid dehydrogenase is at least 40%, at least 41%, at least 42%, at least 43%, at least 44%, at least 45%, at least 46%, at least 47%, at least 48%, at least 49%, at least 50%, at least 51%, at least 52%, at least 53%, at least 54%, at least 55%, at least 56%, at least 57%, at least 58%, at least 59%, at least 60%, at least 61%, at least 62%, at least 63%, at least 64%, at least 65%, at least 66%, at least 67%, at least 68%, at least 69%, or at least 70% homologous to an amino acid sequence of a Clostridium absonum 7-alpha hydroxysteroid dehydrogenase.

In certain embodiments, the bile salt or bile acid is any one of the bile salts or bile acids listed in Table 3. In some embodiments, administration of the composition increases a bile acid in the subject.

TABLE 3
Exemplary Bile Salts and Bile Acids

| Bile Salt or Bile Acid | Chemical Structure |
|---|---|
| Taurocholic acid (TCA) | 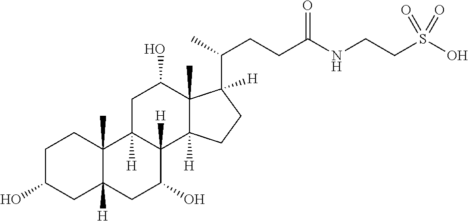 |
| Cholic acid (CA) | 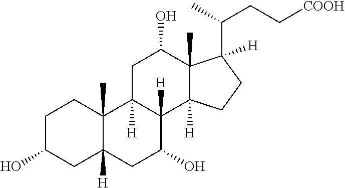 |
| Glycochenodeoxycholic acid (GCDCA) | 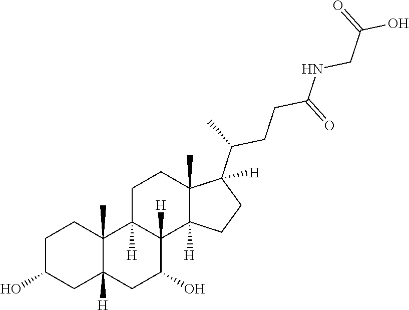 |
| Chenodeoxycholic acid (CDCA) | 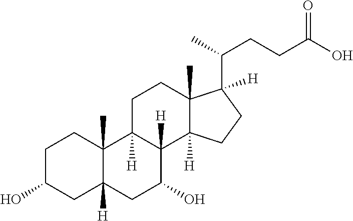 |
| Deoxycholic acid (DCA) | 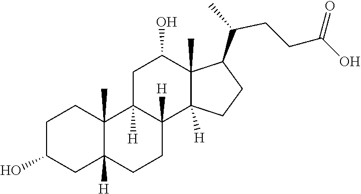 |
| Glycocholic acid | 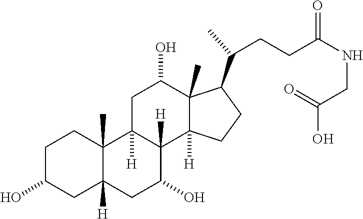 |
| 7-keto deoxycholic acid | 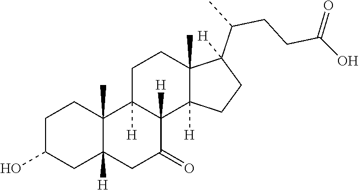 |
| Taurochenodeoxycholate | 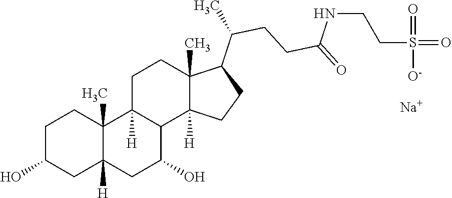 |
| Tauro-beta-muricholic acid | 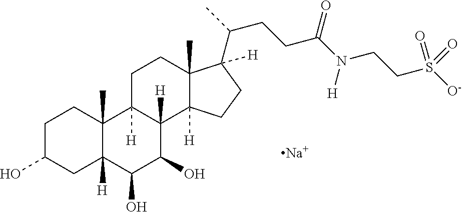 |
| Beta-muricholic acid | 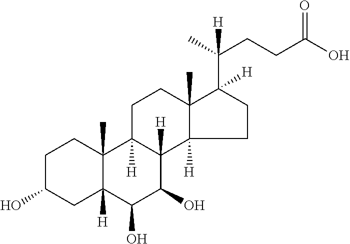 |
| Hyocholic acid | 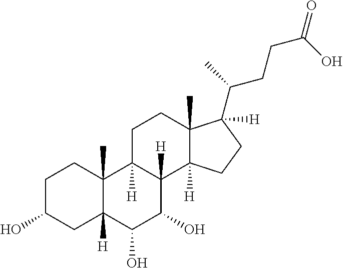 |
| 3-dehydrocholic acid | 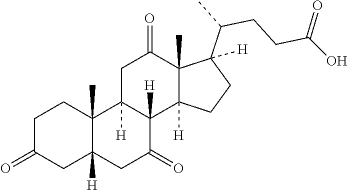 |
| Tauroursodeoxycholic | 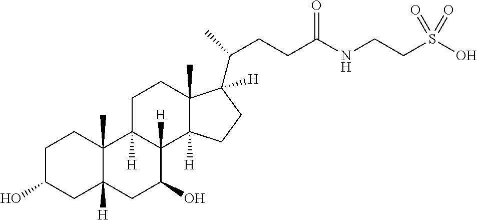 |
| Ursodeoxycholic acid | 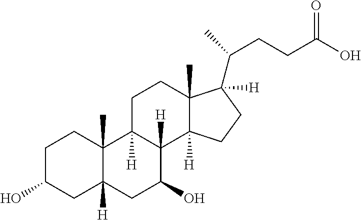 |

In certain embodiments, administration of the composition alters the subject's lipidome.

In some embodiments, administration of the composition decreases white adipose tissue weight in the subject.

In certain embodiments, administration of the composition alters health-associated lipid biomarkers in the subject (e.g., decreases triglycerides (TG) levels in the subject, decreases cholesterol levels and/or cholesterol ester (CE) levels in the subject).

In some embodiments, administration of the composition decreases abdominal fat pad mass in the subject.

In some embodiments, the bacterial strain is Turicibacter sanguinis, Escherichia coli, or Bacteroides thetaiotaomicron.

In certain embodiments, the T. sanguinis bacterial strain is any one of the T. sanguinis bacterial strains listed in Table 4.

TABLE 4
Exemplary T. sanguinis bacterial strains
Isolate of T. saunginis

MOL361
18F6
T46
GALT-E2
H121
T129
GALT-G1
1E2
TA25

In some embodiments, the T. sanguinis bacterial strain comprises a 16S nucleic acid sequence that is at least 80%, 81%, 82%, 83%, 84%, 85%, 86%, 87%, 88%, 89%, 90%, 91%, 92%, 93%, 94%, 95%, 96%, 97%, 98%, 99% identical to any one of the exemplary Turicibacter sanguinis strain 16S nucleic acid sequences shown below.

Exemplary Turicibacter sanguinis strain 16S nucleic
acid sequences:
>MOL361
(SEQ ID NO: 10)
AGAGTTTGATCCTGGCTCAGGATGAACGCTGGCGGCGTGCCTAATACATGCAAGTCGAGC
GAACCACTTCGGTGGTGAGCGGCGAACGGGTGAGTAACACGTAGGTTATCTGCCCATCAG
ACGGGGACAACGATTGGAAACGATCGCTAATACCGGATAGGACGAAAGTTTAAAGGTGCT
TCGGCACCACTGATGGATGAGCCTGCGGCGCATTAGCTAGTTGGTAGGGTAAAGGCCTAC
CAAGGCGACGATGCGTAGCCGACCTGAGAGGGTGAACGGCCACACTGGGACTGAGACACG
GCCCAGACTCCTACGGGAGGCAGCAGTAGGGAATCTTCGGCAATGGGCGAAAGCCTGACC
GAGCAACGCCGCGTGAATGATGAAGGCCTTCGGGTTGTAAAATTCTGTTATAAGGGAAGA
ATGGCTCTAGTAGGAAATGGCTAGAGTGTGACGGTACCTTATGAGAAAGCCACGGCTAAC
TACGTGCCAGCAGCCGCGGTAATACGTAGGTGGCGAGCGTTATCCGGAATTATTGGGCGT
AAAGAGCGCGCAGGTGGTTGATTAAGTCTGATGTGAAAGCCCACGGCTTAACCGTGGAGG
GTCATTGGAAACTGGTCAACTTGAGTGCAGAAGAGGGAAGTGGAATTCCATGTGTAGCGG
TGAAATGCGTAGAGATATGGAGGAACACCAGTGGCGAAGGCGGCTTCCTGGTCTGTAACT
GACACTGAGGCGCGAAAGCGTGGGGAGCAAACAGGATTAGATACCCTGGTAGTCCACGCC
GTAAACGATGAGTGCTAAGTGTTGGGGGTCGAACCTCAGTGCTGAAGTTAACGCATTAAG
CACTCCGCCTGGGGAGTACGGTCGCAAGACTGAAACTCAAAGGAATTGACGGGGACCCGC
ACAAGCGGTGGAGCATGTGGTTTAATTCGAAGCAACGCGAAGAACCTTACCAGGTCTTGA
CATACCAGTGACCGTCCTAGAGATAGGATTTTCCCTTCGGGGACAATGGATACAGGTGGT
GCATGGTTGTCGTCAGCTCGTGTCGTGAGATGTTGGGTTAAGTCCCGCAACGAGCGCAAC
CCCTGTCGTTAGTTGCCAGCATTCAGTTGGGGACTCTAACGAGACTGCCAGTGACAAACT
GGAGGAAGGTGGGGATGACGTCAAATCATCATGCCCCTTATGACCTGGGCTACACACGTG
CTACAATGGTTGGTACAAAGAGAAGCGAAGCGGTGACGTGGAGCAAACCTCATAAAGCCA
ATCTCAGTTCGGATTGTAGGCTGCAACTCGCCTACATGAAGTTGGAATCGCTAGTAATCG
CGAATCAGCATGTCGCGGTGAATACGTTCCCGGGTCTTGTACACACCGCCCGTCACACCA
CGAGAGTTTACAACACCCGAAGTCAGTGGCCTAACCGCAAGGAGGGAGCTGCCTAAGGTG
GGGTAGATGATTGGGGTGAAGTCGTAACAAGGTATCCCTACCGGAAGGTGGGGATGGATC
ACCTCCTT
>1E2
(SEQ ID NO: 11)
AGAGTTTGATCCTGGCTCAGGATGAACGCTGGCGGCGTGCCTAATACATGCAAGTCGAGC
GAACCACTTCGGTGGGAAGCGGCGAACGGGTGAGTAACACGTAGGTGATCTGCCCATCAG
ACGGGGACAACGATTGGAAACGATCGCTAATACCGGATAGGACGAAAGTTTAAAGATGCT
CCTGGCATCACTGATGGATGAGCCTGCGGCGCATTAGCTAGTTGGTGGGGTAAAGGCCTA
CCAAGGCGACGATGCGTAGCCGACCTGAGAGGGTGAACGGCCACACTGGGACTGAGACAC
GGCCCAGACTCCTACGGGAGGCAGCAGTAGGGAATCTTCGGCAATGGGCGAAAGCCTGAC
CGAGCAACGCCGCGTGAATGAAGAAGGCCTTCGGGTTGTAAAATTCTGTTATAAGGGAAG
AAAGGTGATAGGAGGAAATGACTATCAATTGACGGTACCTTATGAGAAAGCCACGGCTAA
CTACGTGCCAGCAGCCGCGGTAATACGTAGGTGGCAAGCGTTATCCGGAATTATTGGGCG
TAAAGAGCGCGCAGGTGGTTAATTAAGTCTGATGTGAAAGCCCACGGCTTAACCGTGGAG
GGTCATTGGAAACTGGTTGACTTGAGTGCAGAAGAGGGAAGTGGAATTCCATGTGTAGCG
GTGAAATGCGTAGAGATATGGAGGAACACCAGTGGCGAAGGCGGCTTCCTGGTCTGCAAC
TGACACTGAGGCGCGAAAGCGTGGGGAGCAAACAGGATTAGATACCCTGGTAGTCCACGC
CGTAAACGATGAGTGCTAAGTGTTGGGGGTCGAACCTCAGTGCTGAAGTTAACGCATTAA
GCACTCCGCCTGGGGAGTACGGTCGCAAGACTGAAACTCAAAGGAATTGACGGGGACCCG
CACAAGCGGTGGAGCATGTGGTTTAATTCGAAGCAACGCGAAGAACCTTACCAGGTCTTG
ACATACCATTGACGCCTCTAGAGATAGAGGGTTTCCTTCGGGGACAATGGATACAGGTGG
TGCATGGTTGTCGTCAGCTCGTGTCGTGAGATGTTGGGTTAAGTCCCGCAACGAGCGCAA
CCCCTGTCGTTAGTTGCCAGCAGTGAGATGGGGACTCTAACGAGACTGCCAGTGACAAAC
TGGAGGAAGGTGGGGATGACGTCAAATCATCATGCCCCTTATGACCTGGGCTACACACGT
GCTACAATGGTTGGTACAAAGAGAAGCGAAGCGGTGACGTGGAGCAAACCTCATAAAGCC
AATCTCAGTTCGGATTGTAGGCTGCAACTCGCCTACATGAAGTTGGAATCGCTAGTAATC
GCGAATCAGAATGTCGCGGTGAATACGTTCCCGGGTCTTGTACACACCGCCCGTCACACC
ACGAGAGTTTACAACACCCGAAGTCAGTGGCCTAACCGCAAGGAGGGAGCTGCCTAAGGT
GGGGTAGATGATTGGGGTGAAGTCGTAACAAGGTATCCCTACCGGAAGGTGGGGATGGAT
CACCTCCTT
>18F6
(SEQ ID NO: 12)
AGAGTTTGATCCTGGCTCAGGATGAACGCTGGCGGCGTGCCTAATACATGCAAGTCGAGC
GAACCACTTCGGTGGTGAGCGGCGAACGGGTGAGTAACACGTAGGTTATCTGCCCATCAG
ACGGGGACAACGATTGGAAACGATCGCTAATACCGGATAGGACGAAAGTTTAAAGGTGCT
TCGGCACCACTGATGGATGAGCCTGCGGCGCATTAGCTAGTTGGTAGGGTAAAGGCCTAC
CAAGGCGACGATGCGTAGCCGACCTGAGAGGGTGAACGGCCACACTGGGACTGAGACACG
GCCCAGACTCCTACGGGAGGCAGCAGTAGGGAATCTTCGGCAATGGGCGAAAGCCTGACC
GAGCAACGCCGCGTGAATGATGAAGGCCTTCGGGTTGTAAAATTCTGTTATAAGGGAAGA
ATGGCTCTAGTAGGAAATGGCTAGAGTGTGACGGTACCTTATGAGAAAGCCACGGCTAAC
TACGTGCCAGCAGCCGCGGTAATACGTAGGTGGCGAGCGTTATCCGGAATTATTGGGCGT
AAAGAGCGCGCAGGTGGTTGATTAAGTCTGATGTGAAAGCCCACGGCTTAACCGTGGAGG
GTCATTGGAAACTGGTCAACTTGAGTGCAGAAGAGGGAAGTGGAATTCCATGTGTAGCGG
TGAAATGCGTAGAGATATGGAGGAACACCAGTGGCGAAGGCGGCTTCCTGGTCTGTAACT
GACACTGAGGCGCGAAAGCGTGGGGAGCAAACAGGATTAGATACCCTGGTAGTCCACGCC
GTAAACGATGAGTGCTAAGTGTTGGGGGTCGAACCTCAGTGCTGAAGTTAACGCATTAAG
CACTCCGCCTGGGGAGTACGGTCGCAAGACTGAAACTCAAAGGAATTGACGGGGACCCGC
ACAAGCGGTGGAGCATGTGGTTTAATTCGAAGCAACGCGAAGAACCTTACCAGGTCTTGA
CATACCAGTGACCGTCCTAGAGATAGGATTTTCCCTTCGGGGACAATGGATACAGGTGGT
GCATGGTTGTCGTCAGCTCGTGTCGTGAGATGTTGGGTTAAGTCCCGCAACGAGCGCAAC
CCCTGTCGTTAGTTGCCAGCATTCAGTTGGGGACTCTAACGAGACTGCCAGTGACAAACT
GGAGGAAGGTGGGGATGACGTCAAATCATCATGCCCCTTATGACCTGGGCTACACACGTG
CTACAATGGTTGGTACAAAGAGAAGCGAAGCGGTGACGTGGAGCAAACCTCATAAAGCCA
ATCTCAGTTCGGATTGTAGGCTGCAACTCGCCTACATGAAGTTGGAATCGCTAGTAATCG
CGAATCAGCATGTCGCGGTGAATACGTTCCCGGGTCTTGTACACACCGCCCGTCACACCA
CGAGAGTTTACAACACCCGAAGTCAGTGGCCTAACCGCAAGGAGGGAGCTGCCTAAGGTG
GGGTAGATGATTGGGGTGAAGTCGTAACAAGGTATCCCTACCGGAAGGTGGGGATGGATC
ACCTCCTT
>H121
(SEQ ID NO: 13)
AGAGTTTGATCCTGGCTCAGGATGAACGCTGGCGGCGTGCCTAATACATGCAAGTCGAGC
GAACCACTTCGGTGGTGAGCGGCGAACGGGTGAGTAACACGTAGGTGATCTGCCCATCAG
ACGGGGACAACGATTGGAAACGATCGCTAATACCGGATAGGACGAAAGTTTAAAGGTGCT
TCTGGCACCGCTGATGGATGAGCCTGCGGCGCATTAGCTAGTTGGTAGGGTAAAGGCCTA
CCAAGGCGACGATGCGTAGCCGACCTGAGAGGGTGAACGGCCACACTGGGACTGAGACAC
GGCCCAGACTCCTACGGGAGGCAGCAGTAGGGAATCTTCGGCAATGGGCGAAAGCCTGAC
CGAGCAACGCCGCGTGAATGATGAAGGCCTTCGGGTTGTAAAATTCTGTTATAAGGGAAG
AACGACTTTAGTAGGAAATGGCTAGAGTGTGACGGTACCTTATGAGAAAGCCACGGCTAA
CTACGTGCCAGCAGCCGCGGTAATACGTAGGTGGCGAGCGTTATCCGGAATTATTGGGCG
TAAAGAGCGCGCAGGTGGTTGATTAAGTCTGATGTGAAAGCCCACGGCTTAACCGTGGAG
GGTCATTGGAAACTGGTCGACTTGAGTGCAGAAGAGGGAAGTGGAATTCCATGTGTAGCG
GTGAAATGCGTAGAGATATGGAGGAACACCAGTGGCGAAGGCGGCTTCCTGGTCTGTAAC
TGACACTGAGGCGCGAAAGCGTGGGGAGCAAACAGGATTAGATACCCTGGTAGTCCACGC
CGTAAACGATGAGTGCTAAGTGTTGGGGGTCGAACCTCAGTGCTGAAGTTAACGCATTAA
GCACTCCGCCTGGGGAGTACGGTCGCAAGACTGAAACTCAAAGGAATTGACGGGGACCCG
CACAAGCGGTGGAGCATGTGGTTTAATTCGAAGCAACGCGAAGAACCTTACCAGGTCTTG
ACATACCATTGACCGTTCTAGAGATAGGATTTTCCCTTCGGGGACAATGGATACAGGTGG
TGCATGGTTGTCGTCAGCTCGTGTCGTGAGATGTTGGGTTAAGTCCCGCAACGAGCGCAA
CCCCTGTCGTTAGTTGCCAGCATTCAGTTGGGGACTCTAACGAGACTGCCAGTGACAAAC
TGGAGGAAGGTGGGGATGACGTCAAATCATCATGCCCCTTATGACCTGGGCTACACACGT
GCTACAATGGTTGGTACAAAGAGAAGCGAAGCGGTGACGTGGAGCAAACCTCATAAAGCC
AATCTCAGTTCGGATTGTAGGCTGCAACTCGCCTACATGAAGTTGGAATCGCTAGTAATC
GCGAATCAGAATGTCGCGGTGAATACGTTCCCGGGTCTTGTACACACCGCCCGTCACACC
ACGAGAGTTTACAACACCCGAAGTCAGTGGCCTAACCGCAAGGAGGGAGCTGCCTAAGGT
GGGGTAGATGATTGGGGTGAAGTCGTAACAAGGTATCCCTACCGGAAGGTGGGGATGGAT
CACCTCCTT
>TA25
(SEQ ID NO: 14)
AGAGTTTGATCCTGGCTCAGGATGAACGCTGGCGGCGTGCCTAATACATGCAAGTCGAGC
GAACCACTTCGGTGGGAAGCGGCGAACGGGTGAGTAACACGTAGGTGATCTGCCCATCAG
ACGGGGACAACGATTGGAAACGATCGCTAATACCGGATAGGACGAAAGTTTAAAGATGCT
CCTGGCATCACTGATGGATGAGCCTGCGGCGCATTAGCTAGTTGGTGGGGTAAAGGCCTA
CCAAGGCGACGATGCGTAGCCGACCTGAGAGGGTGAACGGCCACACTGGGACTGAGACAC
GGCCCAGACTCCTACGGGAGGCAGCAGTAGGGAATCTTCGGCAATGGGCGAAAGCCTGAC
CGAGCAACGCCGCGTGAATGAAGAAGGCCTTCGGGTTGTAAAATTCTGTTATAAGGGAAG
AAAGGTGATAGGAGGAAATGACTATCAATTGACGGTACCTTATGAGAAAGCCACGGCTAA
CTACGTGCCAGCAGCCGCGGTAATACGTAGGTGGCAAGCGTTATCCGGAATTATTGGGCG
TAAAGAGCGCGCAGGTGGTTAATTAAGTCTGATGTGAAAGCCCACGGCTTAACCGTGGAG
GGTCATTGGAAACTGGTTGACTTGAGTGCAGAAGAGGGAAGTGGAATTCCATGTGTAGCG
GTGAAATGCGTAGAGATATGGAGGAACACCAGTGGCGAAGGCGGCTTCCTGGTCTGCAAC
TGACACTGAGGCGCGAAAGCGTGGGGAGCAAACAGGATTAGATACCCTGGTAGTCCACGC
CGTAAACGATGAGTGCTAAGTGTTGGGGGTCGAACCTCAGTGCTGAAGTTAACGCATTAA
GCACTCCGCCTGGGGAGTACGGTCGCAAGACTGAAACTCAAAGGAATTGACGGGGACCCG
CACAAGCGGTGGAGCATGTGGTTTAATTCGAAGCAACGCGAAGAACCTTACCAGGTCTTG
ACATACCATTGACGCCTCTAGAGATAGAGGGTTTCCTTCGGGGACAATGGATACAGGTGG
TGCATGGTTGTCGTCAGCTCGTGTCGTGAGATGTTGGGTTAAGTCCCGCAACGAGCGCAA
CCCCTGTCGTTAGTTGCCAGCAGTAAGATGGGGACTCTAACGAGACTGCCAGTGACAAAC
TGGAGGAAGGTGGGGATGACGTCAAATCATCATGCCCCTTATGACCTGGGCTACACACGT
GCTACAATGGTTGGTACAAAGAGAAGCGAAGCGGTGACGTGGAGCAAACCTCATAAAGCC
AATCTCAGTTCGGATTGTAGGCTGCAACTCGCCTACATGAAGTTGGAATCGCTAGTAATC
GCGAATCAGAATGTCGCGGTGAATACGTTCCCGGGTCTTGTACACACCGCCCGTCACACC
ACGAGAGTTTACAACACCCGAAGTCAGTGGCCTAACCGCAAGGAGGGAGCTGCCTAAGGT
GGGGTAGATGATTGGGGTGAAGTCGTAACAAGGTATCCCTACCGGAAGGTGGGGATGGAT
CACCTCCTT
>T46
(SEQ ID NO: 15)
AGAGTTTGATCCTGGCTCAGGATGAACGCTGGCGGCGTGCCTAATACATGCAAGTCGAGC
GAACCACTTCGGTGGTGAGCGGCGAACGGGTGAGTAACACGTAGGTTATCTGCCCATCAG
ACGGGGACAACGATTGGAAACGATCGCTAATACCGGATAGGACGAAAGTTTAAAGGTGCT
TCGGCACCACTGATGGATGAGCCTGCGGCGCATTAGCTAGTTGGTAGGGTAAAGGCCTAC
CAAGGCGACGATGCGTAGCCGACCTGAGAGGGTGAACGGCCACACTGGGACTGAGACACG
GCCCAGACTCCTACGGGAGGCAGCAGTAGGGAATCTTCGGCAATGGGCGAAAGCCTGACC
GAGCAACGCCGCGTGAATGATGAAGGCCTTCGGGTTGTAAAATTCTGTTATAAGGGAAGA
ATGGCTCTAGTAGGAAATGGCTAGAGTGTGACGGTACCTTATGAGAAAGCCACGGCTAAC
TACGTGCCAGCAGCCGCGGTAATACGTAGGTGGCGAGCGTTATCCGGAATTATTGGGCGT
AAAGAGCGCGCAGGTGGTTGATTAAGTCTGATGTGAAAGCCCACGGCTTAACCGTGGAGG
GTCATTGGAAACTGGTCAACTTGAGTGCAGAAGAGGGAAGTGGAATTCCATGTGTAGCGG
TGAAATGCGTAGAGATATGGAGGAACACCAGTGGCGAAGGCGGCTTCCTGGTCTGTAACT
GACACTGAGGCGCGAAAGCGTGGGGAGCAAACAGGATTAGATACCCTGGTAGTCCACGCC
GTAAACGATGAGTGCTAAGTGTTGGGGGTCGAACCTCAGTGCTGAAGTTAACGCATTAAG
CACTCCGCCTGGGGAGTACGGTCGCAAGACTGAAACTCAAAGGAATTGACGGGGACCCGC
ACAAGCGGTGGAGCATGTGGTTTAATTCGAAGCAACGCGAAGAACCTTACCAGGTCTTGA
CATACCAGTGACCGTCCTAGAGATAGGATTTTCCCTTCGGGGACAATGGATACAGGTGGT
GCATGGTTGTCGTCAGCTCGTGTCGTGAGATGTTGGGTTAAGTCCCGCAACGAGCGCAAC
CCCTGTCGTTAGTTGCCAGCATTCAGTTGGGGACTCTAACGAGACTGCCAGTGACAAACT
GGAGGAAGGTGGGGATGACGTCAAATCATCATGCCCCTTATGACCTGGGCTACACACGTG
CTACAATGGTTGGTACAAAGAGAAGCGAAGCGGTGACGTGGAGCAAACCTCATAAAGCCA
ATCTCAGTTCGGATTGTAGGCTGCAACTCGCCTACATGAAGTTGGAATCGCTAGTAATCG
CGAATCAGCATGTCGCGGTGAATACGTTCCCGGGTCTTGTACACACCGCCCGTCACACCA
CGAGAGTTTACAACACCCGAAGTCAGTGGCCTAACCGCAAGGAGGGAGCTGCCTAAGGTG
GGGTAGATGATTGGGGTGAAGTCGTAACAAGGTATCCCTACCGGAAGGTGGGGATGGATC
ACCTCCTT
>T129
(SEQ ID NO: 16)
AGAGTTTGATCCTGGCTCAGGATGAACGCTGGCGGCGTGCCTAATACATGCAAGTCGAGC
GAACCACTTCGGTGGTGAGCGGCGAACGGGTGAGTAACACGTAGGTGATCTGCCCATCAG
ACGGGGACAACGATTGGAAACGATCGCTAATACCGGATAGGACGAAAGTTTAAAGGTGCT
TCTGGCACCGCTGATGGATGAGCCTGCGGCGCATTAGCTAGTTGGTAGGGTAAAGGCCTA
CCAAGGCGACGATGCGTAGCCGACCTGAGAGGGTGAACGGCCACACTGGGACTGAGACAC
GGCCCAGACTCCTACGGGAGGCAGCAGTAGGGAATCTTCGGCAATGGGCGAAAGCCTGAC
CGAGCAACGCCGCGTGAATGATGAAGGCCTTCGGGTTGTAAAATTCTGTTATAAGGGAAG
AACGACTTTAGTAGGAAATGGCTAGAGTGTGACGGTACCTTATGAGAAAGCCACGGCTAA
CTACGTGCCAGCAGCCGCGGTAATACGTAGGTGGCGAGCGTTATCCGGAATTATTGGGCG
TAAAGAGCGCGCAGGTGGTTGATTAAGTCTGATGTGAAAGCCCACGGCTTAACCGTGGAG
GGTCATTGGAAACTGGTCGACTTGAGTGCAGAAGAGGGAAGTGGAATTCCATGTGTAGCG
GTGAAATGCGTAGAGATATGGAGGAACACCAGTGGCGAAGGCGGCTTCCTGGTCTGTAAC
TGACACTGAGGCGCGAAAGCGTGGGGAGCAAACAGGATTAGATACCCTGGTAGTCCACGC
CGTAAACGATGAGTGCTAAGTGTTGGGGGTCGAACCTCAGTGCTGAAGTTAACGCATTAA
GCACTCCGCCTGGGGAGTACGGTCGCAAGACTGAAACTCAAAGGAATTGACGGGGACCCG
CACAAGCGGTGGAGCATGTGGTTTAATTCGAAGCAACGCGAAGAACCTTACCAGGTCTTG
ACATACCATTGACCGTTCTAGAGATAGGATTTTCCCTTCGGGGACAATGGATACAGGTGG
TGCATGGTTGTCGTCAGCTCGTGTCGTGAGATGTTGGGTTAAGTCCCGCAACGAGCGCAA
CCCCTGTCGTTAGTTGCCAGCATTCAGTTGGGGACTCTAACGAGACTGCCAGTGACAAAC
TGGAGGAAGGTGGGGATGACGTCAAATCATCATGCCCCTTATGACCTGGGCTACACACGT
GCTACAATGGTTGGTACAAAGAGAAGCGAAGCGGTGACGTGGAGCAAACCTCATAAAGCC
AATCTCAGTTCGGATTGTAGGCTGCAACTCGCCTACATGAAGTTGGAATCGCTAGTAATC
GCGAATCAGAATGTCGCGGTGAATACGTTCCCGGGTCTTGTACACACCGCCCGTCACACC
ACGAGAGTTTACAACACCCGAAGTCAGTGGCCTAACCGCAAGGAGGGAGCTGCCTAAGGT
GGGGTAGATGATTGGGGTGAAGTCGTAACAAGGTATCCCTACCGGAAGGTGGGGATGGAT
CACCTCCTT
>GALT_E2
(SEQ ID NO: 17)
AGAGTTTGATCATGGCTCAGGATGAACGCTGGCGGCGTGCCTAATACATGCAAGTCGAGC
GAACCACTTCGGTGGTGAGCGGCGAACGGGTGAGTAACACGTAGGTTATCTGCCCATCAG
ACGGGGACAACGATTGGAAACGATCGCTAATACCGGATAGGACGAAAGTTTAAAGGTGCT
TCGGCACCACTGATGGATGAGCCTGCGGCGCATTAGCTAGTTGGTAGGGTAAAGGCCTAC
CAAGGCGACGATGCGTAGCCGACCTGAGAGGGTGAACGGCCACACTGGGACTGAGACACG
GCCCAGACTCCTACGGGAGGCAGCAGTAGGGAATCTTCGGCAATGGGCGAAAGCCTGACC
GAGCAACGCCGCGTGAATGATGAAGGCCTTCGGGTTGTAAAATTCTGTTATAAGGGAAGA
ATGGCTCTAGTAGGAAATGGCTAGAGTGTGACGGTACCTTATGAGAAAGCCACGGCTAAC
TACGTGCCAGCAGCCGCGGTAATACGTAGGTGGCGAGCGTTATCCGGAATTATTGGGCGT
AAAGAGCGCGCAGGTGGTTGATTAAGTCTGATGTGAAAGCCCACGGCTTAACCGTGGAGG
GTCATTGGAAACTGGTCAACTTGAGTGCAGAAGAGGGAAGTGGAATTCCATGTGTAGCGG
TGAAATGCGTAGAGATATGGAGGAACACCAGTGGCGAAGGCGGCTTCCTGGTCTGTAACT
GACACTGAGGCGCGAAAGCGTGGGGAGCAAACAGGATTAGATACCCTGGTAGTCCACGCC
GTAAACGATGAGTGCTAAGTGTTGGGGGTCGAACCTCAGTGCTGAAGTTAACGCATTAAG
CACTCCGCCTGGGGAGTACGGTCGCAAGACTGAAACTCAAAGGAATTGACGGGGACCCGC
ACAAGCGGTGGAGCATGTGGTTTAATTCGAAGCAACGCGAAGAACCTTACCAGGTCTTGA
CATACCAGTGACCGTCCTAGAGATAGGATTTTCCCTTCGGGGACAATGGATACAGGTGGT
GCATGGTTGTCGTCAGCTCGTGTCGTGAGATGTTGGGTTAAGTCCCGCAACGAGCGCAAC
CCCTGTCGTTAGTTGCCAGCATTCAGTTGGGGACTCTAACGAGACTGCCAGTGACAAACT
GGAGGAAGGTGGGGATGACGTCAAATCATCATGCCCCTTATGACCTGGGCTACACACGTG
CTACAATGGTTGGTACAAAGAGAAGCGAAGCGGTGACGTGGAGCAAACCTCATAAAGCCA
ATCTCAGTTCGGATTGTAGGCTGCAACTCGCCTACATGAAGTTGGAATCGCTAGTAATCG
CGAATCAGCATGTCGCGGTGAATACGTTCCCGGGTCTTGTACACACCGCCCGTCACACCA
CGAGAGTTTACAACACCCGAAGTCAGTGGCCTAACCGCAAGGAGGGAGCTGCCTAAGGTG
GGGTAGATGATTGGGGTGAAGTCGTAACAAGGTA
>GALT_G1
(SEQ ID NO: 18)
AGAGTTTGATCATGGCTCAGGATGAACGCTGGCGGCGTGCCTAATACATGCAAGTCGAGC
GAACCACTTCGGTGGTGAGCGGCGAACGGGTGAGTAACACGTAGGTGATCTGCCCATCAG
ACGGGGACAACGATTGGAAACGATCGCTAATACCGGATAGGACGAAAGTTTAAAGGTGCT
TCTGGCACCGCTGATGGATGAGCCTGCGGCGCATTAGCTAGTTGGTAGGGTAAAGGCCTA
CCAAGGCGACGATGCGTAGCCGACCTGAGAGGGTGAACGGCCACACTGGGACTGAGACAC
GGCCCAGACTCCTACGGGAGGCAGCAGTAGGGAATCTTCGGCAATGGGCGAAAGCCTGAC
CGAGCAACGCCGCGTGAATGATGAAGGCCTTCGGGTTGTAAAATTCTGTTATAAGGGAAG
AACGACTTTAGTAGGAAATGGCTAGAGTGTGACGGTACCTTATGAGAAAGCCACGGCTAA
CTACGTGCCAGCAGCCGCGGTAATACGTAGGTGGCGAGCGTTATCCGGAATTATTGGGCG
TAAAGAGCGCGCAGGTGGTTGATTAAGTCTGATGTGAAAGCCCACGGCTTAACCGTGGAG
GGTCATTGGAAACTGGTCGACTTGAGTGCAGAAGAGGGAAGTGGAATTCCATGTGTAGCG
GTGAAATGCGTAGAGATATGGAGGAACACCAGTGGCGAAGGCGGCTTCCTGGTCTGTAAC
TGACACTGAGGCGCGAAAGCGTGGGGAGCAAACAGGATTAGATACCCTGGTAGTCCACGC
CGTAAACGATGAGTGCTAAGTGTTGGGGGTCGAACCTCAGTGCTGAAGTTAACGCATTAA
GCACTCCGCCTGGGGAGTACGGTCGCAAGACTGAAACTCAAAGGAATTGACGGGGACCCG
CACAAGCGGTGGAGCATGTGGTTTAATTCGAAGCAACGCGAAGAACCTTACCAGGTCTTG
ACATACCATTGACCGTTCTAGAGATAGGATTTTCCCTTCGGGGACAATGGATACAGGTGG
TGCATGGTTGTCGTCAGCTCGTGTCGTGAGATGTTGGGTTAAGTCCCGCAACGAGCGCAA
CCCCTGTCGTTAGTTGCCAGCATTCAGTTGGGGACTCTAACGAGACTGCCAGTGACAAAC
TGGAGGAAGGTGGGGATGACGTCAAATCATCATGCCCCTTATGACCTGGGCTACACACGT
GCTACAATGGTTGGTACAAAGAGAAGCGAAGCGGTGACGTGGAGCAAACCTCATAAAGCC
AATCTCAGTTCGGATTGTAGGCTGCAACTCGCCTACATGAAGTTGGAATCGCTAGTAATC
GCGAATCAGAATGTCGCGGTGAATACGTTCCCGGGTCTTGTACACACCGCCCGTCACACC
ACGAGAGTTTACAACACCCGAAGTCAGTGGCCTAACCGCAAGGAGGGAGCTGCCTAAGGT
GGGGTAGATGATTGGGGTGAAGTCGTAACAAGGTA

In some embodiments, the subject has a metabolic disorder (e.g., a lipid metabolic disorder and/or a steroid metabolic disorder). The lipid metabolic disorder may be hyperlipidemia, hypercholeresterolemia, acid lipase disease, Barth syndrome, Fabry disease, Farber's disease, Gaucher disease, Niemann-Pick disease, or Tay-Sachs disease. The steroid metabolic disorder may be cytochrome p450 oxidoreductase deficiency, apparent mineralocorticoid excess, lipoid congenital adrenal hyperplasia, congenital bile acid synthesis defect, 3-beta-hydroxysteroid dehydrogenase deficiency, aldosterone-producing adenoma, polyendocrinopathy, adrenal hyperplasia.

The composition may be formulated for oral delivery. In some embodiments, the composition may comprise probiotics. In some embodiments, the compositions disclosed herein are food products. The composition may be in the form of a pill, tablet, or capsule. In some embodiments, the subject may be a mammal (e.g., a human). In some embodiments, the composition is self-administered. While it is preferred for a single composition to comprise all the bacteria to be administered, it will be recognized that for any of the various embodiments described herein, the combination of bacteria can similarly be administered in multiple compositions that together comprise the combination of bacteria. For example, the invention further provides kits comprising multiple compositions that together that comprise a Turicibacter sanguinis bacterial strain (e.g., a bacterial strain listed in Table 4) and/or a Turicibacter sanguinis bacterial strain that regulates a bile salt and/or a bile acid (e.g., a bacterial strain listed in Table 4).

In some embodiments, the composition is formulated for rectal delivery (e.g., a fecal sample). In some embodiments, the subject undergoes fecal microbiota transplant, wherein the transplant comprises a composition disclosed herein. Fecal microbiota transplantation (FMT), also commonly known as ‘fecal bacteriotherapy’ represents a therapeutic protocol that allows the reconstitution of colon microbial communities. The process involves the transplantation of fecal bacteria from a healthy individual into a recipient. FMT restores colonic microflora by introducing healthy bacterial flora through infusion of a fecal sample, e.g., by enema, orogastric tube or by mouth in the form of a capsule containing freeze-dried material, obtained from a healthy donor. In some embodiments, the fecal sample is from a fecal bank.

In some embodiments, the bacterial DNA in subject's gut microbiota is sequenced. The subject's gut bacterial DNA may be sequenced prior to administration of the composition. For example, a sample comprising bacterial DNA may be obtained from the subject, and the bacterial DNA is then sequenced for any one of the bacteria listed in Table 4, therefore measuring the presence or level of any one of such bacteria (e.g., one or more, two or more, five or more, or ten or more of the bacteria of interest) in the subject's gut microbiota. The composition disclosed herein may then be administered to the subject if the level of the bacteria is low. In some embodiments, the subject is deemed to have low levels of any one of the bacteria listed in Table 4 if less than 0.0001%, less than 0.001%, less than 0.01%, less than 0.02%, less than 0.03%, less than 0.04%, less than 0.05%, less than 0.06% less than 0.07%, less than 0.08%, less than 0.09%, less than 0.1%, less than 0.2%, less than 0.3% less than 0.4%, less than 0.5%, less than 0.6%, less than 0.7%, less than 0.8%, less than 0.9%, less than 1%, less than 2%, less than 3%, less than 5%, less than 7%, less than 10%, less than 20%, less than 30%, less than 40%, or less than 50% of the bacteria in the sample is the bacteria of interest. Bacterial DNA to be sequenced may be obtained through any means known in the art, including, but not limited to, obtaining a fecal sample from the subject and isolating the bacterial DNA. Bacterial DNA sequencing by any known technique in the art, including, but not limited to, Maxam Gilbert sequencing, Sanger sequencing, shotgun sequencing, bridge PCR, or next generation sequencing methods, such as massively parallel signature sequencing (MPSS), polony sequencing, 454 pyrosequencing, Illumina (Solexa) sequencing, SOLID sequencing, Ion torrent semiconductor sequencing, DNA nanoball sequencing, heliscope single molecule sequencing, single molecule real time (SMRT) sequencing, or nanopore DNA sequencing.

In some embodiments, the above methods directly act to reduce the amount of pathogenic bacteria in a subject (i.e., in the gastrointestinal tract of the subject). In some embodiments, this includes any such therapy that achieves the same goal of reducing the number of pathogenic organisms, when used in combination with the compositions described herein, would lead to replacement of the pathogenic microflora involved in the diseased state with microflora associated with a non-diseased state, or less pathogenic species occupying the same ecological niche as the type causing a disease state. For example, a subject may undergo treatment with antibiotics (e.g., antimicrobial compounds) or a composition comprising antibiotics to target and decrease the prevalence of pathogenic organisms, and subsequently be treated with a composition described herein. The treatment may also comprise an antifungal or anti-viral compound.

Suitable antimicrobial compounds include capreomycins, including capreomycin IA, caprcomycin IB, caprcomycin IIA and capreomycin IIB; carbomycins, including carbomycin A; carumonam; cefaclor, cefadroxil, cefamandole, cefatrizine, cefazedone, cefazolin, cefbuperazone, cefcapene pivoxil, cefclidin, cefdinir, cefditoren, cefime, ceftamet, cefmenoxime, cefmetzole, cefminox, cefodizime, cefonicid, cefoperazone, ceforanide, cefotaxime, cefotetan, cefotiam, cefoxitin, cefpimizole, cefpiramide, cefpirome, cefprozil, cefroxadine, cefsulodin, ceftazidime, cefteram, ceftezole, ceftibuten, ceftiofur, ceftizoxime, ceftriaxone, cefuroxime, cefuzonam, cephalexin, cephalogycin, cephaloridine, cephalosporin C, cephalothin, cephapirin, cephamycins, such as cephamycin C, cephradine, chlortetracycline; chlarithromycin, clindamycin, clometocillin, clomocycline, cloxacillin, cyclacillin, danofloxacin, demeclocyclin, destomycin A, dicloxacillin, dirithromycin, doxycyclinepicillin, crythromycin A, ethanbutol, fenbenicillin, flomoxef, florfenicol, floxacillin, flumequine, fortimicin A, fortimicin B, forfomycin, foraltadone, fusidic acid, gentamycin, glyconiazide, guamecycline, hetacillin, idarubicin, imipenem, isepamicin, josamycin, kanamycin, leumycins such as leumycin Al, lincomycin, lomefloxacin, loracarbef, lymecycline, meropenam, metampicillin, methacycline, methicillin, mezlocillin, micronomicin, midecamycins such as midecamycin A1, mikamycin, minocycline, mitomycins such as mitomycin C, moxalactam, mupirocin, nafcillin, netilicin, norcardians such as norcardian A, oleandomycin, oxytetracycline, panipenam, pazufloxacin, penamecillin, penicillins such as penicillin G, penicillin N and penicillin O, penillic acid, pentylpenicillin, peplomycin, phenethicillin, pipacyclin, piperacilin, pirlimycin, pivampicillin, pivcefalexin, porfiromycin, propiallin, quinacillin, ribostamycin, rifabutin, rifamide, rifampin, rifamycin SV, rifapentine, rifaximin, ritipenem, rekitamycin, rolitetracycline, rosaramicin, roxithromycin, sancycline, sisomicin, sparfloxacin, spectinomycin, streptozocin, sulbenicillin, sultamicillin, talampicillin, teicoplanin, temocillin, tetracyclin, thostrepton, tiamulin, ticarcillin, tigemonam, tilmicosin, tobramycin, tropospectromycin, trovafloxacin, tylosin, and vancomycin, and analogs, derivatives, pharmaceutically acceptable salts, esters, prodrugs, and protected forms thereof.

Suitable anti-fungal compounds include ketoconazole, miconazole, fluconazole, clotrimazole, undecylenic acid, sertaconazole, terbinafine, butenafine, clioquinol, haloprogin, nystatin, naftifine, tolnaftate, ciclopirox, amphotericin B, or tea tree oil and analogs, derivatives, pharmaceutically acceptable salts, esters, prodrugs, and protected forms thereof.

### Compositions

In some aspects, the invention relates to a composition (e.g., a food product or a pharmaceutical composition). Provided herein are compositions (e.g., compositions comprising a Turicibacter sanguinis bacterial strain, such as any bacteria listed in Table 4, and a pharmaceutically acceptable carrier; or compositions comprising a Turicibacter sanguinis bacterial strain that regulates a bile salt and/or a bile acid, such as any bacteria listed in Table 4, and a pharmaceutically acceptable carrier). composition may comprise at least one, at least two, at least three, at least four, at least five, at least six at least seven, at least eight, at least nine, at least ten, at least fifteen, or at least twenty bacteria that regulate a bile salt and/or a bile acid, such as any bacteria listed in Table 4. Any combination of the bacteria listed in Table 4 may be include in the composition.

The composition may comprise a pharmaceutically acceptable carrier. The composition may comprise probiotics. The pharmaceutical compositions disclosed herein may be delivered by any suitable route of administration, including orally, bucally, sublingually, parenterally, and rectally, as by powders, ointments, drops, liquids, gels, tablets, capsules, pills, or creams. In certain embodiments, the pharmaceutical compositions are delivered generally (e.g., via oral administration). In certain other embodiments, the compositions disclosed herein are delivered rectally.

In certain embodiments, the invention provides kits comprising multiple compositions (e.g., compositions comprising a Turicibacter sanguinis bacterial strain, such as any bacteria listed in Table 4, and a pharmaceutically acceptable carrier; or compositions comprising a Turicibacter sanguinis bacterial strain that regulates a bile salt and/or a bile acid, such as any bacteria listed in Table 4, and a pharmaceutically acceptable carrier). The kits disclosed herein may comprise at least one, at least two, at least three, at least four, at least five, at least six at least seven, at least eight, at least nine, at least ten, at least fifteen, or at least twenty of the compounds listed in Table 1. The kits provided herein may comprise at least one, at least two, at least three, at least four, at least five, at least six at least seven, at least eight, at least nine, at least ten, at least fifteen, or at least twenty bacteria that regulate a bile salt and/or a bile acid, such as any bacteria listed in Table 4. Any combination of the bacteria listed in Table 4 may be include in the composition.

In some embodiments, at least 1%, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or at least 95%, of the bacteria in the composition that regulate a bile salt and/or a bile acid, such as any bacteria listed in Table 4.

Compositions described herein may be used for oral administration to the gastrointestinal tract, directed at the objective of introducing the bacteria (e.g., the bacteria disclosed herein) to tissues of the gastrointestinal tract. The formulation for a composition (e.g., a probiotic composition) of the present invention may also include other probiotic agents or nutrients which promote spore germination and/or bacterial growth. An exemplary material is a bifidogenic oligosaccharide, which promotes the growth of beneficial probiotic bacteria. In some embodiments, the probiotic bacterial composition is administered with a therapeutically-effective dose of an (preferably, broad spectrum) antibiotic, or an anti-fungal agent. In some embodiments, the compositions described herein are encapsulated into an enterically-coated, time-released capsule or tablet. The enteric coating allows the capsule/tablet to remain intact (i.e., undissolved) as it passes through the gastrointestinal tract, until after a certain time and/or until it reaches a certain part of the GI tract (e.g., the small intestine). The time-released component prevents the “release” of the probiotic bacterial strain in the compositions described herein for a pre-determined time period.

The composition may be a food product, such as, but not limited to, a dairy product. The dairy product may be cultured or a non-cultured (e.g., milk) dairy product. Non-limiting examples of cultured dairy products include yogurt, cottage cheese, sour cream, kefir, buttermilk, etc. Dairy products also often contain various specialty dairy ingredients, e.g. whey, non-fat dry milk, whey protein concentrate solids, etc. The dairy product may be processed in any way known in the art to achieve desirable qualities such as flavor, thickening power, nutrition, specific microorganisms and other properties such as mold growth control. The compositions of the present invention may also include known antioxidants, buffering agents, and other agents such as coloring agents, flavorings, vitamins, or minerals.

In some embodiments, the compositions of the present invention are combined with a carrier (e.g., a pharmaceutically acceptable carrier) which is physiologically compatible with the gastrointestinal tissue of the subject(s) to which it is administered. Carriers can be comprised of solid-based, dry materials for formulation into tablet, capsule or powdered form; or the carrier can be comprised of liquid or gel-based materials for formulations into liquid or gel forms. The specific type of carrier, as well as the final formulation depends, in part, upon the selected route(s) of administration. The therapeutic composition of the present invention may also include a variety of carriers and/or binders. In some embodiments, the carrier is micro-crystalline cellulose (MCC) added in an amount sufficient to complete the one gram dosage total weight. Carriers can be solid-based dry materials for formulations in tablet, capsule or powdered form, and can be liquid or gel-based materials for formulations in liquid or gel forms, which forms depend, in part, upon the routes of administration. Typical carriers for dry formulations include, but are not limited to: trehalose, malto-dextrin, rice flour, microcrystalline cellulose (MCC) magnesium sterate, inositol, FOS, GOS, dextrose, sucrose, and like carriers. Suitable liquid or gel-based carriers include but are not limited to: water and physiological salt solutions; urea; alcohols and derivatives (e.g., methanol, ethanol, propanol, butanol); glycols (e.g., ethylene glycol, propylene glycol, and the like). Preferably, water-based carriers possess a neutral pH value (i.e., pH 7.0). Other carriers or agents for administering the compositions described herein are known in the art, e.g., in U.S. Pat. No. 6,461,607.

In some embodiments, the composition further comprises other bacteria or microorganisms known to colonize the gastrointestinal tract. For example, the composition may comprise species belonging to the Firmicutes phylum, the Proteobacteria phylum, the Tenericutes phylum, the Actinobacteria phylum, or a combination thereof. Examples of additional bacteria and microorganisms that may be included in the subject compositions include, but are not limited to, Saccharomyces, Bacteroides, Eubacterium, Clostridium, Lactobacillus, Fusobacterium, Propionibacterium, Streptococcus, Enteroccus, Lactococcus and Staphylococcus, Peptostreptococcus. In certain embodiments, the composition is substantially free of bacteria that increase the risk of metabolic disorder. Such bacteria include Bifidobacterium bacteria. Thus, in some embodiments, the composition is substantially free of Bacteroides bacteria. A composition is substantially free of a bacterial type if that type makes up less than 10% of the bacteria in a composition, preferably less than 5%, even more preferably less than 1%, most preferably less than 0.5%, or even 0% of the bacteria in the composition.

In some embodiments, the composition comprises a fecal sample comprising at least one bacteria that regulate a bile salt and/or a bile acid, such as any bacteria listed in Table 4. In some embodiments, the fecal sample is from a fecal bank. In some embodiments, the compositions may be added to a fecal sample prior to administration to the subject.

In some embodiments, provided herein are methods of treating or preventing a metabolic condition, by administering a composition (e.g., a fecal sample) that regulate a bile salt and/or a bile acid, such as any bacteria listed in Table 4. The fecal sample is enriched if at least 0.01%, at least 0.02%, at least 0.03%, at least 0.04%, at least 0.05%, at least 0.06%, at least 0.07%, at least 0.08%, at least .09%, at least 0.1%, at least 0.2%, at least 0.3%, at least 0.4%, at least 0.5%, at least 0.6%, at least 0.7%, at least 0.8%, at least 0.9%, at least 1%, or at least 2%, at least 3%, at least 4%, at least 5%, at least 6%, at least 7%, at least 8%, at least 9%, at least 10%, at least 15%, at least 20%, at least 25%, at least 30%, at least 35%, at least 40%, at least 45%, or at least 50% of the bacteria in the fecal sample is bacteria that regulate a bile salt and/or a bile acid, such as any bacteria listed in Table 4. In some embodiments, the fecal sample is from a fecal bank. In some embodiments, the fecal sample is from a donor.

The composition may further comprise a nutrient. In some embodiments, the nutrient aids in the growth of bacteria (e.g., bacteria disclosed herein). In some embodiments, the nutrient is a lipid (e.g., lineoleic acid, stearic acid, or palmitic acid). In some embodiments, the nutrient may be conjointly administered with a composition disclosed herein. As used herein, the phrase “conjoint administration” refers to any form of administration of two or more different agents (e.g., a composition disclosed herein and a nutrient disclosed herein) such that the second agent is administered while the previously administered agent is still effective in the body. For example, the compositions disclosed herein and the nutrients disclosed herein can be administered either in the same formulation or in a separate formulation, either concomitantly or sequentially.

Actual dosage levels of the active ingredients in the pharmaceutical compositions may be varied so as to obtain an amount of the active ingredient which is effective to achieve the desired therapeutic response for a particular patient, composition, and mode of administration, without being toxic to the patient.

The selected dosage level will depend upon a variety of factors including the activity of the particular agent employed, the route of administration, the time of administration, the rate of excretion or metabolism of the particular compound being employed, the duration of the treatment, other drugs, compounds and/or materials used in combination with the particular compound employed, the age, sex, weight, condition, general health and prior medical history of the patient being treated, and like factors well known in the medical arts.

A physician or veterinarian having ordinary skill in the art can readily determine and prescribe the effective amount of the pharmaceutical composition required. For example, the physician or veterinarian could prescribe and/or administer doses of the compounds employed in the pharmaceutical composition at levels lower than that required in order to achieve the desired therapeutic effect and gradually increase the dosage until the desired effect is achieved.

### EXEMPLIFICATION

### Example 1: Genomic Comparison Reveal Distinct Sub Groups of Turicibacter sanguinis

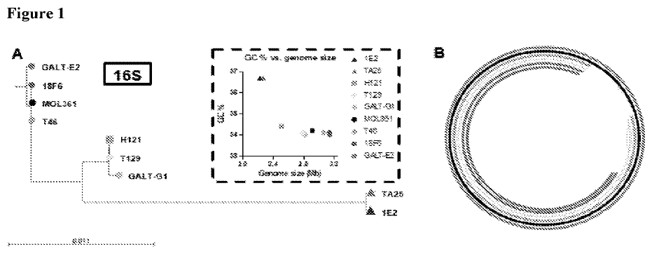

FIG. 1A & FIG. 1B show genomic comparison reveal distinct sub groups of Turicibacter sanguinis. A: Phylogenetic tree comparing full-length 16S rRNA sequences from noted T. sanguinis isolates. Circles indicate human-derived isolated, triangles indicate mouse-derived isolates, and squares indicate mouse-derived contaminating isolate. B: Full shotgun-assembled genome comparisons of T. sanguinis isolates listed in A.

### Example 2: T. Sanguinis Isolates Differ in their Bile-Modifying Abilities

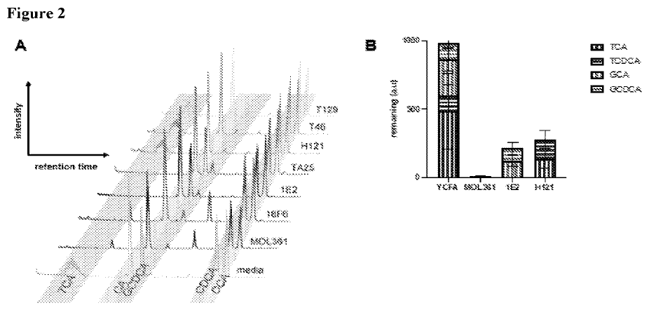

FIG. 2A & FIG. 2B show T. sanguinis isolates differ in their bile-modifying abilities. A: Liquid chromatograms of individual T. sanguinis isolates grown for 24 hours in media with sub-inhibitory concentrations of five bile salts/acids: taurocholic acid (TCA), cholic acid (CA), glycochenodeoxycholic acid (GCDCA), chenodeoxycholic acid (CDCA), and deoxycholic acid (DCA). Shaded regions indicate expected retention time of each bile species. B: Relative amounts of remaining conjugated bile salts (tauricholic acid, taurochenodeoxycholic acid, glycocholic acid, glycochenodeoxycholic acid) after 24 growth with noted T. sanguinis isolate. Magenta=glycine-conjugated bile salts, blue=taurine-conjugated bile salts.

### Example 3: T. Sanguinis Isolates Differ in their Genetic Capacity to Modify Bile Species

Table 2 shows presence (+) or absence (−) of sequence homologs with potential bile modifying activity in T. sanguinis isolates. For 7-alpha hydroxysteroid dehydrogenase homologs (far right column), percent amino acid identify with Clostridium absonum is also shown.

TABLE 2

| | BSH | BSH | BSH | BSH | BSH | BSH | BSH | BSH | |
|---|---|---|---|---|---|---|---|---|---|
| | Group | Group | Group | Group | Group | Group | Group | Group | 7α- |
| Isolate | I | II | III | IV | V | VI | VII | VIII | HSDH |
| MOL361 | + | − | − | + | + | − | − | + | 57% |
| 18F6 | + | − | − | − | + | − | − | + | 57% |
| T46 | + | − | − | − | + | − | − | + | 57% |
| GALT- | + | − | − | − | + | − | − | + | ? |
| E2 | | | | | | | | | |
| H121 | − | + | − | − | − | + | − | − | 64% |
| T129 | − | + | − | − | − | + | − | − | 64% |
| GALT- | − | + | − | − | − | + | − | − | ? |
| G1 | | | | | | | | | |
| 1E2 | − | − | + | + | − | − | + | − | − |
| TA25 | − | − | + | + | − | − | + | − | − |

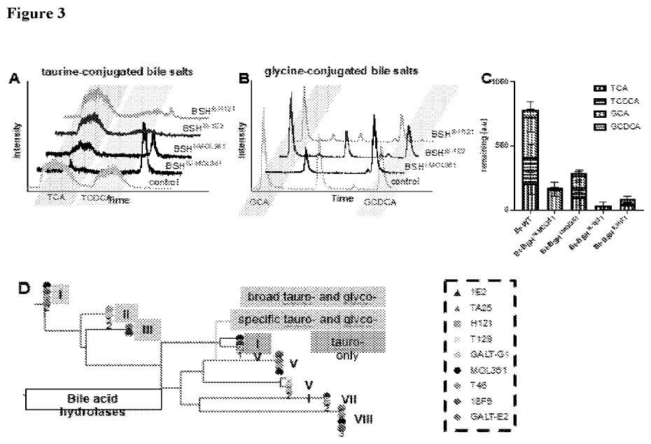

FIG. 3A-FIG. 3D show T. sanguinis isolates differ in their genetic capacity to modify bile species. A: Liquid chromatograms of media after 24 hour cultures of E. coli expressing individual predicted bile salt hydrolases (BSH) from each sequence grouping and isolate and grown in with TCA and TCDCA. Control is E. coli with same expression vector but expressing non-bile modifying gene. B: same as A, but with GCA and GCDCA instead of taurine-conjugated bile salts. C. Remaining amounts of TCA, TCDCA, GCA, and GCDCA after 24 hour growths of E. coli expressing noted BSH homologs. D: Phylogenetic tree of each predicted BSH gene from T. sanguinis isolates, with bile salt specificity noted in boxes. We did not detect bile salt hydrolase activity in sequences without boxes.

### Example 4: T. Sanguinis BSH Expression is Sufficient to Alter Host Lipidome and Health-Associated Lipid Markers

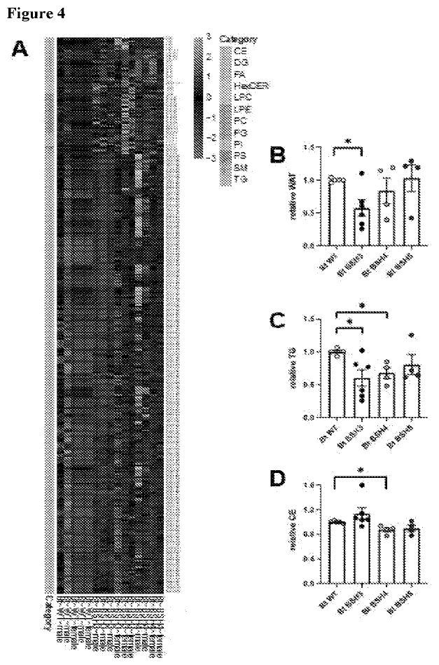

FIGS. 4A-FIG. 4D show T. sanguinis BSH expression is sufficient to alter host lipidome and health-associated lipid markers. A: Heatmap of plasma lipid species significantly altered by expression of at least one T. sanguinis BSH in Bacteroides thetaiotaomicron. Colors on left correspond to lipid class, cyan-magenta colorscale represents Z-score. B. Relative white adipose tissue weight of mice monocolonized with BSH-expression B. thetatiotaomicron. C. Relative combined plasma triglycerides (TG) of mice monocolonized with BSH-expression B. thetatiotaomicron. D. Relative cholesterol esters (CE) of mice monocolonized with BSH-expression B. thetatiotaomicron. All values normalized to sex-matched littermates, *=p<0.05 two-tailed t-test.

### Example 5: T. Sanguinis Isolates Differ in their Effects on Host Lipid Biology and Bile Acids

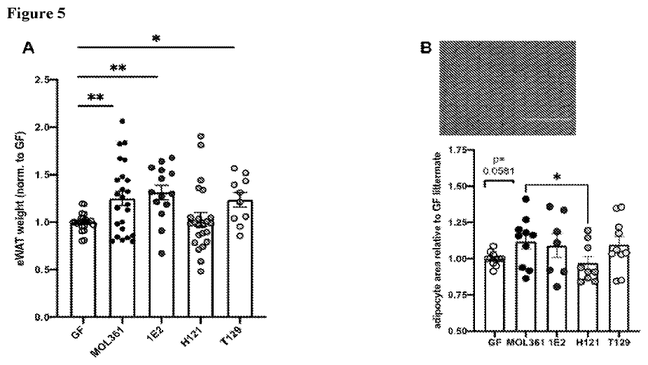

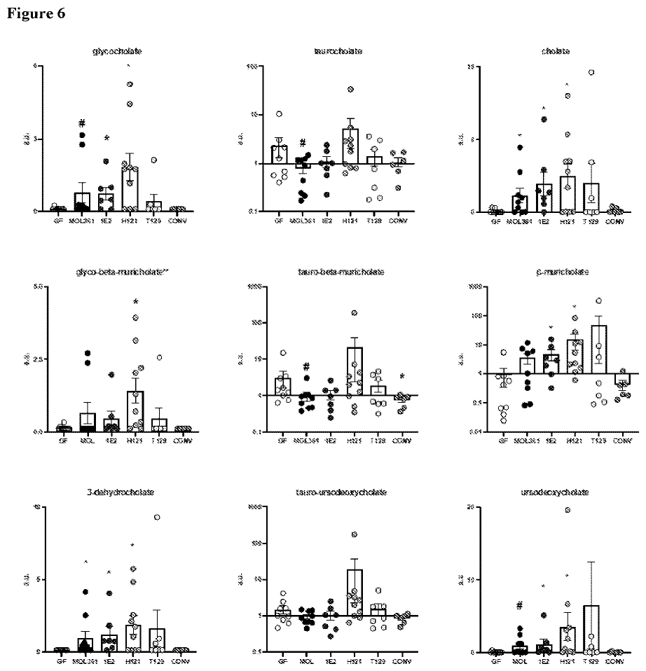

FIG. 5A-FIG. 5B show T. sanguinis isolates differ in their effects on host lipid biology and bile acids. A: Sex and littermatched relative abdominal fat pad mass from mice monocolonized with individual T. sanguinis isolates. B: top: Example image of adipose tissue histology section. bottom: Sex and litter matched relative adipocyte size of mice monocolonized with individual T. sanguinis isolates.

### Example 6: T. Sanguinis Isolates Differ in their Effects on Host Lipid Biology and Bile Acids

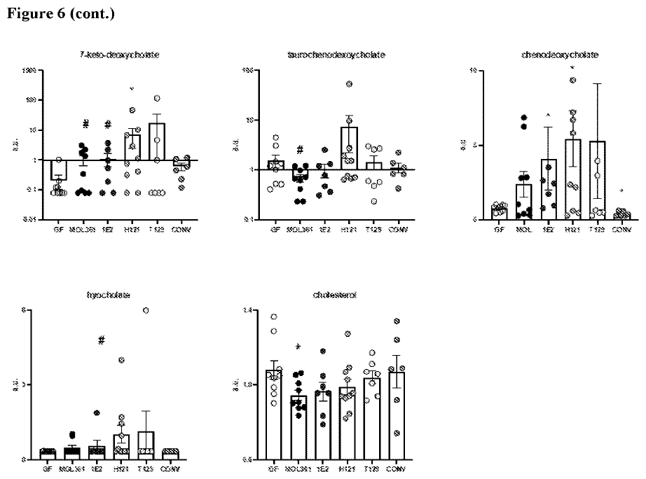

FIG. 6 shows levels of circulating serum bile acids and cholesterol in mice monocolonzed by individual T. sanguinis isolates. #=p<0.1, *=p<0.05.

### Example 7: Turicibacter Modifies Host Bile Acids and Lipids in a Strain-Specific Manner

Bacteria from the Turicibacter genus are prominent members of the mammalian gut microbiota and are associated with alterations in dietary fat and body weight, but the specific connections between these symbionts and host physiology are poorly understood. A a diverse set of mouse- and human-derived Turicibacter strains were genomically and phenotypically characterized, and found they group into three clades that differ in their transformations of bile acids. Turicibacter bile salt hydrolases that confer strain-specific differences in bile deconjugation were identified. Colonization with individual Turicibacter strains led to changes in host bile acid profiles, generally aligning with those produced in vitro. Further, colonizing mice with another bacterium expressing bile-modifying genes from these strains decreased serum cholesterol, and triglycerides, and as well as adipose tissue mass. This work identifies genes that enable diverse Turicibacter strains to differentially modify host bile acids and lipid metabolism, and positions multiple Turicibacter strains as candidates for altering host fat biology.

### INTRODUCTION

The gut microbiota forms complex relationships with its host organism, modulating broad aspects of host physiology including metabolism^{1,2} and neurobiology^{3,4}. Often, the connections between the gut microbiota and host physiology are easiest to decipher through presence/absence of large sectors of the microbial community (examples in ⁵⁻⁷), but in some cases, specific microbial features and/or taxa serve important roles in host physiology⁸⁻¹⁰.

The mammalian gut microbiota has long been associated with obesity^{25,26}, but studies often provide strong correlations rather than mechanistic determinants of these relationships, indicating a further need for fundamental interrogation into connections between the microbiota and host fat²⁷. Numerous microbiota community profiling studies reveal correlations between Turicibacter and features of host fat metabolism, such as adiposity and dictary lipids²⁸⁻³³, but the nature of these correlations varies^{34,35}. It was recently observed that the type strain of T. sanguinis, MOL361^{36,37}, broadly alters the host serum lipidome while decreasing serum cholesterol and triglycerides in mice³⁸. This same strain was also reported to modify bile species through deconjugation and dehydrogenation in vitro²¹, suggesting at least one potential means by which Turicibacter can influence host lipid status. Based on these findings, it is hypothesized that there may be variations in the functional activity of Turicibacter strains that account for differences in host bile and lipid biology, providing a mechanism to connect this taxon to aspects of host physiology.

### Results

Turicibacter Isolates Separate into Genetically Distinguishable Strains

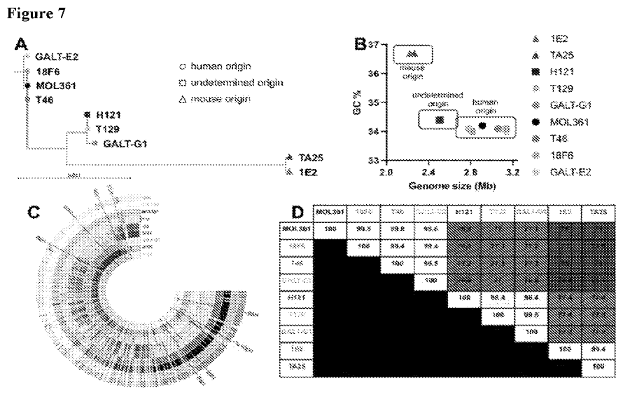

To better understand the diversity within the Turicibacter genus, nine isolates were gathered from the fecal microbiotas of mice and humans that had been identified as T. sanguinis based on their 16S rRNA gene sequence (97% full length 16S rRNA gene sequence similarity cutoff, Table 5). Two of these isolates had been previously identified (human-derived type strain MOL361 and H121, which was derived from contaminated germ-free mice^{36,39}); five had been isolated but not published (human isolates 18F6, T46, and T129, and mouse isolates 1E2 and TA25); and two were isolated from a human fecal sample specifically for this study (GALT-E2 and GALT-G1) using an array-based isolation and cultivation platform (see Methods). Shotgun short read sequencing was performed and draft assemblies of each isolate genome were created. Comparisons of the 16S rRNA gene phylogeny (FIG. 7a), general genome characteristics (FIG. 7b), or specific genome sequence (FIG. 7c, d) revealed that even with this fairly small sample of the 16S rRNA gene-based “T. sanguinis” species designation, there were at least three distinct subgroups: two from humans (exemplar isolates MOL361 and H121, with 99.3% full length 16S rRNA gene similarity), and one from mice (exemplar strain 1E2, 97.5% and 97.8% 16S rRNA gene similarity with MOL361 and H121, respectively). Genomic alignments indicated a substantial amount of shared DNA sequences within members of the same subgroups (all within group average nucleotide identity [ANI]>98.3%) with the remaining amount of genetic variation indicating smaller genetic differences between related isolates. These within-subgroup shared 5 sequences were distinct from members of the other two subgroups (intergroup ANI: MOL361-H121=76.80%, MOL361-1E2=74.95%, H121-1E2-77.43%). It is important to note that the H121-group genomically resembles the newly described species Turicibacter bilis¹³ (98.8% ANI), currently the only other named species from this genus. Overall, these genomic differences suggest distinct evolutionary histories that correspond at least partially with host origin.

TABLE 5

| | Derived | | | |
|---|---|---|---|---|
| Isolate | from | Source | Reference | Notes |
| MOL361 | human | DMSZ | Bosshard, | Turicibacter |
| | | | 2002 | sanguinis type strain |
| 18F6 | human | Kenya Honda | N/A | |
| T46 | human | Thomas | N/A | |
| | | Auchtung | | |
| GALT-G1 | human | this work | N/A | |
| H121 | contaminated | Thomas | Auchtung, | |
| | germ- | Auchtung | 2016 | |
| | free | | | |
| | mouse | | | |
| T129 | human | Thomas | N/A | |
| | | Auchtung | | |
| GALT-E2 | human | this work | N/A | |
| 1E2 (also known as 80E2) | mouse | Kenya Honda | N/A | |
| TA25 | mouse | Thomas | N/A | |
| | | Auchtung | | |
| Bacteroides | human | Justin | Xu, 2003 | background strain |
| thetaiotaomicron | | Sonnenburg/Fatima | | |
| VPI-5482 | | Enam | | |
| B. thetaiotaomicron | N/A | rederived for | Whitaker, | referred to as “WT” |
| pWW3837 | | this work | 2017 | in this work |
| Bt-BSH-IV: MOL361 | N/A | this work | N/A | WT B. |
| | | | | thetaiotaomicron |
| | | | | with BSH-IV from |
| | | | | MOL361 in place of |
| | | | | GFP |
| Bt-BSH-I: MOL361 | N/A | this work | N/A | WT B. |
| | | | | thetaiotaomicron |
| | | | | with BSH-I from |
| | | | | MOL361 in place of |
| | | | | GFP |
| Bt-BSH-II: H121 | N/A | this work | N/A | WT B. |
| | | | | thetaiotaomicron |
| | | | | with BSH-II from |
| | | | | H121 in place of |
| | | | | GFP |
| Bt-BSH-III: 1E2 | N/A | this work | N/A | WT B. |
| | | | | thetaiotaomicron |
| | | | | with BSH-III from |
| | | | | 1E2 in place of GFP |
| Escherichia coli C41-pLys | N/A | Lucigen | N/A | expression strain |
| Escherichia coli WM3064 | N/A | Brittany | Lynch, | conjugation donor |
| | | Bennett | 2019 | |

Turicibacter Isolates Differ in their Impact on Host Fat Biology and Circulating Metabolome

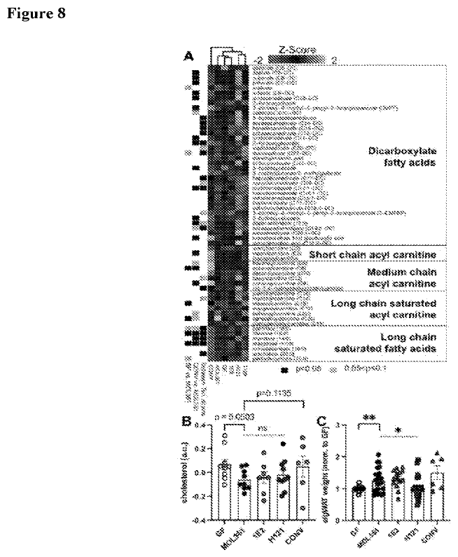

Previous findings revealed that monocolonizing mice with T. sanguinis MOL361 altered host fat tissue and circulating lipids³⁸. Due to the large genomic variation between our Turicibacter strains, it was predicted that they would vary in their effects on host lipid biology. Representative isolates from each of the distinct phylogenetic subgroups (MOL361, H121 and T129, and 1E2) were chosedn and measured their effects on circulating metabolites and adipose tissue in monocolonized mice relative to germ-free (GF) and conventionalized controls (i.e. gavaged with complete microbiota, CONV). Compared to GF littermates, CONV mice had decreased levels of several dicarboxylate fatty acids, long-chain fatty acids, and long-chain acyl carnitines, with a broad increase in short- and medium-chain acyl carnitines (FIG. 8a). Consistent with a previous report³⁸, colonization with individual Turicibacter strains also induced widespread alterations in host serum lipids, with many strain-level differences in host lipid alterations (FIG. 8a, Table 6). Compared to GF controls, MOL361 increased a subset of long-chain acyl carnitines, and decreased many long-chain saturated fatty acids and dicarboxylic acids. In addition, colonization with MOL361 elicited significant decreases in host cholesterol to levels below those seen in both GF and CONV animals (FIG. 8b). Compared to GF, H121 colonization significantly increased serum levels of several medium-chain fatty acids, dicarboxylic acids, and short-, medium- and long-chain acyl carnitines. 1E2 colonization had a smaller overall effect on host lipids, but led to a decrease in several dicarboxlyate fatty acids (FIG. 8a), In addition to differences between GF or CONV mice and Turicibacter-monocolonized animals, there were also broad alterations in host lipids in response to colonization with different Turicibacter strains, with sizeable discrepancies in dicarboxylate and long chain saturated fatty acids (FIG. 8a) At the tissue level, two of the four Turicibacter strains stimulated statistically significant increases in epididymal/gonadal white adipose tissue (e/gWAT) mass in comparison to GF controls, and a third strain elicited similar increases that were not statistically significant. In contrast, there was no noticeable effect of H121 (FIG. 8c). Consistent with this, H121 showed the smallest e/gWAT adipocyte size within fat pads (FIG. 13a-f). This may be due to lower colonization of H121 in both the small intestine and the colon (FIG. 13g, h) These results indicate that there is not necessarily a connection between changes in specific lipid species and mass of adipose tissue.

TABLE 6

| | | Fold Change | | | | |
|---|---|---|---|---|---|---|
| | | Welch′s Two-Sample t-Test | | | | |
| | | (bold indicates p < 0.1) | | | | |
| | | MOL361 | 1E2 | H121 | T129 | CON |
| Sub Pathway | Biochemical Name | GF | GF | GF | GF | GF |
| Fatty Acid, Branched | (14 or 15)-methylpalmitate (a17:0 or i17:0) | 0.66 | 1.07 | 1.14 | 1.06 | 0.57 |
| Fatty Acid, Branched | (16 or 17)-methylstearate (a19:0 or i19:0) | 0.66 | 1.13 | 1.01 | 1.08 | 0.36 |
| Medium Chain Fatty Acid | (2 or 3)-decenoate (10:1n7 or n8) | 0.82 | 0.93 | 1.78 | 1.05 | 0.39 |
| Polyamine Metabolism | (N(1) + N(8))-acetylspermidine | 1.00 | 1.10 | 0.79 | 1.18 | 1.04 |
| Fatty Acid Metabolism (Acyl Carnitine, | (R)-3-hydroxybutyrylcarnitine | 1.55 | 1.76 | 1.93 | 1.47 | 2.63 |
| Hydroxy) | | | | | | |
| Fatty Acid Metabolism (Acyl Carnitine, | (S)-3-hydroxybutyrylcarnitine | 1.27 | 1.32 | 1.44 | 1.11 | 1.56 |
| Hydroxy) | | | | | | |
| Lysoplasmalogen | 1-(1-enyl-oleoyl)-GPE (P-18:1)* | 1.08 | 1.17 | 1.58 | 1.02 | 0.92 |
| Plasmalogen | 1-(1-enyl-palmitoyl)-2-arachidonoyl-GPC (P- | 0.94 | 1.08 | 1.20 | 1.02 | 1.36 |
| | 16:0/20:4)* | | | | | |
| Plasmalogen | 1-(1-enyl-palmitoyl)-2-arachidonoyl-GPE (P- | 1.06 | 1.39 | 1.26 | 1.16 | 1.17 |
| | 16:0/20:4)* | | | | | |
| Plasmalogen | 1-(1-enyl-palmitoyl)-2-linoleoyl-GPC (P- | 0.82 | 0.98 | 0.95 | 0.99 | 1.38 |
| | 16:0/18:2)* | | | | | |
| Plasmalogen | 1-(1-enyl-palmitoyl)-2-linoleoyl-GPE (P- | 1.04 | 1.60 | 1.26 | 1.38 | 1.41 |
| | 16:0/18:2)* | | | | | |
| Plasmalogen | 1-(1-enyl-palmitoyl)-2-oleoyl-GPC (P- | 1.02 | 1.22 | 1.33 | 1.18 | 1.14 |
| | 16:0/18:1)* | | | | | |
| Plasmalogen | 1-(1-enyl-palmitoyl)-2-oleoyl-GPE (P- | 1.21 | 1.37 | 1.20 | 1.39 | 1.42 |
| | 16:0/18:1)* | | | | | |
| Plasmalogen | 1-(1-enyl-palmitoyl)-2-palmitoyl-GPC (P- | 0.90 | 0.96 | 1.12 | 1.02 | 0.99 |
| | 16:0/16:0)* | | | | | |
| Lysoplasmalogen | 1-(1-enyl-palmitoyl)-GPC (P-16:0)* | 0.96 | 1.02 | 0.96 | 0.98 | 0.93 |
| Lysoplasmalogen | 1-(1-enyl-palmitoyl)-GPE (P-16:0)* | 0.94 | 1.09 | 1.20 | 0.96 | 0.87 |
| Plasmalogen | 1-(1-enyl-stearoyl)-2-arachidonoyl-GPE (P- | 1.04 | 1.19 | 1.24 | 0.92 | 1.20 |
| | 18:0/20:4)* | | | | | |
| Plasmalogen | 1-(1-enyl-stearoyl)-2-linoleoyl-GPE (P- | 1.03 | 1.25 | 1.17 | 1.00 | 1.42 |
| | 18:0/18:2)* | | | | | |
| Plasmalogen | 1-(1-enyl-stearoyl)-2-oleoyl-GPE (P-18:0/18:1) | 1.22 | 1.40 | 1.29 | 1.22 | 1.95 |
| Lysoplasmalogen | 1-(1-enyl-stearoyl)-GPE (P-18:0)* | 1.05 | 1.10 | 1.10 | 0.97 | 1.00 |
| Lysophospholipid | 1-arachidonoyl-GPA (20:4) | 1.08 | 1.23 | 1.13 | 1.12 | 0.63 |
| Lysophospholipid | 1-arachidonoyl-GPC (20:4n6)* | 0.93 | 0.97 | 0.98 | 1.06 | 0.69 |
| Lysophospholipid | 1-arachidonoyl-GPE (20:4n6)* | 1.01 | 0.97 | 0.94 | 0.98 | 0.53 |
| Lysophospholipid | 1-arachidonoyl-GPI (20:4)* | 0.61 | 0.84 | 0.62 | 0.63 | 0.43 |
| Monoacylglycerol | 1-arachidonylglycerol (20:4) | 0.84 | 1.25 | 0.93 | 1.21 | 0.71 |
| Monoacylglycerol | 1-dihomo-linolenylglycerol (20:3) | 0.89 | 1.32 | 0.96 | 1.14 | 0.54 |
| Monoacylglycerol | 1-docosahexaenoylglycerol (22:6) | 0.95 | 1.30 | 0.84 | 1.14 | 0.64 |
| Monoacylglycerol | 1-eicosapentaenoylglycerol (20:5)* | 0.71 | 1.09 | 0.80 | 1.09 | 0.70 |
| Monoacylglycerol | 1-heptadecenoylglycerol (17:1)* | 0.78 | 1.23 | 0.98 | 1.03 | 0.59 |
| Lysophospholipid | 1-lignoceroyl-GPC (24:0) | 0.89 | 0.93 | 0.86 | 1.04 | 0.78 |
| Lysophospholipid | 1-linolenoyl-GPC (18:3)* | 0.71 | 0.85 | 0.89 | 0.89 | 0.79 |
| Monoacylglycerol | 1-linolenoylglycerol (18:3) | 0.71 | 1.14 | 0.72 | 1.07 | 0.57 |
| Phosphatidylcholine (PC) | 1-linoleoyl-2-arachidonoyl-GPC (18:2/20:4n6)* | 0.90 | 1.05 | 0.98 | 1.01 | 0.85 |
| Phosphatidylcholine (PC) | 1-linoleoyl-2-linolenoyl-GPC (18:2/18:3)* | 0.85 | 0.99 | 0.94 | 1.16 | 1.24 |
| Lysophospholipid | 1-linoleoyl-GPA (18:2)* | 0.78 | 1.10 | 0.97 | 0.91 | 0.65 |
| Lysophospholipid | 1-linoleoyl-GPC (18:2) | 0.84 | 0.98 | 0.97 | 1.04 | 0.83 |
| Lysophospholipid | 1-linoleoyl-GPE (18:2)* | 0.79 | 0.90 | 0.85 | 1.02 | 0.58 |
| Lysophospholipid | 1-linoleoyl-GPG (18:2)* | 0.63 | 0.98 | 0.85 | 1.05 | 0.54 |
| Lysophospholipid | 1-linoleoyl-GPI (18:2)* | 0.52 | 0.81 | 0.60 | 0.63 | 0.40 |
| Lysophospholipid | 1-linoleoyl-GPS (18:2)* | 1.13 | 1.40 | 1.02 | 1.30 | 0.41 |
| Monoacylglycerol | 1-linoleoylglycerol (18:2) | 0.79 | 1.16 | 0.84 | 1.02 | 0.48 |
| Histidine Metabolism | 1-methyl-4-imidazoleacetate | 1.19 | 1.05 | 1.20 | 0.93 | 0.95 |
| Histidine Metabolism | 1-methyl-5-imidazoleacetate | 0.96 | 1.41 | 1.11 | 1.14 | 1.04 |
| Histidine Metabolism | 1-methyl-5-imidazolelactate | 0.96 | 1.28 | 1.08 | 1.05 | 1.01 |
| Guanidino and Acetamido Metabolism | 1-methylguanidine | 0.85 | 0.89 | 0.85 | 0.97 | 1.32 |
| Histidine Metabolism | 1-methylhistamine | 1.50 | 1.27 | 1.16 | 0.87 | 0.66 |
| Histidine Metabolism | 1-methylhistidine | 1.22 | 1.46 | 1.32 | 1.13 | 1.28 |
| Nicotinate and Nicotinamide Metabolism | 1-methylnicotinamide | 0.87 | 0.95 | 0.91 | 0.75 | 0.79 |
| Phosphatidylcholine (PC) | 1-myristoyl-2-arachidonoyl-GPC (14:0/20:4)* | 0.97 | 1.01 | 0.99 | 1.01 | 1.13 |
| Phosphatidylcholine (PC) | 1-myristoyl-2-palmitoyl-GPC (14:0/16:0) | 0.93 | 1.03 | 1.04 | 1.15 | 1.09 |
| Monoacylglycerol | 1-myristoylglycerol (14:0) | 0.65 | 1.05 | 0.69 | 0.96 | 0.43 |
| Phosphatidylethanolamine (PE) | 1-oleoyl-2-arachidonoyl-GPE (18:1/20:4)* | 1.23 | 1.16 | 1.05 | 1.23 | 1.14 |
| Phosphatidylinositol (PI) | 1-oleoyl-2-arachidonoyl-GPI (18:1/20:4)* | 0.89 | 1.14 | 0.99 | 1.01 | 0.82 |
| Phosphatidylcholine (PC) | 1-oleoyl-2-docosahexaenoyl-GPC (18:1/22:6)* | 1.18 | 1.48 | 1.21 | 1.22 | 0.90 |
| Phosphatidylethanolamine (PE) | 1-oleoyl-2-docosahexaenoyl-GPE (18:1/22:6)* | 1.17 | 0.98 | 1.16 | 1.07 | 1.44 |
| Phosphatidylcholine (PC) | 1-oleoyl-2-linoleoyl-GPC (18:1/18:2)* | 0.87 | 1.10 | 1.05 | 1.01 | 0.70 |
| Phosphatidylethanolamine (PE) | 1-oleoyl-2-linoleoyl-GPE (18:1/18:2)* | 0.95 | 1.13 | 1.03 | 1.13 | 1.15 |
| Lysophospholipid | 1-oleoyl-GPC (18:1) | 0.92 | 1.08 | 0.99 | 1.04 | 0.90 |
| Lysophospholipid | 1-oleoyl-GPE (18:1) | 0.91 | 1.12 | 1.04 | 1.04 | 0.79 |
| Lysophospholipid | 1-oleoyl-GPI (18:1) | 0.51 | 0.93 | 0.48 | 0.45 | 0.36 |
| Monoacylglycerol | 1-oleoylglycerol (18:1) | 1.12 | 1.74 | 0.74 | 1.59 | 0.54 |
| Phosphatidylcholine (PC) | 1-palmitoleoyl-2-linolenoyl-GPC (16:1/18:3)* | 0.84 | 0.96 | 1.01 | 1.08 | 1.33 |
| Phosphatidylcholine (PC) | 1-palmitoleoyl-2-linoleoyl-GPC (16:1/18:2)* | 0.99 | 1.18 | 1.09 | 1.15 | 1.22 |
| Lysophospholipid | 1-palmitoleoyl-GPC (16:1)* | 0.87 | 1.13 | 1.03 | 0.99 | 1.02 |
| Monoacylglycerol | 1-palmitoleoylglycerol (16:1)* | 0.65 | 1.04 | 0.68 | 0.87 | 0.52 |
| Phosphatidylcholine (PC) | 1-palmitoyl-2-arachidonoyl-GPC (16:0/20:4n6) | 1.02 | 1.06 | 1.04 | 1.13 | 0.98 |
| Phosphatidylethanolamine (PE) | 1-palmitoyl-2-arachidonoyl-GPE (16:0/20:4)* | 1.00 | 0.94 | 0.85 | 1.03 | 1.08 |
| Phosphatidylinositol (PI) | 1-palmitoyl-2-arachidonoyl-GPI (16:0/20:4)* | 0.79 | 0.79 | 0.80 | 0.94 | 0.90 |
| Phosphatidylcholine (PC) | 1-palmitoyl-2-dihomo-linolenoyl-GPC | 0.99 | 1.18 | 1.10 | 1.17 | 0.93 |
| | (16:0/20:3n3 or 6)* | | | | | |
| Phosphatidylcholine (PC) | 1-palmitoyl-2-docosahexaenoyl-GPC | 1.04 | 1.04 | 1.01 | 1.15 | 1.09 |
| | (16:0/22:6) | | | | | |
| Phosphatidylethanolamine (PE) | 1-palmitoyl-2-docosahexaenoyl-GPE | 0.94 | 0.74 | 0.83 | 0.85 | 1.44 |
| | (16:0/22:6)* | | | | | |
| Phosphatidylcholine (PC) | 1-palmitoyl-2-gamma-linolenoyl-GPC | 0.74 | 0.62 | 0.82 | 0.91 | 0.85 |
| | (16:0/18:3n6)* | | | | | |
| Phosphatidylcholine (PC) | 1-palmitoyl-2-linoleoyl-GPC (16:0/18:2) | 0.96 | 1.08 | 1.04 | 1.18 | 1.01 |
| Phosphatidylethanolamine (PE) | 1-palmitoyl-2-linoleoyl-GPE (16:0/18:2) | 0.84 | 0.95 | 0.84 | 1.09 | 1.18 |
| Phosphatidylinositol (PI) | 1-palmitoyl-2-linoleoyl-GPI (16:0/18:2) | 0.69 | 0.77 | 0.81 | 0.94 | 0.89 |
| Phosphatidylcholine (PC) | 1-palmitoyl-2-oleoyl-GPC (16:0/18:1) | 0.97 | 1.02 | 1.02 | 1.10 | 1.15 |
| Phosphatidylethanolamine (PE) | 1-palmitoyl-2-oleoyl-GPE (16:0/18:1) | 1.19 | 1.08 | 1.09 | 1.22 | 1.38 |
| Phosphatidylinositol (PI) | 1-palmitoyl-2-oleoyl-GPI (16:0/18:1)* | 0.80 | 0.88 | 0.89 | 0.99 | 0.99 |
| Phosphatidylcholine (PC) | 1-palmitoyl-2-palmitoleoyl-GPC (16:0/16:1)* | 0.82 | 0.95 | 0.99 | 1.01 | 1.22 |
| Phosphatidylcholine (PC) | 1-palmitoyl-2-stearoyl-GPC (16:0/18:0) | 1.00 | 0.87 | 0.98 | 1.12 | 1.07 |
| Lysophospholipid | 1-palmitoyl-GPA (16:0) | 0.84 | 1.06 | 0.88 | 0.83 | 0.84 |
| Lysophospholipid | 1-palmitoyl-GPC (16:0) | 0.94 | 0.99 | 0.94 | 1.01 | 0.95 |
| Lysophospholipid | 1-palmitoyl-GPE (16:0) | 0.91 | 0.91 | 0.85 | 0.98 | 1.07 |
| Lysophospholipid | 1-palmitoyl-GPG (16:0)* | 0.44 | 0.80 | 0.60 | 0.84 | 0.65 |
| Lysophospholipid | 1-palmitoyl-GPI (16:0) | 0.50 | 0.75 | 0.41 | 0.49 | 0.41 |
| Monoacylglycerol | 1-palmitoylglycerol (16:0) | 0.78 | 1.14 | 1.01 | 1.13 | 0.57 |
| Monoacylglycerol | 1-pentadecanoylglycerol (15:0) | 0.74 | 1.14 | 0.92 | 1.06 | 0.57 |
| Histidine Metabolism | 1-ribosyl-imidazoleacetate* | 0.90 | 1.42 | 0.74 | 0.96 | 1.02 |
| Phosphatidylcholine (PC) | 1-stearoyl-2-arachidonoyl-GPC (18:0/20:4) | 1.01 | 0.94 | 0.99 | 1.07 | 0.91 |
| Phosphatidylethanolamine (PE) | 1-stearoyl-2-arachidonoyl-GPE (18:0/20:4) | 1.00 | 0.84 | 0.85 | 0.97 | 1.05 |
| Phosphatidylinositol (PI) | 1-stearoyl-2-arachidonoyl-GPI (18:0/20:4) | 0.95 | 0.94 | 0.96 | 1.03 | 0.99 |
| Phosphatidylserine (PS) | 1-stearoyl-2-arachidonoyl-GPS (18:0/20:4) | 2.43 | 1.21 | 2.07 | 2.74 | 1.63 |
| Phosphatidylcholine (PC) | 1-stearoyl-2-docosahexaenoyl-GPC (18:0/22:6) | 1.04 | 0.88 | 0.94 | 1.11 | 1.15 |
| Phosphatidylethanolamine (PE) | 1-stearoyl-2-docosahexaenoyl-GPE (18:0/22:6)* | 0.88 | 0.66 | 0.85 | 0.90 | 1.44 |
| Phosphatidylcholine (PC) | 1-stearoyl-2-linoleoyl-GPC (18:0/18:2)* | 0.91 | 0.98 | 0.98 | 1.10 | 0.88 |
| Phosphatidylethanolamine (PE) | 1-stearoyl-2-linoleoyl-GPE (18:0/18:2)* | 0.82 | 0.81 | 0.79 | 0.97 | 0.98 |
| Phosphatidylinositol (PI) | 1-stearoyl-2-linoleoyl-GPI (18:0/18:2) | 0.75 | 0.81 | 0.83 | 0.90 | 0.89 |
| Phosphatidylcholine (PC) | 1-stearoyl-2-oleoyl-GPC (18:0/18:1) | 0.95 | 0.94 | 0.96 | 1.04 | 1.04 |
| Phosphatidylethanolamine (PE) | 1-stearoyl-2-oleoyl-GPE (18:0/18:1) | 0.86 | 0.75 | 0.83 | 0.87 | 1.27 |
| Phosphatidylinositol (PI) | 1-stearoyl-2-oleoyl-GPI (18:0/18:1)* | 0.83 | 0.90 | 0.84 | 1.00 | 1.00 |
| Lysophospholipid | 1-stearoyl-GPC (18:0) | 0.96 | 0.89 | 0.87 | 0.97 | 0.84 |
| Lysophospholipid | 1-stearoyl-GPE (18:0) | 0.93 | 0.88 | 0.85 | 0.92 | 0.99 |
| Lysophospholipid | 1-stearoyl-GPI (18:0) | 0.56 | 0.78 | 0.47 | 0.46 | 0.43 |
| Phosphatidylcholine (PC) | 1,2-dilinoleoyl-GPC (18:2/18:2) | 0.83 | 0.96 | 0.95 | 1.05 | 0.94 |
| Phosphatidylethanolamine (PE) | 1,2-dilinoleoyl-GPE (18:2/18:2)* | 0.75 | 0.86 | 0.94 | 1.13 | 1.08 |
| Phosphatidylcholine (PC) | 1,2-dioleoyl-GPC (18:1/18:1) | 1.00 | 1.19 | 1.02 | 1.04 | 0.88 |
| Phosphatidylcholine (PC) | 1,2-dipalmitoyl-GPC (16:0/16:0) | 1.10 | 1.14 | 1.16 | 1.33 | 1.11 |
| Phosphatidylethanolamine (PE) | 1,2-dipalmitoyl-GPE (16:0/16:0)* | 1.67 | 1.24 | 1.31 | 1.61 | 1.33 |
| Glycolysis, Gluconeogenesis, and Pyruvate | 1,5-anhydroglucitol (1,5-AG) | 0.98 | 1.01 | 1.03 | 1.18 | 0.59 |
| Metabolism | | | | | | |
| Long Chain Monounsaturated Fatty Acid | 10-heptadecenoate (17:1n7) | 0.82 | 1.27 | 1.28 | 1.21 | 0.55 |
| Long Chain Monounsaturated Fatty Acid | 10-nonadecenoate (19:1n9) | 0.78 | 1.31 | 1.20 | 1.18 | 0.44 |
| Medium Chain Fatty Acid | 10-undecenoate (11:1n1) | 0.74 | 0.61 | 1.17 | 0.73 | 0.90 |
| Eicosanoid | 12-HEPE | 1.19 | 1.14 | 1.61 | 1.65 | 0.45 |
| Eicosanoid | 12-HETE | 1.03 | 1.39 | 3.43 | 1.48 | 0.21 |
| Eicosanoid | 12-HHTrE | 0.53 | 1.91 | 6.01 | 1.07 | 0.18 |
| Fatty Acid, Dihydroxy | 12,13-DiHOME | 1.14 | 0.95 | 1.54 | 1.52 | 0.77 |
| Fatty Acid, Monohydroxy | 13-HODE + 9-HODE | 0.93 | 1.04 | 1.22 | 1.38 | 0.60 |
| Docosanoid | 14-HDoHE/17-HDoHE | 1.22 | 1.17 | 1.57 | 1.78 | 0.43 |
| Fatty Acid, Monohydroxy | 16-hydroxypalmitate | 0.91 | 0.91 | 1.26 | 1.01 | 0.74 |
| Fatty Acid, Branched | 18-methylnonadecanoate (i20:0) | 0.71 | 1.02 | 1.16 | 1.45 | 0.42 |
| Benzoate Metabolism | 2-(4-hydroxyphenyl)propionate | 1.00 | 1.00 | 1.00 | 1.00 | 1.66 |
| Benzoate Metabolism | 2-amino-p-cresol sulfate | 0.85 | 0.77 | 0.81 | 0.77 | 35.50 |
| Lysine Metabolism | 2-aminoadipate | 1.00 | 0.84 | 0.98 | 1.27 | 1.03 |
| Glutathione Metabolism | 2-aminobutyrate | 1.01 | 0.96 | 1.04 | 1.06 | 1.20 |
| Fatty Acid, Amino | 2-aminoheptanoate | 0.96 | 1.09 | 1.14 | 0.86 | 1.02 |
| Fatty Acid, Amino | 2-aminooctanoate | 1.21 | 1.31 | 1.53 | 1.17 | 0.75 |
| Food Component/Plant | 2-aminophenol sulfate | 0.78 | 1.36 | 0.77 | 0.67 | 16.52 |
| Monoacylglycerol | 2-arachidonoylglycerol (20:4) | 0.75 | 1.09 | 0.80 | 0.96 | 0.62 |
| Fatty Acid Metabolism (Acyl Glycine) | 2-butenoylglycine | 0.89 | 0.79 | 0.62 | 1.02 | 1.29 |
| Monoacylglycerol | 2-docosahexaenoylglycerol (22:6)* | 0.85 | 1.12 | 0.81 | 1.02 | 0.58 |
| Leucine, Isoleucine and Valine Metabolism | 2-hydroxy-3-methylvalerate | 0.31 | 0.27 | 0.31 | 0.47 | 0.28 |
| Methionine, Cysteine, SAM and Taurine | 2-hydroxy-4-(methylthio)butanoic acid | 0.68 | 0.62 | 0.89 | 0.90 | 4.06 |
| Metabolism | | | | | | |
| Fatty Acid, Dicarboxylate | 2-hydroxyadipate | 0.90 | 0.82 | 0.96 | 1.04 | 0.91 |
| Fatty Acid, Monohydroxy | 2-hydroxyarachidate* | 0.74 | 1.30 | 0.85 | 1.43 | 0.67 |
| Fatty Acid, Monohydroxy | 2-hydroxybehenate | 0.76 | 1.08 | 0.97 | 1.19 | 0.94 |
| Glutathione Metabolism | 2-hydroxybutyrate/2-hydroxyisobutyrate | 0.93 | 0.90 | 0.99 | 0.95 | 1.17 |
| Fatty Acid, Monohydroxy | 2-hydroxydecanoate | 0.89 | 0.83 | 1.43 | 1.06 | 0.88 |
| Fatty Acid, Dicarboxylate | 2-hydroxyglutarate | 0.89 | 1.01 | 1.27 | 1.28 | 1.28 |
| Benzoate Metabolism | 2-hydroxyhippurate (salicylurate) | 0.85 | 1.10 | 0.96 | 1.12 | 0.91 |
| Fatty Acid, Monohydroxy | 2-hydroxyoctanoate | 0.99 | 1.07 | 1.38 | 1.04 | 0.81 |
| Fatty Acid, Monohydroxy | 2-hydroxypalmitate | 0.66 | 1.03 | 0.80 | 0.90 | 0.56 |
| Phenylalanine Metabolism | 2-hydroxyphenylacetate | 0.84 | 0.58 | 1.00 | 0.86 | 0.94 |
| Fatty Acid, Dicarboxylate | 2-hydroxysebacate | 0.81 | 1.09 | 1.06 | 0.98 | 0.42 |
| Fatty Acid, Monohydroxy | 2-hydroxystearate | 0.64 | 1.07 | 0.71 | 0.85 | 0.63 |
| Food Component/Plant | 2-isopropylmalate | 0.55 | 0.46 | 0.71 | 0.51 | 1.62 |
| Food Component/Plant | 2-keto-3-deoxy-gluconate | 0.71 | 0.71 | 0.90 | 0.52 | 0.48 |
| Fructose, Mannose and Galactose Metabolism | 2-ketogulonate | 0.96 | 0.89 | 0.87 | 0.80 | 2.69 |
| Monoacylglycerol | 2-linoleoylglycerol (18:2) | 0.81 | 1.08 | 0.79 | 1.00 | 0.51 |
| Benzoate Metabolism | 2-methoxyhydroquinone sulfate (1) | 1.08 | 0.99 | 1.07 | 1.16 | 0.86 |
| Benzoate Metabolism | 2-methoxyhydroquinone sulfate (2) | 1.24 | 1.26 | 1.31 | 1.34 | 1.03 |
| Leucine, Isoleucine and Valine Metabolism | 2-methylbutyrylcarnitine (C5) | 1.26 | 1.15 | 1.21 | 1.02 | 1.47 |
| Leucine, Isoleucine and Valine Metabolism | 2-methylbutyrylglycine | 0.75 | 0.47 | 0.50 | 1.06 | 0.92 |
| TCA Cycle | 2-methylcitrate/homocitrate | 0.93 | 1.04 | 0.99 | 1.23 | 1.01 |
| Glycine, Serine and Threonine Metabolism | 2-methylserine | 1.45 | 1.62 | 2.21 | 1.84 | 0.99 |
| Ascorbate and Aldarate Metabolism | 2-O-methylascorbic acid | 1.01 | 0.86 | 0.84 | 0.89 | 1.23 |
| Monoacylglycerol | 2-oleoylglycerol (18:1) | 0.82 | 1.16 | 0.71 | 0.97 | 0.41 |
| Food Component/Plant | 2-oxindole-3-acetate | 0.89 | 1.09 | 0.97 | 0.96 | 1.19 |
| Lysine Metabolism | 2-oxoadipate | 1.01 | 1.26 | 1.47 | 1.20 | 1.16 |
| Urea cycle; Arginine and Proline Metabolism | 2-oxoarginine* | 0.77 | 0.93 | 0.84 | 1.16 | 0.97 |
| Lysophospholipid | 2-palmitoleoyl-GPC (16:1)* | 0.57 | 0.94 | 0.84 | 0.79 | 0.69 |
| Monoacylglycerol | 2-palmitoleoylglycerol (16:1)* | 0.64 | 0.93 | 0.65 | 0.81 | 0.52 |
| Lysophospholipid | 2-palmitoyl-GPC (16:0)* | 0.68 | 1.03 | 0.76 | 0.93 | 0.74 |
| Monoacylglycerol | 2-palmitoylglycerol (16:0) | 0.59 | 1.19 | 0.97 | 0.75 | 0.55 |
| Food Component/Plant | 2-piperidinone | 1.04 | 0.77 | 1.00 | 0.86 | 1.15 |
| Lysophospholipid | 2-stearoyl-GPE (18:0)* | 0.67 | 0.99 | 0.70 | 0.83 | 0.67 |
| Chemical | 2,2′-Methylenebis(6-tert-butyl-p-cresol) | 0.63 | 0.96 | 0.54 | 1.41 | 1.30 |
| Leucine, Isoleucine and Valine Metabolism | 2,3-dihydroxy-2-methylbutyrate | 1.04 | 1.23 | 1.18 | 1.50 | 1.28 |
| Methionine, Cysteine, SAM and Taurine | 2,3-dihydroxy-5-methylthio-4-pentenoate | 0.91 | 0.94 | 1.10 | 0.86 | 0.90 |
| Metabolism | (DMTPA)* | | | | | |
| Food Component/Plant | 2,3-dihydroxyisovalerate | 0.45 | 0.52 | 0.72 | 0.66 | 0.48 |
| Chemical | 2,4-di-tert-butylphenol | 0.71 | 1.14 | 0.83 | 1.09 | 0.80 |
| Drug - Topical Agents | 2,6-dihydroxybenzoic acid | 0.83 | 1.31 | 1.10 | 0.97 | 1.12 |
| Food Component/Plant | 2,8-quinolinediol sulfate | 1.00 | 1.00 | 1.00 | 1.00 | 2.27 |
| Pyrimidine Metabolism, Cytidine containing | 2′-deoxycytidine | 1.18 | 1.18 | 1.07 | 1.14 | 1.01 |
| Purine Metabolism, (Hypo)Xanthine/Inosine | 2′-deoxyinosine | 0.98 | 2.67 | 5.68 | 1.41 | 0.37 |
| containing | | | | | | |
| Pyrimidine Metabolism, Uracil containing | 2′-deoxyuridine | 1.14 | 0.80 | 0.88 | 1.05 | 1.44 |
| Pyrimidine Metabolism, Cytidine containing | 2′-O-methylcytidine | 0.95 | 0.79 | 0.90 | 0.93 | 0.84 |
| Pyrimidine Metabolism, Uracil containing | 2′-O-methyluridine | 0.87 | 0.75 | 0.81 | 0.95 | 0.94 |
| Fatty Acid, Dihydroxy | 2R,3R-dihydroxybutyrate | 0.90 | 1.03 | 0.98 | 1.10 | 0.84 |
| Fatty Acid, Dihydroxy | 2S,3R-dihydroxybutyrate | 0.98 | 0.99 | 0.93 | 1.05 | 1.13 |
| Benzoate Metabolism | 3-(2-hydroxyphenyl)propionate | 0.88 | 1.12 | 1.59 | 1.30 | 2.05 |
| Benzoate Metabolism | 3-(3-hydroxyphenyl)propionate sulfate | 1.00 | 1.00 | 1.00 | 1.00 | 3.63 |
| Tyrosine Metabolism | 3-(4-hydroxyphenyl)lactate | 0.97 | 0.95 | 0.88 | 0.91 | 0.93 |
| Benzoate Metabolism | 3-(4-hydroxyphenyl)propionate | 1.29 | 1.38 | 1.40 | 3.59 | 2.25 |
| Chemical | 3-acetylphenol sulfate | 0.74 | 0.74 | 0.97 | 0.74 | 6.01 |
| Urea cycle; Arginine and Proline Metabolism | 3-amino-2-piperidone | 1.02 | 1.13 | 1.13 | 1.14 | 0.94 |
| Pyrimidine Metabolism, Thymine containing | 3-aminoisobutyrate | 0.79 | 0.78 | 0.93 | 1.15 | 0.92 |
| Chemical | 3-bromo-5-chloro-2,6-dihydroxybenzoic acid* | 0.65 | 1.45 | 1.00 | 1.05 | 1.08 |
| Fatty Acid, Dicarboxylate | 3-carboxy-4-methyl-5-pentyl-2-furanpropionate | 0.76 | 1.08 | 0.94 | 0.91 | 0.66 |
| | (3-CMPFP)** | | | | | |
| Fatty Acid, Dicarboxylate | 3-carboxy-4-methyl-5-propyl-2-furanpropanoate | 0.80 | 0.86 | 0.92 | 0.98 | 1.21 |
| | (CMPF) | | | | | |
| Fatty Acid Metabolism (Acyl Carnitine, | 3-decenoylcarnitine | 1.11 | 1.05 | 1.60 | 1.39 | 0.72 |
| Monounsaturated) | | | | | | |
| Secondary Bile Acid Metabolism | 3-dehydrocholate | 6.49 | 8.20 | 12.43 | 10.8 | 1.00 |
| | | | | | 8 | |
| Food Component/Plant | 3-formylindole | 0.77 | 1.20 | 1.30 | 0.99 | 0.89 |
| Leucine, Isoleucine and Valine Metabolism | 3-hydroxy-2-ethylpropionate | 0.96 | 0.94 | 1.28 | 1.08 | 1.26 |
| Chemical | 3-hydroxy-2-methylpyridine sulfate | 0.95 | 0.76 | 0.87 | 0.79 | 1.32 |
| Mevalonate Metabolism | 3-hydroxy-3-methylglutarate | 1.08 | 0.93 | 0.90 | 1.05 | 1.11 |
| Fatty Acid, Dicarboxylate | 3-hydroxyadipate | 0.83 | 0.71 | 0.85 | 0.82 | 0.66 |
| Ketone Bodies | 3-hydroxybutyrate (BHBA) | 0.80 | 1.03 | 1.13 | 0.86 | 1.91 |
| Fatty Acid Metabolism (Acyl Glycine) | 3-hydroxybutyroylglycine** | 1.42 | 1.46 | 1.23 | 3.02 | 2.83 |
| Fatty Acid, Monohydroxy | 3-hydroxydecanoate | 1.05 | 1.07 | 1.88 | 1.20 | 0.49 |
| Fatty Acid Metabolism (Acyl Carnitine, | 3-hydroxydecanoylcarnitine | 1.43 | 1.31 | 1.78 | 1.59 | 1.00 |
| Hydroxy) | | | | | | |
| Fatty Acid, Dicarboxylate | 3-hydroxydodecanedioate* | 0.94 | 0.68 | 1.69 | 1.07 | 0.68 |
| Fatty Acid, Monohydroxy | 3-hydroxyhexanoate | 1.08 | 0.83 | 1.90 | 0.91 | 2.74 |
| Fatty Acid Metabolism (Acyl Carnitine, | 3-hydroxyhexanoylcarnitine (1) | 1.30 | 1.34 | 1.74 | 1.43 | 1.39 |
| Hydroxy) | | | | | | |
| Fatty Acid Metabolism (Acyl Carnitine, | 3-hydroxyhexanoylcarnitine (2) | 1.94 | 1.50 | 2.32 | 1.27 | 1.80 |
| Hydroxy) | | | | | | |
| Leucine, Isoleucine and Valine Metabolism | 3-hydroxyisobutyrate | 0.65 | 0.78 | 1.00 | 0.94 | 0.88 |
| Fatty Acid, Monohydroxy | 3-hydroxylaurate | 1.04 | 0.97 | 1.87 | 1.16 | 0.36 |
| Fatty Acid, Monohydroxy | 3-hydroxymyristate | 0.91 | 0.83 | 1.46 | 1.04 | 0.38 |
| Fatty Acid, Monohydroxy | 3-hydroxyoctanoate | 1.03 | 1.02 | 2.27 | 1.09 | 0.89 |
| Fatty Acid Metabolism (Acyl Carnitine, | 3-hydroxyoctanoylcarnitine (1) | 1.28 | 1.25 | 1.50 | 1.59 | 1.50 |
| Hydroxy) | | | | | | |
| Fatty Acid Metabolism (Acyl Carnitine, | 3-hydroxyoctanoylcarnitine (2) | 1.55 | 1.58 | 1.82 | 1.59 | 2.30 |
| Hydroxy) | | | | | | |
| Fatty Acid Metabolism (Acyl Glycine) | 3-hydroxyoctanoylglycine | 1.41 | 0.70 | 0.97 | 2.44 | 1.07 |
| Fatty Acid Metabolism (Acyl Carnitine, | 3-hydroxyoleoylcarnitine | 1.40 | 1.38 | 2.17 | 1.73 | 0.88 |
| Hydroxy) | | | | | | |
| Fatty Acid, Monohydroxy | 3-hydroxypalmitate | 0.67 | 0.83 | 1.13 | 0.86 | 0.45 |
| Fatty Acid Metabolism (Acyl Carnitine, | 3-hydroxypalmitoylcarnitine | 1.25 | 1.20 | 1.56 | 1.48 | 0.98 |
| Hydroxy) | | | | | | |
| Chemical | 3-hydroxypyridine sulfate | 0.95 | 0.93 | 0.89 | 0.85 | 1.21 |
| Fatty Acid, Monohydroxy | 3-hydroxysebacate | 0.78 | 0.73 | 1.42 | 1.08 | 0.92 |
| Fatty Acid, Monohydroxy | 3-hydroxystearate | 0.52 | 0.53 | 0.69 | 0.54 | 0.35 |
| Fatty Acid, Monohydroxy | 3-hydroxysuberate | 0.68 | 0.55 | 0.61 | 0.86 | 0.73 |
| Food Component/Plant | 3-indoleglyoxylic acid | 0.64 | 0.72 | 0.96 | 0.62 | 0.75 |
| Tryptophan Metabolism | 3-indoxyl sulfate | 0.68 | 1.24 | 0.95 | 0.94 | 316.97 |
| Benzoate Metabolism | 3-methoxycatechol sulfate (1) | 2.57 | 0.55 | 1.80 | 0.93 | 1.77 |
| Benzoate Metabolism | 3-methoxycatechol sulfate (2) | 2.10 | 0.73 | 1.32 | 0.65 | 5.73 |
| Tyrosine Metabolism | 3-methoxytyrosine | 0.75 | 0.75 | 0.90 | 0.91 | 0.94 |
| Benzoate Metabolism | 3-methyl catechol sulfate (1) | 1.01 | 0.94 | 1.14 | 0.65 | 5.72 |
| Leucine, Isoleucine and Valine Metabolism | 3-methyl-2-oxobutyrate | 0.92 | 0.96 | 1.08 | 1.05 | 0.97 |
| Leucine, Isoleucine and Valine Metabolism | 3-methyl-2-oxovalerate | 0.93 | 0.98 | 1.14 | 1.11 | 1.03 |
| Leucine, Isoleucine and Valine Metabolism | 3-methylcrotonylglycine | 0.68 | 0.70 | 0.57 | 1.11 | 0.66 |
| Pyrimidine Metabolism, Cytidine containing | 3-methylcytidine | 1.09 | 0.96 | 0.99 | 0.85 | 0.64 |
| Leucine, Isoleucine and Valine Metabolism | 3-methylglutaconate | 0.77 | 0.84 | 0.91 | 0.94 | 0.95 |
| Fatty Acid, Dicarboxylate | 3-methylglutarate/2-methylglutarate | 1.17 | 0.77 | 0.76 | 0.87 | 1.15 |
| Histidine Metabolism | 3-methylhistidine | 1.26 | 1.37 | 1.31 | 1.05 | 1.23 |
| Benzoate Metabolism | 3-phenylpropionate (hydrocinnamate) | 1.29 | 0.93 | 1.53 | 3.48 | 109.01 |
| Glycolysis, Gluconeogenesis, and Pyruvate | 3-phosphoglycerate | 1.57 | 1.18 | 1.55 | 3.61 | 0.62 |
| Metabolism | | | | | | |
| Pyrimidine Metabolism, Uracil containing | 3-ureidopropionate | 1.29 | 1.44 | 1.08 | 1.31 | 1.99 |
| Fatty Acid, Dihydroxy | 3,4-dihydroxybutyrate | 1.11 | 1.07 | 1.14 | 1.02 | 1.16 |
| Tyrosine Metabolism | 3,4-dihydroxyphenylacetate sulfate | 1.05 | 0.84 | 1.17 | 0.99 | 0.84 |
| Chemical | 3,5-dichloro-2,6-dihydroxybenzoic acid | 0.70 | 1.45 | 1.10 | 1.06 | 1.11 |
| Chemical | 4-acetamidobenzoate | 0.84 | 0.96 | 1.07 | 0.66 | 2.84 |
| Polyamine Metabolism | 4-acetamidobutanoate | 1.13 | 1.19 | 1.23 | 0.98 | 0.98 |
| Food Component/Plant | 4-acetylcatechol sulfate (1) | 0.76 | 1.52 | 0.70 | 0.83 | 10.96 |
| Benzoate Metabolism | 4-acetylphenol sulfate | 1.01 | 0.94 | 0.98 | 0.99 | 2.81 |
| Benzoate Metabolism | 4-allylcatechol sulfate | 1.00 | 1.20 | 1.00 | 1.00 | 13.74 |
| Food Component/Plant | 4-allylphenol sulfate | 0.41 | 1.17 | 0.79 | 0.59 | 1.58 |
| Chemical | 4-chlorobenzoic acid | 0.98 | 1.23 | 1.19 | 1.61 | 1.09 |
| Sterol | 4-cholesten-3-one | 0.96 | 1.00 | 1.17 | 0.87 | 0.96 |
| Benzoate Metabolism | 4-ethylcatechol sulfate | 0.92 | 0.92 | 0.92 | 0.92 | 196.36 |
| Benzoate Metabolism | 4-ethylphenylsulfate | 0.98 | 0.98 | 0.98 | 0.98 | 58.21 |
| Guanidino and Acetamido Metabolism | 4-guanidinobutanoate | 0.74 | 0.68 | 0.76 | 0.99 | 1.53 |
| Fatty Acid, Monohydroxy | 4-hydroxybutyrate (GHB) | 1.03 | 0.96 | 1.17 | 1.63 | 0.87 |
| Food Component/Plant | 4-hydroxycinnamate | 0.64 | 0.93 | 0.95 | 0.73 | 0.83 |
| Tyrosine Metabolism | 4-hydroxycinnamate sulfate | 0.79 | 0.75 | 0.77 | 0.85 | 0.96 |
| Glutamate Metabolism | 4-hydroxyglutamate | 0.73 | 0.62 | 0.64 | 0.90 | 1.31 |
| Benzoate Metabolism | 4-hydroxyhippurate | 1.05 | 0.87 | 0.84 | 1.08 | 1.00 |
| Phenylalanine Metabolism | 4-hydroxyphenylacetate | 1.02 | 2.54 | 1.22 | 1.01 | 1.91 |
| Tyrosine Metabolism | 4-hydroxyphenylacetate sulfate | 0.97 | 2.73 | 0.88 | 0.74 | 1.54 |
| Tyrosine Metabolism | 4-hydroxyphenylpyruvate | 0.94 | 1.49 | 1.70 | 1.16 | 2.16 |
| Histidine Metabolism | 4-imidazoleacetate | 0.89 | 1.23 | 0.78 | 0.91 | 1.10 |
| Leucine, Isoleucine and Valine Metabolism | 4-methyl-2-oxopentanoate | 0.91 | 0.96 | 1.10 | 1.08 | 1.09 |
| Chemical | 4-methylbenzenesulfonate | 1.25 | 0.81 | 0.49 | 0.40 | 0.39 |
| Benzoate Metabolism | 4-methylcatechol sulfate | 4.58 | 1.20 | 4.94 | 0.64 | 24.63 |
| Fatty Acid Metabolism (Acyl Glycine) | 4-methylhexanoylglycine | 0.84 | 0.60 | 0.55 | 1.51 | 1.24 |
| Benzoate Metabolism | 4-vinylcatechol sulfate | 0.49 | 0.61 | 0.69 | 0.52 | 1.93 |
| Food Component/Plant | 4-vinylguaiacol glucuronide | 0.83 | 0.95 | 0.99 | 0.86 | 1.12 |
| Food Component/Plant | 4-vinylguaiacol sulfate | 1.11 | 0.64 | 0.79 | 0.75 | 1.66 |
| Benzoate Metabolism | 4-vinylphenol sulfate | 0.84 | 1.16 | 1.31 | 0.71 | 1.94 |
| Lysine Metabolism | 5-(galactosylhydroxy)-L-lysine | 0.79 | 0.80 | 0.85 | 0.87 | 0.68 |
| Lysine Metabolism | 5-aminovalerate | 1.95 | 3.23 | 1.42 | 1.63 | 1.73 |
| Medium Chain Fatty Acid | 5-dodecenoate (12:1n7) | 1.04 | 0.82 | 1.56 | 1.11 | 0.53 |
| Fatty Acid Metabolism (Acyl Carnitine, | 5-dodecenoylcarnitine (C12:1) | 1.25 | 1.05 | 1.89 | 1.19 | 1.83 |
| Monounsaturated) | | | | | | |
| Eicosanoid | 5-HETE | 0.78 | 0.85 | 0.82 | 0.61 | 0.34 |
| Chemical | 5-hydroxy-2-methylpyridine sulfate | 1.29 | 0.86 | 1.24 | 0.79 | 2.17 |
| Fatty Acid, Monohydroxy | 5-hydroxyhexanoate | 1.02 | 1.07 | 1.35 | 1.29 | 0.95 |
| Tryptophan Metabolism | 5-hydroxyindoleacetate | 1.02 | 1.18 | 1.18 | 0.97 | 0.81 |
| Lysine Metabolism | 5-hydroxylysine | 0.96 | 1.00 | 0.91 | 1.10 | 1.21 |
| Pyrimidine Metabolism, Cytidine containing | 5-methyl-2′-deoxycytidine | 1.10 | 0.90 | 1.06 | 1.04 | 2.02 |
| Pyrimidine Metabolism, Cytidine containing | 5-methylcytidine | 1.06 | 1.00 | 0.99 | 1.04 | 1.29 |
| Polyamine Metabolism | 5-methylthioadenosine (MTA) | 1.08 | 1.20 | 1.11 | 1.27 | 0.66 |
| Methionine, Cysteine, SAM and Taurine | 5-methylthioribose** | 0.97 | 1.11 | 1.15 | 1.11 | 0.87 |
| Metabolism | | | | | | |
| Pyrimidine Metabolism, Uracil containing | 5-methyluridine (ribothymidine) | 0.94 | 0.62 | 0.84 | 0.98 | 0.93 |
| Glutathione Metabolism | 5-oxoproline | 0.84 | 0.90 | 0.86 | 0.95 | 0.73 |
| Pyrimidine Metabolism, Thymine containing | 5,6-dihydrothymine | 0.76 | 0.71 | 0.87 | 1.20 | 0.64 |
| Pyrimidine Metabolism, Uracil containing | 5,6-dihydrouracil | 1.26 | 0.99 | 1.08 | 1.12 | 1.13 |
| Pyrimidine Metabolism, Uracil containing | 5,6-dihydrouridine | 1.43 | 1.87 | 1.66 | 1.49 | 1.37 |
| Chemical | 6-hydroxyindole sulfate | 1.00 | 1.00 | 1.00 | 1.00 | 2.91 |
| Lysine Metabolism | 6-oxopiperidine-2-carboxylate | 0.98 | 0.73 | 1.07 | 0.99 | 1.23 |
| Sterol | 7-alpha-hydroxy-3-oxo-4-cholestenoate (7- | 1.18 | 2.31 | 1.93 | 1.19 | 0.90 |
| | Hoca) | | | | | |
| Secondary Bile Acid Metabolism | 7-ketodeoxycholate | 4.89 | 5.30 | 32.93 | 83.25 | 2.94 |
| Purine Metabolism, Guanine containing | 7-methylguanine | 1.12 | 1.06 | 1.11 | 1.09 | 1.02 |
| Fatty Acid, Monohydroxy | 8-hydroxyoctanoate | 1.08 | 1.18 | 2.08 | 1.43 | 0.77 |
| Fatty Acid, Dihydroxy | 9,10-DiHOME | 1.23 | 0.99 | 1.74 | 1.74 | 0.98 |
| Fatty Acid Metabolism (Acyl Carnitine, Short | acetylcarnitine (C2) | 1.15 | 1.23 | 1.18 | 1.05 | 1.24 |
| Chain) | | | | | | |
| TCA Cycle | aconitate [cis or trans] | 1.05 | 1.23 | 1.20 | 1.27 | 1.12 |
| Purine Metabolism, Adenine containing | adenine | 0.68 | 0.48 | 0.66 | 0.86 | 0.63 |
| Purine Metabolism, Adenine containing | adenosine | 2.73 | 0.56 | 1.29 | 2.68 | 1.10 |
| Purine Metabolism, Adenine containing | adenosine 5′-monophosphate (AMP) | 1.01 | 0.11 | 0.67 | 3.14 | 0.63 |
| Fatty Acid, Dicarboxylate | adipate (C6-DC) | 0.99 | 0.99 | 1.51 | 1.12 | 0.64 |
| Fatty Acid Metabolism (Acyl Carnitine, | adipoylcarnitine (C6-DC) | 1.07 | 0.75 | 1.00 | 0.92 | 0.74 |
| Dicarboxylate) | | | | | | |
| Long Chain Polyunsaturated Fatty Acid (n3 | adrenate (22:4n6) | 0.81 | 1.24 | 0.88 | 1.12 | 0.44 |
| and n6) | | | | | | |
| Alanine and Aspartate Metabolism | alanine | 0.98 | 0.96 | 1.17 | 1.07 | 1.02 |
| Purine Metabolism, (Hypo)Xanthine/Inosine | allantoic acid | 0.75 | 0.85 | 0.69 | 0.68 | 0.57 |
| containing | | | | | | |
| Purine Metabolism, (Hypo)Xanthine/Inosine | allantoin | 0.99 | 1.00 | 1.05 | 1.00 | 1.04 |
| containing | | | | | | |
| Fatty Acid, Monohydroxy | alpha-hydroxycaproate | 0.80 | 0.87 | 1.78 | 2.25 | 0.78 |
| Leucine, Isoleucine and Valine Metabolism | alpha-hydroxyisocaproate | 0.41 | 0.34 | 0.64 | 0.54 | 0.43 |
| Leucine, Isoleucine and Valine Metabolism | alpha-hydroxyisovalerate | 0.33 | 0.30 | 0.34 | 0.63 | 0.29 |
| Glutamate Metabolism | alpha-ketoglutaramate* | 0.85 | 1.01 | 1.23 | 0.93 | 0.79 |
| TCA Cycle | alpha-ketoglutarate | 0.97 | 1.30 | 0.98 | 1.02 | 1.18 |
| Tocopherol Metabolism | alpha-tocopherol | 0.85 | 0.74 | 0.78 | 0.78 | 0.98 |
| Histidine Metabolism | anserine | 1.03 | 1.04 | 1.15 | 1.11 | 1.21 |
| Tryptophan Metabolism | anthranilate | 0.61 | 1.15 | 0.98 | 0.79 | 1.27 |
| Pentose Metabolism | arabitol/xylitol | 0.80 | 0.87 | 0.87 | 0.95 | 0.97 |
| Pentose Metabolism | arabonate/xylonate | 0.89 | 0.82 | 0.87 | 0.76 | 0.75 |
| Long Chain Saturated Fatty Acid | arachidate (20:0) | 0.71 | 1.04 | 0.94 | 1.31 | 0.58 |
| Long Chain Polyunsaturated Fatty Acid (n3 | arachidonate (20:4n6) | 0.67 | 0.95 | 0.83 | 0.78 | 0.41 |
| and n6) | | | | | | |
| Endocannabinoid | arachidonoyl ethanolamide | 0.90 | 0.85 | 0.98 | 1.06 | 0.93 |
| Fatty Acid Metabolism (Acyl Carnitine, | arachidonoylcarnitine (C20:4) | 1.33 | 1.22 | 1.74 | 1.44 | 0.77 |
| Polyunsaturated) | | | | | | |
| Fatty Acid Metabolism (Acyl Choline) | arachidonoylcholine | 0.94 | 0.89 | 0.81 | 0.72 | 0.85 |
| Urea cycle; Arginine and Proline Metabolism | argininate* | 0.66 | 0.64 | 0.64 | 0.93 | 0.78 |
| Urea cycle; Arginine and Proline Metabolism | arginine | 1.01 | 1.09 | 0.97 | 1.20 | 0.90 |
| Urea cycle; Arginine and Proline Metabolism | argininosuccinate | 0.99 | 1.15 | 1.21 | 1.41 | 0.74 |
| Ascorbate and Aldarate Metabolism | ascorbate (Vitamin C) | 0.26 | 0.32 | 0.58 | 0.34 | 1.17 |
| Ascorbate and Aldarate Metabolism | ascorbic acid 2-sulfate | 1.12 | 1.21 | 1.14 | 0.82 | 0.70 |
| Ascorbate and Aldarate Metabolism | ascorbic acid 3-sulfate* | 0.92 | 0.83 | 0.85 | 0.66 | 0.67 |
| Alanine and Aspartate Metabolism | asparagine | 0.98 | 1.02 | 1.10 | 1.14 | 1.10 |
| Alanine and Aspartate Metabolism | aspartate | 0.77 | 0.77 | 0.84 | 0.76 | 1.47 |
| Fatty Acid, Dicarboxylate | azelate (C9-DC) | 1.16 | 1.15 | 1.99 | 1.46 | 0.76 |
| Dihydrosphingomyelins | behenoyl dihydrosphingomyelin (d18:0/22:0)* | 1.25 | 1.06 | 0.76 | 1.40 | 1.50 |
| Sphingomyelins | behenoyl sphingomyelin (d18:1/22:0)* | 1.00 | 0.81 | 0.76 | 1.19 | 1.16 |
| Fatty Acid Metabolism (Acyl Carnitine, Long | behenoylcarnitine (C22)* | 1.17 | 1.01 | 1.01 | 1.32 | 0.96 |
| Chain Saturated) | | | | | | |
| Benzoate Metabolism | benzoate | 0.93 | 1.21 | 1.04 | 1.76 | 1.56 |
| Pyrimidine Metabolism, Uracil containing | beta-alanine | 1.29 | 1.21 | 0.95 | 1.38 | 1.12 |
| Leucine, Isoleucine and Valine Metabolism | beta-hydroxyisovalerate | 1.01 | 0.97 | 1.21 | 1.24 | 0.76 |
| Leucine, Isoleucine and Valine Metabolism | beta-hydroxyisovaleroylcarnitine | 1.05 | 1.02 | 1.06 | 0.95 | 0.90 |
| Primary Bile Acid Metabolism | beta-muricholate | 3.85 | 5.20 | 15.92 | 53.03 | 0.47 |
| Sterol | beta-sitosterol | 0.91 | 0.75 | 0.77 | 0.91 | 1.41 |
| Glycine, Serine and Threonine Metabolism | betaine | 1.21 | 1.29 | 1.23 | 1.12 | 1.10 |
| Food Component/Plant | betonicine | 0.84 | 1.02 | 1.01 | 1.03 | 1.10 |
| Hemoglobin and Porphyrin Metabolism | bilirubin (Z,Z) | 0.45 | 0.54 | 1.04 | 0.56 | 2.10 |
| Partially Characterized Molecules | bilirubin degradation product, C17H18N2O4 | 0.89 | 0.93 | 1.19 | 1.04 | 0.95 |
| | (1)** | | | | | |
| Partially Characterized Molecules | bilirubin degradation product, C17H18N2O4 | 0.87 | 0.88 | 1.10 | 1.03 | 1.02 |
| | (2)** | | | | | |
| Partially Characterized Molecules | bilirubin degradation product, C17H18N2O4 | 0.83 | 0.82 | 1.07 | 0.86 | 0.98 |
| | (3)** | | | | | |
| Partially Characterized Molecules | bilirubin degradation product, C17H20N2O5 | 0.86 | 0.78 | 1.11 | 0.77 | 0.98 |
| | (1)** | | | | | |
| Partially Characterized Molecules | bilirubin degradation product, C17H20N2O5 | 0.90 | 0.78 | 1.11 | 0.74 | 0.96 |
| | (2)** | | | | | |
| Hemoglobin and Porphyrin Metabolism | biliverdin | 1.01 | 1.05 | 1.06 | 1.02 | 0.98 |
| Tetrahydrobiopterin Metabolism | biopterin | 0.96 | 0.99 | 1.07 | 1.07 | 1.25 |
| Fatty Acid, Dicarboxylate | branched chain 14:0 dicarboxylic acid** | 0.84 | 1.04 | 1.08 | 1.16 | 3.75 |
| Partially Characterized Molecules | branched-chain, straight-chain, or cyclopropyl | 0.66 | 1.01 | 1.21 | 1.35 | 0.53 |
| | 10:1 fatty acid (1)* | | | | | |
| Partially Characterized Molecules | branched-chain, straight-chain, or cyclopropyl | 1.28 | 1.35 | 1.80 | 1.57 | 0.58 |
| | 10:1 fatty acid (3)* | | | | | |
| Partially Characterized Molecules | branched-chain, straight-chain, or cyclopropyl | 1.00 | 0.79 | 1.41 | 0.84 | 1.34 |
| | 12:1 fatty acid* | | | | | |
| Short Chain Fatty Acid | butyrate/isobutyrate (4:0) | 1.00 | 1.24 | 1.45 | 1.13 | 2.41 |
| Fatty Acid Metabolism (also BCAA | butyrylcarnitine (C4) | 1.25 | 1.38 | 1.04 | 0.80 | 5.44 |
| Metabolism) | | | | | | |
| Fatty Acid Metabolism (also BCAA | butyrylglycine | 0.72 | 0.58 | 0.42 | 0.79 | 4.62 |
| Metabolism) | | | | | | |
| Tryptophan Metabolism | C-glycosyltryptophan | 1.28 | 1.30 | 1.33 | 1.28 | 1.10 |
| Food Component/Plant | caffeic acid sulfate | 0.68 | 1.20 | 0.88 | 0.68 | 0.76 |
| Sterol | campesterol | 0.92 | 0.79 | 0.85 | 0.92 | 1.14 |
| Medium Chain Fatty Acid | caprate (10:0) | 1.10 | 1.04 | 1.57 | 1.21 | 0.87 |
| Medium Chain Fatty Acid | caproate (6:0) | 0.96 | 0.99 | 1.34 | 1.16 | 3.62 |
| Medium Chain Fatty Acid | caprylate (8:0) | 1.05 | 1.05 | 1.54 | 1.28 | 2.49 |
| Glutamate Metabolism | carboxyethyl-GABA | 0.94 | 1.03 | 1.07 | 1.17 | 0.21 |
| Carnitine Metabolism | carnitine | 1.23 | 1.35 | 1.27 | 1.08 | 0.93 |
| Histidine Metabolism | carnosine | 1.14 | 0.77 | 1.14 | 0.63 | 1.18 |
| Vitamin A Metabolism | carotene diol (1) | 1.17 | 1.28 | 1.53 | 0.82 | 1.12 |
| Vitamin A Metabolism | carotene diol (2) | 0.86 | 1.30 | 1.56 | 0.56 | 0.99 |
| Tyrosine Metabolism | catechol glucuronide | 1.00 | 1.00 | 1.00 | 1.00 | 1.26 |
| Benzoate Metabolism | catechol sulfate | 1.16 | 0.83 | 1.19 | 0.74 | 167.54 |
| Ceramides | ceramide (d16:1/24:1, d18:1/22:1)* | 1.19 | 1.04 | 0.93 | 1.58 | 1.44 |
| Ceramides | ceramide (d18:1/20:0, d16:1/22:0, d20:1/18:0)* | 1.03 | 0.64 | 0.81 | 0.87 | 1.34 |
| Ceramides | ceramide (d18:2/24:1, d18:1/24:2)* | 1.12 | 1.08 | 0.94 | 1.13 | 1.15 |
| Primary Bile Acid Metabolism | chenodeoxycholate | 2.99 | 5.14 | 6.81 | 6.61 | 0.52 |
| Inositol Metabolism | chiro-inositol | 1.63 | 2.10 | 2.08 | 1.81 | 1.09 |
| Primary Bile Acid Metabolism | cholate | 16.37 | 27.62 | 35.33 | 28.37 | 1.70 |
| Sterol | cholesterol | 0.88 | 0.89 | 0.92 | 0.96 | 0.99 |
| Sterol | cholesterol sulfate | 0.97 | 1.13 | 1.08 | 1.10 | 0.43 |
| Phospholipid Metabolism | choline | 0.91 | 0.87 | 0.90 | 1.09 | 1.08 |
| Phospholipid Metabolism | choline phosphate | 0.99 | 0.77 | 0.87 | 0.88 | 0.79 |
| Food Component/Plant | cinnamate | 0.91 | 0.95 | 0.89 | 1.35 | 9.54 |
| Food Component/Plant | cinnamoylglycine | 1.03 | 0.73 | 0.68 | 7.87 | 75.87 |
| Fatty Acid, Branched | cis-3,4-methyleneheptanoate | 1.21 | 1.23 | 3.26 | 1.67 | 1.01 |
| Fatty Acid Metabolism (Acyl Carnitine, | cis-3,4-methyleneheptanoylcarnitine | 1.31 | 1.05 | 1.81 | 1.17 | 1.13 |
| Medium Chain) | | | | | | |
| Medium Chain Fatty Acid | cis-4-decenoate (10:1n6)* | 0.89 | 1.01 | 1.78 | 1.22 | 0.58 |
| Fatty Acid Metabolism (Acyl Carnitine, | cis-4-decenoylcarnitine (C10:1) | 1.37 | 1.21 | 1.81 | 1.34 | 1.15 |
| Monounsaturated) | | | | | | |
| TCA Cycle | citrate | 0.99 | 1.34 | 1.21 | 1.38 | 1.07 |
| Fatty Acid, Branched | citronellic acid | 0.82 | 0.88 | 0.76 | 1.13 | 0.76 |
| Urea cycle; Arginine and Proline Metabolism | citrulline | 0.96 | 1.27 | 1.10 | 1.17 | 0.79 |
| Corticosteroids | corticosterone | 1.26 | 2.29 | 1.80 | 1.33 | 1.21 |
| Creatine Metabolism | creatine | 1.02 | 1.25 | 1.16 | 1.23 | 1.10 |
| Creatine Metabolism | creatinine | 0.96 | 1.10 | 1.03 | 1.17 | 1.02 |
| Methionine, Cysteine, SAM and Taurine | cystathionine | 1.04 | 1.09 | 1.33 | 1.02 | 1.28 |
| Metabolism | | | | | | |
| Methionine, Cysteine, SAM and Taurine | cysteine | 1.00 | 1.26 | 1.11 | 1.41 | 1.34 |
| Metabolism | | | | | | |
| Methionine, Cysteine, SAM and Taurine | cysteine s-sulfate | 1.13 | 1.02 | 1.02 | 0.89 | 1.23 |
| Metabolism | | | | | | |
| Methionine, Cysteine, SAM and Taurine | cysteine sulfinic acid | 0.85 | 0.88 | 0.87 | 0.83 | 1.48 |
| Metabolism | | | | | | |
| Glutathione Metabolism | cysteine-glutathione disulfide | 1.09 | 1.02 | 0.92 | 0.88 | 2.87 |
| Glutathione Metabolism | cysteinylglycine disulfide* | 1.49 | 1.24 | 1.00 | 0.82 | 2.11 |
| Methionine, Cysteine, SAM and Taurine | cystine | 1.16 | 1.23 | 1.09 | 0.98 | 2.14 |
| Metabolism | | | | | | |
| Pyrimidine Metabolism, Cytidine containing | cytidine | 1.03 | 1.06 | 0.98 | 1.08 | 0.97 |
| Pyrimidine Metabolism, Cytidine containing | cytidine 5′-monophosphate (5′-CMP) | 1.35 | 1.05 | 1.34 | 1.68 | 0.74 |
| Pyrimidine Metabolism, Cytidine containing | cytosine | 0.93 | 1.04 | 0.86 | 1.06 | 1.25 |
| Food Component/Plant | daidzein | 0.81 | 1.01 | 0.87 | 0.96 | 0.93 |
| Food Component/Plant | daidzein 7-O-glucuronide | 0.94 | 1.23 | 1.23 | 1.10 | 1.63 |
| Food Component/Plant | daidzein sulfate (1) | 0.41 | 0.55 | 0.85 | 0.87 | 0.77 |
| Food Component/Plant | daidzein sulfate (2) | 0.66 | 1.02 | 1.05 | 1.16 | 0.79 |
| Fatty Acid, Dicarboxylate | decadienedioic acid (C10:2-DC)** | 1.26 | 1.11 | 3.05 | 0.97 | 0.88 |
| Fatty Acid Metabolism (Acyl Carnitine, | decanoylcarnitine (C10) | 1.25 | 1.05 | 1.73 | 1.48 | 2.19 |
| Medium Chain) | | | | | | |
| Carnitine Metabolism | deoxycarnitine | 0.84 | 0.77 | 0.84 | 0.92 | 0.68 |
| Secondary Bile Acid Metabolism | deoxycholate | 1.00 | 1.00 | 1.00 | 1.00 | 4.29 |
| Chemical | dibutyl sulfosuccinate | 0.91 | 0.50 | 0.85 | 1.17 | 1.33 |
| Long Chain Polyunsaturated Fatty Acid (n3 | dihomo-linoleate (20:2n6) | 0.80 | 1.47 | 1.09 | 1.18 | 0.46 |
| and n6) | | | | | | |
| Long Chain Polyunsaturated Fatty Acid (n3 | dihomo-linolenate (20:3n3 or n6) | 0.77 | 1.26 | 0.90 | 0.99 | 0.42 |
| and n6) | | | | | | |
| Fatty Acid Metabolism (Acyl Carnitine, | dihomo-linolenoylcarnitine (C20:3n3 or 6)* | 1.21 | 1.08 | 1.68 | 1.42 | 0.84 |
| Polyunsaturated) | | | | | | |
| Fatty Acid Metabolism (Acyl Carnitine, | dihomo-linoleoylcarnitine (C20:2)* | 1.36 | 1.06 | 1.46 | 1.64 | 1.06 |
| Polyunsaturated) | | | | | | |
| Tetrahydrobiopterin Metabolism | dihydrobiopterin | 0.99 | 0.82 | 0.97 | 0.76 | 1.27 |
| Food Component/Plant | dihydrocaffeate sulfate (2) | 0.68 | 15.80 | 0.30 | 0.63 | 1.31 |
| Food Component/Plant | dihydroferulate | 0.38 | 0.26 | 0.26 | 0.78 | 0.62 |
| Food Component/Plant | dihydroferulic acid sulfate | 0.16 | 0.10 | 0.08 | 0.55 | 0.29 |
| Pyrimidine Metabolism, Orotate containing | dihydroorotate | 1.29 | 0.95 | 0.78 | 0.83 | 1.26 |
| Glycolysis, Gluconeogenesis, and Pyruvate | dihydroxyacetone phosphate (DHAP) | 1.45 | 1.56 | 2.08 | 1.73 | 0.90 |
| Metabolism | | | | | | |
| Chemical | dimethyl sulfone | 0.84 | 0.79 | 0.80 | 0.65 | 4.06 |
| Urea cycle; Arginine and Proline Metabolism | dimethylarginine (SDMA + ADMA) | 0.98 | 0.98 | 0.98 | 1.05 | 0.67 |
| Glycine, Serine and Threonine Metabolism | dimethylglycine | 0.94 | 0.94 | 0.94 | 0.88 | 0.81 |
| Fatty Acid, Dicarboxylate | dimethylmalonic acid | 0.77 | 1.14 | 1.06 | 1.23 | 0.77 |
| Long Chain Polyunsaturated Fatty Acid (n3 | docosadienoate (22:2n6) | 0.80 | 1.86 | 1.01 | 1.67 | 0.58 |
| and n6) | | | | | | |
| Fatty Acid, Dicarboxylate | docosadioate (C22-DC) | 0.74 | 0.91 | 1.20 | 1.49 | 0.84 |
| Long Chain Polyunsaturated Fatty Acid (n3 | docosahexaenoate (DHA; 22:6n3) | 0.83 | 1.10 | 0.92 | 0.98 | 0.51 |
| and n6) | | | | | | |
| Fatty Acid Metabolism (Acyl Choline) | docosahexaenoylcholine | 0.96 | 0.81 | 0.75 | 0.68 | 1.18 |
| Long Chain Polyunsaturated Fatty Acid (n3 | docosapentaenoate (n3 DPA; 22:5n3) | 0.77 | 1.33 | 0.95 | 1.19 | 0.69 |
| and n6) | | | | | | |
| Long Chain Polyunsaturated Fatty Acid (n3 | docosapentaenoate (n6 DPA; 22:5n6) | 0.79 | 0.86 | 0.84 | 1.03 | 0.31 |
| and n6) | | | | | | |
| Fatty Acid Metabolism (Acyl Carnitine, | docosapentaenoylcarnitine (C22:5n3)* | 1.32 | 0.89 | 1.94 | 1.55 | 1.08 |
| Polyunsaturated) | | | | | | |
| Long Chain Polyunsaturated Fatty Acid (n3 | docosatrienoate (22:3n3) | 0.77 | 1.90 | 1.07 | 1.40 | 0.49 |
| and n6) | | | | | | |
| Long Chain Polyunsaturated Fatty Acid (n3 | docosatrienoate (22:3n6)* | 0.95 | 1.75 | 0.81 | 1.70 | 0.38 |
| and n6) | | | | | | |
| Fatty Acid, Dicarboxylate | dodecadienoate (12:2)* | 1.00 | 0.97 | 1.85 | 1.27 | 0.55 |
| Fatty Acid, Dicarboxylate | dodecanedioate (C12-DC) | 1.22 | 1.22 | 2.24 | 1.46 | 0.82 |
| Fatty Acid, Dicarboxylate | dodecenedioate (C12:1-DC)* | 0.83 | 1.42 | 1.84 | 1.87 | 1.36 |
| Tyrosine Metabolism | dopamine 3-O-sulfate | 0.97 | 1.05 | 1.26 | 0.86 | 0.82 |
| Chemical | ectoine | 0.90 | 1.31 | 0.99 | 1.28 | 1.00 |
| Fatty Acid, Dicarboxylate | eicosanedioate (C20-DC) | 0.76 | 0.75 | 1.04 | 1.47 | 0.85 |
| Long Chain Polyunsaturated Fatty Acid (n3 | eicosapentaenoate (EPA; 20:5n3) | 0.68 | 0.80 | 0.95 | 0.83 | 0.43 |
| and n6) | | | | | | |
| Fatty Acid, Dicarboxylate | eicosenedioate (C20:1-DC)* | 1.07 | 1.19 | 1.90 | 1.03 | 1.38 |
| Long Chain Monounsaturated Fatty Acid | eicosenoate (20:1) | 0.77 | 1.09 | 1.09 | 1.63 | 0.56 |
| Fatty Acid Metabolism (Acyl Carnitine, | eicosenoylcarnitine (C20:1)* | 1.34 | 1.11 | 1.46 | 2.11 | 1.25 |
| Monounsaturated) | | | | | | |
| Food Component/Plant | enterolactone sulfate | 1.00 | 1.00 | 1.00 | 1.00 | 1.83 |
| Food Component/Plant | equol glucuronide | 1.00 | 1.00 | 1.00 | 1.00 | 1.46 |
| Food Component/Plant | equol sulfate | 1.00 | 1.00 | 1.00 | 1.00 | 1.36 |
| Food Component/Plant | ergothioneine | 1.06 | 1.09 | 0.95 | 1.24 | 0.81 |
| Long Chain Monounsaturated Fatty Acid | erucate (22:1n9) | 0.80 | 1.32 | 1.27 | 2.02 | 0.71 |
| Food Component/Plant | erythritol | 1.08 | 1.18 | 1.01 | 1.22 | 1.22 |
| Aminosugar Metabolism | erythronate* | 1.01 | 0.95 | 0.97 | 0.99 | 1.00 |
| Food Component/Plant | ethyl alpha-glucopyranoside | 0.82 | 1.39 | 1.00 | 1.55 | 0.91 |
| Food Component/Plant | ethyl beta-glucopyranoside | 1.08 | 1.64 | 1.35 | 1.44 | 0.81 |
| Leucine, Isoleucine and Valine Metabolism | ethylmalonate | 1.08 | 1.29 | 1.15 | 0.83 | 4.92 |
| Food Component/Plant | ferulate | 0.77 | 1.29 | 0.83 | 0.75 | 0.84 |
| Food Component/Plant | ferulic acid 4-sulfate | 0.83 | 0.89 | 0.75 | 0.80 | 0.99 |
| Histidine Metabolism | formiminoglutamate | 0.64 | 0.69 | 1.16 | 0.73 | 1.49 |
| Fructose, Mannose and Galactose Metabolism | fructose | 0.93 | 0.73 | 0.69 | 0.70 | 0.95 |
| Lysine Metabolism | fructosyllysine | 0.77 | 0.90 | 0.72 | 1.13 | 0.47 |
| TCA Cycle | fumarate | 1.02 | 1.04 | 0.96 | 1.11 | 1.04 |
| Fructose, Mannose and Galactose Metabolism | galactonate | 1.65 | 1.84 | 2.10 | 1.49 | 0.95 |
| Gamma-glutamyl Amino Acid | gamma-glutamyl-alpha-lysine | 0.95 | 0.92 | 1.00 | 1.00 | 1.68 |
| Gamma-glutamyl Amino Acid | gamma-glutamyl-epsilon-lysine | 0.69 | 0.74 | 1.01 | 0.91 | 0.83 |
| Gamma-glutamyl Amino Acid | gamma-glutamylalanine | 0.95 | 0.96 | 1.37 | 2.42 | 1.53 |
| Gamma-glutamyl Amino Acid | gamma-glutamylcitrulline* | 0.76 | 0.93 | 1.08 | 0.77 | 1.12 |
| Gamma-glutamyl Amino Acid | gamma-glutamylglutamate | 1.01 | 1.05 | 0.87 | 0.97 | 2.89 |
| Gamma-glutamyl Amino Acid | gamma-glutamylglutamine | 0.99 | 1.05 | 0.96 | 1.11 | 2.16 |
| Gamma-glutamyl Amino Acid | gamma-glutamylglycine | 0.90 | 0.91 | 0.86 | 0.96 | 2.07 |
| Gamma-glutamyl Amino Acid | gamma-glutamylhistidine | 0.94 | 0.98 | 1.00 | 0.81 | 1.85 |
| Gamma-glutamyl Amino Acid | gamma-glutamylisoleucine* | 0.80 | 0.96 | 0.89 | 0.96 | 1.57 |
| Gamma-glutamyl Amino Acid | gamma-glutamylleucine | 0.80 | 0.91 | 0.93 | 0.92 | 1.87 |
| Gamma-glutamyl Amino Acid | gamma-glutamylmethionine | 0.84 | 0.86 | 0.94 | 0.76 | 2.57 |
| Gamma-glutamyl Amino Acid | gamma-glutamylphenylalanine | 0.83 | 1.07 | 1.06 | 1.02 | 1.62 |
| Gamma-glutamyl Amino Acid | gamma-glutamylserine | 1.01 | 0.96 | 1.05 | 0.90 | 1.73 |
| Gamma-glutamyl Amino Acid | gamma-glutamylthreonine | 0.97 | 1.12 | 1.09 | 1.06 | 1.88 |
| Gamma-glutamyl Amino Acid | gamma-glutamyltryptophan | 0.91 | 1.04 | 1.14 | 0.91 | 2.07 |
| Gamma-glutamyl Amino Acid | gamma-glutamyltyrosine | 0.85 | 0.94 | 0.96 | 0.88 | 1.82 |
| Gamma-glutamyl Amino Acid | gamma-glutamylvaline | 0.80 | 0.91 | 0.90 | 0.95 | 1.71 |
| Tocopherol Metabolism | gamma-tocopherol/beta-tocopherol | 0.90 | 0.75 | 0.79 | 0.95 | 1.11 |
| Food Component/Plant | genistein | 0.71 | 1.05 | 0.95 | 1.17 | 1.03 |
| Food Component/Plant | genistein glucuronide* | 0.85 | 1.50 | 1.55 | 1.37 | 1.65 |
| Food Component/Plant | genistein sulfate* | 0.59 | 1.18 | 1.22 | 1.28 | 0.68 |
| Tyrosine Metabolism | gentisate | 0.88 | 1.04 | 0.91 | 0.82 | 2.25 |
| Food Component/Plant | gluconate | 1.19 | 1.04 | 1.13 | 1.04 | 1.22 |
| Glycolysis, Gluconeogenesis, and Pyruvate | glucose | 0.95 | 0.88 | 0.83 | 0.87 | 0.97 |
| Metabolism | | | | | | |
| Glycolysis, Gluconeogenesis, and Pyruvate | glucose 6-phosphate | 1.37 | 1.05 | 1.61 | 3.27 | 0.62 |
| Metabolism | | | | | | |
| Aminosugar Metabolism | glucuronate | 0.98 | 1.02 | 0.99 | 0.89 | 2.07 |
| Partially Characterized Molecules | glucuronide of C14H22O4 (1)* | 0.87 | 0.88 | 1.64 | 1.01 | 3.44 |
| Glutamate Metabolism | glutamate | 1.01 | 0.99 | 1.05 | 1.11 | 1.40 |
| Glutamate Metabolism | glutamine | 0.95 | 0.95 | 0.93 | 1.07 | 1.03 |
| Partially Characterized Molecules | glutamine_degradant* | 0.88 | 1.28 | 1.98 | 1.07 | 0.98 |
| Fatty Acid, Dicarboxylate | glutarate (C5-DC) | 0.80 | 0.75 | 0.81 | 0.74 | 0.87 |
| Lysine Metabolism | glutarylcarnitine (C5-DC) | 1.26 | 0.99 | 1.15 | 0.80 | 0.37 |
| Glutathione Metabolism | glutathione, oxidized (GSSG) | 1.00 | 1.00 | 1.04 | 0.99 | 2.40 |
| Glycolysis, Gluconeogenesis, and Pyruvate | glycerate | 1.00 | 0.98 | 1.04 | 1.00 | 1.12 |
| Metabolism | | | | | | |
| Glycerolipid Metabolism | glycerol | 0.97 | 0.91 | 1.06 | 1.14 | 0.89 |
| Glycerolipid Metabolism | glycerol 3-phosphate | 0.87 | 0.74 | 0.91 | 1.01 | 1.83 |
| Phospholipid Metabolism | glycerophosphoethanolamine | 0.92 | 0.90 | 0.79 | 0.88 | 0.58 |
| Glycerolipid Metabolism | glycerophosphoglycerol | 0.81 | 0.72 | 0.94 | 0.93 | 1.57 |
| Phospholipid Metabolism | glycerophosphoinositol* | 0.70 | 0.66 | 0.64 | 0.45 | 0.57 |
| Phospholipid Metabolism | glycerophosphorylcholine (GPC) | 0.93 | 0.91 | 0.91 | 0.96 | 0.87 |
| Phospholipid Metabolism | glycerophosphoserine* | 1.15 | 1.14 | 1.10 | 1.16 | 1.07 |
| Glycine, Serine and Threonine Metabolism | glycine | 0.90 | 0.93 | 0.84 | 1.07 | 1.04 |
| Partially Characterized Molecules | glycine conjugate of C6H10O2 (2)* | 1.24 | 0.90 | 0.75 | 1.44 | 1.09 |
| Partially Characterized Molecules | glycine conjugate of C6H10O2 (3)* | 0.96 | 0.63 | 0.92 | 1.31 | 1.76 |
| Food Component/Plant | glycitein sulfate (2) | 0.70 | 0.98 | 1.25 | 0.90 | 0.64 |
| Primary Bile Acid Metabolism | glyco-beta-muricholate** | 4.41 | 3.13 | 9.47 | 3.13 | 0.79 |
| Primary Bile Acid Metabolism | glycocholate | 6.63 | 6.25 | 15.63 | 3.56 | 0.87 |
| Chemical | glycolate (hydroxyacetate) | 0.95 | 0.80 | 1.17 | 0.90 | 1.55 |
| Hexosylceramides (HCER) | glycosyl ceramide (d16:1/24:1, d18:1/22:1)* | 1.56 | 1.26 | 0.97 | 1.56 | 0.89 |
| Hexosylceramides (HCER) | glycosyl ceramide (d18:1/20:0, d16:1/22:0)* | 1.24 | 0.79 | 0.90 | 0.93 | 1.14 |
| Hexosylceramides (HCER) | glycosyl ceramide (d18:1/23:1, d17:1/24:1)* | 1.35 | 1.20 | 1.11 | 0.77 | 0.82 |
| Hexosylceramides (HCER) | glycosyl ceramide (d18:2/24:1, d18:1/24:2)* | 1.38 | 1.13 | 0.96 | 1.02 | 1.00 |
| Hexosylceramides (HCER) | glycosyl-N-behenoyl-sphingadienine | 0.99 | 0.87 | 0.64 | 1.10 | 0.85 |
| | (d18:2/22:0)* | | | | | |
| Hexosylceramides (HCER) | glycosyl-N-behenoyl-sphingosine (d18:1/22:0)* | 1.35 | 0.99 | 0.72 | 1.14 | 1.15 |
| Hexosylceramides (HCER) | glycosyl-N-nervonoyl-sphingosine (d18:1/24:1)* | 1.36 | 1.09 | 0.97 | 0.97 | 1.08 |
| Hexosylceramides (HCER) | glycosyl-N-palmitoyl-sphingosine (d18:1/16:0) | 1.27 | 0.88 | 1.01 | 0.76 | 1.21 |
| Hexosylceramides (HCER) | glycosyl-N-stearoyl-sphingosine (d18:1/18:0) | 1.75 | 0.69 | 1.18 | 0.39 | 1.66 |
| Dipeptide | glycylvaline | 1.06 | 1.12 | 0.71 | 1.13 | 1.22 |
| Benzoate Metabolism | guaiacol sulfate | 0.44 | 0.37 | 0.61 | 0.58 | 2.95 |
| Creatine Metabolism | guanidinoacetate | 1.03 | 1.05 | 1.38 | 2.32 | 0.73 |
| Guanidino and Acetamido Metabolism | guanidinosuccinate | 0.64 | 0.48 | 0.69 | 0.68 | 0.86 |
| Purine Metabolism, Guanine containing | guanosine | 0.80 | 0.96 | 1.43 | 0.30 | 0.32 |
| Purine Metabolism, Guanine containing | guanosine 5′-monophosphate (5′-GMP) | 1.29 | 0.48 | 1.12 | 2.02 | 0.62 |
| Ascorbate and Aldarate Metabolism | gulonate* | 0.95 | 0.92 | 0.85 | 0.89 | 2.62 |
| Hemoglobin and Porphyrin Metabolism | heme | 1.22 | 1.40 | 1.14 | 1.56 | 0.53 |
| Long Chain Polyunsaturated Fatty Acid (n3 | heneicosapentaenoate (21:5n3) | 0.88 | 1.23 | 0.98 | 1.09 | 0.42 |
| and n6) | | | | | | |
| Fatty Acid, Dicarboxylate | heptadecanedioate (C17-DC) | 0.97 | 0.79 | 1.28 | 1.42 | 1.01 |
| Medium Chain Fatty Acid | heptanoate (7:0) | 1.01 | 1.03 | 1.52 | 1.11 | 1.68 |
| Fatty Acid Metabolism (Acyl Glycine) | heptanoyl glycine | 0.53 | 0.41 | 0.37 | 1.13 | 1.48 |
| Fatty Acid, Dicarboxylate | heptenedioate (C7:1-DC)* | 1.24 | 0.84 | 1.35 | 0.65 | 0.73 |
| Long Chain Polyunsaturated Fatty Acid (n3 | hexadecadienoate (16:2n6) | 0.98 | 1.10 | 1.47 | 1.38 | 0.55 |
| and n6) | | | | | | |
| Fatty Acid, Dicarboxylate | hexadecanedioate (C16-DC) | 0.91 | 0.70 | 1.29 | 1.29 | 1.06 |
| Long Chain Polyunsaturated Fatty Acid (n3 | hexadecatrienoate (16:3n3) | 1.07 | 0.96 | 1.61 | 1.38 | 0.43 |
| and n6) | | | | | | |
| Fatty Acid, Dicarboxylate | hexadecenedioate (C16:1-DC)* | 0.94 | 0.83 | 1.33 | 1.56 | 1.07 |
| Fatty Acid Metabolism (Acyl Carnitine, | hexanoylcarnitine (C6) | 1.30 | 1.38 | 1.12 | 0.74 | 16.67 |
| Medium Chain) | | | | | | |
| Fatty Acid Metabolism (Acyl Glycine) | hexanoylglycine | 0.73 | 0.69 | 0.42 | 1.06 | 3.04 |
| Endocannabinoid | hexanoyltaurine | 0.72 | 0.43 | 0.89 | 0.91 | 7.84 |
| Benzoate Metabolism | hippurate | 1.32 | 0.95 | 1.04 | 1.91 | 17.41 |
| Histidine Metabolism | histamine | 0.74 | 1.80 | 1.03 | 0.83 | 0.53 |
| Histidine Metabolism | histidine | 1.08 | 1.10 | 1.09 | 1.08 | 1.09 |
| Food Component/Plant | histidine betaine (hercynine)* | 0.94 | 1.06 | 1.20 | 1.08 | 0.57 |
| Urea cycle; Arginine and Proline Metabolism | homoarginine | 0.92 | 0.98 | 0.99 | 0.97 | 0.43 |
| Urea cycle; Arginine and Proline Metabolism | homocitrulline | 1.03 | 1.18 | 1.05 | 0.82 | 1.14 |
| Food Component/Plant | homostachydrine* | 1.10 | 1.57 | 1.25 | 1.08 | 0.74 |
| Tyrosine Metabolism | homovanillate sulfate | 0.89 | 0.70 | 1.07 | 0.93 | 1.01 |
| Histidine Metabolism | hydantoin-5-propionate | 0.65 | 0.53 | 0.46 | 0.92 | 0.68 |
| Food Component/Plant | hydroquinone beta-D-glucopyranoside | 1.12 | 0.83 | 0.98 | 0.89 | 1.29 |
| Drug - Topical Agents | hydroquinone sulfate | 0.80 | 0.89 | 0.86 | 0.68 | 5.84 |
| Lysine Metabolism | hydroxy-N6,N6,N6-trimethyllysine* | 0.95 | 1.10 | 1.00 | 1.20 | 0.68 |
| Fatty Acid, Monohydroxy | hydroxy-undecanedioate (OH—C11:0-DC)* | 1.14 | 1.04 | 1.88 | 1.26 | 0.64 |
| Alanine and Aspartate Metabolism | hydroxyasparagine** | 1.07 | 1.10 | 1.12 | 0.96 | 0.97 |
| Thiamine Metabolism | hydroxymethylpyrimidine | 0.83 | 1.01 | 0.90 | 0.90 | 0.85 |
| Sphingomyelins | hydroxypalmitoyl sphingomyelin | 0.85 | 0.94 | 1.00 | 1.02 | 0.85 |
| | (d18:1/16:0(OH))** | | | | | |
| Secondary Bile Acid Metabolism | hyocholate | 1.43 | 1.66 | 3.05 | 3.40 | 0.99 |
| Methionine, Cysteine, SAM and Taurine | hypotaurine | 1.23 | 1.82 | 1.49 | 1.79 | 2.24 |
| Metabolism | | | | | | |
| Purine Metabolism, (Hypo)Xanthine/Inosine | hypoxanthine | 1.12 | 1.55 | 2.27 | 1.21 | 0.71 |
| containing | | | | | | |
| Histidine Metabolism | imidazole lactate | 0.83 | 1.00 | 0.75 | 1.17 | 1.23 |
| Histidine Metabolism | imidazole propionate | 0.98 | 0.81 | 0.93 | 1.00 | 1.73 |
| Tryptophan Metabolism | indole-3-carboxylate | 0.91 | 0.83 | 1.14 | 0.99 | 1.02 |
| Tryptophan Metabolism | indoleacetate | 1.20 | 2.94 | 1.87 | 0.77 | 1.31 |
| Tryptophan Metabolism | indoleacetylglycine | 1.25 | 3.10 | 1.13 | 0.85 | 1.05 |
| Tryptophan Metabolism | indoleacrylate | 0.91 | 1.14 | 1.07 | 40.93 | 55.13 |
| Tryptophan Metabolism | indolelactate | 0.73 | 0.93 | 1.08 | 0.87 | 1.00 |
| Tryptophan Metabolism | indolepropionate | 1.22 | 1.11 | 1.37 | 29.57 | 171.90 |
| Tryptophan Metabolism | indolepropionylglycine | 1.00 | 1.00 | 1.00 | 1.36 | 2.24 |
| Food Component/Plant | indolin-2-one | 1.00 | 1.00 | 1.00 | 1.00 | 3.82 |
| Tryptophan Metabolism | indoxyl glucuronide | 1.00 | 1.00 | 1.00 | 1.00 | 2.31 |
| Purine Metabolism, (Hypo)Xanthine/Inosine | inosine | 1.07 | 1.50 | 2.50 | 0.86 | 0.55 |
| containing | | | | | | |
| Leucine, Isoleucine and Valine Metabolism | isobutyrylcarnitine (C4) | 1.41 | 1.22 | 1.29 | 0.97 | 1.70 |
| Fatty Acid Metabolism (Acyl Carnitine, Short | isocaproylcarnitine | 2.88 | 1.38 | 1.59 | 0.77 | 4.62 |
| Chain) | | | | | | |
| Fatty Acid Metabolism (Acyl Glycine) | isocaproylglycine | 1.26 | 0.61 | 0.52 | 1.26 | 1.14 |
| TCA Cycle | isocitrate | 0.81 | 1.09 | 1.17 | 1.15 | 1.96 |
| TCA Cycle | isocitric lactone | 1.68 | 1.62 | 1.97 | 1.19 | 1.23 |
| Leucine, Isoleucine and Valine Metabolism | isoleucine | 0.87 | 0.99 | 0.93 | 1.02 | 0.91 |
| Dipeptide Derivative | isoleucylhydroxyproline* | 0.85 | 0.82 | 0.60 | 0.89 | 0.99 |
| Leucine, Isoleucine and Valine Metabolism | isovalerate (15:0) | 0.81 | 0.78 | 1.08 | 1.14 | 3.58 |
| Leucine, Isoleucine and Valine Metabolism | isovalerylcarnitine (C5) | 0.81 | 1.00 | 0.92 | 0.74 | 2.08 |
| Leucine, Isoleucine and Valine Metabolism | isovalerylglycine | 0.58 | 0.52 | 0.44 | 0.83 | 1.36 |
| TCA Cycle | itaconate | 1.59 | 1.39 | 1.12 | 0.91 | 2.63 |
| Tryptophan Metabolism | kynurenate | 0.74 | 0.87 | 0.87 | 0.83 | 0.45 |
| Tryptophan Metabolism | kynurenine | 0.74 | 1.08 | 1.28 | 0.76 | 1.36 |
| Glycolysis, Gluconeogenesis, and Pyruvate | lactate | 1.06 | 1.13 | 0.96 | 1.05 | 1.01 |
| Metabolism | | | | | | |
| Lactosylceramides (LCER) | lactosyl-N-palmitoyl-sphingosine (d18:1/16:0) | 0.69 | 0.73 | 0.92 | 0.93 | 1.13 |
| Methionine, Cysteine, SAM and Taurine | lanthionine | 0.85 | 1.28 | 0.98 | 1.04 | 0.94 |
| Metabolism | | | | | | |
| Fatty Acid Metabolism (Acyl Carnitine, | laurylcarnitine (C12) | 1.23 | 1.09 | 1.75 | 1.43 | 1.13 |
| Medium Chain) | | | | | | |
| Leucine, Isoleucine and Valine Metabolism | leucine | 0.86 | 0.92 | 0.91 | 0.99 | 0.89 |
| Dipeptide Derivative | leucylhydroxyproline* | 0.97 | 0.84 | 0.81 | 0.92 | 0.63 |
| Sphingomyelins | lignoceroyl sphingomyelin (d18:1/24:0) | 0.93 | 0.79 | 0.80 | 0.96 | 1.05 |
| Fatty Acid Metabolism (Acyl Carnitine, Long | lignoceroylcarnitine (C24)* | 1.24 | 1.00 | 1.00 | 1.03 | 0.96 |
| Chain Saturated) | | | | | | |
| Long Chain Polyunsaturated Fatty Acid (n3 | linoleate (18:2n6) | 0.78 | 1.08 | 1.12 | 1.07 | 0.51 |
| and n6) | | | | | | |
| Long Chain Polyunsaturated Fatty Acid (n3 | linolenate [alpha or gamma; (18:3n3 or 6)] | 0.79 | 1.03 | 1.28 | 1.13 | 0.53 |
| and n6) | | | | | | |
| Fatty Acid Metabolism (Acyl Carnitine, | linolenoylcarnitine (C18:3)* | 1.30 | 1.26 | 1.77 | 1.72 | 1.00 |
| Polyunsaturated) | | | | | | |
| Endocannabinoid | linoleoyl ethanolamide | 0.61 | 0.96 | 0.81 | 0.94 | 0.58 |
| Diacylglycerol | linoleoyl-arachidonoyl-glycerol (18:2/20:4) [1]* | 1.27 | 0.89 | 0.98 | 1.09 | 1.22 |
| Diacylglycerol | linoleoyl-arachidonoyl-glycerol (18:2/20:4) [2]* | 1.30 | 0.91 | 0.91 | 1.54 | 1.39 |
| Diacylglycerol | linoleoyl-docosahexaenoyl-glycerol (18:2/22:6) | 0.88 | 0.41 | 1.00 | 0.59 | 0.89 |
| | [1]* | | | | | |
| Diacylglycerol | linoleoyl-docosahexaenoyl-glycerol (18:2/22:6) | 1.70 | 1.70 | 0.96 | 2.10 | 1.46 |
| | [2]* | | | | | |
| Diacylglycerol | linoleoyl-linolenoyl-glycerol (18:2/18:3) [2]* | 1.02 | 1.45 | 0.74 | 1.67 | 1.32 |
| Diacylglycerol | linoleoyl-linoleoyl-glycerol (18:2/18:2) [1]* | 0.60 | 0.28 | 0.82 | 0.39 | 0.74 |
| Diacylglycerol | linoleoyl-linoleoyl-glycerol (18:2/18:2) [2]* | 1.14 | 1.36 | 0.83 | 1.50 | 1.07 |
| Fatty Acid Metabolism (Acyl Carnitine, | linoleoylcarnitine (C18:2)* | 1.21 | 1.06 | 1.64 | 1.57 | 0.90 |
| Polyunsaturated) | | | | | | |
| Fatty Acid Metabolism (Acyl Choline) | linoleoylcholine* | 0.75 | 0.84 | 0.72 | 0.64 | 0.89 |
| Lysine Metabolism | lysine | 1.00 | 0.99 | 1.02 | 1.14 | 0.99 |
| Pentose Metabolism | lyxonate | 0.89 | 0.97 | 0.98 | 1.00 | 0.67 |
| TCA Cycle | malate | 1.03 | 1.05 | 1.00 | 1.10 | 1.05 |
| Fatty Acid, Dicarboxylate | maleate | 0.78 | 0.90 | 1.05 | 1.02 | 0.89 |
| Fatty Acid Synthesis | malonate | 0.94 | 1.09 | 1.03 | 1.24 | 1.39 |
| Fatty Acid Synthesis | malonylcarnitine | 1.12 | 1.62 | 1.32 | 1.70 | 0.82 |
| Food Component/Plant | maltol sulfate | 1.11 | 0.80 | 0.90 | 0.99 | 1.45 |
| Glycogen Metabolism | maltose | 1.24 | 0.63 | 0.68 | 0.80 | 0.80 |
| Fructose, Mannose and Galactose Metabolism | mannitol/sorbitol | 0.93 | 0.81 | 0.86 | 0.98 | 1.39 |
| Food Component/Plant | mannonate* | 1.07 | 0.77 | 0.79 | 0.75 | 0.73 |
| Fructose, Mannose and Galactose Metabolism | mannose | 1.00 | 0.86 | 0.87 | 0.81 | 0.98 |
| Long Chain Saturated Fatty Acid | margarate (17:0) | 0.73 | 1.16 | 0.95 | 1.07 | 0.54 |
| Fatty Acid Metabolism (Acyl Carnitine, Long | margaroylcarnitine (C17)* | 1.22 | 1.11 | 1.38 | 1.22 | 1.38 |
| Chain Saturated) | | | | | | |
| Methionine, Cysteine, SAM and Taurine | methionine | 0.87 | 1.10 | 1.14 | 0.93 | 0.95 |
| Metabolism | | | | | | |
| Methionine, Cysteine, SAM and Taurine | methionine sulfone | 0.80 | 1.18 | 1.02 | 0.90 | 0.80 |
| Metabolism | | | | | | |
| Methionine, Cysteine, SAM and Taurine | methionine sulfoxide | 0.86 | 1.18 | 1.14 | 1.09 | 1.00 |
| Metabolism | | | | | | |
| Food Component/Plant | methyl glucopyranoside (alpha + beta) | 1.18 | 1.81 | 1.36 | 1.47 | 0.49 |
| Food Component/Plant | methyl indole-3-acetate | 0.94 | 1.57 | 1.10 | 1.15 | 0.46 |
| Food Component/Plant | methyl vanillate sulfate | 0.34 | 1.01 | 0.68 | 1.27 | 0.31 |
| Benzoate Metabolism | methyl-4-hydroxybenzoate sulfate | 0.38 | 1.30 | 0.61 | 0.81 | 0.55 |
| Fatty Acid Metabolism (also BCAA | methylmalonate (MMA) | 0.83 | 0.85 | 0.89 | 0.83 | 1.07 |
| Metabolism) | | | | | | |
| Purine and Pyrimidine Metabolism | methylphosphate | 1.00 | 1.06 | 1.01 | 1.26 | 1.11 |
| Leucine, Isoleucine and Valine Metabolism | methylsuccinate | 1.42 | 0.58 | 0.72 | 0.71 | 3.60 |
| Mevalonate Metabolism | mevalonate | 1.12 | 1.16 | 1.20 | 1.12 | 1.19 |
| Inositol Metabolism | myo-inositol | 1.21 | 1.53 | 1.28 | 1.17 | 1.08 |
| Long Chain Saturated Fatty Acid | myristate (14:0) | 0.79 | 0.94 | 1.46 | 1.01 | 0.49 |
| Fatty Acid Metabolism (Acyl Carnitine, | myristoleoylcarnitine (C14:1)* | 1.31 | 1.18 | 2.41 | 1.51 | 0.97 |
| Monounsaturated) | | | | | | |
| Fatty Acid Metabolism (Acyl Carnitine, Long | myristoylcarnitine (C14) | 1.15 | 1.09 | 1.63 | 1.31 | 0.92 |
| Chain Saturated) | | | | | | |
| Food Component/Plant | N-(2-furoyl)glycine | 1.09 | 0.76 | 0.92 | 1.19 | 1.37 |
| Histidine Metabolism | N-acetyl-1-methylhistidine* | 1.08 | 1.38 | 1.17 | 1.18 | 0.42 |
| Fatty Acid, Amino | N-acetyl-2-aminooctanoate* | 1.05 | 1.12 | 1.30 | 1.22 | 0.93 |
| Histidine Metabolism | N-acetyl-3-methylhistidine* | 1.10 | 1.34 | 0.94 | 1.07 | 0.54 |
| Glutamate Metabolism | N-acetyl-aspartyl-glutamate (NAAG) | 1.18 | 1.28 | 1.27 | 1.08 | 1.06 |
| Pyrimidine Metabolism, Uracil containing | N-acetyl-beta-alanine | 0.94 | 1.27 | 1.15 | 1.32 | 0.77 |
| Polyamine Metabolism | N-acetyl-isoputreanine | 0.81 | 1.01 | 0.99 | 0.82 | 0.16 |
| Alanine and Aspartate Metabolism | N-acetylalanine | 0.96 | 0.87 | 0.97 | 0.93 | 1.34 |
| Urea cycle; Arginine and Proline Metabolism | N-acetylarginine | 0.70 | 0.89 | 1.02 | 1.03 | 1.00 |
| Alanine and Aspartate Metabolism | N-acetylasparagine | 0.79 | 0.61 | 0.85 | 0.82 | 1.40 |
| Alanine and Aspartate Metabolism | N-acetylaspartate (NAA) | 1.27 | 1.16 | 1.24 | 1.14 | 1.09 |
| Urea cycle; Arginine and Proline Metabolism | N-acetylcitrulline | 0.75 | 0.95 | 0.92 | 0.98 | 0.76 |
| Aminosugar Metabolism | N-acetylglucosamine/N-acetylgalactosamine | 1.01 | 0.95 | 1.00 | 1.06 | 0.90 |
| Aminosugar Metabolism | N-acetylglucosaminylasparagine | 1.06 | 1.04 | 0.94 | 1.21 | 0.78 |
| Glutamate Metabolism | N-acetylglutamate | 0.98 | 0.77 | 1.05 | 0.93 | 2.05 |
| Glutamate Metabolism | N-acetylglutamine | 0.99 | 1.10 | 1.03 | 1.25 | 2.08 |
| Glycine, Serine and Threonine Metabolism | N-acetylglycine | 0.84 | 0.72 | 0.74 | 0.89 | 1.24 |
| Histidine Metabolism | N-acetylhistamine | 0.97 | 0.92 | 0.89 | 0.81 | 0.97 |
| Histidine Metabolism | N-acetylhistidine | 0.91 | 0.71 | 0.83 | 0.72 | 1.16 |
| Urea cycle; Arginine and Proline Metabolism | N-acetylhomocitrulline | 0.97 | 0.95 | 1.00 | 0.83 | 0.68 |
| Leucine, Isoleucine and Valine Metabolism | N-acetylisoleucine | 0.68 | 0.69 | 0.76 | 0.88 | 0.86 |
| Tryptophan Metabolism | N-acetylkynurenine (2) | 0.65 | 1.11 | 1.32 | 0.82 | 1.29 |
| Leucine, Isoleucine and Valine Metabolism | N-acetylleucine | 0.70 | 0.70 | 0.78 | 0.86 | 1.23 |
| Methionine, Cysteine, SAM and Taurine | N-acetylmethionine | 0.81 | 0.85 | 0.91 | 0.84 | 1.63 |
| Metabolism | | | | | | |
| Methionine, Cysteine, SAM and Taurine | N-acetylmethionine sulfoxide | 0.92 | 1.14 | 1.12 | 0.97 | 1.38 |
| Metabolism | | | | | | |
| Aminosugar Metabolism | N-acetylneuraminate | 1.16 | 1.19 | 1.15 | 0.97 | 1.08 |
| Phenylalanine Metabolism | N-acetylphenylalanine | 0.75 | 0.91 | 0.98 | 0.90 | 0.61 |
| Urea cycle; Arginine and Proline Metabolism | N-acetylproline | 0.87 | 0.86 | 0.97 | 1.01 | 1.02 |
| Polyamine Metabolism | N-acetylputrescine | 1.28 | 1.16 | 1.24 | 1.22 | 1.24 |
| Food Component/Plant | N-acetylpyrraline | 0.84 | 1.12 | 1.08 | 0.98 | 1.16 |
| Glycine, Serine and Threonine Metabolism | N-acetylserine | 0.96 | 0.82 | 0.92 | 0.89 | 1.85 |
| Methionine, Cysteine, SAM and Taurine | N-acetyltaurine | 0.93 | 1.05 | 1.18 | 1.02 | 2.57 |
| Metabolism | | | | | | |
| Glycine, Serine and Threonine Metabolism | N-acetylthreonine | 0.81 | 0.74 | 0.92 | 0.85 | 1.22 |
| Tryptophan Metabolism | N-acetyltryptophan | 0.64 | 0.82 | 0.86 | 0.91 | 0.79 |
| Tyrosine Metabolism | N-acetyltyrosine | 0.78 | 0.83 | 0.99 | 0.84 | 0.49 |
| Leucine, Isoleucine and Valine Metabolism | N-acetylvaline | 0.74 | 0.79 | 0.87 | 0.95 | 1.19 |
| Ceramides | N-behenoyl-sphingadienine (d18:2/22:0)* | 0.82 | 0.60 | 0.59 | 1.13 | 1.25 |
| Pyrimidine Metabolism, Orotate containing | N-carbamoylaspartate | 1.02 | 1.06 | 0.99 | 1.14 | 1.29 |
| Urea cycle; Arginine and Proline Metabolism | N-delta-acetylornithine | 1.03 | 1.38 | 1.15 | 1.20 | 1.39 |
| Tryptophan Metabolism | N-formylanthranilic acid | 0.79 | 1.20 | 1.05 | 0.98 | 2.53 |
| Methionine, Cysteine, SAM and Taurine | N-formylmethionine | 0.78 | 0.81 | 0.67 | 0.99 | 1.18 |
| Metabolism | | | | | | |
| Tyrosine Metabolism | N-formylphenylalanine | 0.88 | 1.04 | 1.20 | 1.53 | 1.22 |
| Aminosugar Metabolism | N-glycolylneuraminate | 1.14 | 1.25 | 1.25 | 1.34 | 1.39 |
| Endocannabinoid | N-linoleoyltaurine* | 0.89 | 1.20 | 1.18 | 1.47 | 0.79 |
| Glutamate Metabolism | N-methyl-GABA | 0.75 | 0.64 | 0.65 | 0.90 | 0.57 |
| Bacterial/Fungal | N-methylpipecolate | 0.92 | 0.97 | 0.99 | 0.83 | 1.16 |
| Urea cycle; Arginine and Proline Metabolism | N-methylproline | 0.90 | 1.10 | 1.10 | 1.00 | 1.61 |
| Urea cycle; Arginine and Proline Metabolism | N-monomethylarginine | 0.87 | 0.91 | 0.86 | 1.11 | 0.85 |
| Fatty Acid Metabolism (Acyl Glycine) | N-octanoylglycine | 0.90 | 0.75 | 0.75 | 1.86 | 3.71 |
| Endocannabinoid | N-oleoyltaurine | 0.79 | 1.14 | 1.27 | 1.46 | 0.70 |
| Dihydroceramides | N-palmitoyl-phytosphingosine (t18:0/16:0) | 0.74 | 0.80 | 0.76 | 0.81 | 1.04 |
| Dihydroceramides | N-palmitoyl-sphinganine (d18:0/16:0) | 0.65 | 0.71 | 0.69 | 0.92 | 1.39 |
| Ceramides | N-palmitoyl-sphingosine (d18:1/16:0) | 0.87 | 0.77 | 0.77 | 0.74 | 1.12 |
| Fatty Acid Metabolism (Acyl Glycine) | N-palmitoylglycine | 0.61 | 0.97 | 0.88 | 0.85 | 0.71 |
| Endocannabinoid | N-palmitoyltaurine | 0.64 | 1.18 | 1.42 | 1.42 | 0.77 |
| Ceramides | N-stearoyl-sphingosine (d18:1/18:0)* | 1.26 | 0.74 | 0.78 | 0.69 | 1.49 |
| Endocannabinoid | N-stearoylserine* | 0.62 | 1.01 | 0.87 | 1.11 | 0.52 |
| Endocannabinoid | N-stearoyltaurine | 0.75 | 1.23 | 1.08 | 1.29 | 0.60 |
| Modified Peptides | N,N-dimethyl-pro-pro | 1.10 | 1.24 | 1.35 | 1.36 | 0.76 |
| Alanine and Aspartate Metabolism | N,N-dimethylalanine | 0.94 | 1.18 | 1.10 | 0.96 | 3.02 |
| Lysine Metabolism | N,N,N-trimethyl-5-aminovalerate | 1.04 | 1.32 | 1.17 | 1.00 | 5.89 |
| Urea cycle; Arginine and Proline Metabolism | N,N,N-trimethyl-alanylproline betaine (TMAP) | 1.03 | 1.19 | 1.39 | 1.25 | 0.70 |
| Nicotinate and Nicotinamide Metabolism | N1-Methyl-2-pyridone-5-carboxamide | 0.91 | 0.92 | 0.87 | 0.88 | 0.84 |
| Nicotinate and Nicotinamide Metabolism | N1-Methyl-4-pyridone-3-carboxamide | 1.17 | 1.35 | 0.89 | 1.33 | 0.89 |
| Purine Metabolism, Adenine containing | N1-methyladenosine | 1.03 | 0.99 | 1.04 | 0.86 | 0.86 |
| Purine Metabolism, (Hypo)Xanthine/Inosine | N1-methylinosine | 1.12 | 1.03 | 1.07 | 0.97 | 1.03 |
| containing | | | | | | |
| Lysine Metabolism | N2-acetyl,N6,N6-dimethyllysine | 0.89 | 0.99 | 0.89 | 0.91 | 0.82 |
| Lysine Metabolism | N2-acetyllysine | 0.66 | 0.74 | 0.86 | 0.60 | 0.59 |
| Purine Metabolism, Guanine containing | N2,N2-dimethylguanosine | 1.14 | 1.03 | 0.84 | 1.37 | 0.63 |
| Urea cycle; Arginine and Proline Metabolism | N2,N5-diacetylornithine | 0.95 | 1.07 | 0.93 | 1.27 | 0.93 |
| Lysine Metabolism | N2,N6-diacetyllysine | 0.77 | 0.94 | 0.93 | 1.00 | 0.64 |
| Pyrimidine Metabolism, Cytidine containing | N4-acetylcytidine | 1.38 | 2.45 | 1.86 | 0.89 | 1.35 |
| Lysine Metabolism | N6-acetyllysine | 1.00 | 1.14 | 1.18 | 0.93 | 1.12 |
| Purine Metabolism, Adenine containing | N6-carbamoylthreonyladenosine | 0.91 | 0.92 | 1.07 | 0.92 | 0.88 |
| Lysine Metabolism | N6-carboxyethyllysine | 0.96 | 1.07 | 1.03 | 0.96 | 0.65 |
| Advanced Glycation End-product | N6-carboxymethyllysine | 1.13 | 0.69 | 1.16 | 0.92 | 0.50 |
| Lysine Metabolism | N6-methyllysine | 0.92 | 1.04 | 0.93 | 0.88 | 1.21 |
| Purine Metabolism, Adenine containing | N6-succinyladenosine | 1.12 | 1.43 | 1.30 | 1.49 | 0.48 |
| Lysine Metabolism | N6,N6-dimethyllysine | 1.04 | 1.12 | 1.03 | 0.91 | 1.53 |
| Lysine Metabolism | N6,N6,N6-trimethyllysine | 1.10 | 1.15 | 1.08 | 1.30 | 0.97 |
| Chemical | neopentyl glycol adipate** | 1.35 | 1.55 | 2.64 | 1.82 | 0.98 |
| Chemical | neopentyl glycol azelate** | 0.84 | 1.19 | 2.18 | 0.88 | 1.14 |
| Chemical | neopentyl glycol glutarate** | 1.14 | 1.22 | 2.12 | 1.72 | 0.94 |
| Chemical | neopentyl glycol sebacate** | 0.94 | 1.43 | 2.44 | 1.19 | 1.27 |
| Chemical | neopentyl glycol suberate** | 1.00 | 1.43 | 2.64 | 1.35 | 1.14 |
| Chemical | neopentyl glycol undecanedioate** | 0.67 | 1.43 | 2.50 | 1.16 | 1.55 |
| Fatty Acid Metabolism (Acyl Carnitine, | nervonoylcarnitine (C24:1)* | 1.45 | 1.34 | 1.41 | 1.35 | 1.10 |
| Monounsaturated) | | | | | | |
| Nicotinate and Nicotinamide Metabolism | nicotinamide | 0.94 | 1.08 | 0.87 | 1.24 | 0.97 |
| Nicotinate and Nicotinamide Metabolism | nicotinamide N-oxide | 0.94 | 1.13 | 0.97 | 1.20 | 0.91 |
| Nicotinate and Nicotinamide Metabolism | nicotinamide ribonucleotide (NMN) | 1.30 | 0.98 | 0.92 | 1.82 | 0.57 |
| Nicotinate and Nicotinamide Metabolism | nicotinamide riboside | 1.21 | 1.20 | 1.13 | 1.34 | 0.65 |
| Nicotinate and Nicotinamide Metabolism | nicotinate ribonucleoside | 1.02 | 1.60 | 1.03 | 1.17 | 0.80 |
| Long Chain Polyunsaturated Fatty Acid (n3 | nisinate (24:6n3) | 0.92 | 1.62 | 0.94 | 1.34 | 0.36 |
| and n6) | | | | | | |
| Fatty Acid, Dicarboxylate | nonadecanedioate (C19-DC) | 0.97 | 0.99 | 1.75 | 2.11 | 0.97 |
| Long Chain Saturated Fatty Acid | nonadecanoate (19:0) | 0.70 | 1.14 | 0.85 | 1.09 | 0.41 |
| Fatty Acid Metabolism (Acyl Carnitine, | nonanoylcarnitine (C9) | 1.28 | 1.22 | 1.46 | 1.05 | 7.20 |
| Medium Chain) | | | | | | |
| Benzoate Metabolism | o-cresol sulfate | 0.80 | 0.69 | 1.03 | 0.64 | 1.74 |
| Chemical | O-sulfo-L-tyrosine | 0.92 | 0.97 | 1.01 | 1.07 | 0.81 |
| Fatty Acid, Dicarboxylate | octadecadienedioate (C18:2-DC)* | 1.05 | 1.00 | 1.53 | 1.58 | 1.28 |
| Fatty Acid, Dicarboxylate | octadecanedioate (C18-DC) | 0.82 | 0.67 | 1.10 | 1.34 | 0.96 |
| Fatty Acid, Dicarboxylate | octadecenedioate (C18:1-DC) | 0.98 | 0.98 | 1.57 | 1.40 | 1.13 |
| Fatty Acid Metabolism (Acyl Carnitine, | octanoylcarnitine (C8) | 1.38 | 1.21 | 1.45 | 1.10 | 13.88 |
| Medium Chain) | | | | | | |
| Long Chain Monounsaturated Fatty Acid | oleate/vaccenate (18:1) | 0.80 | 1.22 | 1.21 | 1.14 | 0.47 |
| Endocannabinoid | oleoyl ethanolamide | 0.89 | 0.84 | 1.06 | 0.81 | 0.73 |
| Diacylglycerol | oleoyl-arachidonoyl-glycerol (18:1/20:4) [2]* | 1.20 | 0.94 | 0.75 | 1.28 | 1.49 |
| Diacylglycerol | oleoyl-linoleoyl-glycerol (18:1/18:2) [1] | 0.93 | 0.87 | 0.80 | 0.84 | 0.94 |
| Diacylglycerol | oleoyl-linoleoyl-glycerol (18:1/18:2) [2] | 0.97 | 0.85 | 0.82 | 0.98 | 0.92 |
| Fatty Acid Metabolism (Acyl Carnitine, | oleoylcarnitine (C18:1) | 1.24 | 1.09 | 1.62 | 1.42 | 0.93 |
| Monounsaturated) | | | | | | |
| Fatty Acid Metabolism (Acyl Choline) | oleoylcholine | 0.87 | 0.90 | 0.83 | 0.63 | 1.40 |
| Glutathione Metabolism | ophthalmate | 1.79 | 1.13 | 1.95 | 0.88 | 4.39 |
| Urea cycle; Arginine and Proline Metabolism | ornithine | 0.88 | 0.97 | 0.99 | 1.13 | 1.02 |
| Pyrimidine Metabolism, Orotate containing | orotate | 0.95 | 0.96 | 0.88 | 1.27 | 1.10 |
| Pyrimidine Metabolism, Orotate containing | orotidine | 1.09 | 1.00 | 0.98 | 1.17 | 1.08 |
| Ascorbate and Aldarate Metabolism | oxalate (ethanedioate) | 0.99 | 1.01 | 1.08 | 1.01 | 1.32 |
| Tryptophan Metabolism | oxindolylalanine | 0.99 | 0.95 | 1.32 | 0.94 | 1.43 |
| Tyrosine Metabolism | p-cresol glucuronide* | 142.38 | 0.88 | 115.91 | 0.75 | 72.80 |
| Benzoate Metabolism | p-cresol sulfate | 53.54 | 2.52 | 75.15 | 0.62 | 56.31 |
| Benzoate Metabolism | p-hydroxybenzaldehyde | 0.85 | 1.24 | 0.96 | 0.84 | 0.84 |
| Long Chain Saturated Fatty Acid | palmitate (16:0) | 0.83 | 1.11 | 1.03 | 1.15 | 0.67 |
| Long Chain Monounsaturated Fatty Acid | palmitoleate (16:1n7) | 0.79 | 1.06 | 1.84 | 1.13 | 0.50 |
| Diacylglycerol | palmitoleoyl-linoleoyl-glycerol (16:1/18:2) [1]* | 0.73 | 0.61 | 0.88 | 0.55 | 0.72 |
| Fatty Acid Metabolism (Acyl Carnitine, | palmitoleoylcarnitine (C16:1)* | 1.30 | 1.07 | 2.11 | 1.65 | 0.92 |
| Monounsaturated) | | | | | | |
| Dihydrosphingomyelins | palmitoyl dihydrosphingomyelin (d18:0/16:0)* | 0.87 | 0.88 | 0.92 | 0.94 | 1.04 |
| Endocannabinoid | palmitoyl ethanolamide | 0.93 | 0.95 | 1.00 | 1.04 | 0.97 |
| Sphingomyelins | palmitoyl sphingomyelin (d18:1/16:0) | 1.00 | 1.08 | 0.96 | 1.09 | 1.02 |
| Diacylglycerol | palmitoyl-docosahexaenoyl-glycerol (16:0/22:6) | 0.80 | 0.90 | 1.00 | 1.08 | 0.94 |
| | [1]* | | | | | |
| Diacylglycerol | palmitoyl-linoleoyl-glycerol (16:0/18:2) [1]* | 1.07 | 0.87 | 0.84 | 0.90 | 0.77 |
| Diacylglycerol | palmitoyl-linoleoyl-glycerol (16:0/18:2) [2]* | 0.92 | 0.81 | 0.71 | 1.14 | 0.89 |
| Ceramide PEs | palmitoyl-sphingosine-phosphoethanolamine | 0.75 | 0.63 | 0.94 | 0.56 | 0.48 |
| | (d18:1/16:0) | | | | | |
| Fatty Acid Metabolism (Acyl Carnitine, Long | palmitoylcarnitine (C16) | 1.12 | 1.07 | 1.26 | 1.22 | 0.98 |
| Chain Saturated) | | | | | | |
| Fatty Acid Metabolism (Acyl Choline) | palmitoylcholine | 0.94 | 0.92 | 0.80 | 0.70 | 1.20 |
| Pantothenate and CoA Metabolism | pantothenate | 1.03 | 1.25 | 1.26 | 0.87 | 0.97 |
| Medium Chain Fatty Acid | pelargonate (9:0) | 1.21 | 1.18 | 2.01 | 1.28 | 0.99 |
| Long Chain Saturated Fatty Acid | pentadecanoate (15:0) | 0.85 | 1.15 | 1.14 | 1.22 | 0.70 |
| Fatty Acid Metabolism (Acyl Carnitine, Long | pentadecanoylcarnitine (C15)* | 1.11 | 1.13 | 1.62 | 1.51 | 1.30 |
| Chain Saturated) | | | | | | |
| Partially Characterized Molecules | pentose acid* | 0.78 | 0.87 | 0.82 | 0.94 | 0.76 |
| Chemical | perfluorohexanesulfonate (PFHxS) | 0.89 | 1.06 | 1.07 | 1.03 | 0.69 |
| Chemical | perfluorooctanesulfonate (PFOS) | 0.78 | 0.77 | 0.96 | 1.14 | 0.84 |
| Tyrosine Metabolism | phenol glucuronide | 0.86 | 0.86 | 0.86 | 0.86 | 37.21 |
| Tyrosine Metabolism | phenol sulfate | 0.54 | 0.54 | 0.70 | 0.76 | 73.48 |
| Phenylalanine Metabolism | phenylacetate | 2.16 | 1.99 | 2.98 | 0.64 | 4.25 |
| Acetylated Peptides | phenylacetylcarnitine | 8.86 | 3.82 | 11.61 | 0.87 | 5.99 |
| Acetylated Peptides | phenylacetylglutamine | 2.64 | 3.91 | 1.99 | 0.97 | 2.74 |
| Acetylated Peptides | phenylacetylglycine | 1.98 | 2.90 | 1.30 | 0.98 | 2.05 |
| Acetylated Peptides | phenylacetyltaurine | 2.91 | 1.87 | 1.58 | 0.53 | 3.01 |
| Phenylalanine Metabolism | phenylalanine | 0.91 | 1.03 | 0.98 | 1.03 | 0.87 |
| Dipeptide | phenylalanylhydroxyproline* | 0.84 | 0.70 | 0.56 | 0.60 | 0.47 |
| Phenylalanine Metabolism | phenyllactate (PLA) | 0.71 | 0.78 | 0.89 | 0.80 | 1.40 |
| Benzoate Metabolism | phenylpropionylglycine | 1.52 | 0.55 | 0.54 | 8.20 | 65.21 |
| Phenylalanine Metabolism | phenylpyruvate | 0.76 | 1.25 | 1.13 | 0.98 | 1.06 |
| Oxidative Phosphorylation | phosphate | 1.08 | 1.07 | 1.15 | 1.71 | 1.17 |
| Glycolysis, Gluconeogenesis, and Pyruvate | phosphoenolpyruvate (PEP) | 1.45 | 1.27 | 1.60 | 2.90 | 0.36 |
| Metabolism | | | | | | |
| Phospholipid Metabolism | phosphoethanolamine | 1.08 | 1.22 | 1.34 | 1.35 | 0.99 |
| Tryptophan Metabolism | picolinate | 0.68 | 1.07 | 1.18 | 0.71 | 0.69 |
| Fatty Acid Metabolism (Acyl Glycine) | picolinoylglycine | 0.85 | 0.98 | 1.02 | 1.13 | 0.68 |
| Fatty Acid, Dicarboxylate | pimelate (C7-DC) | 1.04 | 1.01 | 1.67 | 1.13 | 0.64 |
| Fatty Acid Metabolism (Acyl Carnitine, | pimeloylcarnitine/3-methyladipoylcarnitine (C7- | 1.36 | 1.32 | 1.26 | 1.22 | 0.58 |
| Dicarboxylate) | DC) | | | | | |
| Lysine Metabolism | pipecolate | 1.03 | 1.27 | 1.00 | 1.36 | 1.65 |
| Urea cycle; Arginine and Proline Metabolism | pro-hydroxy-pro | 0.91 | 1.02 | 0.88 | 0.99 | 0.61 |
| Urea cycle; Arginine and Proline Metabolism | proline | 0.93 | 1.03 | 1.05 | 1.08 | 1.08 |
| Dipeptide | prolylglycine | 0.90 | 0.96 | 0.91 | 0.84 | 1.05 |
| Fatty Acid Metabolism (also BCAA | propionylcarnitine (C3) | 0.98 | 0.93 | 1.19 | 0.81 | 1.59 |
| Metabolism) | | | | | | |
| Fatty Acid Metabolism (also BCAA | propionylglycine | 0.61 | 0.57 | 0.59 | 0.94 | 0.97 |
| Metabolism) | | | | | | |
| Chemical | propylene azelate (2)** | 1.23 | 1.61 | 2.72 | 1.40 | 1.12 |
| Chemical | propylene glutarate (1)** | 1.43 | 1.69 | 3.24 | 3.15 | 1.76 |
| Chemical | propylene glutarate (2)** | 1.44 | 1.71 | 3.07 | 3.13 | 1.70 |
| Chemical | propylene suberate (1)** | 1.36 | 1.48 | 3.08 | 2.55 | 1.29 |
| Chemical | propylene suberate (2)** | 1.40 | 1.48 | 3.00 | 2.52 | 1.30 |
| Pyrimidine Metabolism, Uracil containing | pseudouridine | 1.06 | 1.15 | 1.40 | 0.99 | 0.97 |
| Polyamine Metabolism | putrescine | 1.18 | 1.10 | 0.78 | 1.18 | 0.85 |
| Vitamin B6 Metabolism | pyridoxal | 0.79 | 0.95 | 1.00 | 0.71 | 0.89 |
| Vitamin B6 Metabolism | pyridoxamine | 0.75 | 1.09 | 1.13 | 0.98 | 0.94 |
| Vitamin B6 Metabolism | pyridoxate | 0.91 | 0.88 | 0.96 | 0.91 | 0.86 |
| Food Component/Plant | pyrraline | 0.97 | 1.31 | 1.11 | 1.08 | 1.91 |
| Glycolysis, Gluconeogenesis, and Pyruvate | pyruvate | 0.98 | 1.30 | 0.99 | 0.85 | 1.32 |
| Metabolism | | | | | | |
| Food Component/Plant | quinate | 0.81 | 1.02 | 0.93 | 1.00 | 0.68 |
| Nicotinate and Nicotinamide Metabolism | quinolinate | 1.05 | 1.36 | 1.69 | 1.12 | 2.12 |
| Vitamin A Metabolism | retinal | 1.06 | 1.16 | 1.36 | 1.36 | 1.07 |
| Vitamin A Metabolism | retinol (Vitamin A) | 0.91 | 1.00 | 0.89 | 0.90 | 1.45 |
| Pentose Metabolism | ribitol | 0.94 | 0.93 | 0.98 | 1.04 | 0.91 |
| Riboflavin Metabolism | riboflavin (Vitamin B2) | 0.98 | 1.17 | 1.24 | 1.39 | 0.91 |
| Pentose Metabolism | ribonate | 0.84 | 0.86 | 0.86 | 0.83 | 0.76 |
| Pentose Metabolism | ribose | 0.87 | 1.48 | 1.46 | 0.56 | 0.36 |
| Chemical | S-(3-hydroxypropyl)mercapturic acid (HPMA) | 0.88 | 0.99 | 1.01 | 1.10 | 1.13 |
| Glutamate Metabolism | S-1-pyrroline-5-carboxylate | 0.95 | 1.09 | 1.49 | 1.17 | 1.76 |
| Methionine, Cysteine, SAM and Taurine | S-adenosylhomocysteine (SAH) | 0.48 | 0.27 | 0.41 | 0.63 | 0.92 |
| Metabolism | | | | | | |
| Drug - Other | S-carboxymethyl-L-cysteine | 1.05 | 1.29 | 1.07 | 1.27 | 0.83 |
| Methionine, Cysteine, SAM and Taurine | S-methylcysteine | 1.24 | 1.08 | 1.27 | 0.98 | 1.66 |
| Metabolism | | | | | | |
| Methionine, Cysteine, SAM and Taurine | S-methylcysteine sulfoxide | 1.06 | 0.58 | 1.91 | 0.43 | 1.05 |
| Metabolism | | | | | | |
| Glutathione Metabolism | S-methylglutathione | 0.76 | 0.43 | 0.72 | 0.52 | 1.85 |
| Drug - Topical Agents | salicylate | 0.73 | 1.03 | 1.19 | 0.65 | 1.52 |
| Glycine, Serine and Threonine Metabolism | sarcosine | 0.71 | 0.71 | 0.76 | 0.87 | 0.73 |
| Fatty Acid, Dicarboxylate | sebacate (C10-DC) | 1.11 | 1.12 | 1.97 | 1.39 | 0.77 |
| Pentose Metabolism | sedoheptulose | 0.97 | 1.17 | 0.94 | 1.36 | 0.83 |
| Pentose Phosphate Pathway | sedoheptulose-7-phosphate | 1.24 | 1.45 | 1.56 | 2.67 | 0.24 |
| Glycine, Serine and Threonine Metabolism | serine | 1.05 | 0.98 | 1.09 | 1.04 | 0.95 |
| Tryptophan Metabolism | serotonin | 0.91 | 0.82 | 0.95 | 0.72 | 0.31 |
| Polyamine Metabolism | spermidine | 1.24 | 1.06 | 0.98 | 1.20 | 0.62 |
| Polyamine Metabolism | spermine | 1.56 | 1.10 | 1.70 | 1.16 | 0.89 |
| Sphingolipid Synthesis | sphinganine | 1.56 | 1.22 | 1.18 | 1.32 | 1.33 |
| Sphingolipid Synthesis | sphinganine-1-phosphate | 1.03 | 0.95 | 0.94 | 0.84 | 0.71 |
| Sphingomyelins | sphingomyelin (d17:1/14:0, d16:1/15:0)* | 0.84 | 0.94 | 0.89 | 0.86 | 0.77 |
| Sphingomyelins | sphingomyelin (d17:1/16:0, d18:1/15:0, | 0.81 | 0.85 | 1.05 | 0.85 | 0.69 |
| | d16:1/17:0)* | | | | | |
| Sphingomyelins | sphingomyelin (d17:2/16:0, d18:2/15:0)* | 0.93 | 0.89 | 0.75 | 0.86 | 0.89 |
| Dihydrosphingomyelins | sphingomyelin (d18:0/18:0, d19:0/17:0)* | 1.21 | 0.85 | 1.07 | 0.98 | 1.14 |
| Dihydrosphingomyelins | sphingomyelin (d18:0/20:0, d16:0/22:0)* | 0.87 | 0.52 | 0.79 | 0.59 | 1.05 |
| Sphingomyelins | sphingomyelin (d18:1/14:0, d16:1/16:0)* | 0.89 | 0.91 | 0.90 | 0.95 | 0.81 |
| Sphingomyelins | sphingomyelin (d18:1/17:0, d17:1/18:0, | 0.84 | 0.85 | 0.95 | 0.88 | 1.08 |
| | d19:1/16:0) | | | | | |
| Sphingomyelins | sphingomyelin (d18:1/18:1, d18:2/18:0) | 0.97 | 1.01 | 0.98 | 1.17 | 0.90 |
| Sphingomyelins | sphingomyelin (d18:1/19:0, d19:1/18:0)* | 0.92 | 0.77 | 0.95 | 0.89 | 0.91 |
| Sphingomyelins | sphingomyelin (d18:1/20:0, d16:1/22:0)* | 0.92 | 0.73 | 0.90 | 0.92 | 1.02 |
| Sphingomyelins | sphingomyelin (d18:1/20:1, d18:2/20:0)* | 1.04 | 1.01 | 1.03 | 1.24 | 0.89 |
| Sphingomyelins | sphingomyelin (d18:1/21:0, d17:1/22:0, | 0.91 | 0.71 | 0.77 | 1.07 | 0.92 |
| | d16:1/23:0)* | | | | | |
| Sphingomyelins | sphingomyelin (d18:1/22:1, d18:2/22:0, | 0.98 | 0.84 | 0.87 | 1.18 | 0.95 |
| | d16:1/24:1)* | | | | | |
| Sphingomyelins | sphingomyelin (d18:1/22:2, d18:2/22:1, | 1.15 | 1.14 | 1.00 | 1.41 | 0.91 |
| | d16:1/24:2)* | | | | | |
| Sphingomyelins | sphingomyelin (d18:1/24:1, d18:2/24:0)* | 1.35 | 1.55 | 0.91 | 1.52 | 1.29 |
| Sphingomyelins | sphingomyelin (d18:1/25:0, d19:0/24:1, | 1.16 | 1.16 | 1.14 | 1.11 | 0.97 |
| | d20:1/23:0, d19:1/24:0)* | | | | | |
| Sphingomyelins | sphingomyelin (d18:2/14:0, d18:1/14:1)* | 0.89 | 0.88 | 0.86 | 0.90 | 0.84 |
| Sphingomyelins | sphingomyelin (d18:2/16:0, d18:1/16:1)* | 1.00 | 1.01 | 0.91 | 1.12 | 0.99 |
| Sphingomyelins | sphingomyelin (d18:2/18:1)* | 1.13 | 1.04 | 1.11 | 1.01 | 0.84 |
| Sphingomyelins | sphingomyelin (d18:2/21:0, d16:2/23:0)* | 1.06 | 0.87 | 0.93 | 1.21 | 0.81 |
| Sphingomyelins | sphingomyelin (d18:2/23:0, d18:1/23:1, | 0.85 | 0.80 | 0.88 | 0.82 | 0.74 |
| | d17:1/24:1)* | | | | | |
| Sphingomyelins | sphingomyelin (d18:2/23:1)* | 0.97 | 0.97 | 0.99 | 0.98 | 0.77 |
| Sphingomyelins | sphingomyelin (d18:2/24:1, d18:1/24:2)* | 1.01 | 1.04 | 0.99 | 1.01 | 0.91 |
| Sphingomyelins | sphingomyelin (d18:2/24:2)* | 1.05 | 1.16 | 1.04 | 1.18 | 0.93 |
| Sphingosines | sphingosine | 1.27 | 1.12 | 1.12 | 1.21 | 1.19 |
| Sphingosines | sphingosine 1-phosphate | 1.06 | 1.03 | 0.89 | 0.95 | 0.72 |
| Food Component/Plant | stachydrine | 1.07 | 1.25 | 1.21 | 1.01 | 1.26 |
| Long Chain Saturated Fatty Acid | stearate (18:0) | 0.78 | 1.05 | 0.93 | 1.09 | 0.65 |
| Long Chain Polyunsaturated Fatty Acid (n3 | stearidonate (18:4n3) | 0.87 | 0.91 | 1.24 | 1.14 | 0.60 |
| and n6) | | | | | | |
| Endocannabinoid | stearoyl ethanolamide | 0.95 | 0.88 | 0.99 | 1.11 | 0.80 |
| Sphingomyelins | stearoyl sphingomyelin (d18:1/18:0) | 0.99 | 1.02 | 1.03 | 1.18 | 1.04 |
| Fatty Acid Metabolism (Acyl Carnitine, Long | stearoylcarnitine (C18) | 1.41 | 1.18 | 1.21 | 1.52 | 1.23 |
| Chain Saturated) | | | | | | |
| Fatty Acid Metabolism (Acyl Choline) | stearoylcholine* | 0.99 | 0.77 | 0.81 | 0.67 | 1.10 |
| Fatty Acid, Dicarboxylate | suberate (C8-DC) | 1.13 | 1.09 | 1.92 | 1.36 | 0.70 |
| Fatty Acid Metabolism (Acyl Carnitine, | suberoylcarnitine (C8-DC) | 1.58 | 1.10 | 1.97 | 1.30 | 1.55 |
| Dicarboxylate) | | | | | | |
| TCA Cycle | succinate | 0.95 | 0.83 | 0.77 | 0.92 | 0.98 |
| Methionine, Cysteine, SAM and Taurine | succinoyltaurine | 0.73 | 0.42 | 0.68 | 0.59 | 0.75 |
| Metabolism | | | | | | |
| TCA Cycle | succinylcarnitine (C4-DC) | 1.15 | 1.29 | 1.24 | 1.70 | 0.38 |
| Chemical | sulfate* | 0.92 | 0.99 | 0.98 | 1.02 | 0.96 |
| Food Component/Plant | tartarate | 0.84 | 0.96 | 0.87 | 0.69 | 0.68 |
| Food Component/Plant | tartronate (hydroxymalonate) | 0.98 | 1.02 | 0.92 | 0.91 | 0.85 |
| Methionine, Cysteine, SAM and Taurine | taurine | 1.02 | 1.04 | 1.19 | 1.18 | 1.09 |
| Metabolism | | | | | | |
| Primary Bile Acid Metabolism | tauro-beta-muricholate | 0.32 | 0.35 | 6.75 | 0.61 | 0.24 |
| Primary Bile Acid Metabolism | taurochenodeoxycholate | 0.45 | 0.64 | 4.70 | 0.93 | 0.73 |
| Primary Bile Acid Metabolism | taurocholate | 0.33 | 0.47 | 2.23 | 0.61 | 0.45 |
| Methionine, Cysteine, SAM and Taurine | taurocyamine | 1.03 | 0.72 | 1.24 | 0.94 | 1.08 |
| Metabolism | | | | | | |
| Secondary Bile Acid Metabolism | taurodeoxycholate | 1.11 | 3.25 | 0.71 | 1.09 | 367.34 |
| Secondary Bile Acid Metabolism | taurohyodeoxycholic acid | 0.66 | 0.66 | 2.11 | 0.67 | 5.04 |
| Secondary Bile Acid Metabolism | taurolithocholate 3-sulfate | 2.90 | 7.50 | 3.30 | 0.87 | 0.89 |
| Secondary Bile Acid Metabolism | tauroursodeoxycholate | 0.63 | 0.70 | 12.94 | 1.05 | 0.56 |
| Fatty Acid, Dicarboxylate | tetradecadienedioate (C14:2-DC)* | 1.07 | 1.09 | 1.71 | 1.66 | 0.75 |
| Long Chain Polyunsaturated Fatty Acid (n3 | tetradecadienoate (14:2)* | 1.14 | 0.96 | 1.87 | 1.21 | 0.51 |
| and n6) | | | | | | |
| Fatty Acid, Dicarboxylate | tetradecanedioate (C14-DC) | 1.03 | 0.98 | 1.52 | 1.34 | 0.89 |
| Thiamine Metabolism | thiamin (Vitamin B1) | 0.81 | 0.95 | 0.75 | 1.12 | 0.98 |
| Thiamine Metabolism | thiamin monophosphate | 0.72 | 0.77 | 0.67 | 0.77 | 1.02 |
| Chemical | thioproline | 1.10 | 0.97 | 1.12 | 0.99 | 1.10 |
| Ascorbate and Aldarate Metabolism | threonate | 0.98 | 0.99 | 1.06 | 0.96 | 1.48 |
| Glycine, Serine and Threonine Metabolism | threonine | 0.95 | 1.02 | 1.04 | 1.07 | 1.07 |
| Pyrimidine Metabolism, Thymine containing | thymidine | 1.11 | 0.86 | 1.09 | 0.99 | 1.25 |
| Pyrimidine Metabolism, Thymine containing | thymine | 1.11 | 1.66 | 1.24 | 1.80 | 1.23 |
| Tyrosine Metabolism | thyroxine | 0.73 | 1.10 | 1.11 | 0.88 | 0.65 |
| Leucine, Isoleucine and Valine Metabolism | tigloylglycine | 0.92 | 0.74 | 0.65 | 1.30 | 0.78 |
| Leucine, Isoleucine and Valine Metabolism | tiglylcarnitine (C5:1-DC) | 0.95 | 1.06 | 1.37 | 1.09 | 1.06 |
| Fatty Acid Metabolism (Acyl Glycine) | trans-2-hexenoylglycine | 1.13 | 0.76 | 0.67 | 1.48 | 2.66 |
| Fatty Acid Metabolism (Acyl Glycine) | trans-3,4-methyleneheptanoylglycine | 0.79 | 0.68 | 0.88 | 1.28 | 0.92 |
| Urea cycle; Arginine and Proline Metabolism | trans-4-hydroxyproline | 0.99 | 1.11 | 1.06 | 1.22 | 0.94 |
| Histidine Metabolism | trans-urocanate | 0.86 | 1.34 | 1.15 | 1.18 | 0.77 |
| Sphingomyelins | tricosanoyl sphingomyelin (d18:1/23:0)* | 0.75 | 0.53 | 0.92 | 0.54 | 0.84 |
| Fatty Acid, Dicarboxylate | tridecanedioate (C13-DC) | 1.25 | 1.17 | 2.26 | 1.45 | 0.81 |
| Nicotinate and Nicotinamide Metabolism | trigonelline (N′-methylnicotinate) | 0.92 | 1.08 | 1.04 | 1.07 | 1.12 |
| Phospholipid Metabolism | trimethylamine N-oxide | 2.53 | 0.56 | 1.25 | 1.31 | 96.73 |
| Tryptophan Metabolism | tryptophan | 0.84 | 0.96 | 0.93 | 0.94 | 0.83 |
| Tyrosine Metabolism | tyrosine | 0.87 | 0.92 | 0.92 | 0.97 | 0.90 |
| Fatty Acid, Dicarboxylate | undecanedioate (C11-DC) | 1.20 | 1.15 | 2.02 | 1.40 | 0.82 |
| Medium Chain Fatty Acid | undecanoate (11:0) | 1.18 | 1.23 | 1.89 | 1.36 | 0.98 |
| Fatty Acid Metabolism (Acyl Carnitine, | undecenoylcarnitine (C11:1) | 1.09 | 0.80 | 1.81 | 0.87 | 1.10 |
| Monounsaturated) | | | | | | |
| Pyrimidine Metabolism, Uracil containing | uracil | 1.15 | 1.08 | 0.97 | 1.33 | 1.26 |
| Purine Metabolism, (Hypo)Xanthine/Inosine | urate | 1.03 | 0.85 | 0.86 | 0.93 | 0.92 |
| containing | | | | | | |
| Urea cycle; Arginine and Proline Metabolism | urea | 0.83 | 1.04 | 1.07 | 1.07 | 0.97 |
| Pyrimidine Metabolism, Uracil containing | uridine | 0.89 | 0.72 | 0.78 | 0.99 | 0.95 |
| Pyrimidine Metabolism, Uracil containing | uridine 5′-monophosphate (UMP) | 1.21 | 0.43 | 0.96 | 2.60 | 0.58 |
| Secondary Bile Acid Metabolism | ursodeoxycholate | 11.99 | 14.60 | 45.21 | 82.94 | 0.85 |
| Short Chain Fatty Acid | valerate (5:0) | 1.04 | 0.87 | 1.55 | 1.32 | 4.82 |
| Fatty Acid Metabolism (Acyl Carnitine, Short | valerylcarnitine (C5) | 1.21 | 1.08 | 1.39 | 0.76 | 4.00 |
| Chain) | | | | | | |
| Fatty Acid Metabolism (Acyl Glycine) | valerylglycine | 0.55 | 0.37 | 0.43 | 0.84 | 1.61 |
| Leucine, Isoleucine and Valine Metabolism | valine | 0.80 | 0.87 | 0.86 | 0.93 | 0.85 |
| Tyrosine Metabolism | vanillactate | 1.27 | 1.04 | 1.01 | 0.93 | 1.36 |
| Food Component/Plant | vanillic acid glycine | 0.98 | 0.66 | 0.79 | 0.86 | 1.05 |
| Purine Metabolism, (Hypo)Xanthine/Inosine | xanthine | 1.12 | 1.63 | 1.55 | 1.06 | 0.62 |
| containing | | | | | | |
| Purine Metabolism, (Hypo)Xanthine/Inosine | xanthosine | 1.00 | 0.81 | 0.83 | 0.92 | 0.88 |
| containing | | | | | | |
| Tryptophan Metabolism | xanthurenate | 0.64 | 0.66 | 0.79 | 0.94 | 0.84 |

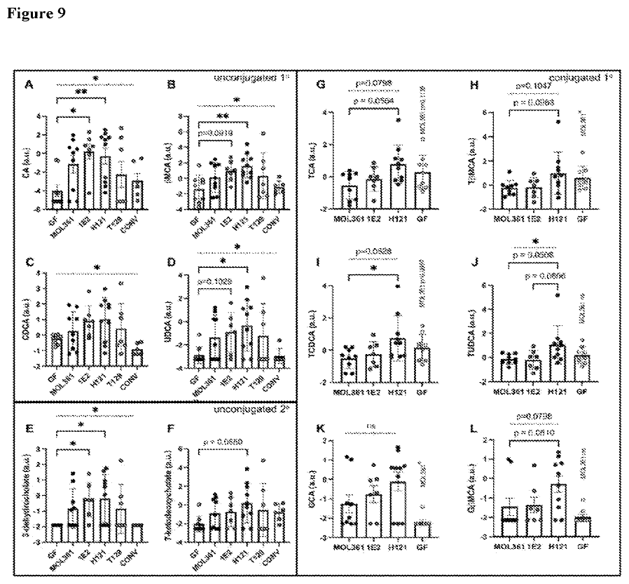

Bile acids can affect circulating host lipids by altering fat digestion and systemic hormonal signaling⁴⁰). They are produced by the host and released into the small intestine, where they promote digestion of fats by facilitating micelle formation, and can also act through receptors like the farnesoid X receptor (FXR)⁴⁰ and GPBARI/TGR⁴¹. Gut bacteria can modify bile acids, primarily through transformations like deconjugation. Previous reports found that MOL361 can broadly modify bile acids in vitro²¹, so to determine if these abilities allowed MOL361 and other Turicibacter strains to modify host bile acids in vivo, the studyprofiled serum bile acids in Turicibacter-monocolonized mice. Though each strain uniquely impacted host serum metabolites, the study noted some patterns in bile acids across monocolonized mice in comparison to either their GF or CONV controls. (Note: bile species with amino acid conjugants are typically referred to as “bile salts,” but for simplicity, the study will herein refer to both conjugated and unconjugated bile species as “bile acids”). Colonization with all of the Turicibacter strains led to a general increase in serum levels of unconjugated primary bile acids like cholate (CA), chenodeoxycholate (CDCA), and β-muricholate (βMCA) (FIG. 9a-d), and a similar rise in unconjugated secondary bile acids 3-dehydrocholate and 7-ketodeoxycholate (FIG. 9e,f). These responses were highly variable in the case of T129 colonization, leading us to de-emphasize this strain for subsequent experiments. In all, these results suggest that these Turicibacter strains are able to impact host bile acids, potentially by deconjugating them in the gut.

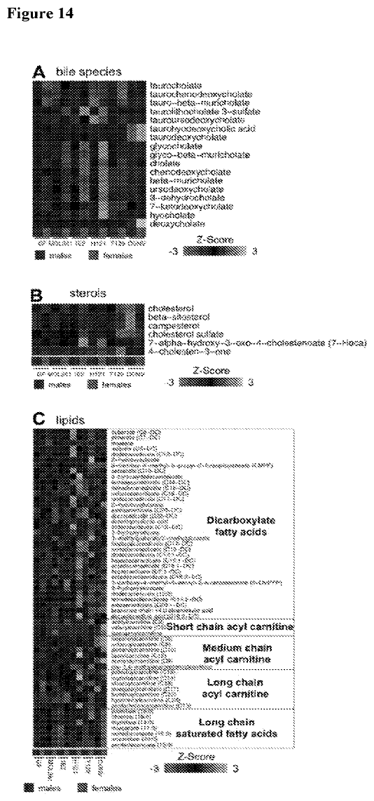

To determine potential explanations for increases in unconjugated bile species, it was observed that the levels of conjugated bile acids differed between animals colonized by different Turicibacter strains, with the clearest delineation separating H121 from MOL361 and 1E2. MOL361- and 1E2-colonized animals generally had lower levels of taurine-conjugated primary bile acids in comparison to H121-colonized animals (FIG. 9g-j), whereas H121 colonization led to an increase in glyco-beta-muricholic acid (GBMCA) (FIG. 9k). Female mice displayed the most significant Turicibacter-associated changes in circulating bile species, lipids, and cholesterol, indicating sex-dependent responses to Turicibacter colonization (FIG. 14a-c). This sex difference has also been reported in C57BL/6 mice monocolonized with T. sanguinis MOL361, albeit with a directionally different change in adipocyte size³⁸. Overall, these data demonstrate that colonization with Turicibacter alters serum bile acids, lipids, and cholesterol, as well as host fat mass. Further, while some changes like increased unconjugated bile acids were conserved across Turicibacter colonizations, changes to specific conjugated bile acids differed between Turicibacter strains, with MOL361 and 1E2 leading to lower levels of taurine-conjugated bile acids than H121 colonization.

Turicibacter Strains Differ in their Ability to Modify Host Bile Acids

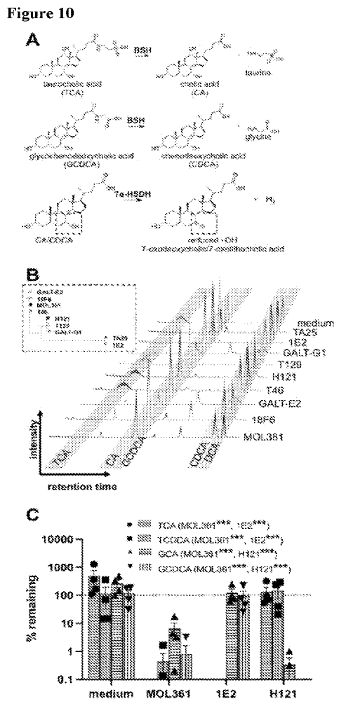

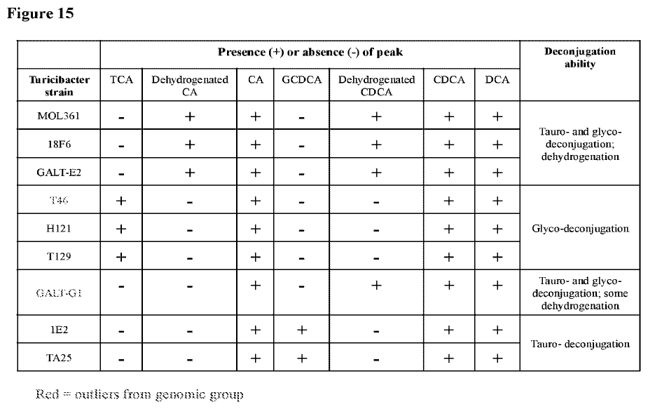

Based on the large genetic variation between the Turicibacter strains (FIG. 7) and the differences in serum lipid and bile acid profiles seen in response to colonization with different strains (FIG. 8), the study posited that these strains differ in their ability to modify bile acids. To test this, the study grew each of our nine isolates to stationary phase in rich medium supplemented with a sub-inhibitory concentration of five bile species²¹: cholic acid, chenodeoxycholic acid deoxycholic acid (DCA), taurocholic acid (TCA) and glycochenodeoxycholic acid (GCDCA). The study then used liquid chromatography-mass spectrometry (LC-MS) to characterize the resulting bile transformations performed by each isolate (FIG. 10a). The study discovered that the strains not only differed in their ability to modify this combination of bile species, but also that these capabilities generally mirrored the groupings identified in genomic comparisons (FIG. 10b). MOL361, 18F6, and GALT-E2 deconjugated both tauro- and glyco-bile acids, and also dehydrogenated CA and CDCA (FIG. 10b). 1E2 and TA25 deconjugated tauro-bile acids, but did not detectably deconjugate glyco-bile acids or perform dehydrogenation (FIG. 10b). H121 and T129 deconjugated glyco-bile acids, but did not readily deconjugate tauro-bile acids nor perform detectable dehydrogenation (FIG. 10b). T46 and GALT-G1 did not have bile-modifying capacity that mirrored their genetic phylogeny; T46 genomically resembled the MOL361 group but performed modifications similar to the H121 group (i.e. glyco but not tauro-deconjugation, minimal dehydrogenation), whereas GALT-G1, which genomically resembled the H121 group, performed more MOL361-like transformations (i.e. glyco- and tauro-deconjugation, dehydrogenation of CDCA) (FIG. 7, 10b, FIG. 15). Overall, each strain performed at least one of three bile transformations, with some showing capacity for all three (FIG. 10b, FIG. 15).

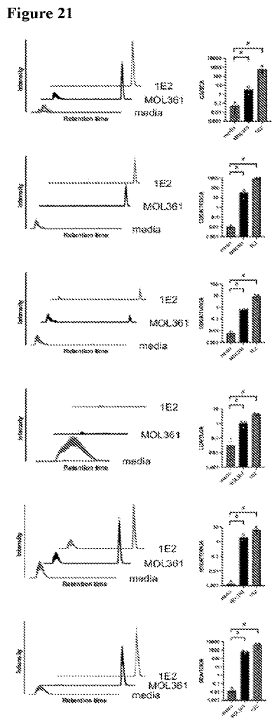

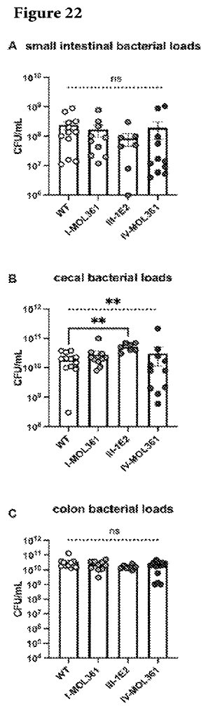

To confirm these intragroup distinctions, the study chose one isolate from each of the subgroups (MOL361, H121, 1E2), and grew them in the presence of four primary conjugated bile acids: TCA, taurochenodeoxycholic acid (TCDCA), glycocholic acid (GCA), and GCDCA. This supported the same pattern seen above; MOL361 deconjugated both groups of bile acids, 1E2 preferentially deconjugated tauro-conjugates, and H121 preferentially deconjugated glyco-conjugates (FIG. 10c). MOL361 and 1E2 displayed broad deconjugation of tauro-conjugates and were able to process at least six taurine-conjugated bile acids (FIG. 21). These data reveal that while all tested strains are efficient modifiers of bile species, their specific transformations differ in a strain-dependent manner, potentially reflecting functions that influence their differential effects on host lipid biology.

Turicibacter Genomes have Different Repertoires of Bile Salt Hydrolases

The different bile modification abilities across strain subgroups suggested that each contained unique repertoires of bile-modifying genes.

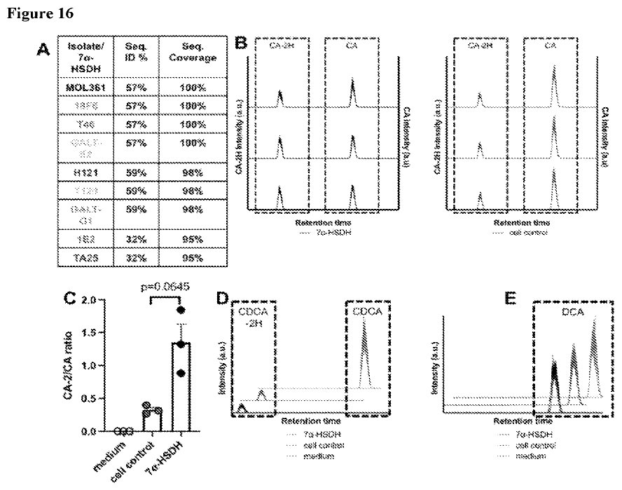

Certain bacteria from the gut microbiota dehydrogenate hydroxyl groups from the steroid core of bile acids⁴², increasing their polarity and modulating their affinity for host bile acid receptors⁴³. The study searched the Turicibacter genomes for homologs of the characterized 7α-hydroxysteroid dehydrogenase (7α-HSDH21) from Clostridium absolum⁴⁴. This revealed genes with 57% amino acid identity in MOL361, 18F6, T46, and GALT-E2, and homologs with 59% amino acid identity in H121 and T129 (FIG. 16a). Though the H121-derived putative homolog had higher overall sequence identity than the MOL361-derived homolog, it lacked certain features predicted to be critical for dehydrogenase activity, such as the analogous Asp38 that is catalytically critical for this reaction⁴⁴. Because in vitro experiments showed only isolates from the MOL361 group performed bile dehydrogenation, the study cloned the putative 7α-HSDH homolog from MOL361 into E. coli C41-pLys and then grew these cells in individual unconjugated bile acids that can be dehydrogenated: CA, CDCA, and DCA. Indeed, the protein encoded by the gene removed the mass equivalent of two hydrogens from CA (FIG. 16b, c). Background transformation by E. coli prevented clear evidence of CDCA dehydrogenation by this putative 7α-HSDH homolog (FIG. 16d), but this homolog did not act on DCA (FIG. 16e), supporting its annotation as a 7α-HSDH.

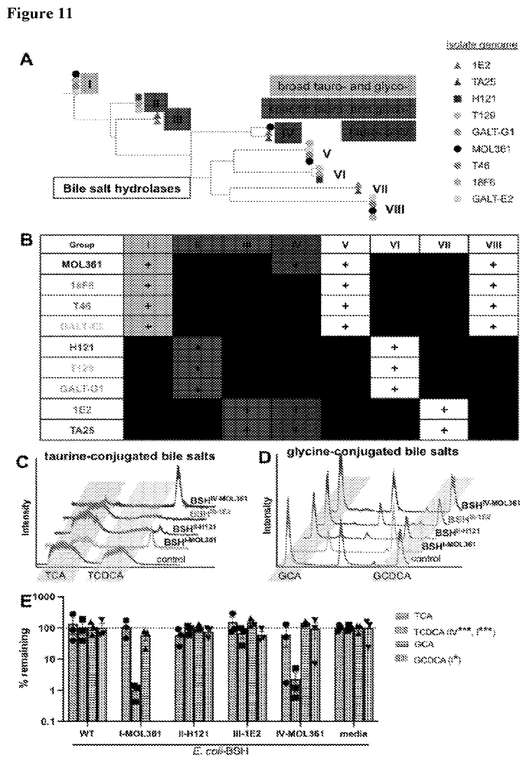

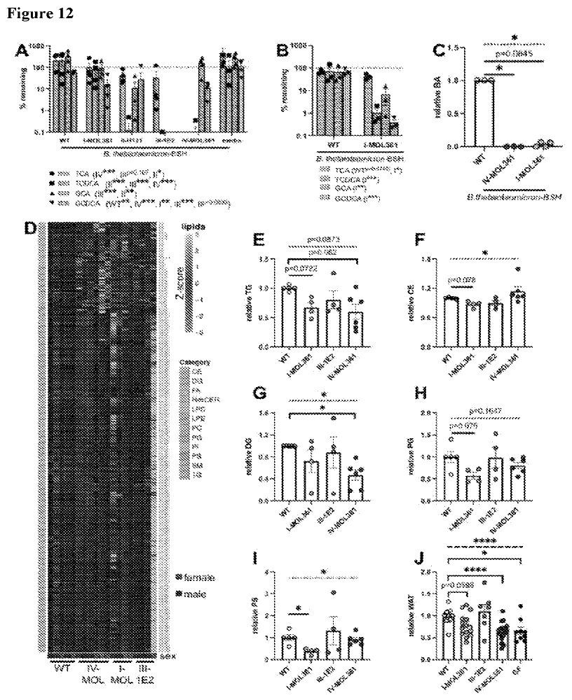

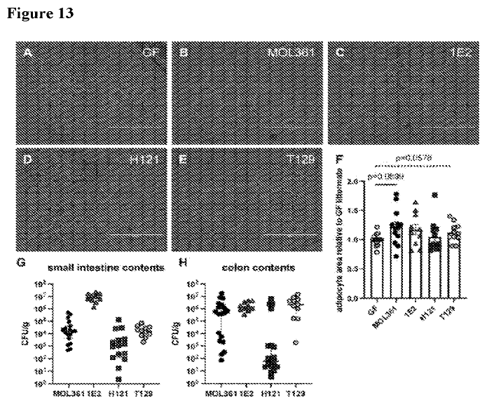

Conjugation increases bile acid solubility and emulsification ability⁴⁵, while deconjugation reverses these effects, leading to decreased dietary lipid absorption. To identify Turicibacter bile salt hydrolase (bsh) genes responsible for the strain-specific differences in bile deconjugation, the study first searched our assembled genomes for annotations of “choloylglycine hydrolase,” the broad category that includes these genes. Of these annotated genes, the study identified eight groups of potentially homologous sequences, and found that each Turicibacter strain encodes putative choloylglycine hydrolases from at least two of the eight groups (FIG. 11a, b). Isolates within the same phylogenetic and phenotypic subgroups largely shared similar sequences (FIG. 11a, b). To assay the function of the strain-specific putative bsh genes, the study cloned and individually expressed one representative sequence from each of the eight putative choloylglycine hydrolase groups in E. coli C41-pLys and measured the ability of these engineered bacteria to perform the deconjugations the study observed in the native Turicibacter. The study cultured the individual E. coli strains to stationary phase in the presence of two tauro-(TCA, TCDCA) or glyco-(GCA, GCDCA) bile acids, then measured their ability to deconjugate these bile acid pools. The study found that E. coli expressing four of the eight putative bsh gene groups showed deconjugation activity against at least one of the bile acids (FIG. 11c-e). From MOL361, one BSH (group IV) is tauro-specific, and one (group I) deconjugates both glyco- and tauro-conjugates (FIG. 11c, d). 1E2 shares a tauro-specific BSH with MOL361 (group IV), and has another BSH (group III) with moderate activity on TCDCA (FIG. 11c, d). H121 has a BSH (group II) with activity on TCDCA and GCDCA (FIG. 11c, d), although this was blunted when presented with the combined four bile acids (FIG. 11e), potentially because of competitive inhibition. Collectively, these findings reveal that Turicibacter strains contain a range of bile salt hydrolases with deconjugation preferences for different bile acids.

Strain- and Substrate-Specific Bile Salt Hydrolases from Turicibacter Differentially Alter Host Lipid Composition

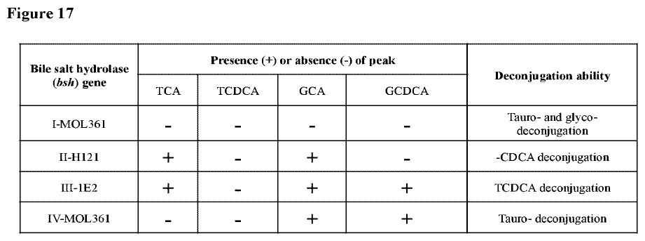

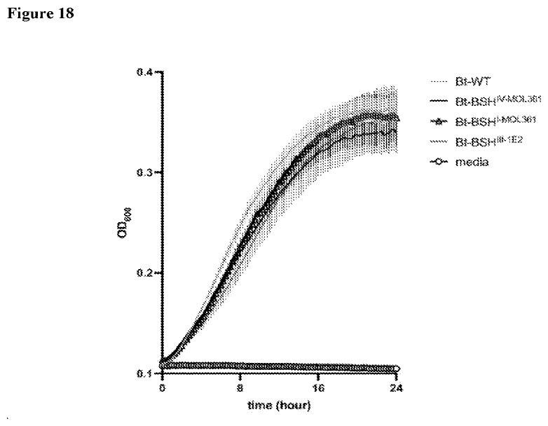

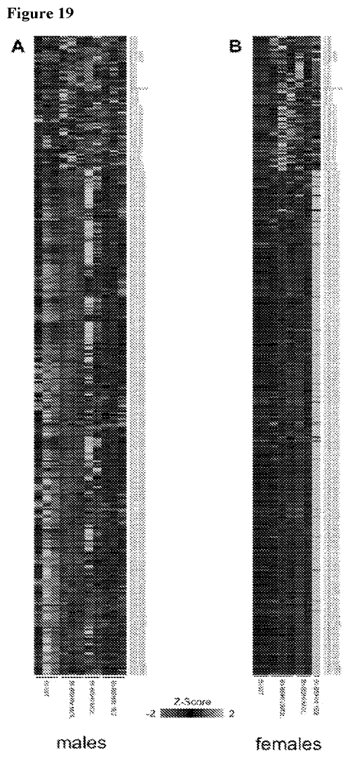

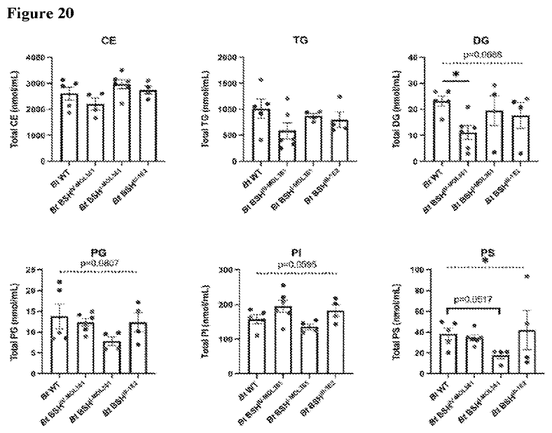

Given that Turicibacter colonization broadly modified host lipid and bile pools (FIG. 8), and that bile transformations have been previously shown to alter host lipids^{46,47}, the study predicted that expressing Turicibacter bile-modifying genes outside the context of Turicibacter colonization would be sufficient to impact host lipid biology. To measure the individual effects of these bile acid transformations, the study expressed Turicibacter bsh genes off of a genomically-integrated high expression vector⁴⁸ in the common gut bacterium Bacteroides thetaiotaomicron. This bacterium was chosen because it stably colonizes the murine gut, and unlike E. coli C41-pLys, contains a homolog of a characterized 7α-HSDH similar to that of T. sanguinis MOL361^{49,50}, allowing the engineered bacteria to more completely mimic Turicibacter bile transformations. These strains generally transformed tauro- and glyco-conjugated bile acids as predicted based on the BSH characterization in E. coli, including preferential TCDCA transformation, indicating they were capable of performing Turicibacter-like bile transformations (FIG. 12a, FIG. 17). However, the B. thetaiotaomicron strain that expressed bsh (group III) from strain 1E2 more completely transformed glyco-conjugated bile acids than the strain expressing bsh (group II) from H121, counter to the E. coli findings. This observation led us to not use the bsh (group II)-expressing strain further. Additionally, although a signficiant in vitro growth defect in the engineered B. thetaiotaomicron strains was not noticed (FIG. 22), we observed a bile transformation delay in B. thetaiotaomicron expressing bsh (group I) from MOL361, that the study could compensate for by extending the growth period (FIG. 12b,).

The study monocolonized mice with the bsh-expressing B. thetaiotaomicron strains and assessed their circulating lipid profiles and abdominal WAT mass. It was found that the engineered strains colonized the gnotobiotic mouse gut at least as well as the parental strain (Supplemental FIG. 8), and that expressing individual Turicibacter bsh genes in the B. thetaiotaomicron background was sufficient to significantly alter host colonic bile levels (FIG. 12c) and the absolute abundance of 346 circulating lipid species (FIG. 12d, Table 7). In particular, expression of either the group I or group IV bsh led to a decrease in triglycerides (FIG. 12d, e). Expressing the tauro-specific bsh (group IV) from MOL361 also decreased diacylglycerides (FIG. 12g). Expressing the broader specificity bsh (group I) from MOL361 also led to a decrease of phosphatidylglycine, phosphatidylserine, and cholesterol (FIG. 12d-f, h, i). Despite having broad capacity for transformation, B. thetaiotaomicron expressing bsh (group III) from 1E2 did not alter host lipid profiles as much as the other strains (FIG. 12d-i). On a tissue-level, bsh-expression also altered WAT storage in the colonized mice, with the broad tauro-deconjugating BSHs (group I and IV) significantly reducing WAT mass (FIG. 12j). Similar to our findings with Turicibacter monocolonization, we also observed a sex difference in BSH responses, with male mice showing more consistent decrease in triglycerides in response to the tauro-specific BSH from MOL361, and females showing more consistent triglyceride responses to the broadly deconjugating BSH (Supplementary FIG. 9).

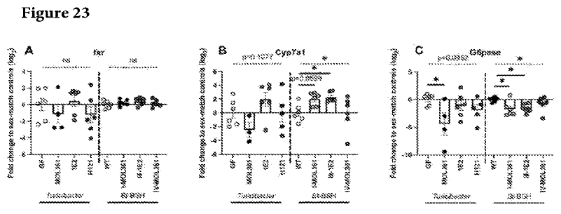

To further explore potential factors that may drive the cholesterol and WAT alterations in response to Turicibacter colonization and bsh expression, liver transcript levels were measured of farnesoid X receptor, (Fxr), a key nuclear receptor for bile acids; cytochrome P450 Family A Subfamily A Member 1/Cholesterol 7a Hydroxylase (Cyp7a1), the rate-limiting enzyme for conversion of cholesterol into bile acids; and glucose-6-phosphatase (G6pase), a key enzyme for gluconeogenesis. There were no differences in Fxr transcript levels across any of the native and engineered bacterial colonization conditions (FIG. 23a). However, similar increases in Cyp7a1 expression (FIG. 23b) and decreases in G6pase expression (FIG. 23c) were found between the Turicibacter and bsh colonizations. In all, these results demonstrate that expressing strain-specific bsh genes from Turicibacter, especially those able to process the abundant taurine-conjugated bile acids present in the murine intestine, is sufficient to drastically alter host cholesterol, bile, and lipid biology.

### Discussion

Results from this study show that Turicibacter bacteria from the mammalian gut microbiota modulate host bile and lipid compositions in a strain-dependent manner. The study identified and characterized five novel Turicibacter genes capable of performing bile transformations (four bsh, one 7α-HSDH), and revealed that expression of individual bsh genes is sufficient to broadly and differentially alter host lipid profiles. Further, the study found that while bile-transforming genes are present in all our surveyed Turicibacter strains, the specific transformation capacity of BSH variants differed by strain in a manner consistent with host environment co-evolution: bile acids in the human gut are a mix of taurine- and glycine-conjugants, whereas murine bile acids are predominantly taurine-conjugants^{51,52}, providing different bile environments that are preferentially processed by Turicibacter strains isolated from their respective gastrointestinal tracts. This close connection between host-specific bile composition and bacterial modifications may be due in part to the bile sensitivity previously exhibited by MOL361 and/or the high abundance of Turicibacter in the small intestine, causing these bacteria to more strongly associate with host genes for bile reabsorption and lumenal bile levels than other bile-modifying gut bacteria^{21,53-57}.

This work displays metabolic consequences of colonization by specific gut bacteria and improves the resolution of our understanding connecting specific taxa—in this case, at the strain level—with host physiology. In rodent and human studies, Turicibacter relative abundance often negatively correlates with dietary fats^{29,31,58-62} and host adiposity^{28,33,63}, but some studies have shown opposite relationships^{30,64,65}. This could be a result of the phenotypic diversity identified here among Turicibacter isolates wherein the host may experience different lipid outcomes depending on their own specific Turicibacter strains, but could also vary with other features such as host genetics and sex^{21,38}. Importantly, effects of Turicibacter colonization could also be influenced by biogeographic organization of an individual's microbiota; in addition to the specific taxonomic membership, Turicibacter positioning in the small and large intestine may affect host consequences from their respective bile modifications by transforming bile pools in either section of the gut tract. Despite genomic and localization differences between them, the study found that MOL361 and H121 induced lipid metabolite changes that indicate increases in fatty acid oxidation, suggesting that these strains at least share features that alter host fatty acid metabolism.

Further research into bsh gene regulation in the Turicibacter genus, the relationship between bile and Turicibacter colonization and transmission behaviors, as well as the native functionality of the putative BSH and 7α-HSDH homologs the study tested, will further explain how these bacteria wields their bile modifications in the intestine. Interestingly, several of the in vitro bile transformations, specifically increases in unconjugated bile acids, were generalizable between our in vitro characterizations and Turicibacter colonization, whereas concurrent in vivo increases in certain conjugated bile acids seen during 1E2 and H121 colonization indicate the possibility of more complex interactions between host bile production and Turicibacter colonization. The fact that the specificity and activity of the individual BSH homologs differed when expressed in different bacterial backgrounds indicates that other unknown cellular or environmental factors influence the way individual BSH act in vivo. This may also include mechanisms that modify the functionality of proteins encoded by the other putative BSH homologs the study identified from Turicibacter which did not deconjugate the specific tauro- or glyco-conjugated bile acids used in our experiments. These findings may influence the ways that bile-modifying genes can be employed to shape host lipid profiles through microbiota engineering, positioning certain microbial species as more appropriate vectors to impart specific host effects. It will also be informative to determine what other activities performed by Turicibacter lead to WAT gain in colonized animals, which contrasted findings from colonizing mice with specific bsh-overexpressing strains of B. thetaiotaomicron. Given this finding, it is likely that Turicibacter also influences host lipids through other mechanisms in addition to the bile transformations the study characterized.

Our work also connects specific Turicibacter members and BSH activity with specific host outcomes. Though some host responses such as broadly decreased triglycerides were consistent across our BSH-recipient mice, the exact lipid and cholesterol responses differed, indicating that the type of deconjugations might have differing connections with host physiology. Further work will continue strengthening the exciting prospect of utilizing Turicibacter and/or its bile modifications to intentionally alter host lipid biology to improve host metabolic and lipid-associated health^{66,67}, as has been proposed with other bacteria^{54,68}. Beyond lipid biology, Turicibacter abundance has been positively correlated with diseases such as Parkinson's disease⁶⁹ and depression⁷⁰, and selective serotonin reuptake inhibitors (SSRIs) have been found to negatively affect Turicibacter growth and colonization⁷¹, potentially because they inhibit activity of its unique serotonin transporter³⁸. SSRI use is frequently associated with metabolic side effects like weight gain^{72,73}, and our findings suggests a hypothesis that connects SSRI use and these side effects: SSRI use could diminish gut colonization of bacteria like Turicibacter, thus unintentionally altering their impact on host physiology. Future work may develop strategies to reduce interactions between SSRIs and activity of the microbiota, and minimizing the side effects of these drugs and improving host outcomes. In all, these associations further emphasize the importance of understanding mechanisms connecting members of the diverse Turicibacter genus to host physiology.

### Methods

### Mouse Husbandry

Adult (6-8 week old) germ-free Swiss Webster mice were used for all animal experiments according to UCLA Institutional Animal Care and Use Committee-approved protocols. Mice were reared in flexible gnotobiotic isolators on a 12 h: 12 h light dark schedule on standard chow (Labdiet 5K52, 22.1%: 16.6%: 61.3% protein: fat: carbohydrate by calories), then were exited to autoclaved filter top cages with autoclaved chow (Labdiet 5010, 28.7%: 13.1%: 58.2% protein: fat: carbohydrate by calories) and water. After one day of cage acclimation, the noted Turicibacter or Bacteroides thetaiotaomicron strain was grown in YCFA medium (see below) overnight, pelleted by centrifugation, and resuspended in 1×PBS. Mice were colonized by a 200 μL gavage containing ˜106 colony-forming units (CFU) of Turicibacter or ˜10⁸ CFU of B. thetaiotaomicron. Alternatively, mice were gavaged with the same volume of PBS alone (referred to as germ-free [GF]) or PBS-suspended fecal slurry from a specific pathogen-free adult mouse (referred to as conventionalized [CONV]). Colonization was quantified using strain-specific TuriSERT primers (Table 7) and quantitative PCR (qPCR) from weight-normalized contents from the distal small intestine and proximal colon after addition of Low Abundance Microbiota Standard (Zymo) and extraction using the Zymo DNA Mini kit (Zymo).

### Bacterial Culturing

Turicibacter isolates and Bacteroides thetaiotaomicron strains (Table 5) were cultured in a flexible vinyl chamber (Coy) in an anaerobic 85%/10%/5% nitrogen/carbon dioxide/hydrogen mixture (Airgas). Turicibacter was grown on Schaedler's agar (BD Biosciences) or modified YCFA74 (pH 7.4, per liter: 100 mM MOPS, 10 g casitone, 2.5 g yeast extract, 2 g glucose, 2 g maltose monohydrate, 2 g cellobiose, 44 mg MgSO₄, 68 mg CaCl₂), 0.9 g NaCl, 10 mg hemin, 0.45 g K₂HPO₄, 0.45 g KH₂PO₄, 4 g NaHCO₃, 1 g cysteine, 1 mg resazurin, 1.9 mL glacial acetic acid, 0.7 mL propionic acid, 90 μL isobutyric acid, 100 μL isovaleric acid, 100 μL valeric acid, 10 mL ATCC vitamin mixture, 0.2% Tween-80) at 37° C. Cells were normally grown without shaking, but when appropriate, Turicibacter cultures were anaerobically transferred to sealed Hungate tubes or 1.7 mL microcentrifuge tubes and shaken at 225 RPM at 37° C.

For B. thetaiotaomicron growth curves, overnight cultures were grown anacrobically in BHI-S for ˜48 hours at 37° C. to ensure culture saturation, then were subcultured 1:50 for 6 hours at 37° C. (final OD₆₀₀=0.41-0.51). Subcultures were all then diluted to OD₆₀₀=0.1, and then six replicates were further diluted 1:10 in 100 μL BHI-S in a 96 well plate. Plates were anaerobically sealed with parafilm and incubated at 37° C. OD₆₀₀ readings were taken every 15 minutes in a Biotek Synergy H1 microplate reader (Agilent).

Escherichia coli C41-pLys (Lucigen) was used for characterizing putative bile modification genes, which were expressed off the pET21+plasmid. E. coli was grown acrobically shaking at 37° C. C in Luria Broth (LB, 1% NaCl, 1% tryptone, 0.5% yeast extract) supplemented with 100 μg mL⁻¹ ampicillin. Expression of genes was induced by addition of 100 μM IPTG.

### Bacterial Isolation and Identification

The frozen stool sample was thawed on ice and diluted 1:10 with PRAS anaerobic dilution blank medium (Anaerobe Systems). 100 μL of the diluted stool was further diluted to 1:1000 with modified YCFA media containing 0.05% bovine bile, 0.2% Tween-80, and 50 mM resorufin and loaded on Prospector® system arrays (Isolation Bio, San Carlos, CA, USA) following manufacturer's instructions. The fluorescent green signal of the arrays at time 0 was read on the Prospector® instrument in a Coy anaerobic chamber and the arrays incubated at 37° C. in an Anaerobic Systems AS-580 anaerobic chamber (Anacrobe Systems). At 17 and 41 hours of incubation the arrays were scanned again and the decrease in green fluorescence from time 0 was used as an indicator for bacterial growth in the array nanowells. Bacteria from the array were transferred to 96-well transfer plates containing 200 μL per well of modified YCFA media, without the addition of 50 mM resorufin. The transfer plates were sealed with a gas permeable film and incubated at 37° C. in a Mitsubishi AnaeroPack jar with a gas-generating sachet (Remel) for seven days. After incubation, the contents of 538 wells from the transfer plates with visible turbidity were consolidated into secondary 96-well plates, preserved with reduced glycerol, and stored at −80° C. until needed. Unless stated otherwise, all stool and isolate manipulations were conducted anaerobically with a 5% CO₂/5% H₂/90% N₂ atmosphere.

Genomic DNA was extracted in a 96-well format from the consolidated Prospector® culture plates using the Extract All Kit (Applied Biosystems). 20 μL of culture was combined with 20 μL of Lysis Solution and incubated for 10 minutes at 95° C., followed by three minutes at room temperature. The DNA was stabilized with the addition of 20 μL of DNA Stabilizing Solution and the resulting DNA lysate stored at −20° C. until needed.

qPCR Screening of Novel Turicibacter Isolates

Genomic DNA from 538 isolates was screened for Turicibacter 16S and the Turicibacter TuriSERT38 gene using a multiplexed primer set (Table 7). Each 25 μL qPCR reaction mixture had 1 μL Extract All lysate, 10 μL SYBR Power master mix (Applied Biosystems), 0.5 μL of each of the 10 μM primers, and 12 μL molecular grade water. The reactions were run in a QuantStudio 6 Flex (Thermo Fisher) with a 95° C. hold followed by 40 cycles of 95° C. for 15 s, 50° C. for 30 s, 72° C. for 30 s. Turicibacter sanguinis MOL361 gDNA and water were used as positive and negative controls, respectively.

### Molecular Cloning

Turicibacter genes were amplified from template culture lysates with Phusion or Q5 DNA polymerase (NEB) and primers designed to amplify denoted Turicibacter genes. pET21- or pWW383748 derived expression plasmids were assembled using Gibson assembly (see Table 7 for oligos) for expression in E. coli or Bacteroides thetaiotaomicron, respectively. Cloned constructs were confirmed through Sanger sequencing prior to functional characterization. pWW3837-derived constructs were cloned into B. thetaiotaomicron VPI-5482 as previously described⁴⁸. bsh-expressing B. thetaiotaomicron was compared to B. thetaiotaomicron containing the original pWW3837 construct (referred to as wild-type B. thetaiotaomicron).

### Genome Assemblies

Each strain was streaked on Schaedler agar plates and incubated anaerobically, then an individual colony from each isolate was picked into YCFA medium and grown overnight at 37° C. DNA was extracted using the Zymo DNA mini kit (Zymo), with bead beating used to lyse cells. Purified genomic DNA was sequenced by MiGS (migscenter.org), and 151 bp paired-end sequences were assembled using CLC Genomics Workbench (Qiagen). Genome assemblies have been deposited at NCBI at BioProject PRJNA846348.

### Whole Genome and Gene Comparisons

anvi'o⁷⁵ was used to profile and visualize the different Turicibacter strain DNA sequences to locate putative bile salt hydrolase and 7α-HSDH homologs in contig groups, generate variability profiles, and measure gene coverage and detection statistics. Average nucleotide identity (ANI) was calculated using OrthoANIu⁷⁶ (available https://www.ezbiocloud.net/tools/ani).

Sequences comparisons between 16S rRNA and bsh genes/BSH amino acid sequences were performed in CLC Genomics Workbench (Qiagen). 7α-HSDH sequence comparisons were performed using tblastn⁷⁷ using the translated amino acid sequence from Clostridium absonum⁴⁴.

### Assessment of Bile Transformations

In vitro characterization of bile transformations by engineered E. coli or B. thetaiotaomicron strains were performed by growing cells in respective media conditions described above supplemented with 0.5 mM (total combined concentration) of the noted bile species. Cells were grown to stationary phase (shaking at 37° C.), then frozen at −80° C. until further processing. Cells were then thawed, pelleted (5 minutes at 16,000×g), and the supernatant was removed to a new microcentrifuge tube. Three volumes of methanol was added, then the mixture was vigorously mixed for 30-60 seconds and incubated (room temperature, 15 minutes). Mixtures were centrifuged (5 minutes, 16,000×g), the supernatant removed to a clean microcentrifuge tube and dried in a vacuum concentrator. The dried residue was treated with methanol/water/formic acid (50/50/0.1, all by volume) then vigorously mixed and centrifuged as described above. Supernatants were transferred to polypropylene HPLC vials, capped, and maintained at 4° C. while aliquots (typically 5 μL) were injected onto a reversed phase HPLC column (Cadenza CD-C18, 3.0 μm, 250×2 mm, Imtakt) equilibrated in solution A (water/formic acid, 100/0.1, vol./vol.) and eluted (0.2 mL minute-1) with an increasing concentration of solution B (acetonitrile/formic acid, 100/0.1, vol./vol.); minute/% B: 0/30, 45/70, 48/100, 50/30, 67/30). The effluent from the column was passed through an electrospray ion source (capillary voltage 42V, capillary temperature 275° C., sheath gas flow 15 L min⁻¹, spray voltage 5 kV, and −15 kV conversion dynode with −1.2 k V multipliers) connected to a linear ion trap mass spectrometer (Thermo LTQ) scanning from m/z 95-1000 in the positive ion mode. Spectra were recorded and analyzed with instrument-manufacturer supplied software. Confirmation of proposed elemental compositions was achieved using the same chromatography and ion source configuration with the spectra recorded by scanning on an orbitrap mass spectrometer (Thermo LTQ XL).

For bile species quantification, an internal spike-in standard of 100 mM chenodeoxycholic acid-D4 (CDCA-D4, Sigma) was added to initial culture supernatants as a normalization reference. Area under the curve from reconstructed ion chromatograms was used to quantify the abundances of each species.

### SERUM Metabolite Analysis

Mice were euthanized with isoflurane and whole blood was collected via cardiac puncture. Blood was allowed to clot in SST Vacutainer tubes (BD) on ice, then centrifuged (4° C., 1 minute, 1500×g). The supernatant was removed and snap frozen in liquid nitrogen. Serum metabolites were analyzed using global metabolomics platform by Metabolon (Morrisville, NC, USA). Unless otherwise noted, values presented are in arbitrary units (a.u.) for that particular metabolite, determined by the log-transform of the volume-corrected quantification.

### Circulating Lipid Analysis

Mice were fasted for 4-6 hours, then euthanized as described above. Blood was collected via cardiac puncture and deposited into anticoagulatory K₂EDTA Vacutainer tubes (BD) on ice. Blood was centrifuged (4° C., 15 minutes, 2000×g), then plasma was collected from supernatant and snap frozen in liquid nitrogen. Shotgun lipidomics was performed by the UCLA Lipidomics Core (Los Angeles, CA, USA) with the following protocol. Thawed plasma was pipetted into glass tubes, a mixture of 70 internal standard lipids (Sciex and Avanti) was added, and lipids were extracted using a modified Bligh and Dyer extraction⁷⁹. The pooled organic layers from two extractions were dried in a vacuum concentrator and resuspended in 50/50 (vol./vol.) methanol/dichloromethane plus 10 mM ammonium acetate. After transfer to robovials, samples were analyzed with a Sciex 5500 with DMS Device (Lipidyzer Platform) using a targeted acquisition list of 1450 lipid species. The Lipidyzer Differential Mobility device was tuned using the EquiSPLASH LIPIDOMIX standard mixture (Avanti). Data was analyzed using an in-house platform using previously described parameters⁸⁰, and quantitative values were normalized to input volume. Statistical significance identification for species to include in heatmap was performed with two-tailed Welch's t-test (p-value cutoff <0.05).

### Total Colonic Bile Concentration Measurement

Mice were colonized and fasted as described above, then upon sacrifice contents from ˜ 1 cm of proximal colon were collected into microcentrifuge tubes and snap frozen in liquid nitrogen. Thawed contents were weighed and suspended into water, then total bile levels were measured using the Bile Acid Assay Kit (Sigma). Total bile values were normalized by sample mass, and each sample value was compared to sex-matched littermate controls. Values shown at “0” were below limit of detection.

### Adipocyte Area Calculation

After sacrifice, mice epidydimal or gonadal white adipose tissue (e/g WAT) pads were weighed and placed into 4% paraformaldehyde in 1×PBS for 48 hours at 4° C. Fat pads were washed twice in 70% ethanol, then submitted to the UCLA Translational Pathology Core Laboratory (Los Angeles, CA, USA) for paraffin embedding, sectioning, and H&E staining. Ten adipocyte images from each animal (five from each fat pad) were visualized with a 20× objective on an EVOS microscope (Thermo). Adipocyte area for all cells contained entirely within the field of view was automatically measured using the Fiji⁷⁸ Adiposoft⁷⁹ plug-in (version 1.1.16).

qRT-PCR Measurement of Liver Transcripts

Gnotobiotic mice were colonized with single as described above, and upon sacrifice, the median lobe of the liver was dissected and either directly snap frozen in liquid nitrogen (all Turicibacter colonized animals) or placed in Trizol, bead bead for one minute, then frozen in liquid nitrogen (all B. thetaiotaomicron colonized animals). All livers were then transferred to −80° C. until further processing. Directly snap frozen livers were thawed overnight at −20° C. in RNALater-ICE (ThermoFisher), then bead beat in Trizol for one minute, after which all samples were processed in the same manner. RNA was extracted from thawed Trizol samples using the Direct-Zol RNA Miniprep Kit (Zymo), then cDNA was generated using the qScript cDNA Synthesis Kit (Quantabio). qPCR was performed using the PowerUp SYBR Green Master Mix (ThermoFisher) on a QuantStudio5 Real-Time PCR System (ThermoFisher) (primers^{83,84} available in Table 7) (cycling conditions: 50° C. for 2 min, 95° C. for 2 min, 50 cycles of 95° C. for 15 sec, 55° C. for 15 sec, 72° C. for 1 min, followed by melt curve. Fold changes in comparison with sex-matched controls (GF for Turicibacter colonizations, Bt-WT for B. thetaiotaomicron colonizations) were calculated using the AACt method with auto-thresholded Ct values with ppia as the house-keeping gene.

### Statistical Analysis

Statistical calculations were performed in in Prism 9.3.1 (Graphpad). Unless otherwise noted, ***=p<0.0005, **=p<0.005, *=p<0.05, written p-value=0.05<0.2. Heatmaps were created using the pheatmap⁸⁰ package in R (version 3.6.3)⁸¹.

Table 7 shows a list of oligos used in the work.
Table 8 shows a list of strains used in this work.
Table 9 shows serum metabolomics from GF, CONV, and Turicibacter monocolonized mice. Volume-adjusted log-transformed levels of listed serum metabolites from mice from different colonization states. Note that statistics on far right of sheet are generated automatically as part of analysis pipeline, but were not used for this work because data analyzed did not have normal distribution.
Table 10 shows absolute quantification of lipid species from plasma of mice colonized with B. thetaiotaomicron engineered to express Turicibacter bsh genes.

TABLE 7

| | SEQ | | | |
|---|---|---|---|---|
| | ID | | | |
| Name | NO: | sequence | purpose | Reference |
| TuriSERT-H121_F | 19 | GGTTTAGCTGATGCTGGAATTAG | Turicibacter | this study |
| | | | discovery/qPCR | |
| TuriSERT-H121_R | 20 | TCCAAATTTATCAACAAATGCTGTAATAAT | Turicibacter | this study |
| | | | discovery/qPCR | |
| TuriSERT- | 21 | GGGTTTGCAGATGCGGG | Turicibacter | this study |
| MOL361_F | | | discovery/qPCR | |
| TuriSERT- | 22 | AATTTATCAACCACCGCTGTAATAAT | Turicibacter | this study |
| MOL361_R | | | discovery/qPCR | |
| TuriSERT-1E2_F | 23 | GGGTTTGCTGATGCCGC | Turicibacter | this study |
| | | | discovery/qPCR | |
| TuriSERT-1E2_R | 24 | CCCAAATTTATCAACTACTGCTGTAATAAT | Turicibacter | this study |
| | | | discovery/qPCR | |
| pET21_F | 25 | CTCGAGCACCACCACCAC | linearize pET21 | this study |
| | | | E. coli | |
| | | | expression vector | |
| pET21_R | 26 | GAATACCTCCTTAAGTTGTTCTAGAAGG | linearize pET21 | this study |
| | | | E. coli | |
| | | | expression vector | |
| BSHIMOL361_F | 27 | aacaacttaaggaggtattcATGTGTACAGGACTTAGC | insert into pET21 | this study |
| BSHIMOL361_R | 28 | tggtggtggtggtgctcgagGTTTTGGAATAAAATATCTTG | insert into pET21 | this study |
| | | TTTACCTTG | | |
| BSHIIH121_F | 29 | aacaacttaaggaggtattcATGTGTACAGGGTTAAGTTTA | insert into pET21 | this study |
| | | AC | | |
| BSHIIH121_R | 30 | tggtggtggtggtgctcgagGTTTTGGAACAAGATATCTTG | insert into pET21 | this study |
| | | CTCTCC | | |
| BSHIII1E2_F | 31 | aacaacttaaggaggtattcATGTGCACAGGATTATGC | insert into pET21 | this study |
| BSHIII1E2_R | 32 | tggtggtggtggtgctcgagATTTTGATAACAAATATCTTG | insert into pET21 | this study |
| | | TTTTCCTAGG | | |
| BSHIVMOL361_F | 33 | aacaacttaaggaggtattcATGTGTACAGCATTATCATTA | insert into pET21 | this study |
| | | AC | | |
| BSHIVMOL361_R | 34 | tggtggtggtggtgctcgagATTTTGTAAATTTACAGCAAA | insert into pET21 | this study |
| | | TTCATCATTA | | |
| BSHVMOL361_F | 35 | aacaacttaaggaggtattcATGTGTACAGCTATTACATTA | insert into pET21 | this study |
| | | AAG | | |
| BSHVMOL361_R | 36 | tggtggtggtggtgctcgagATTTTGTTTCTGTACTACCAG | insert into pET21 | this study |
| | | TTTATCT | | |
| BSHVIH121_F | 37 | aacaacttaaggaggtattcATGTGTACAGCCATTACATTA | insert into pET21 | this study |
| | | AAAAC | | |
| BSHVIH121_R | 38 | tggtggtggtggtgctcgagATTCTGATCATGGACTGTTAA | insert into pET21 | this study |
| | | TCTTGT | | |
| BSHVII1E2_F | 39 | aacaacttaaggaggtattcATGTGTACAGCAATCTCAATC | insert into pET21 | this study |
| BSHVII1E2_R | 40 | tggtggtggtggtgctcgagCTCAAAAAAAGTCAGCGTCTT | insert into pET21 | this study |
| | | GC | | |
| BSHVIIIMOL361_F | 41 | aacaacttaaggaggtattcATGAAGGAAGGAACCATC | insert into pET21 | this study |
| BSHVIIIMOL361_R | 42 | tggtggtggtggtgctcgagATTAAGATATTTGATGTCATT | insert into pET21 | this study |
| | | TATAGGATTTAATTCG | | |
| 7A_HSDS_F | 43 | aacaacttaaggaggtattcATGCGAAAATTAGAGAATGC | insert into pET21 | this study |
| | | AATAGC | | |
| 7A_HSDS_R | 44 | tggtggtggtggtgctcgagCTTTTCCTCAACTACTTTACG | insert into pET21 | this study |
| | | ACC | | |
| pWW3837_fwd | 45 | GGATCTGATTACAAGGAC | linearize pWW3837 | this study |
| | | | B. thetaiotaomicron | |
| | | | overexpression | |
| | | | vector | |
| pWW3837_rev | 46 | TATTTTAAATTTTTTTGAATCCATAGATC | linearize pWW3837 | this study |
| | | | B. thetaiotaomicron | |
| | | | overexpression | |
| | | | vector | |
| pWW_BSHIVMOL361_F | 47 | tggattcaaaaaaatttaaaataATGTGTACAGCATTATCATT | insert into pWW3837 | this study |
| | | AAC | | |
| pWW_BSHIVMOL361_R | 48 | tcgtccttgtaatcagatccATTTTGTAAATTTACAGCAAAT | insert into pWW3837 | this study |
| | | TC | | |
| pWW_BSHIMOL361_F | 49 | tggattcaaaaaaatttaaaataATGTGTACAGGACTTAGC | insert into pWW3837 | this study |
| pWW_BSHIMOL361_R | 50 | tcgtccttgtaatcagatccGTTTTGGAATAAAATATCTTGT | insert into pWW3837 | this study |
| | | TTAC | | |
| pWW_BSHIIIIE2_F | 51 | tggattcaaaaaaatttaaaataATGTGCACAGGATTATGC | insert into pWW3837 | this study |
| pWW_BSHIIIIE2_R | 52 | tcgtccttgtaatcagatccATTTTGATAACAAATATCTTGT | insert into pWW3837 | this study |
| | | TTTC | | |
| pWW_BSHIIH121_F | 53 | tggattcaaaaaaatttaaaataATGTGTACAGGGTTAAGTTT | insert into pWW3837 | this study |
| | | AAC | | |
| pWW_BSHIIH121_R | 54 | tcgtccttgtaatcagatccGTTTTGGAACAAGATATCTTG | insert into pWW3837 | this study |
| ppia_F | 55 | CAGTGCTCAGAGCTCGAAAGT | baseline qRT-PCR | Takahashi, |
| | | | control | 2016 |
| ppia_R | 56 | GTGTTCTTCGACATCACGGC | baseline qRT-PCR | Takahashi, |
| | | | control | 2016 |
| Fxr_F | 57 | TCCAGGGTTTCAGACACTGG | fxr transcript | Sayin, |
| | | | measurement | 2013 |
| Fxr R | 58 | GCCGAACGAAGAAACATGG | fxr transcript | Sayin, |
| | | | measurement | 2013 |
| Cyp7a1_F | 59 | AGCAACTAAACAACCTGCCAGTACTA | Cyp7al transcript | Sayin, |
| | | | measurement | 2013 |
| Cyp7a1_R | 60 | GTCCGGATATTCAAGGATGCA | Cyp7al transcript | Sayin, |
| | | | measurement | 2013 |

TABLE 8

| | Derived | | | |
|---|---|---|---|---|
| Isolate | from | Source | Reference | Notes |
| MOL361 | human | DMSZ | Bosshard, | Turicibacter |
| | | | 2002 | sanguinis type strain |
| 18F6 | human | Kenya Honda | N/A | |
| T46 | human | Thomas Auchtung | N/A | |
| GALT-G1 | human | this work | N/A | |
| H121 | contaminated | Thomas Auchtung | Auchtung, | |
| | germ-free | | 2016 | |
| | mouse | | | |
| T129 | human | Thomas Auchtung | N/A | |
| GALT-E2 | human | this work | N/A | |
| 1E2 (also known as 80E2) | mouse | Kenya Honda | N/A | |
| TA25 | mouse | Thomas Auchtung | N/A | |
| Bacteroides | human | Justin | Xu, 2003 | background strain |
| thetaiotaomicron | | Sonnenburg/Fatima | | |
| VPI-5482 | | Enam | | |
| B. | N/A | rederived for | Whitaker, | referred to as “WT” |
| thetaiotaomicron pWW3837 | | this work | 2017 | in this work |
| Bt-BSH-IV: MOL361 | N/A | this work | N/A | WT B. |
| | | | | thetaiotaomicron |
| | | | | with BSH-IV from |
| | | | | MOL361 in place of |
| | | | | GFP |
| Bt-BSH-I: MOL361 | N/A | this work | N/A | WT B. |
| | | | | thetaiotaomicron |
| | | | | with BSH-I from |
| | | | | MOL361 in place of |
| | | | | GFP |
| Bt-BSH-II: H121 | N/A | this work | N/A | WT B. |
| | | | | thetaiotaomicron |
| | | | | with BSH-II from |
| | | | | H121 in place of |
| | | | | GFP |
| Bt-BSH-III: 1E2 | N/A | this work | N/A | WT B. |
| | | | | thetaiotaomicron |
| | | | | with BSH-III from |
| | | | | 1E2 in place of |
| | | | | GFP |
| Escherichia coli C41-pLys | N/A | Lucigen | N/A | expression strain |
| Escherichia coli S17λ-pir | N/A | Justin | Lynch, | conjugation donor |
| | | Sonnenburg | 2012/Whitaker, | |
| | | | 2017 | |

TABLE 9
Fold Change
Welch's Two-Sample t-Test (bold indicates p < 0.1)

| MOL361 | 1E2 | H121 | T129 | CONV |
|---|---|---|---|---|
| GF | GF | GF | GF | GF |
| 0.66 | 1.07 | 1.14 | 1.06 | 0.57 |
| 0.66 | 1.13 | 1.01 | 1.08 | 0.36 |
| 0.82 | 0.93 | 1.78 | 1.05 | 0.39 |
| 1.00 | 1.10 | 0.79 | 1.18 | 1.04 |
| 1.55 | 1.76 | 1.93 | 1.47 | 2.63 |
| 1.27 | 1.32 | 1.44 | 1.11 | 1.56 |
| 1.08 | 1.17 | 1.58 | 1.02 | 0.92 |
| 0.94 | 1.08 | 1.20 | 1.02 | 1.36 |
| 1.06 | 1.39 | 1.26 | 1.16 | 1.17 |
| 0.82 | 0.98 | 0.95 | 0.99 | 1.38 |
| 1.04 | 1.60 | 1.26 | 1.38 | 1.41 |
| 1.02 | 1.22 | 1.33 | 1.18 | 1.14 |
| 1.21 | 1.37 | 1.20 | 1.39 | 1.42 |
| 0.90 | 0.96 | 1.12 | 1.02 | 0.99 |
| 0.96 | 1.02 | 0.96 | 0.98 | 0.93 |
| 0.94 | 1.09 | 1.20 | 0.96 | 0.87 |
| 1.04 | 1.19 | 1.24 | 0.92 | 1.20 |
| 1.03 | 1.25 | 1.17 | 1.00 | 1.42 |
| 1.22 | 1.40 | 1.29 | 1.22 | 1.95 |
| 1.05 | 1.10 | 1.10 | 0.97 | 1.00 |
| 1.08 | 1.23 | 1.13 | 1.12 | 0.63 |
| 0.93 | 0.97 | 0.98 | 1.06 | 0.69 |
| 1.01 | 0.97 | 0.94 | 0.98 | 0.53 |
| 0.61 | 0.84 | 0.62 | 0.63 | 0.43 |
| 0.84 | 1.25 | 0.93 | 1.21 | 0.71 |
| 0.89 | 1.32 | 0.96 | 1.14 | 0.54 |
| 0.95 | 1.30 | 0.84 | 1.14 | 0.64 |
| 0.71 | 1.09 | 0.80 | 1.09 | 0.70 |
| 0.78 | 1.23 | 0.98 | 1.03 | 0.59 |
| 0.89 | 0.93 | 0.86 | 1.04 | 0.78 |
| 0.71 | 0.85 | 0.89 | 0.89 | 0.79 |
| 0.71 | 1.14 | 0.72 | 1.07 | 0.57 |
| 0.90 | 1.05 | 0.98 | 1.01 | 0.85 |
| 0.85 | 0.99 | 0.94 | 1.16 | 1.24 |
| 0.78 | 1.10 | 0.97 | 0.91 | 0.65 |
| 0.84 | 0.98 | 0.97 | 1.04 | 0.83 |
| 0.79 | 0.90 | 0.85 | 1.02 | 0.58 |
| 0.63 | 0.98 | 0.85 | 1.05 | 0.54 |
| 0.52 | 0.81 | 0.60 | 0.63 | 0.40 |
| 1.13 | 1.40 | 1.02 | 1.30 | 0.41 |
| 0.79 | 1.16 | 0.84 | 1.02 | 0.48 |
| 1.19 | 1.05 | 1.20 | 0.93 | 0.95 |
| 0.96 | 1.41 | 1.11 | 1.14 | 1.04 |
| 0.96 | 1.28 | 1.08 | 1.05 | 1.01 |
| 0.85 | 0.89 | 0.85 | 0.97 | 1.32 |
| 1.50 | 1.27 | 1.16 | 0.87 | 0.66 |
| 1.22 | 1.46 | 1.32 | 1.13 | 1.28 |
| 0.87 | 0.95 | 0.91 | 0.75 | 0.79 |
| 0.97 | 1.01 | 0.99 | 1.01 | 1.13 |
| 0.93 | 1.03 | 1.04 | 1.15 | 1.09 |
| 0.65 | 1.05 | 0.69 | 0.96 | 0.43 |
| 1.23 | 1.16 | 1.05 | 1.23 | 1.14 |
| 0.89 | 1.14 | 0.99 | 1.01 | 0.82 |
| 1.18 | 1.48 | 1.21 | 1.22 | 0.90 |
| 1.17 | 0.98 | 1.16 | 1.07 | 1.44 |
| 0.87 | 1.10 | 1.05 | 1.01 | 0.70 |
| 0.95 | 1.13 | 1.03 | 1.13 | 1.15 |
| 0.92 | 1.08 | 0.99 | 1.04 | 0.90 |
| 0.91 | 1.12 | 1.04 | 1.04 | 0.79 |
| 0.51 | 0.93 | 0.48 | 0.45 | 0.36 |
| 1.12 | 1.74 | 0.74 | 1.59 | 0.54 |
| 0.84 | 0.96 | 1.01 | 1.08 | 1.33 |
| 0.99 | 1.18 | 1.09 | 1.15 | 1.22 |
| 0.87 | 1.13 | 1.03 | 0.99 | 1.02 |
| 0.65 | 1.04 | 0.68 | 0.87 | 0.52 |
| 1.02 | 1.06 | 1.04 | 1.13 | 0.98 |
| 1.00 | 0.94 | 0.85 | 1.03 | 1.08 |
| 0.79 | 0.79 | 0.80 | 0.94 | 0.90 |
| 0.99 | 1.18 | 1.10 | 1.17 | 0.93 |
| 1.04 | 1.04 | 1.01 | 1.15 | 1.09 |
| 0.94 | 0.74 | 0.83 | 0.85 | 1.44 |
| 0.74 | 0.62 | 0.82 | 0.91 | 0.85 |
| 0.96 | 1.08 | 1.04 | 1.18 | 1.01 |
| 0.84 | 0.95 | 0.84 | 1.09 | 1.18 |
| 0.69 | 0.77 | 0.81 | 0.94 | 0.89 |
| 0.97 | 1.02 | 1.02 | 1.10 | 1.15 |
| 1.19 | 1.08 | 1.09 | 1.22 | 1.38 |
| 0.80 | 0.88 | 0.89 | 0.99 | 0.99 |
| 0.82 | 0.95 | 0.99 | 1.01 | 1.22 |
| 1.00 | 0.87 | 0.98 | 1.12 | 1.07 |
| 0.84 | 1.06 | 0.88 | 0.83 | 0.84 |
| 0.94 | 0.99 | 0.94 | 1.01 | 0.95 |
| 0.91 | 0.91 | 0.85 | 0.98 | 1.07 |
| 0.44 | 0.80 | 0.60 | 0.84 | 0.65 |
| 0.50 | 0.75 | 0.41 | 0.49 | 0.41 |
| 0.78 | 1.14 | 1.01 | 1.13 | 0.57 |
| 0.74 | 1.14 | 0.92 | 1.06 | 0.57 |
| 0.90 | 1.42 | 0.74 | 0.96 | 1.02 |
| 1.01 | 0.94 | 0.99 | 1.07 | 0.91 |
| 1.00 | 0.84 | 0.85 | 0.97 | 1.05 |
| 0.95 | 0.94 | 0.96 | 1.03 | 0.99 |
| 2.43 | 1.21 | 2.07 | 2.74 | 1.63 |
| 1.04 | 0.88 | 0.94 | 1.11 | 1.15 |
| 0.88 | 0.66 | 0.85 | 0.90 | 1.44 |
| 0.91 | 0.98 | 0.98 | 1.10 | 0.88 |
| 0.82 | 0.81 | 0.79 | 0.97 | 0.98 |
| 0.75 | 0.81 | 0.83 | 0.90 | 0.89 |
| 0.95 | 0.94 | 0.96 | 1.04 | 1.04 |
| 0.86 | 0.75 | 0.83 | 0.87 | 1.27 |
| 0.83 | 0.90 | 0.84 | 1.00 | 1.00 |
| 0.96 | 0.89 | 0.87 | 0.97 | 0.84 |
| 0.93 | 0.88 | 0.85 | 0.92 | 0.99 |
| 0.56 | 0.78 | 0.47 | 0.46 | 0.43 |
| 0.83 | 0.96 | 0.95 | 1.05 | 0.94 |
| 0.75 | 0.86 | 0.94 | 1.13 | 1.08 |
| 1.00 | 1.19 | 1.02 | 1.04 | 0.88 |
| 1.10 | 1.14 | 1.16 | 1.33 | 1.11 |
| 1.67 | 1.24 | 1.31 | 1.61 | 1.33 |
| 0.98 | 1.01 | 1.03 | 1.18 | 0.59 |
| 0.82 | 1.27 | 1.28 | 1.21 | 0.55 |
| 0.78 | 1.31 | 1.20 | 1.18 | 0.44 |
| 0.74 | 0.61 | 1.17 | 0.73 | 0.90 |
| 1.19 | 1.14 | 1.61 | 1.65 | 0.45 |
| 1.03 | 1.39 | 3.43 | 1.48 | 0.21 |
| 0.53 | 1.91 | 6.01 | 1.07 | 0.18 |
| 1.14 | 0.95 | 1.54 | 1.52 | 0.77 |
| 0.93 | 1.04 | 1.22 | 1.38 | 0.60 |
| 1.22 | 1.17 | 1.57 | 1.78 | 0.43 |
| 0.91 | 0.91 | 1.26 | 1.01 | 0.74 |
| 0.71 | 1.02 | 1.16 | 1.45 | 0.42 |
| 1.00 | 1.00 | 1.00 | 1.00 | 1.66 |
| 0.85 | 0.77 | 0.81 | 0.77 | 35.50 |
| 1.00 | 0.84 | 0.98 | 1.27 | 1.03 |
| 1.01 | 0.96 | 1.04 | 1.06 | 1.20 |
| 0.96 | 1.09 | 1.14 | 0.86 | 1.02 |
| 1.21 | 1.31 | 1.53 | 1.17 | 0.75 |
| 0.78 | 1.36 | 0.77 | 0.67 | 16.52 |
| 0.75 | 1.09 | 0.80 | 0.96 | 0.62 |
| 0.89 | 0.79 | 0.62 | 1.02 | 1.29 |
| 0.85 | 1.12 | 0.81 | 1.02 | 0.58 |
| 0.31 | 0.27 | 0.31 | 0.47 | 0.28 |
| 0.68 | 0.62 | 0.89 | 0.90 | 4.06 |
| 0.90 | 0.82 | 0.96 | 1.04 | 0.91 |
| 0.74 | 1.30 | 0.85 | 1.43 | 0.67 |
| 0.76 | 1.08 | 0.97 | 1.19 | 0.94 |
| 0.93 | 0.90 | 0.99 | 0.95 | 1.17 |
| 0.89 | 0.83 | 1.43 | 1.06 | 0.88 |
| 0.89 | 1.01 | 1.27 | 1.28 | 1.28 |
| 0.85 | 1.10 | 0.96 | 1.12 | 0.91 |
| 0.99 | 1.07 | 1.38 | 1.04 | 0.81 |
| 0.66 | 1.03 | 0.80 | 0.90 | 0.56 |
| 0.84 | 0.58 | 1.00 | 0.86 | 0.94 |
| 0.81 | 1.09 | 1.06 | 0.98 | 0.42 |
| 0.64 | 1.07 | 0.71 | 0.85 | 0.63 |
| 0.55 | 0.46 | 0.71 | 0.51 | 1.62 |
| 0.71 | 0.71 | 0.90 | 0.52 | 0.48 |
| 0.96 | 0.89 | 0.87 | 0.80 | 2.69 |
| 0.81 | 1.08 | 0.79 | 1.00 | 0.51 |
| 1.08 | 0.99 | 1.07 | 1.16 | 0.86 |
| 1.24 | 1.26 | 1.31 | 1.34 | 1.03 |
| 1.26 | 1.15 | 1.21 | 1.02 | 1.47 |
| 0.75 | 0.47 | 0.50 | 1.06 | 0.92 |
| 0.93 | 1.04 | 0.99 | 1.23 | 1.01 |
| 1.45 | 1.62 | 2.21 | 1.84 | 0.99 |
| 1.01 | 0.86 | 0.84 | 0.89 | 1.23 |
| 0.82 | 1.16 | 0.71 | 0.97 | 0.41 |
| 0.89 | 1.09 | 0.97 | 0.96 | 1.19 |
| 1.01 | 1.26 | 1.47 | 1.20 | 1.16 |
| 0.77 | 0.93 | 0.84 | 1.16 | 0.97 |
| 0.57 | 0.94 | 0.84 | 0.79 | 0.69 |
| 0.64 | 0.93 | 0.65 | 0.81 | 0.52 |
| 0.68 | 1.03 | 0.76 | 0.93 | 0.74 |
| 0.59 | 1.19 | 0.97 | 0.75 | 0.55 |
| 1.04 | 0.77 | 1.00 | 0.86 | 1.15 |
| 0.67 | 0.99 | 0.70 | 0.83 | 0.67 |
| 0.63 | 0.96 | 0.54 | 1.41 | 1.30 |
| 1.04 | 1.23 | 1.18 | 1.50 | 1.28 |
| 0.91 | 0.94 | 1.10 | 0.86 | 0.90 |
| 0.45 | 0.52 | 0.72 | 0.66 | 0.48 |
| 0.71 | 1.14 | 0.83 | 1.09 | 0.80 |
| 0.83 | 1.31 | 1.10 | 0.97 | 1.12 |
| 1.00 | 1.00 | 1.00 | 1.00 | 2.27 |
| 1.18 | 1.18 | 1.07 | 1.14 | 1.01 |
| 0.98 | 2.67 | 5.68 | 1.41 | 0.37 |
| 1.14 | 0.80 | 0.88 | 1.05 | 1.44 |
| 0.95 | 0.79 | 0.90 | 0.93 | 0.84 |
| 0.87 | 0.75 | 0.81 | 0.95 | 0.94 |
| 0.90 | 1.03 | 0.98 | 1.10 | 0.84 |
| 0.98 | 0.99 | 0.93 | 1.05 | 1.13 |
| 0.88 | 1.12 | 1.59 | 1.30 | 2.05 |
| 1.00 | 1.00 | 1.00 | 1.00 | 3.63 |
| 0.97 | 0.95 | 0.88 | 0.91 | 0.93 |
| 1.29 | 1.38 | 1.40 | 3.59 | 2.25 |
| 0.74 | 0.74 | 0.97 | 0.74 | 6.01 |
| 1.02 | 1.13 | 1.13 | 1.14 | 0.94 |
| 0.79 | 0.78 | 0.93 | 1.15 | 0.92 |
| 0.65 | 1.45 | 1.00 | 1.05 | 1.08 |
| 0.76 | 1.08 | 0.94 | 0.91 | 0.66 |
| 0.80 | 0.86 | 0.92 | 0.98 | 1.21 |
| 1.11 | 1.05 | 1.60 | 1.39 | 0.72 |
| 6.49 | 8.20 | 12.43 | 10.88 | 1.00 |
| 0.77 | 1.20 | 1.30 | 0.99 | 0.89 |
| 0.96 | 0.94 | 1.28 | 1.08 | 1.26 |
| 0.95 | 0.76 | 0.87 | 0.79 | 1.32 |
| 1.08 | 0.93 | 0.90 | 1.05 | 1.11 |
| 0.83 | 0.71 | 0.85 | 0.82 | 0.66 |
| 0.80 | 1.03 | 1.13 | 0.86 | 1.91 |
| 1.42 | 1.46 | 1.23 | 3.02 | 2.83 |
| 1.05 | 1.07 | 1.88 | 1.20 | 0.49 |
| 1.43 | 1.31 | 1.78 | 1.59 | 1.00 |
| 0.94 | 0.68 | 1.69 | 1.07 | 0.68 |
| 1.08 | 0.83 | 1.90 | 0.91 | 2.74 |
| 1.30 | 1.34 | 1.74 | 1.43 | 1.39 |
| 1.94 | 1.50 | 2.32 | 1.27 | 1.80 |
| 0.65 | 0.78 | 1.00 | 0.94 | 0.88 |
| 1.04 | 0.97 | 1.87 | 1.16 | 0.36 |
| 0.91 | 0.83 | 1.46 | 1.04 | 0.38 |
| 1.03 | 1.02 | 2.27 | 1.09 | 0.89 |
| 1.28 | 1.25 | 1.50 | 1.59 | 1.50 |
| 1.55 | 1.58 | 1.82 | 1.59 | 2.30 |
| 1.41 | 0.70 | 0.97 | 2.44 | 1.07 |
| 1.40 | 1.38 | 2.17 | 1.73 | 0.88 |
| 0.67 | 0.83 | 1.13 | 0.86 | 0.45 |
| 1.25 | 1.20 | 1.56 | 1.48 | 0.98 |
| 0.95 | 0.93 | 0.89 | 0.85 | 1.21 |
| 0.78 | 0.73 | 1.42 | 1.08 | 0.92 |
| 0.52 | 0.53 | 0.69 | 0.54 | 0.35 |
| 0.68 | 0.55 | 0.61 | 0.86 | 0.73 |
| 0.64 | 0.72 | 0.96 | 0.62 | 0.75 |
| 0.68 | 1.24 | 0.95 | 0.94 | 316.97 |
| 2.57 | 0.55 | 1.80 | 0.93 | 1.77 |
| 2.10 | 0.73 | 1.32 | 0.65 | 5.73 |
| 0.75 | 0.75 | 0.90 | 0.91 | 0.94 |
| 1.01 | 0.94 | 1.14 | 0.65 | 5.72 |
| 0.92 | 0.96 | 1.08 | 1.05 | 0.97 |
| 0.93 | 0.98 | 1.14 | 1.11 | 1.03 |
| 0.68 | 0.70 | 0.57 | 1.11 | 0.66 |
| 1.09 | 0.96 | 0.99 | 0.85 | 0.64 |
| 0.77 | 0.84 | 0.91 | 0.94 | 0.95 |
| 1.17 | 0.77 | 0.76 | 0.87 | 1.15 |
| 1.26 | 1.37 | 1.31 | 1.05 | 1.23 |
| 1.29 | 0.93 | 1.53 | 3.48 | 109.01 |
| 1.57 | 1.18 | 1.55 | 3.61 | 0.62 |
| 1.29 | 1.44 | 1.08 | 1.31 | 1.99 |
| 1.11 | 1.07 | 1.14 | 1.02 | 1.16 |
| 1.05 | 0.84 | 1.17 | 0.99 | 0.84 |
| 0.70 | 1.45 | 1.10 | 1.06 | 1.11 |
| 0.84 | 0.96 | 1.07 | 0.66 | 2.84 |
| 1.13 | 1.19 | 1.23 | 0.98 | 0.98 |
| 0.76 | 1.52 | 0.70 | 0.83 | 10.96 |
| 1.01 | 0.94 | 0.98 | 0.99 | 2.81 |
| 1.00 | 1.20 | 1.00 | 1.00 | 13.74 |
| 0.41 | 1.17 | 0.79 | 0.59 | 1.58 |
| 0.98 | 1.23 | 1.19 | 1.61 | 1.09 |
| 0.96 | 1.00 | 1.17 | 0.87 | 0.96 |
| 0.92 | 0.92 | 0.92 | 0.92 | 196.36 |
| 0.98 | 0.98 | 0.98 | 0.98 | 58.21 |
| 0.74 | 0.68 | 0.76 | 0.99 | 1.53 |
| 1.03 | 0.96 | 1.17 | 1.63 | 0.87 |
| 0.64 | 0.93 | 0.95 | 0.73 | 0.83 |
| 0.79 | 0.75 | 0.77 | 0.85 | 0.96 |
| 0.73 | 0.62 | 0.64 | 0.90 | 1.31 |
| 1.05 | 0.87 | 0.84 | 1.08 | 1.00 |
| 1.02 | 2.54 | 1.22 | 1.01 | 1.91 |
| 0.97 | 2.73 | 0.88 | 0.74 | 1.54 |
| 0.94 | 1.49 | 1.70 | 1.16 | 2.16 |
| 0.89 | 1.23 | 0.78 | 0.91 | 1.10 |
| 0.91 | 0.96 | 1.10 | 1.08 | 1.09 |
| 1.25 | 0.81 | 0.49 | 0.40 | 0.39 |
| 4.58 | 1.20 | 4.94 | 0.64 | 24.63 |
| 0.84 | 0.60 | 0.55 | 1.51 | 1.24 |
| 0.49 | 0.61 | 0.69 | 0.52 | 1.93 |
| 0.83 | 0.95 | 0.99 | 0.86 | 1.12 |
| 1.11 | 0.64 | 0.79 | 0.75 | 1.66 |
| 0.84 | 1.16 | 1.31 | 0.71 | 1.94 |
| 0.79 | 0.80 | 0.85 | 0.87 | 0.68 |
| 1.95 | 3.23 | 1.42 | 1.63 | 1.73 |
| 1.04 | 0.82 | 1.56 | 1.11 | 0.53 |
| 1.25 | 1.05 | 1.89 | 1.19 | 1.83 |
| 0.78 | 0.85 | 0.82 | 0.61 | 0.34 |
| 1.29 | 0.86 | 1.24 | 0.79 | 2.17 |
| 1.02 | 1.07 | 1.35 | 1.29 | 0.95 |
| 1.02 | 1.18 | 1.18 | 0.97 | 0.81 |
| 0.96 | 1.00 | 0.91 | 1.10 | 1.21 |
| 1.10 | 0.90 | 1.06 | 1.04 | 2.02 |
| 1.06 | 1.00 | 0.99 | 1.04 | 1.29 |
| 1.08 | 1.20 | 1.11 | 1.27 | 0.66 |
| 0.97 | 1.11 | 1.15 | 1.11 | 0.87 |
| 0.94 | 0.62 | 0.84 | 0.98 | 0.93 |
| 0.84 | 0.90 | 0.86 | 0.95 | 0.73 |
| 0.76 | 0.71 | 0.87 | 1.20 | 0.64 |
| 1.26 | 0.99 | 1.08 | 1.12 | 1.13 |
| 1.43 | 1.87 | 1.66 | 1.49 | 1.37 |
| 1.00 | 1.00 | 1.00 | 1.00 | 2.91 |
| 0.98 | 0.73 | 1.07 | 0.99 | 1.23 |
| 1.18 | 2.31 | 1.93 | 1.19 | 0.90 |
| 4.89 | 5.30 | 32.93 | 83.25 | 2.94 |
| 1.12 | 1.06 | 1.11 | 1.09 | 1.02 |
| 1.08 | 1.18 | 2.08 | 1.43 | 0.77 |
| 1.23 | 0.99 | 1.74 | 1.74 | 0.98 |
| 1.15 | 1.23 | 1.18 | 1.05 | 1.24 |
| 1.05 | 1.23 | 1.20 | 1.27 | 1.12 |
| 0.68 | 0.48 | 0.66 | 0.86 | 0.63 |
| 2.73 | 0.56 | 1.29 | 2.68 | 1.10 |
| 1.01 | 0.11 | 0.67 | 3.14 | 0.63 |
| 0.99 | 0.99 | 1.51 | 1.12 | 0.64 |
| 1.07 | 0.75 | 1.00 | 0.92 | 0.74 |
| 0.81 | 1.24 | 0.88 | 1.12 | 0.44 |
| 0.98 | 0.96 | 1.17 | 1.07 | 1.02 |
| 0.75 | 0.85 | 0.69 | 0.68 | 0.57 |
| 0.99 | 1.00 | 1.05 | 1.00 | 1.04 |
| 0.80 | 0.87 | 1.78 | 2.25 | 0.78 |
| 0.41 | 0.34 | 0.64 | 0.54 | 0.43 |
| 0.33 | 0.30 | 0.34 | 0.63 | 0.29 |
| 0.85 | 1.01 | 1.23 | 0.93 | 0.79 |
| 0.97 | 1.30 | 0.98 | 1.02 | 1.18 |
| 0.85 | 0.74 | 0.78 | 0.78 | 0.98 |
| 1.03 | 1.04 | 1.15 | 1.11 | 1.21 |
| 0.61 | 1.15 | 0.98 | 0.79 | 1.27 |
| 0.80 | 0.87 | 0.87 | 0.95 | 0.97 |
| 0.89 | 0.82 | 0.87 | 0.76 | 0.75 |
| 0.71 | 1.04 | 0.94 | 1.31 | 0.58 |
| 0.67 | 0.95 | 0.83 | 0.78 | 0.41 |
| 0.90 | 0.85 | 0.98 | 1.06 | 0.93 |
| 1.33 | 1.22 | 1.74 | 1.44 | 0.77 |
| 0.94 | 0.89 | 0.81 | 0.72 | 0.85 |
| 0.66 | 0.64 | 0.64 | 0.93 | 0.78 |
| 1.01 | 1.09 | 0.97 | 1.20 | 0.90 |
| 0.99 | 1.15 | 1.21 | 1.41 | 0.74 |
| 0.26 | 0.32 | 0.58 | 0.34 | 1.17 |
| 1.12 | 1.21 | 1.14 | 0.82 | 0.70 |
| 0.92 | 0.83 | 0.85 | 0.66 | 0.67 |
| 0.98 | 1.02 | 1.10 | 1.14 | 1.10 |
| 0.77 | 0.77 | 0.84 | 0.76 | 1.47 |
| 1.16 | 1.15 | 1.99 | 1.46 | 0.76 |
| 1.25 | 1.06 | 0.76 | 1.40 | 1.50 |
| 1.00 | 0.81 | 0.76 | 1.19 | 1.16 |
| 1.17 | 1.01 | 1.01 | 1.32 | 0.96 |
| 0.93 | 1.21 | 1.04 | 1.76 | 1.56 |
| 1.29 | 1.21 | 0.95 | 1.38 | 1.12 |
| 1.01 | 0.97 | 1.21 | 1.24 | 0.76 |
| 1.05 | 1.02 | 1.06 | 0.95 | 0.90 |
| 3.85 | 5.20 | 15.92 | 53.03 | 0.47 |
| 0.91 | 0.75 | 0.77 | 0.91 | 1.41 |
| 1.21 | 1.29 | 1.23 | 1.12 | 1.10 |
| 0.84 | 1.02 | 1.01 | 1.03 | 1.10 |
| 0.45 | 0.54 | 1.04 | 0.56 | 2.10 |
| 0.89 | 0.93 | 1.19 | 1.04 | 0.95 |
| 0.87 | 0.88 | 1.10 | 1.03 | 1.02 |
| 0.83 | 0.82 | 1.07 | 0.86 | 0.98 |
| 0.86 | 0.78 | 1.11 | 0.77 | 0.98 |
| 0.90 | 0.78 | 1.11 | 0.74 | 0.96 |
| 1.01 | 1.05 | 1.06 | 1.02 | 0.98 |
| 0.96 | 0.99 | 1.07 | 1.07 | 1.25 |
| 0.84 | 1.04 | 1.08 | 1.16 | 3.75 |
| 0.66 | 1.01 | 1.21 | 1.35 | 0.53 |
| 1.28 | 1.35 | 1.80 | 1.57 | 0.58 |
| 1.00 | 0.79 | 1.41 | 0.84 | 1.34 |
| 1.00 | 1.24 | 1.45 | 1.13 | 2.41 |
| 1.25 | 1.38 | 1.04 | 0.80 | 5.44 |
| 0.72 | 0.58 | 0.42 | 0.79 | 4.62 |
| 1.28 | 1.30 | 1.33 | 1.28 | 1.10 |
| 0.68 | 1.20 | 0.88 | 0.68 | 0.76 |
| 0.92 | 0.79 | 0.85 | 0.92 | 1.14 |
| 1.10 | 1.04 | 1.57 | 1.21 | 0.87 |
| 0.96 | 0.99 | 1.34 | 1.16 | 3.62 |
| 1.05 | 1.05 | 1.54 | 1.28 | 2.49 |
| 0.94 | 1.03 | 1.07 | 1.17 | 0.21 |
| 1.23 | 1.35 | 1.27 | 1.08 | 0.93 |
| 1.14 | 0.77 | 1.14 | 0.63 | 1.18 |
| 1.17 | 1.28 | 1.53 | 0.82 | 1.12 |
| 0.86 | 1.30 | 1.56 | 0.56 | 0.99 |
| 1.00 | 1.00 | 1.00 | 1.00 | 1.26 |
| 1.16 | 0.83 | 1.19 | 0.74 | 167.54 |
| 1.19 | 1.04 | 0.93 | 1.58 | 1.44 |
| 1.03 | 0.64 | 0.81 | 0.87 | 1.34 |
| 1.12 | 1.08 | 0.94 | 1.13 | 1.15 |
| 2.99 | 5.14 | 6.81 | 6.61 | 0.52 |
| 1.63 | 2.10 | 2.08 | 1.81 | 1.09 |
| 16.37 | 27.62 | 35.33 | 28.37 | 1.70 |
| 0.88 | 0.89 | 0.92 | 0.96 | 0.99 |
| 0.97 | 1.13 | 1.08 | 1.10 | 0.43 |
| 0.91 | 0.87 | 0.90 | 1.09 | 1.08 |
| 0.99 | 0.77 | 0.87 | 0.88 | 0.79 |
| 0.91 | 0.95 | 0.89 | 1.35 | 9.54 |
| 1.03 | 0.73 | 0.68 | 7.87 | 75.87 |
| 1.21 | 1.23 | 3.26 | 1.67 | 1.01 |
| 1.31 | 1.05 | 1.81 | 1.17 | 1.13 |
| 0.89 | 1.01 | 1.78 | 1.22 | 0.58 |
| 1.37 | 1.21 | 1.81 | 1.34 | 1.15 |
| 0.99 | 1.34 | 1.21 | 1.38 | 1.07 |
| 0.82 | 0.88 | 0.76 | 1.13 | 0.76 |
| 0.96 | 1.27 | 1.10 | 1.17 | 0.79 |
| 1.26 | 2.29 | 1.80 | 1.33 | 1.21 |
| 1.02 | 1.25 | 1.16 | 1.23 | 1.10 |
| 0.96 | 1.10 | 1.03 | 1.17 | 1.02 |
| 1.04 | 1.09 | 1.33 | 1.02 | 1.28 |
| 1.00 | 1.26 | 1.11 | 1.41 | 1.34 |
| 1.13 | 1.02 | 1.02 | 0.89 | 1.23 |
| 0.85 | 0.88 | 0.87 | 0.83 | 1.48 |
| 1.09 | 1.02 | 0.92 | 0.88 | 2.87 |
| 1.49 | 1.24 | 1.00 | 0.82 | 2.11 |
| 1.16 | 1.23 | 1.09 | 0.98 | 2.14 |
| 1.03 | 1.06 | 0.98 | 1.08 | 0.97 |
| 1.35 | 1.05 | 1.34 | 1.68 | 0.74 |
| 0.93 | 1.04 | 0.86 | 1.06 | 1.25 |
| 0.81 | 1.01 | 0.87 | 0.96 | 0.93 |
| 0.94 | 1.23 | 1.23 | 1.10 | 1.63 |
| 0.41 | 0.55 | 0.85 | 0.87 | 0.77 |
| 0.66 | 1.02 | 1.05 | 1.16 | 0.79 |
| 1.26 | 1.11 | 3.05 | 0.97 | 0.88 |
| 1.25 | 1.05 | 1.73 | 1.48 | 2.19 |
| 0.84 | 0.77 | 0.84 | 0.92 | 0.68 |
| 1.00 | 1.00 | 1.00 | 1.00 | 4.29 |
| 0.91 | 0.50 | 0.85 | 1.17 | 1.33 |
| 0.80 | 1.47 | 1.09 | 1.18 | 0.46 |
| 0.77 | 1.26 | 0.90 | 0.99 | 0.42 |
| 1.21 | 1.08 | 1.68 | 1.42 | 0.84 |
| 1.36 | 1.06 | 1.46 | 1.64 | 1.06 |
| 0.99 | 0.82 | 0.97 | 0.76 | 1.27 |
| 0.68 | 15.80 | 0.30 | 0.63 | 1.31 |
| 0.38 | 0.26 | 0.26 | 0.78 | 0.62 |
| 0.16 | 0.10 | 0.08 | 0.55 | 0.29 |
| 1.29 | 0.95 | 0.78 | 0.83 | 1.26 |
| 1.45 | 1.56 | 2.08 | 1.73 | 0.90 |
| 0.84 | 0.79 | 0.80 | 0.65 | 4.06 |
| 0.98 | 0.98 | 0.98 | 1.05 | 0.67 |
| 0.94 | 0.94 | 0.94 | 0.88 | 0.81 |
| 0.77 | 1.14 | 1.06 | 1.23 | 0.77 |
| 0.80 | 1.86 | 1.01 | 1.67 | 0.58 |
| 0.74 | 0.91 | 1.20 | 1.49 | 0.84 |
| 0.83 | 1.10 | 0.92 | 0.98 | 0.51 |
| 0.96 | 0.81 | 0.75 | 0.68 | 1.18 |
| 0.77 | 1.33 | 0.95 | 1.19 | 0.69 |
| 0.79 | 0.86 | 0.84 | 1.03 | 0.31 |
| 1.32 | 0.89 | 1.94 | 1.55 | 1.08 |
| 0.77 | 1.90 | 1.07 | 1.40 | 0.49 |
| 0.95 | 1.75 | 0.81 | 1.70 | 0.38 |
| 1.00 | 0.97 | 1.85 | 1.27 | 0.55 |
| 1.22 | 1.22 | 2.24 | 1.46 | 0.82 |
| 0.83 | 1.42 | 1.84 | 1.87 | 1.36 |
| 0.97 | 1.05 | 1.26 | 0.86 | 0.82 |
| 0.90 | 1.31 | 0.99 | 1.28 | 1.00 |
| 0.76 | 0.75 | 1.04 | 1.47 | 0.85 |
| 0.68 | 0.80 | 0.95 | 0.83 | 0.43 |
| 1.07 | 1.19 | 1.90 | 1.03 | 1.38 |
| 0.77 | 1.09 | 1.09 | 1.63 | 0.56 |
| 1.34 | 1.11 | 1.46 | 2.11 | 1.25 |
| 1.00 | 1.00 | 1.00 | 1.00 | 1.83 |
| 1.00 | 1.00 | 1.00 | 1.00 | 1.46 |
| 1.00 | 1.00 | 1.00 | 1.00 | 1.36 |
| 1.06 | 1.09 | 0.95 | 1.24 | 0.81 |
| 0.80 | 1.32 | 1.27 | 2.02 | 0.71 |
| 1.08 | 1.18 | 1.01 | 1.22 | 1.22 |
| 1.01 | 0.95 | 0.97 | 0.99 | 1.00 |
| 0.82 | 1.39 | 1.00 | 1.55 | 0.91 |
| 1.08 | 1.64 | 1.35 | 1.44 | 0.81 |
| 1.08 | 1.29 | 1.15 | 0.83 | 4.92 |
| 0.77 | 1.29 | 0.83 | 0.75 | 0.84 |
| 0.83 | 0.89 | 0.75 | 0.80 | 0.99 |
| 0.64 | 0.69 | 1.16 | 0.73 | 1.49 |
| 0.93 | 0.73 | 0.69 | 0.70 | 0.95 |
| 0.77 | 0.90 | 0.72 | 1.13 | 0.47 |
| 1.02 | 1.04 | 0.96 | 1.11 | 1.04 |
| 1.65 | 1.84 | 2.10 | 1.49 | 0.95 |
| 0.95 | 0.92 | 1.00 | 1.00 | 1.68 |
| 0.69 | 0.74 | 1.01 | 0.91 | 0.83 |
| 0.95 | 0.96 | 1.37 | 2.42 | 1.53 |
| 0.76 | 0.93 | 1.08 | 0.77 | 1.12 |
| 1.01 | 1.05 | 0.87 | 0.97 | 2.89 |
| 0.99 | 1.05 | 0.96 | 1.11 | 2.16 |
| 0.90 | 0.91 | 0.86 | 0.96 | 2.07 |
| 0.94 | 0.98 | 1.00 | 0.81 | 1.85 |
| 0.80 | 0.96 | 0.89 | 0.96 | 1.57 |
| 0.80 | 0.91 | 0.93 | 0.92 | 1.87 |
| 0.84 | 0.86 | 0.94 | 0.76 | 2.57 |
| 0.83 | 1.07 | 1.06 | 1.02 | 1.62 |
| 1.01 | 0.96 | 1.05 | 0.90 | 1.73 |
| 0.97 | 1.12 | 1.09 | 1.06 | 1.88 |
| 0.91 | 1.04 | 1.14 | 0.91 | 2.07 |
| 0.85 | 0.94 | 0.96 | 0.88 | 1.82 |
| 0.80 | 0.91 | 0.90 | 0.95 | 1.71 |
| 0.90 | 0.75 | 0.79 | 0.95 | 1.11 |
| 0.71 | 1.05 | 0.95 | 1.17 | 1.03 |
| 0.85 | 1.50 | 1.55 | 1.37 | 1.65 |
| 0.59 | 1.18 | 1.22 | 1.28 | 0.68 |
| 0.88 | 1.04 | 0.91 | 0.82 | 2.25 |
| 1.19 | 1.04 | 1.13 | 1.04 | 1.22 |
| 0.95 | 0.88 | 0.83 | 0.87 | 0.97 |
| 1.37 | 1.05 | 1.61 | 3.27 | 0.62 |
| 0.98 | 1.02 | 0.99 | 0.89 | 2.07 |
| 0.87 | 0.88 | 1.64 | 1.01 | 3.44 |
| 1.01 | 0.99 | 1.05 | 1.11 | 1.40 |
| 0.95 | 0.95 | 0.93 | 1.07 | 1.03 |
| 0.88 | 1.28 | 1.98 | 1.07 | 0.98 |
| 0.80 | 0.75 | 0.81 | 0.74 | 0.87 |
| 1.26 | 0.99 | 1.15 | 0.80 | 0.37 |
| 1.00 | 1.00 | 1.04 | 0.99 | 2.40 |
| 1.00 | 0.98 | 1.04 | 1.00 | 1.12 |
| 0.97 | 0.91 | 1.06 | 1.14 | 0.89 |
| 0.87 | 0.74 | 0.91 | 1.01 | 1.83 |
| 0.92 | 0.90 | 0.79 | 0.88 | 0.58 |
| 0.81 | 0.72 | 0.94 | 0.93 | 1.57 |
| 0.70 | 0.66 | 0.64 | 0.45 | 0.57 |
| 0.93 | 0.91 | 0.91 | 0.96 | 0.87 |
| 1.15 | 1.14 | 1.10 | 1.16 | 1.07 |
| 0.90 | 0.93 | 0.84 | 1.07 | 1.04 |
| 1.24 | 0.90 | 0.75 | 1.44 | 1.09 |
| 0.96 | 0.63 | 0.92 | 1.31 | 1.76 |
| 0.70 | 0.98 | 1.25 | 0.90 | 0.64 |
| 4.41 | 3.13 | 9.47 | 3.13 | 0.79 |
| 6.63 | 6.25 | 15.63 | 3.56 | 0.87 |
| 0.95 | 0.80 | 1.17 | 0.90 | 1.55 |
| 1.56 | 1.26 | 0.97 | 1.56 | 0.89 |
| 1.24 | 0.79 | 0.90 | 0.93 | 1.14 |
| 1.35 | 1.20 | 1.11 | 0.77 | 0.82 |
| 1.38 | 1.13 | 0.96 | 1.02 | 1.00 |
| 0.99 | 0.87 | 0.64 | 1.10 | 0.85 |
| 1.35 | 0.99 | 0.72 | 1.14 | 1.15 |
| 1.36 | 1.09 | 0.97 | 0.97 | 1.08 |
| 1.27 | 0.88 | 1.01 | 0.76 | 1.21 |
| 1.75 | 0.69 | 1.18 | 0.39 | 1.66 |
| 1.06 | 1.12 | 0.71 | 1.13 | 1.22 |
| 0.44 | 0.37 | 0.61 | 0.58 | 2.95 |
| 1.03 | 1.05 | 1.38 | 2.32 | 0.73 |
| 0.64 | 0.48 | 0.69 | 0.68 | 0.86 |
| 0.80 | 0.96 | 1.43 | 0.30 | 0.32 |
| 1.29 | 0.48 | 1.12 | 2.02 | 0.62 |
| 0.95 | 0.92 | 0.85 | 0.89 | 2.62 |
| 1.22 | 1.40 | 1.14 | 1.56 | 0.53 |
| 0.88 | 1.23 | 0.98 | 1.09 | 0.42 |
| 0.97 | 0.79 | 1.28 | 1.42 | 1.01 |
| 1.01 | 1.03 | 1.52 | 1.11 | 1.68 |
| 0.53 | 0.41 | 0.37 | 1.13 | 1.48 |
| 1.24 | 0.84 | 1.35 | 0.65 | 0.73 |
| 0.98 | 1.10 | 1.47 | 1.38 | 0.55 |
| 0.91 | 0.70 | 1.29 | 1.29 | 1.06 |
| 1.07 | 0.96 | 1.61 | 1.38 | 0.43 |
| 0.94 | 0.83 | 1.33 | 1.56 | 1.07 |
| 1.30 | 1.38 | 1.12 | 0.74 | 16.67 |
| 0.73 | 0.69 | 0.42 | 1.06 | 3.04 |
| 0.72 | 0.43 | 0.89 | 0.91 | 7.84 |
| 1.32 | 0.95 | 1.04 | 1.91 | 17.41 |
| 0.74 | 1.80 | 1.03 | 0.83 | 0.53 |
| 1.08 | 1.10 | 1.09 | 1.08 | 1.09 |
| 0.94 | 1.06 | 1.20 | 1.08 | 0.57 |
| 0.92 | 0.98 | 0.99 | 0.97 | 0.43 |
| 1.03 | 1.18 | 1.05 | 0.82 | 1.14 |
| 1.10 | 1.57 | 1.25 | 1.08 | 0.74 |
| 0.89 | 0.70 | 1.07 | 0.93 | 1.01 |
| 0.65 | 0.53 | 0.46 | 0.92 | 0.68 |
| 1.12 | 0.83 | 0.98 | 0.89 | 1.29 |
| 0.80 | 0.89 | 0.86 | 0.68 | 5.84 |
| 0.95 | 1.10 | 1.00 | 1.20 | 0.68 |
| 1.14 | 1.04 | 1.88 | 1.26 | 0.64 |
| 1.07 | 1.10 | 1.12 | 0.96 | 0.97 |
| 0.83 | 1.01 | 0.90 | 0.90 | 0.85 |
| 0.85 | 0.94 | 1.00 | 1.02 | 0.85 |
| 1.43 | 1.66 | 3.05 | 3.40 | 0.99 |
| 1.23 | 1.82 | 1.49 | 1.79 | 2.24 |
| 1.12 | 1.55 | 2.27 | 1.21 | 0.71 |
| 0.83 | 1.00 | 0.75 | 1.17 | 1.23 |
| 0.98 | 0.81 | 0.93 | 1.00 | 1.73 |
| 0.91 | 0.83 | 1.14 | 0.99 | 1.02 |
| 1.20 | 2.94 | 1.87 | 0.77 | 1.31 |
| 1.25 | 3.10 | 1.13 | 0.85 | 1.05 |
| 0.91 | 1.14 | 1.07 | 40.93 | 55.13 |
| 0.73 | 0.93 | 1.08 | 0.87 | 1.00 |
| 1.22 | 1.11 | 1.37 | 29.57 | 171.90 |
| 1.00 | 1.00 | 1.00 | 1.36 | 2.24 |
| 1.00 | 1.00 | 1.00 | 1.00 | 3.82 |
| 1.00 | 1.00 | 1.00 | 1.00 | 2.31 |
| 1.07 | 1.50 | 2.50 | 0.86 | 0.55 |
| 1.41 | 1.22 | 1.29 | 0.97 | 1.70 |
| 2.88 | 1.38 | 1.59 | 0.77 | 4.62 |
| 1.26 | 0.61 | 0.52 | 1.26 | 1.14 |
| 0.81 | 1.09 | 1.17 | 1.15 | 1.96 |
| 1.68 | 1.62 | 1.97 | 1.19 | 1.23 |
| 0.87 | 0.99 | 0.93 | 1.02 | 0.91 |
| 0.85 | 0.82 | 0.60 | 0.89 | 0.99 |
| 0.81 | 0.78 | 1.08 | 1.14 | 3.58 |
| 0.81 | 1.00 | 0.92 | 0.74 | 2.08 |
| 0.58 | 0.52 | 0.44 | 0.83 | 1.36 |
| 1.59 | 1.39 | 1.12 | 0.91 | 2.63 |
| 0.74 | 0.87 | 0.87 | 0.83 | 0.45 |
| 0.74 | 1.08 | 1.28 | 0.76 | 1.36 |
| 1.06 | 1.13 | 0.96 | 1.05 | 1.01 |
| 0.69 | 0.73 | 0.92 | 0.93 | 1.13 |
| 0.85 | 1.28 | 0.98 | 1.04 | 0.94 |
| 1.23 | 1.09 | 1.75 | 1.43 | 1.13 |
| 0.86 | 0.92 | 0.91 | 0.99 | 0.89 |
| 0.97 | 0.84 | 0.81 | 0.92 | 0.63 |
| 0.93 | 0.79 | 0.80 | 0.96 | 1.05 |
| 1.24 | 1.00 | 1.00 | 1.03 | 0.96 |
| 0.78 | 1.08 | 1.12 | 1.07 | 0.51 |
| 0.79 | 1.03 | 1.28 | 1.13 | 0.53 |
| 1.30 | 1.26 | 1.77 | 1.72 | 1.00 |
| 0.61 | 0.96 | 0.81 | 0.94 | 0.58 |
| 1.27 | 0.89 | 0.98 | 1.09 | 1.22 |
| 1.30 | 0.91 | 0.91 | 1.54 | 1.39 |
| 0.88 | 0.41 | 1.00 | 0.59 | 0.89 |
| 1.70 | 1.70 | 0.96 | 2.10 | 1.46 |
| 1.02 | 1.45 | 0.74 | 1.67 | 1.32 |
| 0.60 | 0.28 | 0.82 | 0.39 | 0.74 |
| 1.14 | 1.36 | 0.83 | 1.50 | 1.07 |
| 1.21 | 1.06 | 1.64 | 1.57 | 0.90 |
| 0.75 | 0.84 | 0.72 | 0.64 | 0.89 |
| 1.00 | 0.99 | 1.02 | 1.14 | 0.99 |
| 0.89 | 0.97 | 0.98 | 1.00 | 0.67 |
| 1.03 | 1.05 | 1.00 | 1.10 | 1.05 |
| 0.78 | 0.90 | 1.05 | 1.02 | 0.89 |
| 0.94 | 1.09 | 1.03 | 1.24 | 1.39 |
| 1.12 | 1.62 | 1.32 | 1.70 | 0.82 |
| 1.11 | 0.80 | 0.90 | 0.99 | 1.45 |
| 1.24 | 0.63 | 0.68 | 0.80 | 0.80 |
| 0.93 | 0.81 | 0.86 | 0.98 | 1.39 |
| 1.07 | 0.77 | 0.79 | 0.75 | 0.73 |
| 1.00 | 0.86 | 0.87 | 0.81 | 0.98 |
| 0.73 | 1.16 | 0.95 | 1.07 | 0.54 |
| 1.22 | 1.11 | 1.38 | 1.22 | 1.38 |
| 0.87 | 1.10 | 1.14 | 0.93 | 0.95 |
| 0.80 | 1.18 | 1.02 | 0.90 | 0.80 |
| 0.86 | 1.18 | 1.14 | 1.09 | 1.00 |
| 1.18 | 1.81 | 1.36 | 1.47 | 0.49 |
| 0.94 | 1.57 | 1.10 | 1.15 | 0.46 |
| 0.34 | 1.01 | 0.68 | 1.27 | 0.31 |
| 0.38 | 1.30 | 0.61 | 0.81 | 0.55 |
| 0.83 | 0.85 | 0.89 | 0.83 | 1.07 |
| 1.00 | 1.06 | 1.01 | 1.26 | 1.11 |
| 1.42 | 0.58 | 0.72 | 0.71 | 3.60 |
| 1.12 | 1.16 | 1.20 | 1.12 | 1.19 |
| 1.21 | 1.53 | 1.28 | 1.17 | 1.08 |
| 0.79 | 0.94 | 1.46 | 1.01 | 0.49 |
| 1.31 | 1.18 | 2.41 | 1.51 | 0.97 |
| 1.15 | 1.09 | 1.63 | 1.31 | 0.92 |
| 1.09 | 0.76 | 0.92 | 1.19 | 1.37 |
| 1.08 | 1.38 | 1.17 | 1.18 | 0.42 |
| 1.05 | 1.12 | 1.30 | 1.22 | 0.93 |
| 1.10 | 1.34 | 0.94 | 1.07 | 0.54 |
| 1.18 | 1.28 | 1.27 | 1.08 | 1.06 |
| 0.94 | 1.27 | 1.15 | 1.32 | 0.77 |
| 0.81 | 1.01 | 0.99 | 0.82 | 0.16 |
| 0.96 | 0.87 | 0.97 | 0.93 | 1.34 |
| 0.70 | 0.89 | 1.02 | 1.03 | 1.00 |
| 0.79 | 0.61 | 0.85 | 0.82 | 1.40 |
| 1.27 | 1.16 | 1.24 | 1.14 | 1.09 |
| 0.75 | 0.95 | 0.92 | 0.98 | 0.76 |
| 1.01 | 0.95 | 1.00 | 1.06 | 0.90 |
| 1.06 | 1.04 | 0.94 | 1.21 | 0.78 |
| 0.98 | 0.77 | 1.05 | 0.93 | 2.05 |
| 0.99 | 1.10 | 1.03 | 1.25 | 2.08 |
| 0.84 | 0.72 | 0.74 | 0.89 | 1.24 |
| 0.97 | 0.92 | 0.89 | 0.81 | 0.97 |
| 0.91 | 0.71 | 0.83 | 0.72 | 1.16 |
| 0.97 | 0.95 | 1.00 | 0.83 | 0.68 |
| 0.68 | 0.69 | 0.76 | 0.88 | 0.86 |
| 0.65 | 1.11 | 1.32 | 0.82 | 1.29 |
| 0.70 | 0.70 | 0.78 | 0.86 | 1.23 |
| 0.81 | 0.85 | 0.91 | 0.84 | 1.63 |
| 0.92 | 1.14 | 1.12 | 0.97 | 1.38 |
| 1.16 | 1.19 | 1.15 | 0.97 | 1.08 |
| 0.75 | 0.91 | 0.98 | 0.90 | 0.61 |
| 0.87 | 0.86 | 0.97 | 1.01 | 1.02 |
| 1.28 | 1.16 | 1.24 | 1.22 | 1.24 |
| 0.84 | 1.12 | 1.08 | 0.98 | 1.16 |
| 0.96 | 0.82 | 0.92 | 0.89 | 1.85 |
| 0.93 | 1.05 | 1.18 | 1.02 | 2.57 |
| 0.81 | 0.74 | 0.92 | 0.85 | 1.22 |
| 0.64 | 0.82 | 0.86 | 0.91 | 0.79 |
| 0.78 | 0.83 | 0.99 | 0.84 | 0.49 |
| 0.74 | 0.79 | 0.87 | 0.95 | 1.19 |
| 0.82 | 0.60 | 0.59 | 1.13 | 1.25 |
| 1.02 | 1.06 | 0.99 | 1.14 | 1.29 |
| 1.03 | 1.38 | 1.15 | 1.20 | 1.39 |
| 0.79 | 1.20 | 1.05 | 0.98 | 2.53 |
| 0.78 | 0.81 | 0.67 | 0.99 | 1.18 |
| 0.88 | 1.04 | 1.20 | 1.53 | 1.22 |
| 1.14 | 1.25 | 1.25 | 1.34 | 1.39 |
| 0.89 | 1.20 | 1.18 | 1.47 | 0.79 |
| 0.75 | 0.64 | 0.65 | 0.90 | 0.57 |
| 0.92 | 0.97 | 0.99 | 0.83 | 1.16 |
| 0.90 | 1.10 | 1.10 | 1.00 | 1.61 |
| 0.87 | 0.91 | 0.86 | 1.11 | 0.85 |
| 0.90 | 0.75 | 0.75 | 1.86 | 3.71 |
| 0.79 | 1.14 | 1.27 | 1.46 | 0.70 |
| 0.74 | 0.80 | 0.76 | 0.81 | 1.04 |
| 0.65 | 0.71 | 0.69 | 0.92 | 1.39 |
| 0.87 | 0.77 | 0.77 | 0.74 | 1.12 |
| 0.61 | 0.97 | 0.88 | 0.85 | 0.71 |
| 0.64 | 1.18 | 1.42 | 1.42 | 0.77 |
| 1.26 | 0.74 | 0.78 | 0.69 | 1.49 |
| 0.62 | 1.01 | 0.87 | 1.11 | 0.52 |
| 0.75 | 1.23 | 1.08 | 1.29 | 0.60 |
| 1.10 | 1.24 | 1.35 | 1.36 | 0.76 |
| 0.94 | 1.18 | 1.10 | 0.96 | 3.02 |
| 1.04 | 1.32 | 1.17 | 1.00 | 5.89 |
| 1.03 | 1.19 | 1.39 | 1.25 | 0.70 |
| 0.91 | 0.92 | 0.87 | 0.88 | 0.84 |
| 1.17 | 1.35 | 0.89 | 1.33 | 0.89 |
| 1.03 | 0.99 | 1.04 | 0.86 | 0.86 |
| 1.12 | 1.03 | 1.07 | 0.97 | 1.03 |
| 0.89 | 0.99 | 0.89 | 0.91 | 0.82 |
| 0.66 | 0.74 | 0.86 | 0.60 | 0.59 |
| 1.14 | 1.03 | 0.84 | 1.37 | 0.63 |
| 0.95 | 1.07 | 0.93 | 1.27 | 0.93 |
| 0.77 | 0.94 | 0.93 | 1.00 | 0.64 |
| 1.38 | 2.45 | 1.86 | 0.89 | 1.35 |
| 1.00 | 1.14 | 1.18 | 0.93 | 1.12 |
| 0.91 | 0.92 | 1.07 | 0.92 | 0.88 |
| 0.96 | 1.07 | 1.03 | 0.96 | 0.65 |
| 1.13 | 0.69 | 1.16 | 0.92 | 0.50 |
| 0.92 | 1.04 | 0.93 | 0.88 | 1.21 |
| 1.12 | 1.43 | 1.30 | 1.49 | 0.48 |
| 1.04 | 1.12 | 1.03 | 0.91 | 1.53 |
| 1.10 | 1.15 | 1.08 | 1.30 | 0.97 |
| 1.35 | 1.55 | 2.64 | 1.82 | 0.98 |
| 0.84 | 1.19 | 2.18 | 0.88 | 1.14 |
| 1.14 | 1.22 | 2.12 | 1.72 | 0.94 |
| 0.94 | 1.43 | 2.44 | 1.19 | 1.27 |
| 1.00 | 1.43 | 2.64 | 1.35 | 1.14 |
| 0.67 | 1.43 | 2.50 | 1.16 | 1.55 |
| 1.45 | 1.34 | 1.41 | 1.35 | 1.10 |
| 0.94 | 1.08 | 0.87 | 1.24 | 0.97 |
| 0.94 | 1.13 | 0.97 | 1.20 | 0.91 |
| 1.30 | 0.98 | 0.92 | 1.82 | 0.57 |
| 1.21 | 1.20 | 1.13 | 1.34 | 0.65 |
| 1.02 | 1.60 | 1.03 | 1.17 | 0.80 |
| 0.92 | 1.62 | 0.94 | 1.34 | 0.36 |
| 0.97 | 0.99 | 1.75 | 2.11 | 0.97 |
| 0.70 | 1.14 | 0.85 | 1.09 | 0.41 |
| 1.28 | 1.22 | 1.46 | 1.05 | 7.20 |
| 0.80 | 0.69 | 1.03 | 0.64 | 1.74 |
| 0.92 | 0.97 | 1.01 | 1.07 | 0.81 |
| 1.05 | 1.00 | 1.53 | 1.58 | 1.28 |
| 0.82 | 0.67 | 1.10 | 1.34 | 0.96 |
| 0.98 | 0.98 | 1.57 | 1.40 | 1.13 |
| 1.38 | 1.21 | 1.45 | 1.10 | 13.88 |
| 0.80 | 1.22 | 1.21 | 1.14 | 0.47 |
| 0.89 | 0.84 | 1.06 | 0.81 | 0.73 |
| 1.20 | 0.94 | 0.75 | 1.28 | 1.49 |
| 0.93 | 0.87 | 0.80 | 0.84 | 0.94 |
| 0.97 | 0.85 | 0.82 | 0.98 | 0.92 |
| 1.24 | 1.09 | 1.62 | 1.42 | 0.93 |
| 0.87 | 0.90 | 0.83 | 0.63 | 1.40 |
| 1.79 | 1.13 | 1.95 | 0.88 | 4.39 |
| 0.88 | 0.97 | 0.99 | 1.13 | 1.02 |
| 0.95 | 0.96 | 0.88 | 1.27 | 1.10 |
| 1.09 | 1.00 | 0.98 | 1.17 | 1.08 |
| 0.99 | 1.01 | 1.08 | 1.01 | 1.32 |
| 0.99 | 0.95 | 1.32 | 0.94 | 1.43 |
| 142.38 | 0.88 | 115.91 | 0.75 | 72.80 |
| 53.54 | 2.52 | 75.15 | 0.62 | 56.31 |
| 0.85 | 1.24 | 0.96 | 0.84 | 0.84 |
| 0.83 | 1.11 | 1.03 | 1.15 | 0.67 |
| 0.79 | 1.06 | 1.84 | 1.13 | 0.50 |
| 0.73 | 0.61 | 0.88 | 0.55 | 0.72 |
| 1.30 | 1.07 | 2.11 | 1.65 | 0.92 |
| 0.87 | 0.88 | 0.92 | 0.94 | 1.04 |
| 0.93 | 0.95 | 1.00 | 1.04 | 0.97 |
| 1.00 | 1.08 | 0.96 | 1.09 | 1.02 |
| 0.80 | 0.90 | 1.00 | 1.08 | 0.94 |
| 1.07 | 0.87 | 0.84 | 0.90 | 0.77 |
| 0.92 | 0.81 | 0.71 | 1.14 | 0.89 |
| 0.75 | 0.63 | 0.94 | 0.56 | 0.48 |
| 1.12 | 1.07 | 1.26 | 1.22 | 0.98 |
| 0.94 | 0.92 | 0.80 | 0.70 | 1.20 |
| 1.03 | 1.25 | 1.26 | 0.87 | 0.97 |
| 1.21 | 1.18 | 2.01 | 1.28 | 0.99 |
| 0.85 | 1.15 | 1.14 | 1.22 | 0.70 |
| 1.11 | 1.13 | 1.62 | 1.51 | 1.30 |
| 0.78 | 0.87 | 0.82 | 0.94 | 0.76 |
| 0.89 | 1.06 | 1.07 | 1.03 | 0.69 |
| 0.78 | 0.77 | 0.96 | 1.14 | 0.84 |
| 0.86 | 0.86 | 0.86 | 0.86 | 37.21 |
| 0.54 | 0.54 | 0.70 | 0.76 | 73.48 |
| 2.16 | 1.99 | 2.98 | 0.64 | 4.25 |
| 8.86 | 3.82 | 11.61 | 0.87 | 5.99 |
| 2.64 | 3.91 | 1.99 | 0.97 | 2.74 |
| 1.98 | 2.90 | 1.30 | 0.98 | 2.05 |
| 2.91 | 1.87 | 1.58 | 0.53 | 3.01 |
| 0.91 | 1.03 | 0.98 | 1.03 | 0.87 |
| 0.84 | 0.70 | 0.56 | 0.60 | 0.47 |
| 0.71 | 0.78 | 0.89 | 0.80 | 1.40 |
| 1.52 | 0.55 | 0.54 | 8.20 | 65.21 |
| 0.76 | 1.25 | 1.13 | 0.98 | 1.06 |
| 1.08 | 1.07 | 1.15 | 1.71 | 1.17 |
| 1.45 | 1.27 | 1.60 | 2.90 | 0.36 |
| 1.08 | 1.22 | 1.34 | 1.35 | 0.99 |
| 0.68 | 1.07 | 1.18 | 0.71 | 0.69 |
| 0.85 | 0.98 | 1.02 | 1.13 | 0.68 |
| 1.04 | 1.01 | 1.67 | 1.13 | 0.64 |
| 1.36 | 1.32 | 1.26 | 1.22 | 0.58 |
| 1.03 | 1.27 | 1.00 | 1.36 | 1.65 |
| 0.91 | 1.02 | 0.88 | 0.99 | 0.61 |
| 0.93 | 1.03 | 1.05 | 1.08 | 1.08 |
| 0.90 | 0.96 | 0.91 | 0.84 | 1.05 |
| 0.98 | 0.93 | 1.19 | 0.81 | 1.59 |
| 0.61 | 0.57 | 0.59 | 0.94 | 0.97 |
| 1.23 | 1.61 | 2.72 | 1.40 | 1.12 |
| 1.43 | 1.69 | 3.24 | 3.15 | 1.76 |
| 1.44 | 1.71 | 3.07 | 3.13 | 1.70 |
| 1.36 | 1.48 | 3.08 | 2.55 | 1.29 |
| 1.40 | 1.48 | 3.00 | 2.52 | 1.30 |
| 1.06 | 1.15 | 1.40 | 0.99 | 0.97 |
| 1.18 | 1.10 | 0.78 | 1.18 | 0.85 |
| 0.79 | 0.95 | 1.00 | 0.71 | 0.89 |
| 0.75 | 1.09 | 1.13 | 0.98 | 0.94 |
| 0.91 | 0.88 | 0.96 | 0.91 | 0.86 |
| 0.97 | 1.31 | 1.11 | 1.08 | 1.91 |
| 0.98 | 1.30 | 0.99 | 0.85 | 1.32 |
| 0.81 | 1.02 | 0.93 | 1.00 | 0.68 |
| 1.05 | 1.36 | 1.69 | 1.12 | 2.12 |
| 1.06 | 1.16 | 1.36 | 1.36 | 1.07 |
| 0.91 | 1.00 | 0.89 | 0.90 | 1.45 |
| 0.94 | 0.93 | 0.98 | 1.04 | 0.91 |
| 0.98 | 1.17 | 1.24 | 1.39 | 0.91 |
| 0.84 | 0.86 | 0.86 | 0.83 | 0.76 |
| 0.87 | 1.48 | 1.46 | 0.56 | 0.36 |
| 0.88 | 0.99 | 1.01 | 1.10 | 1.13 |
| 0.95 | 1.09 | 1.49 | 1.17 | 1.76 |
| 0.48 | 0.27 | 0.41 | 0.63 | 0.92 |
| 1.05 | 1.29 | 1.07 | 1.27 | 0.83 |
| 1.24 | 1.08 | 1.27 | 0.98 | 1.66 |
| 1.06 | 0.58 | 1.91 | 0.43 | 1.05 |
| 0.76 | 0.43 | 0.72 | 0.52 | 1.85 |
| 0.73 | 1.03 | 1.19 | 0.65 | 1.52 |
| 0.71 | 0.71 | 0.76 | 0.87 | 0.73 |
| 1.11 | 1.12 | 1.97 | 1.39 | 0.77 |
| 0.97 | 1.17 | 0.94 | 1.36 | 0.83 |
| 1.24 | 1.45 | 1.56 | 2.67 | 0.24 |
| 1.05 | 0.98 | 1.09 | 1.04 | 0.95 |
| 0.91 | 0.82 | 0.95 | 0.72 | 0.31 |
| 1.24 | 1.06 | 0.98 | 1.20 | 0.62 |
| 1.56 | 1.10 | 1.70 | 1.16 | 0.89 |
| 1.56 | 1.22 | 1.18 | 1.32 | 1.33 |
| 1.03 | 0.95 | 0.94 | 0.84 | 0.71 |
| 0.84 | 0.94 | 0.89 | 0.86 | 0.77 |
| 0.81 | 0.85 | 1.05 | 0.85 | 0.69 |
| 0.93 | 0.89 | 0.75 | 0.86 | 0.89 |
| 1.21 | 0.85 | 1.07 | 0.98 | 1.14 |
| 0.87 | 0.52 | 0.79 | 0.59 | 1.05 |
| 0.89 | 0.91 | 0.90 | 0.95 | 0.81 |
| 0.84 | 0.85 | 0.95 | 0.88 | 1.08 |
| 0.97 | 1.01 | 0.98 | 1.17 | 0.90 |
| 0.92 | 0.77 | 0.95 | 0.89 | 0.91 |
| 0.92 | 0.73 | 0.90 | 0.92 | 1.02 |
| 1.04 | 1.01 | 1.03 | 1.24 | 0.89 |
| 0.91 | 0.71 | 0.77 | 1.07 | 0.92 |
| 0.98 | 0.84 | 0.87 | 1.18 | 0.95 |
| 1.15 | 1.14 | 1.00 | 1.41 | 0.91 |
| 1.35 | 1.55 | 0.91 | 1.52 | 1.29 |
| 1.16 | 1.16 | 1.14 | 1.11 | 0.97 |
| 0.89 | 0.88 | 0.86 | 0.90 | 0.84 |
| 1.00 | 1.01 | 0.91 | 1.12 | 0.99 |
| 1.13 | 1.04 | 1.11 | 1.01 | 0.84 |
| 1.06 | 0.87 | 0.93 | 1.21 | 0.81 |
| 0.85 | 0.80 | 0.88 | 0.82 | 0.74 |
| 0.97 | 0.97 | 0.99 | 0.98 | 0.77 |
| 1.01 | 1.04 | 0.99 | 1.01 | 0.91 |
| 1.05 | 1.16 | 1.04 | 1.18 | 0.93 |
| 1.27 | 1.12 | 1.12 | 1.21 | 1.19 |
| 1.06 | 1.03 | 0.89 | 0.95 | 0.72 |
| 1.07 | 1.25 | 1.21 | 1.01 | 1.26 |
| 0.78 | 1.05 | 0.93 | 1.09 | 0.65 |
| 0.87 | 0.91 | 1.24 | 1.14 | 0.60 |
| 0.95 | 0.88 | 0.99 | 1.11 | 0.80 |
| 0.99 | 1.02 | 1.03 | 1.18 | 1.04 |
| 1.41 | 1.18 | 1.21 | 1.52 | 1.23 |
| 0.99 | 0.77 | 0.81 | 0.67 | 1.10 |
| 1.13 | 1.09 | 1.92 | 1.36 | 0.70 |
| 1.58 | 1.10 | 1.97 | 1.30 | 1.55 |
| 0.95 | 0.83 | 0.77 | 0.92 | 0.98 |
| 0.73 | 0.42 | 0.68 | 0.59 | 0.75 |
| 1.15 | 1.29 | 1.24 | 1.70 | 0.38 |
| 0.92 | 0.99 | 0.98 | 1.02 | 0.96 |
| 0.84 | 0.96 | 0.87 | 0.69 | 0.68 |
| 0.98 | 1.02 | 0.92 | 0.91 | 0.85 |
| 1.02 | 1.04 | 1.19 | 1.18 | 1.09 |
| 0.32 | 0.35 | 6.75 | 0.61 | 0.24 |
| 0.45 | 0.64 | 4.70 | 0.93 | 0.73 |
| 0.33 | 0.47 | 2.23 | 0.61 | 0.45 |
| 1.03 | 0.72 | 1.24 | 0.94 | 1.08 |
| 1.11 | 3.25 | 0.71 | 1.09 | 367.34 |
| 0.66 | 0.66 | 2.11 | 0.67 | 5.04 |
| 2.90 | 7.50 | 3.30 | 0.87 | 0.89 |
| 0.63 | 0.70 | 12.94 | 1.05 | 0.56 |
| 1.07 | 1.09 | 1.71 | 1.66 | 0.75 |
| 1.14 | 0.96 | 1.87 | 1.21 | 0.51 |
| 1.03 | 0.98 | 1.52 | 1.34 | 0.89 |
| 0.81 | 0.95 | 0.75 | 1.12 | 0.98 |
| 0.72 | 0.77 | 0.67 | 0.77 | 1.02 |
| 1.10 | 0.97 | 1.12 | 0.99 | 1.10 |
| 0.98 | 0.99 | 1.06 | 0.96 | 1.48 |
| 0.95 | 1.02 | 1.04 | 1.07 | 1.07 |
| 1.11 | 0.86 | 1.09 | 0.99 | 1.25 |
| 1.11 | 1.66 | 1.24 | 1.80 | 1.23 |
| 0.73 | 1.10 | 1.11 | 0.88 | 0.65 |
| 0.92 | 0.74 | 0.65 | 1.30 | 0.78 |
| 0.95 | 1.06 | 1.37 | 1.09 | 1.06 |
| 1.13 | 0.76 | 0.67 | 1.48 | 2.66 |
| 0.79 | 0.68 | 0.88 | 1.28 | 0.92 |
| 0.99 | 1.11 | 1.06 | 1.22 | 0.94 |
| 0.86 | 1.34 | 1.15 | 1.18 | 0.77 |
| 0.75 | 0.53 | 0.92 | 0.54 | 0.84 |
| 1.25 | 1.17 | 2.26 | 1.45 | 0.81 |
| 0.92 | 1.08 | 1.04 | 1.07 | 1.12 |
| 2.53 | 0.56 | 1.25 | 1.31 | 96.73 |
| 0.84 | 0.96 | 0.93 | 0.94 | 0.83 |
| 0.87 | 0.92 | 0.92 | 0.97 | 0.90 |
| 1.20 | 1.15 | 2.02 | 1.40 | 0.82 |
| 1.18 | 1.23 | 1.89 | 1.36 | 0.98 |
| 1.09 | 0.80 | 1.81 | 0.87 | 1.10 |
| 1.15 | 1.08 | 0.97 | 1.33 | 1.26 |
| 1.03 | 0.85 | 0.86 | 0.93 | 0.92 |
| 0.83 | 1.04 | 1.07 | 1.07 | 0.97 |
| 0.89 | 0.72 | 0.78 | 0.99 | 0.95 |
| 1.21 | 0.43 | 0.96 | 2.60 | 0.58 |
| 11.99 | 14.60 | 45.21 | 82.94 | 0.85 |
| 1.04 | 0.87 | 1.55 | 1.32 | 4.82 |
| 1.21 | 1.08 | 1.39 | 0.76 | 4.00 |
| 0.55 | 0.37 | 0.43 | 0.84 | 1.61 |
| 0.80 | 0.87 | 0.86 | 0.93 | 0.85 |
| 1.27 | 1.04 | 1.01 | 0.93 | 1.36 |
| 0.98 | 0.66 | 0.79 | 0.86 | 1.05 |
| 1.12 | 1.63 | 1.55 | 1.06 | 0.62 |
| 1.00 | 0.81 | 0.83 | 0.92 | 0.88 |
| 0.64 | 0.66 | 0.79 | 0.94 | 0.84 |

Statistical Values (p-value is t-test statistic, q-value is false discovery statistic)
Welch's Two-Sample t-Test (comparison between noted conditions)

| MOL361 | | 1E2 | | H121 | | T129 | | CONV | |
|---|---|---|---|---|---|---|---|---|---|
| GF | | GF | | GF | | GF | | GF | |
| p-value | q-value | p-value | q-value | p-value | q-value | p-value | q-value | p-value | q-value |
| 0.0158 | 0.2237 | 0.6126 | 0.7739 | 0.1165 | 0.2991 | 0.4947 | 0.7056 | 0.0071 | 0.0192 |
| 0.0272 | 0.2533 | 0.6074 | 0.7706 | 0.8195 | 0.6467 | 0.4013 | 0.6735 | 0.0039 | 0.0141 |
| 0.7797 | 0.6954 | 0.8165 | 0.8179 | 0.0875 | 0.2629 | 0.5929 | 0.7490 | 0.0084 | 0.0216 |
| 0.9376 | 0.7324 | 0.5875 | 0.7627 | 0.1569 | 0.3276 | 0.6290 | 0.7630 | 0.7148 | 0.4158 |
| 0.2310 | 0.4709 | 0.0894 | 0.4917 | 0.3076 | 0.4471 | 0.4305 | 0.6821 | 0.0040 | 0.0141 |
| 0.1219 | 0.3802 | 0.3461 | 0.6614 | 0.0389 | 0.1920 | 0.9316 | 0.8246 | 0.0193 | 0.0395 |
| 0.3166 | 0.5349 | 0.1210 | 0.5031 | 0.0992 | 0.2799 | 0.8608 | 0.8201 | 0.4795 | 0.3275 |
| 0.4588 | 0.6004 | 0.3055 | 0.6550 | 0.1398 | 0.3151 | 0.5718 | 0.7389 | 0.2544 | 0.2190 |
| 0.4772 | 0.6122 | 0.0032 | 0.1565 | 0.0477 | 0.2104 | 0.3150 | 0.6635 | 0.1582 | 0.1645 |
| 0.0264 | 0.2518 | 0.7987 | 0.8177 | 0.5191 | 0.5510 | 0.8096 | 0.8090 | 0.0290 | 0.0511 |
| 0.6267 | 0.6579 | 0.0090 | 0.2084 | 0.1657 | 0.3370 | 0.0496 | 0.5116 | 0.0046 | 0.0148 |
| 0.6525 | 0.6642 | 0.0621 | 0.4455 | 0.0020 | 0.0487 | 0.1017 | 0.5714 | 0.0800 | 0.1009 |
| 0.0639 | 0.3199 | 0.0018 | 0.1565 | 0.0435 | 0.2004 | 0.0525 | 0.5116 | 0.0057 | 0.0168 |
| 0.0278 | 0.2533 | 0.4968 | 0.7261 | 0.0384 | 0.1918 | 0.9522 | 0.8303 | 0.8798 | 0.4643 |
| 0.5818 | 0.6459 | 0.8386 | 0.8280 | 0.6043 | 0.5870 | 0.8558 | 0.8188 | 0.4068 | 0.2963 |
| 0.3231 | 0.5407 | 0.1916 | 0.5973 | 0.1625 | 0.3342 | 0.5224 | 0.7144 | 0.0627 | 0.0847 |
| 0.6829 | 0.6642 | 0.0232 | 0.2968 | 0.0029 | 0.0549 | 0.2306 | 0.6375 | 0.1066 | 0.1233 |
| 0.9273 | 0.7294 | 0.0403 | 0.3763 | 0.1467 | 0.3169 | 0.9112 | 0.8246 | 0.0045 | 0.0147 |
| 0.1779 | 0.4322 | 0.0090 | 0.2084 | 0.0012 | 0.0375 | 0.0665 | 0.5225 | 0.0010 | 0.0057 |
| 0.3162 | 0.5349 | 0.0730 | 0.4701 | 0.1637 | 0.3342 | 0.5423 | 0.7285 | 0.9945 | 0.4951 |
| 0.5426 | 0.6273 | 0.1757 | 0.5741 | 0.3826 | 0.4833 | 0.4141 | 0.6821 | 0.0015 | 0.0077 |
| 0.3444 | 0.5504 | 0.7411 | 0.8059 | 0.7265 | 0.6254 | 0.3764 | 0.6648 | 0.0000 | 0.0007 |
| 0.9473 | 0.7353 | 0.8362 | 0.8269 | 0.4738 | 0.5264 | 0.8531 | 0.8177 | 0.0001 | 0.0010 |
| 0.0063 | 0.2237 | 0.3214 | 0.6550 | 0.0045 | 0.0637 | 0.0104 | 0.3368 | 0.0032 | 0.0126 |
| 0.2292 | 0.4709 | 0.3835 | 0.6811 | 0.5071 | 0.5459 | 0.0720 | 0.5234 | 0.0383 | 0.0612 |
| 0.3821 | 0.5570 | 0.3709 | 0.6705 | 0.5163 | 0.5510 | 0.1483 | 0.6116 | 0.0015 | 0.0076 |
| 0.5004 | 0.6198 | 0.7096 | 0.8004 | 0.1811 | 0.3581 | 0.2329 | 0.6380 | 0.0382 | 0.0612 |
| 0.0079 | 0.2237 | 0.5845 | 0.7622 | 0.0631 | 0.2352 | 0.6087 | 0.7535 | 0.0658 | 0.0879 |
| 0.2207 | 0.4709 | 0.2246 | 0.6090 | 0.5368 | 0.5578 | 0.6136 | 0.7548 | 0.0386 | 0.0615 |
| 0.2220 | 0.4709 | 0.2841 | 0.6498 | 0.0386 | 0.1918 | 0.6287 | 0.7630 | 0.0035 | 0.0133 |
| 0.0093 | 0.2237 | 0.2008 | 0.5977 | 0.3613 | 0.4796 | 0.3719 | 0.6648 | 0.0521 | 0.0761 |
| 0.0788 | 0.3430 | 0.7990 | 0.8177 | 0.0768 | 0.2530 | 0.5689 | 0.7388 | 0.0382 | 0.0612 |
| 0.1178 | 0.3750 | 0.4445 | 0.7041 | 0.7228 | 0.6254 | 0.9461 | 0.8288 | 0.0333 | 0.0562 |
| 0.1848 | 0.4361 | 0.9684 | 0.8488 | 0.5925 | 0.5837 | 0.1143 | 0.5856 | 0.0770 | 0.0976 |
| 0.0806 | 0.3442 | 0.4329 | 0.7007 | 0.7726 | 0.6352 | 0.4560 | 0.6890 | 0.0018 | 0.0089 |
| 0.0252 | 0.2518 | 0.8018 | 0.8177 | 0.6275 | 0.5966 | 0.5779 | 0.7402 | 0.0028 | 0.0114 |
| 0.0308 | 0.2635 | 0.2939 | 0.6533 | 0.1113 | 0.2946 | 0.8152 | 0.8113 | 0.0002 | 0.0020 |
| 0.0159 | 0.2237 | 0.7783 | 0.8174 | 0.3989 | 0.4941 | 0.4999 | 0.7056 | 0.0105 | 0.0258 |
| 0.0023 | 0.1952 | 0.3156 | 0.6550 | 0.0108 | 0.0965 | 0.0216 | 0.3996 | 0.0017 | 0.0086 |
| 0.9827 | 0.7411 | 0.4637 | 0.7101 | 0.7705 | 0.6352 | 0.5723 | 0.7389 | 0.0073 | 0.0194 |
| 0.1711 | 0.4288 | 0.8128 | 0.8177 | 0.2415 | 0.3993 | 0.7053 | 0.7879 | 0.0036 | 0.0136 |
| 0.9179 | 0.7281 | 0.9159 | 0.8444 | 0.5495 | 0.5654 | 0.5115 | 0.7070 | 0.7464 | 0.4272 |
| 0.5739 | 0.6410 | 0.1601 | 0.5502 | 0.6561 | 0.6121 | 0.8155 | 0.8113 | 0.7607 | 0.4320 |
| 0.5666 | 0.6393 | 0.2408 | 0.6241 | 0.8573 | 0.6565 | 0.9676 | 0.8347 | 0.8777 | 0.4643 |
| 0.2731 | 0.5009 | 0.4819 | 0.7227 | 0.2330 | 0.3956 | 0.9977 | 0.8421 | 0.1982 | 0.1884 |
| 0.3374 | 0.5484 | 0.3343 | 0.6550 | 0.5255 | 0.5525 | 0.6978 | 0.7879 | 0.2398 | 0.2129 |
| 0.1443 | 0.4142 | 0.1411 | 0.5147 | 0.2904 | 0.4354 | 0.5006 | 0.7056 | 0.0543 | 0.0771 |
| 0.7569 | 0.6906 | 0.7912 | 0.8177 | 0.9873 | 0.6904 | 0.3752 | 0.6648 | 0.5296 | 0.3439 |
| 0.7763 | 0.6952 | 0.8653 | 0.8352 | 0.9484 | 0.6745 | 0.7940 | 0.8058 | 0.4593 | 0.3224 |
| 0.3122 | 0.5330 | 0.6021 | 0.7706 | 0.8315 | 0.6518 | 0.0406 | 0.5116 | 0.1879 | 0.1828 |
| 0.0183 | 0.2422 | 0.9671 | 0.8487 | 0.0377 | 0.1918 | 0.9226 | 0.8246 | 0.0004 | 0.0031 |
| 0.0225 | 0.2518 | 0.1800 | 0.5796 | 0.7575 | 0.6335 | 0.0685 | 0.5225 | 0.5296 | 0.3439 |
| 0.3764 | 0.5570 | 0.3546 | 0.6631 | 0.9317 | 0.6736 | 0.8696 | 0.8201 | 0.2005 | 0.1899 |
| 0.0687 | 0.3270 | 0.0075 | 0.2084 | 0.0399 | 0.1921 | 0.1801 | 0.6258 | 0.2976 | 0.2403 |
| 0.3667 | 0.5570 | 0.8038 | 0.8177 | 0.2520 | 0.4091 | 0.6113 | 0.7543 | 0.1396 | 0.1499 |
| 0.1844 | 0.4361 | 0.5385 | 0.7501 | 0.6820 | 0.6153 | 0.8342 | 0.8170 | 0.0058 | 0.0169 |
| 0.5201 | 0.6257 | 0.1914 | 0.5973 | 0.8473 | 0.6538 | 0.2587 | 0.6561 | 0.1069 | 0.1233 |
| 0.3560 | 0.5539 | 0.5110 | 0.7340 | 0.8756 | 0.6657 | 0.7367 | 0.7941 | 0.3221 | 0.2516 |
| 0.5025 | 0.6204 | 0.2889 | 0.6500 | 0.6241 | 0.5957 | 0.7009 | 0.7879 | 0.0866 | 0.1047 |
| 0.0081 | 0.2237 | 0.6918 | 0.7890 | 0.0032 | 0.0549 | 0.0033 | 0.2605 | 0.0066 | 0.0181 |
| 0.6664 | 0.6642 | 0.0272 | 0.3156 | 0.1194 | 0.3042 | 0.0168 | 0.3996 | 0.0321 | 0.0545 |
| 0.1341 | 0.4035 | 0.7966 | 0.8177 | 0.7851 | 0.6389 | 0.4268 | 0.6821 | 0.1943 | 0.1869 |
| 0.7982 | 0.7024 | 0.2099 | 0.5977 | 0.3115 | 0.4486 | 0.2034 | 0.6258 | 0.1842 | 0.1811 |
| 0.3210 | 0.5392 | 0.5027 | 0.7316 | 0.8124 | 0.6465 | 0.9257 | 0.8246 | 0.8578 | 0.4604 |
| 0.0644 | 0.3199 | 0.7121 | 0.8004 | 0.0902 | 0.2629 | 0.7448 | 0.7941 | 0.0354 | 0.0594 |
| 0.7256 | 0.6785 | 0.2508 | 0.6323 | 0.6641 | 0.6129 | 0.2005 | 0.6258 | 0.5599 | 0.3561 |
| 0.8583 | 0.7196 | 0.4525 | 0.7097 | 0.0684 | 0.2437 | 0.5631 | 0.7370 | 0.7688 | 0.4346 |
| 0.0302 | 0.2616 | 0.0043 | 0.1828 | 0.0272 | 0.1652 | 0.4454 | 0.6869 | 0.3493 | 0.2670 |
| 0.9594 | 0.7367 | 0.1083 | 0.4999 | 0.3046 | 0.4468 | 0.3674 | 0.6648 | 0.4037 | 0.2951 |
| 0.4413 | 0.5969 | 0.4004 | 0.6946 | 0.9775 | 0.6851 | 0.1991 | 0.6258 | 0.4355 | 0.3094 |
| 0.5719 | 0.6402 | 0.0627 | 0.4455 | 0.1121 | 0.2946 | 0.1746 | 0.6256 | 0.2013 | 0.1902 |
| 0.0023 | 0.1952 | 0.0026 | 0.1565 | 0.0572 | 0.2296 | 0.3511 | 0.6648 | 0.1767 | 0.1765 |
| 0.0203 | 0.2497 | 0.3162 | 0.6550 | 0.7188 | 0.6238 | 0.2374 | 0.6397 | 0.8339 | 0.4532 |
| 0.0209 | 0.2497 | 0.5486 | 0.7501 | 0.0755 | 0.2530 | 0.3918 | 0.6735 | 0.1891 | 0.1833 |
| 0.0294 | 0.2612 | 0.0181 | 0.2862 | 0.0829 | 0.2629 | 0.5782 | 0.7402 | 0.3764 | 0.2801 |
| 0.5775 | 0.6422 | 0.9375 | 0.8447 | 0.9324 | 0.6736 | 0.5343 | 0.7215 | 0.2420 | 0.2136 |
| 0.3278 | 0.5435 | 0.4234 | 0.7007 | 0.4417 | 0.5163 | 0.1165 | 0.5885 | 0.0847 | 0.1042 |
| 0.1104 | 0.3672 | 0.3662 | 0.6705 | 0.3763 | 0.4819 | 0.8840 | 0.8246 | 0.9995 | 0.4964 |
| 0.1405 | 0.4089 | 0.7186 | 0.8004 | 0.9174 | 0.6736 | 0.8199 | 0.8124 | 0.3796 | 0.2811 |
| 0.8801 | 0.7219 | 0.0246 | 0.2968 | 0.5805 | 0.5786 | 0.2977 | 0.6604 | 0.3277 | 0.2545 |
| 0.1124 | 0.3697 | 0.6828 | 0.7859 | 0.1870 | 0.3581 | 0.1341 | 0.6069 | 0.1707 | 0.1720 |
| 0.1676 | 0.4260 | 0.7736 | 0.8159 | 0.1199 | 0.3042 | 0.8954 | 0.8246 | 0.3775 | 0.2805 |
| 0.1071 | 0.3668 | 0.1613 | 0.5502 | 0.0114 | 0.1002 | 0.7390 | 0.7941 | 0.3515 | 0.2677 |
| 0.0001 | 0.0401 | 0.2289 | 0.6110 | 0.0040 | 0.0591 | 0.3224 | 0.6635 | 0.0623 | 0.0847 |
| 0.0100 | 0.2237 | 0.2422 | 0.6241 | 0.0005 | 0.0255 | 0.0114 | 0.3368 | 0.0234 | 0.0444 |
| 0.0532 | 0.3182 | 0.6831 | 0.7859 | 0.7551 | 0.6335 | 0.4273 | 0.6821 | 0.0059 | 0.0171 |
| 0.0236 | 0.2518 | 0.4714 | 0.7161 | 0.4089 | 0.4992 | 0.3891 | 0.6718 | 0.0008 | 0.0053 |
| 0.8545 | 0.7193 | 0.2485 | 0.6323 | 0.2071 | 0.3713 | 0.9296 | 0.8246 | 0.6776 | 0.4034 |
| 0.8757 | 0.7219 | 0.1822 | 0.5796 | 0.7563 | 0.6335 | 0.6793 | 0.7844 | 0.1320 | 0.1442 |
| 0.8314 | 0.7149 | 0.1459 | 0.5270 | 0.0974 | 0.2773 | 0.7071 | 0.7879 | 0.6965 | 0.4104 |
| 0.2923 | 0.5154 | 0.0670 | 0.4509 | 0.3679 | 0.4812 | 0.7798 | 0.8048 | 0.7217 | 0.4176 |
| 0.0068 | 0.2237 | 0.3272 | 0.6550 | 0.1018 | 0.2849 | 0.1066 | 0.5768 | 0.4284 | 0.3054 |
| 0.5601 | 0.6343 | 0.1212 | 0.5031 | 0.4155 | 0.4992 | 0.5258 | 0.7177 | 0.0849 | 0.1042 |
| 0.4616 | 0.6004 | 0.0614 | 0.4455 | 0.3111 | 0.4486 | 0.5423 | 0.7285 | 0.0590 | 0.0819 |
| 0.0003 | 0.0868 | 0.6469 | 0.7782 | 0.5824 | 0.5786 | 0.5513 | 0.7345 | 0.0023 | 0.0104 |
| 0.0382 | 0.2966 | 0.0631 | 0.4455 | 0.0228 | 0.1506 | 0.7928 | 0.8058 | 0.8964 | 0.4668 |
| 0.0111 | 0.2237 | 0.0126 | 0.2491 | 0.0560 | 0.2294 | 0.1574 | 0.6131 | 0.1614 | 0.1670 |
| 0.2076 | 0.4610 | 0.3181 | 0.6550 | 0.3910 | 0.4880 | 0.7163 | 0.7887 | 0.6509 | 0.3926 |
| 0.2011 | 0.4548 | 0.0439 | 0.3767 | 0.0900 | 0.2629 | 0.2144 | 0.6280 | 0.1969 | 0.1881 |
| 0.1681 | 0.4260 | 0.3964 | 0.6946 | 0.1887 | 0.3581 | 0.8712 | 0.8201 | 0.9353 | 0.4787 |
| 0.3480 | 0.5514 | 0.0132 | 0.2491 | 0.0099 | 0.0944 | 0.5733 | 0.7390 | 0.0062 | 0.0176 |
| 0.2511 | 0.4761 | 0.0367 | 0.3731 | 0.0086 | 0.0901 | 0.1577 | 0.6131 | 0.8495 | 0.4589 |
| 0.0098 | 0.2237 | 0.2317 | 0.6142 | 0.0005 | 0.0255 | 0.0027 | 0.2605 | 0.0115 | 0.0278 |
| 0.0096 | 0.2237 | 0.4580 | 0.7101 | 0.3604 | 0.4796 | 0.5103 | 0.7066 | 0.2081 | 0.1938 |
| 0.0288 | 0.2596 | 0.3093 | 0.6550 | 0.4873 | 0.5338 | 0.1975 | 0.6258 | 0.3983 | 0.2916 |
| 0.8728 | 0.7219 | 0.2978 | 0.6533 | 0.8851 | 0.6670 | 0.9474 | 0.8290 | 0.0879 | 0.1054 |
| 0.1943 | 0.4446 | 0.1048 | 0.4999 | 0.0785 | 0.2550 | 0.0271 | 0.4503 | 0.2582 | 0.2194 |
| 0.0489 | 0.3055 | 0.0675 | 0.4509 | 0.2585 | 0.4111 | 0.1456 | 0.6069 | 0.2894 | 0.2353 |
| 0.8286 | 0.7149 | 0.9202 | 0.8444 | 0.8846 | 0.6670 | 0.2090 | 0.6280 | 0.0042 | 0.0141 |
| 0.1510 | 0.4183 | 0.2711 | 0.6469 | 0.1504 | 0.3199 | 0.2325 | 0.6380 | 0.0224 | 0.0436 |
| 0.1062 | 0.3668 | 0.1144 | 0.5031 | 0.2085 | 0.3713 | 0.2030 | 0.6258 | 0.0037 | 0.0137 |
| 0.3289 | 0.5435 | 0.1201 | 0.5031 | 0.3996 | 0.4941 | 0.2766 | 0.6595 | 0.7069 | 0.4130 |
| 0.2293 | 0.4709 | 0.4103 | 0.6997 | 0.0283 | 0.1690 | 0.1733 | 0.6256 | 0.0118 | 0.0283 |
| 0.5174 | 0.6257 | 0.2600 | 0.6342 | 0.0404 | 0.1928 | 0.3198 | 0.6635 | 0.0037 | 0.0137 |
| 0.3988 | 0.5691 | 0.3217 | 0.6550 | 0.3091 | 0.4473 | 0.4053 | 0.6753 | 0.0196 | 0.0397 |
| 0.6201 | 0.6579 | 0.7752 | 0.8165 | 0.0224 | 0.1493 | 0.0090 | 0.3368 | 0.1216 | 0.1344 |
| 0.6724 | 0.6642 | 0.8558 | 0.8302 | 0.0987 | 0.2797 | 0.0481 | 0.5116 | 0.0066 | 0.0181 |
| 0.2376 | 0.4709 | 0.6365 | 0.7782 | 0.0362 | 0.1918 | 0.2685 | 0.6561 | 0.0235 | 0.0444 |
| 0.4955 | 0.6198 | 0.6066 | 0.7706 | 0.1623 | 0.3342 | 0.8459 | 0.8170 | 0.0681 | 0.0891 |
| 0.0818 | 0.3452 | 0.5721 | 0.7622 | 0.4978 | 0.5425 | 0.1144 | 0.5856 | 0.0915 | 0.1092 |
| | | | | | | | | 0.0246 | 0.0461 |
| 0.6949 | 0.6653 | 0.3466 | 0.6614 | 0.5191 | 0.5510 | 0.3466 | 0.6648 | 0.0001 | 0.0012 |
| 0.6363 | 0.6641 | 0.0842 | 0.4917 | 0.5154 | 0.5510 | 0.2692 | 0.6561 | 0.9305 | 0.4787 |
| 0.9234 | 0.7286 | 0.5390 | 0.7501 | 0.9653 | 0.6811 | 0.7068 | 0.7879 | 0.1704 | 0.1720 |
| 0.6550 | 0.6642 | 0.5664 | 0.7622 | 0.5830 | 0.5786 | 0.3252 | 0.6635 | 0.9211 | 0.4756 |
| 0.1213 | 0.3802 | 0.0443 | 0.3767 | 0.0010 | 0.0358 | 0.4844 | 0.7017 | 0.0684 | 0.0891 |
| 0.9771 | 0.7411 | 0.2022 | 0.5977 | 0.8889 | 0.6670 | 0.6008 | 0.7506 | 0.0000 | 0.0000 |
| 0.0818 | 0.3452 | 0.6496 | 0.7782 | 0.1461 | 0.3167 | 0.8622 | 0.8201 | 0.0133 | 0.0305 |
| 0.5000 | 0.6198 | 0.2253 | 0.6090 | 0.0148 | 0.1169 | 0.8874 | 0.8246 | 0.1570 | 0.1640 |
| 0.2198 | 0.4709 | 0.7204 | 0.8004 | 0.1031 | 0.2871 | 0.8821 | 0.8246 | 0.0361 | 0.0604 |
| 0.2273 | 0.4709 | 0.2093 | 0.5977 | 0.2070 | 0.3713 | 0.8564 | 0.8188 | 0.3126 | 0.2476 |
| 0.0860 | 0.3518 | 0.0436 | 0.3767 | 0.5945 | 0.5837 | 0.6807 | 0.7844 | 0.0038 | 0.0139 |
| 0.5097 | 0.6224 | 0.2968 | 0.6533 | 0.8335 | 0.6518 | 0.8819 | 0.8246 | 0.7234 | 0.4181 |
| 0.1572 | 0.4185 | 0.7256 | 0.8004 | 0.4520 | 0.5216 | 0.0837 | 0.5601 | 0.1488 | 0.1568 |
| 0.0083 | 0.2237 | 0.6083 | 0.7706 | 0.7642 | 0.6352 | 0.0622 | 0.5225 | 0.6124 | 0.3754 |
| 0.4919 | 0.6198 | 0.3719 | 0.6705 | 0.7508 | 0.6335 | 0.6879 | 0.7844 | 0.1860 | 0.1822 |
| 0.4612 | 0.6004 | 0.2799 | 0.6478 | 0.0385 | 0.1918 | 0.6341 | 0.7663 | 0.5037 | 0.3359 |
| 0.5156 | 0.6257 | 0.8996 | 0.8441 | 0.2121 | 0.3734 | 0.5907 | 0.7484 | 0.1061 | 0.1233 |
| 0.3876 | 0.5639 | 0.4141 | 0.7007 | 0.4506 | 0.5216 | 0.3188 | 0.6635 | 0.7207 | 0.4176 |
| 0.9596 | 0.7367 | 0.6976 | 0.7941 | 0.0665 | 0.2396 | 0.6991 | 0.7879 | 0.2923 | 0.2373 |
| 0.0120 | 0.2237 | 0.8020 | 0.8177 | 0.1438 | 0.3151 | 0.6093 | 0.7535 | 0.0083 | 0.0216 |
| 0.5456 | 0.6273 | 0.0756 | 0.4788 | 0.7108 | 0.6211 | 0.3336 | 0.6648 | 0.8647 | 0.4604 |
| 0.1760 | 0.4322 | 0.9975 | 0.8533 | 0.5836 | 0.5786 | 0.9832 | 0.8347 | 0.0001 | 0.0009 |
| 0.0056 | 0.2237 | 0.5786 | 0.7622 | 0.0235 | 0.1525 | 0.3251 | 0.6635 | 0.0514 | 0.0756 |
| 0.1166 | 0.3746 | 0.1234 | 0.5031 | 0.2752 | 0.4248 | 0.2024 | 0.6258 | 0.0526 | 0.0763 |
| 0.3461 | 0.5504 | 0.8167 | 0.8179 | 0.9497 | 0.6745 | 0.1512 | 0.6131 | 0.1065 | 0.1233 |
| 0.5528 | 0.6286 | 0.9198 | 0.8444 | 0.9351 | 0.6736 | 0.7604 | 0.8010 | 0.0926 | 0.1101 |
| 0.1572 | 0.4185 | 0.9908 | 0.8533 | 0.0900 | 0.2629 | 0.8642 | 0.8201 | 0.0039 | 0.0141 |
| 0.4589 | 0.6004 | 0.9246 | 0.8444 | 0.9237 | 0.6736 | 0.2455 | 0.6504 | 0.6543 | 0.3942 |
| 0.1657 | 0.4260 | 0.3302 | 0.6550 | 0.2579 | 0.4111 | 0.0766 | 0.5375 | 0.8151 | 0.4478 |
| 0.3816 | 0.5570 | 0.4897 | 0.7234 | 0.3886 | 0.4870 | 0.6676 | 0.7801 | 0.1980 | 0.1884 |
| 0.2306 | 0.4709 | 0.0041 | 0.1828 | 0.0064 | 0.0739 | 0.9043 | 0.8246 | 0.8276 | 0.4515 |
| 0.6167 | 0.6579 | 0.7890 | 0.8177 | 0.5982 | 0.5856 | 0.1275 | 0.5967 | 0.7689 | 0.4346 |
| 0.2458 | 0.4740 | 0.0951 | 0.4999 | 0.0501 | 0.2160 | 0.0498 | 0.5116 | 0.7712 | 0.4347 |
| 0.8771 | 0.7219 | 0.3830 | 0.6811 | 0.2855 | 0.4338 | 0.5040 | 0.7056 | 0.0495 | 0.0742 |
| 0.2667 | 0.4945 | 0.5133 | 0.7350 | 0.0623 | 0.2338 | 0.9278 | 0.8246 | 0.0007 | 0.0046 |
| 0.8614 | 0.7201 | 0.5975 | 0.7700 | 0.9883 | 0.6904 | 0.7222 | 0.7918 | 0.5724 | 0.3593 |
| 0.7268 | 0.6785 | 0.1287 | 0.5071 | 0.2728 | 0.4242 | 0.5143 | 0.7071 | 0.5834 | 0.3630 |
| 0.0090 | 0.2237 | 0.4960 | 0.7261 | 0.1243 | 0.3083 | 0.0727 | 0.5234 | 0.7911 | 0.4415 |
| 0.0100 | 0.2237 | 0.9137 | 0.8444 | 0.3613 | 0.4796 | 0.2746 | 0.6595 | 0.1444 | 0.1533 |
| 0.1097 | 0.3672 | 0.9880 | 0.8533 | 0.1192 | 0.3042 | 0.6460 | 0.7736 | 0.0536 | 0.0771 |
| 0.0019 | 0.1952 | 0.9144 | 0.8444 | 0.0239 | 0.1525 | 0.5577 | 0.7350 | 0.1148 | 0.1298 |
| 0.0205 | 0.2497 | 0.6489 | 0.7782 | 0.3695 | 0.4814 | 0.1579 | 0.6131 | 0.0311 | 0.0531 |
| 0.7794 | 0.6954 | 0.4915 | 0.7234 | 0.9168 | 0.6736 | 0.8477 | 0.8177 | 0.3012 | 0.2411 |
| 0.0055 | 0.2237 | 0.6697 | 0.7821 | 0.0078 | 0.0824 | 0.1688 | 0.6229 | 0.0548 | 0.0774 |
| 0.2015 | 0.4548 | 0.6272 | 0.7782 | 0.1220 | 0.3058 | 0.7816 | 0.8048 | 0.1205 | 0.1344 |
| 0.5711 | 0.6402 | 0.2536 | 0.6326 | 0.3360 | 0.4727 | 0.0464 | 0.5116 | 0.1012 | 0.1185 |
| 0.5092 | 0.6224 | 0.6372 | 0.7782 | 0.6847 | 0.6155 | 0.3039 | 0.6607 | 0.4960 | 0.3322 |
| 0.0521 | 0.3182 | 0.1335 | 0.5071 | 0.4355 | 0.5107 | 0.3868 | 0.6693 | 0.0862 | 0.1047 |
| 0.0008 | 0.1857 | 0.3507 | 0.6627 | 0.0593 | 0.2308 | 0.4274 | 0.6821 | 0.1641 | 0.1683 |
| 0.1611 | 0.4185 | 0.2095 | 0.5977 | 0.8339 | 0.6518 | 0.9090 | 0.8246 | 0.3006 | 0.2411 |
| | | | | | | | | 0.0544 | 0.0771 |
| 0.0595 | 0.3182 | 0.2455 | 0.6307 | 0.3760 | 0.4819 | 0.1454 | 0.6069 | 0.9622 | 0.4866 |
| 0.6891 | 0.6642 | 0.1228 | 0.5031 | 0.0831 | 0.2629 | 0.8397 | 0.8170 | 0.0005 | 0.0040 |
| 0.4613 | 0.6004 | 0.0383 | 0.3763 | 0.1867 | 0.3581 | 0.8139 | 0.8113 | 0.2069 | 0.1931 |
| 0.9811 | 0.7411 | 0.1933 | 0.5977 | 0.6178 | 0.5944 | 0.8426 | 0.8170 | 0.3908 | 0.2880 |
| 0.3495 | 0.5524 | 0.0518 | 0.4026 | 0.1420 | 0.3151 | 0.7987 | 0.8058 | 0.7329 | 0.4231 |
| 0.2063 | 0.4610 | 0.9304 | 0.8444 | 0.6767 | 0.6152 | 0.2173 | 0.6280 | 0.0916 | 0.1092 |
| 0.7422 | 0.6844 | 0.6273 | 0.7782 | 0.9992 | 0.6926 | 0.5131 | 0.7071 | 0.2528 | 0.2188 |
| 0.6251 | 0.6579 | 0.8702 | 0.8353 | 0.1336 | 0.3151 | 0.8048 | 0.8058 | 0.0164 | 0.0353 |
| | | | | | | | | 0.0056 | 0.0168 |
| 0.8022 | 0.7040 | 0.5856 | 0.7622 | 0.1402 | 0.3151 | 0.3365 | 0.6648 | 0.4864 | 0.3293 |
| 0.3170 | 0.5349 | 0.3137 | 0.6550 | 0.5254 | 0.5525 | 0.1455 | 0.6069 | 0.0087 | 0.0224 |
| 0.1228 | 0.3802 | 0.1228 | 0.5031 | 0.8302 | 0.6518 | 0.1228 | 0.5944 | 0.0013 | 0.0072 |
| 0.8442 | 0.7175 | 0.3908 | 0.6899 | 0.3998 | 0.4941 | 0.2480 | 0.6526 | 0.6562 | 0.3948 |
| 0.0764 | 0.3417 | 0.0947 | 0.4999 | 0.3297 | 0.4663 | 0.4308 | 0.6821 | 0.5592 | 0.3561 |
| 0.0428 | 0.3026 | 0.1711 | 0.5676 | 0.7479 | 0.6335 | 0.9862 | 0.8349 | 0.7927 | 0.4419 |
| 0.0793 | 0.3430 | 0.5459 | 0.7501 | 0.6359 | 0.6001 | 0.5564 | 0.7345 | 0.0041 | 0.0141 |
| 0.0896 | 0.3523 | 0.2679 | 0.6441 | 0.5388 | 0.5589 | 0.8906 | 0.8246 | 0.0506 | 0.0749 |
| 0.6480 | 0.6641 | 0.8038 | 0.8177 | 0.0448 | 0.2035 | 0.2103 | 0.6280 | 0.0587 | 0.0818 |
| 0.0348 | 0.2863 | 0.0075 | 0.2084 | 0.0068 | 0.0760 | 0.1257 | 0.5944 | 0.3466 | 0.2653 |
| 0.1431 | 0.4142 | 0.1848 | 0.5832 | 0.3566 | 0.4796 | 0.7403 | 0.7941 | 0.6428 | 0.3883 |
| 0.8763 | 0.7219 | 0.8270 | 0.8229 | 0.1484 | 0.3185 | 0.5562 | 0.7345 | 0.0515 | 0.0756 |
| 0.8278 | 0.7149 | 0.7364 | 0.8037 | 0.6087 | 0.5883 | 0.9733 | 0.8347 | 0.2145 | 0.1985 |
| 0.5471 | 0.6273 | 0.4767 | 0.7213 | 0.2903 | 0.4354 | 0.5993 | 0.7506 | 0.4686 | 0.3248 |
| 0.3325 | 0.5448 | 0.0709 | 0.4642 | 0.4003 | 0.4941 | 0.3346 | 0.6648 | 0.0371 | 0.0612 |
| 0.0178 | 0.2407 | 0.7925 | 0.8177 | 0.3845 | 0.4847 | 0.1801 | 0.6258 | 0.0002 | 0.0020 |
| 0.3463 | 0.5504 | 0.3281 | 0.6550 | 0.9308 | 0.6736 | 0.0908 | 0.5648 | 0.0029 | 0.0117 |
| 0.6513 | 0.6641 | 0.5911 | 0.7629 | 0.0558 | 0.2294 | 0.2946 | 0.6604 | 0.0268 | 0.0490 |
| 0.0696 | 0.3270 | 0.1222 | 0.5031 | 0.0071 | 0.0766 | 0.1172 | 0.5885 | 0.8579 | 0.4604 |
| 0.6615 | 0.6642 | 0.0221 | 0.2928 | 0.0470 | 0.2088 | 0.9420 | 0.8280 | 0.0221 | 0.0435 |
| 0.9862 | 0.7417 | 0.4391 | 0.7007 | 0.0514 | 0.2160 | 0.7371 | 0.7941 | 0.0057 | 0.0168 |
| 0.0526 | 0.3182 | 0.1819 | 0.5796 | 0.0000 | 0.0046 | 0.3305 | 0.6648 | 0.1132 | 0.1288 |
| 0.1101 | 0.3672 | 0.3107 | 0.6550 | 0.0511 | 0.2160 | 0.7913 | 0.8058 | 0.0467 | 0.0713 |
| 0.0109 | 0.2237 | 0.1744 | 0.5741 | 0.8237 | 0.6484 | 0.7466 | 0.7941 | 0.4654 | 0.3239 |
| 0.5924 | 0.6522 | 0.7315 | 0.8011 | 0.0733 | 0.2484 | 0.3983 | 0.6735 | 0.0221 | 0.0435 |
| 0.9282 | 0.7294 | 0.8265 | 0.8229 | 0.1254 | 0.3083 | 0.6533 | 0.7778 | 0.0077 | 0.0202 |
| 0.7230 | 0.6785 | 0.7451 | 0.8088 | 0.0534 | 0.2215 | 0.5502 | 0.7345 | 0.9780 | 0.4911 |
| 0.0632 | 0.3199 | 0.1059 | 0.4999 | 0.0056 | 0.0722 | 0.0137 | 0.3604 | 0.0381 | 0.0612 |
| 0.0968 | 0.3626 | 0.0216 | 0.2928 | 0.0421 | 0.1982 | 0.1612 | 0.6166 | 0.0007 | 0.0046 |
| 0.7683 | 0.6924 | 0.8059 | 0.8177 | 0.9206 | 0.6736 | 0.1861 | 0.6258 | 0.7052 | 0.4130 |
| 0.0711 | 0.3285 | 0.0412 | 0.3763 | 0.0022 | 0.0498 | 0.0924 | 0.5648 | 0.4850 | 0.3292 |
| 0.0668 | 0.3242 | 0.4639 | 0.7101 | 0.5253 | 0.5525 | 0.4553 | 0.6890 | 0.0011 | 0.0059 |
| 0.0755 | 0.3417 | 0.0641 | 0.4455 | 0.0030 | 0.0549 | 0.0945 | 0.5648 | 0.9948 | 0.4951 |
| 0.8055 | 0.7040 | 0.9817 | 0.8530 | 0.6569 | 0.6121 | 0.9974 | 0.8421 | 0.2347 | 0.2112 |
| 0.2197 | 0.4709 | 0.0993 | 0.4999 | 0.0166 | 0.1257 | 0.7111 | 0.7885 | 0.6577 | 0.3952 |
| 0.0232 | 0.2518 | 0.0323 | 0.3518 | 0.2181 | 0.3764 | 0.0543 | 0.5116 | 0.0009 | 0.0055 |
| 0.3637 | 0.5570 | 0.1063 | 0.4999 | 0.1882 | 0.3581 | 0.8422 | 0.8170 | 0.3594 | 0.2714 |
| 0.0213 | 0.2497 | 0.0764 | 0.4788 | 0.7076 | 0.6201 | 0.0201 | 0.3996 | 0.0934 | 0.1108 |
| 0.7024 | 0.6696 | 0.4473 | 0.7043 | 0.8987 | 0.6704 | 0.9775 | 0.8347 | 0.0000 | 0.0000 |
| 0.3797 | 0.5570 | 0.2511 | 0.6323 | 0.7758 | 0.6352 | 0.7297 | 0.7936 | 0.0138 | 0.0312 |
| 0.4563 | 0.6004 | 0.3352 | 0.6550 | 0.7118 | 0.6211 | 0.1850 | 0.6258 | 0.0000 | 0.0006 |
| 0.0159 | 0.2237 | 0.0111 | 0.2328 | 0.2752 | 0.4248 | 0.3249 | 0.6635 | 0.5681 | 0.3584 |
| 0.4662 | 0.6028 | 0.5646 | 0.7617 | 0.9178 | 0.6736 | 0.7648 | 0.8016 | 0.0001 | 0.0013 |
| 0.3679 | 0.5570 | 0.6085 | 0.7706 | 0.1918 | 0.3598 | 0.4763 | 0.7017 | 0.7745 | 0.4358 |
| 0.4500 | 0.5999 | 0.8830 | 0.8408 | 0.0717 | 0.2465 | 0.1447 | 0.6069 | 0.8282 | 0.4515 |
| 0.0806 | 0.3442 | 0.1102 | 0.5011 | 0.0098 | 0.0944 | 0.3601 | 0.6648 | 0.0687 | 0.0891 |
| 0.4834 | 0.6133 | 0.9320 | 0.8444 | 0.9165 | 0.6736 | 0.3202 | 0.6635 | 0.0061 | 0.0176 |
| 0.0150 | 0.2237 | 0.0879 | 0.4917 | 0.2898 | 0.4354 | 0.4773 | 0.7017 | 0.5915 | 0.3665 |
| 0.7087 | 0.6724 | 0.2292 | 0.6110 | 0.1853 | 0.3581 | 0.4933 | 0.7056 | 0.6879 | 0.4064 |
| 0.0676 | 0.3260 | 0.0332 | 0.3519 | 0.0771 | 0.2530 | 0.9791 | 0.8347 | 0.1817 | 0.1800 |
| 0.2865 | 0.5116 | 0.6650 | 0.7800 | 0.1440 | 0.3151 | 0.1832 | 0.6258 | 0.0000 | 0.0000 |
| 0.3436 | 0.5504 | 0.5034 | 0.7316 | 0.3328 | 0.4696 | 0.0599 | 0.5136 | 0.2553 | 0.2192 |
| 0.2477 | 0.4741 | 0.1000 | 0.4999 | 0.8714 | 0.6635 | 0.0542 | 0.5116 | 0.0027 | 0.0111 |
| 0.5532 | 0.6286 | 0.4922 | 0.7234 | 0.6494 | 0.6073 | 0.7603 | 0.8010 | 0.0506 | 0.0749 |
| 0.9124 | 0.7281 | 0.7840 | 0.8177 | 0.5660 | 0.5730 | 0.9308 | 0.8246 | 0.5380 | 0.3462 |
| 0.1081 | 0.3668 | 0.2063 | 0.5977 | 0.7829 | 0.6379 | 0.8026 | 0.8058 | 0.4898 | 0.3298 |
| 0.5291 | 0.6262 | 0.8001 | 0.8177 | 0.8819 | 0.6670 | 0.1262 | 0.5944 | 0.0046 | 0.0148 |
| 0.3646 | 0.5570 | 0.2519 | 0.6323 | 0.1891 | 0.3581 | 0.7908 | 0.8058 | 0.9063 | 0.4712 |
| 0.8934 | 0.7230 | 0.3005 | 0.6544 | 0.4146 | 0.4992 | 0.9750 | 0.8347 | 0.0000 | 0.0001 |
| 0.8408 | 0.7175 | 0.9427 | 0.8465 | 0.7582 | 0.6335 | 0.7609 | 0.8010 | 0.0014 | 0.0072 |
| | | 0.3559 | 0.6631 | | | | | 0.0001 | 0.0009 |
| 0.1239 | 0.3805 | 0.2803 | 0.6478 | 0.9427 | 0.6736 | 0.3456 | 0.6648 | 0.1518 | 0.1593 |
| 0.7142 | 0.6724 | 0.2989 | 0.6533 | 0.1152 | 0.2984 | 0.0471 | 0.5116 | 0.2812 | 0.2328 |
| 0.9429 | 0.7349 | 0.9921 | 0.8533 | 0.5938 | 0.5837 | 0.4362 | 0.6835 | 0.8647 | 0.4604 |
| 0.3466 | 0.5504 | 0.3466 | 0.6614 | 0.3466 | 0.4742 | 0.3466 | 0.6648 | 0.0000 | 0.0000 |
| 0.3466 | 0.5504 | 0.3466 | 0.6614 | 0.5560 | 0.5684 | 0.3466 | 0.6648 | 0.0003 | 0.0022 |
| 0.0480 | 0.3055 | 0.0201 | 0.2928 | 0.0673 | 0.2410 | 0.9351 | 0.8268 | 0.0048 | 0.0152 |
| 0.8115 | 0.7058 | 0.6800 | 0.7857 | 0.4683 | 0.5264 | 0.0701 | 0.5225 | 0.3960 | 0.2912 |
| 0.1662 | 0.4260 | 0.9532 | 0.8469 | 0.6446 | 0.6046 | 0.3799 | 0.6652 | 0.7064 | 0.4130 |
| 0.4315 | 0.5919 | 0.3280 | 0.6550 | 0.2364 | 0.3980 | 0.8510 | 0.8177 | 0.7079 | 0.4130 |
| 0.1021 | 0.3666 | 0.0168 | 0.2839 | 0.0262 | 0.1623 | 0.7131 | 0.7885 | 0.5068 | 0.3370 |
| 0.9212 | 0.7281 | 0.6022 | 0.7706 | 0.2872 | 0.4338 | 0.6237 | 0.7619 | 0.7584 | 0.4315 |
| 0.8495 | 0.7193 | 0.1465 | 0.5270 | 0.8424 | 0.6536 | 0.9486 | 0.8291 | 0.0040 | 0.0141 |
| 0.9309 | 0.7298 | 0.2832 | 0.6496 | 0.4231 | 0.5059 | 0.1546 | 0.6131 | 0.3210 | 0.2512 |
| 0.9305 | 0.7298 | 0.5276 | 0.7446 | 0.1545 | 0.3248 | 0.4063 | 0.6753 | 0.0025 | 0.0107 |
| 0.6614 | 0.6642 | 0.9340 | 0.8446 | 0.2276 | 0.3886 | 0.9260 | 0.8246 | 0.6213 | 0.3782 |
| 0.3523 | 0.5537 | 0.6625 | 0.7800 | 0.1760 | 0.3535 | 0.2196 | 0.6280 | 0.5298 | 0.3439 |
| 0.7810 | 0.6954 | 0.9574 | 0.8469 | 0.4325 | 0.5107 | 0.5807 | 0.7422 | 0.5597 | 0.3561 |
| 0.4215 | 0.5830 | 0.4005 | 0.6946 | 0.9671 | 0.6817 | 0.7125 | 0.7885 | 0.0000 | 0.0000 |
| 0.5685 | 0.6395 | 0.0826 | 0.4917 | 0.0213 | 0.1450 | 0.8429 | 0.8170 | 0.3887 | 0.2874 |
| 0.0975 | 0.3626 | 0.3299 | 0.6550 | 0.3074 | 0.4471 | 0.1937 | 0.6258 | 0.0224 | 0.0436 |
| 0.8852 | 0.7219 | 0.8762 | 0.8353 | 0.8126 | 0.6465 | 0.9457 | 0.8288 | 0.3905 | 0.2880 |
| 0.7865 | 0.6966 | 0.0783 | 0.4804 | 0.2718 | 0.4242 | 0.2717 | 0.6595 | 0.0866 | 0.1047 |
| 0.6784 | 0.6642 | 0.5771 | 0.7622 | 0.2539 | 0.4095 | 0.2806 | 0.6595 | 0.0123 | 0.0291 |
| 0.1059 | 0.3668 | 0.2087 | 0.5977 | 0.2003 | 0.3664 | 0.2975 | 0.6604 | 0.0831 | 0.1036 |
| 0.2320 | 0.4709 | 0.0642 | 0.4455 | 0.7535 | 0.6335 | 0.9135 | 0.8246 | 0.0275 | 0.0498 |
| 0.6824 | 0.6642 | 0.5436 | 0.7501 | 0.0377 | 0.1918 | 0.3641 | 0.6648 | 0.0670 | 0.0887 |
| 0.2224 | 0.4709 | 0.6688 | 0.7821 | 0.0394 | 0.1921 | 0.6624 | 0.7801 | 0.1410 | 0.1507 |
| 0.4354 | 0.5938 | 0.4337 | 0.7007 | 0.5002 | 0.5441 | 0.1439 | 0.6069 | 0.0021 | 0.0096 |
| 0.5358 | 0.6273 | 0.9301 | 0.8444 | 0.8080 | 0.6465 | 0.9007 | 0.8246 | 0.0310 | 0.0531 |
| 0.9197 | 0.7281 | 0.8755 | 0.8353 | 0.0321 | 0.1796 | 0.3107 | 0.6635 | 0.8579 | 0.4604 |
| 0.7448 | 0.6849 | 0.4442 | 0.7041 | 0.4077 | 0.4992 | 0.9803 | 0.8347 | 0.3578 | 0.2707 |
| 0.6258 | 0.6579 | 0.9313 | 0.8444 | 0.2965 | 0.4402 | 0.3041 | 0.6607 | 0.0867 | 0.1047 |
| 0.4034 | 0.5710 | 0.3975 | 0.6946 | 0.6784 | 0.6152 | 0.4869 | 0.7023 | 0.0002 | 0.0015 |
| 0.5419 | 0.6273 | 0.9715 | 0.8497 | 0.9762 | 0.6850 | 0.5491 | 0.7345 | 0.4215 | 0.3023 |
| 0.5855 | 0.6479 | 0.0897 | 0.4917 | 0.3765 | 0.4819 | 0.2189 | 0.6280 | 0.0007 | 0.0046 |
| 0.6785 | 0.6642 | 0.4613 | 0.7101 | 0.0653 | 0.2365 | 0.5401 | 0.7281 | 0.1726 | 0.1728 |
| 0.6243 | 0.6579 | 0.0087 | 0.2084 | 0.1437 | 0.3151 | 0.6910 | 0.7857 | 0.5681 | 0.3584 |
| 0.0697 | 0.3270 | 0.1055 | 0.4999 | 0.1075 | 0.2942 | 0.4142 | 0.6821 | 0.0031 | 0.0122 |
| 0.2070 | 0.4610 | 0.1694 | 0.5645 | 0.5035 | 0.5447 | 0.3606 | 0.6648 | 0.0754 | 0.0969 |
| 0.1223 | 0.3802 | 0.8249 | 0.8229 | 0.3820 | 0.4833 | 0.5976 | 0.7506 | 0.7084 | 0.4130 |
| 0.0461 | 0.3055 | 0.0809 | 0.4884 | 0.0319 | 0.1796 | 0.0214 | 0.3996 | 0.2742 | 0.2279 |
| | | | | | | | | 0.0285 | 0.0506 |
| 0.9853 | 0.7417 | 0.2418 | 0.6241 | 0.7555 | 0.6335 | 0.7229 | 0.7918 | 0.2199 | 0.2023 |
| 0.8605 | 0.7201 | 0.0432 | 0.3767 | 0.0354 | 0.1918 | 0.2652 | 0.6561 | 0.3515 | 0.2677 |
| 0.0870 | 0.3518 | 0.0788 | 0.4804 | 0.0091 | 0.0921 | 0.2218 | 0.6290 | 0.0208 | 0.0418 |
| 0.2233 | 0.4709 | 0.6425 | 0.7782 | 0.1957 | 0.3624 | 0.3391 | 0.6648 | 0.9666 | 0.4880 |
| 0.7205 | 0.6773 | 0.6143 | 0.7747 | 0.0068 | 0.0760 | 0.9202 | 0.8246 | 0.1667 | 0.1703 |
| 0.5250 | 0.6262 | 0.9989 | 0.8533 | 0.0132 | 0.1128 | 0.0124 | 0.3368 | 0.9722 | 0.4898 |
| 0.1133 | 0.3697 | 0.0400 | 0.3763 | 0.0164 | 0.1257 | 0.8028 | 0.8058 | 0.0006 | 0.0040 |
| 0.7243 | 0.6785 | 0.1290 | 0.5071 | 0.0858 | 0.2629 | 0.0124 | 0.3368 | 0.1715 | 0.1725 |
| 0.0633 | 0.3199 | 0.0155 | 0.2839 | 0.0610 | 0.2329 | 0.3245 | 0.6635 | 0.0436 | 0.0679 |
| 0.1240 | 0.3805 | 0.7233 | 0.8004 | 0.4586 | 0.5239 | 0.0283 | 0.4590 | 0.9957 | 0.4951 |
| 0.2251 | 0.4709 | 0.5424 | 0.7501 | 0.9875 | 0.6904 | 0.0778 | 0.5395 | 0.9617 | 0.4866 |
| 0.9931 | 0.7431 | 0.9957 | 0.8533 | 0.2191 | 0.3772 | 0.9632 | 0.8347 | 0.0117 | 0.0281 |
| 0.8812 | 0.7219 | 0.0986 | 0.4999 | 0.9325 | 0.6736 | 0.7005 | 0.7879 | 0.1664 | 0.1703 |
| 0.1854 | 0.4361 | 0.5729 | 0.7622 | 0.3794 | 0.4819 | 0.5068 | 0.7061 | 0.0062 | 0.0176 |
| 0.8427 | 0.7175 | 0.6984 | 0.7941 | 0.1632 | 0.3342 | 0.6839 | 0.7844 | 0.9756 | 0.4907 |
| 0.1098 | 0.3672 | 0.2575 | 0.6326 | 0.0108 | 0.0965 | 0.0401 | 0.5116 | 0.0038 | 0.0138 |
| 0.9149 | 0.7281 | 0.9748 | 0.8504 | 0.6671 | 0.6147 | 0.9710 | 0.8347 | 0.5745 | 0.3594 |
| 0.9947 | 0.7431 | 0.8108 | 0.8177 | 0.0430 | 0.1995 | 0.3626 | 0.6648 | 0.9900 | 0.4951 |
| 0.2486 | 0.4741 | 0.2100 | 0.5977 | 0.3613 | 0.4796 | 0.8266 | 0.8167 | 0.5784 | 0.3613 |
| 0.1690 | 0.4260 | 0.1224 | 0.5031 | 0.1480 | 0.3185 | 0.9698 | 0.8347 | 0.1587 | 0.1646 |
| 0.4587 | 0.6004 | 0.8131 | 0.8177 | 0.3783 | 0.4819 | 0.9016 | 0.8246 | 0.2366 | 0.2123 |
| 0.8453 | 0.7175 | 0.1232 | 0.5031 | 0.8838 | 0.6670 | 0.7388 | 0.7941 | 0.2831 | 0.2328 |
| 0.5297 | 0.6262 | 0.2198 | 0.6071 | 0.2396 | 0.3982 | 0.3169 | 0.6635 | 0.8146 | 0.4478 |
| 0.8748 | 0.7219 | 0.6640 | 0.7800 | 0.5197 | 0.5510 | 0.3656 | 0.6648 | 0.2843 | 0.2328 |
| 0.0428 | 0.3026 | 0.9327 | 0.8444 | 0.7428 | 0.6307 | 0.3998 | 0.6735 | 0.3689 | 0.2768 |
| 0.0579 | 0.3182 | 0.2585 | 0.6326 | 0.2172 | 0.3760 | 0.6667 | 0.7801 | 0.8627 | 0.4604 |
| 0.3767 | 0.5570 | 0.1152 | 0.5031 | 0.2452 | 0.4043 | 0.0695 | 0.5225 | 0.0322 | 0.0545 |
| 0.0469 | 0.3055 | 0.9454 | 0.8469 | 0.7032 | 0.6196 | 0.0490 | 0.5116 | 0.0311 | 0.0531 |
| 0.0125 | 0.2237 | 0.6075 | 0.7706 | 0.2379 | 0.3980 | 0.1534 | 0.6131 | 0.0023 | 0.0102 |
| 0.6196 | 0.6579 | 0.3575 | 0.6644 | 0.9564 | 0.6771 | 0.7826 | 0.8048 | 0.6930 | 0.4088 |
| 0.0231 | 0.2518 | 0.3556 | 0.6631 | 0.0006 | 0.0255 | 0.2381 | 0.6397 | 0.0470 | 0.0715 |
| 0.6230 | 0.6579 | 0.4471 | 0.7043 | 0.1629 | 0.3342 | 0.1950 | 0.6258 | 0.4237 | 0.3029 |
| 0.0320 | 0.2669 | 0.0400 | 0.3763 | 0.0279 | 0.1678 | 0.6702 | 0.7805 | 0.2465 | 0.2167 |
| 0.7478 | 0.6849 | 0.1362 | 0.5085 | 0.3750 | 0.4819 | 0.1035 | 0.5714 | 0.0127 | 0.0295 |
| 0.8989 | 0.7249 | 0.4334 | 0.7007 | 0.6233 | 0.5957 | 0.0395 | 0.5116 | 0.1212 | 0.1344 |
| 0.5181 | 0.6257 | 0.6630 | 0.7800 | 0.6841 | 0.6155 | 0.9115 | 0.8246 | 0.9337 | 0.4787 |
| 0.3812 | 0.5570 | 0.5101 | 0.7340 | 0.5481 | 0.5654 | 0.1389 | 0.6069 | 0.0536 | 0.0771 |
| 0.6690 | 0.6642 | 0.2787 | 0.6478 | 0.3082 | 0.4471 | 0.0462 | 0.5116 | 0.0972 | 0.1150 |
| 0.7137 | 0.6724 | 0.9106 | 0.8444 | 0.2354 | 0.3976 | 0.3854 | 0.6693 | 0.3191 | 0.2510 |
| 0.0625 | 0.3199 | 0.1348 | 0.5071 | 0.2112 | 0.3734 | 0.1015 | 0.5714 | 0.0984 | 0.1161 |
| 0.4805 | 0.6122 | 0.6751 | 0.7856 | 0.0184 | 0.1354 | 0.7703 | 0.8025 | 0.1330 | 0.1449 |
| 0.4660 | 0.6028 | 0.6367 | 0.7782 | 0.2342 | 0.3967 | 0.1338 | 0.6069 | 0.1567 | 0.1640 |
| 0.8946 | 0.7230 | 0.2686 | 0.6441 | 0.1111 | 0.2946 | 0.2977 | 0.6604 | 0.6225 | 0.3782 |
| 0.1715 | 0.4288 | 0.9380 | 0.8447 | 0.7488 | 0.6335 | 0.2683 | 0.6561 | 0.8921 | 0.4665 |
| 0.6748 | 0.6642 | 0.2763 | 0.6478 | 0.6935 | 0.6181 | 0.0463 | 0.5116 | 0.1908 | 0.1841 |
| 0.1843 | 0.4361 | 0.4912 | 0.7234 | 0.7909 | 0.6398 | 0.0598 | 0.5136 | 0.3982 | 0.2916 |
| 0.7328 | 0.6803 | 0.6320 | 0.7782 | 0.4460 | 0.5191 | 0.2280 | 0.6375 | 0.1189 | 0.1337 |
| 0.6663 | 0.6642 | 0.8062 | 0.8177 | 0.6914 | 0.6181 | 0.5622 | 0.7370 | 0.2732 | 0.2275 |
| 0.1028 | 0.3668 | 0.0090 | 0.2084 | 0.0014 | 0.0375 | 0.2165 | 0.6280 | 0.7465 | 0.4272 |
| 0.6447 | 0.6641 | 0.1624 | 0.5502 | 0.1383 | 0.3151 | 0.6654 | 0.7801 | 0.5128 | 0.3375 |
| 0.0663 | 0.3241 | 0.1625 | 0.5502 | 0.0576 | 0.2296 | 0.5083 | 0.7061 | 0.5326 | 0.3444 |
| 0.1057 | 0.3668 | 0.9263 | 0.8444 | 0.6982 | 0.6188 | 0.6622 | 0.7801 | 0.4091 | 0.2963 |
| 0.1498 | 0.4179 | 0.1557 | 0.5448 | 0.5766 | 0.5780 | 0.2541 | 0.6561 | 0.0795 | 0.1006 |
| 0.8048 | 0.7040 | 0.9878 | 0.8533 | 0.7280 | 0.6254 | 0.8880 | 0.8246 | 0.8660 | 0.4604 |
| 0.4935 | 0.6198 | 0.6529 | 0.7782 | 0.8227 | 0.6484 | 0.6862 | 0.7844 | 0.8451 | 0.4577 |
| 0.4193 | 0.5824 | 0.5086 | 0.7340 | 0.8528 | 0.6554 | 0.4818 | 0.7017 | 0.9676 | 0.4880 |
| 0.4153 | 0.5792 | 0.2498 | 0.6323 | 0.9457 | 0.6745 | 0.2785 | 0.6595 | 0.8768 | 0.4643 |
| 0.5076 | 0.6224 | 0.2137 | 0.6009 | 0.9335 | 0.6736 | 0.2285 | 0.6375 | 0.7751 | 0.4358 |
| 0.8799 | 0.7219 | 0.9948 | 0.8533 | 0.9476 | 0.6745 | 0.7007 | 0.7879 | 0.8642 | 0.4604 |
| 0.5673 | 0.6393 | 0.8332 | 0.8269 | 0.6376 | 0.6008 | 0.7893 | 0.8058 | 0.0569 | 0.0798 |
| 0.3685 | 0.5570 | 0.5840 | 0.7622 | 0.8418 | 0.6536 | 0.9440 | 0.8288 | 0.0126 | 0.0295 |
| 0.4656 | 0.6028 | 0.8752 | 0.8353 | 0.3441 | 0.4742 | 0.5554 | 0.7345 | 0.2536 | 0.2188 |
| 0.3785 | 0.5570 | 0.3788 | 0.6779 | 0.0212 | 0.1450 | 0.0530 | 0.5116 | 0.1080 | 0.1236 |
| 0.9203 | 0.7281 | 0.3515 | 0.6627 | 0.4147 | 0.4992 | 0.4053 | 0.6753 | 0.8797 | 0.4643 |
| 0.9867 | 0.7417 | 0.0586 | 0.4396 | 0.0889 | 0.2629 | 0.6183 | 0.7573 | 0.1072 | 0.1233 |
| 0.5449 | 0.6273 | 0.3473 | 0.6614 | 0.6584 | 0.6121 | 0.1866 | 0.6258 | 0.0000 | 0.0007 |
| 0.0432 | 0.3026 | 0.0250 | 0.2973 | 0.0006 | 0.0255 | 0.2171 | 0.6280 | 0.0000 | 0.0001 |
| 0.0646 | 0.3199 | 0.0521 | 0.4026 | 0.2206 | 0.3787 | 0.2161 | 0.6280 | 0.6742 | 0.4030 |
| 0.1344 | 0.4035 | 0.3669 | 0.6705 | 0.6288 | 0.5970 | 0.1634 | 0.6171 | 0.3370 | 0.2593 |
| 0.6222 | 0.6579 | 0.1092 | 0.4999 | 0.2411 | 0.3993 | 0.6341 | 0.7663 | 0.8367 | 0.4542 |
| 0.3402 | 0.5493 | 0.7356 | 0.8037 | 0.0010 | 0.0358 | 0.2206 | 0.6280 | 0.1189 | 0.1337 |
| 0.5344 | 0.6273 | 0.8421 | 0.8286 | 0.0104 | 0.0958 | 0.3157 | 0.6635 | 0.0130 | 0.0298 |
| 0.6626 | 0.6642 | 0.7612 | 0.8135 | 0.0011 | 0.0358 | 0.1419 | 0.6069 | 0.0169 | 0.0360 |
| 0.6398 | 0.6641 | 0.7629 | 0.8135 | 0.7337 | 0.6280 | 0.7957 | 0.8058 | 0.0001 | 0.0013 |
| 0.0417 | 0.3026 | 0.0070 | 0.2084 | 0.0214 | 0.1450 | 0.8669 | 0.8201 | 0.2729 | 0.2275 |
| 0.6051 | 0.6577 | 0.4337 | 0.7007 | 0.6921 | 0.6181 | 0.0935 | 0.5648 | 0.3537 | 0.2685 |
| 0.6511 | 0.6641 | 0.7844 | 0.8177 | 0.3881 | 0.4870 | 0.2974 | 0.6604 | 0.2309 | 0.2103 |
| 0.6892 | 0.6642 | 0.9302 | 0.8444 | 0.3734 | 0.4819 | 0.0910 | 0.5648 | 0.8298 | 0.4515 |
| | | | | | | | | 0.0157 | 0.0342 |
| 0.6367 | 0.6641 | 0.6495 | 0.7782 | 0.9927 | 0.6909 | 0.6354 | 0.7663 | 0.0000 | 0.0000 |
| 0.2758 | 0.5031 | 0.8918 | 0.8427 | 0.6811 | 0.6153 | 0.0390 | 0.5116 | 0.8148 | 0.4478 |
| 0.9551 | 0.7367 | 0.0238 | 0.2968 | 0.2006 | 0.3664 | 0.4307 | 0.6821 | 0.1008 | 0.1183 |
| 0.5986 | 0.6550 | 0.6453 | 0.7782 | 0.4846 | 0.5327 | 0.1384 | 0.6069 | 0.4087 | 0.2963 |
| 0.2560 | 0.4813 | 0.0223 | 0.2928 | 0.0193 | 0.1407 | 0.3205 | 0.6635 | 0.0043 | 0.0141 |
| 0.0354 | 0.2879 | 0.0032 | 0.1565 | 0.0004 | 0.0255 | 0.0048 | 0.2636 | 0.3282 | 0.2545 |
| 0.0254 | 0.2518 | 0.0014 | 0.1535 | 0.0029 | 0.0549 | 0.2887 | 0.6600 | 0.2965 | 0.2403 |
| 0.0301 | 0.2616 | 0.1240 | 0.5031 | 0.1722 | 0.3493 | 0.5671 | 0.7388 | 0.8462 | 0.4577 |
| 0.6165 | 0.6579 | 0.5168 | 0.7372 | 0.2992 | 0.4421 | 0.5692 | 0.7388 | 0.0000 | 0.0005 |
| 0.1943 | 0.4446 | 0.1342 | 0.5071 | 0.1317 | 0.3137 | 0.2807 | 0.6595 | 0.2639 | 0.2219 |
| 0.8025 | 0.7040 | 0.1277 | 0.5071 | 0.3687 | 0.4813 | 0.4840 | 0.7017 | 0.1971 | 0.1881 |
| 0.1544 | 0.4185 | 0.4848 | 0.7227 | 0.0859 | 0.2629 | 0.6241 | 0.7619 | 0.0001 | 0.0010 |
| 0.5472 | 0.6273 | 0.5168 | 0.7372 | 0.1537 | 0.3243 | 0.1480 | 0.6116 | 0.0000 | 0.0000 |
| 0.2881 | 0.5130 | 0.5256 | 0.7446 | 0.0060 | 0.0737 | 0.3599 | 0.6648 | 0.7170 | 0.4165 |
| 0.0484 | 0.3055 | 0.9987 | 0.8533 | 0.0005 | 0.0255 | 0.9279 | 0.8246 | 0.3964 | 0.2912 |
| 0.7622 | 0.6915 | 0.9537 | 0.8469 | 0.0839 | 0.2629 | 0.4316 | 0.6821 | 0.0565 | 0.0795 |
| 0.0374 | 0.2966 | 0.1185 | 0.5031 | 0.0008 | 0.0324 | 0.1304 | 0.6031 | 0.2328 | 0.2112 |
| 0.9175 | 0.7281 | 0.0903 | 0.4917 | 0.1393 | 0.3151 | 0.0259 | 0.4387 | 0.6118 | 0.3754 |
| 0.1609 | 0.4185 | 0.3659 | 0.6705 | 0.0450 | 0.2035 | 0.5915 | 0.7484 | 0.1313 | 0.1437 |
| 0.6644 | 0.6642 | 0.0386 | 0.3763 | 0.3582 | 0.4796 | 0.0768 | 0.5375 | 0.0186 | 0.0383 |
| 0.8127 | 0.7060 | 0.0367 | 0.3731 | 0.2537 | 0.4095 | 0.3011 | 0.6607 | 0.8664 | 0.4604 |
| 0.8703 | 0.7219 | 0.2008 | 0.5977 | 0.2018 | 0.3675 | 0.1105 | 0.5853 | 0.0819 | 0.1026 |
| 0.7090 | 0.6724 | 0.4570 | 0.7101 | 0.9539 | 0.6762 | 0.1253 | 0.5944 | 0.7809 | 0.4374 |
| 0.8461 | 0.7175 | 0.3245 | 0.6550 | 0.2867 | 0.4338 | 0.7381 | 0.7941 | 0.0185 | 0.0382 |
| 0.8938 | 0.7230 | 0.2227 | 0.6085 | 0.4134 | 0.4992 | 0.0514 | 0.5116 | 0.0444 | 0.0688 |
| 0.9344 | 0.7310 | 0.9385 | 0.8447 | 0.9328 | 0.6736 | 0.8313 | 0.8170 | 0.2487 | 0.2179 |
| 0.2805 | 0.5062 | 0.4362 | 0.7007 | 0.3411 | 0.4742 | 0.2525 | 0.6561 | 0.4903 | 0.3298 |
| 0.5227 | 0.6260 | 0.8583 | 0.8316 | 0.3907 | 0.4880 | 0.3643 | 0.6648 | 0.0026 | 0.0110 |
| 0.0894 | 0.3523 | 0.4332 | 0.7007 | 0.9318 | 0.6736 | 0.3196 | 0.6635 | 0.0001 | 0.0014 |
| 0.1069 | 0.3668 | 0.4105 | 0.6997 | 0.5594 | 0.5709 | 0.8584 | 0.8197 | 0.0001 | 0.0010 |
| 0.6262 | 0.6579 | 0.6919 | 0.7890 | 0.8621 | 0.6588 | 0.3710 | 0.6648 | 0.6358 | 0.3851 |
| 0.0649 | 0.3199 | 0.7154 | 0.8004 | 0.2936 | 0.4391 | 0.0726 | 0.5234 | 0.3698 | 0.2770 |
| 0.6175 | 0.6579 | 0.7685 | 0.8147 | 0.2151 | 0.3760 | 0.7271 | 0.7936 | 0.4897 | 0.3298 |
| 0.1536 | 0.4185 | 0.7529 | 0.8130 | 0.3076 | 0.4471 | 0.7388 | 0.7941 | 0.7036 | 0.4130 |
| 0.9592 | 0.7367 | 0.5886 | 0.7627 | 0.5481 | 0.5654 | 0.6851 | 0.7844 | 0.0256 | 0.0473 |
| 0.0573 | 0.3182 | 0.2136 | 0.6009 | 0.6701 | 0.6152 | 0.8802 | 0.8246 | 0.6834 | 0.4053 |
| 0.1469 | 0.4179 | 0.9480 | 0.8469 | 0.9301 | 0.6736 | 0.4605 | 0.6929 | 0.4285 | 0.3054 |
| 0.8437 | 0.7175 | 0.7528 | 0.8130 | 0.0378 | 0.1918 | 0.7312 | 0.7936 | 0.8513 | 0.4594 |
| 0.3592 | 0.5547 | 0.7628 | 0.8135 | 0.1378 | 0.3151 | 0.6077 | 0.7535 | 0.1808 | 0.1800 |
| 0.0447 | 0.3046 | 0.0132 | 0.2491 | 0.0513 | 0.2160 | 0.4190 | 0.6821 | 0.0021 | 0.0096 |
| | | | | | | | | 0.0146 | 0.0325 |
| 0.7766 | 0.6952 | 0.6486 | 0.7782 | 0.7545 | 0.6335 | 0.7016 | 0.7879 | 0.2398 | 0.2129 |
| 0.1345 | 0.4035 | 0.2320 | 0.6142 | 0.4729 | 0.5264 | 0.2821 | 0.6595 | 0.0033 | 0.0127 |
| 0.1001 | 0.3640 | 0.4890 | 0.7234 | 0.4719 | 0.5264 | 0.9819 | 0.8347 | 0.0021 | 0.0096 |
| 0.3016 | 0.5249 | 0.6332 | 0.7782 | 0.0238 | 0.1525 | 0.4428 | 0.6869 | 0.1946 | 0.1869 |
| 0.0484 | 0.3055 | 0.6579 | 0.7800 | 0.1122 | 0.2946 | 0.1938 | 0.6258 | 0.4796 | 0.3275 |
| 0.6836 | 0.6642 | 0.1604 | 0.5502 | 0.6195 | 0.5944 | 0.0527 | 0.5116 | 0.1063 | 0.1233 |
| 0.6221 | 0.6579 | 0.1503 | 0.5306 | 0.9282 | 0.6736 | 0.5331 | 0.7215 | 0.0053 | 0.0162 |
| 0.7261 | 0.6785 | 0.3466 | 0.6614 | 0.3466 | 0.4742 | 0.9673 | 0.8347 | 0.7457 | 0.4272 |
| 0.9633 | 0.7367 | 0.6411 | 0.7782 | 0.5264 | 0.5525 | 0.9162 | 0.8246 | 0.2866 | 0.2335 |
| 0.8062 | 0.7040 | 0.7894 | 0.8177 | 0.1401 | 0.3151 | 0.3486 | 0.6648 | 0.3163 | 0.2497 |
| 0.2803 | 0.5062 | 0.3323 | 0.6550 | 0.0648 | 0.2361 | 0.1121 | 0.5856 | 0.5061 | 0.3370 |
| 0.1787 | 0.4322 | 0.0856 | 0.4917 | 0.0877 | 0.2629 | 0.0025 | 0.2605 | 0.0000 | 0.0000 |
| 0.6499 | 0.6641 | 0.7623 | 0.8135 | 0.6955 | 0.6188 | 0.2909 | 0.6600 | 0.0008 | 0.0053 |
| 0.4986 | 0.6198 | 0.5464 | 0.7501 | 0.4609 | 0.5240 | 0.2168 | 0.6280 | 0.0672 | 0.0887 |
| 0.1003 | 0.3640 | 0.4362 | 0.7007 | 0.9428 | 0.6736 | 0.2673 | 0.6561 | 0.1892 | 0.1833 |
| 0.3211 | 0.5392 | 0.3841 | 0.6811 | 0.8843 | 0.6670 | 0.0945 | 0.5648 | 0.0992 | 0.1167 |
| 0.0588 | 0.3182 | 0.4199 | 0.7007 | 0.1157 | 0.2984 | 0.0804 | 0.5451 | 0.1871 | 0.1828 |
| 0.1867 | 0.4375 | 0.8704 | 0.8353 | 0.5127 | 0.5492 | 0.8339 | 0.8170 | 0.0230 | 0.0442 |
| 0.6447 | 0.6641 | 0.3117 | 0.6550 | 0.1233 | 0.3078 | 0.1944 | 0.6258 | 0.9544 | 0.4841 |
| 0.1215 | 0.3802 | 0.7298 | 0.8004 | 0.7775 | 0.6352 | 0.3967 | 0.6735 | 0.1256 | 0.1382 |
| 0.1903 | 0.4413 | 0.3332 | 0.6550 | 0.3585 | 0.4796 | 0.7956 | 0.8058 | 0.0001 | 0.0013 |
| 0.4403 | 0.5967 | 0.4860 | 0.7227 | 0.0397 | 0.1921 | 0.6636 | 0.7801 | 0.7990 | 0.4432 |
| 0.2244 | 0.4709 | 0.7713 | 0.8147 | 0.7767 | 0.6352 | 0.7647 | 0.8016 | 0.0379 | 0.0612 |
| 0.7629 | 0.6915 | 0.1987 | 0.5977 | 0.4159 | 0.4992 | 0.6008 | 0.7506 | 0.0177 | 0.0375 |
| 0.8752 | 0.7219 | 0.9319 | 0.8444 | 0.0367 | 0.1918 | 0.1875 | 0.6258 | 0.0523 | 0.0761 |
| 0.3089 | 0.5330 | 0.6206 | 0.7782 | 0.0138 | 0.1128 | 0.7611 | 0.8010 | 0.2373 | 0.2123 |
| 0.2381 | 0.4709 | 0.1060 | 0.4999 | 0.0003 | 0.0255 | 0.0011 | 0.2465 | 0.0252 | 0.0468 |
| 0.7127 | 0.6724 | 0.8937 | 0.8427 | 0.4094 | 0.4992 | 0.3859 | 0.6693 | 0.3107 | 0.2465 |
| 0.4877 | 0.6173 | 0.1021 | 0.4999 | 0.5108 | 0.5480 | 0.0441 | 0.5116 | 0.9307 | 0.4787 |
| 0.1144 | 0.3701 | 0.0789 | 0.4804 | 0.7394 | 0.6304 | 0.2306 | 0.6375 | 0.3101 | 0.2465 |
| 0.0317 | 0.2669 | 0.2251 | 0.6090 | 0.6486 | 0.6073 | 0.3152 | 0.6635 | 0.0043 | 0.0142 |
| 0.9039 | 0.7281 | 0.8251 | 0.8229 | 0.0762 | 0.2530 | 0.9404 | 0.8276 | 0.4849 | 0.3292 |
| 0.2041 | 0.4576 | 0.9503 | 0.8469 | 0.5042 | 0.5447 | 0.0229 | 0.4066 | 0.0541 | 0.0771 |
| 0.0928 | 0.3558 | 0.6915 | 0.7890 | 0.0600 | 0.2308 | 0.0790 | 0.5427 | 0.2344 | 0.2112 |
| | | | | | | | | 0.0197 | 0.0397 |
| | | | | | | | | 0.0440 | 0.0683 |
| | | | | | | | | 0.0183 | 0.0381 |
| 0.6865 | 0.6642 | 0.5454 | 0.7501 | 0.5539 | 0.5673 | 0.1216 | 0.5944 | 0.0767 | 0.0976 |
| 0.2453 | 0.4740 | 0.9173 | 0.8444 | 0.4616 | 0.5240 | 0.0087 | 0.3368 | 0.1833 | 0.1807 |
| 0.4252 | 0.5863 | 0.1328 | 0.5071 | 0.7754 | 0.6352 | 0.0637 | 0.5225 | 0.2508 | 0.2180 |
| 0.9478 | 0.7353 | 0.3990 | 0.6946 | 0.5315 | 0.5551 | 0.8971 | 0.8246 | 0.9469 | 0.4817 |
| 0.2114 | 0.4679 | 0.0121 | 0.2468 | 0.5691 | 0.5733 | 0.0901 | 0.5648 | 0.5309 | 0.3439 |
| 0.3416 | 0.5504 | 0.0360 | 0.3731 | 0.1884 | 0.3581 | 0.0541 | 0.5116 | 0.5095 | 0.3375 |
| 0.5218 | 0.6260 | 0.4627 | 0.7101 | 0.4680 | 0.5264 | 0.2617 | 0.6561 | 0.0000 | 0.0006 |
| 0.7813 | 0.6954 | 0.4054 | 0.6950 | 0.9688 | 0.6820 | 0.6669 | 0.7801 | 0.9761 | 0.4907 |
| 0.7731 | 0.6952 | 0.6269 | 0.7782 | 0.4553 | 0.5225 | 0.7578 | 0.8010 | 0.9949 | 0.4951 |
| 0.0389 | 0.2985 | 0.1119 | 0.5031 | 0.5287 | 0.5530 | 0.1591 | 0.6131 | 0.2111 | 0.1962 |
| 0.5420 | 0.6273 | 0.1026 | 0.4999 | 0.0258 | 0.1623 | 0.1029 | 0.5714 | 0.7437 | 0.4272 |
| 0.0058 | 0.2237 | 0.2744 | 0.6478 | 0.0025 | 0.0531 | 0.4528 | 0.6869 | 0.0005 | 0.0036 |
| 0.8580 | 0.7196 | 0.8325 | 0.8269 | 0.8035 | 0.6446 | 0.3755 | 0.6648 | 0.7552 | 0.4315 |
| 0.0865 | 0.3518 | 0.0172 | 0.2839 | 0.0039 | 0.0591 | 0.0701 | 0.5225 | 0.9354 | 0.4787 |
| 0.5196 | 0.6257 | 0.4364 | 0.7007 | 0.8433 | 0.6536 | 0.9856 | 0.8349 | 0.0278 | 0.0500 |
| 0.1358 | 0.4035 | 0.2947 | 0.6533 | 0.7170 | 0.6238 | 0.8531 | 0.8177 | 0.4508 | 0.3188 |
| 0.5987 | 0.6550 | 0.6553 | 0.7782 | 0.4796 | 0.5291 | 0.8337 | 0.8170 | 0.0489 | 0.0740 |
| 0.1559 | 0.4185 | 0.7865 | 0.8177 | 0.6317 | 0.5988 | 0.1935 | 0.6258 | 0.8293 | 0.4515 |
| 0.9940 | 0.7431 | 0.6530 | 0.7782 | 0.3780 | 0.4819 | 0.9796 | 0.8347 | 0.0209 | 0.0418 |
| 0.6511 | 0.6641 | 0.4614 | 0.7101 | 0.4768 | 0.5269 | 0.2967 | 0.6604 | 0.0012 | 0.0063 |
| 0.3533 | 0.5537 | 0.4349 | 0.7007 | 0.1875 | 0.3581 | 0.7731 | 0.8037 | 0.0243 | 0.0458 |
| 0.4782 | 0.6122 | 0.6046 | 0.7706 | 0.9590 | 0.6782 | 0.1657 | 0.6172 | 0.1471 | 0.1559 |
| 0.1369 | 0.4035 | 0.9290 | 0.8444 | 0.4846 | 0.5327 | 0.9709 | 0.8347 | 0.1813 | 0.1800 |
| 0.0774 | 0.3417 | 0.4293 | 0.7007 | 0.5024 | 0.5447 | 0.5336 | 0.7215 | 0.0535 | 0.0771 |
| 0.2487 | 0.4741 | 0.4054 | 0.6950 | 0.5743 | 0.5767 | 0.2087 | 0.6280 | 0.0028 | 0.0114 |
| 0.1691 | 0.4260 | 0.5059 | 0.7338 | 0.9941 | 0.6909 | 0.7473 | 0.7941 | 0.0101 | 0.0254 |
| 0.9726 | 0.7393 | 0.7706 | 0.8147 | 0.6629 | 0.6129 | 0.3549 | 0.6648 | 0.0054 | 0.0162 |
| 0.6151 | 0.6579 | 0.1093 | 0.4999 | 0.4537 | 0.5216 | 0.6644 | 0.7801 | 0.0226 | 0.0438 |
| 0.3142 | 0.5343 | 0.6242 | 0.7782 | 0.5953 | 0.5837 | 0.3973 | 0.6735 | 0.0181 | 0.0381 |
| 0.2909 | 0.5154 | 0.8105 | 0.8177 | 0.6721 | 0.6152 | 0.4507 | 0.6869 | 0.0763 | 0.0976 |
| 0.1794 | 0.4325 | 0.6809 | 0.7857 | 0.5906 | 0.5837 | 0.9310 | 0.8246 | 0.1818 | 0.1800 |
| 0.3587 | 0.5547 | 0.0678 | 0.4509 | 0.1102 | 0.2946 | 0.5034 | 0.7056 | 0.2373 | 0.2123 |
| 0.0775 | 0.3417 | 0.4383 | 0.7007 | 0.7914 | 0.6398 | 0.2479 | 0.6526 | 0.6018 | 0.3704 |
| 0.6714 | 0.6642 | 0.4303 | 0.7007 | 0.2382 | 0.3980 | 0.5952 | 0.7506 | 0.0623 | 0.0847 |
| 0.0942 | 0.3584 | 0.7282 | 0.8004 | 0.6754 | 0.6152 | 0.4215 | 0.6821 | 0.2793 | 0.2317 |
| 0.7762 | 0.6952 | 0.6803 | 0.7857 | 0.7101 | 0.6211 | 0.6828 | 0.7844 | 0.0031 | 0.0124 |
| 0.0458 | 0.3055 | 0.5233 | 0.7446 | 0.2962 | 0.4402 | 0.6811 | 0.7844 | 0.1635 | 0.1680 |
| 0.5288 | 0.6262 | 0.2103 | 0.5977 | 0.0455 | 0.2035 | 0.2637 | 0.6561 | 0.7691 | 0.4346 |
| 0.3967 | 0.5681 | 0.6525 | 0.7782 | 0.1254 | 0.3083 | 0.2029 | 0.6258 | 0.2641 | 0.2219 |
| 0.8556 | 0.7193 | 0.8904 | 0.8427 | 0.9398 | 0.6736 | 0.2527 | 0.6561 | 0.0159 | 0.0344 |
| 0.4827 | 0.6133 | 0.7382 | 0.8038 | 0.2484 | 0.4053 | 0.8020 | 0.8058 | 0.0000 | 0.0007 |
| 0.9347 | 0.7310 | 0.9698 | 0.8492 | 0.8560 | 0.6565 | 0.2571 | 0.6561 | 0.3162 | 0.2497 |
| 0.3008 | 0.5249 | 0.3672 | 0.6705 | 0.1966 | 0.3624 | 0.3597 | 0.6648 | 0.4121 | 0.2975 |
| 0.4487 | 0.5997 | 0.2199 | 0.6071 | 0.1385 | 0.3151 | 0.4418 | 0.6869 | 0.8607 | 0.4604 |
| 0.1590 | 0.4185 | 0.0768 | 0.4788 | 0.1859 | 0.3581 | 0.0992 | 0.5714 | 0.4639 | 0.3236 |
| 0.2186 | 0.4709 | 0.9641 | 0.8486 | 0.6187 | 0.5944 | 0.2908 | 0.6600 | 0.0006 | 0.0040 |
| 0.7678 | 0.6924 | 0.6739 | 0.7856 | 0.7346 | 0.6280 | 0.5300 | 0.7209 | 0.3011 | 0.2411 |
| 0.9446 | 0.7353 | 0.7968 | 0.8177 | 0.7889 | 0.6398 | 0.9538 | 0.8308 | 0.1726 | 0.1728 |
| 0.7571 | 0.6906 | 0.4025 | 0.6946 | 0.4897 | 0.5355 | 0.4835 | 0.7017 | 0.3370 | 0.2593 |
| 0.2382 | 0.4709 | 0.0082 | 0.2084 | 0.4092 | 0.4992 | 0.7837 | 0.8048 | 0.2532 | 0.2188 |
| 0.4017 | 0.5708 | 0.3112 | 0.6550 | 0.0500 | 0.2160 | 0.2796 | 0.6595 | 0.0010 | 0.0057 |
| 0.0868 | 0.3518 | 0.0058 | 0.2084 | 0.4727 | 0.5264 | 0.5611 | 0.7370 | 0.0629 | 0.0847 |
| 0.1448 | 0.4142 | 0.1303 | 0.5071 | 0.0579 | 0.2296 | 0.0030 | 0.2605 | 0.0522 | 0.0761 |
| 0.1882 | 0.4380 | 0.1834 | 0.5810 | 0.0811 | 0.2607 | 0.4714 | 0.7014 | 0.0088 | 0.0226 |
| 0.1759 | 0.4322 | 0.4109 | 0.6997 | 0.3247 | 0.4614 | 0.3443 | 0.6648 | 0.4231 | 0.3029 |
| 0.3121 | 0.5330 | 0.5424 | 0.7501 | 0.1095 | 0.2946 | 0.3508 | 0.6648 | 0.5160 | 0.3386 |
| 0.3236 | 0.5407 | 0.9574 | 0.8469 | 0.3464 | 0.4742 | 0.3453 | 0.6648 | 0.4901 | 0.3298 |
| 0.8849 | 0.7219 | 0.1351 | 0.5071 | 0.8919 | 0.6685 | 0.2733 | 0.6595 | 0.1146 | 0.1298 |
| 0.6540 | 0.6642 | 0.7791 | 0.8174 | 0.5079 | 0.5459 | 0.8973 | 0.8246 | 0.4173 | 0.2998 |
| 0.2973 | 0.5226 | 0.1813 | 0.5796 | 0.0031 | 0.0549 | 0.5673 | 0.7388 | 0.2034 | 0.1914 |
| 0.0857 | 0.3518 | 0.0169 | 0.2839 | 0.0022 | 0.0498 | 0.3129 | 0.6635 | 0.3750 | 0.2800 |
| 0.6655 | 0.6642 | 0.2065 | 0.5977 | 0.7776 | 0.6352 | 0.4006 | 0.6735 | 0.1832 | 0.1807 |
| 0.1830 | 0.4361 | 0.8436 | 0.8286 | 0.8463 | 0.6538 | 0.3499 | 0.6648 | 0.4866 | 0.3293 |
| 0.5399 | 0.6273 | 0.1144 | 0.5031 | 0.4355 | 0.5107 | 0.4750 | 0.7017 | 0.3707 | 0.2772 |
| 0.3311 | 0.5439 | 0.7216 | 0.8004 | 0.8535 | 0.6554 | 0.3036 | 0.6607 | 0.4741 | 0.3261 |
| 0.3795 | 0.5570 | 0.8713 | 0.8353 | 0.7284 | 0.6254 | 0.8397 | 0.8170 | 0.9347 | 0.4787 |
| 0.9821 | 0.7411 | 0.5350 | 0.7501 | 0.2559 | 0.4101 | 0.8182 | 0.8124 | 0.8952 | 0.4668 |
| 0.2600 | 0.4862 | 0.7370 | 0.8037 | 0.3799 | 0.4819 | 0.3383 | 0.6648 | 0.3204 | 0.2512 |
| 0.3923 | 0.5655 | 0.7296 | 0.8004 | 0.8960 | 0.6700 | 0.9087 | 0.8246 | 0.8279 | 0.4515 |
| 0.6290 | 0.6592 | 0.2412 | 0.6241 | 0.8871 | 0.6670 | 0.0336 | 0.4937 | 0.3002 | 0.2411 |
| 0.6495 | 0.6641 | 0.3661 | 0.6705 | 0.7803 | 0.6366 | 0.0093 | 0.3368 | 0.5969 | 0.3679 |
| 0.6968 | 0.6662 | 0.5474 | 0.7501 | 0.0303 | 0.1756 | 0.3932 | 0.6735 | 0.1874 | 0.1828 |
| 0.7622 | 0.6915 | 0.4957 | 0.7261 | 0.8765 | 0.6657 | 0.6174 | 0.7573 | 0.0022 | 0.0099 |
| 0.9814 | 0.7411 | 0.9120 | 0.8444 | 0.9068 | 0.6736 | 0.2598 | 0.6561 | 0.0678 | 0.0891 |
| 0.1014 | 0.3659 | 0.0110 | 0.2328 | 0.1574 | 0.3277 | 0.1698 | 0.6229 | 0.6689 | 0.4004 |
| 0.9199 | 0.7281 | 0.6792 | 0.7857 | 0.0872 | 0.2629 | 0.0432 | 0.5116 | 0.0598 | 0.0827 |
| 0.3127 | 0.5330 | 0.5849 | 0.7622 | 0.8841 | 0.6670 | 0.1398 | 0.6069 | 0.5024 | 0.3355 |
| 0.9246 | 0.7286 | 0.7576 | 0.8135 | 0.3652 | 0.4812 | 0.6043 | 0.7510 | 0.0625 | 0.0847 |
| 0.2240 | 0.4709 | 0.0518 | 0.4026 | 0.4265 | 0.5082 | 0.0880 | 0.5648 | 0.0040 | 0.0141 |
| 0.4047 | 0.5715 | 0.7588 | 0.8135 | 0.8462 | 0.6538 | 0.6389 | 0.7665 | 0.0073 | 0.0194 |
| 0.6175 | 0.6579 | 0.1341 | 0.5071 | 0.0962 | 0.2751 | 0.2675 | 0.6561 | 0.7765 | 0.4360 |
| 0.8858 | 0.7219 | 0.9270 | 0.8444 | 0.0424 | 0.1982 | 0.7640 | 0.8016 | 0.0194 | 0.0395 |
| 0.0369 | 0.2961 | 0.0102 | 0.2244 | 0.0029 | 0.0549 | 0.9246 | 0.8246 | 0.1208 | 0.1344 |
| 0.9936 | 0.7431 | 0.4048 | 0.6950 | 0.5631 | 0.5718 | 0.1261 | 0.5944 | 0.3286 | 0.2545 |
| 0.9143 | 0.7281 | 0.7234 | 0.8004 | 0.0782 | 0.2550 | 0.0466 | 0.5116 | 0.0108 | 0.0264 |
| 0.4205 | 0.5828 | 0.0500 | 0.4026 | 0.1892 | 0.3581 | 0.3446 | 0.6648 | 0.4740 | 0.3261 |
| 0.7637 | 0.6915 | 0.8101 | 0.8177 | 0.0070 | 0.0766 | 0.0362 | 0.5116 | 0.0000 | 0.0007 |
| 0.3545 | 0.5538 | 0.2030 | 0.5977 | 0.3461 | 0.4742 | 0.4647 | 0.6953 | 0.8054 | 0.4446 |
| 0.3901 | 0.5655 | 0.7107 | 0.8004 | 0.7055 | 0.6200 | 0.1429 | 0.6069 | 0.0000 | 0.0006 |
| 0.4431 | 0.5982 | 0.3110 | 0.6550 | 0.0138 | 0.1128 | 0.9108 | 0.8246 | 0.0042 | 0.0141 |
| 0.1612 | 0.4185 | 0.0025 | 0.1565 | 0.6035 | 0.5870 | 0.5029 | 0.7056 | 0.0000 | 0.0006 |
| 0.5306 | 0.6262 | 0.9268 | 0.8444 | 0.7601 | 0.6341 | 0.3321 | 0.6648 | 0.0000 | 0.0000 |
| 0.3707 | 0.5570 | 0.6377 | 0.7782 | 0.8322 | 0.6518 | 0.6652 | 0.7801 | 0.1907 | 0.1841 |
| 0.2698 | 0.4963 | 0.4384 | 0.7007 | 0.1817 | 0.3581 | 0.5703 | 0.7388 | 0.1239 | 0.1366 |
| 0.8111 | 0.7058 | 0.6896 | 0.7890 | 0.2085 | 0.3713 | 0.6018 | 0.7506 | 0.0844 | 0.1041 |
| 0.7345 | 0.6810 | 0.8936 | 0.8427 | 0.9106 | 0.6736 | 0.9715 | 0.8347 | 0.0001 | 0.0010 |
| 0.5851 | 0.6479 | 0.2170 | 0.6056 | 0.6774 | 0.6152 | 0.2006 | 0.6258 | 0.1624 | 0.1673 |
| 0.3127 | 0.5330 | 0.0065 | 0.2084 | 0.1246 | 0.3083 | 0.4330 | 0.6821 | 0.1204 | 0.1344 |
| 0.6893 | 0.6642 | 0.2011 | 0.5977 | 0.5828 | 0.5786 | 0.7780 | 0.8048 | 0.8459 | 0.4577 |
| 0.3742 | 0.5570 | 0.2126 | 0.6009 | 0.1084 | 0.2946 | 0.7754 | 0.8048 | 0.5304 | 0.3439 |
| 0.5493 | 0.6273 | 0.5796 | 0.7622 | 0.4153 | 0.4992 | 0.9203 | 0.8246 | 0.2185 | 0.2015 |
| 0.5048 | 0.6218 | 0.8093 | 0.8177 | 0.6146 | 0.5925 | 0.2672 | 0.6561 | 0.0026 | 0.0111 |
| 0.6435 | 0.6641 | 0.6457 | 0.7782 | 0.8343 | 0.6518 | 0.8699 | 0.8201 | 0.0660 | 0.0879 |
| 0.5307 | 0.6262 | 0.9593 | 0.8476 | 0.0292 | 0.1728 | 0.9564 | 0.8312 | 0.0122 | 0.0291 |
| 0.7738 | 0.6952 | 0.5406 | 0.7501 | 0.4276 | 0.5084 | 0.6143 | 0.7548 | 0.7209 | 0.4176 |
| 0.5656 | 0.6393 | 0.7164 | 0.8004 | 0.7760 | 0.6352 | 0.6502 | 0.7764 | 0.6282 | 0.3810 |
| 0.0400 | 0.3026 | 0.4015 | 0.6946 | 0.8528 | 0.6554 | 0.8665 | 0.8201 | 0.0266 | 0.0489 |
| 0.1823 | 0.4361 | 0.3523 | 0.6627 | 0.0406 | 0.1928 | 0.3646 | 0.6648 | 0.3466 | 0.2653 |
| 0.1768 | 0.4322 | 0.0430 | 0.3767 | 0.0303 | 0.1756 | 0.0989 | 0.5714 | 0.0156 | 0.0342 |
| 0.7118 | 0.6724 | 0.1019 | 0.4999 | 0.0589 | 0.2308 | 0.3854 | 0.6693 | 0.2832 | 0.2328 |
| 0.4441 | 0.5983 | 0.8549 | 0.8302 | 0.2544 | 0.4095 | 0.2202 | 0.6280 | 0.9451 | 0.4815 |
| 0.7033 | 0.6696 | 0.2289 | 0.6110 | 0.3186 | 0.4548 | 0.7607 | 0.8010 | 0.0015 | 0.0076 |
| 0.4342 | 0.5933 | 0.1789 | 0.5787 | 0.5941 | 0.5837 | 0.9564 | 0.8312 | 0.8018 | 0.4441 |
| 0.9496 | 0.7359 | 0.1771 | 0.5752 | 0.4330 | 0.5107 | 0.3446 | 0.6648 | 0.2388 | 0.2129 |
| 0.8517 | 0.7193 | 0.2553 | 0.6326 | 0.3884 | 0.4870 | 0.2100 | 0.6280 | 0.7467 | 0.4272 |
| 0.6875 | 0.6642 | 0.6901 | 0.7890 | 0.6868 | 0.6165 | 0.4798 | 0.7017 | 0.0000 | 0.0000 |
| 0.0115 | 0.2237 | 0.5698 | 0.7622 | 0.9088 | 0.6736 | 0.2371 | 0.6397 | 0.8939 | 0.4668 |
| 0.5430 | 0.6273 | 0.5408 | 0.7501 | 0.2663 | 0.4184 | 0.4517 | 0.6869 | 0.0000 | 0.0000 |
| | | | | | | 0.3559 | 0.6648 | 0.0128 | 0.0295 |
| | | | | | | | | 0.0387 | 0.0615 |
| | | | | | | | | 0.0272 | 0.0494 |
| 0.7611 | 0.6915 | 0.1957 | 0.5977 | 0.0046 | 0.0647 | 0.3200 | 0.6635 | 0.1213 | 0.1344 |
| 0.3756 | 0.5570 | 0.3372 | 0.6550 | 0.4466 | 0.5191 | 0.6468 | 0.7736 | 0.0450 | 0.0692 |
| 0.3053 | 0.5297 | 0.8917 | 0.8427 | 0.5438 | 0.5632 | 0.2298 | 0.6375 | 0.0001 | 0.0013 |
| 0.6021 | 0.6554 | 0.1154 | 0.5031 | 0.0207 | 0.1450 | 0.7977 | 0.8058 | 0.5188 | 0.3390 |
| 0.4067 | 0.5723 | 0.9151 | 0.8444 | 0.3272 | 0.4638 | 0.5910 | 0.7484 | 0.0603 | 0.0831 |
| 0.4465 | 0.5992 | 0.7095 | 0.8004 | 0.0889 | 0.2629 | 0.4320 | 0.6821 | 0.5957 | 0.3679 |
| 0.0074 | 0.2237 | 0.7607 | 0.8135 | 0.2260 | 0.3869 | 0.8516 | 0.8177 | 0.1440 | 0.1532 |
| 0.3709 | 0.5570 | 0.2880 | 0.6500 | 0.0127 | 0.1101 | 0.4975 | 0.7056 | 0.8825 | 0.4647 |
| 0.0215 | 0.2497 | 0.0474 | 0.3955 | 0.4756 | 0.5265 | 0.0667 | 0.5225 | 0.0051 | 0.0160 |
| 0.3009 | 0.5249 | 0.8474 | 0.8286 | 0.6431 | 0.6046 | 0.1577 | 0.6131 | 0.0014 | 0.0072 |
| 0.0274 | 0.2533 | 0.0092 | 0.2084 | 0.0014 | 0.0375 | 0.5555 | 0.7345 | 0.2638 | 0.2219 |
| 0.0698 | 0.3270 | 0.2285 | 0.6110 | 0.5823 | 0.5786 | 0.8448 | 0.8170 | 0.0006 | 0.0046 |
| 0.0858 | 0.3518 | 0.4636 | 0.7101 | 0.4312 | 0.5107 | 0.3296 | 0.6648 | 0.0002 | 0.0021 |
| 0.1348 | 0.4035 | 0.8472 | 0.8286 | 0.2726 | 0.4242 | 0.1932 | 0.6258 | 0.1695 | 0.1716 |
| 0.2647 | 0.4921 | 0.1630 | 0.5502 | 0.5683 | 0.5733 | 0.4972 | 0.7056 | 0.9626 | 0.4866 |
| 0.0141 | 0.2237 | 0.0547 | 0.4144 | 0.6226 | 0.5957 | 0.5465 | 0.7328 | 0.5702 | 0.3592 |
| 0.3068 | 0.5309 | 0.1183 | 0.5031 | 0.8875 | 0.6670 | 0.9642 | 0.8347 | 0.7774 | 0.4360 |
| 0.1599 | 0.4185 | 0.4890 | 0.7234 | 0.0041 | 0.0591 | 0.2381 | 0.6397 | 0.3426 | 0.2632 |
| 0.0026 | 0.1976 | 0.2368 | 0.6235 | 0.1124 | 0.2946 | 0.7933 | 0.8058 | 0.0579 | 0.0808 |
| 0.9952 | 0.7431 | 0.5368 | 0.7501 | 0.3501 | 0.4770 | 0.8218 | 0.8133 | 0.0376 | 0.0612 |
| 0.4802 | 0.6122 | 0.0173 | 0.2839 | 0.0367 | 0.1918 | 0.7052 | 0.7879 | 0.9163 | 0.4742 |
| 0.0611 | 0.3199 | 0.8043 | 0.8177 | 0.8149 | 0.6465 | 0.9097 | 0.8246 | 0.8808 | 0.4643 |
| 0.0578 | 0.3182 | 0.9974 | 0.8533 | 0.3704 | 0.4815 | 0.5291 | 0.7209 | 0.0059 | 0.0171 |
| 0.1785 | 0.4322 | 0.8615 | 0.8326 | 0.2057 | 0.3713 | 0.3634 | 0.6648 | 0.0145 | 0.0324 |
| 0.0917 | 0.3547 | 0.0313 | 0.3457 | 0.0009 | 0.0358 | 0.1296 | 0.6029 | 0.9443 | 0.4815 |
| 0.0087 | 0.2237 | 0.8676 | 0.8353 | 0.2664 | 0.4184 | 0.8918 | 0.8246 | 0.0298 | 0.0518 |
| 0.3536 | 0.5537 | 0.8844 | 0.8410 | 0.8014 | 0.6445 | 0.3383 | 0.6648 | 0.2316 | 0.2105 |
| 0.2436 | 0.4738 | 0.5501 | 0.7501 | 0.5217 | 0.5517 | 0.0218 | 0.3996 | 0.4601 | 0.3224 |
| 0.4883 | 0.6173 | 0.0191 | 0.2914 | 0.9167 | 0.6736 | 0.0856 | 0.5648 | 0.5625 | 0.3572 |
| 0.0774 | 0.3417 | 0.1267 | 0.5071 | 0.9085 | 0.6736 | 0.0013 | 0.2465 | 0.2976 | 0.2403 |
| 0.9093 | 0.7281 | 0.2585 | 0.6326 | 0.1963 | 0.3624 | 0.0070 | 0.3316 | 0.2536 | 0.2188 |
| 0.1080 | 0.3668 | 0.0046 | 0.1882 | 0.5174 | 0.5510 | 0.0167 | 0.3996 | 0.3069 | 0.2448 |
| 0.4328 | 0.5925 | 0.4360 | 0.7007 | 0.3649 | 0.4812 | 0.0326 | 0.4878 | 0.9280 | 0.4787 |
| 0.1649 | 0.4260 | 0.5629 | 0.7608 | 0.0023 | 0.0498 | 0.1649 | 0.6172 | 0.4726 | 0.3261 |
| 0.2187 | 0.4709 | 0.3222 | 0.6550 | 0.0825 | 0.2629 | 0.0761 | 0.5375 | 0.4783 | 0.3275 |
| 0.9711 | 0.7390 | 0.8396 | 0.8280 | 0.8438 | 0.6536 | 0.2732 | 0.6595 | 0.9086 | 0.4719 |
| 0.1483 | 0.4179 | 0.6225 | 0.7782 | 0.6701 | 0.6152 | 0.9130 | 0.8246 | 0.0008 | 0.0051 |
| 0.7861 | 0.6966 | 0.7922 | 0.8177 | 0.9270 | 0.6736 | 0.3263 | 0.6640 | 0.6853 | 0.4056 |
| 0.0625 | 0.3199 | 0.4333 | 0.7007 | 0.9099 | 0.6736 | 0.8334 | 0.8170 | 0.4110 | 0.2971 |
| 0.7299 | 0.6795 | 0.3212 | 0.6550 | 0.6614 | 0.6129 | 0.0312 | 0.4825 | 0.0616 | 0.0843 |
| 0.5257 | 0.6262 | 0.0641 | 0.4455 | 0.1376 | 0.3151 | 0.4622 | 0.6929 | 0.2418 | 0.2136 |
| 0.8389 | 0.7175 | 0.5259 | 0.7446 | 0.4029 | 0.4960 | 0.7244 | 0.7918 | 0.6146 | 0.3757 |
| 0.2306 | 0.4709 | 0.1067 | 0.4999 | 0.1299 | 0.3130 | 0.4247 | 0.6821 | 0.5265 | 0.3436 |
| 0.4616 | 0.6004 | 0.0609 | 0.4455 | 0.1356 | 0.3151 | 0.7692 | 0.8025 | 0.0146 | 0.0325 |
| 0.6096 | 0.6579 | 0.0496 | 0.4026 | 0.0877 | 0.2629 | 0.0807 | 0.5451 | 0.0373 | 0.0612 |
| 0.6870 | 0.6642 | 0.4395 | 0.7007 | 0.4186 | 0.5015 | 0.2835 | 0.6595 | 0.8784 | 0.4643 |
| 0.0218 | 0.2497 | 0.4413 | 0.7020 | 0.7415 | 0.6307 | 0.3999 | 0.6735 | 0.0019 | 0.0090 |
| 0.0595 | 0.3182 | 0.6889 | 0.7890 | 0.1066 | 0.2930 | 0.1989 | 0.6258 | 0.0281 | 0.0502 |
| 0.4122 | 0.5760 | 0.7055 | 0.8004 | 0.4144 | 0.4992 | 0.6697 | 0.7805 | 0.7713 | 0.4347 |
| 0.4768 | 0.6122 | 0.7700 | 0.8147 | 0.8438 | 0.6536 | 0.5972 | 0.7506 | 0.4451 | 0.3153 |
| 0.2164 | 0.4709 | 0.2963 | 0.6533 | 0.6023 | 0.5870 | 0.5178 | 0.7106 | 0.9900 | 0.4951 |
| 0.2326 | 0.4709 | 0.0192 | 0.2914 | 0.0881 | 0.2629 | 0.1409 | 0.6069 | 0.0066 | 0.0181 |
| 0.8947 | 0.7230 | 0.0514 | 0.4026 | 0.7041 | 0.6196 | 0.4247 | 0.6821 | 0.0099 | 0.0250 |
| 0.1940 | 0.4446 | 0.6767 | 0.7856 | 0.8428 | 0.6536 | 0.6869 | 0.7844 | 0.1617 | 0.1670 |
| 0.1364 | 0.4035 | 0.2166 | 0.6056 | 0.6339 | 0.5991 | 0.9990 | 0.8423 | 0.5680 | 0.3584 |
| 0.2289 | 0.4709 | 0.3033 | 0.6550 | 0.4713 | 0.5264 | 0.2572 | 0.6561 | 0.4808 | 0.3278 |
| 0.8836 | 0.7219 | 0.6166 | 0.7764 | 0.9601 | 0.6782 | 0.0654 | 0.5225 | 0.2592 | 0.2198 |
| 0.6910 | 0.6644 | 0.0061 | 0.2084 | 0.0760 | 0.2530 | 0.0917 | 0.5648 | 0.0000 | 0.0004 |
| 0.3256 | 0.5421 | 0.3075 | 0.6550 | 0.1309 | 0.3131 | 0.1928 | 0.6258 | 0.0871 | 0.1049 |
| 0.0199 | 0.2497 | 0.0374 | 0.3757 | 0.3568 | 0.4796 | 0.2188 | 0.6280 | 0.5810 | 0.3621 |
| 0.2843 | 0.5094 | 0.8454 | 0.8286 | 0.0703 | 0.2448 | 0.7390 | 0.7941 | 0.0049 | 0.0156 |
| 0.1560 | 0.4185 | 0.2634 | 0.6395 | 0.0048 | 0.0656 | 0.3016 | 0.6607 | 0.9127 | 0.4729 |
| 0.2890 | 0.5133 | 0.3436 | 0.6614 | 0.0063 | 0.0739 | 0.2032 | 0.6258 | 0.5643 | 0.3575 |
| 0.6506 | 0.6641 | 0.5699 | 0.7622 | 0.5041 | 0.5447 | 0.7455 | 0.7941 | 0.1475 | 0.1559 |
| 0.5141 | 0.6257 | 0.1235 | 0.5031 | 0.1745 | 0.3524 | 0.3732 | 0.6648 | 0.0000 | 0.0002 |
| 0.7975 | 0.7024 | 0.4312 | 0.7007 | 0.1205 | 0.3046 | 0.3554 | 0.6648 | 0.4734 | 0.3261 |
| 0.6853 | 0.6642 | 0.1970 | 0.5977 | 0.6981 | 0.6188 | 0.8933 | 0.8246 | 0.0071 | 0.0193 |
| 0.0435 | 0.3026 | 0.0235 | 0.2968 | 0.0310 | 0.1784 | 0.4478 | 0.6869 | 0.5581 | 0.3561 |
| 0.8886 | 0.7227 | 0.3687 | 0.6705 | 0.2821 | 0.4322 | 0.2003 | 0.6258 | 0.2561 | 0.2192 |
| 0.3388 | 0.5484 | 0.9235 | 0.8444 | 0.8152 | 0.6465 | 0.4406 | 0.6869 | 0.0000 | 0.0002 |
| 0.6689 | 0.6642 | 0.1252 | 0.5043 | 0.8114 | 0.6465 | 0.4846 | 0.7017 | 0.0156 | 0.0342 |
| 0.0119 | 0.2237 | 0.4648 | 0.7101 | 0.9407 | 0.6736 | 0.6267 | 0.7630 | 0.6188 | 0.3773 |
| 0.0752 | 0.3417 | 0.0009 | 0.1535 | 0.2086 | 0.3713 | 0.2367 | 0.6397 | 0.2847 | 0.2328 |
| 0.0791 | 0.3430 | 0.1490 | 0.5300 | 0.2290 | 0.3899 | 0.3955 | 0.6735 | 0.5874 | 0.3645 |
| 0.0257 | 0.2518 | 0.6090 | 0.7706 | 0.4529 | 0.5216 | 0.9075 | 0.8246 | 0.0449 | 0.0692 |
| 0.9525 | 0.7367 | 0.5251 | 0.7446 | 0.9917 | 0.6909 | 0.3695 | 0.6648 | 0.1582 | 0.1645 |
| 0.5749 | 0.6410 | 0.5890 | 0.7627 | 0.9957 | 0.6909 | 0.5090 | 0.7061 | 0.3763 | 0.2801 |
| 0.8435 | 0.7175 | 0.2821 | 0.6490 | 0.7898 | 0.6398 | 0.9374 | 0.8269 | 0.0832 | 0.1036 |
| 0.7643 | 0.6915 | 0.6077 | 0.7706 | 0.7933 | 0.6402 | 0.0703 | 0.5225 | 0.0839 | 0.1038 |
| 0.2696 | 0.4963 | 0.0682 | 0.4509 | 0.0600 | 0.2308 | 0.4939 | 0.7056 | 0.1433 | 0.1528 |
| 0.9785 | 0.7411 | 0.9025 | 0.8444 | 0.5490 | 0.5654 | 0.6628 | 0.7801 | 0.8852 | 0.4655 |
| 0.4515 | 0.5999 | 0.0056 | 0.2084 | 0.1345 | 0.3151 | 0.0234 | 0.4066 | 0.5077 | 0.3370 |
| 0.8860 | 0.7219 | 0.7681 | 0.8147 | 0.8674 | 0.6620 | 0.2170 | 0.6280 | 0.0109 | 0.0264 |
| 0.0078 | 0.2237 | 0.0176 | 0.2839 | 0.0613 | 0.2329 | 0.3649 | 0.6648 | 0.3637 | 0.2742 |
| 0.1588 | 0.4185 | 0.9494 | 0.8469 | 0.4153 | 0.4992 | 0.6843 | 0.7844 | 0.0820 | 0.1026 |
| 0.0582 | 0.3182 | 0.0859 | 0.4917 | 0.1889 | 0.3581 | 0.4787 | 0.7017 | 0.4928 | 0.3306 |
| 0.2395 | 0.4709 | 0.4022 | 0.6946 | 0.5798 | 0.5786 | 0.3759 | 0.6648 | 0.0406 | 0.0639 |
| 0.5230 | 0.6260 | 0.5474 | 0.7501 | 0.5729 | 0.5762 | 0.8685 | 0.8201 | 0.0549 | 0.0774 |
| 0.0884 | 0.3523 | 0.1953 | 0.5977 | 0.1419 | 0.3151 | 0.8032 | 0.8058 | 0.3642 | 0.2742 |
| 0.1689 | 0.4260 | 0.6316 | 0.7782 | 0.8192 | 0.6467 | 0.7472 | 0.7941 | 0.0756 | 0.0969 |
| 0.9140 | 0.7281 | 0.5529 | 0.7526 | 0.7726 | 0.6352 | 0.7030 | 0.7879 | 0.5631 | 0.3572 |
| 0.1512 | 0.4183 | 0.2065 | 0.5977 | 0.2660 | 0.4184 | 0.4025 | 0.6735 | 0.1387 | 0.1497 |
| 0.4371 | 0.5949 | 0.3307 | 0.6550 | 0.7685 | 0.6352 | 0.9176 | 0.8246 | 0.2438 | 0.2148 |
| 0.9647 | 0.7367 | 0.1754 | 0.5741 | 0.6335 | 0.5991 | 0.4987 | 0.7056 | 0.0683 | 0.0891 |
| 0.6165 | 0.6579 | 0.8895 | 0.8427 | 0.1891 | 0.3581 | 0.8101 | 0.8090 | 0.0000 | 0.0002 |
| 0.0998 | 0.3640 | 0.0173 | 0.2839 | 0.4325 | 0.5107 | 0.2922 | 0.6600 | 0.1349 | 0.1463 |
| 0.0265 | 0.2518 | 0.3690 | 0.6705 | 0.4663 | 0.5264 | 0.7828 | 0.8048 | 0.2852 | 0.2328 |
| 0.2540 | 0.4788 | 0.4332 | 0.7007 | 0.9395 | 0.6736 | 0.4784 | 0.7017 | 0.0283 | 0.0503 |
| 0.0423 | 0.3026 | 0.1382 | 0.5119 | 0.3583 | 0.4796 | 0.8707 | 0.8201 | 0.3301 | 0.2552 |
| 0.4580 | 0.6004 | 0.1336 | 0.5071 | 0.0896 | 0.2629 | 0.4325 | 0.6821 | 0.9404 | 0.4807 |
| 0.8313 | 0.7149 | 0.7570 | 0.8135 | 0.8704 | 0.6634 | 0.3774 | 0.6649 | 0.4174 | 0.2998 |
| 0.6203 | 0.6579 | 0.1525 | 0.5361 | 0.3409 | 0.4742 | 0.3043 | 0.6607 | 0.0024 | 0.0104 |
| 0.0547 | 0.3182 | 0.2552 | 0.6326 | 0.6813 | 0.6153 | 0.6559 | 0.7795 | 0.0500 | 0.0747 |
| 0.4512 | 0.5999 | 0.5456 | 0.7501 | 0.1459 | 0.3167 | 0.7068 | 0.7879 | 0.5381 | 0.3462 |
| 0.1957 | 0.4463 | 0.9479 | 0.8469 | 0.4590 | 0.5239 | 0.0148 | 0.3753 | 0.3066 | 0.2448 |
| 0.3711 | 0.5570 | 0.1613 | 0.5502 | 0.3596 | 0.4796 | 0.1624 | 0.6166 | 0.1684 | 0.1712 |
| 0.3971 | 0.5681 | 0.3605 | 0.6684 | 0.1960 | 0.3624 | 0.0385 | 0.5116 | 0.2167 | 0.2002 |
| 0.0250 | 0.2518 | 0.0032 | 0.1565 | 0.0060 | 0.0737 | 0.4607 | 0.6929 | 0.0540 | 0.0771 |
| 0.5028 | 0.6204 | 0.9756 | 0.8504 | 0.4107 | 0.4992 | 0.3225 | 0.6635 | 0.2629 | 0.2219 |
| 0.4010 | 0.5708 | 0.4713 | 0.7161 | 0.9484 | 0.6745 | 0.9145 | 0.8246 | 0.0018 | 0.0089 |
| 0.0960 | 0.3622 | 0.3346 | 0.6550 | 0.1443 | 0.3151 | 0.2556 | 0.6561 | 0.0484 | 0.0735 |
| 0.6128 | 0.6579 | 0.3366 | 0.6550 | 0.2382 | 0.3980 | 0.5024 | 0.7056 | 0.0009 | 0.0056 |
| 0.1573 | 0.4185 | 0.3110 | 0.6550 | 0.0836 | 0.2629 | 0.1212 | 0.5944 | 0.1141 | 0.1296 |
| 0.0134 | 0.2237 | 0.1243 | 0.5031 | 0.0323 | 0.1796 | 0.1066 | 0.5768 | 0.5440 | 0.3490 |
| 0.0412 | 0.3026 | 0.1314 | 0.5071 | 0.0689 | 0.2439 | 0.7428 | 0.7941 | 0.0293 | 0.0513 |
| 0.2349 | 0.4709 | 0.0906 | 0.4917 | 0.0262 | 0.1623 | 0.0468 | 0.5116 | 0.4392 | 0.3116 |
| 0.0599 | 0.3182 | 0.8916 | 0.8427 | 0.6607 | 0.6129 | 0.7241 | 0.7918 | 0.2721 | 0.2274 |
| 0.0379 | 0.2966 | 0.8347 | 0.8269 | 0.1272 | 0.3101 | 0.1359 | 0.6069 | 0.2569 | 0.2193 |
| 0.9413 | 0.7345 | 0.3324 | 0.6550 | 0.3012 | 0.4441 | 0.1947 | 0.6258 | 0.2498 | 0.2179 |
| 0.0576 | 0.3182 | 0.8605 | 0.8326 | 0.5606 | 0.5711 | 0.5030 | 0.7056 | 0.0234 | 0.0444 |
| 0.1172 | 0.3748 | 0.4852 | 0.7227 | 0.7731 | 0.6352 | 0.1556 | 0.6131 | 0.0297 | 0.0518 |
| 0.2823 | 0.5081 | 0.0866 | 0.4917 | 0.0040 | 0.0591 | 0.0055 | 0.2791 | 0.0057 | 0.0168 |
| 0.3929 | 0.5655 | 0.4119 | 0.6999 | 0.7768 | 0.6352 | 0.6021 | 0.7506 | 0.0007 | 0.0046 |
| 0.5407 | 0.6273 | 0.1153 | 0.5031 | 0.1809 | 0.3581 | 0.8526 | 0.8177 | 0.0001 | 0.0013 |
| 0.7682 | 0.6924 | 0.3510 | 0.6627 | 0.0091 | 0.0921 | 0.0297 | 0.4724 | 0.0002 | 0.0021 |
| 0.3973 | 0.5681 | 0.5089 | 0.7340 | 0.2051 | 0.3713 | 0.3076 | 0.6635 | 0.2066 | 0.1931 |
| 0.3376 | 0.5484 | 0.2807 | 0.6478 | 0.3673 | 0.4812 | 0.0504 | 0.5116 | 0.4632 | 0.3236 |
| 0.8773 | 0.7219 | 0.9819 | 0.8530 | 0.6246 | 0.5957 | 0.1235 | 0.5944 | 0.2340 | 0.2112 |
| 0.4256 | 0.5863 | 0.9961 | 0.8533 | 0.4696 | 0.5264 | 0.8669 | 0.8201 | 0.8274 | 0.4515 |
| 0.2484 | 0.4741 | 0.9137 | 0.8444 | 0.2745 | 0.4248 | 0.3698 | 0.6648 | 0.2032 | 0.1914 |
| 0.0018 | 0.1952 | 0.0276 | 0.3156 | 0.2172 | 0.3760 | 0.0005 | 0.2465 | 0.0014 | 0.0072 |
| 0.3381 | 0.5484 | 0.9637 | 0.8486 | 0.2116 | 0.3734 | 0.1621 | 0.6166 | 0.0057 | 0.0168 |
| 0.6942 | 0.6653 | 0.6635 | 0.7800 | 0.4860 | 0.5333 | 0.0581 | 0.5136 | 0.5709 | 0.3592 |
| 0.0176 | 0.2407 | 0.5715 | 0.7622 | 0.4266 | 0.5082 | 0.9099 | 0.8246 | 0.0104 | 0.0258 |
| 0.5174 | 0.6257 | 0.2882 | 0.6500 | 0.2797 | 0.4296 | 0.6638 | 0.7801 | 0.6796 | 0.4040 |
| 0.9682 | 0.7383 | 0.2047 | 0.5977 | 0.4753 | 0.5265 | 0.4243 | 0.6821 | 0.2138 | 0.1983 |
| 0.2630 | 0.4903 | 0.3368 | 0.6550 | 0.2477 | 0.4052 | 0.4018 | 0.6735 | 0.1353 | 0.1464 |
| 0.6774 | 0.6642 | 0.6659 | 0.7800 | 0.7418 | 0.6307 | 0.6119 | 0.7543 | 0.0042 | 0.0141 |
| 0.6007 | 0.6551 | 0.0414 | 0.3763 | 0.7252 | 0.6254 | 0.3863 | 0.6693 | 0.0001 | 0.0010 |
| 0.2445 | 0.4740 | 0.9052 | 0.8444 | 0.2956 | 0.4402 | 0.1916 | 0.6258 | 0.2649 | 0.2222 |
| 0.9529 | 0.7367 | 0.3120 | 0.6550 | 0.9236 | 0.6736 | 0.0947 | 0.5648 | 0.0029 | 0.0117 |
| 0.5913 | 0.6522 | 0.2207 | 0.6071 | 0.8166 | 0.6465 | 0.1759 | 0.6258 | 0.0001 | 0.0013 |
| 0.4035 | 0.5710 | 0.1167 | 0.5031 | 0.6025 | 0.5870 | 0.1331 | 0.6069 | 0.8147 | 0.4478 |
| 0.1608 | 0.4185 | 0.3827 | 0.6811 | 0.0003 | 0.0255 | 0.3801 | 0.6652 | 0.8047 | 0.4446 |
| 0.8558 | 0.7193 | 0.8727 | 0.8353 | 0.0199 | 0.1425 | 0.8033 | 0.8058 | 0.7579 | 0.4315 |
| 0.4968 | 0.6198 | 0.5448 | 0.7501 | 0.0019 | 0.0485 | 0.4422 | 0.6869 | 0.7949 | 0.4426 |
| 0.8918 | 0.7230 | 0.6422 | 0.7782 | 0.0033 | 0.0549 | 0.9835 | 0.8347 | 0.8968 | 0.4668 |
| 0.7384 | 0.6827 | 0.5278 | 0.7446 | 0.0135 | 0.1128 | 0.5134 | 0.7071 | 0.5591 | 0.3561 |
| 0.9120 | 0.7281 | 0.5491 | 0.7501 | 0.0572 | 0.2296 | 0.9114 | 0.8246 | 0.5185 | 0.3390 |
| 0.0553 | 0.3182 | 0.1494 | 0.5300 | 0.2611 | 0.4138 | 0.1719 | 0.6245 | 0.3203 | 0.2512 |
| 0.6949 | 0.6653 | 0.5117 | 0.7340 | 0.3440 | 0.4742 | 0.2615 | 0.6561 | 0.9438 | 0.4815 |
| 0.5487 | 0.6273 | 0.2768 | 0.6478 | 0.6443 | 0.6046 | 0.3500 | 0.6648 | 0.8722 | 0.4630 |
| 0.5491 | 0.6273 | 0.8550 | 0.8302 | 0.6767 | 0.6152 | 0.1040 | 0.5714 | 0.2608 | 0.2208 |
| 0.7455 | 0.6849 | 0.4695 | 0.7159 | 0.6270 | 0.5966 | 0.5888 | 0.7484 | 0.0051 | 0.0159 |
| 0.8766 | 0.7219 | 0.0070 | 0.2084 | 0.7759 | 0.6352 | 0.5848 | 0.7462 | 0.2276 | 0.2081 |
| 0.5585 | 0.6336 | 0.3776 | 0.6774 | 0.7039 | 0.6196 | 0.2116 | 0.6280 | 0.0021 | 0.0098 |
| 0.6898 | 0.6642 | 0.9569 | 0.8469 | 0.0693 | 0.2439 | 0.1350 | 0.6069 | 0.8220 | 0.4505 |
| 0.0445 | 0.3046 | 0.7102 | 0.8004 | 0.2718 | 0.4242 | 0.4525 | 0.6869 | 0.0066 | 0.0181 |
| 0.1146 | 0.3701 | 0.6532 | 0.7782 | 0.1511 | 0.3199 | 0.9393 | 0.8275 | 0.0003 | 0.0028 |
| 0.3312 | 0.5439 | 0.0891 | 0.4917 | 0.9390 | 0.6736 | 0.0414 | 0.5116 | 0.1079 | 0.1236 |
| 0.3924 | 0.5655 | 0.7600 | 0.8135 | 0.9261 | 0.6736 | 0.4486 | 0.6869 | 0.0647 | 0.0867 |
| 0.7830 | 0.6954 | 0.8994 | 0.8441 | 0.1094 | 0.2946 | 0.2807 | 0.6595 | 0.4739 | 0.3261 |
| 0.2407 | 0.4709 | 0.0301 | 0.3374 | 0.4537 | 0.5216 | 0.2204 | 0.6280 | 0.9546 | 0.4841 |
| 0.4557 | 0.6004 | 0.7184 | 0.8004 | 0.1281 | 0.3111 | 0.4286 | 0.6821 | 0.5927 | 0.3668 |
| 0.0205 | 0.2497 | 0.7145 | 0.8004 | 0.0913 | 0.2637 | 0.9264 | 0.8246 | 0.0001 | 0.0012 |
| 0.1213 | 0.3802 | 0.3905 | 0.6899 | 0.1749 | 0.3524 | 0.2872 | 0.6600 | 0.0042 | 0.0141 |
| 0.5374 | 0.6273 | 0.3754 | 0.6750 | 0.7230 | 0.6254 | 0.3048 | 0.6607 | 0.1343 | 0.1460 |
| 0.6990 | 0.6674 | 0.8675 | 0.8353 | 0.3149 | 0.4505 | 0.2588 | 0.6561 | 0.2575 | 0.2193 |
| 0.4997 | 0.6198 | 0.3034 | 0.6550 | 0.0642 | 0.2359 | 0.0957 | 0.5660 | 0.6117 | 0.3754 |
| 0.7824 | 0.6954 | 0.2376 | 0.6235 | 0.0928 | 0.2667 | 0.9153 | 0.8246 | 0.4771 | 0.3275 |
| 0.1438 | 0.4142 | 0.4372 | 0.7007 | 0.0099 | 0.0944 | 0.1496 | 0.6131 | 0.6396 | 0.3869 |
| 0.4716 | 0.6075 | 0.2892 | 0.6500 | 0.2475 | 0.4052 | 0.0935 | 0.5648 | 0.9120 | 0.4729 |
| 0.7376 | 0.6827 | 0.9053 | 0.8444 | 0.5653 | 0.5730 | 0.4023 | 0.6735 | 0.0292 | 0.0513 |
| 0.0488 | 0.3055 | 0.6538 | 0.7782 | 0.6919 | 0.6181 | 0.1012 | 0.5714 | 0.7347 | 0.4235 |
| 0.9584 | 0.7367 | 0.9357 | 0.8447 | 0.4725 | 0.5264 | 0.3177 | 0.6635 | 0.4571 | 0.3218 |
| 0.2925 | 0.5154 | 0.9124 | 0.8444 | 0.8973 | 0.6701 | 0.5088 | 0.7061 | 0.3786 | 0.2808 |
| 0.8731 | 0.7219 | 0.9753 | 0.8504 | 0.5348 | 0.5566 | 0.9701 | 0.8347 | 0.0066 | 0.0181 |
| 0.7548 | 0.6904 | 0.6652 | 0.7800 | 0.1821 | 0.3581 | 0.5628 | 0.7370 | 0.2402 | 0.2129 |
| 0.2157 | 0.4709 | 0.8364 | 0.8269 | 0.2768 | 0.4261 | 0.3466 | 0.6648 | 0.0005 | 0.0037 |
| 0.4544 | 0.6004 | 0.0992 | 0.4999 | 0.1427 | 0.3151 | 0.6534 | 0.7778 | 0.0001 | 0.0008 |
| 0.2188 | 0.4709 | 0.2019 | 0.5977 | 0.6753 | 0.6152 | 0.2026 | 0.6258 | 0.2059 | 0.1930 |
| 0.0472 | 0.3055 | 0.2970 | 0.6533 | 0.7036 | 0.6196 | 0.1039 | 0.5714 | 0.0053 | 0.0162 |
| 0.2171 | 0.4709 | 0.9234 | 0.8444 | 0.0703 | 0.2448 | 0.7084 | 0.7879 | 0.0233 | 0.0444 |
| 0.2399 | 0.4709 | 0.0891 | 0.4917 | 0.5683 | 0.5733 | 0.0359 | 0.5116 | 0.2396 | 0.2129 |
| 0.2498 | 0.4750 | 0.7167 | 0.8004 | 0.0098 | 0.0944 | 0.2924 | 0.6600 | 0.6824 | 0.4052 |
| 0.0557 | 0.3182 | 0.1031 | 0.4999 | 0.2063 | 0.3713 | 0.4519 | 0.6869 | 0.7014 | 0.4127 |
| 0.4450 | 0.5983 | 0.5779 | 0.7622 | 0.9421 | 0.6736 | 0.7079 | 0.7879 | 0.7426 | 0.4272 |
| 0.8955 | 0.7230 | 0.4644 | 0.7101 | 0.4705 | 0.5264 | 0.2173 | 0.6280 | 0.8024 | 0.4441 |
| 0.1494 | 0.4179 | 0.4861 | 0.7227 | 0.9247 | 0.6736 | 0.9762 | 0.8347 | 0.7631 | 0.4328 |
| 0.6373 | 0.6641 | 0.4850 | 0.7227 | 0.3451 | 0.4742 | 0.4900 | 0.7055 | 0.3169 | 0.2497 |
| 0.6457 | 0.6641 | 0.2582 | 0.6326 | 0.0453 | 0.2035 | 0.3642 | 0.6648 | 0.4851 | 0.3292 |
| 0.0572 | 0.3182 | 0.0246 | 0.2968 | 0.5531 | 0.5673 | 0.0044 | 0.2605 | 0.0004 | 0.0029 |
| 0.2358 | 0.4709 | 0.3334 | 0.6550 | 0.0644 | 0.2359 | 0.1243 | 0.5944 | 0.7883 | 0.4405 |
| 0.6067 | 0.6579 | 0.4279 | 0.7007 | 0.1504 | 0.3199 | 0.1743 | 0.6256 | 0.9350 | 0.4787 |
| 0.8388 | 0.7175 | 0.6371 | 0.7782 | 0.3671 | 0.4812 | 0.4835 | 0.7017 | 0.8582 | 0.4604 |
| 0.2418 | 0.4717 | 0.6542 | 0.7782 | 0.0035 | 0.0562 | 0.8717 | 0.8201 | 0.9909 | 0.4951 |
| 0.0979 | 0.3626 | 0.2379 | 0.6235 | 0.0799 | 0.2581 | 0.0116 | 0.3368 | 0.0143 | 0.0322 |
| 0.4397 | 0.5967 | 0.3108 | 0.6550 | 0.0167 | 0.1257 | 0.0567 | 0.5136 | 0.0137 | 0.0312 |
| 0.0919 | 0.3547 | 0.2736 | 0.6478 | 0.1265 | 0.3097 | 0.6710 | 0.7805 | 0.0860 | 0.1047 |
| 0.3562 | 0.5539 | 0.4623 | 0.7101 | 0.4360 | 0.5107 | 0.7163 | 0.7887 | 0.0306 | 0.0528 |
| 0.1067 | 0.3668 | 0.1043 | 0.4999 | 0.7017 | 0.6196 | 0.6276 | 0.7630 | 0.3078 | 0.2451 |
| 0.1384 | 0.4044 | 0.1384 | 0.5119 | 0.1384 | 0.3151 | 0.1384 | 0.6069 | 0.0000 | 0.0001 |
| 0.4675 | 0.6034 | 0.4462 | 0.7043 | 0.7669 | 0.6352 | 0.8898 | 0.8246 | 0.0000 | 0.0000 |
| 0.8035 | 0.7040 | 0.4022 | 0.6946 | 0.3993 | 0.4941 | 0.1041 | 0.5714 | 0.0494 | 0.0742 |
| 0.1360 | 0.4035 | 0.0331 | 0.3519 | 0.1216 | 0.3058 | 0.2889 | 0.6600 | 0.0769 | 0.0976 |
| 0.1608 | 0.4185 | 0.1873 | 0.5888 | 0.2845 | 0.4337 | 0.3466 | 0.6648 | 0.0410 | 0.0641 |
| 0.2741 | 0.5014 | 0.1301 | 0.5071 | 0.7335 | 0.6280 | 0.8045 | 0.8058 | 0.0303 | 0.0524 |
| 0.7282 | 0.6789 | 0.9955 | 0.8533 | 0.5619 | 0.5716 | 0.0109 | 0.3368 | 0.0002 | 0.0021 |
| 0.0150 | 0.2237 | 0.7701 | 0.8147 | 0.6093 | 0.5883 | 0.6370 | 0.7663 | 0.0021 | 0.0096 |
| 0.6199 | 0.6579 | 0.2555 | 0.6326 | 0.0598 | 0.2308 | 0.1159 | 0.5885 | 0.0379 | 0.0612 |
| 0.0731 | 0.3355 | 0.2395 | 0.6241 | 0.4581 | 0.5239 | 0.2827 | 0.6595 | 0.2040 | 0.1915 |
| 0.5099 | 0.6224 | 0.0476 | 0.3955 | 0.0200 | 0.1425 | 0.2455 | 0.6504 | 0.0000 | 0.0000 |
| 0.0983 | 0.3626 | 0.2881 | 0.6500 | 0.7070 | 0.6201 | 0.9133 | 0.8246 | 0.5331 | 0.3444 |
| 0.6778 | 0.6642 | 0.7900 | 0.8177 | 0.3670 | 0.4812 | 0.0085 | 0.3368 | 0.1845 | 0.1811 |
| 0.3125 | 0.5330 | 0.3469 | 0.6614 | 0.3766 | 0.4819 | 0.1081 | 0.5768 | 0.0218 | 0.0433 |
| 0.6767 | 0.6642 | 0.5073 | 0.7340 | 0.1842 | 0.3581 | 0.1708 | 0.6234 | 0.9476 | 0.4817 |
| 0.1049 | 0.3668 | 0.9631 | 0.8486 | 0.6934 | 0.6181 | 0.1695 | 0.6229 | 0.1507 | 0.1586 |
| 0.3573 | 0.5543 | 0.7261 | 0.8004 | 0.7583 | 0.6335 | 0.4682 | 0.6992 | 0.0492 | 0.0742 |
| 0.9251 | 0.7286 | 0.9878 | 0.8533 | 0.1913 | 0.3598 | 0.8388 | 0.8170 | 0.0100 | 0.0251 |
| 0.0945 | 0.3584 | 0.3305 | 0.6550 | 0.3149 | 0.4505 | 0.9649 | 0.8347 | 0.0731 | 0.0943 |
| 0.9468 | 0.7353 | 0.4583 | 0.7101 | 0.6000 | 0.5865 | 0.0185 | 0.3996 | 0.0221 | 0.0435 |
| 0.5697 | 0.6398 | 0.8019 | 0.8177 | 0.4610 | 0.5240 | 0.9299 | 0.8246 | 0.0266 | 0.0489 |
| 0.3018 | 0.5249 | 0.7824 | 0.8177 | 0.7287 | 0.6254 | 0.4090 | 0.6770 | 0.4171 | 0.2998 |
| 0.2845 | 0.5094 | 0.5619 | 0.7608 | 0.2518 | 0.4091 | 0.1519 | 0.6131 | 0.3660 | 0.2750 |
| 0.6742 | 0.6642 | 0.6761 | 0.7856 | 0.6743 | 0.6152 | 0.2611 | 0.6561 | 0.0182 | 0.0381 |
| 0.0123 | 0.2237 | 0.0028 | 0.1565 | 0.0053 | 0.0694 | 0.7710 | 0.8025 | 0.9922 | 0.4951 |
| 0.5456 | 0.6273 | 0.3558 | 0.6631 | 0.0014 | 0.0375 | 0.2886 | 0.6600 | 0.6228 | 0.3782 |
| 0.1828 | 0.4361 | 0.1997 | 0.5977 | 0.0001 | 0.0242 | 0.0497 | 0.5116 | 0.0687 | 0.0891 |
| 0.1481 | 0.4179 | 0.2465 | 0.6309 | 0.0002 | 0.0255 | 0.0585 | 0.5136 | 0.1114 | 0.1272 |
| 0.2001 | 0.4548 | 0.4284 | 0.7007 | 0.0001 | 0.0203 | 0.1965 | 0.6258 | 0.2841 | 0.2328 |
| 0.1543 | 0.4185 | 0.4516 | 0.7097 | 0.0002 | 0.0255 | 0.1770 | 0.6258 | 0.2505 | 0.2180 |
| 0.7958 | 0.7021 | 0.4858 | 0.7227 | 0.0324 | 0.1796 | 0.7923 | 0.8058 | 0.8897 | 0.4660 |
| 0.5755 | 0.6410 | 0.5837 | 0.7622 | 0.1506 | 0.3199 | 0.7009 | 0.7879 | 0.4347 | 0.3093 |
| 0.4078 | 0.5723 | 0.8446 | 0.8286 | 0.9955 | 0.6909 | 0.1862 | 0.6258 | 0.7959 | 0.4426 |
| 0.4163 | 0.5794 | 0.9669 | 0.8487 | 0.4738 | 0.5264 | 0.8855 | 0.8246 | 0.7589 | 0.4315 |
| 0.6888 | 0.6642 | 0.4310 | 0.7007 | 0.6729 | 0.6152 | 0.5531 | 0.7345 | 0.4598 | 0.3224 |
| 0.9560 | 0.7367 | 0.1485 | 0.5300 | 0.7182 | 0.6238 | 0.6817 | 0.7844 | 0.0009 | 0.0054 |
| 0.8659 | 0.7219 | 0.0909 | 0.4917 | 0.8023 | 0.6445 | 0.3048 | 0.6607 | 0.0073 | 0.0194 |
| 0.3777 | 0.5570 | 0.6525 | 0.7782 | 0.4441 | 0.5182 | 0.7681 | 0.8025 | 0.1394 | 0.1499 |
| 0.8549 | 0.7193 | 0.0939 | 0.4999 | 0.1014 | 0.2848 | 0.5744 | 0.7391 | 0.0010 | 0.0057 |
| 0.8442 | 0.7175 | 0.2739 | 0.6478 | 0.0382 | 0.1918 | 0.0207 | 0.3996 | 0.8867 | 0.4658 |
| 0.4479 | 0.5997 | 0.9573 | 0.8469 | 0.3567 | 0.4796 | 0.4618 | 0.6929 | 0.0376 | 0.0612 |
| 0.6446 | 0.6641 | 0.6031 | 0.7706 | 0.8887 | 0.6670 | 0.6823 | 0.7844 | 0.4552 | 0.3209 |
| 0.8723 | 0.7219 | 0.6181 | 0.7771 | 0.1380 | 0.3151 | 0.0177 | 0.3996 | 0.5846 | 0.3632 |
| 0.1909 | 0.4413 | 0.2954 | 0.6533 | 0.2476 | 0.4052 | 0.2333 | 0.6380 | 0.0271 | 0.0494 |
| 0.5430 | 0.6273 | 0.1404 | 0.5144 | 0.3389 | 0.4742 | 0.0591 | 0.5136 | 0.0018 | 0.0087 |
| 0.4807 | 0.6122 | 0.9213 | 0.8444 | 0.8152 | 0.6465 | 0.3742 | 0.6648 | 0.2981 | 0.2403 |
| 0.4962 | 0.6198 | 0.9230 | 0.8444 | 0.1550 | 0.3248 | 0.4237 | 0.6821 | 0.0052 | 0.0160 |
| 0.0552 | 0.3182 | 0.0011 | 0.1535 | 0.0149 | 0.1169 | 0.3135 | 0.6635 | 0.8535 | 0.4600 |
| 0.8077 | 0.7043 | 0.0658 | 0.4504 | 0.7758 | 0.6352 | 0.0584 | 0.5136 | 0.2472 | 0.2169 |
| 0.0621 | 0.3199 | 0.4817 | 0.7227 | 0.0885 | 0.2629 | 0.9214 | 0.8246 | 0.0010 | 0.0057 |
| 0.8620 | 0.7201 | 0.4764 | 0.7213 | 0.1381 | 0.3151 | 0.1588 | 0.6131 | 0.7875 | 0.4405 |
| 0.2031 | 0.4570 | 0.0009 | 0.1535 | 0.1054 | 0.2908 | 0.0038 | 0.2605 | 0.0605 | 0.0831 |
| 0.4960 | 0.6198 | 0.7272 | 0.8004 | 0.9934 | 0.6909 | 0.4467 | 0.6869 | 0.2696 | 0.2257 |
| 0.0148 | 0.2237 | 0.0215 | 0.2928 | 0.0727 | 0.2474 | 0.3632 | 0.6648 | 0.0506 | 0.0749 |
| 0.7402 | 0.6835 | 0.8095 | 0.8177 | 0.0357 | 0.1918 | 0.8775 | 0.8246 | 0.1285 | 0.1411 |
| 0.9936 | 0.7431 | 0.8548 | 0.8302 | 0.8959 | 0.6700 | 0.5191 | 0.7111 | 0.7091 | 0.4130 |
| 0.4315 | 0.5919 | 0.2647 | 0.6395 | 0.5068 | 0.5459 | 0.2498 | 0.6549 | 0.0083 | 0.0216 |
| 0.6429 | 0.6641 | 0.8989 | 0.8441 | 0.2135 | 0.3747 | 0.7462 | 0.7941 | 0.6143 | 0.3757 |
| 0.9181 | 0.7281 | 0.5819 | 0.7622 | 0.7375 | 0.6296 | 0.3699 | 0.6648 | 0.0183 | 0.0381 |
| 0.3293 | 0.5435 | 0.5596 | 0.7591 | 0.9101 | 0.6736 | 0.8422 | 0.8170 | 0.0839 | 0.1038 |
| 0.1072 | 0.3668 | 0.8134 | 0.8177 | 0.1042 | 0.2888 | 0.5762 | 0.7401 | 0.5811 | 0.3621 |
| 0.0089 | 0.2237 | 0.0933 | 0.4999 | 0.3793 | 0.4819 | 0.1513 | 0.6131 | 0.1199 | 0.1344 |
| 0.7469 | 0.6849 | 0.5852 | 0.7622 | 0.4519 | 0.5216 | 0.0705 | 0.5225 | 0.0194 | 0.0395 |
| 0.1489 | 0.4179 | 0.5909 | 0.7629 | 0.2975 | 0.4407 | 0.2577 | 0.6561 | 0.0467 | 0.0713 |
| 0.1383 | 0.4044 | 0.3214 | 0.6550 | 0.5284 | 0.5530 | 0.2772 | 0.6595 | 0.0363 | 0.0606 |
| 0.5431 | 0.6273 | 0.4554 | 0.7101 | 0.0720 | 0.2465 | 0.3750 | 0.6648 | 0.5111 | 0.3375 |
| 0.3658 | 0.5570 | 0.0411 | 0.3763 | 0.3497 | 0.4770 | 0.7832 | 0.8048 | 0.2237 | 0.2050 |
| 0.3677 | 0.5570 | 0.0004 | 0.1535 | 0.1418 | 0.3151 | 0.0041 | 0.2605 | 0.4785 | 0.3275 |
| 0.0411 | 0.3026 | 0.2691 | 0.6441 | 0.0766 | 0.2530 | 0.4934 | 0.7056 | 0.0042 | 0.0141 |
| 0.0250 | 0.2518 | 0.0767 | 0.4788 | 0.4923 | 0.5374 | 0.2424 | 0.6488 | 0.4526 | 0.3196 |
| 0.6633 | 0.6642 | 0.9520 | 0.8469 | 0.7176 | 0.6238 | 0.0677 | 0.5225 | 0.3230 | 0.2519 |
| 0.5513 | 0.6285 | 0.0441 | 0.3767 | 0.7912 | 0.6398 | 0.3685 | 0.6648 | 0.5167 | 0.3386 |
| 0.3600 | 0.5547 | 0.0090 | 0.2084 | 0.2549 | 0.4095 | 0.4152 | 0.6821 | 0.6678 | 0.4002 |
| 0.8153 | 0.7074 | 0.9648 | 0.8486 | 0.9283 | 0.6736 | 0.2162 | 0.6280 | 0.2557 | 0.2192 |
| 0.4076 | 0.5723 | 0.0512 | 0.4026 | 0.0907 | 0.2632 | 0.7910 | 0.8058 | 0.5118 | 0.3375 |
| 0.6762 | 0.6642 | 0.2198 | 0.6071 | 0.2159 | 0.3760 | 0.0655 | 0.5225 | 0.5728 | 0.3593 |
| 0.6008 | 0.6551 | 0.4372 | 0.7007 | 0.7578 | 0.6335 | 0.0038 | 0.2605 | 0.5107 | 0.3375 |
| 0.2163 | 0.4709 | 0.0197 | 0.2928 | 0.6585 | 0.6121 | 0.0220 | 0.3996 | 0.4058 | 0.2962 |
| 0.3261 | 0.5421 | 0.0531 | 0.4065 | 0.1913 | 0.3598 | 0.1867 | 0.6258 | 0.5520 | 0.3536 |
| 0.1769 | 0.4322 | 0.0720 | 0.4676 | 0.1135 | 0.2962 | 0.2244 | 0.6317 | 0.0711 | 0.0920 |
| 0.8866 | 0.7219 | 0.9803 | 0.8530 | 0.3052 | 0.4468 | 0.1209 | 0.5944 | 0.8892 | 0.4660 |
| 0.2347 | 0.4709 | 0.6476 | 0.7782 | 0.1966 | 0.3624 | 0.9368 | 0.8269 | 0.1026 | 0.1198 |
| 0.9182 | 0.7281 | 0.2786 | 0.6478 | 0.4006 | 0.4941 | 0.0192 | 0.3996 | 0.2337 | 0.2112 |
| 0.2169 | 0.4709 | 0.0645 | 0.4455 | 0.3039 | 0.4468 | 0.1130 | 0.5856 | 0.0395 | 0.0625 |
| 0.7309 | 0.6795 | 0.8522 | 0.8302 | 0.9366 | 0.6736 | 0.9869 | 0.8349 | 0.0162 | 0.0350 |
| 0.8551 | 0.7193 | 0.5852 | 0.7622 | 0.9739 | 0.6846 | 0.7663 | 0.8020 | 0.2826 | 0.2328 |
| 0.5199 | 0.6257 | 0.1392 | 0.5126 | 0.8431 | 0.6536 | 0.0115 | 0.3368 | 0.4658 | 0.3239 |
| 0.0257 | 0.2518 | 0.1662 | 0.5588 | 0.8157 | 0.6465 | 0.4336 | 0.6821 | 0.3360 | 0.2593 |
| 0.3816 | 0.5570 | 0.8485 | 0.8286 | 0.1153 | 0.2984 | 0.4237 | 0.6821 | 0.0043 | 0.0142 |
| 0.5961 | 0.6550 | 0.0864 | 0.4917 | 0.3655 | 0.4812 | 0.8274 | 0.8167 | 0.0229 | 0.0442 |
| 0.0133 | 0.2237 | 0.5016 | 0.7316 | 0.4361 | 0.5107 | 0.4354 | 0.6835 | 0.0009 | 0.0054 |
| 0.4105 | 0.5748 | 0.5814 | 0.7622 | 0.2873 | 0.4338 | 0.2904 | 0.6600 | 0.0171 | 0.0365 |
| 0.7113 | 0.6724 | 0.3645 | 0.6705 | 0.9505 | 0.6745 | 0.2684 | 0.6561 | 0.1405 | 0.1506 |
| 0.8270 | 0.7149 | 0.8739 | 0.8353 | 0.7997 | 0.6441 | 0.3438 | 0.6648 | 0.9177 | 0.4744 |
| 0.0157 | 0.2237 | 0.1424 | 0.5169 | 0.3744 | 0.4819 | 0.0016 | 0.2465 | 0.0877 | 0.1054 |
| 0.6815 | 0.6642 | 0.1980 | 0.5977 | 0.1291 | 0.3124 | 0.1424 | 0.6069 | 0.7092 | 0.4130 |
| 0.5987 | 0.6550 | 0.8152 | 0.8179 | 0.0579 | 0.2296 | 0.8199 | 0.8124 | 0.0279 | 0.0500 |
| 0.0920 | 0.3547 | 0.6649 | 0.7800 | 0.0032 | 0.0549 | 0.4317 | 0.6821 | 0.0099 | 0.0250 |
| 0.6625 | 0.6642 | 0.1760 | 0.5741 | 0.0355 | 0.1918 | 0.4716 | 0.7014 | 0.8212 | 0.4505 |
| 0.1134 | 0.3697 | 0.0027 | 0.1565 | 0.0507 | 0.2160 | 0.0558 | 0.5136 | 0.2204 | 0.2023 |
| 0.3752 | 0.5570 | 0.2783 | 0.6478 | 0.3219 | 0.4585 | 0.0940 | 0.5648 | 0.0103 | 0.0257 |
| 0.1744 | 0.4322 | 0.7529 | 0.8130 | 0.6642 | 0.6129 | 0.9089 | 0.8246 | 0.5743 | 0.3594 |
| 0.1881 | 0.4380 | 0.5775 | 0.7622 | 0.2116 | 0.3734 | 0.0316 | 0.4825 | 0.0368 | 0.0612 |
| 0.7950 | 0.7021 | 0.9412 | 0.8461 | 0.3452 | 0.4742 | 0.2864 | 0.6600 | 0.0636 | 0.0854 |
| 0.8308 | 0.7149 | 0.8488 | 0.8286 | 0.1952 | 0.3624 | 0.1080 | 0.5768 | 0.0860 | 0.1047 |
| 0.0520 | 0.3182 | 0.1014 | 0.4999 | 0.5541 | 0.5673 | 0.4512 | 0.6869 | 0.0250 | 0.0466 |
| 0.0705 | 0.3281 | 0.3233 | 0.6550 | 0.2834 | 0.4331 | 0.7113 | 0.7885 | 0.6766 | 0.4034 |
| 0.0898 | 0.3523 | 0.3720 | 0.6705 | 0.3558 | 0.4796 | 0.3782 | 0.6649 | 0.4092 | 0.2963 |
| 0.9124 | 0.7281 | 0.2877 | 0.6500 | 0.6969 | 0.6188 | 0.9015 | 0.8246 | 0.4685 | 0.3248 |
| 0.9691 | 0.7383 | 0.3318 | 0.6550 | 0.1979 | 0.3636 | 0.6888 | 0.7844 | 0.0000 | 0.0004 |
| 0.0896 | 0.3523 | 0.0896 | 0.4917 | 0.8171 | 0.6465 | 0.1134 | 0.5856 | 0.0008 | 0.0052 |
| 0.2695 | 0.4963 | 0.1990 | 0.5977 | 0.0140 | 0.1128 | 0.1891 | 0.6258 | 0.2818 | 0.2328 |
| 0.2589 | 0.4855 | 0.3302 | 0.6550 | 0.1413 | 0.3151 | 0.8427 | 0.8170 | 0.1678 | 0.1710 |
| 0.7124 | 0.6724 | 0.6503 | 0.7782 | 0.0063 | 0.0739 | 0.0688 | 0.5225 | 0.0155 | 0.0342 |
| 0.4993 | 0.6198 | 0.9976 | 0.8533 | 0.0529 | 0.2209 | 0.5532 | 0.7345 | 0.0407 | 0.0640 |
| 0.9086 | 0.7281 | 0.7298 | 0.8004 | 0.0019 | 0.0485 | 0.2227 | 0.6293 | 0.3524 | 0.2679 |
| 0.2539 | 0.4788 | 0.5596 | 0.7591 | 0.1768 | 0.3536 | 0.7302 | 0.7936 | 0.7982 | 0.4432 |
| 0.2341 | 0.4709 | 0.3709 | 0.6705 | 0.1343 | 0.3151 | 0.3936 | 0.6735 | 0.7564 | 0.4315 |
| 0.6742 | 0.6642 | 0.7148 | 0.8004 | 0.3427 | 0.4742 | 0.9759 | 0.8347 | 0.5125 | 0.3375 |
| 0.8336 | 0.7158 | 0.9975 | 0.8533 | 0.7939 | 0.6402 | 0.7510 | 0.7970 | 0.0011 | 0.0059 |
| 0.3926 | 0.5655 | 0.7650 | 0.8146 | 0.6094 | 0.5883 | 0.4466 | 0.6869 | 0.5022 | 0.3355 |
| 0.3359 | 0.5484 | 0.4010 | 0.6946 | 0.3364 | 0.4727 | 0.9498 | 0.8292 | 0.4913 | 0.3300 |
| 0.3505 | 0.5527 | 0.3075 | 0.6550 | 0.3148 | 0.4505 | 0.0217 | 0.3996 | 0.5377 | 0.3462 |
| 0.1123 | 0.3697 | 0.2989 | 0.6533 | 0.2615 | 0.4138 | 0.4434 | 0.6869 | 0.0378 | 0.0612 |
| 0.6565 | 0.6642 | 0.1065 | 0.4999 | 0.0215 | 0.1450 | 0.2830 | 0.6595 | 0.1992 | 0.1891 |
| 0.5306 | 0.6262 | 0.8189 | 0.8191 | 0.0617 | 0.2329 | 0.6377 | 0.7663 | 0.6162 | 0.3762 |
| 0.9648 | 0.7367 | 0.4243 | 0.7007 | 0.1305 | 0.3131 | 0.4820 | 0.7017 | 0.0026 | 0.0110 |
| 0.2355 | 0.4709 | 0.1048 | 0.4999 | 0.5344 | 0.5566 | 0.3235 | 0.6635 | 0.6657 | 0.3994 |
| 0.8213 | 0.7117 | 0.2647 | 0.6395 | 0.5869 | 0.5809 | 0.0014 | 0.2465 | 0.3560 | 0.2698 |
| 0.5061 | 0.6222 | 0.4219 | 0.7007 | 0.5221 | 0.5517 | 0.5058 | 0.7061 | 0.2495 | 0.2179 |
| 0.1336 | 0.4035 | 0.0028 | 0.1565 | 0.6825 | 0.6153 | 0.0094 | 0.3368 | 0.4090 | 0.2963 |
| 0.2403 | 0.4709 | 0.7771 | 0.8174 | 0.0178 | 0.1327 | 0.7288 | 0.7936 | 0.2284 | 0.2085 |
| 0.2795 | 0.5062 | 0.6368 | 0.7782 | 0.9367 | 0.6736 | 0.4955 | 0.7056 | 0.0804 | 0.1011 |
| 0.5879 | 0.6494 | 0.1693 | 0.5645 | 0.6060 | 0.5877 | 0.5703 | 0.7388 | 0.0000 | 0.0001 |
| 0.0115 | 0.2237 | 0.4360 | 0.7007 | 0.0861 | 0.2629 | 0.3434 | 0.6648 | 0.0035 | 0.0133 |
| 0.0867 | 0.3518 | 0.2487 | 0.6323 | 0.1627 | 0.3342 | 0.6038 | 0.7510 | 0.0606 | 0.0831 |
| 0.3818 | 0.5570 | 0.7089 | 0.8004 | 0.0140 | 0.1128 | 0.8457 | 0.8170 | 0.2577 | 0.2193 |
| 0.1750 | 0.4322 | 0.3500 | 0.6627 | 0.0104 | 0.0958 | 0.4330 | 0.6821 | 0.8901 | 0.4660 |
| 0.9794 | 0.7411 | 0.6376 | 0.7782 | 0.2161 | 0.3760 | 0.6846 | 0.7844 | 0.9868 | 0.4950 |
| 0.2391 | 0.4709 | 0.9468 | 0.8469 | 0.8487 | 0.6541 | 0.1787 | 0.6258 | 0.0127 | 0.0295 |
| 0.6179 | 0.6579 | 0.2216 | 0.6076 | 0.1772 | 0.3536 | 0.5315 | 0.7215 | 0.4621 | 0.3233 |
| 0.0096 | 0.2237 | 0.7459 | 0.8088 | 0.8072 | 0.6465 | 0.4859 | 0.7022 | 0.5148 | 0.3383 |
| 0.2347 | 0.4709 | 0.0221 | 0.2928 | 0.0265 | 0.1631 | 0.8308 | 0.8170 | 0.5969 | 0.3679 |
| 0.2772 | 0.5043 | 0.5690 | 0.7622 | 0.9746 | 0.6846 | 0.0890 | 0.5648 | 0.6857 | 0.4056 |
| 0.0546 | 0.3182 | 0.0222 | 0.2928 | 0.0052 | 0.0694 | 0.1658 | 0.6172 | 0.8634 | 0.4604 |
| 0.6380 | 0.6641 | 0.4782 | 0.7221 | 0.0501 | 0.2160 | 0.6364 | 0.7663 | 0.0127 | 0.0295 |
| 0.9639 | 0.7367 | 0.8949 | 0.8427 | 0.7244 | 0.6254 | 0.2440 | 0.6504 | 0.0064 | 0.0179 |
| 0.0016 | 0.1952 | 0.0001 | 0.0630 | 0.0003 | 0.0255 | 0.3357 | 0.6648 | 0.0032 | 0.0126 |
| 0.0126 | 0.2237 | 0.1090 | 0.4999 | 0.0639 | 0.2359 | 0.4078 | 0.6765 | 0.0668 | 0.0886 |
| 0.3674 | 0.5570 | 0.7912 | 0.8177 | 0.8582 | 0.6565 | 0.7148 | 0.7887 | 0.1688 | 0.1712 |
| 0.9605 | 0.7367 | 0.1617 | 0.5502 | 0.2395 | 0.3982 | 0.7798 | 0.8048 | 0.6769 | 0.4034 |
| 0.7466 | 0.6849 | 0.0013 | 0.1535 | 0.0715 | 0.2465 | 0.4734 | 0.7017 | 0.1963 | 0.1881 |
| 0.7927 | 0.7012 | 0.2991 | 0.6533 | 0.2568 | 0.4104 | 0.5901 | 0.7484 | 0.5397 | 0.3467 |
| 0.0046 | 0.2237 | 0.0278 | 0.3156 | 0.1120 | 0.2946 | 0.7239 | 0.7918 | 0.3254 | 0.2534 |

TABLE 10

| | Lipid species | | | | | | | | |
|---|---|---|---|---|---|---|---|---|---|
| | Bt:WT-M1 | Bt:WT-M2 | Bt:WT-F3 | Bt:WT-M18 | Bt:WT-F19 | Bt:BSHIVMOL361-M4 | Bt:BSHIVMOL361-M5 | Bt:BSHIVMOL361-M6 | Bt:BSHIVMOL361-F7 |
| CE 12:0 | 2.8205287 | 2.6197959 | 2.6684662 | 1.5551072 | 0.7086725 | 3.7873036 | 2.3219351 | 1.5967224 | 3.7409552 |
| CE 14:0 | 6.4658433 | 6.141865 | 4.746562 | 5.1077041 | 3.1688251 | 7.0848343 | 5.9334441 | 4.6227474 | 6.7166259 |
| CE 14:1 | 4.6458692 | 3.9346624 | 3.9778745 | 2.9961049 | 1.1777462 | 5.5002478 | 3.4343202 | 2.7790632 | 5.9470227 |
| CE 15:0 | 7.7324218 | 8.0865727 | 5.9072193 | 6.2898184 | 4.351373 | 8.9020616 | 7.8689673 | 5.5831207 | 8.2884003 |
| CE 16:0 | 94.824119 | 110.6156 | 66.643962 | 92.883835 | 102.39789 | 102.64703 | 117.33327 | 91.306014 | 104.91616 |
| CE 16:1 | 39.880856 | 36.48874 | 37.473816 | 34.90033 | 40.866078 | 48.55576 | 36.965479 | 35.164076 | 59.936962 |
| CE 17:0 | 5.4668457 | 6.77396 | 4.7752674 | 4.1449322 | 5.7486699 | 6.0347891 | 5.1248054 | 4.8563874 | 8.8076864 |
| CE 18:0 | 6.3159394 | 10.467799 | 6.5826592 | 5.9566261 | 10.532525 | 7.9967056 | 7.6187395 | 8.9330937 | 9.8273594 |
| CE 18:1 | 111.49877 | 117.22932 | 93.758816 | 106.76781 | 161.71018 | 133.15325 | 124.77739 | 113.8683 | 174.19722 |
| CE 18:2 | 1269.2477 | 1336.769 | 630.02152 | 1305.4159 | 845.00891 | 1211.9108 | 1551.6136 | 1238.8906 | 1009.6038 |
| CE 18:3 | 32.711933 | 27.695203 | 22.682603 | 24.039716 | 14.99627 | 33.995486 | 28.873226 | 22.52118 | 35.130521 |
| CE 18:4 | 2.8879007 | 2.5508695 | 2.452469 | 1.6979054 | 0.7861928 | 3.3322451 | 2.0003131 | 1.6622857 | 3.5794448 |
| CE 20:0 | 182.01825 | 206.57895 | 141.48872 | 142.84837 | 74.568354 | 280.55408 | 246.67964 | 170.17931 | 214.65787 |
| CE 20:1 | 64.637982 | 45.724015 | 48.898568 | 24.266267 | 7.8449479 | 99.456283 | 35.5104 | 22.627029 | 76.698798 |
| CE 20:2 | 3.0316554 | 2.7334719 | 2.4809102 | 1.6285987 | 1.1665434 | 4.4698197 | 2.5348995 | 1.6221844 | 3.5719475 |
| CE 20:3 | 30.37984 | 29.895242 | 18.384904 | 26.915497 | 21.643408 | 33.240605 | 36.125895 | 26.328802 | 28.329695 |
| CE 20:4 | 651.39374 | 675.67728 | 451.34758 | 639.29292 | 566.06875 | 621.61545 | 727.91604 | 609.21378 | 643.76897 |
| CE 20:5 | 138.16244 | 138.97544 | 89.619751 | 136.96998 | 65.986675 | 137.7666 | 131.93953 | 128.62414 | 187.31013 |
| CE 22:0 | 4.0863535 | 4.5512026 | 3.2621362 | 3.650516 | 1.8772393 | 6.3029716 | 5.1685927 | 3.7459906 | 5.2218917 |
| CE 22:1 | 3.4158462 | 4.1205707 | 2.7336285 | 2.5159311 | 1.7572223 | 5.8519202 | 4.1874624 | 3.4442345 | 4.8870524 |
| CE 22:2 | 33.929538 | 36.256921 | 27.897029 | 25.602958 | 18.493493 | 44.610371 | 40.249936 | 28.660134 | 43.033129 |
| CE 22:4 | 4.0442349 | 4.5442532 | 4.5200824 | 3.0909189 | 1.6703763 | 6.1336069 | 3.9351662 | 2.9722126 | 6.7771842 |
| CE 22:5 | 7.9402796 | 7.9242285 | 5.4879236 | 10.043453 | 3.6377331 | 10.895008 | 8.142855 | 6.667991 | 9.0509172 |
| CE 22:6 | 191.60182 | 202.29939 | 109.88072 | 212.12826 | 119.57823 | 214.21908 | 249.06159 | 221.67861 | 200.00841 |
| CE 24:0 | 65.758526 | 88.389117 | 58.183396 | 61.384361 | 52.491947 | 87.666522 | 101.2581 | 72.096024 | 88.924235 |
| CE 24:1 | 15.800786 | 16.026549 | 12.423203 | 12.508993 | 5.8238023 | 19.861932 | 16.142133 | 13.267948 | 23.308043 |
| Cer d18:0/24:1 | 0.2097789 | | | | | | | | |
| Cer d18:1/14:0 | | | | | | | | | |
| Cer d18:1/16:0 | 0.2015476 | 0.1917517 | 0.1776299 | 0.1197856 | 0.1842449 | | 0.1398755 | 0.1102742 | 0.1898528 |
| Cer d18:1/18:0 | | | | | | | | | |
| Cer d18:1/20:0 | | | | | | | | | |
| Cer d18:1/22:0 | 0.6920071 | 0.7539929 | 0.358754 | 0.7713594 | 0.2184741 | 0.4934352 | 0.9425068 | 0.7410147 | 0.3199908 |
| Cer d18:1/24:0 | 1.1543339 | 1.2065794 | 0.850807 | 1.1765779 | 0.5977065 | 0.9571946 | 1.194078 | 1.0892943 | 0.8309422 |
| Cer d18:1/24:1 | 0.4964801 | 0.4353349 | 0.7819596 | 0.4677449 | 0.6711591 | 0.4986169 | 0.5404877 | 0.4433059 | 0.9395859 |
| DG 12:0_18:1 | | | | | | | | | 0.3027643 |
| DG 12:0_18:2 | | 0.6651082 | | | | | | | |
| DG 14:1_18:1 | 0.456045 | | | | | | | | |
| DG 15:0_18:2 | | 0.7340456 | | | | | | | |
| DG 16:0_16:0 | 0.9471072 | 0.9409531 | 0.4918631 | 0.4860837 | 0.4500826 | 0.4012716 | 0.2158316 | 0.2490216 | 0.6046182 |
| DG 16:0_16:1 | | 0.2748631 | | | | | | | |
| DG 16:0_18:0 | 0.9738081 | | | | | | | 0.9041611 | |
| DG 16:0_18:1 | 1.5573628 | 2.0883877 | 1.5003807 | 1.5245318 | 2.3672274 | 1.4156923 | 0.662902 | 0.9337346 | 1.1646436 |
| DG 16:0_18:2 | 5.3085512 | 6.3134349 | 4.353147 | 5.5242757 | 4.3865361 | 4.1342065 | 3.1455864 | 1.9398901 | |
| DG 16:0_22:6 | | | | 0.4334409 | 0.2647662 | | 0.318549 | 0.3027901 | |
| DG 16:1_18:1 | | | | | 0.5843421 | | | | |
| DG 16:1_18:2 | 1.082268 | 1.0611898 | 0.9882861 | 1.0769515 | 1.1685925 | | | | |
| DG 18:0_18:1 | | 0.2192722 | | | | | | | |
| DG 18:0_18:2 | 0.7351349 | 0.9163166 | 0.9143003 | 0.4729591 | 0.5063285 | 0.6612901 | | 0.4194545 | |
| DG 18:1_18:1 | 0.7258495 | 0.9077485 | 0.7681729 | 0.647318 | 2.2191516 | 0.530076 | 0.2434069 | 0.4676261 | 0.54863 |
| DG 18:1_18:2 | 10.92136 | 8.4705773 | 7.0087817 | 10.752033 | 11.018153 | 5.5386408 | 4.9573222 | 3.6461888 | |
| DG 18:1_20:1 | 0.4435185 | 0.3636486 | | | 0.2413693 | 0.2941025 | | | 0.3090783 |
| DG 18:1_20:2 | | | | | | | | | |
| DG 18:1_20:4 | | | | | 0.437331 | | | | |
| DG 18:1_22:6 | | | | 1.3012175 | 1.1680951 | | 0.9143947 | 0.7912277 | |
| DG 18:2_20:4 | | | | | 0.6011754 | | | | |
| DG 18:2_22:6 | | | | 4.5518966 | 1.6059874 | | 2.9375232 | 1.7543331 | |
| FA 12:0 | 3.8664393 | 2.425107 | 3.0372257 | 2.8102677 | 2.6469153 | 3.1337673 | 3.071337 | 2.8736802 | 3.1119497 |
| FA 14:0 | 22.789277 | 17.023753 | 20.371688 | 20.435894 | 17.083731 | 24.006095 | 23.82572 | 26.167148 | 23.098382 |
| FA 14:1 | 5.1422294 | 4.6537343 | 6.0306435 | 5.262259 | 4.5905857 | 5.8453821 | 5.6866108 | 5.7011175 | 5.9285047 |
| FA 15:0 | 11.112662 | 10.752546 | 11.57802 | 10.772517 | 9.5459917 | 10.684604 | 11.402378 | 11.029248 | 11.556327 |
| FA 16:0 | 190.51149 | 141.67621 | 190.6166 | 177.07567 | 153.52723 | 204.10549 | 210.7934 | 224.47445 | 206.90803 |
| FA 16:1 | 27.559952 | 18.759798 | 24.397578 | 23.221152 | 22.136746 | 28.126886 | 28.750823 | 31.412525 | 28.447076 |
| FA 17:0 | 11.797738 | 11.277556 | 12.632838 | 11.302934 | 10.220694 | 12.898102 | 12.757234 | 12.249012 | 12.817524 |
| FA 18:0 | 71.268227 | 51.802427 | 66.497146 | 58.287557 | 53.272444 | 67.020288 | 67.497201 | 65.564339 | 66.379066 |
| FA 18:1 | 68.773249 | 46.989814 | 67.078934 | 62.417572 | 52.212183 | 73.767482 | 83.11243 | 81.538768 | 80.801151 |
| FA 18:2 | 82.315519 | 46.598776 | 67.119004 | 79.867446 | 51.200358 | 73.657692 | 101.01527 | 90.735966 | 90.956178 |
| FA 18:3 | 8.3099989 | 5.0763046 | 6.5436243 | 9.617747 | 5.3961224 | 6.3801073 | 8.7722811 | 7.7268575 | 8.05871 |
| FA 18:4 | 1.4123905 | 1.2080058 | 1.3789627 | 1.4970608 | 0.937989 | 1.3001726 | 1.4456597 | 1.2679334 | 1.6099313 |
| FA 20:0 | 7.6508198 | 4.1567734 | 8.2689331 | 5.1085877 | 3.3380586 | 9.3832301 | 7.2763555 | 6.0817505 | 10.114626 |
| FA 20:1 | 6.677309 | 4.2147559 | 6.1757319 | 4.9204843 | 3.1416636 | 6.2157708 | 6.0971532 | 5.4506142 | 6.203513 |
| FA 20:2 | 1.9556585 | 1.5324989 | 2.0890886 | 1.8813294 | 1.4941689 | 2.2402655 | 2.2237762 | 1.912577 | 2.1586639 |
| FA 20:3 | 2.3588725 | 2.1457153 | 2.4215541 | 2.4011064 | 2.0361405 | 2.4758157 | 2.5757548 | 2.2433928 | 2.5411972 |
| FA 20:4 | 5.4373061 | 4.2648726 | 5.1055686 | 6.1167605 | 5.5972358 | 4.4885234 | 6.1759078 | 5.2897709 | 5.3080196 |
| FA 20:5 | 2.5039149 | 1.8907828 | 2.3896583 | 3.6140289 | 1.8608491 | 2.37195 | 3.1090194 | 2.6273052 | 2.6906945 |
| FA 22:0 | 3.3066335 | 3.0522546 | 3.7980334 | 3.1399232 | 3.1113403 | 3.5722035 | 3.7896794 | 3.8039068 | 4.1189881 |
| FA 22:1 | 3.4596428 | 3.2855745 | 4.9224876 | 3.8567565 | 3.0882855 | 4.4863584 | 4.5281177 | 5.121508 | 4.5193462 |
| FA 22:2 | 2.0614965 | 1.356067 | 2.0523042 | 1.52323 | 1.284399 | 1.7711761 | 1.6673192 | 1.6389668 | 1.8223979 |
| FA 22:4 | 1.4336785 | 1.4403398 | 1.5714955 | 1.3619841 | 1.2601264 | 1.5491645 | 1.5374407 | 1.2704374 | 1.6345007 |
| FA 22:5 | 7.4855619 | 3.7617243 | 6.0325457 | 6.0396357 | 4.7023034 | 4.9595861 | 5.4321507 | 3.99973 | 4.820024 |
| FA 22:6 | 7.4607314 | 5.6343261 | 6.4215594 | 10.25385 | 7.232963 | 8.0006422 | 9.8147395 | 7.991949 | 6.6864033 |
| FA 24:0 | 2.3304737 | 2.4191614 | 2.8331303 | 2.1684547 | 1.9525979 | 2.4602981 | 2.242451 | 2.2197227 | 2.5833424 |
| FA 24:1 | 2.9466557 | 3.4732579 | 3.9699698 | 2.9700243 | 2.4850425 | 3.5519963 | 3.281878 | 3.4335373 | 3.4138354 |
| HexCER d18:1/16:0 | 0.1545452 | 0.1698333 | 0.2656102 | 0.1507009 | 0.2100413 | | 0.2174336 | 0.2246891 | 0.3402799 |
| HexCER d18:1/18:0 | | | 0.1072293 | | | | | | |
| HexCER d18:1/20:0 | | | | | | | | | 0.1222956 |
| HexCER d18:1/22:0 | 0.4882548 | 0.4343526 | 0.3409506 | 0.6499328 | 0.3068234 | 0.3811911 | 0.5850652 | 0.474579 | 0.3983126 |
| HexCER d18:1/22:1 | | | | 0.1251582 | | 0.117292 | 0.1278583 | | |
| HexCER d18:1/24:0 | 0.2087666 | 0.1823239 | 0.238017 | 0.2369401 | 0.1983629 | 0.2221398 | 0.2038733 | 0.1976629 | 0.2386303 |
| HexCER d18:1/24:1 | 0.2168038 | 0.1301556 | 0.4398575 | 0.1990356 | 0.3423925 | 0.2887701 | 0.2537457 | 0.1719567 | 0.5675677 |
| LPC 15:0 | 0.7977903 | 0.7663533 | 0.978413 | 0.9026124 | 0.6267601 | 0.9883396 | 0.8026829 | 0.8219836 | 0.8653324 |
| LPC 16:0 | 155.20821 | 146.18904 | 210.58537 | 170.5265 | 154.20905 | 201.82997 | 219.51394 | 189.80873 | 191.94905 |
| LPC 16:1 | 2.0435477 | 1.6442511 | 2.9678857 | 2.4540187 | 2.4197006 | 3.0336872 | 2.5164388 | 2.1179704 | 3.4295186 |
| LPC 17:0 | 4.787934 | 4.8567631 | 4.8473287 | 5.5811757 | 4.1634045 | 4.5587688 | 5.2119209 | 4.7192896 | 5.2006645 |
| LPC 18:0 | 62.861994 | 64.297769 | 84.879009 | 83.665351 | 78.286489 | 60.123954 | 83.017364 | 74.249905 | 74.349691 |
| LPC 18:1 | 28.944221 | 23.131147 | 36.022812 | 32.813304 | 28.46606 | 46.962533 | 33.797436 | 27.357464 | 37.601073 |
| LPC 18:2 | 88.183061 | 65.210465 | 96.386285 | 102.63006 | 77.04481 | 108.1754 | 108.41232 | 87.290313 | 108.35115 |
| LPC 18:3 | 0.5743598 | | 0.5058941 | 0.7202616 | 0.495513 | | 0.5252252 | 0.4732598 | 0.6305416 |
| LPC 20:0 | 4.1813382 | 2.5060327 | 4.3893157 | 2.5157039 | 0.8246764 | 5.8938146 | 3.2044787 | 2.1014239 | 5.1302532 |
| LPC 20:1 | 1.408284 | 0.9094796 | 0.9315268 | 1.1037019 | 0.6786012 | 1.6092104 | 1.1354659 | 0.9569684 | 1.3161824 |
| LPC 20:2 | 0.8083987 | 0.6957641 | 0.6303142 | 0.7332158 | 0.659518 | 1.1835994 | 0.9032079 | 0.66905 | 0.8508304 |
| LPC 20:3 | 5.1385759 | 3.9881821 | 3.7485552 | 5.113636 | 5.5004993 | 5.8982847 | 5.2630298 | 3.9399311 | 4.2257873 |
| LPC 20:4 | 11.03309 | 8.4130512 | 9.7060455 | 12.856152 | 14.071344 | 9.0718493 | 12.048316 | 10.636414 | 11.881264 |
| LPC 20:5 | 0.5423802 | 0.5136143 | 0.7691267 | 1.2719164 | 0.534266 | 0.5104891 | 0.6059468 | 0.7952598 | 0.9635305 |
| LPC 22:5 | | | | 0.5832807 | | | | | |
| LPC 22:6 | 2.0886178 | 1.6363887 | 1.0574953 | 2.6557129 | 1.9706744 | 1.583261 | 2.2732971 | 2.5065484 | 1.7118868 |
| LPE 16:0 | 4.652431 | 4.9927175 | 6.6616515 | 4.7867155 | 4.1998727 | 4.9320033 | 5.1685804 | 4.8439743 | 5.4025164 |
| LPE 18:0 | 1.5216276 | 1.9123469 | 2.0088568 | 2.0816376 | 1.6796216 | 1.7326299 | 2.2097112 | 1.8207728 | 2.0085508 |
| LPE 18:1 | 0.8676029 | 0.9684716 | 1.1917729 | 0.9703606 | 0.9152206 | 1.071815 | 0.8754013 | 0.8200818 | 1.3734043 |
| LPE 18:2 | 2.2288526 | 1.5618217 | 1.8576233 | 2.2817746 | 1.1870492 | 1.7280446 | 2.5053641 | 1.8663292 | 2.5472963 |
| LPE 20:4 | 0.3769819 | | | | 0.4103775 | | | | |
| LacCER d18:1/16:0 | | | | | | | | | |
| LacCER d18:1/18:1 | | | | | | | | | |
| LacCER d18:1/22:0 | | | | 0.2105764 | 0.1884532 | | | | |
| PA 34:1 | 0.3623669 | 0.3614799 | 0.3400997 | 0.3103798 | | 0.41597 | 0.2350231 | 0.2030481 | 0.2546081 |
| PA 34:2 | 1.1125444 | 1.1475055 | 1.4342789 | 1.2677031 | 0.480573 | 1.4632951 | 1.0787222 | 1.0872302 | 1.1572885 |
| PA 34:3 | | | | 0 | | | | | |
| PA 34:4 | 2.1722584 | 2.2957797 | 2.304285 | 2.353546 | 2.3458993 | 2.2302908 | 2.1824796 | 2.2253009 | 2.3108795 |
| PA 34:5 | 0.5879593 | 0.5911502 | 0.530071 | 0.5119579 | 0.5851714 | 0.7286388 | 0.5623052 | 0.4593841 | 0.6883496 |
| PA 36:1 | 0.1399251 | 0.103333 | 0.180342 | 0.1570504 | | 0.2375611 | 0.1119514 | 0.1530752 | 0.137257 |
| PA 36:2 | 0.7181603 | 0.8546272 | 0.7649533 | 0.8647834 | 0.3404528 | 0.8092042 | 0.7135106 | 0.7199199 | 0.8171991 |
| PA 36:3 | 0.2750549 | 0.3330728 | 0.3369643 | | 0.1595041 | 0.3155845 | 0.1959801 | | 0.2897679 |
| PA 36:4 | 0.1699153 | | 0.1630779 | 0.2409078 | | 0.2268551 | 0.2015395 | | |
| PA 36:5 | 0.187425 | 0.3398523 | 0.3450296 | 0.2530776 | | 0.3858581 | 0.3409478 | 0.2951038 | 0.4955441 |
| PA 38:3 | | | | | | | | | |
| PA 38:4 | | 0.2413801 | 0.2797085 | 0.3604908 | | 0.2997618 | 0.3310787 | 0.2694869 | 0.3173505 |
| PA 38:6 | | | | | | | 0.1675676 | | 0.1860859 |
| PA 38:7 | | | | 0.2119445 | | | 0.2011904 | | |
| PA 40:6 | | | | | | | | | |
| PA 40:8 | | | | | | | | | 0.1710954 |
| PC 12:0_16:0 | 0.5312391 | | 0.3901075 | 0.4321636 | | 0.6136904 | | 0.3990097 | 0.5658538 |
| PC 12:0_18:0 | | | | | | | | | |
| PC 12:0_18:3 | 0.9516506 | 0.9515332 | 1.3564576 | 0.7136413 | | 1.4204447 | 0.7939776 | 0.6976693 | 1.1425444 |
| PC 14:0_18:1 | 0.9826846 | 0.8275452 | 0.6261948 | 1.036733 | 1.0026043 | 1.1598772 | 1.0163837 | 0.7421172 | 0.7698949 |
| PC 14:0_18:2 | 1.5941643 | 1.4554149 | 1.5548906 | 2.01402 | 1.1052436 | 1.8644396 | 1.8186553 | 1.5738887 | 1.5625056 |
| PC 14:0_18:3 | | | | | | | | | |
| PC 14:0_20:4 | | | | | | | | | |
| PC 14:1_20:0 | | | | 0.4102816 | | | | | |
| PC 15:0_18:1 | | 0.4706937 | | | | | 0.4712798 | | |
| PC 15:0_18:2 | 1.5695751 | 1.2999576 | 0.7124156 | 1.4165694 | 0.8481745 | 1.3999062 | 1.6911621 | 1.083718 | 1.045663 |
| PC 16:0_16:0 | 45.161806 | 42.14813 | 34.057428 | 41.286772 | 27.707005 | 38.755767 | 49.285155 | 43.10041 | 31.878298 |
| PC 16:0_16:1 | 3.1279784 | 2.9346373 | 3.9416696 | 2.9240387 | 2.4123967 | 3.5178683 | 3.0718615 | 2.5641246 | 2.8675079 |
| PC 16:0_18:0 | 52.313624 | 49.694056 | 36.031068 | 49.974547 | 36.189549 | 33.071971 | 43.333871 | 44.431305 | 39.172708 |
| PC 16:0_18:1 | 150.37293 | 122.29494 | 132.50178 | 136.43779 | 101.99567 | 186.85611 | 168.53326 | 135.02074 | 130.54623 |
| PC 16:0_18:2 | 717.07671 | 613.57614 | 570.53201 | 648.35576 | 409.96071 | 701.48124 | 854.80333 | 654.95543 | 571.26295 |
| PC 16:0_18:3 | 3.7210975 | 2.8220964 | 4.464971 | 4.3749783 | 2.1887837 | 3.2385628 | 4.5134929 | 2.9192946 | 3.8395465 |
| PC 16:0_20:1 | 4.2156887 | 2.6642052 | 3.0919691 | 2.9065428 | 1.237982 | 4.0133297 | 2.4271812 | 2.0966712 | 3.0936625 |
| PC 16:0_20:2 | 4.2605379 | 3.4048572 | 3.0943559 | 3.4630176 | 2.6453912 | 7.7781901 | 5.4577187 | 3.9151987 | 4.2172382 |
| PC 16:0_20:3 | 58.758251 | 55.061849 | 50.002818 | 49.255381 | 39.386775 | 75.411998 | 64.925099 | 53.338884 | 49.826803 |
| PC 16:0_20:4 | 195.12794 | 155.59508 | 202.39709 | 200.24218 | 139.087 | 193.42372 | 198.7081 | 172.6518 | 185.99668 |
| PC 16:0_20:5 | 20.512649 | 21.645695 | 27.765623 | 32.043352 | 13.751703 | 27.444764 | 22.670805 | 22.905607 | 39.464144 |
| PC 16:0_22:1 | 0.4661571 | | | | | 0.4811686 | | | |
| PC 16:0_22:2 | 0.5365972 | | | | | | | | |
| PC 16:0_22:4 | 2.1515552 | 1.9825612 | | | 1.3133325 | | | | |
| PC 16:0_22:5 | 18.856182 | 14.883168 | 13.36853 | 26.074011 | 6.1145466 | 26.096301 | 15.516014 | 14.769679 | 16.7086 |
| PC 16:0_22:6 | 261.55015 | 243.20883 | 199.09218 | 245.91159 | 114.30303 | 341.39018 | 293.78849 | 295.33079 | 280.01678 |
| PC 16:1_17:0 | | | | 0.847999 | | | | | |
| PC 16:1_18:0 | | 0.4287625 | 0.6602891 | | 0.3255653 | | 0.3742509 | | 0.5864761 |
| PC 16:1_18:1 | 4.7338655 | 3.2700366 | 4.0289137 | 4.5557039 | 4.2481329 | 5.163692 | 3.7844677 | 3.4015024 | 4.5517931 |
| PC 16:1_18:2 | 9.7178348 | 7.288176 | 8.6191036 | 12.306719 | 5.8060033 | 11.049385 | 11.117694 | 8.0805665 | 9.968546 |
| PC 16:1_20:0 | | | 0.3829046 | | | | | | 0.4077208 |
| PC 17:0_18:1 | 1.1949968 | 0.9936145 | 0.7784824 | 1.3111342 | 0.6758588 | 0.9719682 | 1.2057371 | 0.9717043 | 1.2166262 |
| PC 17:0_18:2 | 6.5548207 | 5.8891959 | 4.3938884 | 5.9458917 | 3.643436 | 5.3814728 | 6.5466363 | 5.3323982 | 4.6167551 |
| PC 17:0_20:3 | 1.1478307 | 1.2387562 | 1.8389309 | 1.2652836 | 1.0588994 | 1.8622108 | | 1.6838527 | 1.5903021 |
| PC 17:0_20:4 | 7.3487346 | 5.8982442 | 5.6766055 | 5.6844708 | 4.3195064 | 6.1059494 | 5.6725041 | 5.0926687 | 6.2811532 |
| PC 17:0_22:6 | | | | 7.7349388 | | 7.3941676 | 4.9454498 | | 6.2410566 |
| PC 18:0_18:0 | 1.0165327 | 0.934812 | 0.864578 | 0.945357 | 0.8968441 | 0.7552522 | 0.7453348 | 0.8010022 | 0.7201723 |
| PC 18:0_18:1 | 21.409108 | 19.282087 | 18.947198 | 23.375787 | 14.593385 | 21.284393 | 20.859985 | 18.264372 | 18.603444 |
| PC 18:0_18:2 | 316.10263 | 289.52771 | 249.29724 | 321.00108 | 210.26652 | 249.23208 | 325.60727 | 282.99265 | 259.35498 |
| PC 18:0_18:3 | 0.9656271 | 0.9971363 | 1.0104403 | 0.986848 | 0.7432077 | 0.8496963 | 0.9000294 | | 1.0960125 |
| PC 18:0_20:0 | 5.5726356 | 5.3516609 | 5.3626152 | 5.4256627 | 2.8863949 | 5.4951477 | 5.7611424 | 4.8107611 | 5.8982747 |
| PC 18:0_20:1 | 1.1205816 | 0.7241698 | 1.135101 | 0.7220003 | | 1.0527433 | 0.4859271 | 0.6747496 | 0.8406792 |
| PC 18:0_20:2 | 1.4943641 | 1.2228866 | 1.0019389 | 1.2344191 | 0.9365066 | 1.9716078 | 1.4342582 | 1.2016526 | 1.4273453 |
| PC 18:0_20:3 | 23.348929 | 22.279982 | 21.405096 | 23.98217 | 19.531175 | 23.335819 | 24.89359 | 23.793304 | 22.215929 |
| PC 18:0_20:4 | 99.44627 | 82.690822 | 125.14022 | 125.91229 | 87.568291 | 77.669151 | 100.70658 | 95.962686 | 114.3578 |
| PC 18:0_20:5 | 10.210375 | 12.921765 | 13.123048 | 17.257322 | 7.3455388 | 11.340938 | 10.114149 | 12.615384 | 22.421087 |
| PC 18:0_22:4 | | | | | | | | | |
| PC 18:0_22:5 | 3.7815329 | 4.1919711 | 4.9857778 | 8.8846153 | 2.4696612 | 5.6029272 | 4.3147222 | 3.5984808 | 5.0716102 |
| PC 18:0_22:6 | 64.546072 | 63.386773 | 55.41225 | 76.386949 | 40.656311 | 71.295631 | 72.643411 | 79.013509 | 63.536882 |
| PC 18:1_18:1 | 5.9198401 | 5.3350813 | 4.2002282 | 5.6473619 | 4.6586815 | 6.091454 | 5.0068441 | 3.8849068 | 5.5629563 |
| PC 18:1_18:2 | 70.823563 | 60.86387 | 46.617588 | 60.221491 | 47.9215 | 61.238711 | 62.581128 | 45.59193 | 57.787173 |
| PC 18:1_18:3 | | | | | | | | | |
| PC 18:1_20:0 | 2.1610547 | 1.8750072 | 1.5194017 | 2.0120436 | 1.2968441 | 2.6378534 | 1.9286398 | 1.6780902 | 2.2270545 |
| PC 18:1_20:1 | | 0.4718633 | 0.4332295 | | | 0.7337087 | | | |
| PC 18:1_20:2 | | | 0.5384662 | 0.3761319 | 0.3069766 | 0.8245082 | | | 0.4562686 |
| PC 18:1_20:3 | 4.4822126 | 4.2987855 | 3.8056748 | 3.9522085 | 4.8178336 | 6.4704668 | 4.1484012 | 3.6547625 | 6.2330707 |
| PC 18:1_20:4 | 17.009433 | 14.391104 | 21.609525 | 18.593684 | 17.741196 | 22.745374 | 15.719424 | 15.139215 | 24.583303 |
| PC 18:1_20:5 | | | 3.5063687 | 3.3348126 | 1.825129 | 3.5122328 | | 2.6963802 | 5.8428591 |
| PC 18:1_22:5 | | | | | | | | | |
| PC 18:1_22:6 | 16.596759 | 18.282796 | 15.188555 | 18.457246 | 12.387177 | 24.759821 | 16.417923 | 18.772914 | 26.836021 |
| PC 18:2_18:2 | 13.904155 | 11.477931 | 6.1220212 | 15.983529 | 7.2586799 | 11.714548 | 12.660191 | 9.952324 | 8.4140665 |
| PC 18:2_18:3 | 1.0345669 | | | 1.9130952 | | | | | |
| PC 18:2_20:0 | 10.610847 | 9.7997463 | 5.2704961 | 10.695468 | 4.7410739 | 9.0988478 | 11.196696 | 8.2020042 | 6.2650265 |
| PC 18:2_20:1 | 2.1477282 | 1.9306927 | 1.1216934 | 2.0422742 | 0.9715457 | 1.4104702 | 2.1906743 | 1.3961018 | 1.0852024 |
| PC 18:2_20:2 | 1.0604397 | 0.806984 | 0.44922 | 0.8303167 | 0.5005576 | 0.9773994 | | 0.7913684 | 0.7127922 |
| PC 18:2_20:3 | 2.1369208 | 1.811133 | 1.2668275 | 1.9786685 | 1.1834923 | 2.6199001 | 2.0755734 | 1.6922532 | 2.1042944 |
| PC 18:2_20:4 | 6.2442814 | 4.7554584 | 4.5617017 | 9.4998349 | 4.0369491 | 6.1711988 | 6.9933756 | 5.7628693 | 5.0029924 |
| PC 18:2_20:5 | | 1.7966727 | | | | | | | |
| PC 18:2_22:6 | | | | 5.2301277 | | | | 4.6286618 | |
| PC 20:0_20:3 | | | | 0.9212872 | | | 0.8158567 | | |
| PC 20:0_20:4 | 3.7840642 | 2.8148862 | 2.6339747 | 3.9682591 | 2.232221 | 3.5966626 | 3.3142215 | 2.8090413 | 2.1184958 |
| PC 20:0_22:6 | | | | 3.5644498 | | | 4.0009885 | | |
| PE 16:0_18:1 | 1.1417238 | | | | 0.8169335 | | | | |
| PE 16:0_18:2 | 5.3968251 | | | | 5.736519 | | | | |
| PE 16:0_20:4 | 5.362230 | | | | 5.2177041 | | | | |
| PE 18:0_18:0 | 0.8095919 | | | | 0.8044152 | | | 1.8901686 | 1.145921 |
| PE 18:0_18:1 | 29.51101 | | | | 30.83376 | | | 107.0333 | 76.636298 |
| PE 18:0_18:2 | 109.70253 | | | | 191.16627 | | | | |
| PE 18:0_20:1 | 1.5702127 | | | | | | | 3.1961115 | 4.6203976 |
| PE 18:0_20:2 | 2.4810922 | | | | 3.1289758 | | | | |
| PE 18:0_20:4 | 42.689284 | | | | 58.757719 | | | | |
| PE 18:0_22:2 | | | | | | | | | |
| PE 18:0_22:4 | 1.1253272 | | | | | | | | |
| PE 18:1_18:1 | 0.5278017 | | | | 0.5965006 | | | | 1.042178 |
| PE 18:1_18:2 | 3.4680553 | | | | 5.0700923 | | | | |
| PE 18:1_20:4 | 3.3713824 | | | | 4.5249556 | | | | |
| PE 18:1_22:0 | 1.7109477 | | | | 1.0034041 | | | 4.850343 | 4.540962 |
| PE 18:2_18:2 | 0.2482566 | | | | | | | | |
| PE O-16:0/18:1 | | | | | | | | | |
| PE O-16:0/18:2 | 0.7526089 | | | | | | | | |
| PE O-16:0/20:4 | | | | | | | | | |
| PE O-18:0/16:0 | 0.7979952 | | | | | | | | 1.5469198 |
| PE O-18:0/18:1 | 1.0921612 | | | | 0.70847 | | | 1.7676861 | |
| PE O-18:0/18:2 | 3.4293909 | | | | 4.9686699 | | | | |
| PE O-18:0/20:4 | 3.1922201 | | | | 4.0932839 | | | | |
| PE P-16:0/16:0 | 0.3613248 | | | | | | | | |
| PE P-16:0/18:1 | 0.4907919 | | | | | | | | |
| PE P-16:0/18:2 | 0.9410121 | | | | 1.5459661 | | | | |
| PE P-16:0/20:4 | 2.4403893 | | | | 2.6842913 | | | | |
| PE P-18:0/16:0 | 0.847506 | | | | 0.6704668 | | | | 1.8016999 |
| PE P-18:0/18:1 | 0.7186229 | | | | | | | 1.6609995 | |
| PE P-18:0/18:2 | 2.0318167 | | | | 2.4634018 | | | | |
| PE P-18:0/20:4 | 3.6852475 | | | | 4.9816537 | | | | |
| PE P-18:1/18:1 | 0.2215604 | | | | | | | | |
| PE P-18:1/18:2 | 0.3486617 | | | | | | | | |
| PE P-18:1/20:4 | 0.6580275 | | | | | | | | |
| PG16:0_18:1 | 0.1693643 | 0.2076305 | | 0.1485438 | 0.125759 | 0.1584124 | 0.1436004 | 0.1103193 | 0.1063306 |
| PG16:0_18:2 | 0.1667104 | 0.2485909 | 0.1008355 | 0.222246 | 0.1591537 | | 0.136307 | 0.1145947 | 0.1339546 |
| PG16:1_18:1 | 0.1669876 | 0.1154631 | 0.1525019 | 0.1584127 | 0.0913123 | 0.1876074 | 0.127165 | | |
| PG16:1_18:2 | 0.89022 | 0.6049412 | 0.4659629 | 0.6796558 | 0.3768401 | 0.552141 | 0.6018899 | 0.4583627 | 0.4270092 |
| PG17:0_20:0 | 0.6620726 | 0.6560983 | 0.5360821 | 0.5001487 | 0.6118173 | 0.5162376 | 0.6540045 | 0.5901231 | 0.5908622 |
| PG17:0_22:1 | | | | | | | | | |
| PG18:0_18:0 | 0.1412476 | | | | | | | | |
| PG18:0_18:1 | | 0.2361327 | 0.1329718 | 0.1984617 | | 0.1440503 | 0.1263403 | 0.1448448 | 0.1783942 |
| PG18:0_18:2 | 0.4766788 | 1.0065034 | 0.7426733 | 1.2034721 | 0.4691767 | 0.5468217 | 0.9249107 | 0.7326062 | 0.7064147 |
| PG18:0_20:0 | 0.3574497 | 0.5695422 | 0.4418251 | 0.7591724 | 0.3609737 | 0.3635171 | 0.6173787 | 0.8075624 | 0.3965746 |
| PG18:0_20:4 | 0.6585933 | 1.934068 | 0.9280979 | 1.8932769 | 0.848146 | 1.3606769 | 1.2296107 | 1.1475034 | 0.8619081 |
| PG18:0_22:5 | | | | | | | | | |
| PG18:0_22:6 | 0.5980578 | 1.6406873 | 0.6072998 | 1.987188 | 0.7955585 | 1.1248437 | 0.8485643 | 1.2654647 | 0.5976936 |
| PG18:1_18:1 | | 0.07716 | 0.0698412 | 0.0686065 | | | 0.0584884 | | 0.063299 |
| PG18:1_18:2 | 0.677849 | 1.0660835 | 0.6199986 | 0.9684384 | 0.4289634 | 0.6668506 | 0.7417731 | 0.6582767 | 0.6091889 |
| PG18:1_20:0 | 0.1318641 | 0.1905125 | 0.1590766 | 0.1734277 | | 0.1507497 | | 0.1272672 | 0.1321904 |
| PG18:1_20:3 | | | | | | 0.1750502 | 0.1705753 | | |
| PG18:1_20:4 | 0.9744638 | 1.7990793 | 0.822709 | 2.0117435 | 0.97045 | 1.2002853 | 1.1208615 | 1.352681 | 0.8327936 |
| PG18:1_22:6 | | 0.5853597 | | 0.8468339 | | | 0.4530363 | | |
| PG18:2_18:2 | 0.1028302 | 0.103843 | 0.082801 | 0.1346842 | | 0.0861313 | 0.0774255 | 0.0721479 | 0.1044453 |
| PG18:2_20:0 | 0.6499151 | 1.9118394 | 0.9730957 | 1.7126021 | 0.8493149 | 0.7254455 | 1.1853477 | 1.0700593 | 0.7306178 |
| PG18:2_20:4 | 0.3462478 | 0.5626845 | | 0.6829957 | | | 0.3453305 | | |
| PG20:0_20:3 | | 0.2651261 | | 0.2753035 | 0.1247889 | | 0.2740811 | 0.2407703 | 0.1407383 |
| PG20:0_20:4 | 1.3724666 | 5.2870069 | 2.5965476 | 6.0728223 | 2.1875282 | 2.6985337 | 3.5471405 | 3.7045555 | 2.3304141 |
| PG20:0_20:5 | | | | | | | | | |
| PG20:0_22:6 | | 0.9771695 | 0.4357807 | 1.3683572 | | | 0.7867443 | 0.8571132 | |
| PI 14:0_18:1 | 0.4482252 | 1.2756011 | 1.2364706 | 1.3950149 | 0.8195986 | 1.1826703 | 1.2221545 | 1.1886726 | 1.2613442 |
| PI 14:0_18:2 | | 0.5266699 | 0.4218556 | 0.6400944 | 0.3594788 | 0.3387243 | 0.3915612 | 0.5107716 | |
| PI 15:0_18:1 | | | 0.1938417 | | | | | | |
| PI 15:0_20:1 | | 0.2791117 | | | | | | | |
| PI 15:0_20:2 | 0.5740008 | 0.5286127 | 0.2876439 | 0.4702941 | | 0.4454927 | 0.5750447 | 0.3525391 | 0.319505 |
| PI 15:0_22:0 | 0.9820666 | 0.9041702 | 0.696168 | 1.0152911 | 0.8968355 | 0.8241674 | 0.9291576 | 0.9915339 | 0.8212537 |
| PI 15:0_22:1 | 0.3761289 | 0.2795974 | 0.331728 | | | 0.4128059 | 0.3352466 | | |
| PI 15:0_22:2 | 0.826305 | 0.531656 | 0.4874384 | | | 0.696532 | | | |
| PI 15:0_22:4 | 0.982108 | 0.9060597 | 0.667553 | 0.8053208 | 0.669322 | 0.9731621 | 1.0075072 | 1.0675877 | 0.8831955 |
| PI 15:0_22:6 | | | | | | 0.4940149 | | | |
| PI 16:0_16:0 | 0.1444704 | 0.1570128 | | | 0.1205382 | | | | |
| PI 16:0_18:0 | | 0.2409552 | | | | | | | |
| PI 16:0_18:1 | 2.1493831 | 1.8736974 | 1.2283189 | 1.774652 | 1.0921982 | 1.3733989 | 1.7975194 | 1.2234567 | 1.0993522 |
| PI 16:0_18:2 | 10.479836 | 8.4896564 | 5.2439698 | 9.0032899 | 4.7232612 | 8.0333692 | 14.100062 | 7.9239609 | 5.677306 |
| PI 16:0_20:2 | | | | | | | 0.4249905 | | |
| PI 16:0_20:3 | 3.1130446 | 2.643188 | 2.2393512 | 2.4509149 | 1.2951927 | 3.9908452 | 4.6399219 | 2.7629671 | 2.2355459 |
| PI 16:0_20:4 | 11.878469 | 8.9718084 | 8.429902 | 10.713773 | 6.6351448 | 9.6144432 | 15.833689 | 12.577918 | 9.1086581 |
| PI 16:0_20:5 | | | | | | | | | |
| PI 16:0_22:5 | | | | 1.0192146 | | 0.9946676 | | | |
| PI 16:0_22:6 | | | | | | | 0.9102115 | | |
| PI 17:0_18:1 | 0.4220351 | | | | | 0.2616165 | 0.2042025 | | 0.2404196 |
| PI 17:0_18:2 | 0.8673851 | 0.773664 | | 0.5547564 | 0.372481 | 0.483138 | 0.7784413 | 0.5781269 | 0.4136156 |
| PI 17:0_20:0 | 1.2929791 | 1.3631242 | 1.4172554 | 1.6005726 | 1.5701538 | 1.3015584 | 1.5081555 | 1.4538939 | 0.9434704 |
| PI 17:0_20:1 | | | 0.3174437 | | | | | | |
| PI 17:0_20:3 | | | | | | | | 0.3503346 | |
| PI 17:0_20:4 | 1.2258442 | 1.5370822 | 1.4577843 | 1.9765738 | 1.1212775 | 1.6352356 | 2.0678273 | 1.6909443 | 1.9039578 |
| PI 17:0_22:1 | 0.8752914 | 0.9453257 | 0.9792074 | 1.0678315 | 0.8404609 | 1.2229358 | 1.0465978 | 1.1893788 | 1.0004493 |
| PI 17:0_22:2 | | | | | | 0.7007332 | | | |
| PI 18:0_18:0 | 0.0878502 | 0.1047747 | | 0.1076935 | | | | | 0.07867 |
| PI 18:0_18:1 | 2.2844416 | 2.0660663 | 0.9676869 | 1.757728 | 1.1483175 | 1.2821469 | 1.6515716 | 1.1471548 | 1.2429826 |
| PI 18:0_18:2 | 16.695923 | 14.857465 | 7.0892136 | 13.462318 | 7.9633142 | 11.366838 | 17.066721 | 10.596106 | 7.7683361 |
| PI 18:0_20:0 | 0.8575562 | 0.7046491 | 0.6382353 | 0.5190382 | | 0.803704 | 0.8243041 | 0.4993139 | 0.6241641 |
| PI 18:0_20:2 | 0.360901 | 0.3741344 | | 0.2939812 | | 0.4504558 | 0.5179457 | 0.261804 | 0.1947127 |
| PI 18:0_20:3 | 9.914802 | 7.1818413 | 8.9432577 | 8.7048366 | 4.8822396 | 12.502167 | 13.319612 | 9.0781611 | 10.266198 |
| PI 18:0_20:4 | 95.968014 | 89.87959 | 114.18543 | 112.12407 | 71.730313 | 101.70869 | 159.02277 | 154.06226 | 148.82018 |
| PI 18:0_20:5 | 1.5031693 | 1.1322582 | 1.1923019 | 1.6284172 | | 1.050388 | 1.8063181 | 1.884107 | 2.2659987 |
| PI 18:0_22:4 | | | | | | | | | |
| PI 18:0_22:5 | 1.3738957 | | | 1.444313 | | 1.8532739 | 1.1957084 | 0.8709617 | 1.2361753 |
| PI 18:0_22:6 | 1.6814039 | 1.2623365 | 1.2317289 | 2.4328763 | 0.7169748 | 1.6104206 | 2.6667573 | 1.6048492 | 2.3750762 |
| PI 18:1_18:1 | 0.5491862 | 0.4843313 | 0.2500876 | 0.5201405 | 0.2941515 | 0.3880064 | 0.6036555 | 0.4267206 | 0.3183867 |
| PI 18:1_18:2 | 2.1644557 | 1.7254539 | 0.9383775 | 2.6739068 | 1.2818394 | 2.1455805 | 2.5172453 | 1.7032011 | 1.5549687 |
| PI 18:1_20:0 | | | | | | | | | |
| PI 18:1_20:3 | 0.5448613 | | | 0.384919 | | 0.8656771 | 0.6811497 | 0.4512296 | |
| PI 18:1_20:4 | 3.5896363 | 2.5445041 | 2.0676146 | 3.9527436 | 1.9346434 | 4.4410561 | 4.370778 | 3.7303713 | 6.1309085 |
| PI 18:2_18:2 | 0.2171772 | | | 0.2699076 | | 0.1925706 | 0.3400813 | | |
| PI 18:2_20:0 | | | | | | | | | |
| PI 20:0_20:4 | 1.1764133 | | | 0.9768478 | | | 0.8542495 | 0.9136481 | |
| PS 16:0_18:2 | | | | | | | | 0.9707876 | |
| PS 16:0_22:6 | | | | | | | | | |
| PS 16:1_18:1 | | | | 1.9924702 | | | | | |
| PS 16:1_18:2 | 1.5124787 | 4.8231702 | 2.7012247 | 4.1971221 | 1.0142315 | 4.0212022 | 2.911965 | 3.8071667 | |
| PS 17:0_20:0 | 1.3133503 | 0.9197031 | 0.7848314 | 1.3257164 | 1.1983263 | 1.1586325 | 1.2849639 | 1.338248 | 1.1721793 |
| PS 17:0_20:3 | 0.9616992 | 1.6396155 | 2.5181126 | 2.103111 | 1.0171948 | 2.8804058 | 1.8831001 | 1.720817 | 2.1162176 |
| PS 17:0_22:1 | | | | | 0.680698 | | | | |
| PS 18:0_18:0 | 0.2423458 | 0.4776671 | | 0.6601752 | | | 0.39919 | | |
| PS 18:0_18:1 | 0.6252377 | 2.9668132 | 1.856358 | 2.4532666 | | 2.1723169 | 1.8217894 | 2.5118505 | 1.7084713 |
| PS 18:0_18:2 | 1.3639859 | 7.8814894 | 5.6443717 | 8.6298999 | 1.7737574 | 4.6626889 | 5.5173604 | 6.7734019 | 4.3249796 |
| PS 18:0_20:0 | 0.575409 | 2.5796642 | 1.2964207 | 3.1234163 | 0.5451507 | 1.1057392 | 1.7594381 | 2.2343676 | 1.1086999 |
| PS 18:0_20:4 | | | | | 3.4781261 | | | | |
| PS 18:0_22:5 | | | | | | | | | |
| PS 18:0_22:6 | 3.3400542 | | | | 6.5439021 | | | | |
| PS 18:1_18:1 | | 1.4338673 | | 1.0782154 | | 0.83822 | 0.7743528 | 1.1349326 | 0.880352 |
| PS 18:1_18:2 | 2.0754907 | 9.7754247 | 5.4270699 | 7.1723633 | 1.8265695 | 4.9343449 | 5.0908436 | 9.3346547 | 4.1991042 |
| PS 18:1_20:0 | 0.8990573 | 4.4432149 | 3.5763001 | 5.2226995 | 1.1852159 | 4.182646 | 3.1324792 | 5.6846814 | 3.0396276 |
| PS 18:1_20:4 | | | | | 4.6629414 | | | | |
| PS 18:1_22:5 | | | | | | | | | |
| PS 18:1_22:6 | 3.3881406 | | | | 7.0105477 | | | | |
| PS 18:2_18:2 | 0.3737647 | | 0.7724896 | 1.3772976 | | | | | |
| PS 18:2_20:0 | 1.8509246 | 11.399639 | 6.0486942 | 10.63564 | 2.9075263 | 6.2183357 | 8.6595042 | 10.769662 | 7.2129034 |
| PS 20:0_20:4 | 1.7598494 | | | | 4.1127409 | | | | |
| PS 20:0_22:6 | | | | | 4.045929 | | | | |
| SM d18:1/14:0 | 0.9993765 | 1.0398783 | 1.1385214 | 0.9328712 | 0.6592949 | 1.0906381 | 1.0205519 | 0.8668596 | 1.3147461 |
| SM d18:1/16:0 | 34.869876 | 38.191305 | 29.739705 | 34.497489 | 31.143308 | 28.386125 | 38.519616 | 34.478444 | 39.957699 |
| SM d18:1/18:0 | 3.7939545 | 4.01767 | 4.1169515 | 3.5291281 | 4.0380513 | 3.2350868 | 3.3389482 | 2.7969878 | 5.2054917 |
| SM d18:1/18:1 | 1.8001195 | 1.8276435 | 1.7695944 | 1.5144783 | 1.6220462 | 1.5588204 | 1.4544809 | 1.1748554 | 2.0878258 |
| SM d18:1/20:0 | 37.65012 | 34.622669 | 28.980684 | 35.70145 | 25.910534 | 36.282235 | 44.835776 | 36.61861 | 32.114441 |
| SM d18:1/20:1 | 1.8660219 | 1.9115481 | 2.0143407 | 2.0464032 | 1.1826062 | 2.1776245 | 1.9143468 | 1.7028057 | 1.8904184 |
| SM d18:1/22:0 | 44.227358 | 45.098631 | 30.438054 | 48.911633 | 29.587371 | 36.580506 | 52.779384 | 45.550604 | 34.741739 |
| SM d18:1/22:1 | 14.008544 | 14.273929 | 9.1861414 | 15.077161 | 8.8151413 | 13.802555 | 14.825387 | 12.220575 | 10.217126 |
| SM d18:1/24:0 | 13.475545 | 13.549401 | 10.226827 | 15.186201 | 7.9832295 | 15.069362 | 16.356129 | 13.810742 | 11.636069 |
| SM d18:1/24:1 | 23.760403 | 24.693226 | 35.476848 | 26.706506 | 32.492031 | 33.130793 | 30.726971 | 24.512321 | 41.862909 |
| SM d18:1/26:0 | 2.0671055 | 2.008818 | 2.7703769 | 1.7248993 | 1.2274737 | 2.9296086 | 2.0599659 | 1.6178563 | 2.8122598 |
| SM d18:1/26:1 | 3.4291923 | 2.9535914 | 3.9041673 | 2.9277254 | 2.0687725 | 3.7602491 | 3.373527 | 2.7283356 | 3.9898355 |
| TG 36:0-FA12:0 | | | | 0.0654359 | | | | | |
| TG 38:0-FA12:0 | | 0.0525861 | | | | | | | |
| TG 40:0-FA12:0 | | | | | | | | | |
| TG 40:0-FA16:0 | | 0.1139181 | 0.0685049 | | | 0.0763857 | | 0.0396226 | |
| TG 42:0-FA16:0 | 0.1297091 | 0.1178644 | 0.1113497 | | 0.1007035 | 0.074922 | | 0.044709 | 0.0768393 |
| TG 42:1-FA16:0 | 0.219444 | 0.2723216 | 0.2214168 | 0.1407811 | 0.1445329 | 0.1847786 | | 0.065495 | 0.1571259 |
| TG 42:1-FA16:1 | | 0.043688 | | | | | | | |
| TG 42:1-FA18:1 | 0.1294847 | 0.161164 | 0.0920238 | 0.0954678 | 0.0837731 | 0.0945976 | | | |
| TG 42:2-FA18:2 | 0.1504308 | 0.1761552 | 0.1319307 | 0.1444596 | 0.0789114 | 0.1555128 | | 0.0494406 | 0.1136016 |
| TG 44:0-FA12:0 | | 0.0759014 | | | | | | | |
| TG 44:0-FA14:0 | | | | | | | | | |
| TG 44:0-FA16:0 | 0.1373076 | 0.1123753 | 0.0992517 | 0.0780975 | 0.1216646 | 0.0928709 | | 0.0420655 | 0.091487 |
| TG 44:0-FA18:0 | | | | | | | | | |
| TG 44:1-FA14:0 | | | | | | | | | |
| TG 44:1-FA16:0 | 0.3917805 | 0.3258881 | 0.3314145 | 0.2448764 | 0.2023017 | 0.1949871 | 0.0718364 | 0.0971629 | 0.2177894 |
| TG 44:1-FA18:1 | 0.2330254 | 0.1734814 | 0.2119021 | 0.1201353 | 0.144889 | 0.1076196 | | | 0.1194862 |
| TG 44:2-FA16:0 | 0.5321959 | 0.4438073 | 0.4183844 | 0.4056077 | 0.253031 | 0.290443 | 0.1313924 | 0.1340969 | 0.3027262 |
| TG 44:2-FA16:1 | | | | | | | | | |
| TG 44:2-FA18:1 | 0.1002235 | 0.1426105 | 0.1151046 | 0.0983322 | 0.1190082 | 0.1139609 | | | |
| TG 44:2-FA18:2 | 0.3113861 | 0.2196385 | 0.1821682 | 0.2378834 | 0.0852946 | 0.1492738 | 0.0753891 | 0.072418 | 0.1546914 |
| TG 44:3-FA18:2 | 0.1762771 | 0.1339955 | 0.1349222 | 0.17131 | 0.1001802 | 0.1123487 | | 0.0628604 | 0.1211179 |
| TG 45:0-FA16:0 | 0.1764751 | 0.1891155 | 0.1723448 | 0.1051783 | 0.0946664 | 0.0945794 | | 0.0393235 | 0.1008726 |
| TG 45:1-FA16:0 | 0.3422002 | 0.3405265 | 0.3519845 | 0.3022069 | 0.1470092 | 0.2214947 | | | 0.2685366 |
| TG 45:1-FA18:1 | 0.1427185 | 0.1747835 | 0.1244083 | 0.1054794 | 0.0867114 | 0.1103588 | | | 0.1001569 |
| TG 46:0-FA12:0 | | 0.0188204 | | | | | | | |
| TG 46:0-FA14:0 | 0.1441458 | 0.3416178 | 0.1174405 | 0.0959742 | 0.2505749 | 0.0785441 | | | 0.079324 |
| TG 46:0-FA16:0 | 0.3583508 | 0.6770417 | 0.2391886 | 0.2161433 | 0.5690048 | 0.2046641 | 0.1022079 | 0.1386451 | 0.2064385 |
| TG 46:1-FA12:0 | 0.1812865 | 0.3353912 | 0.2269384 | 0.1862389 | 0.2089272 | 0.134479 | 0.0808734 | 0.0938203 | 0.1479049 |
| TG 46:1-FA14:0 | 0.1042023 | 0.2251789 | 0.0979177 | 0.0741635 | 0.4163679 | | | | |
| TG 46:1-FA14:1 | 0.0671574 | 0.1084214 | 0.0918377 | | | | | | |
| TG 46:1-FA16:0 | 0.459347 | 0.4077429 | 0.3216662 | 0.3290226 | 0.4733926 | 0.1807162 | 0.0955533 | 0.1465118 | 0.1784064 |
| TG 46:1-FA16:1 | 0.0760867 | 0.1620882 | | | 0.2288306 | | | 0.0553183 | 0.0738789 |
| TG 46:1-FA18:0 | 0.0757591 | 0.0631355 | | | | | | | |
| TG 46:1-FA18:1 | 0.1268022 | 0.1267111 | 0.0987503 | 0.0899712 | 0.1355713 | | | | 0.066477 |
| TG 46:2-FA12:0 | 0.4043214 | 0.462678 | 0.3722513 | 0.5443101 | 0.269167 | 0.297199 | 0.186059 | 0.310203 | 0.2659397 |
| TG 46:2-FA14:0 | 0.0915075 | 0.1860228 | 0.0557306 | 0.0924357 | 0.2392594 | | | | |
| TG 46:2-FA14:1 | | | | | 0.0861406 | | | | |
| TG 46:2-FA16:0 | 0.2975112 | 0.3449468 | 0.3267521 | 0.2874364 | 0.2804474 | 0.2192219 | 0.1130917 | 0.1939097 | 0.2220612 |
| TG 46:2-FA16:1 | | 0.071748 | | | 0.1736609 | | | | |
| TG 46:2-FA18:1 | 0.2158867 | 0.2517339 | 0.1674785 | 0.1460454 | 0.210925 | 0.1456551 | | 0.0737394 | 0.1603307 |
| TG 46:2-FA18:2 | 0.1714222 | 0.2040687 | 0.1297138 | 0.1808282 | 0.19879 | 0.0906908 | 0.0943251 | 0.1199915 | 0.0954149 |
| TG 46:3-FA12:0 | 0.0765029 | 0.1170348 | 0.1022393 | 0.1107576 | | 0.0776156 | 0.0724944 | | 0.0586901 |
| TG 46:3-FA14:1 | | | | | | | | | |
| TG 46:3-FA16:0 | 0.4229518 | 0.3914254 | 0.3373896 | 0.385865 | 0.2152134 | 0.2567284 | 0.1021225 | 0.1811726 | 0.2435641 |
| TG 46:3-FA16:1 | | | | | 0.0742999 | 0.0663381 | | | |
| TG 46:3-FA18:1 | 0.2720723 | 0.2370481 | 0.201871 | 0.1544879 | 0.1731438 | 0.144012 | 0.0634237 | 0.0618317 | 0.1519463 |
| TG 46:3-FA18:2 | 0.3226704 | 0.2466315 | 0.2307267 | 0.2945007 | 0.1500583 | 0.1672243 | 0.0862868 | 0.124388 | 0.2131068 |
| TG 46:3-FA18:3 | | | | 0.0589516 | | | | | |
| TG 46:4-FA18:2 | 0.2654947 | 0.2664548 | 0.1456473 | 0.2523443 | 0.1459939 | 0.1507081 | 0.0910469 | 0.0936337 | 0.1437477 |
| TG 47:0-FA14:0 | | 0.0551229 | | | | | | | |
| TG 47:0-FA15:0 | 0.289562 | 0.7072061 | 0.1731099 | 0.1238603 | 0.3069852 | 0.1393496 | 0.1274257 | 0.1246439 | 0.2063207 |
| TG 47:0-FA16:0 | 0.7308895 | 1.662377 | 0.3646042 | 0.4037451 | 0.7294404 | 0.3785637 | 0.2800475 | 0.2555864 | 0.3750349 |
| TG 47:0-FA17:0 | 0.0689932 | | | | | | | | |
| TG 47:1-FA14:0 | | 0.0911324 | | | | | | | |
| TG 47:1-FA15:0 | 0.1256706 | 0.2386102 | 0.0810789 | | 0.1247302 | 0.0662982 | | | 0.0706723 |
| TG 47:1-FA16:0 | 0.3633394 | 0.5626882 | 0.2624998 | 0.2966751 | 0.2590986 | 0.1936208 | 0.1063523 | 0.1304063 | 0.1712996 |
| TG 47:1-FA16:1 | 0.1383728 | 0.2269611 | 0.1015693 | 0.0835477 | 0.139298 | | | 0.0600491 | 0.0790713 |
| TG 47:1-FA17:0 | | 0.0565174 | | | | | | | |
| TG 47:1-FA18:1 | 0.1261135 | 0.1575263 | 0.0908777 | 0.0806231 | 0.0994735 | 0.0705052 | 0.0553943 | | 0.1076564 |
| TG 47:2-FA14:0 | 0.0746359 | 0.1340601 | | | 0.0736706 | | | | |
| TG 47:2-FA15:0 | 0.0820559 | 0.1648785 | | | 0.0873134 | | | | |
| TG 47:2-FA16:1 | | 0.0708823 | 0.065511 | | 0.0723064 | | | | |
| TG 47:2-FA18:1 | 0.1857605 | 0.1494693 | 0.1241262 | 0.0992244 | 0.13479 | 0.1144508 | | 0.0523303 | 0.1053969 |
| TG 47:2-FA18:2 | 0.2106392 | 0.2912339 | 0.1846377 | 0.1951955 | | 0.1080924 | 0.0845058 | 0.0947603 | 0.1375082 |
| TG 48:0-FA14:0 | 0.0552307 | 0.2054076 | 0.0674788 | 0.0474766 | 0.0558711 | 0.0420689 | 0.0541532 | 0.0483308 | 0.0507646 |
| TG 48:0-FA16:0 | 3.9485906 | 8.2115884 | 2.4008179 | 2.1053726 | 7.6717947 | 2.272737 | 0.8635929 | 1.1643249 | 2.1744401 |
| TG 48:0-FA18:0 | 0.0862898 | 0.1758155 | 0.0455301 | | 0.1091294 | 0.0657765 | | 0.052545 | |
| TG 48:1-FA12:0 | | 0.099729 | 0.0634193 | 0.0424967 | 0.0592004 | 0.0559639 | | 0.0389622 | 0.053434 |
| TG 48:1-FA14:0 | 0.7162274 | 1.6485116 | 0.6205012 | 0.600609 | 2.4734004 | 0.4256515 | 0.177728 | 0.3652272 | 0.4404299 |
| TG 48:1-FA14:1 | | 0.0414524 | 0.0311841 | | 0.0409787 | 0.0320124 | | | 0.0314268 |
| TG 48:1-FA16:0 | 1.9452686 | 4.0433162 | 1.7451253 | 1.5028361 | 6.2062051 | 1.1208809 | 0.4403927 | 1.1175672 | 1.260267 |
| TG 48:1-FA16:1 | 0.71905 | 1.3725688 | 0.6386837 | 0.4377349 | 1.8270001 | 0.4261376 | 0.1582044 | 0.3430394 | 0.5396712 |
| TG 48:1-FA18:0 | | 0.1079671 | 0.0651113 | 0.0415149 | 0.057967 | | | | |
| TG 48:1-FA18:1 | 0.8307162 | 1.5543967 | 0.6223605 | 0.6475122 | 2.4873715 | 0.3643414 | 0.2000306 | 0.3570876 | 0.4422836 |
| TG 48:2-FA12:0 | 0.1732923 | 0.36279 | 0.245643 | 0.3316936 | 0.2227908 | 0.1714558 | 0.1901899 | 0.1739268 | 0.1632616 |
| TG 48:2-FA14:0 | 1.5957487 | 3.0673988 | 1.2734297 | 1.9851866 | 4.2413878 | 0.9064424 | 0.5070281 | 0.9250909 | 0.8359406 |
| TG 48:2-FA14:1 | 0.3602779 | 0.4806583 | 0.4179452 | 0.4074977 | 0.4385837 | 0.3556391 | 0.1144468 | 0.1520788 | 0.3353055 |
| TG 48:2-FA16:0 | 1.852795 | 3.2039314 | 1.4770517 | 2.0594949 | 4.8651635 | 0.9501145 | 0.4974965 | 1.0969339 | 0.8813328 |
| TG 48:2-FA16:1 | 0.5552692 | 1.0425732 | 0.6406021 | 0.4972223 | 3.6958512 | 0.2143962 | 0.0848409 | 0.2487348 | 0.3954042 |
| TG 48:2-FA18:0 | | | | | 0.0469238 | | | | |
| TG 48:2-FA18:1 | 0.3845697 | 0.5588091 | 0.3943248 | 0.3832979 | 1.5124208 | 0.2249108 | 0.0822211 | 0.1691922 | 0.2462326 |
| TG 48:2-FA18:2 | 1.4962061 | 2.453902 | 0.9516671 | 1.6199818 | 2.886835 | 0.6219341 | 0.4568661 | 0.8041091 | 0.5925804 |
| TG 48:3-FA12:0 | 0.2349571 | 0.2557866 | 0.2463052 | 0.3666906 | 0.2628666 | 0.156383 | 0.1654355 | 0.1583307 | 0.189753 |
| TG 48:3-FA14:0 | 0.3589712 | 0.6660057 | 0.3195223 | 0.5559995 | 1.5361532 | 0.1770942 | 0.0909576 | 0.1447732 | 0.178992 |
| TG 48:3-FA14:1 | 0.7436727 | 0.8604336 | 0.768873 | 1.0070561 | 0.6250456 | 0.595707 | 0.3221858 | 0.4442867 | 0.5307333 |
| TG 48:3-FA16:0 | 0.5028334 | 0.6069152 | 0.4300581 | 0.6227949 | 0.7122561 | 0.2510869 | 0.1496551 | 0.2571958 | 0.2877473 |
| TG 48:3-FA16:1 | 0.3901132 | 0.744117 | 0.3247397 | 0.4549509 | 1.7167365 | 0.1355435 | 0.0971634 | 0.1766788 | 0.1859249 |
| TG 48:3-FA18:1 | 0.2685913 | 0.2393855 | 0.2571535 | 0.3008849 | 0.3371438 | 0.1389921 | 0.1078954 | 0.1418549 | 0.1995268 |
| TG 48:3-FA18:2 | 0.5645921 | 0.7872708 | 0.5083136 | 0.7855489 | 1.4402471 | 0.2892041 | 0.2102336 | 0.3654474 | 0.3457185 |
| TG 48:3-FA18:3 | 0.139047 | 0.2153217 | 0.0962672 | 0.159632 | 0.2530507 | 0.071502 | 0.0596925 | | 0.0914637 |
| TG 48:4-FA12:0 | 0.1743581 | 0.2056021 | 0.1646782 | 0.36173 | 0.1676902 | 0.1541717 | 0.2074258 | 0.183994 | 0.1658474 |
| TG 48:4-FA14:0 | | | | 0.0969229 | 0.0903955 | | | | |
| TG 48:4-FA14:1 | 0.1154936 | 0.110589 | 0.1139127 | 0.1331824 | 0.0969459 | 0.0780187 | | | 0.1010879 |
| TG 48:4-FA16:0 | 0.2565076 | 0.2756937 | 0.2180025 | 0.3124032 | 0.196512 | 0.1404336 | 0.1058887 | 0.1428206 | 0.1845644 |
| TG 48:4-FA16:1 | 0.0855025 | 0.0981283 | 0.0731903 | 0.0840383 | 0.1676902 | | | | |
| TG 48:4-FA18:1 | 0.179982 | 0.1551928 | 0.1491224 | 0.1711448 | 0.1432363 | 0.0966586 | 0.0547356 | | 0.1076921 |
| TG 48:4-FA18:2 | 0.3744723 | 0.2923325 | 0.2994386 | 0.6623428 | 0.278852 | 0.1893407 | 0.1589458 | 0.2235882 | 0.2129653 |
| TG 48:4-FA18:3 | | | 0.0791365 | 0.0849745 | 0.1338923 | | | | |
| TG 48:5-FA18:2 | 0.2452278 | 0.2102118 | 0.1207278 | 0.2580663 | 0.1401365 | 0.1188669 | 0.0671623 | 0.0744692 | 0.1152688 |
| TG 48:5-FA18:3 | | | | 0.0706806 | | | | | |
| TG 49:0-FA15:0 | 0.1823326 | 0.3637087 | 0.061299 | 0.0701439 | 0.1502933 | 0.1001131 | 0.0826393 | 0.0440121 | 0.1190868 |
| TG 49:0-FA16:0 | 0.8264445 | 2.1964449 | 0.5057289 | 0.7335947 | 1.2217891 | 0.4807038 | 0.3715407 | 0.3350866 | 0.5306922 |
| TG 49:0-FA17:0 | 0.2810993 | 0.7796322 | 0.2190268 | 0.2098807 | 0.4421893 | 0.2275348 | 0.1591298 | 0.1280073 | 0.2790563 |
| TG 49:0-FA18:0 | 0.19965 | 0.5100091 | 0.156204 | 0.1706235 | 0.1962323 | 0.1607143 | 0.1354145 | 0.1230254 | 0.1679639 |
| TG 49:1-FA14:0 | 0.1089882 | 0.1256527 | 0.0680693 | 0.0655194 | 0.0933503 | 0.0597432 | | | |
| TG 49:1-FA15:0 | 0.989536 | 1.8586152 | 0.4895318 | 0.5562993 | 1.1085565 | 0.4088797 | 0.300707 | 0.3444821 | 0.5197119 |
| TG 49:1-FA16:0 | 1.3687916 | 2.701363 | 0.6487933 | 0.7714647 | 1.8497934 | 0.5934146 | 0.44824 | 0.5215411 | 0.6761129 |
| TG 49:1-FA16:1 | 0.1723497 | 0.3174026 | 0.1176754 | 0.1124368 | 0.1672165 | | 0.0987966 | 0.0965095 | 0.1029511 |
| TG 49:1-FA17:0 | 0.1266779 | 0.2328488 | 0.112335 | 0.0864908 | 0.1800349 | | | | |
| TG 49:1-FA18:1 | 1.250259 | 2.3768939 | 0.4224691 | 0.5680281 | 1.3318016 | 0.3729231 | 0.4003544 | 0.447332 | 0.5145094 |
| TG 49:2-FA14:0 | 0.1357355 | 0.2569424 | 0.1380895 | 0.133121 | 0.2333485 | 0.1064209 | | 0.0723419 | 0.0799252 |
| TG 49:2-FA15:0 | 2.1015253 | 3.9108146 | 0.8945485 | 1.3591402 | 2.4103255 | 0.6391225 | 0.5890924 | 0.8002866 | 0.9705412 |
| TG 49:2-FA16:0 | 2.1492294 | 4.1342668 | 0.7775787 | 1.4222534 | 2.6241571 | 0.7821387 | 0.677355 | 0.7913746 | 0.9627218 |
| TG 49:2-FA16:1 | 0.2330277 | 0.412197 | 0.1555394 | 0.1734692 | 0.5837931 | 0.0862098 | | 0.0999663 | 0.111738 |
| TG 49:2-FA17:0 | 0.1255553 | 0.1344032 | | 0.0910828 | 0.127281 | | | | |
| TG 49:2-FA18:1 | 0.3717967 | 0.5486727 | 0.2378246 | 0.2795934 | 0.5678498 | 0.1427549 | 0.0929981 | 0.124992 | 0.1989166 |
| TG 49:2-FA18:2 | 2.5505657 | 4.376715 | 0.6168179 | 1.5378502 | 2.5993322 | 0.6898973 | 0.776944 | 0.8223751 | 0.8898396 |
| TG 49:3-FA15:0 | 0.3816416 | 0.6303444 | 0.1570056 | 0.3010958 | 0.5301784 | | 0.0880526 | 0.1562116 | 0.1897655 |
| TG 49:3-FA16:0 | 0.4596661 | 0.5814927 | 0.2427742 | 0.3626458 | 0.2982254 | 0.2100325 | 0.1513887 | 0.1718328 | 0.1700299 |
| TG 49:3-FA16:1 | 0.312098 | 0.5310651 | 0.1753786 | 0.2894513 | 0.478486 | 0.1056665 | 0.0895974 | 0.1470993 | 0.1411855 |
| TG 49:3-FA18:2 | 0.6038418 | 0.8178718 | 0.2015184 | 0.4657836 | 0.6759775 | 0.1606131 | 0.1746377 | 0.2095839 | 0.244418 |
| TG 49:3-FA18:3 | 0.183188 | 0.3246273 | 0.0652334 | 0.13133 | 0.1524263 | | | 0.0685139 | 0.0543329 |
| TG 50:0-FA14:0 | | 0.1157022 | | | 0.0648073 | | | | |
| TG 50:0-FA16:0 | 1.7385367 | 4.2037095 | 1.2334604 | 1.023182 | 2.9272643 | 1.3540954 | 0.4953029 | 0.5971833 | 1.2372507 |
| TG 50:0-FA18:0 | 0.8952633 | 2.3917683 | 0.7721041 | 0.6122121 | 1.7408396 | 0.7612028 | 0.2798814 | 0.3849983 | 0.6119434 |
| TG 50:1-FA14:0 | 0.2132421 | 0.4620856 | 0.1428993 | 0.1069784 | 0.2484355 | 0.1223472 | 0.0550094 | 0.0945309 | 0.111029 |
| TG 50:1-FA16:0 | 20.206055 | 32.806106 | 12.624205 | 13.738692 | 47.559194 | 9.4640128 | 4.854393 | 9.5963082 | 10.790049 |
| TG 50:1-FA16:1 | 0.1661512 | 0.5638205 | 0.2682506 | 0.1793012 | 0.2782505 | 0.1085254 | 0.0657543 | 0.1407601 | 0.1647485 |
| TG 50:1-FA18:0 | 0.4426579 | 1.0055325 | 0.3352642 | 0.2186019 | 0.7266469 | 0.228732 | 0.135257 | 0.1710866 | 0.2008469 |
| TG 50:1-FA18:1 | 10.475148 | 17.141351 | 6.1954016 | 6.961298 | 23.788846 | 4.6625948 | 2.6620547 | 4.8351493 | 5.8782542 |
| TG 50:1-FA20:1 | 0.2131727 | 0.3757233 | 0.0804013 | 0.1118541 | 0.1175438 | 0.0852865 | | | |
| TG 50:2-FA14:0 | 1.2172966 | 2.7123375 | 0.8400772 | 1.2236028 | 6.42479 | 0.5991186 | 0.2398934 | 0.4930645 | 0.5797633 |
| TG 50:2-FA14:1 | 0.1303884 | 0.149473 | 0.1008407 | 0.10998 | 0.0433973 | | | | 0.0999695 |
| TG 50:2-FA16:0 | 51.014507 | 76.707291 | 28.266371 | 45.802092 | 107.17185 | 22.235409 | 13.746652 | 28.447751 | 26.279952 |
| TG 50:2-FA16:1 | 5.9961049 | 9.5039403 | 5.133641 | 5.0385675 | 26.781673 | 2.8437069 | 0.992189 | 3.1233334 | 4.0710917 |
| TG 50:2-FA18:0 | 0.3049174 | 0.730776 | 0.1772087 | 0.2065886 | 0.6180877 | 0.1710956 | 0.0942212 | 0.1448557 | 0.1496846 |
| TG 50:2-FA18:1 | 7.0665506 | 12.389412 | 6.0243903 | 6.1720382 | 35.415119 | 3.2052179 | 1.1576572 | 3.6213712 | 4.6669085 |
| TG 50:2-FA18:2 | 23.71183 | 35.450136 | 10.307431 | 20.696922 | 39.254841 | 9.3566721 | 6.2274702 | 11.902799 | 11.061987 |
| TG 50:2-FA20:2 | | | | | 0.06164 | | | | |
| TG 50:3-FA14:0 | 3.623682 | 5.7764403 | 2.3403926 | 5.257485 | 11.548497 | 1.2332112 | 0.7154104 | 1.5647695 | 1.6117622 |
| TG 50:3-FA14:1 | 0.2126432 | 0.2465451 | 0.166075 | 0.2186982 | 0.2586933 | 0.162391 | 0.0771609 | 0.0748739 | 0.1302865 |
| TG 50:3-FA16:0 | 14.719169 | 18.612614 | 10.081868 | 17.675096 | 35.853347 | 5.4036603 | 3.0962856 | 8.8005992 | 8.4620613 |
| TG 50:3-FA16:1 | 12.73435 | 16.450715 | 9.2212885 | 14.950269 | 36.872561 | 4.7890362 | 2.676799 | 7.1959101 | 7.7457749 |
| TG 50:3-FA18:0 | | 0.1131776 | 0.077899 | 0.073571 | 0.1181443 | | | | |
| TG 50:3-FA18:1 | 4.2126352 | 6.60748 | 2.9201248 | 5.8937859 | 14.947995 | 1.4942199 | 0.8464165 | 1.8974084 | 2.1132737 |
| TG 50:3-FA18:2 | 13.845912 | 18.027907 | 9.272558 | 17.197672 | 36.691674 | 4.726714 | 2.8172297 | 7.6350893 | 7.5313793 |
| TG 50:3-FA18:3 | 1.3018442 | 1.9548396 | 0.5221239 | 1.3354726 | 2.0031787 | 0.4490515 | 0.320034 | 0.6493627 | 0.5351026 |
| TG 50:3-FA20:3 | 0.0699712 | 0.1108695 | | 0.0704134 | 0.1105278 | | | | |
| TG 50:4-FA14:0 | 2.507966 | 3.0932905 | 1.3464545 | 4.9306791 | 4.9504436 | 0.7538583 | 0.6453794 | 1.2036131 | 0.9146527 |
| TG 50:4-FA14:1 | 0.2624027 | 0.2326347 | 0.2679361 | 0.4137757 | 0.2814395 | 0.1791654 | 0.1290964 | 0.1368892 | 0.2655566 |
| TG 50:4-FA16:0 | 1.4708135 | 1.4087594 | 0.743273 | 2.077307 | 1.9591848 | 0.4239849 | 0.3444892 | 0.6185209 | 0.6505588 |
| TG 50:4-FA16:1 | 1.4816865 | 1.8886702 | 1.0163863 | 1.8455621 | 5.2598366 | 0.3618434 | 0.2122738 | 0.5899408 | 0.7978607 |
| TG 50:4-FA18:1 | 0.3690337 | 0.4460105 | 0.2629019 | 0.5571159 | 0.7979076 | 0.1712636 | 0.0942686 | 0.174553 | 0.1909426 |
| TG 50:4-FA18:2 | 4.5425678 | 5.3339688 | 2.2806199 | 8.5478192 | 8.4227417 | 1.1895423 | 1.0901854 | 2.1403263 | 1.9109972 |
| TG 50:4-FA18:3 | 0.6606496 | 0.9907924 | 0.4597716 | 1.001795 | 1.8333224 | 0.1544803 | 0.1101395 | 0.3333869 | 0.3121793 |
| TG 50:4-FA20:3 | | | | | | | | | |
| TG 50:4-FA20:4 | 0.0646801 | 0.1261448 | | | 0.1985848 | | | | |
| TG 50:5-FA14:0 | 0.3223328 | 0.4364574 | 0.1652044 | 0.6843748 | 0.5653897 | 0.0780532 | 0.0860384 | 0.1329006 | 0.1193538 |
| TG 50:5-FA16:0 | 0.2571804 | 0.3584048 | 0.1539769 | 0.4001995 | 0.3470046 | 0.1701561 | 0.1093047 | 0.1671126 | 0.1626621 |
| TG 50:5-FA16:1 | 0.1817408 | 0.195928 | 0.1267102 | 0.2791307 | 0.4273918 | 0.0867258 | | 0.0664849 | 0.0844002 |
| TG 50:5-FA18:1 | 0.1560228 | 0.1656627 | 0.1202945 | 0.1982412 | 0.2090066 | 0.087091 | 0.0671443 | 0.0747955 | 0.1082261 |
| TG 50:5-FA18:2 | 0.4834991 | 0.5224745 | 0.3031421 | 0.9904097 | 0.6591747 | 0.1592286 | 0.1542758 | 0.1855344 | 0.2194404 |
| TG 50:5-FA18:3 | 0.3549089 | 0.4667227 | 0.2133222 | 0.8491628 | 0.7020479 | 0.0921028 | 0.0824123 | 0.1486907 | 0.1718694 |
| TG 50:5-FA20:4 | | | | | 0.0720192 | | | | |
| TG 50:5-FA20:5 | 0.0762016 | 0.1162824 | | 0.1309808 | 0.1205807 | | | | |
| TG 51:0-FA16:0 | 1.0147801 | 2.0203258 | 0.6167036 | 0.8232608 | 1.1532156 | 0.5438967 | 0.5148612 | 0.4024885 | 0.4786144 |
| TG 51:0-FA17:0 | 0.1364161 | 0.3671694 | 0.0874393 | 0.0709661 | 0.1458347 | 0.0911394 | 0.0623271 | 0.0555239 | 0.1452181 |
| TG 51:0-FA18:0 | 0.2197797 | 0.5421866 | 0.1618422 | 0.1937852 | 0.289462 | 0.1668634 | 0.1463227 | 0.1140164 | 0.2000654 |
| TG 51:1-FA15:0 | 0.4281813 | 1.0258313 | 0.1893054 | 0.2059613 | 0.2735381 | 0.1736771 | 0.1622362 | 0.1605917 | 0.2157684 |
| TG 51:1-FA16:0 | 1.8936352 | 3.7927685 | 1.0097799 | 1.228983 | 2.4305617 | 0.9312853 | 0.5728276 | 0.7071291 | 1.0107052 |
| TG 51:1-FA17:0 | 1.137013 | 2.2571399 | 0.6843432 | 0.7380632 | 1.544197 | 0.6193492 | 0.2917443 | 0.4475011 | 0.6123094 |
| TG 51:1-FA18:0 | 0.3552161 | 0.827626 | 0.1869367 | 0.2257725 | 0.2900804 | 0.1732907 | 0.178494 | 0.1457966 | 0.2001548 |
| TG 51:1-FA18:1 | 1.3835332 | 2.8066154 | 0.5820797 | 0.9003731 | 1.7015496 | 0.5403002 | 0.5322165 | 0.4021909 | 0.5359637 |
| TG 51:2-FA15:0 | 1.2621304 | 2.2112927 | 0.4706515 | 0.8758101 | 2.174223 | 0.3931735 | 0.3431045 | 0.4752969 | 0.5493983 |
| TG 51:2-FA16:0 | 4.8165395 | 7.4225009 | 2.8506277 | 3.3984852 | 6.7275964 | 2.2361548 | 1.2539938 | 1.9430639 | 2.878816 |
| TG 51:2-FA16:1 | 0.3058574 | 0.5537085 | 0.2843025 | 0.2028671 | 0.6015535 | 0.1746657 | 0.0805017 | 0.1515742 | 0.2370841 |
| TG 51:2-FA17:0 | 2.353148 | 4.3125352 | 1.170569 | 2.0832028 | 3.2444468 | 1.098569 | 0.7219494 | 1.0092894 | 1.2391149 |
| TG 51:2-FA18:1 | 3.0857243 | 4.9010568 | 1.3589471 | 2.2085953 | 6.5262532 | 1.0361716 | 0.8522748 | 1.0976505 | 1.5048024 |
| TG 51:2-FA18:2 | 3.1321982 | 5.1468995 | 0.8307908 | 2.159527 | 3.5181262 | 0.9368534 | 1.0398585 | 1.0405638 | 1.0774503 |
| TG 51:3-FA15:0 | 3.1564492 | 4.344325 | 0.9305341 | 2.8955495 | 4.2259718 | 0.7711559 | 0.6658132 | 1.0814273 | 1.1893901 |
| TG 51:3-FA16:1 | 0.7591537 | 1.0249801 | 0.5824078 | 0.541174 | 1.2394667 | 0.3074452 | 0.143291 | 0.2157391 | 0.5180954 |
| TG 51:3-FA17:0 | 0.406029 | 0.6722363 | 0.2061497 | 0.3794889 | 0.6115143 | 0.1469259 | | 0.1305047 | 0.234505 |
| TG 51:3-FA18:2 | 4.8184627 | 6.5154981 | 1.6327827 | 4.2720053 | 6.6525882 | 1.2256326 | 1.1127957 | 1.6562258 | 1.8284447 |
| TG 51:3-FA18:3 | 0.186385 | 0.3792968 | 0.0751343 | 0.1229186 | 0.1814675 | 0.0538236 | 0.0820977 | 0.0586404 | 0.105916 |
| TG 51:4-FA15:0 | 3.1605252 | 3.6937731 | 0.6929097 | 3.8073603 | 2.8370631 | 0.55594 | 0.7233956 | 1.0663561 | 1.0541799 |
| TG 51:4-FA16:1 | 0.3209095 | 0.3738898 | 0.198145 | 0.3402467 | 0.5543998 | 0.1168737 | 0.0533625 | 0.1198153 | 0.1769904 |
| TG 51:4-FA18:2 | 5.7316934 | 6.7045284 | 1.3817138 | 6.4989142 | 5.248458 | 1.1092437 | 1.4094339 | 1.9270331 | 1.9091655 |
| TG 51:4-FA18:3 | 0.4129971 | 0.5638143 | 0.1324107 | 0.4648169 | 0.4958972 | 0.1033318 | 0.1054104 | 0.1202322 | 0.1449943 |
| TG 51:4-FA20:4 | 0.1231599 | 0.2965333 | | 0.1146094 | 0.2453456 | | | | 0.0800788 |
| TG 51:5-FA18:2 | 0.5757298 | 0.6025553 | 0.1605176 | 0.6989972 | 0.4133037 | 0.1294699 | 0.1590393 | 0.1889852 | 0.1800415 |
| TG 51:5-FA18:3 | 0.4005831 | 0.5187775 | | 0.5969538 | 0.4065282 | 0.0797761 | 0.0789669 | 0.1424043 | 0.113302 |
| TG 52:0-FA16:0 | 0.7667196 | 1.8660358 | 0.4970621 | 0.6089555 | 1.0093248 | 0.4829108 | 0.2772335 | 0.3613799 | 0.4128438 |
| TG 52:0-FA18:0 | 0.5552973 | 1.2997236 | 0.5615337 | 0.3683941 | 1.0564746 | 0.5374302 | 0.198423 | 0.2986512 | 0.5714714 |
| TG 52:0-FA20:0 | 1.5642292 | 2.7501728 | 1.0154325 | 1.1054936 | 1.8984971 | 0.8362214 | 0.4311252 | 0.682129 | 0.974878 |
| TG 52:1-FA16:0 | 6.6736361 | 12.911136 | 3.2718375 | 3.7852997 | 9.0933697 | 2.7545041 | 1.4604231 | 2.2041307 | 2.7537787 |
| TG 52:1-FA16:1 | 0.1549066 | 0.2577941 | 0.0831889 | 0.1135259 | 0.1148525 | 0.0671226 | 0.0361242 | 0.0607646 | 0.0864084 |
| TG 52:1-FA18:0 | 3.9345866 | 8.1756393 | 2.1515479 | 2.5624653 | 7.7301599 | 1.9965096 | 1.2917519 | 1.7120894 | 1.7012358 |
| TG 52:1-FA18:1 | 3.388741 | 6.7729238 | 1.8663996 | 2.2937059 | 7.8622235 | 1.6569039 | 1.2384972 | 1.6358227 | 1.6538934 |
| TG 52:1-FA20:0 | 0.7973263 | 1.3744222 | 0.7153314 | 0.7888438 | 1.4533446 | 0.3864153 | 0.1690192 | 0.4418918 | 0.5898947 |
| TG 52:1-FA20:1 | 2.3136221 | 4.1605576 | 0.8004408 | 1.0860512 | 1.7306041 | 0.7130532 | 0.3428215 | 0.4859449 | 0.6624744 |
| TG 52:2-FA14:0 | 0.23131 | 0.4331976 | 0.1362914 | 0.1752187 | 0.2115223 | 0.1176631 | | 0.0838903 | 0.1394508 |
| TG 52:2-FA16:0 | 51.347742 | 72.601579 | 32.698586 | 41.531911 | 156.48854 | 25.763981 | 11.036686 | 24.918562 | 33.087643 |
| TG 52:2-FA16:1 | 1.04801 | 1.7831668 | 0.6319507 | 0.6070308 | 1.6604339 | 0.3954152 | 0.1861579 | 0.379815 | 0.4524465 |
| TG 52:2-FA18:0 | 8.8777699 | 18.15017 | 4.3728922 | 7.1789971 | 15.421554 | 4.291041 | 3.3170591 | 4.3863876 | 4.1429678 |
| TG 52:2-FA18:1 | 78.840597 | 105.45099 | 52.709204 | 62.862329 | 253.78758 | 39.265478 | 14.277924 | 35.306177 | 52.050357 |
| TG 52:2-FA18:2 | 7.2875589 | 15.50435 | 3.3030745 | 6.0993638 | 12.108616 | 3.2718052 | 2.9500625 | 3.6872417 | 3.3148391 |
| TG 52:2-FA20:0 | 0.4149579 | 0.5146112 | 0.2873244 | 0.5373785 | 0.4940052 | 0.1783756 | 0.1241431 | 0.1943127 | 0.1878847 |
| TG 52:2-FA20:1 | 0.976173 | 1.4958016 | 0.522372 | 0.4680225 | 0.7762603 | 0.3593247 | 0.1385536 | 0.2575165 | 0.3360672 |
| TG 52:2-FA20:2 | 0.2128131 | 0.3757847 | 0.1302183 | 0.1724958 | 0.3733905 | 0.1431585 | 0.0767898 | 0.1343119 | 0.1099008 |
| TG 52:3-FA14:0 | 0.353947 | 0.6282222 | 0.185375 | 0.2573695 | 0.3666779 | 0.1301628 | 0.0685963 | 0.0787219 | 0.1533257 |
| TG 52:3-FA16:0 | 181.04128 | 180.14552 | 95.709051 | 196.77608 | 323.61873 | 65.853039 | 42.294144 | 89.2412 | 99.862297 |
| TG 52:3-FA16:1 | 4.8456081 | 7.4150673 | 3.4103961 | 4.3492524 | 23.221432 | 2.0021662 | 0.9314868 | 1.706596 | 3.3652718 |
| TG 52:3-FA18:0 | 1.3141802 | 2.4687484 | 0.8346093 | 1.2972385 | 3.1247535 | 0.6062168 | 0.4008462 | 0.6970274 | 0.8490305 |
| TG 52:3-FA18:1 | 169.58836 | 174.78669 | 90.370866 | 182.09429 | 322.53208 | 61.303354 | 38.844853 | 83.368946 | 93.812569 |
| TG 52:3-FA18:2 | 155.66159 | 157.90813 | 78.432066 | 164.18036 | 269.5879 | 55.053324 | 34.817633 | 74.977729 | 83.461188 |
| TG 52:3-FA18:3 | 0.4461283 | 0.7340743 | 0.191644 | 0.3321944 | 0.5919066 | 0.1554355 | 0.1240236 | 0.1951167 | 0.2011448 |
| TG 52:3-FA20:0 | 0.4609591 | 0.3994232 | 0.2261416 | 0.5026327 | 0.5923641 | 0.1598806 | 0.0953997 | 0.2047656 | 0.268778 |
| TG 52:3-FA20:1 | 0.5327219 | 0.8965884 | 0.286492 | 0.5070502 | 0.4956281 | 0.150281 | 0.0951005 | 0.1677819 | 0.1960536 |
| TG 52:3-FA20:2 | 0.3439007 | 0.4708301 | 0.1606374 | 0.2791688 | 0.7843106 | 0.112975 | 0.0709968 | 0.142475 | 0.1254268 |
| TG 52:3-FA20:3 | 0.2892428 | 0.5333645 | 0.1260413 | 0.2164418 | 0.8098528 | 0.1204244 | 0.1152723 | 0.1449751 | 0.167794 |
| TG 52:3-FA22:1 | 0.0987505 | 0.0727133 | | 0.1083324 | 0.2058698 | | | 0.0714696 | 0.0734819 |
| TG 52:4-FA14:0 | 0.1573287 | 0.2790948 | 0.1010939 | 0.1864421 | 0.4482268 | 0.063036 | 0.0705714 | 0.0766724 | |
| TG 52:4-FA16:0 | 185.92391 | 142.83765 | 69.920523 | 257.26482 | 187.13792 | 47.397475 | 48.273845 | 89.631471 | 77.960762 |
| TG 52:4-FA16:1 | 14.264977 | 16.070153 | 7.6150828 | 17.092676 | 40.883483 | 3.7306677 | 2.2217436 | 4.7611071 | 7.7469231 |
| TG 52:4-FA18:0 | 0.2580136 | 0.3936901 | 0.1255007 | 0.2344724 | 0.3148492 | 0.1457625 | 0.1098372 | 0.1285969 | 0.1111383 |
| TG 52:4-FA18:1 | 24.106637 | 24.206976 | 11.852289 | 30.81756 | 52.179465 | 6.3919623 | 4.5055092 | 9.564145 | 11.551861 |
| TG 52:4-FA18:2 | 295.97564 | 231.66394 | 111.6886 | 403.78464 | 307.82381 | 74.914788 | 76.726134 | 141.66353 | 121.63261 |
| TG 52:4-FA18:3 | 16.038892 | 15.533379 | 6.9577627 | 21.128513 | 24.344823 | 4.1830855 | 3.7935009 | 7.4287721 | 6.9313628 |
| TG 52:4-FA20:0 | 1.1737376 | 0.9894146 | 0.4644823 | 1.7258333 | 1.184534 | 0.3530299 | 0.3749383 | 0.5964959 | 0.5841467 |
| TG 52:4-FA20:2 | 0.2617765 | 0.3338533 | 0.1373847 | 0.3116947 | 0.3948818 | 0.1044135 | 0.08033 | 0.1096558 | 0.1149723 |
| TG 52:4-FA20:3 | 0.4842615 | 0.636611 | 0.2183272 | 0.5335679 | 1.3507033 | 0.1437358 | 0.1212525 | 0.2360233 | 0.1755522 |
| TG 52:4-FA20:4 | 0.9703097 | 1.880009 | 0.4297368 | 0.8431304 | 3.2612035 | 0.3705178 | 0.3952276 | 0.6016983 | 0.4521094 |
| TG 52:4-FA22:1 | 0.2526431 | 0.1988609 | 0.1116071 | 0.3338102 | 0.3180693 | 0.0672598 | 0.0865242 | 0.1211843 | 0.1334744 |
| TG 52:4-FA22:4 | 0.1501178 | 0.0961093 | | 0.1289064 | 0.1405898 | | | | |
| TG 52:5-FA14:0 | 0.1423183 | 0.2945493 | 0.0771618 | 0.1742816 | 0.4657855 | | 0.0671772 | 0.0727901 | 0.067626 |
| TG 52:5-FA16:0 | 17.281363 | 14.385162 | 6.9020845 | 32.478984 | 19.03565 | 3.7027739 | 4.2187874 | 8.335151 | 6.3216119 |
| TG 52:5-FA16:1 | 12.812018 | 10.794593 | 5.1566616 | 20.165798 | 19.407056 | 2.4168817 | 2.2503708 | 4.1506541 | 5.4321902 |
| TG 52:5-FA18:1 | 2.3129102 | 2.1837065 | 1.020457 | 3.6314217 | 4.0795454 | 0.4955626 | 0.4642747 | 0.8350168 | 1.0427918 |
| TG 52:5-FA18:2 | 30.945092 | 25.564074 | 11.890553 | 50.901901 | 39.746181 | 6.0951991 | 5.9580736 | 11.94356 | 12.246739 |
| TG 52:5-FA18:3 | 21.349343 | 16.945245 | 8.0589736 | 35.975041 | 23.009627 | 3.8960544 | 4.8429374 | 9.4114437 | 7.7788574 |
| TG 52:5-FA20:3 | 0.1645813 | 0.2836253 | 0.0713106 | 0.2225641 | 0.4590222 | 0.0698689 | | 0.0777417 | |
| TG 52:5-FA20:4 | 0.4674603 | 0.9588979 | 0.2895049 | 0.5087024 | 1.8919094 | 0.1752022 | 0.1622974 | 0.3057629 | 0.2017303 |
| TG 52:5-FA20:5 | 0.8289374 | 1.3730434 | 0.441899 | 1.3083937 | 1.2697554 | 0.4099726 | 0.4315259 | 0.6623686 | 0.5309 |
| TG 52:5-FA22:5 | 0.4219201 | 0.3734419 | 0.1978693 | 0.7888478 | 0.5648389 | 0.1358156 | 0.1337204 | 0.1797918 | 0.149192 |
| TG 52:6-FA14:0 | 0.1963823 | 0.4007413 | 0.1853534 | 0.3697224 | 0.6343868 | 0.1660321 | 0.1638423 | 0.1345684 | 0.1553651 |
| TG 52:6-FA16:0 | 1.3458147 | 1.2868228 | 0.6678608 | 3.2633647 | 1.6720362 | 0.2917484 | 0.4675045 | 0.8310068 | 0.6163659 |
| TG 52:6-FA16:1 | 1.6973041 | 1.4726429 | 0.7086077 | 3.282791 | 2.8444645 | 0.3288712 | 0.3489234 | 0.6109406 | 0.6089058 |
| TG 52:6-FA18:1 | 0.3379649 | 0.3225115 | 0.1379403 | 0.546765 | 0.518861 | 0.1229481 | 0.1122953 | 0.1499241 | 0.156928 |
| TG 52:6-FA18:2 | 3.1684179 | 2.3620931 | 1.1252429 | 6.3317111 | 3.4404867 | 0.4725528 | 0.7327391 | 1.1397945 | 1.1487604 |
| TG 52:6-FA18:3 | 2.4109433 | 2.1079644 | 0.9890983 | 4.8304662 | 3.4678901 | 0.3911332 | 0.3610599 | 0.8531638 | 0.9525923 |
| TG 52:6-FA20:4 | 0.2458125 | 0.4905146 | 0.1156385 | 0.3828444 | 0.7881337 | 0.0812222 | 0.1182392 | 0.1494284 | 0.1218505 |
| TG 52:6-FA20:5 | 0.557853 | 0.915862 | 0.4702864 | 1.2261468 | 1.4415624 | 0.2647841 | 0.2227082 | 0.4679251 | 0.3611298 |
| TG 52:6-FA22:6 | 0.4731978 | 1.1556833 | 0.3175255 | 0.7571761 | 1.483197 | 0.2979659 | 0.2412914 | 0.3627604 | 0.2225201 |
| TG 52:7-FA16:0 | 0.8033576 | 1.7231856 | 0.5617685 | 0.6555697 | 0.9242378 | 0.454794 | 0.4134891 | 0.2986108 | 0.3810906 |
| TG 52:7-FA18:1 | 0.0416918 | 0.1035322 | 0.0676006 | 0.1278802 | 0.0913156 | 0.0334788 | 0.0316713 | 0.0651681 | 0.0663145 |
| TG 52:7-FA20:5 | 0.3435659 | 0.4885623 | 0.2344919 | 1.2394879 | 0.6835077 | 0.1511984 | 0.1622765 | 0.2704014 | 0.1804205 |
| TG 52:7-FA22:6 | 0.2296282 | 0.454363 | 0.1574212 | 0.3266334 | 0.6999448 | 0.193198 | 0.0970626 | 0.1466356 | 0.1056926 |
| TG 52:8-FA16:1 | 0.1197606 | 0.180051 | 0.0892686 | 0.1118095 | 0.1157884 | | | | 0.0701193 |
| TG 52:8-FA18:2 | 0.061652 | 0.0964738 | 0.0451666 | 0.1801717 | 0.081518 | 0.0401817 | 0.0851179 | 0.0444989 | 0.0894594 |
| TG 53:0-FA16:0 | 4.2476129 | 5.1425623 | 2.1415798 | 11.351471 | 5.9499175 | 1.827885 | 2.3442901 | 3.3086784 | 2.3822572 |
| TG 53:1-FA16:0 | 1.5009644 | 2.909695 | 0.8206321 | 1.4252911 | 1.5266434 | 0.6893881 | 0.7095471 | 0.487203 | 0.646575 |
| TG 53:1-FA17:0 | 0.3703054 | 0.800083 | 0.1756088 | 0.2485067 | 0.3187249 | 0.1705199 | 0.1276866 | 0.1615272 | 0.204384 |
| TG 53:1-FA18:0 | 0.5438557 | 1.1340122 | 0.2694021 | 0.3844799 | 0.5001003 | 0.2783192 | 0.2921021 | 0.2387557 | 0.3247772 |
| TG 53:1-FA18:1 | 0.9672801 | 1.9274277 | 0.4139237 | 0.7468737 | 0.9686957 | 0.3124664 | 0.4938602 | 0.2628047 | 0.3173841 |
| TG 53:2-FA16:0 | 3.8677874 | 5.0276666 | 2.8066304 | 2.4469059 | 4.3340831 | 2.4028008 | 0.8405893 | 1.1701859 | 2.6587353 |
| TG 53:2-FA17:0 | 1.0904251 | 2.1222605 | 0.6374168 | 0.8178988 | 2.2898647 | 0.4702452 | 0.3789151 | 0.3956845 | 0.6570506 |
| TG 53:2-FA18:1 | 4.676915 | 6.5228307 | 3.0495456 | 3.1563786 | 6.5363422 | 2.3230368 | 1.1633427 | 1.5735761 | 2.7798894 |
| TG 53:2-FA18:2 | 2.027812 | 3.3584349 | 0.6606684 | 1.8593276 | 2.2269892 | 0.7000443 | 0.8472252 | 0.6284516 | 0.7055888 |
| TG 53:3-FA16:0 | 11.580927 | 9.7332919 | 8.126143 | 7.7522358 | 7.4183439 | 5.608197 | 1.8544451 | 3.0499146 | 7.9576612 |
| TG 53:3-FA17:0 | 2.9251069 | 4.4721229 | 1.3177721 | 2.6603213 | 4.8343503 | 1.0032557 | 0.7077917 | 1.2678076 | 1.4621251 |
| TG 53:3-FA18:2 | 6.6950003 | 8.1320557 | 3.0699424 | 5.0601934 | 6.7959132 | 2.3382821 | 1.6476802 | 2.0911642 | 3.0441914 |
| TG 53:4-FA16:0 | 14.015095 | 8.7773144 | 7.8768571 | 10.706518 | 4.8061391 | 4.8518635 | 2.1171963 | 3.20041 | 7.5043792 |
| TG 53:4-FA17:0 | 2.8977778 | 3.7951498 | 0.9096981 | 3.2196128 | 3.2253547 | 0.7900637 | 0.9104404 | 1.2134321 | 1.2229359 |
| TG 53:4-FA18:2 | 14.918321 | 13.766074 | 6.869217 | 13.539777 | 10.637015 | 4.977886 | 3.1627442 | 4.2776544 | 7.134322 |
| TG 53:4-FA18:3 | 0.6388243 | 0.7468301 | 0.2755655 | 0.6091159 | 0.6059673 | 0.1858023 | 0.1842375 | 0.198834 | 0.2763517 |
| TG 53:4-FA20:4 | 0.1272693 | 0.3075362 | | 0.1111762 | 0.3107857 | 0.0720811 | | 0.1023619 | 0.1187515 |
| TG 53:5-FA20:4 | 0.1424439 | 0.335604 | | 0.1366011 | 0.3803494 | | 0.0793691 | 0.1026881 | 0.1100391 |
| TG 53:6-FA20:4 | 0.2003996 | 0.3525955 | 0.0681078 | 0.278286 | 0.3769433 | 0.0917831 | 0.0839878 | 0.0988559 | 0.0952365 |
| TG 54:0-FA16:0 | 1.0569358 | 1.5632636 | 0.5856054 | 1.7042069 | 1.3154956 | 0.4472048 | 0.3518319 | 0.5610114 | 0.5156977 |
| TG 54:0-FA18:0 | 0.204024 | 0.5755823 | 0.1462488 | 0.1497092 | 0.1786609 | 0.1883626 | 0.1113161 | 0.1572485 | 0.1544483 |
| TG 54:1-FA16:0 | 1.4279457 | 3.0577171 | 0.4893788 | 0.9606405 | 1.0713997 | 0.5899687 | 0.3996565 | 0.4917901 | 0.4085917 |
| TG 54:1-FA18:0 | 1.1947155 | 2.4174192 | 0.6927467 | 0.5998305 | 1.3223864 | 0.7293486 | 0.3538761 | 0.4091904 | 0.6501827 |
| TG 54:1-FA18:1 | 1.11571 | 2.1308895 | 0.4470423 | 0.7224298 | 1.2188366 | 0.4564969 | 0.4016515 | 0.4283797 | 0.414358 |
| TG 54:1-FA20:0 | 8.5050865 | 10.121475 | 6.3978143 | 7.1241236 | 10.087558 | 4.2284494 | 1.9543683 | 3.7736812 | 5.8397347 |
| TG 54:1-FA20:1 | 0.6691468 | 1.4731525 | 0.3438378 | 0.3968918 | 0.5297647 | 0.2844958 | 0.195814 | 0.1940972 | 0.283856 |
| TG 54:2-FA16:0 | 8.9526236 | 11.771103 | 5.1812491 | 4.5608536 | 5.7873675 | 3.8451957 | 1.5200884 | 2.1897501 | 4.2476905 |
| TG 54:2-FA18:0 | 3.2069326 | 6.1000954 | 1.7009314 | 2.3309177 | 7.9013534 | 1.7052811 | 1.1611859 | 1.3806223 | 1.6555262 |
| TG 54:2-FA18:1 | 10.091905 | 13.593951 | 5.4094737 | 5.2547266 | 13.065187 | 4.0934337 | 1.9170657 | 2.2060795 | 4.4030024 |
| TG 54:2-FA18:2 | 2.0825833 | 4.4412165 | 0.5619782 | 1.7081427 | 1.833802 | 0.6957843 | 0.7368398 | 0.7157369 | 0.4633475 |
| TG 54:2-FA20:0 | 13.894576 | 14.092234 | 7.4701326 | 14.970468 | 10.497237 | 5.0828516 | 3.7198724 | 5.752682 | 7.3380866 |
| TG 54:2-FA20:1 | 7.9622434 | 10.546174 | 3.9009402 | 3.8762914 | 6.2155318 | 2.9347702 | 1.2237901 | 1.9495844 | 3.2305137 |
| TG 54:2-FA20:2 | 0.1020482 | 0.1422435 | 0.0542521 | 0.0496907 | 0.1049736 | 0.0491535 | 0.0470432 | 0.060971 | 0.0422958 |
| TG 54:3-FA16:0 | 16.014394 | 18.164054 | 7.3646433 | 9.9761803 | 11.515439 | 5.8636621 | 2.8155022 | 3.7949985 | 6.4413979 |
| TG 54:3-FA16:1 | 0.8785092 | 1.3130753 | 0.4638668 | 0.4821963 | 0.9017489 | 0.3206534 | 0.1387181 | 0.259015 | 0.4233693 |
| TG 54:3-FA18:0 | 11.736116 | 18.745361 | 4.8054504 | 9.681852 | 21.786265 | 4.4475018 | 4.294082 | 4.099525 | 5.3136594 |
| TG 54:3-FA18:1 | 28.354089 | 37.656194 | 14.751021 | 22.466815 | 97.473811 | 12.149628 | 7.1306348 | 8.8347273 | 14.931585 |
| TG 54:3-FA18:2 | 21.296271 | 27.450957 | 8.1920195 | 14.290893 | 22.681314 | 7.0078536 | 5.1124369 | 5.5779731 | 7.907672 |
| TG 54:3-FA18:3 | 0.1276077 | 0.2167008 | 0.0331631 | 0.1086509 | 0.050051 | 0.0380713 | 0.0387601 | 0.0510111 | 0.0742547 |
| TG 54:3-FA20:1 | 15.063265 | 18.275388 | 5.8672089 | 10.252127 | 10.30758 | 4.722779 | 2.6443797 | 3.4605367 | 5.2440971 |
| TG 54:3-FA20:2 | 1.7010212 | 2.5510648 | 0.693238 | 1.2166526 | 3.9090036 | 0.8761056 | 0.377318 | 0.6594668 | 0.6102231 |
| TG 54:3-FA20:3 | 0.1098026 | 0.218013 | 0.0758284 | 0.1118528 | 0.2582083 | 0.1002751 | 0.0675749 | 0.0517904 | 0.0793337 |
| TG 54:4-FA16:0 | 5.067734 | 6.8801603 | 2.0502311 | 4.4960171 | 10.018572 | 2.1214478 | 1.3467354 | 2.0334153 | 1.7303775 |
| TG 54:4-FA16:1 | 1.3967218 | 1.9647968 | 0.6091993 | 1.0409467 | 1.5486684 | 0.3522361 | 0.1980524 | 0.3229507 | 0.4952425 |
| TG 54:4-FA18:0 | 14.611005 | 21.996436 | 4.2657841 | 14.794077 | 21.806684 | 5.0536298 | 5.0809828 | 5.3803371 | 5.9356057 |
| TG 54:4-FA18:1 | 60.789947 | 58.575622 | 26.589805 | 59.242975 | 154.05339 | 19.179928 | 12.354518 | 17.505698 | 28.617981 |
| TG 54:4-FA18:2 | 49.524948 | 60.41503 | 18.23993 | 48.70205 | 99.605313 | 16.201881 | 13.324545 | 16.076401 | 21.640582 |
| TG 54:4-FA18:3 | 1.5515854 | 2.3160286 | 0.6248832 | 1.5111608 | 1.9645666 | 0.554016 | 0.3993388 | 0.4842895 | 0.564307 |
| TG 54:4-FA20:1 | 1.7223785 | 2.3979807 | 0.6522999 | 1.3500837 | 1.4302019 | 0.4167508 | 0.2539765 | 0.4100528 | 0.5611465 |
| TG 54:4-FA20:2 | 2.7935661 | 3.8854658 | 1.0480813 | 3.0335924 | 4.8757397 | 1.2333806 | 0.7824843 | 1.2062879 | 0.8719666 |
| TG 54:4-FA20:3 | 2.3682542 | 3.7875632 | 0.9465084 | 1.8252945 | 7.4214488 | 0.9960642 | 0.5788144 | 0.8315327 | 0.7844698 |
| TG 54:4-FA20:4 | 0.6648643 | 1.4413825 | 0.3442315 | 0.6271232 | 1.8090936 | 0.304051 | 0.363295 | 0.4099971 | 0.4470534 |
| TG 54:4-FA22:1 | 0.1391211 | 0.1500239 | 0.0980523 | 0.1580665 | 0.2986907 | | | 0.0626116 | 0.0701874 |
| TG 54:4-FA22:4 | 0.0839699 | 0.1598012 | | 0.0959767 | 0.2108604 | 0.0595553 | | 0.0567502 | |
| TG 54:5-FA16:0 | 5.4849618 | 7.2358832 | 1.9172768 | 5.6786241 | 11.712665 | 1.881555 | 1.7428546 | 2.6757354 | 1.9395094 |
| TG 54:5-FA16:1 | 0.5268087 | 0.8066427 | 0.2941134 | 0.4988614 | 1.8003871 | 0.1909366 | 0.1275973 | 0.1620395 | 0.2330602 |
| TG 54:5-FA18:0 | 1.3585911 | 1.9107719 | 0.4378629 | 1.5489353 | 1.8060995 | 0.4298074 | 0.5093921 | 0.5418039 | 0.50865 |
| TG 54:5-FA18:1 | 50.495387 | 42.947529 | 16.398558 | 62.290234 | 82.049093 | 12.617612 | 11.53721 | 14.98279 | 19.211858 |
| TG 54:5-FA18:2 | 80.086869 | 65.378237 | 24.367219 | 94.890687 | 117.91932 | 18.995343 | 18.463668 | 23.550998 | 27.864324 |
| TG 54:5-FA18:3 | 5.3209883 | 5.6346057 | 2.0585598 | 6.4244574 | 10.494697 | 1.4148556 | 1.2242321 | 1.7489764 | 2.1359098 |
| TG 54:5-FA20:2 | 0.4414614 | 0.6089485 | 0.1965479 | 0.5306645 | 1.0449836 | 0.1626243 | 0.1271871 | 0.1525872 | 0.1439411 |
| TG 54:5-FA20:3 | 4.8872562 | 5.6034911 | 1.670362 | 4.7805456 | 9.4471424 | 1.5783771 | 1.3714596 | 1.8364554 | 1.4916157 |
| TG 54:5-FA20:4 | 5.5003029 | 8.4962377 | 2.2312399 | 4.5192968 | 15.135327 | 2.0042557 | 2.0442396 | 3.2588527 | 2.1185717 |
| TG 54:5-FA20:5 | 0.2553351 | 0.5361597 | 0.1817917 | 0.3447166 | 0.3614581 | 0.1068589 | 0.1816636 | 0.1700497 | 0.2108606 |
| TG 54:5-FA22:1 | 0.303228 | 0.3117231 | 0.1183782 | 0.4557591 | 0.5075514 | 0.1288762 | 0.0745147 | 0.1087124 | 0.1477422 |
| TG 54:5-FA22:4 | 0.127272 | 0.2235992 | | 0.0866534 | 0.3875151 | 0.0671353 | | 0.0653657 | |
| TG 54:5-FA22:5 | 0.4660031 | 0.9325174 | 0.1970533 | 0.5637416 | 0.9939637 | 0.2950298 | 0.2029493 | 0.3710758 | 0.240676 |
| TG 54:6-FA16:0 | 6.0335258 | 8.9822801 | 2.6365311 | 8.5308395 | 12.560496 | 2.5825391 | 2.9072743 | 4.2750503 | 2.844582 |
| TG 54:6-FA16:1 | 0.599089 | 0.9293616 | 0.2740962 | 0.7882746 | 1.9378389 | 0.214861 | 0.1693474 | 0.2913428 | 0.2216829 |
| TG 54:6-FA18:1 | 10.326884 | 8.7227667 | 3.7709438 | 17.379091 | 15.198016 | 2.4781498 | 2.4147458 | 3.7222572 | 4.0015923 |
| TG 54:6-FA18:2 | 61.815759 | 51.262882 | 14.954941 | 92.054533 | 74.240611 | 12.625314 | 16.212122 | 19.255297 | 18.095346 |
| TG 54:6-FA18:3 | 9.6676737 | 7.590768 | 3.3041114 | 16.468592 | 13.546561 | 2.1316227 | 2.0472388 | 3.3053318 | 3.4731654 |
| TG 54:6-FA20:3 | 0.5998054 | 0.7665343 | 0.2762541 | 0.7344715 | 1.5857257 | 0.1893737 | 0.1600041 | 0.2399598 | 0.1815231 |
| TG 54:6-FA20:4 | 10.949542 | 15.132435 | 3.9644147 | 12.588459 | 22.148227 | 3.3973996 | 5.2627358 | 7.774596 | 3.9128609 |
| TG 54:6-FA20:5 | 5.9378846 | 7.555447 | 3.5489331 | 11.826296 | 10.364955 | 2.9214052 | 2.7336194 | 4.6699597 | 3.934164 |
| TG 54:6-FA22:5 | 0.5148296 | 0.9914072 | 0.3051514 | 0.7401607 | 1.7829821 | 0.3111672 | 0.1901904 | 0.4046401 | 0.2141926 |
| TG 54:6-FA22:6 | 4.1298577 | 8.0659232 | 2.1072532 | 5.1986955 | 10.837531 | 2.6602073 | 2.2491477 | 3.4703786 | 2.3692195 |
| TG 54:7-FA16:1 | 0.812307 | 1.2961233 | 0.4478898 | 1.224008 | 2.495599 | 0.3902063 | 0.3227032 | 0.507817 | 0.4461705 |
| TG 54:7-FA18:1 | 1.218722 | 1.2654578 | 0.4700027 | 2.2553695 | 2.0779531 | 0.2601168 | 0.2732722 | 0.4515718 | 0.5387502 |
| TG 54:7-FA18:2 | 13.580665 | 9.9183941 | 4.1356057 | 32.664689 | 12.098132 | 2.5521404 | 3.8994476 | 5.7322653 | 4.5318738 |
| TG 54:7-FA18:3 | 7.4319229 | 4.8019905 | 1.999982 | 16.187294 | 6.4370905 | 1.102961 | 1.6560765 | 2.3634659 | 2.1995242 |
| TG 54:7-FA20:4 | 1.2437929 | 1.5154882 | 0.528879 | 1.7455687 | 2.4845769 | 0.3321628 | 0.5169868 | 0.6340835 | 0.4284314 |
| TG 54:7-FA20:5 | 14.678852 | 14.33 | 7.0986064 | 42.107727 | 15.643035 | 5.694712 | 8.3335617 | 11.540528 | 8.2479426 |
| TG 54:7-FA22:5 | 0.2979901 | 0.5651939 | 0.2502103 | 0.8693312 | 0.75681 | 0.1812628 | 0.168748 | 0.2236787 | 0.1451852 |
| TG 54:7-FA22:6 | 3.3662908 | 8.1844578 | 1.9329091 | 4.5108846 | 14.635593 | 1.9559613 | 1.1127317 | 2.1270857 | 1.4586173 |
| TG 54:8-FA18:2 | 1.5621241 | 1.618388 | 0.5938083 | 4.1295588 | 2.025519 | 0.4122099 | 0.474086 | 0.691645 | 0.5334023 |
| TG 54:8-FA18:3 | 1.3265494 | 0.9326624 | 0.3806759 | 3.7219076 | 1.1262548 | 0.2252398 | 0.2900356 | 0.5076647 | 0.4219699 |
| TG 54:8-FA20:4 | 0.1603936 | 0.1507687 | | 0.2531671 | 0.211523 | | 0.0848964 | 0.0868917 | 0.0689913 |
| TG 54:8-FA20:5 | 1.5649698 | 1.6704649 | 0.8519091 | 4.9086282 | 1.9625409 | 0.4978126 | 0.7103545 | 0.9864379 | 1.026847 |
| TG 54:8-FA22:6 | 2.1421981 | 4.9847472 | 0.9508513 | 4.8066582 | 6.8808002 | 1.0250043 | 0.808067 | 1.158411 | 0.7123751 |
| TG 55:1-FA16:0 | 16.673952 | 24.692637 | 6.1206674 | 26.322681 | 25.046664 | 7.8413438 | 8.2687812 | 9.897663 | 5.8282728 |
| TG 55:1-FA18:1 | 2.0046261 | 2.5714038 | 0.871321 | 3.6608967 | 3.9512601 | 0.722358 | 1.0148536 | 0.9345371 | 1.0589913 |
| TG 55:2-FA18:1 | 1.4513383 | 2.0114547 | 0.6122664 | 1.0660506 | 1.1849339 | 0.4661685 | 0.5534987 | 0.4696796 | 0.4406161 |
| TG 55:2-FA18:2 | 3.0314747 | 3.6066674 | 1.13463 | 7.8327739 | 4.270694 | 1.0128494 | 1.9291381 | 1.8269053 | 1.4291506 |
| TG 55:3-FA18:1 | 2.0585485 | 2.814377 | 1.1647276 | 1.3363087 | 2.6337419 | 0.987048 | 0.5904453 | 0.5790182 | 1.0890547 |
| TG 55:3-FA18:2 | 2.511220 | 3.5595945 | 0.796842 | 2.5099295 | 2.0181837 | 0.8022635 | 0.8599273 | 0.7517182 | 0.7587058 |
| TG 55:4-FA18:1 | 3.7527697 | 3.3832971 | 2.3590823 | 2.1735324 | 3.6367018 | 1.8041542 | 0.6854039 | 0.714954 | 2.2224567 |
| TG 55:4-FA18:2 | 3.3466396 | 4.1347751 | 1.2629397 | 2.5961986 | 3.0098183 | 1.121758 | 0.8933873 | 0.8423278 | 1.4628901 |
| TG 55:5-FA18:1 | 4.0614087 | 2.9353514 | 1.9421398 | 2.8067344 | 2.4972663 | 1.4028093 | 0.5887973 | 0.6420612 | 2.0839022 |
| TG 55:5-FA18:2 | 4.9397474 | 4.0363189 | 2.2579073 | 3.9920769 | 3.2899817 | 1.8671078 | 0.8447279 | 0.9038996 | 2.3567748 |
| TG 55:5-FA20:4 | 0.3302844 | 0.574931 | 0.1901396 | 0.2651892 | 0.5856735 | 0.1189662 | 0.1322212 | 0.1392757 | 0.1591757 |
| TG 55:7-FA15:0 | 0.2212666 | 0.3976608 | | 0.3468516 | 0.3599802 | 0.1129848 | 0.0988586 | 0.1336245 | |
| TG 55:7-FA22:6 | 1.1081466 | 2.3657445 | 0.4021075 | 1.1792953 | 2.5595443 | 0.5102569 | 0.4393716 | 0.5066596 | 0.4626312 |
| TG 56:1-FA16:0 | 1.4873978 | 1.8928984 | 0.7404946 | 1.2350537 | 0.8903044 | 0.7946212 | 0.3936575 | 0.4270701 | 0.6429287 |
| TG 56:1-FA18:1 | 0.624814 | 1.0397617 | 0.2045769 | 0.5154551 | 0.7582189 | 0.2576718 | 0.1892068 | 0.2059129 | 0.2302682 |
| TG 56:10-FA18:2 | 1.9609387 | 2.6477014 | 0.6516653 | 2.2223259 | 1.7419379 | 0.5544194 | 0.7490015 | 0.6138365 | 0.6935899 |
| TG 56:2-FA16:0 | 1.2170983 | 2.1679236 | 0.2895376 | 0.6242908 | 0.6258222 | 0.3457348 | 0.1941037 | 0.3596159 | 0.2359092 |
| TG 56:2-FA18:0 | 0.8870827 | 1.5418278 | 0.3626133 | 0.5020211 | 0.574489 | 0.3405267 | 0.2340222 | 0.2490558 | 0.2767513 |
| TG 56:2-FA20:0 | 2.5500408 | 3.5914546 | 1.3022526 | 2.0165246 | 3.3445771 | 1.2001435 | 0.6545728 | 0.7506793 | 1.2848604 |
| TG 56:2-FA20:1 | 2.648985 | 4.2210498 | 0.5505775 | 1.1996765 | 1.160877 | 0.4969401 | 0.3514387 | 0.7798893 | 0.5338758 |
| TG 56:3-FA16:0 | 0.9105823 | 1.4461297 | 0.2116398 | 0.464486 | 0.5244347 | 0.2693551 | 0.1623947 | 0.222974 | 0.202465 |
| TG 56:3-FA18:0 | 1.9318735 | 3.2811 | 0.5479222 | 1.1180007 | 1.179856 | 0.6370235 | 0.4687881 | 0.4471762 | 0.7172069 |
| TG 56:3-FA18:1 | 4.6274989 | 6.0861956 | 1.6000769 | 2.6573695 | 4.4296819 | 1.5079489 | 0.940014 | 1.0045348 | 1.4092503 |
| TG 56:3-FA18:2 | 2.8018919 | 4.8026342 | 0.6145379 | 1.9227805 | 1.7274161 | 0.7416043 | 0.5998386 | 0.6294855 | 0.6358615 |
| TG 56:3-FA20:0 | 4.5001211 | 5.2312658 | 1.708834 | 4.3856973 | 4.3443422 | 1.3589295 | 1.0566043 | 1.1668834 | 1.7635889 |
| TG 56:3-FA20:1 | 3.7385964 | 5.6570308 | 1.3166761 | 2.2495731 | 3.4386652 | 1.1320697 | 0.7665379 | 0.835448 | 1.0458875 |
| TG 56:3-FA20:2 | 0.2011395 | 0.2684299 | 0.1232722 | 0.1190946 | 0.2250982 | 0.1007324 | | 0.0757416 | 0.0814904 |
| TG 56:4-FA16:0 | 0.4111705 | 0.7109103 | 0.1475606 | 0.2735038 | 0.450995 | 0.2113663 | 0.1236598 | 0.1321409 | 0.1152383 |
| TG 56:4-FA18:0 | 0.5871103 | 0.8438946 | 0.2338903 | 0.4161466 | 1.0771313 | 0.3109006 | 0.2125589 | 0.2517296 | 0.2422069 |
| TG 56:4-FA18:1 | 7.7412695 | 9.7747716 | 2.1603457 | 4.736893 | 8.7015154 | 2.1178558 | 1.3726487 | 1.4207798 | 2.0212608 |
| TG 56:4-FA18:2 | 7.8525148 | 9.7788444 | 1.776414 | 5.5333034 | 6.1978263 | 1.7606943 | 1.5216976 | 1.4217773 | 1.7117776 |
| TG 56:4-FA20:1 | 7.4709342 | 9.1312534 | 1.974633 | 4.8233346 | 6.7580106 | 1.6391703 | 1.3305679 | 1.3724387 | 1.7641541 |
| TG 56:4-FA20:2 | 0.5233219 | 0.974561 | 0.2324376 | 0.4265846 | 1.7710051 | 0.2876604 | 0.1987003 | 0.1937036 | 0.2263198 |
| TG 56:4-FA20:3 | 0.3417373 | 0.5540104 | 0.1751238 | 0.2752604 | 0.6983881 | 0.2068783 | 0.1161093 | 0.1389206 | 0.1448813 |
| TG 56:4-FA20:4 | 0.254579 | 0.3559325 | 0.0791209 | 0.1824253 | 0.337315 | 0.0584988 | 0.0696787 | 0.1244396 | 0.1026317 |
| TG 56:4-FA22:4 | 0.0854179 | 0.0907876 | 0.0493122 | 0.0844349 | 0.096461 | 0.0348786 | | 0.0400482 | 0.0293549 |
| TG 56:5-FA16:0 | 0.7159145 | 1.3184153 | 0.2469534 | 0.4739002 | 1.9804356 | 0.3662251 | 0.2166153 | 0.2601047 | 0.2204028 |
| TG 56:5-FA18:0 | 0.8850524 | 1.4439586 | 0.4258863 | 1.1080392 | 1.8825102 | 0.4342828 | 0.4192741 | 0.5873421 | 0.458429 |
| TG 56:5-FA18:1 | 2.6310314 | 3.682988 | 1.052909 | 2.5325898 | 8.348018 | 1.0136317 | 0.8166999 | 0.8700802 | 0.8900812 |
| TG 56:5-FA18:2 | 9.9259854 | 12.258039 | 2.1123649 | 8.1252094 | 9.686219 | 2.0995214 | 1.9581978 | 1.6748736 | 2.0862145 |
| TG 56:5-FA20:1 | 6.2969411 | 7.8934648 | 1.2681884 | 5.0557746 | 5.1305765 | 1.1025066 | 1.1656037 | 1.050095 | 1.2334813 |
| TG 56:5-FA20:2 | 1.0147257 | 1.3101541 | 0.4005251 | 1.0919399 | 2.8311847 | 0.4105291 | 0.2924457 | 0.3261276 | 0.3734059 |
| TG 56:5-FA20:3 | 0.9847249 | 1.3787611 | 0.2999353 | 0.9346859 | 3.4458898 | 0.4013768 | 0.2985394 | 0.2954675 | 0.3548246 |
| TG 56:5-FA20:4 | 1.5766593 | 2.5997172 | 0.7336081 | 1.6720431 | 3.42153 | 0.6197983 | 0.7570365 | 1.0137477 | 0.7270278 |
| TG 56:5-FA22:4 | 0.7154601 | 1.5275287 | 0.3168577 | 0.5934767 | 2.6199819 | 0.3939148 | 0.2040218 | 0.3546272 | 0.2892052 |
| TG 56:5-FA22:5 | 0.1729507 | 0.5033897 | 0.1263558 | 0.1883194 | 0.5140197 | 0.1416765 | 0.0670676 | 0.1349828 | 0.1025994 |
| TG 56:6-FA16:0 | 3.0376066 | 5.2635223 | 1.4508586 | 4.2939154 | 7.0451538 | 2.1127163 | 1.2713407 | 2.1002805 | 1.3182833 |
| TG 56:6-FA18:0 | 1.2032618 | 1.6741384 | 0.4518471 | 1.5089974 | 2.323855 | 0.4586946 | 0.5863147 | 0.7211207 | 0.5787229 |
| TG 56:6-FA18:1 | 3.8587114 | 5.1529436 | 1.5634172 | 4.8416067 | 10.530497 | 1.7801753 | 1.2448844 | 1.9318219 | 1.4788141 |
| TG 56:6-FA18:2 | 4.2610113 | 5.0500682 | 1.2477612 | 4.7572033 | 8.3195426 | 1.2733473 | 1.3760635 | 1.5069401 | 1.2980081 |
| TG 56:6-FA18:3 | 0.8806479 | 1.0833858 | 0.2854349 | 0.8758409 | 0.864574 | 0.2003903 | 0.2030821 | 0.2196675 | 0.272856 |
| TG 56:6-FA20:2 | 0.9424119 | 0.9800017 | 0.2544168 | 1.2925352 | 1.7744176 | 0.2958325 | 0.2671841 | 0.3089898 | 0.2288498 |
| TG 56:6-FA20:3 | 1.8086511 | 2.0310587 | 0.5886076 | 2.0038986 | 5.4617957 | 0.5496306 | 0.597933 | 0.4018371 | 0.5571395 |
| TG 56:6-FA20:4 | 3.7740665 | 5.1926423 | 1.4647051 | 4.721942 | 9.9381432 | 1.2547935 | 1.8971065 | 2.0533189 | 1.6818709 |
| TG 56:6-FA20:5 | 0.6789722 | 1.0089568 | 0.2969792 | 0.7613673 | 0.6995549 | 0.2950887 | 0.2559368 | 0.3419274 | 0.3809835 |
| TG 56:6-FA22:4 | 1.5850258 | 2.6599075 | 0.7115769 | 1.5189651 | 3.2724433 | 0.7350663 | 0.604392 | 0.7748068 | 0.56615 |
| TG 56:6-FA22:5 | 4.0254463 | 6.6417263 | 1.8592347 | 5.4353449 | 9.6477808 | 2.8290148 | 1.4608326 | 2.4836942 | 1.7384518 |
| TG 56:6-FA22:6 | 0.9734636 | 2.5949511 | 0.6436185 | 1.0467279 | 2.2521113 | 0.751504 | 0.726655 | 0.8444883 | 0.5912649 |
| TG 56:7-FA16:0 | 9.1512296 | 16.267969 | 3.9150124 | 13.524038 | 21.564334 | 5.9515874 | 3.744312 | 5.9906689 | 4.0714884 |
| TG 56:7-FA16:1 | 0.2327158 | 0.4763302 | 0.1272221 | 0.3246365 | 0.905634 | 0.1054186 | 0.0911294 | 0.1134666 | 0.0907182 |
| TG 56:7-FA18:0 | 0.5086872 | 0.7465524 | 0.2060841 | 0.7236606 | 0.6320384 | 0.1752225 | 0.2967104 | 0.2837779 | 0.2464262 |
| TG 56:7-FA18:1 | 6.9363519 | 11.785773 | 3.0229165 | 8.5243438 | 18.866143 | 3.7722426 | 3.0024018 | 3.8786903 | 2.9844859 |
| TG 56:7-FA18:2 | 6.4038259 | 7.4156788 | 2.1337841 | 11.45476 | 10.868246 | 3.0951475 | 2.7829435 | 3.4420409 | 2.2313845 |
| TG 56:7-FA18:3 | 0.5589286 | 0.5848945 | 0.219443 | 0.7708499 | 0.8847701 | 0.1894647 | 0.2355703 | 0.2203618 | 0.2262362 |
| TG 56:7-FA20:3 | 1.6266421 | 1.4388493 | 0.506354 | 2.3417753 | 3.2009773 | 0.3885511 | 0.5172406 | 0.4531918 | 0.4219786 |
| TG 56:7-FA20:4 | 4.0392485 | 4.8270857 | 1.5702726 | 4.6683317 | 10.320893 | 1.2728565 | 2.2024008 | 2.223826 | 1.7037889 |
| TG 56:7-FA20:5 | 2.427005 | 3.2150413 | 1.2578101 | 4.605896 | 4.7233667 | 1.1825602 | 1.4171166 | 1.5305505 | 1.719644 |
| TG 56:7-FA22:4 | 0.2545218 | 0.3589297 | 0.1285301 | 0.3841944 | 0.6163499 | 0.0966987 | 0.0932897 | 0.1157834 | 0.0759457 |
| TG 56:7-FA22:5 | 8.4207674 | 11.647462 | 3.490571 | 15.831353 | 12.419049 | 5.2629013 | 3.4261964 | 6.0206173 | 3.4113526 |
| TG 56:7-FA22:6 | 37.480199 | 71.838507 | 16.484036 | 44.396131 | 104.54731 | 23.699321 | 15.249527 | 22.207844 | 16.165728 |
| TG 56:8-FA16:0 | 17.481928 | 25.755504 | 6.2479496 | 28.29595 | 26.653948 | 8.6189046 | 8.501203 | 10.524562 | 6.2041971 |
| TG 56:8-FA16:1 | 1.0104419 | 1.812784 | 0.4082954 | 1.613263 | 3.2686912 | 0.4641719 | 0.2729016 | 0.4531839 | 0.4152545 |
| TG 56:8-FA18:1 | 1.9921016 | 2.7214048 | 1.0372561 | 4.1355918 | 4.2201693 | 0.8142325 | 1.075068 | 1.1093055 | 1.1591166 |
| TG 56:8-FA18:2 | 14.965107 | 20.456987 | 5.3367469 | 25.066304 | 23.442156 | 6.7880597 | 7.4691835 | 8.8421565 | 5.4891481 |
| TG 56:8-FA18:3 | 0.6749341 | 0.8419695 | 0.3749543 | 1.2912442 | 0.9819533 | 0.3422449 | 0.3253952 | 0.3853738 | 0.4598481 |
| TG 56:8-FA20:4 | 3.2869421 | 3.6549752 | 1.0353387 | 4.8629007 | 6.8458501 | 0.9195011 | 2.0882289 | 1.851915 | 1.15913 |
| TG 56:8-FA20:5 | 4.8410429 | 4.3609643 | 2.2408186 | 12.186221 | 6.7871713 | 1.8137547 | 2.8601747 | 3.0162354 | 3.0602105 |
| TG 56:8-FA22:5 | 1.7908295 | 2.4965627 | 0.7386527 | 3.304832 | 2.9426763 | 0.7515887 | 0.7695251 | 0.9132317 | 0.7367458 |
| TG 56:8-FA22:6 | 87.438101 | 133.21554 | 32.156357 | 144.02202 | 141.88085 | 43.66055 | 42.260405 | 52.23143 | 31.447729 |
| TG 56:9-FA18:3 | 0.7498126 | 0.8873845 | 0.3052722 | 1.7548187 | 1.1575231 | 0.2620616 | 0.4409529 | 0.4924434 | 0.3691867 |
| TG 56:9-FA20:4 | 0.7186468 | 0.7340935 | 0.269257 | 1.3898308 | 0.9583947 | 0.1852792 | 0.4948824 | 0.4277223 | 0.2969902 |
| TG 56:9-FA20:5 | 4.5520409 | 3.4848153 | 1.7973331 | 14.453881 | 4.2891388 | 1.3336767 | 3.1691505 | 2.912451 | 2.4825752 |
| TG 56:9-FA22:6 | 8.1434416 | 12.735075 | 3.4025847 | 15.642339 | 17.78689 | 3.1414011 | 3.0819116 | 3.7439769 | 3.0535839 |
| TG 57:10-FA22:6 | 0.5412725 | 0.6251503 | 0.2947949 | 0.7490027 | 0.6923886 | 0.2936681 | 0.4346307 | 0.4908074 | 0.3616793 |
| TG 57:2-FA18:1 | 3.721137 | 5.2119906 | 1.3624439 | 6.057984 | 9.4264082 | 1.7629437 | 1.7015253 | 1.6903471 | 1.5500499 |
| TG 57:3-FA18:2 | 5.5987165 | 6.2801785 | 1.4527449 | 11.510067 | 8.641119 | 1.8316734 | 3.103455 | 2.4948367 | 1.8371371 |
| TG 58:10-FA18:2 | 6.0407753 | 6.8646482 | 1.6451588 | 12.198196 | 9.0335727 | 2.1901212 | 3.3529384 | 2.7756086 | 1.9817602 |
| TG 58:10-FA20:4 | 0.7443165 | 0.9841477 | 0.3279794 | 0.9892417 | 1.5983827 | 0.382375 | 1.2165959 | 0.9712472 | 0.5319373 |
| TG 58:10-FA20:5 | 0.547457 | 0.5556516 | 0.299917 | 1.2429878 | 0.5962803 | 0.271418 | 0.8662163 | 0.699298 | 0.422865 |
| TG 58:10-FA22:5 | 0.7161937 | 0.6897743 | 0.2315149 | 1.6530266 | 0.7501118 | 0.2526406 | 0.3163149 | 0.3223102 | 0.2215007 |
| TG 58:10-FA22:6 | 16.464956 | 18.660486 | 4.5373411 | 35.369989 | 28.606802 | 6.1555549 | 8.9849664 | 7.8462384 | 5.4737525 |
| TG 58:2-FA18:1 | 0.4915727 | 0.7846022 | 0.2048294 | 0.3793516 | 0.4265745 | 0.203343 | 0.1272409 | 0.1648197 | 0.1879674 |
| TG 58:3-FA18:1 | 0.8698818 | 1.2812091 | 0.1740012 | 0.4698643 | 0.4947639 | 0.2326443 | 0.1641794 | 0.271079 | 0.1765642 |
| TG 58:5-FA18:1 | 0.2233735 | 0.332688 | 0.146642 | 0.1777312 | 0.395767 | 0.1246785 | 0.0999417 | 0.0819212 | 0.0939933 |
| TG 58:6-FA16:0 | 0.2724846 | 0.4475274 | 0.181177 | 0.2171949 | 0.3068598 | 0.2176481 | 0.0778283 | 0.1127896 | 0.1340095 |
| TG 58:6-FA18:0 | 0.1739864 | 0.3068491 | 0.0757111 | 0.2073615 | 0.3826111 | 0.0870918 | 0.0857966 | 0.1096973 | 0.0767768 |
| TG 58:6-FA18:1 | 0.3438223 | 0.5448021 | 0.1530345 | 0.3291334 | 1.1694083 | 0.1776263 | 0.1238625 | 0.1277343 | 0.1211596 |
| TG 58:6-FA20:4 | 0.3262422 | 0.4873101 | | 0.3405548 | 0.3343586 | 0.1316785 | 0.1939291 | 0.1074326 | 0.0710855 |
| TG 58:6-FA22:4 | 0.2136403 | 0.3791287 | 0.104951 | 0.202068 | 0.8792251 | 0.1456562 | 0.1212736 | 0.0755035 | 0.0956114 |
| TG 58:6-FA22:5 | 0.3578078 | 0.5692669 | 0.187464 | 0.4442618 | 0.5157728 | 0.2438624 | 0.1525418 | 0.140801 | 0.1789106 |
| TG 58:7-FA16:0 | 0.8481965 | 1.4958569 | 0.3826946 | 0.6459787 | 1.4341209 | 0.5826218 | 0.2857516 | 0.3758871 | 0.3711798 |
| TG 58:7-FA18:0 | 0.439661 | 1.0509923 | 0.2562622 | 0.8114776 | 1.3612326 | 0.2670475 | 0.3420364 | 0.3282548 | 0.2999611 |
| TG 58:7-FA18:1 | 0.9701299 | 1.7734423 | 0.4172027 | 1.1815469 | 3.4447834 | 0.6450355 | 0.4585327 | 0.4182805 | 0.4260302 |
| TG 58:7-FA18:2 | 0.5189167 | 0.8269952 | 0.2110786 | 0.7065178 | 1.2154703 | 0.2498218 | 0.2773006 | 0.2802349 | 0.2034666 |
| TG 58:7-FA20:4 | 0.4429659 | 0.6592516 | 0.1037578 | 0.3196235 | 0.7133405 | 0.1686659 | 0.2196896 | 0.1678079 | 0.1266462 |
| TG 58:7-FA22:4 | 0.402953 | 0.6260496 | 0.1364911 | 0.3999361 | 1.2142933 | 0.2147379 | 0.2023283 | 0.1914459 | 0.1987583 |
| TG 58:7-FA22:5 | 0.8913796 | 1.5873407 | 0.4543144 | 1.4399028 | 2.7478045 | 0.5646335 | 0.4284176 | 0.4690189 | 0.4608296 |
| TG 58:7-FA22:6 | 2.3855442 | 4.3810055 | 1.2570878 | 2.4977723 | 5.0090873 | 1.5916641 | 1.0836651 | 1.0310987 | 1.1848672 |
| TG 58:8-FA18:1 | 2.6066086 | 4.1284678 | 1.125656 | 4.2775886 | 9.7837014 | 1.5523221 | 1.1263048 | 1.1570483 | 1.3043005 |
| TG 58:8-FA18:2 | 1.9334659 | 3.0474374 | 0.7650057 | 3.1211106 | 4.3189705 | 0.9896138 | 1.0104506 | 1.0821059 | 0.800028 |
| TG 58:8-FA20:3 | 0.1677901 | 0.2541841 | | 0.196376 | 0.3975989 | 0.0941494 | 0.1171342 | 0.0950124 | |
| TG 58:8-FA20:4 | 0.3088312 | 0.4068403 | 0.105143 | 0.3569714 | 0.8093361 | 0.0815241 | 0.2635819 | 0.2542554 | 0.1253996 |
| TG 58:8-FA22:5 | 1.8864812 | 2.1820051 | 0.7797 | 3.5509729 | 4.0507375 | 0.9407732 | 0.7581512 | 0.7417205 | 0.7975655 |
| TG 58:8-FA22:6 | 9.5148151 | 18.038359 | 4.091772 | 13.413597 | 35.407143 | 5.7470057 | 5.0613691 | 4.94878 | 4.9797705 |
| TG 58:9-FA18:1 | 3.7857645 | 5.2360887 | 1.3365225 | 6.2982755 | 10.059276 | 1.7878836 | 1.8925984 | 1.7305422 | 1.70379 |
| TG 58:9-FA18:2 | 3.4280722 | 4.5201578 | 1.0638803 | 6.7077786 | 8.3368096 | 1.6414707 | 1.7516986 | 1.7138305 | 1.2109481 |
| TG 58:9-FA20:4 | 0.4140332 | 0.4785991 | 0.1692385 | 0.5116916 | 0.8275501 | 0.1080121 | 0.4885221 | 0.3556605 | 0.1798363 |
| TG 58:9-FA22:5 | 1.4697584 | 1.5501169 | 0.5380027 | 3.2422222 | 2.082629 | 0.65112 | 0.8806829 | 0.7063165 | 0.4935449 |
| TG 58:9-FA22:6 | 18.61141 | 25.434474 | 6.8474466 | 32.810368 | 51.1683 | 9.2347201 | 8.7648203 | 8.7361836 | 8.0187712 |
| TG 60:10-FA22:5 | 0.2183027 | 0.2841845 | 0.0983874 | 0.3200879 | 0.380738 | 0.1137942 | 0.1528195 | 0.1360617 | 0.0888765 |
| TG 60:10-FA22:6 | 1.0561734 | 2.0894851 | 0.3015817 | 1.8203787 | 3.0572719 | 0.5322239 | 0.8369942 | 0.611418 | 0.3864341 |
| TG 60:11-FA22:5 | 0.2662207 | 0.5024728 | 0.1331586 | 0.5776475 | 0.5082359 | 0.276021 | 0.2841996 | 0.2693696 | 0.131211 |
| TG 60:11-FA22:6 | 1.6567391 | 2.5009244 | 0.5292205 | 2.5558195 | 3.7683435 | 1.0816952 | 1.4808636 | 1.3265684 | 0.6103937 |
| TG 60:12-FA22:6 | 3.0208612 | 5.4920393 | 1.2175371 | 5.6302075 | 5.7262161 | 2.4380652 | 4.4395697 | 3.6967996 | 1.6932683 |

| | Lipid species | | | | | | | | | |
|---|---|---|---|---|---|---|---|---|---|---|
| | Bt:BSHIVMOL361- | Bt:BSHIVMOL361- | Bt:BSHIMOL361- | Bt:BSHIMOL361- | Bt:BSHIMOL361- | Bt:BSHIMOL361- | Bt:BSHIII1E2- | Bt:BSHIII1E2- | Bt:BSHIII1E2- | Bt:BSHIII1E2- |
| | F16 | F17 | M12 | M13 | F14 | F15 | M8 | M9 | M10 | F11 |
| CE 12:0 | 3.6580931 | 0.7926343 | 1.7960596 | 2.57589 | 2.04201 | 1.040752 | 1.9176849 | 2.1969315 | 2.6354707 | 2.1786693 |
| CE 14:0 | 6.3043649 | 4.3469295 | 5.194435 | 5.7531597 | 4.6540089 | 3.0124468 | 4.6318943 | 4.631584 | 6.5477518 | 5.6069791 |
| CE 14:1 | 5.4931365 | 1.428596 | 3.8780368 | 4.8809786 | 3.8167046 | 1.5069648 | 3.7409554 | 3.3142005 | 5.4648106 | 4.1912094 |
| CE 15:0 | 8.4737029 | 4.2921358 | 6.5285139 | 7.1218766 | 5.6132151 | 3.1488501 | 5.1701132 | 5.5277658 | 7.8536281 | 6.618575 |
| CE 16:0 | 86.697314 | 93.028925 | 82.522357 | 72.835394 | 66.546174 | 57.003381 | 75.191678 | 77.980515 | 96.991981 | 93.958926 |
| CE 16:1 | 37.124467 | 75.36668 | 35.297183 | 28.753677 | 32.103473 | 31.435352 | 28.824514 | 31.737744 | 43.024515 | 54.315762 |
| CE 17:0 | 4.9121194 | 6.4055141 | 4.478475 | 4.2361353 | 5.1113337 | 3.2368795 | 4.9879889 | 3.3624951 | 5.5219285 | 5.5697897 |
| CE 18:0 | 5.8917728 | 10.750232 | 7.091118 | 5.0185987 | 6.7592949 | 5.354442 | 7.1248578 | 5.0672502 | 7.6702673 | 6.8187665 |
| CE 18:1 | 101.1598 | 287.62788 | 95.288424 | 80.549954 | 109.17137 | 102.33843 | 92.200228 | 83.878146 | 110.2383 | 146.7972 |
| CE 18:2 | 1267.6847 | 830.43583 | 1060.3564 | 1069.6102 | 758.08954 | 637.5834 | 1003.655 | 1204.1864 | 1387.679 | 1031.6019 |
| CE 18:3 | 33.27735 | 18.729852 | 28.037208 | 29.596903 | 25.519152 | 14.943774 | 24.964467 | 25.140949 | 31.879268 | 27.35684 |
| CE 18:4 | 3.5372114 | 1.3513208 | 2.6915035 | 2.9628196 | 2.8185187 | 1.1186722 | 2.0843674 | 2.1966391 | 2.9149185 | 2.4560804 |
| CE 20:0 | 293.25724 | 70.894211 | 168.41202 | 230.76558 | 157.76391 | 87.485047 | 191.09143 | 213.17406 | 221.90221 | 152.60485 |
| CE 20:1 | 98.611196 | 9.2536879 | 37.123673 | 78.339815 | 68.591309 | 14.818806 | 45.505548 | 38.828437 | 62.669178 | 47.787338 |
| CE 20:2 | 3.7363241 | 1.8069044 | 2.2422702 | 2.8965331 | 3.1747882 | 1.16447 | 2.0049278 | 1.9008074 | 3.0704116 | 2.3686309 |
| CE 20:3 | 24.546141 | 26.273581 | 25.433467 | 21.885898 | 16.139361 | 17.026909 | 18.852254 | 26.527992 | 24.463773 | 26.224151 |
| CE 20:4 | 607.89027 | 450.80356 | 629.0342 | 531.03167 | 448.06024 | 412.74661 | 471.11813 | 634.81841 | 545.16936 | 606.32979 |
| CE 20:5 | 125.27131 | 113.09258 | 106.38496 | 93.912109 | 80.836746 | 80.43222 | 102.33489 | 126.71416 | 171.66043 | 110.74972 |
| CE 22:0 | 5.3044209 | 2.1318532 | 3.4036909 | 3.9051104 | 2.8243329 | 1.8267582 | 3.1908917 | 3.280439 | 5.2724594 | 4.106103 |
| CE 22:1 | 4.6923709 | 1.8849572 | 3.647789 | 3.4210455 | 2.6266453 | 1.8244574 | 3.1386418 | 3.7874231 | 3.6064038 | 3.3537578 |
| CE 22:2 | 42.29661 | 15.099143 | 30.864415 | 34.258432 | 25.547622 | 20.318353 | 30.250667 | 39.959151 | 26.648645 | 28.259681 |
| CE 22:4 | 6.8902838 | 1.6640183 | 4.6080697 | 5.6176147 | 4.5479532 | 2.4437434 | 4.0020705 | 4.2454138 | 4.8185017 | 4.7486835 |
| CE 22:5 | 11.422316 | 7.5240562 | 8.3235345 | 6.7000833 | 6.145526 | 4.7610453 | 7.2207766 | 6.9008213 | 12.195846 | 8.1537734 |
| CE 22:6 | 194.24443 | 137.73733 | 159.81215 | 147.82835 | 98.604478 | 96.396379 | 162.39625 | 197.64689 | 232.05809 | 170.25507 |
| CE 24:0 | 84.897984 | 36.092907 | 73.169007 | 69.870603 | 42.816916 | 49.410016 | 69.308005 | 90.260421 | 60.254399 | 62.771104 |
| CE 24:1 | 19.021779 | 8.597845 | 12.596927 | 14.395107 | 9.8927974 | 9.1992214 | 13.547718 | 16.339368 | 17.479622 | 12.193813 |
| Cer d18:0/24:1 | | | | | | 0.2680448 | | | | |
| Cer d18:1/14:0 | | | | | | | | | | |
| Cer d18:1/16:0 | 0.1676792 | 0.1681277 | 0.2002354 | 0.1778481 | 0.1908725 | 0.14377 | 0.1488977 | 0.1177151 | 0.2230323 | 0.2320636 |
| Cer d18:1/18:0 | | | | | | 0.0625459 | | | | |
| Cer d18:1/20:0 | | | | | | | | | | |
| Cer d18:1/22:0 | 1.0440365 | 0.3318669 | 0.8721007 | 0.8962401 | 0.3683281 | 0.4566125 | 0.7791693 | 0.9414164 | 0.9439177 | 0.3223077 |
| Cer d18:1/24:0 | 1.370194 | 0.7730831 | 1.0973682 | 1.1094032 | 0.7398598 | 0.8220561 | 0.7670144 | 0.8169985 | 1.2297482 | 0.7758518 |
| Cer d18:1/24:1 | 0.4898566 | 0.9129086 | 0.3556753 | 0.4892119 | 0.7715835 | 0.8537345 | 0.2651462 | 0.3609655 | 0.4095098 | 0.698685 |
| DG 12:0_18:1 | | | 0.2684882 | | | | | | | |
| DG 12:0_18:2 | | | | 0.5764418 | | | | | | |
| DG 14:1_18:1 | | | | | | | | | | |
| DG 15:0_18:2 | | | | | | | | | | |
| DG 16:0_16:0 | 0.5712101 | 0.3870702 | 0.557242 | 0.556452 | 0.7600047 | 0.318855 | 0.4864776 | 0.3954026 | 0.4706918 | 0.5476047 |
| DG 16:0_16:1 | | | | | | | | | | |
| DG 16:0_18:0 | | | | | | | 1.7650747 | | | |
| DG 16:0_18:1 | 2.5817944 | 1.8087951 | 1.6630539 | 1.7503957 | 2.6014169 | 1.4336315 | 1.3563894 | 1.6105319 | 1.6506871 | 3.0576643 |
| DG 16:0_18:2 | | 2.763573 | | 6.7373682 | 6.1725741 | 4.461057 | 7.4621215 | 5.6226368 | | 4.3826092 |
| DG 16:0_22:6 | | | | | | | | 0.4194765 | | |
| DG 16:1_18:1 | | 0.3497686 | | | 0.3725159 | | | | | |
| DG 16:1_18:2 | | 0.8462362 | | 0.8885846 | 1.2487553 | 0.9681309 | 0.9921304 | 0.6286693 | | 1.0086021 |
| DG 18:0_18:1 | | 0.1628333 | | | 0.2658465 | | 0.1989183 | | | 0.233913 |
| DG 18:0_18:2 | | | | 1.021402 | 1.0614685 | 0.6156215 | | 0.7244871 | | 0.6044455 |
| DG 18:1_18:1 | 1.1659992 | 2.571286 | 0.67396 | 0.8076498 | 1.4789625 | 1.1393391 | 0.3255856 | 0.5882894 | 0.4953021 | 2.2041557 |
| DG 18:1_18:2 | | 11.767728 | | 12.888031 | 14.264311 | 10.309941 | 7.8685218 | 6.6256629 | | 11.599958 |
| DG 18:1_20:1 | 0.7615155 | 0.3272946 | 0.3971991 | 0.461825 | 0.6434222 | | 0.304779 | | 0.3728777 | 0.4003463 |
| DG 18:1_20:2 | | | | | 0.3575817 | | | | | |
| DG 18:1_20:4 | | | | | | | | | | |
| DG 18:1_22:6 | | | | | | | | 1.6471962 | | |
| DG 18:2_20:4 | | | | | | | | | | |
| DG 18:2_22:6 | | | | | | | | 4.5682117 | | |
| FA 12:0 | 3.0765737 | 2.4071725 | 2.4313353 | 2.9397414 | 2.9532835 | 2.5286224 | 3.0637831 | 3.0790238 | 2.8637477 | 2.7854558 |
| FA 14:0 | 20.523373 | 17.065264 | 12.865227 | 15.38645 | 20.804251 | 17.778222 | 26.605446 | 28.019273 | 21.245046 | 19.526657 |
| FA 14:1 | 5.1995161 | 4.7730261 | 3.7864972 | 4.289778 | 5.7932388 | 4.8873679 | 5.5351547 | 5.3326564 | 5.222874 | 5.0487987 |
| FA 15:0 | 10.36534 | 9.2947429 | 8.2764875 | 10.374332 | 11.79886 | 9.3556365 | 10.030368 | 10.33425 | 10.273701 | 10.159974 |
| FA 16:0 | 183.30583 | 166.71131 | 125.55464 | 163.86842 | 203.49007 | 173.56772 | 211.81081 | 221.50856 | 176.57827 | 170.29609 |
| FA 16:1 | 23.762066 | 26.20237 | 10.661124 | 13.60639 | 31.521746 | 24.331187 | 28.120801 | 39.626388 | 29.850312 | 20.03358 |
| FA 17:0 | 11.9777 | 10.570495 | 9.9958562 | 11.396243 | 12.170755 | 10.933287 | 12.04026 | 11.957345 | 11.753945 | 11.358477 |
| FA 18:0 | 59.354454 | 52.000069 | 47.370458 | 53.153872 | 55.126953 | 59.505681 | 65.130875 | 57.415002 | 54.764074 | 62.442264 |
| FA 18:1 | 62.360039 | 81.528129 | 35.037085 | 60.372756 | 81.12321 | 62.800084 | 68.792003 | 81.292694 | 60.594264 | 60.105126 |
| FA 18:2 | 72.014212 | 76.806012 | 32.442462 | 77.954509 | 89.945888 | 69.455117 | 78.509594 | 91.859816 | 66.509182 | 57.718272 |
| FA 18:3 | 6.4524666 | 7.0145897 | 2.9971291 | 4.4400662 | 7.2872924 | 6.7761397 | 7.8615472 | 8.6770736 | 6.8640983 | 4.8221046 |
| FA 18:4 | 1.3262884 | 1.105855 | 0.9890403 | 1.2108543 | 1.3623268 | 1.1234179 | 1.3121911 | 1.3129347 | 1.3983354 | 1.1576514 |
| FA 20:0 | 8.5679416 | 4.0696908 | 3.5603511 | 9.4290572 | 10.879333 | 5.0637638 | 7.6278923 | 7.4830679 | 5.8673727 | 5.3437934 |
| FA 20:1 | 6.2749069 | 4.2482637 | 3.4668308 | 6.0933355 | 7.5439132 | 3.9293807 | 5.7086886 | 4.6328437 | 5.5422874 | 4.8251959 |
| FA 20:2 | 1.9387879 | 1.8942474 | 1.4136163 | 1.7169535 | 2.2545173 | 1.7535352 | 1.8250349 | 1.8397325 | 1.7781908 | 1.9354921 |
| FA 20:3 | 2.4150942 | 2.6064308 | 1.7434604 | 2.0979238 | 2.4409672 | 2.3088563 | 2.2266106 | 2.1660476 | 2.1168313 | 2.2886372 |
| FA 20:4 | 4.2194122 | 6.0761332 | 3.9807248 | 4.0208655 | 4.8814364 | 6.3522878 | 4.7518646 | 4.8138501 | 4.2635529 | 5.0042444 |
| FA 20:5 | 2.3163202 | 3.1105616 | 1.6617986 | 1.9873641 | 2.1368415 | 2.8682399 | 2.764051 | 2.893924 | 2.9987519 | 2.0892809 |
| FA 22:0 | 3.7878699 | 3.3429245 | 3.0516841 | 3.7086984 | 3.9577341 | 3.2151873 | 3.5731798 | 3.5188081 | 3.351363 | 3.2942523 |
| FA 22:1 | 4.0289646 | 3.716404 | 2.5125756 | 3.0888851 | 3.9459404 | 3.1873578 | 4.201535 | 3.850906 | 4.1551623 | 3.6520441 |
| FA 22:2 | 1.8244959 | 1.3700622 | 1.2897676 | 1.3666671 | 1.9659668 | 1.5385183 | 1.6673697 | 1.6141534 | 1.7616711 | 1.4055806 |
| FA 22:4 | 1.5564871 | 1.24563 | 1.3398408 | 1.489889 | 1.641898 | 1.3696849 | 1.4520715 | 1.3155625 | 1.3405505 | 1.3517495 |
| FA 22:5 | 5.1259684 | 4.5422486 | 4.2625721 | 3.8419076 | 5.6282373 | 5.1572067 | 5.4748127 | 4.3443007 | 5.0790853 | 4.8084123 |
| FA 22:6 | 5.1956156 | 8.6815076 | 2.9251658 | 4.4252042 | 5.4100177 | 7.4263467 | 7.4174999 | 7.8308759 | 7.446235 | 6.0905586 |
| FA 24:0 | 2.6152097 | 1.9277048 | 2.3511709 | 2.6651778 | 2.576732 | 2.2230309 | 2.4257816 | 2.2548609 | 2.3499037 | 2.20717 |
| FA 24:1 | 3.3949594 | 2.9015713 | 2.6833271 | 2.9730252 | 3.158639 | 2.9076692 | 3.5679356 | 3.0835102 | 3.156549 | 3.0244658 |
| HexCER d18:1/16:0 | 0.1433926 | 0.4157281 | 0.1797717 | 0.1588118 | 0.3290385 | 0.3568915 | 0.1162816 | 0.1574543 | 0.137549 | 0.3846875 |
| HexCER d18:1/18:0 | | 0.1216031 | 0.0800932 | | | 0.1508864 | | | | 0.1316511 |
| HexCER d18:1/20:0 | | 0.1436785 | | | | 0.1372593 | | | | |
| HexCER d18:1/22:0 | 0.4974887 | 0.6039758 | 0.8844851 | 0.4631636 | 0.4193398 | 0.4873431 | 0.5305222 | 0.6351245 | 0.6667591 | 0.4673697 |
| HexCER d18:1/22:1 | | 0.1258802 | | | | 0.1314413 | | | | 0.1343746 |
| HexCER d18:1/24:0 | 0.1662418 | 0.4039145 | 0.2681605 | 0.1043351 | 0.2077278 | 0.2821018 | | 0.1855005 | 0.1957696 | 0.2962405 |
| HexCER d18:1/24:1 | | 0.9458165 | 0.2405544 | 0.1718748 | 0.3624886 | 0.5879752 | 0.1374917 | 0.1467591 | 0.1656501 | 0.5580405 |
| LPC 15:0 | 0.8087539 | 0.7161131 | 1.0909031 | 1.0480987 | 0.7366124 | 0.7166251 | 0.8213379 | 0.8646513 | 0.8072747 | 0.7059636 |
| LPC 16:0 | 210.459 | 183.4655 | 209.70943 | 186.96787 | 158.78731 | 163.28073 | 176.69208 | 214.02287 | 190.77929 | 210.28766 |
| LPC 16:1 | 2.853036 | 3.9458268 | 2.537378 | 2.4032262 | 2.4513495 | 2.3295413 | 1.8537346 | 2.5135212 | 2.2528597 | 3.9259857 |
| LPC 17:0 | 6.1866704 | 4.4280282 | 5.6189308 | 5.3609307 | 4.3822451 | 4.186176 | 4.5386557 | 5.315034 | 5.4174288 | 4.2025201 |
| LPC 18:0 | 90.941607 | 69.197745 | 90.956311 | 80.701393 | 67.07233 | 82.882357 | 66.000921 | 85.912098 | 71.790395 | 77.981562 |
| LPC 18:1 | 38.544393 | 53.290049 | 34.502286 | 35.7588 | 34.257174 | 38.648738 | 25.027758 | 29.338975 | 30.861563 | 45.966879 |
| LPC 18:2 | 109.09439 | 115.00295 | 95.853595 | 132.43065 | 111.98777 | 116.41582 | 91.659617 | 98.948831 | 100.74379 | 110.43071 |
| LPC 18:3 | | 0.7728139 | 0.5323706 | | 0.4509649 | 0.6380145 | 0.5070811 | 0.6682318 | 0.5007071 | 0.7073542 |
| LPC 20:0 | 6.5794707 | 1.2656131 | 3.6700002 | 8.128901 | 7.518903 | 2.6582383 | 4.4762282 | 4.0307752 | 4.2335194 | 4.1804832 |
| LPC 20:1 | 1.7262188 | 1.1088566 | 1.5165859 | 1.9311471 | 1.489279 | 0.7380089 | 1.1571981 | 1.049474 | 1.339639 | 1.1263436 |
| LPC 20:2 | 0.8086499 | 1.3216116 | 0.9173786 | 0.863341 | 0.8374407 | 0.7021 | 0.5025703 | 0.9979613 | 0.642155 | 0.8985384 |
| LPC 20:3 | 4.5962309 | 8.2018881 | 4.0352509 | 4.5771997 | 4.017997 | 6.8606782 | 3.8272866 | 5.0449321 | 4.0910046 | 6.3142465 |
| LPC 20:4 | 9.0365704 | 14.45263 | 9.1306783 | 8.3590659 | 9.1345309 | 15.604274 | 7.9535482 | 10.240333 | 7.9563457 | 12.424621 |
| LPC 20:5 | 0.5898218 | 1.7461221 | 0.6180554 | 0.5784075 | 0.740707 | 1.0192701 | 0.565777 | 0.6527269 | 1.1091321 | 0.8651661 |
| LPC 22:5 | 0.5780179 | 0.7191528 | 0.4485606 | | 0.2725554 | 0.4918117 | 0.3013014 | | 0.4675566 | 0.4999281 |
| LPC 22:6 | 1.3500348 | 2.8765376 | 1.1681292 | 1.1684927 | 1.0821467 | 2.1281971 | 1.4504782 | 1.9858946 | 1.9108286 | 1.4854629 |
| LPE 16:0 | 4.6810324 | 6.0972489 | 5.4476681 | 5.0501378 | 4.3173204 | 4.4096701 | 4.2273483 | 5.4708223 | 4.4513589 | 7.7849908 |
| LPE 18:0 | 2.3215119 | 1.8722487 | 2.1558606 | 2.5681785 | 1.9079489 | 2.0958027 | 2.0164083 | 2.7585337 | 1.9595296 | 2.9932842 |
| LPE 18:1 | 0.9989941 | 1.9322763 | 0.6914244 | 1.4038919 | 0.850875 | 0.7954878 | 0.621274 | 0.8484891 | 0.7395341 | 1.7429649 |
| LPE 18:2 | 2.6138725 | 2.7180759 | 2.5490386 | 4.5987372 | 2.4704308 | 1.9425891 | 2.387143 | 2.5704965 | 3.3030248 | 3.5928205 |
| LPE 20:4 | | | | 0.4598686 | 0.3397829 | 0.4447572 | | 0.6006733 | | |
| LacCER d18:1/16:0 | | | | | | 0.1363377 | | | | |
| LacCER d18:1/18:1 | | | | 0.1583014 | | | | | | |
| LacCER d18:1/22:0 | | 0.17865 | | | | | 0.1563139 | | | |
| PA 34:1 | 0.5082822 | 0.2244102 | 0.4256072 | 0.3772869 | 0.3177437 | 0.2075854 | 0.2351402 | 0.2542215 | 0.3266569 | 0.5878362 |
| PA 34:2 | 2.1810296 | 0.5588633 | 1.4115386 | 1.8964838 | 1.065994 | 0.4538898 | 1.1496507 | 1.2100652 | 1.9397633 | 1.4102428 |
| PA 34:3 | | 0 | | | | | | | | |
| PA 34:4 | 2.2356091 | 2.4075688 | 2.3462308 | 2.3469755 | 2.1112698 | 2.14805 | 2.14579 | 2.2081776 | 2.4526118 | 2.098898 |
| PA 34:5 | 0.642644 | 0.5347679 | 0.5970138 | 0.6346145 | 0.4332346 | 0.5295902 | 0.5942495 | 0.6532716 | 0.6898923 | 0.6924651 |
| PA 36:1 | 0.3005941 | | 0.1112379 | 0.2236133 | 0.1082432 | | 0.1540723 | 0.1125565 | | 0.1568084 |
| PA 36:2 | 1.2816894 | 0.4109072 | 1.1866467 | 1.4274852 | 0.8590781 | 0.4976402 | 0.736643 | 0.8124928 | 1.1826387 | 1.0240413 |
| PA 36:3 | 0.5293344 | | 0.3423167 | 0.4357083 | 0.2678005 | 0.1775614 | | 0.2457407 | 0.3431248 | 0.4231869 |
| PA 36:4 | 0.2241544 | | | 0.2959157 | 0.1950945 | | 0.1629513 | 0.2283093 | 0.2121995 | 0.2137951 |
| PA 36:5 | 0.525146 | 0.2958845 | | 0.3531749 | 0.4106895 | 0.2197822 | 0.2901061 | 0.3070555 | 0.3753423 | 0.4317662 |
| PA 38:3 | | | | | | | 0.1226545 | | | |
| PA 38:4 | 0.2529423 | | 0.255327 | 0.2688831 | 0.228907 | 0.172656 | 0.3453189 | 0.2633033 | 0.3868748 | 0.273889 |
| PA 38:6 | 0.2971872 | | | | | | | | | |
| PA 38:7 | 0.1927222 | 0.2034586 | | 0.2119445 | | 0.1510916 | | 0.2070405 | 0.2998741 | 0.2564962 |
| PA 40:6 | | | | | 0.1340125 | | | 0.1523188 | | |
| PA 40:8 | | | | | | | | | | |
| PC 12:0_16:0 | 0.5669536 | | 0.4697966 | 0.5441006 | 0.4819114 | | 0.3928159 | 0.4829269 | 0.450916 | |
| PC 12:0_18:0 | | | | | | | | | | |
| PC 12:0_18:3 | 1.7131735 | 0.4304301 | 1.2992569 | 1.7165296 | 1.0448838 | | 0.6094429 | 0.759002 | 1.3212826 | 1.1234611 |
| PC 14:0_18:1 | 0.8203231 | 1.5195327 | 1.021673 | 0.6846992 | 0.598696 | 0.6273024 | 0.6189559 | 0.7151603 | 0.7275012 | 1.1639548 |
| PC 14:0_18:2 | 2.0085938 | 1.0233983 | 2.1206339 | 1.990674 | 1.271514 | 1.1451204 | 1.6843172 | 1.696496 | 2.1536339 | 1.8080882 |
| PC 14:0_18:3 | | | | | | | | | | |
| PC 14:0_20:4 | | | | | | | | | | 1.1820679 |
| PC 14:1_20:0 | | 0.3844417 | | | | | 0.43322 | | | 0.3497967 |
| PC 15:0_18:1 | 0.5388297 | 0.4496185 | 0.4652028 | 0.5147186 | 0.3380268 | | | | 0.5457352 | |
| PC 15:0_18:2 | 1.4274936 | 0.8699278 | 1.1976567 | 1.586999 | 0.7767495 | 0.7107978 | 1.0799792 | 1.2592762 | 1.4148072 | 0.8839356 |
| PC 16:0_16:0 | 43.167206 | 31.562768 | 39.929913 | 45.595307 | 29.693213 | 23.943789 | 36.312426 | 36.799688 | 37.97764 | 31.864149 |
| PC 16:0_16:1 | 3.4740522 | 3.8748756 | 3.0128359 | 3.0755207 | 2.325096 | 2.2843595 | 2.1531196 | 2.5664951 | 3.5678376 | 3.5852224 |
| PC 16:0_18:0 | 48.844967 | 38.189124 | 54.668454 | 55.072242 | 40.462256 | 38.582564 | 38.678006 | 45.078671 | 41.516428 | 36.471896 |
| PC 16:0_18:1 | 161.93172 | 180.4279 | 152.5388 | 127.75653 | 97.66697 | 113.86026 | 109.12593 | 125.32467 | 147.67238 | 163.45515 |
| PC 16:0_18:2 | 817.4712 | 468.12402 | 699.93785 | 861.73325 | 485.27417 | 384.75664 | 664.87907 | 708.03156 | 775.86767 | 613.8447 |
| PC 16:0_18:3 | 3.5342902 | 2.6688289 | 3.9679569 | 3.3311706 | 2.513969 | 2.5774221 | 3.512719 | 3.6872169 | 4.3187708 | 3.3406382 |
| PC 16:0_20:1 | 4.6441487 | 1.8988296 | 3.3212738 | 5.3127415 | 3.6342264 | 1.186076 | 2.5740663 | 2.9585783 | 3.5773144 | 3.0718563 |
| PC 16:0_20:2 | 4.1199859 | 4.9482236 | 4.6628536 | 3.4443442 | 2.8413294 | 2.4554849 | 2.9481099 | 3.9175778 | 3.3574104 | 4.4358338 |
| PC 16:0_20:3 | 54.987463 | 54.906432 | 48.204426 | 46.503377 | 32.91804 | 34.480044 | 44.267317 | 59.149976 | 49.107715 | 46.05237 |
| PC 16:0_20:4 | 187.21102 | 140.93722 | 168.66929 | 182.77861 | 138.60484 | 120.71013 | 164.12396 | 194.59614 | 183.55143 | 188.33711 |
| PC 16:0_20:5 | 25.280392 | 27.57052 | 23.478688 | 19.025419 | 19.856503 | 17.061245 | 33.062582 | 27.485162 | 41.352122 | 22.49174 |
| PC 16:0_22:1 | | | | | | | | | | |
| PC 16:0_22:2 | | | | | | | | 0.4072122 | 0.3465007 | |
| PC 16:0_22:4 | 1.9748346 | 2.1995301 | 1.9494395 | | | 1.184073 | 1.4417926 | 1.9868909 | 1.5478358 | 2.6533455 |
| PC 16:0_22:5 | 22.080952 | 14.49043 | 21.093556 | 16.154777 | 7.8923238 | 12.424314 | 11.86118 | 14.394119 | 18.86702 | 14.840852 |
| PC 16:0_22:6 | 299.1005 | 213.35375 | 213.45982 | 204.03926 | 122.5536 | 131.22938 | 212.46576 | 279.63728 | 290.34119 | 225.10886 |
| PC 16:1_17:0 | | | | | | | | | | |
| PC 16:1_18:0 | 0.528965 | 0.8642824 | 0.4946834 | 0.6483476 | 0.5259313 | 0.4187057 | 0.3601659 | | 0.5473643 | 0.6163077 |
| PC 16:1_18:1 | 4.0820855 | 6.5929836 | 4.0611002 | 2.7940755 | 2.2093952 | 3.4389896 | 2.3829433 | 3.0843683 | 4.0693001 | 5.2497968 |
| PC 16:1_18:2 | 10.385903 | 8.1106863 | 9.8104437 | 10.227015 | 6.4156956 | 5.8865438 | 8.1708203 | 9.1438908 | 10.669473 | 9.156314 |
| PC 16:1_20:0 | | | | 0.4448345 | | | | | 0.2983891 | |
| PC 17:0_18:1 | 1.083673 | 1.1026991 | 0.8940908 | 0.8963162 | 0.6005345 | 0.7603994 | 0.9335859 | 1.0300045 | 1.1836269 | 0.8810716 |
| PC 17:0_18:2 | 7.002754 | 3.9542138 | 5.9944021 | 6.8601169 | 4.7674382 | 3.2772304 | 5.2317996 | 5.6741749 | 6.4713126 | 4.0881907 |
| PC 17:0_20:3 | 1.7577833 | 0.8891505 | | | | 1.0357808 | | 1.2953389 | | |
| PC 17:0_20:4 | 6.9983955 | 4.2628553 | 4.6100506 | 5.3522357 | 5.325811 | 3.0600442 | 5.5410661 | 6.0810918 | 5.6405505 | 3.9582258 |
| PC 17:0_22:6 | 7.1090841 | 3.2525098 | | 6.7053171 | | | | 5.5773684 | 6.1818122 | |
| PC 18:0_18:0 | 0.7479356 | 0.9692622 | 0.6955262 | 0.7333491 | 0.5605608 | 0.9067738 | 0.6289907 | 0.6836789 | 0.5360707 | 0.7335761 |
| PC 18:0_18:1 | 23.654044 | 23.44038 | 24.915802 | 23.443111 | 17.6499 | 21.743457 | 15.520922 | 20.15638 | 22.562086 | 22.090758 |
| PC 18:0_18:2 | 372.14137 | 225.4161 | 373.47486 | 397.37334 | 267.93304 | 242.45421 | 254.62414 | 326.09749 | 334.29436 | 264.15808 |
| PC 18:0_18:3 | 0.9792107 | 1.0155499 | 1.3515215 | 1.165032 | 0.8554777 | 0.942482 | | 1.0621541 | 1.1305457 | 1.1518707 |
| PC 18:0_20:0 | 9.0535902 | 3.1912543 | 7.6210682 | 9.7109339 | 5.6861071 | 4.3941474 | 5.0810134 | 6.1821338 | 5.3566862 | 4.2516046 |
| PC 18:0_20:1 | 1.1302071 | 0.4812811 | 0.8853684 | 1.3502431 | 1.4912075 | 0.4658764 | 0.601095 | 0.5994531 | 0.979544 | 0.9713384 |
| PC 18:0_20:2 | 1.2520299 | 1.4915213 | 1.6657764 | 1.0886702 | 0.7737213 | 1.1861746 | 0.9169151 | 1.3091161 | 0.951995 | 1.2659957 |
| PC 18:0_20:3 | 22.650418 | 22.459644 | 22.292757 | 21.365768 | 17.221063 | 20.745188 | 16.219054 | 24.4659 | 17.680232 | 19.190193 |
| PC 18:0_20:4 | 102.0124 | 72.091813 | 93.137015 | 108.86861 | 93.443215 | 101.57106 | 77.979595 | 110.61404 | 91.927789 | 92.928944 |
| PC 18:0_20:5 | 14.223972 | 14.482841 | 12.254711 | 12.700982 | 12.584212 | 13.33748 | 13.392539 | 15.050415 | 17.360952 | 10.624253 |
| PC 18:0_22:4 | | | | | | 0.9616638 | | | | |
| PC 18:0_22:5 | 5.5270933 | 4.5474763 | 6.1754039 | 5.6205162 | 3.6307815 | 5.8497701 | 2.7238081 | 4.4382261 | 6.2793282 | 4.3391063 |
| PC 18:0_22:6 | 87.459867 | 53.820475 | 53.577325 | 57.76706 | 41.580656 | 57.891791 | 61.656605 | 79.583226 | 77.939789 | 59.671497 |
| PC 18:1_18:1 | 5.6857443 | 9.9056319 | 5.5313309 | 6.7704455 | 5.2418374 | 5.3879231 | 3.2765029 | 3.9783282 | 4.8118942 | 7.3035512 |
| PC 18:1_18:2 | 75.627277 | 66.224007 | 71.047656 | 89.522914 | 62.612301 | 46.75846 | 44.394528 | 54.620434 | 63.339915 | 68.393685 |
| PC 18:1_18:3 | | 0.7987698 | | | | | | | | |
| PC 18:1_20:0 | 3.2542191 | 1.6956043 | 2.7111211 | 3.906608 | 2.1882358 | 1.5225333 | 1.8114429 | 2.4522332 | 2.7347171 | 2.1387032 |
| PC 18:1_20:1 | 0.8280098 | 0.4875746 | 0.3898633 | 0.6147217 | 0.4980139 | | 0.3386501 | 0.3961294 | 0.3701125 | 0.4304619 |
| PC 18:1_20:2 | 0.574024 | 0.974024 | 0.4753937 | | | | | 0.3586467 | | |
| PC 18:1_20:3 | 3.6273863 | 8.2923432 | 3.4604682 | 3.3093855 | 3.6874566 | 3.6534161 | 2.6529449 | 3.4647204 | 3.4960657 | 5.2059736 |
| PC 18:1_20:4 | 18.242911 | 20.476811 | 14.100282 | 14.825372 | 16.41188 | 15.855229 | 12.378622 | 14.009235 | 14.2733 | 19.745055 |
| PC 18:1_20:5 | 2.3076368 | 4.4898715 | 2.545595 | | 3.0041419 | 2.0604305 | 3.2604651 | | 3.5924889 | 3.6573014 |
| PC 18:1_22:5 | | | | | | | | | | 1.5168728 |
| PC 18:1_22:6 | 16.988382 | 27.749422 | 11.491965 | 15.314126 | 9.8042012 | 13.230019 | 12.266358 | 18.852251 | 16.997184 | 21.234469 |
| PC 18:2_18:2 | 15.427156 | 9.5132622 | 17.561162 | 22.781532 | 11.209402 | 8.0954037 | 12.820602 | 13.270134 | 16.007533 | 10.569089 |
| PC 18:2_18:3 | | | 1.0152365 | 1.2659148 | | | 1.6459286 | 1.1531024 | 1.484065 | |
| PC 18:2_20:0 | 13.560004 | 4.6144591 | 12.815477 | 15.621142 | 7.2747336 | 4.9900188 | 9.8449365 | 12.721338 | 12.404647 | 6.756266 |
| PC 18:2_20:1 | 2.2026961 | 1.4672662 | 2.4188516 | 2.7374401 | 1.3479191 | 0.9831883 | 1.3994368 | 1.7201715 | 1.8279443 | 1.1849291 |
| PC 18:2_20:2 | | 0.6730044 | | 0.9414668 | 0.7053637 | 0.5120823 | 0.7415748 | 0.8939289 | 0.9432062 | 0.649322 |
| PC 18:2_20:3 | 1.5370104 | 1.6650689 | 1.6854773 | 1.3340607 | 1.2182682 | 1.2756396 | 1.3521941 | 1.8780783 | 1.9423606 | 1.1478792 |
| PC 18:2_20:4 | 6.6944154 | 4.6085332 | 7.4458475 | 6.8119782 | 4.9196566 | 4.3635804 | 5.7077954 | 7.3357375 | 7.4279285 | 4.6514194 |
| PC 18:2_20:5 | | | | | | | | | 2.6479289 | |
| PC 18:2_22:6 | | | | | | | | | 5.794243 | |
| PC 20:0_20:3 | | | | | | | | 0.9967018 | | |
| PC 20:0_20:4 | 3.8482551 | 1.5391541 | 3.0217981 | 4.3658937 | 2.292245 | 1.7055152 | 3.5600927 | 4.1702616 | 3.6919398 | 2.6802525 |
| PC 20:0_22:6 | | | | | | | | | | |
| PE 16:0_18:1 | | | | 0.9949391 | 1.041372 | 0.696802 | | | 1.218149 | |
| PE 16:0_18:2 | | 4.0718708 | 5.8585092 | 8.0354332 | 3.9030563 | 3.3112781 | 4.9054198 | 4.5972006 | | |
| PE 16:0_20:4 | | | | 4.8195863 | | 3.7747217 | | | | |
| PE 18:0_18:0 | | 1.0559308 | 0.6475951 | 0.5945533 | 0.5433193 | 0.6348792 | 0.6238269 | 0.8203832 | 1.1903685 | 1.2561211 |
| PE 18:0_18:1 | | 110.30449 | 54.239152 | 26.125876 | 27.636462 | 38.667659 | 36.554039 | 42.94824 | 72.205601 | 81.045751 |
| PE 18:0_18:2 | | 287.55866 | 241.73055 | 209.26901 | 132.1946 | 126.95901 | 208.64062 | 212.42933 | | |
| PE 18:0_20:1 | | 2.0529367 | 2.1277013 | 2.2075721 | 2.0437195 | 1.027735 | 1.9437386 | 1.8797901 | 3.2347057 | 3.2741479 |
| PE 18:0_20:2 | | 7.2030632 | 5.2783317 | 4.3110605 | 3.0016255 | 2.2233026 | 3.5592444 | 4.4826435 | | |
| PE 18:0_20:4 | | | | 49.62598 | | 44.852753 | | | | |
| PE 18:0_22:2 | | | | 0.7178923 | | | | | | |
| PE 18:0_22:4 | | | | 1.4407335 | | 1.0135854 | | | | |
| PE 18:1_18:1 | | 1.8016724 | 0.8534802 | 0.6121147 | 0.5554721 | 0.5547941 | 0.4422947 | 0.6352862 | 0.951975 | 1.3757758 |
| PE 18:1_18:2 | | 6.4909376 | 5.0754322 | 5.9381083 | 3.672898 | 3.1061143 | 3.355848 | 3.3657283 | | |
| PE 18:1_20:4 | | | | 3.2653964 | | 2.5007644 | | | | |
| PE 18:1_22:0 | | 6.9273345 | 2.5739804 | 2.5523807 | 2.7017753 | 2.1317785 | 1.7497919 | 2.9556958 | 4.3146408 | 3.8666015 |
| PE 18:2_18:2 | | | | 0.7231908 | | | | | | |
| PE O-16:0/18:1 | | | | | | | | 0.2682919 | | |
| PE O-16:0/18:2 | | | 1.1322022 | 1.979693 | 1.0104494 | | 1.1123884 | 1.006937 | | |
| PE O-16:0/20:4 | | | | 1.2990187 | | | | | | |
| PE O-18:0/16:0 | | 1.4045735 | 1.0067857 | 0.8181271 | 0.9745635 | 0.7513622 | 0.7866817 | 0.9985967 | 1.5046513 | 1.2084537 |
| PE O-18:0/18:1 | | 1.5966002 | 1.1639424 | 0.9519813 | 0.8295993 | 0.5877857 | 1.0846036 | 1.1713362 | 1.8784337 | 1.2539744 |
| PE O-18:0/18:2 | | 4.9357352 | 5.3828136 | 6.2532496 | 3.7666863 | 2.9382706 | 4.4954246 | 3.897373 | | |
| PE O-18:0/20:4 | | | | 4.5345952 | | 2.5131171 | | | | |
| PE P-16:0/16:0 | | | | | | | | | | |
| PE P-16:0/18:1 | | | | | 0.4648096 | | | | | |
| PE P-16:0/18:2 | | 1.7357857 | 1.1228675 | 1.6315885 | 1.2007808 | 1.0508686 | 1.5141801 | 1.3109976 | | |
| PE P-16:0/20:4 | | | | 2.7395697 | | 1.4728988 | | | | |
| PE P-18:0/16:0 | | | 1.1932711 | 0.8183499 | 0.8701454 | | 0.943309 | 0.848404 | 1.6645531 | 1.2487854 |
| PE P-18:0/18:1 | | | | 0.5606779 | | 0.5503419 | 0.6512092 | 0.7853492 | 1.5567653 | |
| PE P-18:0/18:2 | | 3.6272203 | 2.4450253 | 2.9784499 | 2.3383754 | 2.1379844 | 2.4702643 | 3.1503254 | | |
| PE P-18:0/20:4 | | | | 4.8734093 | | 3.7174043 | | | | |
| PE P-18:1/18:1 | | | | | | | | | | |
| PE P-18:1/18:2 | | | | 0.4983263 | | | | | | |
| PE P-18:1/20:4 | | | | | | | | | | |
| PG 16:0_18:1 | 0.1761487 | | 0.1494805 | 0.2366119 | 0.1640523 | | 0.130722 | 0.1368327 | 0.1635018 | 0.1959548 |
| PG 16:0_18:2 | 0.2359194 | 0.1061032 | 0.2038487 | 0.2517634 | 0.1466308 | 0.0711554 | 0.1110195 | 0.1670975 | 0.2095305 | 0.1628394 |
| PG 16:1_18:1 | 0.1576093 | 0.1442568 | 0.1457008 | 0.1572669 | 0.1310393 | 0.1077339 | 0.1070474 | 0.1187739 | 0.1739101 | 0.1293814 |
| PG 16:1_18:2 | 0.5542768 | 0.3689965 | 0.6042037 | 1.1875482 | 0.4447837 | 0.3712546 | 0.6053019 | 0.6545529 | 0.7040491 | 0.4488559 |
| PG 17:0_20:0 | 0.467738 | 0.5670793 | 0.6132529 | 0.5347422 | 0.4900898 | 0.442292 | 0.4360757 | 0.4849129 | 0.647665 | 0.5460415 |
| PG 17:0_22:1 | | | | | | | | | | |
| PG 18:0_18:0 | | | | | | | | | | |
| PG 18:0_18:1 | 0.2235385 | 0.276952 | 0.1286475 | 0.1381606 | 0.1071337 | | 0.1191739 | 0.1268965 | 0.2253356 | 0.3527854 |
| PG 18:0_18:2 | 0.9696397 | 0.7280405 | 0.6252484 | 0.5438926 | 0.3050322 | 0.2736724 | 0.5768867 | 0.557455 | 1.0463973 | 0.8288163 |
| PG 18:0_20:0 | 0.4145912 | 0.6195734 | 0.3515381 | 0.3098329 | | 0.2719803 | | 0.4602176 | 0.6075589 | 0.3945654 |
| PG 18:0_20:4 | 1.7485047 | 1.4473444 | 0.6714176 | | 0.4459039 | 0.3674997 | 0.4372762 | 0.8752752 | 1.3063283 | 1.8506688 |
| PG 18:0_22:5 | | 0.2208722 | | | | | | | | |
| PG 18:0_22:6 | | 0.9846528 | | | | 0.330373 | | 0.7340049 | 1.2275552 | 1.0766252 |
| PG 18:1_18:1 | 0.0619913 | 0.0729047 | 0.0777824 | 0.089682 | 0.071527 | 0.0622067 | 0.0481957 | 0.0557222 | 0.0828173 | 0.0784482 |
| PG 18:1_18:2 | 0.9168816 | 0.6554303 | 1.1012863 | 1.4213901 | 0.6340742 | 0.5626757 | 0.7372837 | 0.8062754 | 1.0788656 | 0.8935416 |
| PG 18:1_20:0 | 0.1908533 | 0.1539914 | 0.1811683 | 0.1647266 | 0.1180515 | | 0.1176206 | 0.0731534 | 0.2281693 | 0.1848803 |
| PG 18:1_20:3 | | | | 0.1786161 | | 0.1310949 | | 0.1548069 | | 0.1863053 |
| PG 18:1_20:4 | 1.4191404 | 0.9096994 | 1.5170754 | 1.7203575 | 0.9787871 | 1.1490735 | 1.1429036 | 1.327495 | 1.5058 | 1.2428645 |
| PG 18:1_22:6 | | | | | | | | | | 0.4792281 |
| PG 18:2_18:2 | 0.0922467 | 0.1022836 | 0.1111314 | 0.1537448 | 0.0882444 | 0.0667472 | 0.0669247 | 0.0763024 | 0.1251298 | 0.1193355 |
| PG 18:2_20:0 | 1.7006949 | 1.222014 | 1.1984745 | 1.0055525 | 0.4190705 | 0.5096117 | 0.631791 | 0.7100082 | 1.7977264 | 1.2039079 |
| PG 18:2_20:4 | | | | | | | | 0.3763649 | 0.4605017 | 0.5085905 |
| PG 20:0_20:3 | | 0.2650942 | | | | | | 0.1074838 | 0.259894 | |
| PG 20:0_20:4 | 4.7759966 | 2.7187217 | 1.8225779 | 1.7312005 | 1.3111222 | 1.3275227 | 1.6878148 | 2.1852791 | 4.0748784 | 4.2114128 |
| PG 20:0_20:5 | | | | | | | | | 0.4921385 | |
| PG 20:0_22:6 | 0.8167606 | 0.4842224 | | | | | | | 0.8108987 | |
| PI 14:0_18:1 | 1.1309429 | 1.0915381 | 0.7710926 | 0.7941733 | 0.4876113 | 0.6780326 | 0.7251083 | 0.9313474 | 1.1691011 | 1.1367509 |
| PI 14:0_18:2 | 0.4426838 | 0.5685919 | | | | | | | 0.3597836 | |
| PI 15:0_18:1 | | | | | | | | | | |
| PI 15:0_20:1 | | | | | | | | | | |
| PI 15:0_20:2 | 0.4975283 | | 0.467021 | 0.5546574 | 0.3623424 | | 0.3613672 | | 0.6070998 | |
| PI 15:0_22:0 | 0.8757225 | 0.9009402 | 0.9355593 | 0.7818073 | 0.6272747 | 0.8423484 | 0.9052533 | 0.7469465 | 0.9197045 | 0.819529 |
| PI 15:0_22:1 | 0.4444977 | | | 0.3958806 | 0.5060992 | | 0.2730592 | | 0.4966012 | 0.4060676 |
| PI 15:0_22:2 | 0.7825777 | | 0.6445555 | 0.8750171 | 0.6547906 | | | | 1.0045958 | |
| PI 15:0_22:4 | 1.0217005 | 0.8280067 | 1.0498038 | 0.635493 | 0.7637511 | 0.4939061 | 0.6369775 | 1.0471492 | 1.2011856 | 0.7160572 |
| PI 15:0_22:6 | | | | | | | | | | |
| PI 16:0_16:0 | 0.1362584 | | | 0.2039762 | 0.1264624 | | 0.1252819 | | 0.1321386 | 0.1766162 |
| PI 16:0_18:0 | | | | | | | | | | 0.3064584 |
| PI 16:0_18:1 | 1.9163619 | 0.9935966 | 2.0677261 | 2.6755612 | 1.4211594 | 1.0525629 | 1.2687231 | 1.6077157 | 1.8293261 | 1.4298134 |
| PI 16:0_18:2 | 8.6646599 | 3.9655445 | 9.5059483 | 10.977548 | 6.0424375 | 3.5245792 | 7.4588597 | 8.8346425 | 10.115388 | 6.5664031 |
| PI 16:0_20:2 | | 0.3035381 | | | | | | | | 0.4828976 |
| PI 16:0_20:3 | 2.9692179 | 2.2060199 | 2.8983237 | 2.4843982 | 1.9223721 | 1.8314661 | 2.100303 | 2.3903277 | 2.8757168 | 2.411047 |
| PI 16:0_20:4 | 10.827474 | 8.7567714 | 12.287867 | 9.3359156 | 6.4944626 | 6.8196213 | 8.1173718 | 11.194882 | 12.171508 | 10.549121 |
| PI 16:0_20:5 | | | | | | | | | | |
| PI 16:0_22:5 | | | 0.9127854 | | | | | | | |
| PI 16:0_22:6 | | | | | | | | | | |
| PI 17:0_18:1 | 0.423407 | | 0.2697544 | 0.4230512 | 0.4269942 | 0.1551736 | | | 0.3782631 | 0.3171732 |
| PI 17:0_18:2 | 0.7823669 | 0.2888283 | 0.6493472 | 0.6997495 | 0.5504162 | 0.3323178 | 0.4604253 | 0.5655262 | 0.6949235 | 0.3834436 |
| PI 17:0_20:0 | 1.0865609 | 1.4984063 | 1.2571197 | 1.0781931 | 1.2171117 | 1.3463188 | 1.1584439 | 1.8022907 | 1.3920275 | 1.3269175 |
| PI 17:0_20:1 | 0.3750268 | | | | 0.2933161 | 0.1956449 | | | 0.4012968 | |
| PI 17:0_20:3 | | | | | | | 0.2854042 | 0.2813425 | 0.5326054 | |
| PI 17:0_20:4 | 1.5902868 | 1.1644723 | 1.3157149 | 1.363299 | 1.1029578 | 1.1191993 | 1.2752089 | 1.3813915 | 1.9729155 | 1.2549487 |
| PI 17:0_22:1 | 1.1014702 | 0.9024178 | 1.145905 | 1.1904034 | 1.0436124 | 1.2207169 | 1.0004406 | 1.1470875 | 1.0231919 | 1.5017347 |
| PI 17:0_22:2 | | | | | | | | | | |
| PI 18:0_18:0 | 0.1385094 | | | | | | | | 0.109578 | 0.0979183 |
| PI 18:0_18:1 | 1.9641593 | 0.9463752 | 1.8329866 | 2.5446068 | 1.4901186 | 1.0588186 | 1.2554132 | 1.4964367 | 2.1211637 | 1.3147461 |
| PI 18:0_18:2 | 13.369677 | 6.1358874 | 12.709085 | 15.660284 | 8.6112529 | 6.6165602 | 10.882856 | 12.034641 | 14.812032 | 8.8970445 |
| PI 18:0_20:0 | 1.0014582 | | 0.6871868 | 1.1010986 | 0.7142023 | 0.3729661 | 0.6692562 | 0.5440726 | 0.9131284 | 0.4644693 |
| PI 18:0_20:2 | 0.6723991 | 0.5362494 | | 0.5442348 | | | 0.4279576 | 0.3827594 | 0.5133957 | 0.4318208 |
| PI 18:0_20:3 | 8.6202954 | 9.5533211 | 7.2000584 | 7.6197606 | 5.4866262 | 6.9179271 | 7.7642795 | 8.9389879 | 8.0206057 | 6.9323485 |
| PI 18:0_20:4 | 110.38479 | 79.911595 | 88.257844 | 75.488279 | 74.083092 | 82.637993 | 85.875723 | 134.35041 | 137.14639 | 110.76169 |
| PI 18:0_20:5 | 1.4242024 | 1.2266055 | 0.9744058 | | | 1.0704381 | 1.2471933 | 1.5249013 | 2.0175813 | 1.5882802 |
| PI 18:0_22:4 | | | | | | 0.4533228 | | | | |
| PI 18:0_22:5 | 1.388912 | 1.2738397 | | | | 0.7760433 | 1.1865067 | 1.1033582 | 1.3689048 | |
| PI 18:0_22:6 | 1.3245604 | 1.1558342 | 1.40753 | | | 0.7632066 | 1.6115088 | 1.5940028 | 2.0219993 | |
| PI 18:1_18:1 | 0.5849974 | 0.4021422 | 0.5044583 | 0.7965933 | 0.4898565 | 0.3488562 | 0.4291581 | 0.4872418 | 0.6648898 | 0.4119485 |
| PI 18:1_18:2 | 2.4464823 | 1.2488564 | 2.0073137 | 2.9155707 | 1.4360818 | 1.0035188 | 1.6723231 | 1.9771787 | 2.740522 | 1.4559597 |
| PI 18:1_20:0 | | | | 0.4387128 | | | 0.3064542 | | | |
| PI 18:1_20:3 | | 0.5431935 | | 0.4351619 | | | | | 0.4976604 | |
| PI 18:1_20:4 | 2.0595559 | 2.8078943 | 2.2759392 | 2.3496857 | 2.3277623 | 2.0091264 | 2.6220519 | 3.6086875 | 3.2163989 | 4.3486677 |
| PI 18:2_18:2 | 0.2589539 | | 0.2313626 | 0.3950855 | | | 0.239406 | 0.254208 | 0.3443219 | |
| PI 18:2_20:0 | | | | | | | 0.2855301 | 0.3768582 | | |
| PI 20:0_20:4 | 0.9007487 | | | | | | 0.8634438 | 1.4850009 | 1.2173638 | |
| PS 16:0_18:2 | | | | | | | | | | |
| PS 16:0_22:6 | | | | | | | | | | 3.5044173 |
| PS 16:1_18:1 | | | | | | | | | 1.8312588 | 1.6652522 |
| PS 16:1_18:2 | 3.8032818 | 2.7214689 | 2.3847824 | 2.261621 | 1.2009138 | | 1.4488058 | 1.8248114 | 4.5254436 | 2.4681468 |
| PS 17:0_20:0 | 1.0023878 | 1.0558551 | 1.355176 | 1.1191074 | 1.1292857 | 0.9124057 | 1.2307517 | 1.3318971 | 1.0502747 | 0.8391593 |
| PS 17:0_20:3 | 3.0189494 | 1.5220105 | 1.0212647 | 1.7820961 | 1.0044666 | 0.8729661 | 1.5059751 | 0.9299645 | 2.0869526 | 1.7756404 |
| PS 17:0_22:1 | | | 1.3649461 | 1.2272117 | | | | | | |
| PS 18:0_18:0 | 0.3933844 | 0.3433014 | 0.4019972 | 0.1766817 | | | 0.2673609 | 0.230922 | 0.5208584 | 0.3787603 |
| PS 18:0_18:1 | 2.1176971 | 2.3519556 | | 1.0921704 | | | | | 2.415818 | 1.808199 |
| PS 18:0_18:2 | 6.226063 | 6.8683771 | 2.2290277 | 2.1696814 | 1.0573213 | | 1.2034835 | 2.3639769 | 7.3893198 | 4.5522899 |
| PS 18:0_20:0 | 1.8091467 | 1.2680543 | 1.2468963 | 0.9430938 | | 0.5985349 | 1.0597902 | 1.137507 | 2.6875741 | 0.8734047 |
| PS 18:0_20:4 | | | | | | | | | | 5.1144493 |
| PS 18:0_22:5 | | | | | | | | | | 3.331588 |
| PS 18:0_22:6 | | | | | | 3.5231176 | | | | 9.9823418 |
| PS 18:1_18:1 | 1.0539394 | 0.8636383 | 0.579814 | 0.6745249 | | | | | 1.2201314 | 1.2025029 |
| PS 18:1_18:2 | 5.3129279 | 6.2237857 | 4.128748 | 4.1499537 | 1.8926088 | 1.6272505 | 2.5287925 | 3.4679578 | 9.527237 | 5.588753 |
| PS 18:1_20:0 | 2.8588336 | 3.8470748 | 1.793762 | 1.7387756 | | 1.1236628 | | 1.7622882 | 3.9449238 | 3.0400135 |
| PS 18:1_20:4 | | | | | | 3.3775909 | | | | 10.415454 |
| PS 18:1_22:5 | | | | | | | | | | 2.6859036 |
| PS 18:1_22:6 | | | | | | 3.8564014 | | | | 10.0995 |
| PS 18:2_18:2 | | | 0.6237426 | 0.6095888 | | | | | 0.9584827 | 1.0281513 |
| PS 18:2_20:0 | 7.0368964 | 8.0118501 | 3.6630166 | 2.995906 | 1.5836721 | 1.8109518 | 1.8177794 | 2.5542022 | 9.8274707 | 7.2142447 |
| PS 20:0_20:4 | | | | | | 3.2790714 | | | | 8.4010022 |
| PS 20:0_22:6 | | | | | | | | | | 7.4907486 |
| SM d18:1/14:0 | 1.3645945 | 0.8189513 | 1.097563 | 1.1369479 | 1.2414398 | 0.8727753 | 0.953672 | 1.0804701 | 1.4073389 | 1.2813371 |
| SM d18:1/16:0 | 43.135907 | 30.866814 | 33.421169 | 33.203014 | 36.249114 | 31.771178 | 26.019429 | 29.876879 | 37.932422 | 33.10269 |
| SM d18:1/18:0 | 4.1364207 | 3.7907079 | 3.6279233 | 3.5692287 | 4.9993558 | 4.5783296 | 2.6315035 | 3.0475759 | 3.9469829 | 3.909665 |
| SM d18:1/18:1 | 1.8290578 | 1.4191548 | 1.6242797 | 1.4645048 | 1.7789071 | 1.4197729 | 1.1906245 | 1.4524335 | 1.68684 | 1.7426575 |
| SM d18:1/20:0 | 44.824624 | 32.397826 | 38.915101 | 46.389793 | 27.115741 | 25.951286 | 36.054171 | 42.052591 | 46.786899 | 36.999478 |
| SM d18:1/20:1 | 2.2859626 | 1.2780207 | 1.9228553 | 2.1174295 | 1.8103136 | 1.368468 | 1.7553974 | 1.9623414 | 2.4252661 | 2.0316282 |
| SM d18:1/22:0 | 55.361639 | 32.709295 | 49.739521 | 53.537556 | 33.52259 | 35.060193 | 46.140433 | 57.305095 | 56.76286 | 36.271248 |
| SM d18:1/22:1 | 15.796273 | 10.65971 | 13.832997 | 14.071083 | 8.862012 | 8.2579307 | 12.477629 | 14.81595 | 16.405004 | 11.514895 |
| SM d18:1/24:0 | 15.106196 | 8.6367449 | 11.898698 | 13.177158 | 9.1830666 | 8.8481208 | 11.434286 | 13.528678 | 14.028875 | 9.532497 |
| SM d18:1/24:1 | 24.682396 | 30.988794 | 21.548572 | 21.54478 | 27.019513 | 32.097821 | 17.310234 | 22.686671 | 21.251311 | 30.43021 |
| SM d18:1/26:0 | 2.8693271 | 1.2019357 | 2.2093295 | 2.758016 | 2.6541148 | 1.843797 | 2.0813071 | 2.5481655 | 2.1828541 | 2.3634116 |
| SM d18:1/26:1 | 4.064774 | 1.8110092 | 3.8674829 | 3.9754689 | 3.1862104 | 2.5928136 | 3.1275628 | 3.593136 | 3.6803088 | 3.5145305 |
| TG 36:0-FA12:0 | | | | | | 0.0421423 | | | | |
| TG 38:0-FA12:0 | | | | | | | | | | |
| TG 40:0-FA12:0 | | | | | | | | | | |
| TG 40:0-FA16:0 | 0.1174668 | | | 0.1005944 | 0.140555 | | | 0.0611401 | 0.0856821 | 0.1294091 |
| TG 42:0-FA16:0 | 0.1734713 | | 0.1340947 | 0.1816362 | 0.1728721 | 0.0704887 | 0.1102615 | 0.0655638 | 0.1092053 | 0.1768161 |
| TG 42:1-FA16:0 | 0.3532192 | 0.0663032 | 0.2111702 | 0.2865689 | 0.3470531 | 0.1262844 | 0.1941843 | 0.1295606 | 0.2865506 | 0.356425 |
| TG 42:1-FA16:1 | | | | | | | | | | |
| TG 42:1-FA18:1 | 0.1836688 | 0.0693574 | 0.1330502 | 0.1387798 | 0.1614376 | 0.0554884 | 0.0882908 | 0.050802 | 0.1036931 | 0.2144738 |
| TG 42:2-FA18:2 | 0.2563208 | 0.0524743 | 0.1220686 | 0.2384598 | 0.2252579 | 0.1070841 | 0.1908233 | 0.1203637 | 0.2072046 | 0.2044694 |
| TG 44:0-FA12:0 | | | | | | | | | 0.0747944 | |
| TG 44:0-FA14:0 | | | | | | | | | 0.0853352 | |
| TG 44:0-FA16:0 | 0.1489813 | 0.0768734 | 0.1473583 | 0.1177972 | 0.1552815 | 0.076209 | 0.1227034 | 0.0822556 | 0.1614901 | 0.1287157 |
| TG 44:0-FA18:0 | 0.0875478 | | | | 0.0728069 | | | | | |
| TG 44:1-FA14:0 | | | | | | | | | | |
| TG 44:1-FA16:0 | 0.5642972 | 0.1504893 | 0.2608048 | 0.491767 | 0.6178757 | 0.2045989 | 0.2318351 | 0.1952998 | 0.3542057 | 0.6168534 |
| TG 44:1-FA18:1 | 0.3308591 | 0.0917967 | 0.2147673 | 0.2981831 | 0.3798459 | 0.1025641 | 0.1391209 | 0.0956362 | 0.1723165 | 0.3469197 |
| TG 44:2-FA16:0 | 0.8563132 | 0.1243121 | 0.3525854 | 0.6555889 | 0.7058721 | 0.2790517 | 0.4624744 | 0.2997945 | 0.4714965 | 0.6244296 |
| TG 44:2-FA16:1 | 0.1018693 | | | | 0.1178661 | | | | | 0.0784382 |
| TG 44:2-FA18:1 | 0.2038794 | 0.0961852 | 0.1099243 | 0.1619264 | 0.2040979 | 0.0783651 | 0.0946035 | 0.0651552 | 0.145064 | 0.2729373 |
| TG 44:2-FA18:2 | 0.4265327 | | 0.1924986 | 0.4194344 | 0.3621141 | 0.1250179 | 0.2320619 | 0.1384683 | 0.2195585 | 0.2849755 |
| TG 44:3-FA18:2 | 0.2804639 | 0.0686336 | 0.1189619 | 0.2320123 | 0.2065347 | 0.1187343 | 0.1466901 | 0.0990095 | 0.1571184 | 0.2183311 |
| TG 45:0-FA16:0 | 0.2336689 | | 0.1745118 | 0.1495975 | 0.2460511 | 0.0860783 | 0.1506797 | 0.1215812 | 0.1261391 | 0.1937146 |
| TG 45:1-FA16:0 | 0.636318 | 0.1221972 | 0.2972674 | 0.5330223 | 0.6123212 | 0.1952572 | 0.2783535 | 0.1895693 | 0.3594265 | 0.5010461 |
| TG 45:1-FA18:1 | 0.2819835 | 0.0649913 | 0.1605244 | 0.2210928 | 0.31219 | 0.0871903 | 0.09411 | | 0.1441018 | 0.3018725 |
| TG 46:0-FA12:0 | | | | | | | | | 0.0351142 | 0.0192365 |
| TG 46:0-FA14:0 | 0.1361691 | 0.1981686 | 0.3308466 | 0.1623113 | 0.1804284 | 0.1105493 | 0.2105548 | 0.1470072 | 0.3544217 | 0.1683647 |
| TG 46:0-FA16:0 | 0.3464727 | 0.3951392 | 0.7858345 | 0.3160399 | 0.4559042 | 0.2642235 | 0.4153124 | 0.318781 | 0.7146232 | 0.4216561 |
| TG 46:1-FA12:0 | 0.3414086 | 0.1769308 | 0.2452723 | 0.2484961 | 0.2639001 | 0.1532531 | 0.1930552 | 0.141442 | 0.2565052 | 0.3667913 |
| TG 46:1-FA14:0 | 0.0968551 | 0.2151986 | 0.2267973 | 0.0891519 | 0.1238777 | 0.1444096 | 0.1116687 | 0.0798513 | 0.2243497 | 0.2398473 |
| TG 46:1-FA14:1 | | 0.03789 | | 0.1006868 | 0.083904 | | 0.0660911 | | 0.0980084 | 0.0883948 |
| TG 46:1-FA16:0 | 0.5195409 | 0.2509648 | 0.3454351 | 0.4660782 | 0.6149436 | 0.2719879 | 0.3451766 | 0.2016617 | 0.4949375 | 0.4337654 |
| TG 46:1-FA16:1 | | 0.1552724 | 0.1944506 | | 0.105703 | 0.0758109 | 0.0913129 | 0.0613195 | 0.2212208 | 0.1419276 |
| TG 46:1-FA18:0 | 0.0844099 | | 0.0603395 | 0.0914052 | 0.1040349 | 0.0472514 | | | 0.0788888 | 0.0689496 |
| TG 46:1-FA18:1 | 0.1123859 | 0.1084171 | 0.1498065 | 0.1633013 | 0.1770966 | 0.0929974 | 0.1005454 | 0.0634258 | 0.1607259 | 0.1723469 |
| TG 46:2-FA12:0 | 0.6508832 | 0.2048748 | 0.3289597 | 0.4485211 | 0.3736369 | 0.311139 | 0.3939975 | 0.3841668 | 0.5255713 | 0.4285779 |
| TG 46:2-FA14:0 | 0.0991126 | 0.0906001 | 0.2318025 | | 0.0893532 | 0.0910819 | 0.0767672 | 0.07732 | 0.1828342 | 0.1399267 |
| TG 46:2-FA14:1 | 0.076704 | | | | | | | | 0.070086 | 0.0903958 |
| TG 46:2-FA16:0 | 0.4430956 | 0.1705867 | 0.3111197 | 0.3887908 | 0.5246875 | 0.2480102 | 0.2388865 | 0.2474638 | 0.4233055 | 0.4193091 |
| TG 46:2-FA16:1 | | | 0.0904431 | | | 0.0724963 | | | 0.0855473 | 0.1387915 |
| TG 46:2-FA18:1 | 0.4160415 | 0.1678214 | 0.2450614 | 0.5044297 | 0.4036486 | 0.1552966 | 0.1450948 | 0.1141913 | 0.2695914 | 0.5158581 |
| TG 46:2-FA18:2 | 0.2017807 | 0.091523 | 0.2465481 | 0.2567875 | 0.2034066 | 0.1464549 | 0.1895948 | 0.1464199 | 0.2649632 | 0.1797576 |
| TG 46:3-FA12:0 | 0.1381745 | | | 0.1126086 | 0.0883903 | 0.0773066 | 0.0848561 | 0.0922082 | 0.1088271 | |
| TG 46:3-FA14:1 | | | | | | | | | 0.0613896 | |
| TG 46:3-FA16:0 | 0.6788316 | 0.1339208 | 0.3350392 | 0.4920131 | 0.5295577 | 0.2877378 | 0.4126686 | 0.2586873 | 0.4185658 | 0.5127584 |
| TG 46:3-FA16:1 | | | | 0.0638268 | | | | | 0.0793072 | 0.1043067 |
| TG 46:3-FA18:1 | 0.3528204 | 0.16746 | 0.2820308 | 0.5718503 | 0.3835205 | 0.1651995 | 0.1777865 | 0.1388834 | 0.2214842 | 0.4363855 |
| TG 46:3-FA18:2 | 0.468874 | 0.1087361 | 0.3010553 | 0.6367655 | 0.4091612 | 0.221904 | 0.3005401 | 0.1976623 | 0.2623989 | 0.3784691 |
| TG 46:3-FA18:3 | 0.0875105 | | | 0.1000966 | 0.0541747 | | 0.056273 | | 0.0651102 | |
| TG 46:4-FA18:2 | 0.5155323 | 0.0903027 | 0.3184171 | 0.6893045 | 0.3709854 | 0.1850872 | 0.2852989 | 0.1505136 | 0.2493904 | 0.3506467 |
| TG 47:0-FA14:0 | | | | | | | | | 0.0762295 | |
| TG 47:0-FA15:0 | 0.2005785 | 0.1985321 | 0.2690262 | 0.2076032 | 0.2345931 | 0.1406018 | 0.1798515 | 0.2104911 | 0.2609898 | 0.2598596 |
| TG 47:0-FA16:0 | 0.5426101 | 0.5741733 | 0.7551568 | 0.6541993 | 0.6351872 | 0.2968109 | 0.5691743 | 0.4880738 | 0.6392146 | 0.5922538 |
| TG 47:0-FA17:0 | | | | | | | | 0.0476206 | | |
| TG 47:1-FA14:0 | | 0.0543014 | 0.0862871 | | 0.0533864 | | | | 0.0840778 | |
| TG 47:1-FA15:0 | 0.0957115 | 0.0942826 | 0.161099 | | 0.0939273 | 0.0862872 | 0.1008434 | | | 0.1072695 |
| TG 47:1-FA16:0 | 0.4446984 | 0.2015583 | 0.3698241 | 0.4264801 | 0.5230658 | 0.2388746 | 0.3448249 | 0.2414309 | 0.4504636 | 0.440958 |
| TG 47:1-FA16:1 | 0.0961255 | 0.08158 | 0.1215286 | 0.0962584 | 0.104857 | 0.0600698 | 0.1060861 | 0.1027826 | 0.0958309 | 0.1110828 |
| TG 47:1-FA17:0 | | | 0.0641987 | | 0.0589386 | | | | | |
| TG 47:1-FA18:1 | 0.1643729 | 0.1140772 | 0.1802719 | 0.2337445 | 0.2222174 | 0.0923914 | 0.0919258 | 0.0843376 | 0.1472466 | 0.2136095 |
| TG 47:2-FA14:0 | 0.088537 | 0.0623109 | 0.1292034 | | 0.0764457 | | 0.0843452 | 0.0815476 | 0.1110446 | 0.0654212 |
| TG 47:2-FA15:0 | | | 0.1246433 | 0.083743 | | | 0.0572343 | | 0.1035162 | |
| TG 47:2-FA16:1 | | | | | 0.0923118 | 0.0489793 | | | 0.0945018 | 0.0760301 |
| TG 47:2-FA18:1 | 0.275097 | 0.1144444 | 0.1930451 | 0.4206626 | 0.3151582 | 0.1336255 | 0.1165772 | 0.0889168 | 0.1761657 | 0.3007608 |
| TG 47:2-FA18:2 | 0.2921719 | 0.1026053 | 0.2918477 | 0.3610687 | 0.2781614 | 0.1348186 | 0.2073989 | 0.1578638 | 0.2303705 | 0.2546124 |
| TG 48:0-FA14:0 | 0.0493751 | 0.0666487 | 0.1134888 | 0.0825247 | 0.0779115 | 0.0428282 | 0.092696 | 0.0680355 | 0.1816822 | 0.0191499 |
| TG 48:0-FA16:0 | 2.7534405 | 4.3955647 | 6.1607697 | 4.5736306 | 3.8933574 | 2.1086492 | 3.138093 | 2.7043906 | 4.853563 | 5.0610716 |
| TG 48:0-FA18:0 | | 0.1055756 | 0.1653604 | 0.1127306 | 0.1261562 | 0.051056 | 0.0677276 | 0.0694678 | 0.2457794 | 0.0848753 |
| TG 48:1-FA12:0 | 0.1252709 | 0.0517724 | 0.0758893 | 0.0854853 | 0.0947891 | 0.0406114 | | 0.0439268 | 0.1019496 | 0.0913527 |
| TG 48:1-FA14:0 | 0.9995412 | 1.8083961 | 1.865916 | 0.8449367 | 1.1480614 | 1.0003608 | 0.7218023 | 0.5580473 | 1.572062 | 2.018514 |
| TG 48:1-FA14:1 | 0.0477201 | | 0.047941 | 0.0713331 | 0.0700983 | | | 0.0393204 | 0.0555163 | 0.0528701 |
| TG 48:1-FA16:0 | 2.0405402 | 4.6370009 | 3.8916214 | 1.6118365 | 2.8622109 | 2.73839 | 1.9191314 | 1.5996265 | 3.1925694 | 4.2654841 |
| TG 48:1-FA16:1 | 0.6952877 | 1.4461225 | 1.2221604 | 0.4657715 | 0.9930134 | 0.8423256 | 0.6720434 | 0.5443061 | 1.283588 | 1.2807369 |
| TG 48:1-FA18:0 | | | 0.0795692 | | 0.1158598 | 0.0548862 | 0.0524461 | | 0.0877372 | 0.0705816 |
| TG 48:1-FA18:1 | 0.7787915 | 1.7589387 | 1.8166574 | 0.8112683 | 1.2524199 | 1.0620822 | 0.7507951 | 0.5944079 | 1.4182056 | 1.7344076 |
| TG 48:2-FA12:0 | 0.3970947 | 0.3376209 | 0.2437396 | 0.2523404 | 0.167279 | 0.1623675 | 0.1654209 | 0.1714327 | 0.3345526 | 0.3257473 |
| TG 48:2-FA14:0 | 2.0010805 | 1.9062539 | 3.2025255 | 1.6771903 | 1.9059767 | 1.7990259 | 1.7047988 | 1.3962437 | 2.4949117 | 2.8652436 |
| TG 48:2-FA14:1 | 0.6847022 | 0.3794672 | 0.3968431 | 0.4708732 | 0.5731153 | 0.3816639 | 0.3514235 | 0.3144146 | 0.5139137 | 0.7453333 |
| TG 48:2-FA16:0 | 2.0433743 | 2.3304346 | 3.49249 | 1.6984654 | 2.3393499 | 2.3918109 | 1.6614768 | 1.4525692 | 2.718653 | 3.0998656 |
| TG 48:2-FA16:1 | 0.5968203 | 1.8302724 | 0.987529 | 0.2336536 | 0.8969549 | 1.2922653 | 0.3659542 | 0.4147741 | 0.9755838 | 2.1972302 |
| TG 48:2-FA18:0 | | | 0.0635851 | | 0.0720191 | | 0.0650125 | | | 0.0622438 |
| TG 48:2-FA18:1 | 0.5720919 | 0.8976441 | 0.5710451 | 0.4941951 | 0.6333573 | 0.5884974 | 0.2422681 | 0.2660683 | 0.5917771 | 1.2559002 |
| TG 48:2-FA18:2 | 1.4801931 | 1.2392369 | 2.9619746 | 1.4915418 | 1.7387954 | 1.5701288 | 1.42477 | 1.2391282 | 2.1839213 | 1.8344678 |
| TG 48:3-FA12:0 | 0.3851228 | 0.2566412 | 0.2118588 | 0.2814318 | 0.2020692 | 0.2696239 | 0.2005007 | 0.1948619 | 0.2894168 | 0.40815 |
| TG 48:3-FA14:0 | 0.424881 | 0.4116128 | 0.5789764 | 0.29361 | 0.4324918 | 0.5527731 | 0.2917236 | 0.2924427 | 0.5637032 | 0.8749282 |
| TG 48:3-FA14:1 | 1.3934521 | 0.3043166 | 0.5628195 | 0.974886 | 0.8360624 | 0.6930933 | 0.930863 | 0.8602564 | 0.9717942 | 0.8771685 |
| TG 48:3-FA16:0 | 0.6997185 | 0.3138557 | 0.5369865 | 0.4485177 | 0.5874734 | 0.595448 | 0.4604325 | 0.4015614 | 0.7049924 | 0.6731704 |
| TG 48:3-FA16:1 | 0.3565774 | 0.5934982 | 0.5483294 | 0.1896847 | 0.4481633 | 0.6190132 | 0.288331 | 0.2908959 | 0.5692208 | 1.0266474 |
| TG 48:3-FA18:1 | 0.4213388 | 0.2953098 | 0.3050195 | 0.4474203 | 0.3872825 | 0.334037 | 0.263591 | 0.1916853 | 0.4033493 | 0.5601617 |
| TG 48:3-FA18:2 | 0.6859165 | 0.5437107 | 0.6937216 | 0.6219475 | 0.7464646 | 0.8685957 | 0.5198304 | 0.4756054 | 0.7842935 | 1.0219585 |
| TG 48:3-FA18:3 | 0.1528386 | 0.1058676 | 0.2165618 | 0.1080445 | 0.1518134 | 0.1057038 | 0.1371678 | 0.1139168 | 0.1983303 | 0.1378195 |
| TG 48:4-FA12:0 | 0.2972507 | 0.1529137 | 0.1705499 | 0.2232372 | 0.160669 | 0.2014008 | 0.2055321 | 0.1847672 | 0.1964556 | 0.2201165 |
| TG 48:4-FA14:0 | | | 0.0853603 | | | 0.0672817 | 0.0715556 | | 0.1179534 | |
| TG 48:4-FA14:1 | 0.1534197 | 0.0587861 | 0.0938065 | 0.1102406 | 0.124657 | 0.1073334 | 0.0986554 | 0.0825117 | 0.1369782 | 0.1495946 |
| TG 48:4-FA16:0 | 0.40848 | 0.1688699 | 0.2796404 | 0.288791 | 0.2853261 | 0.2532585 | 0.306928 | 0.202964 | 0.3201865 | 0.2718439 |
| TG 48:4-FA16:1 | 0.0920518 | 0.0741824 | | | 0.0892606 | 0.0825153 | | 0.0528795 | | 0.1676272 |
| TG 48:4-FA18:1 | 0.2540828 | 0.1369334 | 0.2189343 | 0.3624007 | 0.2510331 | 0.174761 | 0.1502753 | 0.1191675 | 0.1878078 | 0.3116785 |
| TG 48:4-FA18:2 | 0.4732039 | 0.1621455 | 0.4044328 | 0.5528509 | 0.4170708 | 0.3839331 | 0.3976972 | 0.3110245 | 0.4154704 | 0.4472015 |
| TG 48:4-FA18:3 | 0.1073336 | | 0.0814066 | | 0.0547873 | 0.0682865 | | 0.0659196 | 0.093085 | 0.0904256 |
| TG 48:5-FA18:2 | 0.347008 | 0.1076721 | 0.2780205 | 0.4587389 | 0.2644705 | 0.1494983 | 0.2205383 | 0.1609232 | 0.2276579 | 0.2733704 |
| TG 48:5-FA18:3 | 0.1001189 | | | | | | | | 0.0756758 | |
| TG 49:0-FA15:0 | 0.0734338 | 0.1323398 | 0.0864855 | 0.0582296 | 0.122115 | 0.056152 | 0.1238428 | 0.1228213 | 0.1559718 | 0.1154918 |
| TG 49:0-FA16:0 | 0.7378114 | 1.0592385 | 1.1274284 | 0.7544857 | 0.943906 | 0.6194326 | 0.8706069 | 0.6329846 | 1.1564192 | 0.7203938 |
| TG 49:0-FA17:0 | 0.2227925 | 0.3188869 | 0.3191595 | 0.20126 | 0.3034836 | 0.1848609 | 0.2704531 | 0.2128386 | 0.356882 | 0.3409097 |
| TG 49:0-FA18:0 | 0.1430964 | 0.2005765 | 0.2070365 | 0.1262717 | 0.1817314 | 0.1166648 | 0.2194183 | 0.1889577 | 0.248535 | 0.1388457 |
| TG 49:1-FA14:0 | 0.0993252 | 0.0799749 | 0.1600365 | 0.0831909 | 0.1253416 | 0.0378389 | 0.0818288 | 0.0582303 | 0.1516523 | 0.1098989 |
| TG 49:1-FA15:0 | 0.8588206 | 0.9840467 | 1.0832613 | 0.950621 | 0.9694726 | 0.563558 | 0.5926722 | 0.5847068 | 0.7278473 | 1.1457185 |
| TG 49:1-FA16:0 | 1.1468523 | 1.5986094 | 1.782591 | 1.2496827 | 1.3228839 | 0.8766652 | 0.9691569 | 0.8892071 | 1.4568322 | 1.404648 |
| TG 49:1-FA16:1 | 0.1436305 | 0.1750164 | 0.1887086 | 0.1136821 | 0.1019682 | 0.1001402 | 0.1492172 | 0.1753919 | 0.2401599 | 0.1217076 |
| TG 49:1-FA17:0 | 0.1189985 | 0.1277979 | 0.1783993 | 0.0796472 | 0.1405099 | 0.0798273 | 0.1141529 | 0.096198 | 0.204804 | 0.1647506 |
| TG 49:1-FA18:1 | 1.0348428 | 1.1337239 | 1.6772912 | 1.1442075 | 1.0907673 | 0.5606192 | 0.7144013 | 0.6632459 | 1.0172345 | 1.027412 |
| TG 49:2-FA14:0 | 0.2145119 | 0.1722476 | 0.2503508 | 0.168546 | 0.1626023 | 0.1098454 | 0.1525372 | 0.1260769 | 0.2166038 | 0.2360454 |
| TG 49:2-FA15:0 | 1.6227538 | 1.4547832 | 2.050169 | 1.7318549 | 1.7307272 | 1.004687 | 1.2337309 | 1.2549332 | 1.2025414 | 1.8530531 |
| TG 49:2-FA16:0 | 1.8221277 | 1.7183657 | 2.6825768 | 2.240119 | 1.8641581 | 1.1740291 | 1.3234959 | 1.3674175 | 1.7318095 | 2.133887 |
| TG 49:2-FA16:1 | 0.2144577 | 0.4450461 | 0.2860778 | 0.0996509 | 0.2826227 | 0.2285567 | 0.1288953 | 0.1441486 | 0.2570447 | 0.4535755 |
| TG 49:2-FA17:0 | 0.1062345 | 0.0956931 | 0.1321728 | 0.1056952 | 0.0944333 | 0.0655218 | 0.0759037 | 0.1007356 | 0.1178325 | 0.1103354 |
| TG 49:2-FA18:1 | 0.4145115 | 0.4928587 | 0.4467175 | 0.402673 | 0.4422208 | 0.2923459 | 0.2501966 | 0.2165207 | 0.4661841 | 0.5920315 |
| TG 49:2-FA18:2 | 1.75486 | 1.6671669 | 3.1336815 | 2.1389839 | 1.6501953 | 0.9920389 | 1.2381624 | 1.3342193 | 1.6816282 | 1.6579544 |
| TG 49:3-FA15:0 | 0.3240201 | 0.2677109 | 0.3684182 | 0.2473657 | 0.3138966 | 0.2928332 | 0.223192 | 0.2549106 | 0.3100836 | 0.3026984 |
| TG 49:3-FA16:0 | 0.5316258 | 0.2596392 | 0.5021059 | 0.4158612 | 0.4175945 | 0.3184561 | 0.4339075 | 0.3058927 | 0.5179608 | 0.4280091 |
| TG 49:3-FA16:1 | 0.2250679 | 0.3013429 | 0.2751478 | 0.1469854 | 0.2844688 | 0.2659902 | 0.1538777 | 0.2195063 | 0.2996897 | 0.3482659 |
| TG 49:3-FA18:2 | 0.5781916 | 0.4183823 | 0.6062579 | 0.5341665 | 0.4792528 | 0.414847 | 0.4214309 | 0.3497939 | 0.6236318 | 0.5435552 |
| TG 49:3-FA18:3 | 0.1164144 | 0.0928244 | 0.2440577 | 0.1254754 | 0.103698 | 0.0704969 | 0.1284138 | | 0.150711 | |
| TG 50:0-FA14:0 | | | 0.079764 | | 0.0465159 | | | | 0.0862267 | 0.0433651 |
| TG 50:0-FA16:0 | 1.2972365 | 2.1006183 | 2.5195502 | 1.5092982 | 1.7411295 | 1.009738 | 1.7270204 | 1.3386907 | 2.5133704 | 1.8940153 |
| TG 50:0-FA18:0 | 0.7447903 | 1.261576 | 1.4977706 | 0.7415518 | 1.0337665 | 0.7611723 | 1.0185777 | 0.806925 | 1.630502 | 1.1687823 |
| TG 50:1-FA14:0 | 0.2280428 | 0.2565366 | 0.5431906 | 0.1961602 | 0.3369443 | 0.0929006 | 0.2679266 | 0.1786895 | 0.5314728 | 0.2663936 |
| TG 50:1-FA16:0 | 19.816593 | 43.176568 | 34.193209 | 25.874947 | 24.238998 | 22.263958 | 13.953563 | 13.08588 | 21.236738 | 40.008273 |
| TG 50:1-FA16:1 | 0.2037979 | 0.2816164 | 0.4777937 | 0.1795971 | 0.2574264 | 0.2461513 | 0.267766 | 0.2447563 | 0.4788656 | 0.2247379 |
| TG 50:1-FA18:0 | 0.2760351 | 0.6167014 | 0.7007921 | 0.2868548 | 0.4281455 | 0.3638359 | 0.4392055 | 0.3598049 | 0.8311255 | 0.5578527 |
| TG 50:1-FA18:1 | 10.039643 | 21.238661 | 18.44746 | 13.634336 | 13.30252 | 10.994402 | 7.4845042 | 6.39995 | 11.751632 | 19.412519 |
| TG 50:1-FA20:1 | 0.1674062 | 0.1171634 | 0.3961039 | 0.1829244 | 0.225437 | 0.0716336 | 0.1910052 | 0.1035584 | 0.3293261 | 0.1114926 |
| TG 50:2-FA14:0 | 1.203957 | 5.1946356 | 2.4170656 | 1.0327405 | 1.5328692 | 1.8196346 | 0.7455215 | 0.8118441 | 1.6781443 | 4.8427621 |
| TG 50:2-FA14:1 | 0.1696579 | | 0.1221657 | 0.1143741 | 0.1568221 | 0.0690482 | 0.1005386 | 0.0691437 | 0.1337592 | 0.129027 |
| TG 50:2-FA16:0 | 47.883399 | 59.126346 | 67.806945 | 53.292937 | 48.993033 | 49.597634 | 38.480151 | 36.992809 | 44.484459 | 77.766531 |
| TG 50:2-FA16:1 | 6.8589073 | 20.754029 | 8.5147053 | 3.5180885 | 8.6447868 | 11.373878 | 3.2965343 | 4.0148766 | 6.5032516 | 19.467548 |
| TG 50:2-FA18:0 | 0.287905 | 0.331645 | 0.7693579 | 0.3300469 | 0.3525628 | 0.2116461 | 0.352045 | 0.3104388 | 0.7304497 | 0.3911349 |
| TG 50:2-FA18:1 | 8.2372625 | 28.448226 | 11.553806 | 4.8811066 | 10.428295 | 13.71995 | 3.8951344 | 4.7797398 | 8.7076716 | 26.687799 |
| TG 50:2-FA18:2 | 20.475887 | 18.193844 | 31.47809 | 26.819297 | 21.390551 | 18.316129 | 18.079971 | 15.778766 | 19.570168 | 27.898125 |
| TG 50:2-FA20:2 | | | 0.0642689 | | | | | | | |
| TG 50:3-FA14:0 | 3.6889518 | 6.1528072 | 5.3660273 | 3.2609404 | 3.7074949 | 4.6631756 | 2.1599035 | 2.1825274 | 3.6411351 | 8.5016605 |
| TG 50:3-FA14:1 | 0.290925 | 0.3272196 | 0.1994229 | 0.3095209 | 0.2885874 | 0.1887497 | 0.1555151 | 0.1314032 | 0.2615617 | 0.3860457 |
| TG 50:3-FA16:0 | 14.889003 | 16.524843 | 13.660646 | 8.0592916 | 15.239049 | 21.081459 | 10.276623 | 11.24919 | 14.130457 | 21.660182 |
| TG 50:3-FA16:1 | 13.494582 | 17.966866 | 11.986707 | 7.0451952 | 14.458107 | 19.945893 | 8.3860187 | 9.6231256 | 12.389297 | 23.373845 |
| TG 50:3-FA18:0 | 0.1073664 | 0.0912536 | 0.1187388 | 0.0831952 | 0.1068622 | 0.071576 | 0.0743522 | 0.0739974 | 0.1430893 | |
| TG 50:3-FA18:1 | 4.685391 | 8.4202589 | 6.9450188 | 3.8519058 | 4.5877537 | 6.1545663 | 2.6070587 | 2.8039125 | 4.9954417 | 10.622178 |
| TG 50:3-FA18:2 | 14.532261 | 17.723644 | 15.083549 | 8.7231867 | 14.864219 | 20.114322 | 9.0596485 | 10.390846 | 13.261434 | 23.484632 |
| TG 50:3-FA18:3 | 0.8410076 | 0.89652 | 1.7351193 | 0.8310877 | 1.0103353 | 0.970943 | 1.2415511 | 0.7998923 | 1.3623018 | 0.9265492 |
| TG 50:3-FA20:3 | 0.0748795 | 0.0572235 | | | | | 0.0618139 | | | |
| TG 50:4-FA14:0 | 2.5080082 | 1.8298458 | 2.6978107 | 2.2317083 | 1.9764272 | 2.866054 | 2.0094605 | 1.8679771 | 2.2635921 | 3.1828434 |
| TG 50:4-FA14:1 | 0.5002642 | 0.2530053 | 0.2121926 | 0.4712349 | 0.3428922 | 0.3651779 | 0.2817426 | 0.2219477 | 0.3625766 | 0.5249182 |
| TG 50:4-FA16:0 | 1.3148989 | 0.8659715 | 1.1473959 | 0.9412467 | 1.1071217 | 1.554895 | 1.1497707 | 0.9954239 | 1.2870342 | 1.2431385 |
| TG 50:4-FA16:1 | 1.4533781 | 2.1652245 | 1.1596593 | 0.6486202 | 1.71457 | 2.6128276 | 0.8704885 | 0.9271875 | 1.5635591 | 3.2812115 |
| TG 50:4-FA18:1 | 0.4939 | 0.4865104 | 0.4805803 | 0.5160117 | 0.4402639 | 0.493933 | 0.3351168 | 0.2487088 | 0.5142049 | 0.6201435 |
| TG 50:4-FA18:2 | 4.5526197 | 3.433856 | 4.8297907 | 4.0694295 | 3.8921967 | 5.1725273 | 3.3820433 | 2.9615506 | 4.0556719 | 5.7685012 |
| TG 50:4-FA18:3 | 0.6482225 | 0.6654522 | 0.7198889 | 0.3283022 | 0.5952086 | 1.0208481 | 0.4644632 | 0.5082592 | 0.7829618 | 0.862023 |
| TG 50:4-FA20:3 | 0.0752281 | | | | 0.0883016 | 0.0536468 | 0.062522 | | 0.0801114 | |
| TG 50:4-FA20:4 | | 0.0592924 | 0.1454063 | 0.0701606 | 0.0791335 | | 0.0639147 | | 0.0872531 | |
| TG 50:5-FA14:0 | 0.2922235 | 0.1658999 | 0.3095511 | 0.1848136 | 0.2181387 | 0.3404016 | 0.246626 | 0.2390591 | 0.2959279 | 0.2632029 |
| TG 50:5-FA16:0 | 0.3098746 | 0.1998958 | 0.3409777 | 0.2591014 | 0.2280541 | 0.2639346 | 0.3250979 | 0.2748463 | 0.3712231 | 0.2862331 |
| TG 50:5-FA16:1 | 0.1686659 | 0.1604606 | 0.1854164 | 0.0978425 | 0.1628958 | 0.2614679 | 0.1177079 | | 0.2644089 | 0.2418176 |
| TG 50:5-FA18:1 | 0.2549601 | 0.1591007 | 0.1869877 | 0.2192397 | 0.2124727 | 0.1726675 | 0.1569438 | 0.1345602 | 0.2101263 | 0.2319475 |
| TG 50:5-FA18:2 | 0.558951 | 0.2896449 | 0.5075382 | 0.5761836 | 0.4787719 | 0.5759693 | 0.4680289 | 0.3593043 | 0.5725941 | 0.5971415 |
| TG 50:5-FA18:3 | 0.3098746 | 0.2338917 | 0.347263 | 0.250042 | 0.2294705 | 0.4094686 | 0.2550337 | 0.3006132 | 0.3869826 | 0.3470988 |
| TG 50:5-FA20:4 | | | | | | | | | | 0.0600045 |
| TG 50:5-FA20:5 | 0.0915241 | 0.0758645 | 0.0909715 | | | 0.07263 | 0.0910835 | 0.1065666 | 0.1478666 | 0.0806059 |
| TG 51:0-FA16:0 | 0.7243584 | 1.2868787 | 1.0428648 | 0.8096233 | 1.2495329 | 0.8192845 | 1.0887807 | 0.8049975 | 1.4443089 | 0.8151671 |
| TG 51:0-FA17:0 | 0.0531412 | 0.1412124 | 0.115521 | 0.1032015 | 0.119835 | 0.1276689 | 0.1465288 | 0.0785276 | 0.2239563 | 0.1221499 |
| TG 51:0-FA18:0 | 0.1397367 | 0.3006806 | 0.2500178 | 0.1576755 | 0.1878507 | 0.1863272 | 0.2778127 | 0.1636837 | 0.352924 | 0.162541 |
| TG 51:1-FA15:0 | 0.3027866 | 0.2957578 | 0.3129546 | 0.2076588 | 0.3297437 | 0.1606357 | 0.2397332 | 0.1903689 | 0.3228311 | 0.2434766 |
| TG 51:1-FA16:0 | 2.0243127 | 2.2431716 | 3.0356763 | 2.2168965 | 2.5230582 | 1.3866922 | 1.5037301 | 1.0479966 | 2.1507307 | 2.4854733 |
| TG 51:1-FA17:0 | 1.1714539 | 1.4942409 | 1.4586588 | 1.0152967 | 1.2556514 | 0.8880425 | 0.8149463 | 0.6968515 | 1.2966664 | 1.6017105 |
| TG 51:1-FA18:0 | 0.2560935 | 0.3910887 | 0.4811509 | 0.2832377 | 0.2687661 | 0.2491109 | 0.3221217 | 0.2644555 | 0.4162983 | 0.2048469 |
| TG 51:1-FA18:1 | 1.2698643 | 1.8172131 | 2.134536 | 1.4026611 | 1.4408459 | 0.9886625 | 1.0054248 | 0.7879597 | 1.614459 | 1.1541782 |
| TG 51:2-FA15:0 | 1.0309176 | 2.1259843 | 1.3980832 | 1.1494139 | 1.2165676 | 0.9792222 | 0.7029982 | 0.7818795 | 0.9777499 | 1.7731044 |
| TG 51:2-FA16:0 | 5.3401864 | 5.6277642 | 6.2577672 | 5.6602063 | 6.0690508 | 3.9367435 | 3.246906 | 2.7850408 | 4.6752402 | 6.7205942 |
| TG 51:2-FA16:1 | 0.3431458 | 0.5281028 | 0.4264111 | 0.2623433 | 0.4516151 | 0.3133023 | 0.2230474 | 0.1911445 | 0.4436216 | 0.6552935 |
| TG 51:2-FA17:0 | 2.4214159 | 2.1808581 | 2.5603341 | 2.0924809 | 2.1171157 | 1.6775066 | 1.8721471 | 1.6931947 | 2.3589542 | 2.578834 |
| TG 51:2-FA18:1 | 3.0245913 | 6.6679931 | 4.2422801 | 2.8367542 | 3.1934187 | 3.0261265 | 1.5897922 | 1.5991999 | 2.6418212 | 5.2688449 |
| TG 51:2-FA18:2 | 2.5097916 | 2.8300374 | 4.4536591 | 2.8212391 | 2.5674984 | 1.8376409 | 1.8137788 | 1.4763279 | 2.6616358 | 1.9594622 |
| TG 51:3-FA15:0 | 2.795581 | 2.9600152 | 2.6390398 | 2.8729273 | 2.5089022 | 2.2929344 | 1.5198829 | 1.6358459 | 1.8146891 | 3.3686523 |
| TG 51:3-FA16:1 | 0.9950146 | 0.9952898 | 0.7776376 | 0.5492535 | 1.0895043 | 0.7866247 | 0.4926731 | 0.3633465 | 0.7391704 | 1.4058021 |
| TG 51:3-FA17:0 | 0.3632241 | 0.3440495 | 0.375295 | 0.2933921 | 0.3555152 | 0.3469939 | 0.302065 | 0.2680125 | 0.4518912 | 0.4628178 |
| TG 51:3-FA18:2 | 4.4295434 | 5.0940595 | 4.9203296 | 4.2283928 | 3.9703195 | 3.7720452 | 2.4645783 | 2.3524064 | 3.3201753 | 5.1530976 |
| TG 51:3-FA18:3 | 0.12607 | 0.1459891 | 0.2375521 | | 0.1398841 | 0.1116161 | 0.1629805 | 0.1190375 | 0.260644 | 0.1180111 |
| TG 51:4-FA15:0 | 2.3394136 | 1.5381279 | 1.8886302 | 2.559078 | 1.8443986 | 1.7980873 | 1.681416 | 1.5873167 | 1.4031 | 2.2168645 |
| TG 51:4-FA16:1 | 0.3313673 | 0.409984 | 0.2384634 | 0.2103299 | 0.3869368 | 0.384324 | 0.1644555 | 0.1477247 | 0.3171148 | 0.6091384 |
| TG 51:4-FA18:2 | 5.0051211 | 3.1802032 | 4.3800944 | 5.1123882 | 3.6002654 | 3.3675184 | 3.1700635 | 2.9778988 | 2.975968 | 4.3892419 |
| TG 51:4-FA18:3 | 0.3147024 | 0.2871095 | 0.4141751 | 0.2645782 | 0.2279521 | 0.283227 | 0.2670283 | 0.2369589 | 0.3111085 | 0.2639553 |
| TG 51:4-FA20:4 | | 0.1004323 | 0.1351293 | | 0.089003 | 0.0577931 | | 0.0762254 | 0.0992091 | 0.1051145 |
| TG 51:5-FA18:2 | 0.4800409 | 0.3204618 | 0.5626048 | 0.5552779 | 0.4068781 | 0.3804664 | 0.4209365 | 0.3257656 | 0.5082941 | 0.4292639 |
| TG 51:5-FA18:3 | 0.2935788 | 0.1870507 | 0.3067309 | 0.2767227 | 0.2363904 | 0.234517 | 0.2919627 | 0.2620604 | 0.3010801 | 0.2279424 |
| TG 52:0-FA16:0 | 0.6703541 | 1.0702272 | 1.0959145 | 0.6976987 | 0.6404262 | 0.5658004 | 0.8468379 | 0.6299783 | 1.0191968 | 0.8170634 |
| TG 52:0-FA18:0 | 0.3805651 | 1.0869889 | 0.6793818 | 0.3873852 | 0.5550019 | 0.5856261 | 0.6197053 | 0.4846341 | 0.7444737 | 0.8040108 |
| TG 52:0-FA20:0 | 2.2696139 | 0.8995301 | 2.4487278 | 2.5714265 | 2.1142652 | 1.2671858 | 1.7222486 | 1.374 | 1.4717425 | 1.8955617 |
| TG 52:1-FA16:0 | 6.364895 | 7.2614496 | 10.133321 | 6.3603217 | 6.950468 | 4.6697157 | 4.329632 | 3.6387138 | 7.0835268 | 6.5487526 |
| TG 52:1-FA16:1 | 0.1093266 | 0.166672 | 0.2365615 | 0.0617874 | 0.1287012 | 0.0874592 | 0.1292732 | 0.1015559 | 0.2162112 | 0.1148005 |
| TG 52:1-FA18:0 | 3.7813277 | 6.4658367 | 7.035373 | 4.4787565 | 4.0990403 | 3.7687078 | 2.9290943 | 3.0930338 | 5.1775429 | 6.083144 |
| TG 52:1-FA18:1 | 3.464447 | 5.7459677 | 6.1917171 | 4.2467868 | 3.8402123 | 3.085785 | 2.7416302 | 2.8389712 | 4.8614061 | 5.1013033 |
| TG 52:1-FA20:0 | 1.3456449 | 0.7170765 | 1.2608085 | 0.8729395 | 1.2279953 | 1.1725646 | 0.7629686 | 0.7352819 | 0.9694421 | 1.3845818 |
| TG 52:1-FA20:1 | 1.7988817 | 1.2110345 | 2.9305055 | 1.6303721 | 2.0002133 | 0.8125525 | 1.3242021 | 0.7333389 | 1.8041144 | 1.0438296 |
| TG 52:2-FA14:0 | 0.2799451 | 0.2600406 | 0.5855801 | 0.3328903 | 0.3031729 | 0.1308327 | 0.2009659 | 0.1317174 | 0.3430005 | 0.3304568 |
| TG 52:2-FA16:0 | 56.054757 | 174.83084 | 63.6901 | 47.360342 | 59.48941 | 77.015167 | 27.648253 | 31.837171 | 41.002489 | 141.97457 |
| TG 52:2-FA16:1 | 1.0992731 | 1.3414475 | 1.438104 | 0.6474591 | 1.1761214 | 0.8736725 | 0.7083919 | 0.5397833 | 1.320191 | 1.2469593 |
| TG 52:2-FA18:0 | 7.5815775 | 8.7109799 | 13.819821 | 8.1669343 | 8.0720155 | 6.8806264 | 7.4110417 | 7.7911169 | 9.9539705 | 9.8787295 |
| TG 52:2-FA18:1 | 87.948614 | 294.689 | 96.708595 | 77.415744 | 96.127098 | 127.38318 | 39.530185 | 44.251317 | 62.112088 | 231.42042 |
| TG 52:2-FA18:2 | 6.3399089 | 6.0114256 | 11.330015 | 7.0094283 | 6.5998209 | 5.7577779 | 6.1226285 | 6.0892277 | 8.176958 | 7.3455612 |
| TG 52:2-FA20:0 | 0.6288012 | 0.2566581 | 0.7027177 | 0.5656459 | 0.4911072 | 0.4193806 | 0.4076992 | 0.3499377 | 0.4386708 | 0.4838884 |
| TG 52:2-FA20:1 | 0.8847236 | 0.7583296 | 1.3465042 | 0.5208319 | 1.0401125 | 0.5573363 | 0.6218949 | 0.3905751 | 1.0440818 | 0.8737632 |
| TG 52:2-FA20:2 | 0.1707533 | 0.3077088 | 0.3803919 | 0.1423676 | 0.2212559 | 0.1591218 | 0.1940534 | 0.1415386 | 0.2358255 | 0.2148196 |
| TG 52:3-FA14:0 | 0.5155855 | 0.2927594 | 0.7682097 | 0.2988149 | 0.4148074 | 0.2125143 | 0.2854717 | 0.2306044 | 0.4929612 | 0.4063484 |
| TG 52:3-FA16:0 | 182.19591 | 239.06705 | 138.53593 | 153.70884 | 160.38476 | 216.97523 | 111.21175 | 113.69372 | 120.22616 | 265.1335 |
| TG 52:3-FA16:1 | 5.7447127 | 27.742405 | 6.3109452 | 3.6373486 | 6.8475049 | 9.2803092 | 2.4479963 | 2.6884481 | 4.6184516 | 19.792342 |
| TG 52:3-FA18:0 | 1.2763495 | 1.7133461 | 2.0910342 | 0.9627955 | 1.3336419 | 1.4781271 | 1.1410759 | 1.3029941 | 1.8938792 | 1.5456052 |
| TG 52:3-FA18:1 | 174.88873 | 258.65103 | 141.06041 | 148.60866 | 156.60496 | 209.1406 | 104.05438 | 107.80933 | 117.94261 | 271.03799 |
| TG 52:3-FA18:2 | 156.7565 | 199.49521 | 125.7156 | 138.38149 | 137.71787 | 179.6046 | 94.968162 | 96.54881 | 104.45033 | 223.20914 |
| TG 52:3-FA18:3 | 0.2698081 | 0.3688867 | 0.5193326 | 0.2492442 | 0.2585446 | 0.1682275 | 0.2685996 | 0.3452773 | 0.4283006 | 0.3007508 |
| TG 52:3-FA20:0 | 0.44056 | 0.4847855 | 0.3386824 | 0.431655 | 0.4327753 | 0.4525288 | 0.2818273 | 0.2893682 | 0.3708145 | 0.5851544 |
| TG 52:3-FA20:1 | 0.6060734 | 0.4079722 | 0.975902 | 0.4417718 | 0.5701668 | 0.3431968 | 0.4305287 | 0.3085008 | 0.6932768 | 0.5000196 |
| TG 52:3-FA20:2 | 0.3682176 | 0.5671792 | 0.4811317 | 0.2352016 | 0.2938532 | 0.3083392 | 0.1805363 | 0.2173466 | 0.265003 | 0.5482249 |
| TG 52:3-FA20:3 | 0.21439 | 0.37042 | 0.3683485 | 0.2083149 | 0.2921846 | 0.2531652 | 0.1886408 | 0.2295725 | 0.2251874 | 0.3639214 |
| TG 52:3-FA22:1 | 0.1153103 | 0.1205317 | 0.110085 | 0.1091036 | 0.0903327 | 0.1055136 | 0.0737157 | | | 0.1729962 |
| TG 52:4-FA14:0 | 0.1425002 | 0.2015919 | 0.2707345 | 0.1431221 | 0.1373838 | 0.1279126 | 0.124283 | 0.0969871 | 0.1707523 | 0.2439294 |
| TG 52:4-FA16:0 | 164.97308 | 96.126707 | 95.17048 | 141.50997 | 117.40282 | 166.11036 | 139.80346 | 118.99512 | 101.93184 | 141.8608 |
| TG 52:4-FA16:1 | 14.661369 | 32.417835 | 12.281169 | 10.459327 | 15.478799 | 22.122878 | 6.7837801 | 7.0125935 | 10.560282 | 31.94233 |
| TG 52:4-FA18:0 | 0.2172855 | 0.2005063 | 0.320214 | 0.1750467 | 0.2235736 | 0.2535724 | 0.2105125 | 0.1764921 | 0.3176523 | 0.222374 |
| TG 52:4-FA18:1 | 23.053441 | 39.407592 | 18.411478 | 16.323286 | 21.559979 | 31.430631 | 14.036963 | 13.09534 | 17.899911 | 39.730115 |
| TG 52:4-FA18:2 | 265.26164 | 166.67548 | 163.00083 | 234.56763 | 190.23737 | 264.65094 | 217.1566 | 187.97273 | 167.94578 | 237.57316 |
| TG 52:4-FA18:3 | 14.133623 | 12.927781 | 10.202724 | 10.821798 | 11.121105 | 17.028967 | 11.198066 | 10.035852 | 10.785906 | 14.95778 |
| TG 52:4-FA20:0 | 1.1599922 | 0.612262 | 0.6833661 | 1.0438558 | 0.8711335 | 1.1711938 | 0.9621594 | 0.7778244 | 0.7131494 | 1.0503971 |
| TG 52:4-FA20:2 | 0.275465 | 0.2284454 | 0.2827026 | 0.236264 | 0.1824336 | 0.2599723 | 0.1846611 | 0.1697043 | 0.1925093 | 0.2846369 |
| TG 52:4-FA20:3 | 0.4570581 | 0.5331551 | 0.4725284 | 0.3256205 | 0.3656192 | 0.5532387 | 0.292013 | 0.3300576 | 0.2957242 | 0.6718116 |
| TG 52:4-FA20:4 | 0.5811536 | 0.8383606 | 1.3904036 | 0.5212737 | 0.720565 | 0.6683005 | 0.6252595 | 0.5881955 | 0.6729511 | 1.1060803 |
| TG 52:4-FA22:1 | 0.2749462 | 0.1878531 | 0.1784791 | 0.2206374 | 0.1942294 | 0.2852261 | 0.140524 | 0.1728499 | 0.1540976 | 0.3114158 |
| TG 52:4-FA22:4 | 0.1129025 | 0.054013 | 0.091529 | 0.092067 | 0.0840044 | 0.076018 | 0.0926917 | 0.0953788 | 0.0805129 | 0.1345069 |
| TG 52:5-FA14:0 | 0.1570072 | 0.1467619 | 0.2324862 | 0.1131724 | 0.0909958 | 0.1168779 | 0.1107685 | 0.1119795 | 0.1512483 | 0.1916591 |
| TG 52:5-FA16:0 | 14.077899 | 8.0531359 | 8.07947 | 8.3175507 | 9.0251984 | 17.194059 | 14.769071 | 11.942026 | 11.872323 | 9.5300883 |
| TG 52:5-FA16:1 | 11.231824 | 10.967428 | 7.8563827 | 8.6119833 | 9.6824379 | 14.65347 | 6.5134339 | 6.1873996 | 7.8891513 | 13.785848 |
| TG 52:5-FA18:1 | 1.9864408 | 2.5566195 | 1.9210699 | 1.6762429 | 1.7841619 | 2.6306562 | 1.3882829 | 1.2026319 | 1.988055 | 2.687852 |
| TG 52:5-FA18:2 | 26.243446 | 20.898261 | 17.870329 | 18.984724 | 21.093778 | 33.460633 | 19.951623 | 17.641416 | 20.19249 | 26.420633 |
| TG 52:5-FA18:3 | 16.674266 | 9.7059837 | 10.020762 | 10.710157 | 11.114387 | 19.540953 | 16.580816 | 13.389512 | 13.28874 | 12.106762 |
| TG 52:5-FA20:3 | 0.1847129 | 0.1788521 | 0.2257053 | 0.1600864 | 0.1527014 | 0.1698999 | 0.1068331 | 0.1056028 | 0.1541725 | 0.2208701 |
| TG 52:5-FA20:4 | 0.3608894 | 0.5056436 | 0.9769243 | 0.2466869 | 0.3755288 | 0.5972979 | 0.2387131 | 0.3687012 | 0.4559578 | 0.7149326 |
| TG 52:5-FA20:5 | 0.7405614 | 0.9024403 | 1.0285746 | 0.5251315 | 0.6634814 | 0.7968871 | 1.1422342 | 0.7797892 | 1.2404503 | 0.6445534 |
| TG 52:5-FA22:5 | 0.442946 | 0.2557103 | 0.2857228 | 0.229579 | 0.2222095 | 0.3873289 | 0.412595 | 0.3293323 | 0.3394297 | 0.3244264 |
| TG 52:6-FA14:0 | 0.222633 | 0.1877404 | 0.2571129 | 0.1443376 | 0.1506548 | 0.2081154 | 0.196329 | 0.2108089 | 0.2739859 | 0.353287 |
| TG 52:6-FA16:0 | 1.0799661 | 0.589091 | 0.6736925 | 0.6524702 | 0.6633147 | 1.3420845 | 1.2249385 | 1.1477383 | 1.2427443 | 0.7275909 |
| TG 52:6-FA16:1 | 1.3558552 | 1.3280897 | 0.9288204 | 0.7041337 | 1.038649 | 2.1429576 | 1.030369 | 0.9340005 | 1.3663482 | 1.5258633 |
| TG 52:6-FA18:1 | 0.3145821 | 0.4096499 | 0.4559209 | 0.2554782 | 0.2582942 | 0.3375333 | 0.2558402 | 0.2037068 | 0.4396995 | 0.3335349 |
| TG 52:6-FA18:2 | 2.4168721 | 1.6298651 | 2.3847222 | 2.1994912 | 1.9078113 | 2.9148764 | 2.1051628 | 1.7362455 | 2.2903792 | 2.1584152 |
| TG 52:6-FA18:3 | 1.9415205 | 1.3266991 | 1.1489733 | 1.106231 | 1.5050995 | 2.784962 | 1.570632 | 1.3790416 | 1.7979208 | 1.6217555 |
| TG 52:6-FA20:4 | 0.1980712 | 0.1605634 | 0.4644788 | 0.2265417 | 0.1764592 | 0.2266663 | 0.167548 | 0.1915024 | 0.2370381 | 0.3256309 |
| TG 52:6-FA20:5 | 0.7196177 | 0.7313897 | 0.6182325 | 0.3377007 | 0.5069501 | 0.8996269 | 0.5523817 | 0.7398128 | 1.0924773 | 0.9377667 |
| TG 52:6-FA22:6 | 0.4139716 | 0.5634738 | 0.4706491 | 0.2721707 | 0.4408626 | 0.5048169 | 0.4985286 | 0.5064311 | 0.5890602 | 0.6832592 |
| TG 52:7-FA16:0 | 0.6909957 | 0.9012393 | 0.8469388 | 0.596717 | 0.9396648 | 0.7146845 | 0.9370235 | 0.6632089 | 1.1032828 | 0.7319333 |
| TG 52:7-FA18:1 | 0.109138 | 0.1530672 | 0.1404644 | 0.1002381 | 0.0717649 | 0.071793 | 0.0676405 | 0.1058484 | 0.1221152 | 0.1019236 |
| TG 52:7-FA20:5 | 0.3549029 | 0.2599773 | 0.3229012 | 0.2963884 | 0.2534296 | 0.4804131 | 0.3423982 | 0.4536886 | 0.5818217 | 0.4221354 |
| TG 52:7-FA22:6 | 0.2490547 | 0.1765621 | 0.1686797 | 0.1945049 | 0.1810212 | 0.240837 | 0.1690502 | 0.2473335 | 0.2560015 | 0.3884991 |
| TG 52:8-FA16:1 | 0.1214252 | 0.1022354 | 0.1270967 | 0.0722262 | 0.1323832 | 0.1195702 | 0.1221626 | 0.0991715 | 0.189569 | 0.1052 |
| TG 52:8-FA18:2 | 0.122768 | 0.1172708 | 0.1796123 | 0.1303247 | 0.0730547 | 0.1114209 | 0.1416888 | 0.1368251 | 0.1533692 | 0.1011928 |
| TG 53:0-FA16:0 | 4.3931528 | 3.2693566 | 2.6320047 | 2.730951 | 2.2367062 | 4.8607764 | 5.5053026 | 5.0817361 | 5.7439224 | 3.6919958 |
| TG 53:1-FA16:0 | 1.5716854 | 1.7477187 | 2.0153529 | 1.4205112 | 1.9313646 | 1.2932301 | 1.495168 | 0.9028301 | 1.9805122 | 1.6242382 |
| TG 53:1-FA17:0 | 0.358956 | 0.3715663 | 0.3781522 | 0.2530069 | 0.3848284 | 0.2372725 | 0.2845626 | 0.2094332 | 0.3740054 | 0.2409881 |
| TG 53:1-FA18:0 | 0.4158121 | 0.6765245 | 0.8164335 | 0.4686207 | 0.5741599 | 0.4138225 | 0.4770674 | 0.3838772 | 0.6517742 | 0.4500253 |
| TG 53:1-FA18:1 | 0.8717256 | 1.4820837 | 1.5594556 | 0.9655506 | 1.2182393 | 0.9709256 | 0.8722819 | 0.5971343 | 1.3243439 | 0.9002595 |
| TG 53:2-FA16:0 | 5.3185278 | 4.2107109 | 4.6523746 | 4.9269062 | 6.2303314 | 3.685392 | 2.542874 | 2.1828606 | 3.8226441 | 7.4533654 |
| TG 53:2-FA17:0 | 1.1276637 | 2.8587178 | 1.54431 | 1.0754771 | 1.323073 | 1.1829786 | 0.7317666 | 0.6600461 | 1.2225519 | 2.1502887 |
| TG 53:2-FA18:1 | 5.9629887 | 8.3858975 | 6.216979 | 5.7340677 | 6.5604733 | 4.658409 | 2.8672317 | 2.2630417 | 4.3330643 | 8.7923094 |
| TG 53:2-FA18:2 | 1.6908351 | 2.3279832 | 3.0129431 | 1.9859103 | 2.2744602 | 1.8758906 | 1.8119645 | 1.2110577 | 2.3633812 | 1.4734957 |
| TG 53:3-FA16:0 | 17.284598 | 5.3237064 | 8.2225 | 14.602336 | 16.794678 | 9.304698 | 7.6059697 | 5.9487566 | 7.5314741 | 17.106466 |
| TG 53:3-FA17:0 | 3.208832 | 4.2140843 | 3.3026786 | 3.2515396 | 3.034224 | 2.8214378 | 1.7526909 | 1.6777456 | 2.6235677 | 3.9414352 |
| TG 53:3-FA18:2 | 7.9940151 | 5.8853162 | 7.4105931 | 7.4626818 | 7.222252 | 5.1646589 | 4.2619869 | 3.4767275 | 5.4222112 | 7.3093687 |
| TG 53:4-FA16:0 | 19.220529 | 2.4531045 | 6.6840598 | 17.14756 | 15.887418 | 8.6660385 | 10.259438 | 7.0937389 | 7.3325889 | 11.553586 |
| TG 53:4-FA17:0 | 2.569774 | 1.9971057 | 2.3873965 | 2.6422694 | 2.2239747 | 1.9900359 | 1.9637091 | 1.6534279 | 2.1240223 | 2.5300072 |
| TG 53:4-FA18:2 | 18.680593 | 7.9630895 | 11.245809 | 18.147084 | 15.3875 | 10.201647 | 10.191273 | 7.7426648 | 9.3949928 | 13.819696 |
| TG 53:4-FA18:3 | 0.6495479 | 0.4165915 | 0.629375 | 0.549457 | 0.5145782 | 0.4837868 | 0.465547 | 0.3615021 | 0.527381 | 0.4682046 |
| TG 53:4-FA20:4 | 0.0968208 | 0.1443208 | 0.1414927 | 0.0808969 | 0.0928656 | 0.1125823 | 0.1060256 | 0.0910089 | 0.0945049 | 0.1130491 |
| TG 53:5-FA20:4 | 0.1509951 | 0.1622791 | 0.215149 | 0.1189154 | 0.1161022 | 0.1379873 | 0.0934244 | 0.1314588 | 0.1169896 | 0.1762995 |
| TG 53:6-FA20:4 | 0.1724327 | 0.1378428 | 0.2633013 | 0.168674 | 0.1421201 | 0.1390531 | 0.1246512 | 0.151185 | 0.1285965 | 0.1582433 |
| TG 54:0-FA16:0 | 1.1493524 | 0.7092794 | 0.845747 | 1.0432742 | 0.7479309 | 1.0179924 | 1.1322308 | 1.0268387 | 1.2628038 | 1.0443827 |
| TG 54:0-FA18:0 | 0.2039207 | 0.1852787 | 0.2837192 | 0.1830644 | 0.1800029 | 0.1648427 | 0.3760469 | 0.2137111 | 0.2846658 | 0.1697312 |
| TG 54:1-FA16:0 | 1.616058 | 1.0401875 | 3.1278605 | 1.6372369 | 1.1075205 | 0.7323029 | 1.2337184 | 0.955373 | 1.7456875 | 1.0779425 |
| TG 54:1-FA18:0 | 0.9960683 | 1.6120721 | 1.5859 | 1.0027806 | 1.2880305 | 0.8675356 | 1.0004515 | 0.7732536 | 1.5198163 | 1.0657675 |
| TG 54:1-FA18:1 | 1.1361895 | 1.4828711 | 2.3337895 | 1.2953693 | 0.9787507 | 0.8207831 | 0.8981808 | 0.6803633 | 1.3780619 | 1.0708117 |
| TG 54:1-FA20:0 | 14.194076 | 7.2855118 | 9.9333831 | 12.283018 | 11.208704 | 10.38505 | 7.2662403 | 6.7150816 | 6.8149302 | 13.06548 |
| TG 54:1-FA20:1 | 0.6071943 | 0.4576811 | 0.8178075 | 0.4044375 | 0.6036383 | 0.2901206 | 0.5567058 | 0.321768 | 0.6841791 | 0.2426215 |
| TG 54:2-FA16:0 | 12.225185 | 6.1722617 | 12.271117 | 10.817681 | 11.211388 | 5.0760385 | 5.3270479 | 4.1592731 | 7.0826858 | 9.687061 |
| TG 54:2-FA18:0 | 3.2593531 | 8.6873 | 4.8095988 | 4.4070098 | 4.081495 | 4.071097 | 2.578342 | 2.6939302 | 4.2810834 | 5.6446397 |
| TG 54:2-FA18:1 | 12.22165 | 14.875966 | 14.217257 | 12.982229 | 12.661759 | 8.1006557 | 5.5860476 | 4.6653785 | 9.1367603 | 14.109662 |
| TG 54:2-FA18:2 | 1.8431459 | 1.701861 | 3.9594703 | 2.3015066 | 1.3672126 | 1.1576146 | 1.7652506 | 1.5723547 | 2.498757 | 1.416199 |
| TG 54:2-FA20:0 | 21.387194 | 5.4704476 | 12.818606 | 18.693617 | 13.790443 | 13.176905 | 14.169308 | 11.825418 | 9.485324 | 11.707226 |
| TG 54:2-FA20:1 | 9.6797972 | 6.3198372 | 10.105694 | 7.8519305 | 8.7466237 | 4.7514415 | 4.1458178 | 3.3927828 | 6.0037244 | 7.6840873 |
| TG 54:2-FA20:2 | 0.0791538 | 0.0779797 | 0.094847 | 0.0897675 | 0.1031214 | 0.0751323 | 0.0716613 | 0.1345632 | 0.1180061 | 0.0547477 |
| TG 54:3-FA16:0 | 20.669429 | 9.2656615 | 16.19026 | 16.944247 | 15.609173 | 8.2299069 | 10.172962 | 7.3673748 | 10.350441 | 12.367977 |
| TG 54:3-FA16:1 | 1.0153726 | 1.0243691 | 1.2372643 | 0.7355545 | 1.0034918 | 0.6545711 | 0.4821367 | 0.4154132 | 0.8846186 | 1.1418837 |
| TG 54:3-FA18:0 | 11.494742 | 17.305797 | 17.509318 | 15.201138 | 12.354841 | 12.448251 | 7.8214258 | 8.5707281 | 11.096537 | 16.327411 |
| TG 54:3-FA18:1 | 32.819714 | 184.28887 | 43.237543 | 56.463766 | 36.82944 | 46.347719 | 15.105505 | 15.211376 | 25.836956 | 94.108094 |
| TG 54:3-FA18:2 | 22.825376 | 17.610447 | 26.054251 | 23.945148 | 19.630115 | 14.497941 | 12.767695 | 11.102025 | 16.065001 | 18.632807 |
| TG 54:3-FA18:3 | 0.0668652 | 0.0804363 | 0.1898828 | 0.1471322 | 0.0926299 | 0.0632462 | 0.1269908 | 0.0822185 | 0.1569287 | 0.097694 |
| TG 54:3-FA20:1 | 17.838339 | 7.8677587 | 16.69769 | 14.274079 | 13.04956 | 7.6799101 | 9.184331 | 6.6736911 | 10.074825 | 9.2995223 |
| TG 54:3-FA20:2 | 1.9745251 | 3.6609291 | 3.0561036 | 1.2204351 | 1.4339753 | 1.6708669 | 0.8004731 | 0.9647073 | 1.372158 | 2.8423 |
| TG 54:3-FA20:3 | 0.1226568 | 0.146679 | 0.1792068 | 0.0864432 | 0.1145 | 0.0633277 | 0.1060213 | 0.0946062 | 0.1412612 | 0.165948 |
| TG 54:4-FA16:0 | 5.3827175 | 5.859939 | 5.9641155 | 3.7862155 | 3.875265 | 4.5775899 | 3.2382468 | 3.0983326 | 3.6206061 | 5.5682667 |
| TG 54:4-FA16:1 | 1.6494845 | 1.5268975 | 1.585022 | 1.239682 | 1.3311601 | 0.9653195 | 0.7054699 | 0.5834414 | 1.0426438 | 1.5705605 |
| TG 54:4-FA18:0 | 12.617725 | 12.153319 | 18.269923 | 15.143984 | 12.480964 | 12.563349 | 9.4724818 | 10.663643 | 11.535538 | 14.905929 |
| TG 54:4-FA18:1 | 66.809049 | 204.34812 | 78.2769 | 135.94956 | 67.401406 | 87.688549 | 30.066292 | 28.350104 | 44.378459 | 146.24601 |
| TG 54:4-FA18:2 | 49.438253 | 108.02652 | 64.587267 | 86.953016 | 49.028126 | 56.524523 | 28.146583 | 29.422671 | 37.596002 | 88.329112 |
| TG 54:4-FA18:3 | 1.5702321 | 1.373901 | 1.7609458 | 1.5049541 | 1.2624941 | 1.2033384 | 1.0663075 | 1.0849555 | 1.4711891 | 1.3145045 |
| TG 54:4-FA20:1 | 1.7816731 | 1.1583197 | 2.1458937 | 1.2781627 | 1.52025 | 1.0546378 | 1.1222107 | 0.7587869 | 1.5707327 | 1.2821875 |
| TG 54:4-FA20:2 | 3.3208802 | 3.3456587 | 3.8363309 | 2.3795199 | 2.2066624 | 2.4912292 | 2.0126249 | 1.9869687 | 2.0774166 | 3.3133612 |
| TG 54:4-FA20:3 | 2.239067 | 4.451676 | 3.1750876 | 1.6091587 | 1.6670006 | 2.7074016 | 1.0735468 | 1.1637841 | 1.3362874 | 3.7982884 |
| TG 54:4-FA20:4 | 0.4331668 | 0.6591145 | 0.7753412 | 0.3531731 | 0.3579099 | 0.5207677 | 0.4419867 | 0.59629 | 0.5205449 | 0.5978301 |
| TG 54:4-FA22:1 | 0.1461164 | 0.3831198 | 0.1993109 | 0.3063438 | 0.1606063 | 0.1766911 | 0.125522 | 0.1027603 | 0.1231422 | 0.3317082 |
| TG 54:4-FA22:4 | 0.074536 | 0.0813867 | 0.2345241 | | 0.0956812 | 0.0829547 | 0.0744143 | | | 0.1310794 |
| TG 54:5-FA16:0 | 4.7430051 | 4.793812 | 5.7889723 | 3.6233809 | 3.312278 | 5.0060141 | 3.2269391 | 3.6036203 | 3.4001591 | 5.2162219 |
| TG 54:5-FA16:1 | 0.6520293 | 0.9812208 | 0.6770883 | 0.5668264 | 0.4798904 | 0.6651573 | 0.3193918 | 0.2817951 | 0.4010292 | 1.0164709 |
| TG 54:5-FA18:0 | 1.1206539 | 1.160306 | 1.5992636 | 1.0012825 | 0.8573877 | 1.0500579 | 1.0819614 | 1.0558293 | 1.3236576 | 0.8564287 |
| TG 54:5-FA18:1 | 49.132863 | 73.376607 | 60.634013 | 109.03741 | 43.513611 | 55.024643 | 28.954374 | 24.469843 | 32.514147 | 81.865646 |
| TG 54:5-FA18:2 | 77.775072 | 101.36628 | 94.857614 | 178.18765 | 66.78242 | 81.167564 | 45.874365 | 37.970754 | 49.961393 | 123.00723 |
| TG 54:5-FA18:3 | 4.7903383 | 9.4811678 | 5.8616903 | 6.8152574 | 4.2275198 | 6.2045859 | 3.0723961 | 2.7805895 | 4.0018467 | 8.1067715 |
| TG 54:5-FA20:2 | 0.4932892 | 0.6330879 | 0.463059 | 0.5155041 | 0.3840774 | 0.5244033 | 0.2930147 | 0.282756 | 0.3123009 | 0.6583465 |
| TG 54:5-FA20:3 | 4.2450382 | 4.3658788 | 4.579077 | 3.1576291 | 2.7961194 | 4.4109897 | 2.5088053 | 2.8711051 | 2.7357407 | 4.6543422 |
| TG 54:5-FA20:4 | 4.0348867 | 5.8203564 | 9.8803267 | 3.446961 | 3.3989538 | 5.3652634 | 2.4782176 | 3.2189614 | 3.3805672 | 7.1152484 |
| TG 54:5-FA20:5 | 0.1753756 | 0.3326637 | 0.4234766 | 0.1558244 | 0.197898 | 0.1836064 | 0.3228939 | 0.2607004 | 0.3145173 | 0.1553772 |
| TG 54:5-FA22:1 | 0.3550676 | 0.4624936 | 0.3995844 | 0.7186361 | 0.2712683 | 0.3484835 | 0.2126328 | 0.1812634 | 0.2290578 | 0.569816 |
| TG 54:5-FA22:4 | 0.122759 | 0.1046573 | 0.218345 | 0.0976408 | 0.0913823 | 0.1020538 | 0.0627134 | 0.0858272 | 0.1228986 | 0.2115222 |
| TG 54:5-FA22:5 | 0.5651498 | 0.6287319 | 0.6560205 | 0.3255742 | 0.3451252 | 0.4452573 | 0.5417784 | 0.4335443 | 0.542689 | 0.5057677 |
| TG 54:6-FA16:0 | 5.2026666 | 5.3839028 | 6.1156022 | 3.7548901 | 3.4209423 | 6.1139759 | 4.8286521 | 5.2999262 | 5.564777 | 5.8455516 |
| TG 54:6-FA16:1 | 0.594619 | 0.9590042 | 0.6756281 | 0.4298076 | 0.4675005 | 0.9052873 | 0.3086252 | 0.3764263 | 0.5124383 | 0.9214491 |
| TG 54:6-FA18:1 | 8.4592688 | 11.712008 | 8.9133954 | 9.8605864 | 6.7394686 | 11.283297 | 6.760039 | 5.6475114 | 7.822563 | 11.472308 |
| TG 54:6-FA18:2 | 54.476976 | 41.047431 | 73.444397 | 133.64708 | 39.103051 | 49.179985 | 40.817592 | 31.188335 | 34.759774 | 82.168215 |
| TG 54:6-FA18:3 | 7.8233194 | 10.339774 | 7.7460116 | 9.9148366 | 6.4001349 | 10.18123 | 6.2444333 | 4.9515372 | 6.2190122 | 9.9063547 |
| TG 54:6-FA20:3 | 0.596303 | 0.6746448 | 0.6292875 | 0.5174927 | 0.4428117 | 0.7236201 | 0.3045164 | 0.3646175 | 0.3428559 | 0.7844741 |
| TG 54:6-FA20:4 | 7.9455403 | 6.1266282 | 14.437307 | 6.9382839 | 5.7510325 | 9.3418635 | 6.8800085 | 8.1299534 | 7.2928845 | 9.3260412 |
| TG 54:6-FA20:5 | 6.5955865 | 7.6712711 | 5.4380815 | 4.1905094 | 3.7216168 | 8.4258418 | 6.0385088 | 6.5795417 | 8.5937119 | 7.8816584 |
| TG 54:6-FA22:5 | 0.565235 | 0.7080745 | 0.7601431 | 0.289024 | 0.4196427 | 0.6964413 | 0.3952685 | 0.4999061 | 0.6755072 | 0.8895939 |
| TG 54:6-FA22:6 | 2.8100895 | 4.6887907 | 3.2530068 | 2.1759469 | 3.0404141 | 3.4864916 | 4.8316347 | 4.1022502 | 4.4027643 | 4.6576408 |
| TG 54:7-FA16:1 | 0.6645832 | 1.2003398 | 0.7444573 | 0.4169 | 0.5229523 | 1.1054491 | 0.5422547 | 0.7126415 | 0.9614659 | 1.3574475 |
| TG 54:7-FA18:1 | 0.9791063 | 1.38759 | 0.8017178 | 0.6990874 | 0.7588038 | 1.372599 | 0.8276881 | 0.6260453 | 1.0689312 | 1.4472487 |
| TG 54:7-FA18:2 | 10.476725 | 7.2147484 | 8.6980643 | 9.3955076 | 6.5168746 | 12.064637 | 11.572689 | 8.5474857 | 9.9847871 | 8.2828207 |
| TG 54:7-FA18:3 | 5.091929 | 3.7184071 | 4.4231061 | 5.1993361 | 3.6072713 | 6.1725275 | 5.5878428 | 3.553433 | 4.2681534 | 4.3807531 |
| TG 54:7-FA20:4 | 0.923502 | 0.8510052 | 1.3937621 | 0.7015802 | 0.7073967 | 1.2670384 | 0.6551153 | 0.7827062 | 0.8533401 | 1.0523091 |
| TG 54:7-FA20:5 | 15.689321 | 9.2390035 | 8.3679805 | 9.8157946 | 7.1338787 | 16.819412 | 19.403987 | 17.710308 | 19.381178 | 10.843663 |
| TG 54:7-FA22:5 | 0.3898452 | 0.3301954 | 0.4741761 | 0.2906896 | 0.1977486 | 0.3930694 | 0.3026812 | 0.3342857 | 0.4906248 | 0.4705547 |
| TG 54:7-FA22:6 | 2.9412202 | 5.7026083 | 2.7139558 | 1.6971106 | 2.1736408 | 4.3144537 | 2.4550099 | 3.4225617 | 4.4601094 | 7.1079182 |
| TG 54:8-FA18:2 | 1.2026018 | 0.9001796 | 0.9253274 | 1.0245284 | 0.6833828 | 1.4824816 | 1.2317513 | 1.1076071 | 1.4010036 | 1.1596195 |
| TG 54:8-FA18:3 | 0.9125025 | 0.5089961 | 0.6210165 | 0.566435 | 0.5242764 | 1.1799095 | 0.9830913 | 0.7513419 | 0.9520234 | 0.5710233 |
| TG 54:8-FA20:4 | 0.1389829 | 0.0987761 | 0.1412895 | 0.0989314 | 0.0888604 | 0.10703 | 0.1113486 | 0.1039811 | 0.1336494 | 0.1152366 |
| TG 54:8-FA20:5 | 1.6835158 | 1.2966606 | 1.0383133 | 1.1385534 | 0.9893126 | 2.1251267 | 1.5706018 | 1.6463666 | 2.2921794 | 1.7405891 |
| TG 54:8-FA22:6 | 1.6958192 | 2.1056517 | 1.7119025 | 1.3317854 | 0.9952366 | 2.073546 | 1.7361597 | 2.2974298 | 2.5686323 | 3.5138573 |
| TG 55:1-FA16:0 | 11.985884 | 11.351112 | 7.5431999 | 9.164662 | 6.4381221 | 13.386236 | 16.003512 | 17.003833 | 17.428036 | 14.040206 |
| TG 55:1-FA18:1 | 1.772846 | 3.3761952 | 1.692457 | 1.549361 | 1.3846252 | 2.5576971 | 1.7899809 | 1.5161226 | 2.3585507 | 2.580526 |
| TG 55:2-FA18:1 | 1.5625577 | 1.8427527 | 2.1669996 | 1.6636767 | 1.6298574 | 1.3157373 | 1.0801886 | 0.756931 | 1.6130821 | 1.4447385 |
| TG 55:2-FA18:2 | 2.7573084 | 3.010312 | 2.1419693 | 2.1964132 | 1.9760088 | 3.2526393 | 3.3581441 | 2.7101047 | 3.4311132 | 2.7484116 |
| TG 55:3-FA18:1 | 3.0319306 | 4.0181895 | 3.2160746 | 4.9341415 | 3.5960141 | 2.0292507 | 1.4242871 | 1.0982899 | 2.3744045 | 4.8488669 |
| TG 55:3-FA18:2 | 2.6047316 | 2.4241758 | 3.6018097 | 2.8238709 | 2.5936621 | 1.9437678 | 2.0128876 | 1.3310783 | 2.6034387 | 1.9810247 |
| TG 55:4-FA18:1 | 6.3730674 | 4.4091635 | 4.9976414 | 12.670491 | 7.0343263 | 3.7176087 | 2.0622979 | 1.6118904 | 2.9777705 | 9.3119668 |
| TG 55:4-FA18:2 | 4.3925807 | 2.9511537 | 4.5417128 | 7.2534452 | 4.4540404 | 2.583523 | 2.2857002 | 1.9818949 | 3.0494987 | 4.6639166 |
| TG 55:5-FA18:1 | 6.0635994 | 2.0682981 | 4.4689334 | 13.587629 | 6.0151006 | 2.9789839 | 2.4561594 | 1.6545558 | 2.6003203 | 6.8654251 |
| TG 55:5-FA18:2 | 8.4093259 | 2.5806949 | 6.7291793 | 19.229803 | 7.5113793 | 3.6852198 | 3.3356899 | 2.0984979 | 3.4732717 | 8.7563335 |
| TG 55:5-FA20:4 | 0.2698623 | 0.2187761 | 0.4756662 | 0.2876703 | 0.2548771 | 0.2610854 | 0.1928144 | 0.2287893 | 0.2360646 | 0.3639041 |
| TG 55:7-FA15:0 | 0.1950142 | 0.2589216 | 0.1469709 | 0.1801653 | 0.0914799 | 0.1578619 | 0.1600882 | 0.159395 | 0.2426498 | 0.2105549 |
| TG 55:7-FA22:6 | 0.8651161 | 1.5146913 | 0.8094771 | 0.6554949 | 0.5633901 | 0.7749584 | 0.7441138 | 0.7798398 | 1.1595325 | 1.3137932 |
| TG 56:1-FA16:0 | 1.6053275 | 0.4040616 | 1.4224951 | 1.4284581 | 0.9871831 | 0.8317609 | 1.4313223 | 1.2747588 | 1.5248454 | 1.243129 |
| TG 56:1-FA18:1 | 0.7798805 | 0.8511859 | 1.1755655 | 0.8583834 | 0.5754086 | 0.6080868 | 0.5677318 | 0.3943184 | 0.827681 | 0.7264288 |
| TG 56:10-FA18:2 | 2.0985675 | 1.7868047 | 2.7954954 | 2.4434142 | 2.1756658 | 1.525474 | 1.495372 | 1.0573196 | 1.9875164 | 1.4209349 |
| TG 56:2-FA16:0 | 1.2375164 | 0.5515216 | 2.6198029 | 1.1760987 | 0.8150949 | 0.4167057 | 0.7020791 | 0.4395321 | 1.0783986 | 0.5718921 |
| TG 56:2-FA18:0 | 0.9688168 | 0.8033405 | 1.3378648 | 1.1016261 | 1.0918091 | 0.5418894 | 0.7122821 | 0.4729324 | 0.9332472 | 0.6237173 |
| TG 56:2-FA20:0 | 4.0569991 | 3.5399992 | 4.3033478 | 6.8505747 | 3.6487857 | 2.9409767 | 2.0478823 | 1.9108358 | 2.4694491 | 4.1825619 |
| TG 56:2-FA20:1 | 2.0404791 | 1.2625549 | 2.6400593 | 1.4686756 | 1.8962891 | 0.8780679 | 1.0985722 | 0.5545914 | 1.4783435 | 0.8010517 |
| TG 56:3-FA16:0 | 0.8685676 | 0.3522645 | 2.1764449 | 0.9117486 | 0.5291865 | 0.3215187 | 0.5406975 | 0.4189841 | 0.8665774 | 0.4409977 |
| TG 56:3-FA18:0 | 1.8526984 | 1.3220453 | 2.3065065 | 1.8593845 | 2.0211286 | 1.0014426 | 1.1343413 | 0.8378398 | 1.4713408 | 1.0649977 |
| TG 56:3-FA18:1 | 6.3389076 | 7.7025637 | 8.1873739 | 10.19397 | 5.7809596 | 3.2058063 | 2.5563325 | 1.7626077 | 4.5220422 | 6.5965685 |
| TG 56:3-FA18:2 | 2.8977192 | 1.7819532 | 4.4499044 | 3.6983712 | 2.4375531 | 1.3397813 | 1.8843867 | 1.3679331 | 2.8100872 | 1.6177009 |
| TG 56:3-FA20:0 | 6.9828324 | 3.7430023 | 7.032396 | 15.053239 | 5.1763197 | 4.1551708 | 3.5852962 | 2.9823419 | 3.5720773 | 6.3705449 |
| TG 56:3-FA20:1 | 4.2666704 | 5.594468 | 5.6705014 | 5.1765392 | 4.0611273 | 2.4562741 | 2.0331056 | 1.3935768 | 3.201593 | 3.7607079 |
| TG 56:3-FA20:2 | 0.189712 | 0.2476521 | 0.2879046 | 0.1539378 | 0.1763879 | 0.1449998 | 0.1040864 | 0.1012137 | 0.1754072 | 0.1986526 |
| TG 56:4-FA16:0 | 0.4597227 | 0.3026011 | 0.9480736 | 0.43515 | 0.3280954 | 0.2688797 | 0.2119316 | 0.2527375 | 0.3275968 | 0.3460388 |
| TG 56:4-FA18:0 | 0.4822086 | 0.5732347 | 0.632315 | 0.5177534 | 0.4376078 | 0.3916621 | 0.4429047 | 0.4423657 | 0.4756645 | 0.5753313 |
| TG 56:4-FA18:1 | 9.9798616 | 11.023924 | 12.356659 | 13.926203 | 8.2441948 | 5.1741089 | 3.9845718 | 2.7244464 | 5.8093428 | 8.3239548 |
| TG 56:4-FA18:2 | 9.3698736 | 6.5342645 | 12.401547 | 15.010556 | 6.8044602 | 4.3490774 | 4.4564145 | 3.1213394 | 6.1090297 | 6.5042603 |
| TG 56:4-FA20:1 | 9.2085936 | 7.7089376 | 12.146546 | 12.200068 | 7.022541 | 4.5844626 | 3.7844283 | 2.6679943 | 5.3654821 | 6.4608278 |
| TG 56:4-FA20:2 | 0.6399904 | 2.6814872 | 0.8346819 | 0.5373963 | 0.5998813 | 0.692102 | 0.3499461 | 0.2874326 | 0.4992086 | 1.3013044 |
| TG 56:4-FA20:3 | 0.3566796 | 0.4674991 | 0.3869436 | 0.2993454 | 0.3163095 | 0.3194446 | 0.2600725 | 0.2212642 | 0.2216734 | 0.4537941 |
| TG 56:4-FA20:4 | 0.1306434 | 0.2156942 | 0.2684216 | 0.0713189 | 0.1109671 | 0.1413474 | 0.1989063 | 0.1863584 | 0.1096767 | 0.1233152 |
| TG 56:4-FA22:4 | 0.042223 | 0.023489 | 0.0535058 | 0.0735336 | 0.0666587 | 0.0383517 | 0.0552352 | 0.0473116 | 0.0749587 | 0.0303457 |
| TG 56:5-FA16:0 | 0.6862304 | 0.6504839 | 1.5056035 | 0.566482 | 0.4183962 | 0.5502818 | 0.4624041 | 0.4036591 | 0.5260626 | 0.8900516 |
| TG 56:5-FA18:0 | 0.7925047 | 0.9227338 | 0.8766011 | 0.7003672 | 0.5950562 | 0.9214197 | 0.7165283 | 0.6208238 | 0.7556983 | 1.0149763 |
| TG 56:5-FA18:1 | 2.9431145 | 7.6634489 | 3.3889469 | 2.5358745 | 2.3918964 | 3.1312465 | 1.3779185 | 1.2798159 | 1.725649 | 4.8051946 |
| TG 56:5-FA18:2 | 12.037402 | 7.9948286 | 15.035152 | 17.453492 | 8.1216459 | 5.2720888 | 5.5359871 | 3.4820787 | 6.1674398 | 7.8976623 |
| TG 56:5-FA20:1 | 6.8958683 | 3.9813884 | 9.8790765 | 9.0861901 | 4.8461084 | 3.0344686 | 3.5872901 | 2.2899465 | 3.9982693 | 3.8140944 |
| TG 56:5-FA20:2 | 1.2861763 | 3.2472608 | 1.2456492 | 1.0710782 | 1.0425892 | 1.2860054 | 0.6275956 | 0.5128759 | 0.7632814 | 1.9160283 |
| TG 56:5-FA20:3 | 0.9172697 | 3.5474895 | 0.9192529 | 0.7886125 | 0.7332004 | 1.4098092 | 0.4989896 | 0.5344915 | 0.5073911 | 1.8423676 |
| TG 56:5-FA20:4 | 1.4332458 | 1.7146645 | 1.7350408 | 1.1110639 | 1.2088776 | 1.7321703 | 1.0476101 | 1.0845113 | 1.2210974 | 1.8083488 |
| TG 56:5-FA22:4 | 0.7308692 | 0.9226774 | 1.8036466 | 0.5436839 | 0.6437692 | 0.7581861 | 0.3859566 | 0.4729393 | 0.5738586 | 1.2050756 |
| TG 56:5-FA22:5 | 0.2015121 | 0.2667668 | 0.2854471 | 0.0933575 | 0.1725375 | 0.1739907 | 0.1225894 | 0.2493275 | 0.3651387 | 0.2268185 |
| TG 56:6-FA16:0 | 3.4613036 | 4.4042039 | 4.2832694 | 2.2375756 | 1.6784443 | 3.4172603 | 2.4324795 | 3.0317337 | 3.615643 | 4.0865488 |
| TG 56:6-FA18:0 | 0.8885323 | 1.0923573 | 0.9890578 | 0.7516937 | 0.6452656 | 1.0867573 | 0.8850046 | 0.9880463 | 0.8717978 | 1.0112817 |
| TG 56:6-FA18:1 | 3.7766052 | 8.0391856 | 3.7948818 | 2.6728948 | 2.4714614 | 5.0526011 | 2.3180525 | 2.5666434 | 3.1107851 | 5.900193 |
| TG 56:6-FA18:2 | 3.7423415 | 5.1306671 | 3.9140648 | 3.5971202 | 2.6931907 | 3.9981367 | 2.4559808 | 2.2320193 | 2.5306123 | 3.9715095 |
| TG 56:6-FA18:3 | 0.8931425 | 0.6106358 | 0.9608954 | 0.7450631 | 0.5917072 | 0.5013023 | 0.6157796 | 0.378274 | 0.6447391 | 0.551563 |
| TG 56:6-FA20:2 | 1.0267291 | 1.4180455 | 0.8557055 | 0.825594 | 0.7729226 | 0.9621205 | 0.5872529 | 0.5501347 | 0.6346446 | 1.0149942 |
| TG 56:6-FA20:3 | 1.4916871 | 4.1681356 | 1.3094256 | 1.2509107 | 1.376673 | 2.3331937 | 0.8366843 | 0.8594364 | 0.9026444 | 2.6368616 |
| TG 56:6-FA20:4 | 2.9365472 | 5.2750639 | 4.2368224 | 2.7261381 | 2.4095983 | 3.9899925 | 2.4151102 | 2.4944971 | 2.1321881 | 4.7235272 |
| TG 56:6-FA20:5 | 0.7372065 | 0.7571044 | 0.6769615 | 0.5581847 | 0.5033641 | 0.6488954 | 0.6830036 | 0.5831559 | 0.8276774 | 0.553947 |
| TG 56:6-FA22:4 | 1.5758439 | 0.9830193 | 2.621979 | 1.3207136 | 0.9928337 | 1.3041008 | 1.0598577 | 1.2332633 | 1.2906129 | 1.6135338 |
| TG 56:6-FA22:5 | 4.392556 | 7.0899398 | 5.2886666 | 2.4241936 | 2.2516055 | 4.6920814 | 3.053634 | 3.3950957 | 4.7173427 | 6.1455251 |
| TG 56:6-FA22:6 | 0.7467765 | 1.3375705 | 0.8842041 | 0.6456339 | 0.5448526 | 0.8383656 | 0.9900179 | 1.2243412 | 1.3473864 | 0.7704392 |
| TG 56:7-FA16:0 | 8.4372408 | 13.666753 | 6.726199 | 5.336389 | 4.3435003 | 9.9295654 | 7.5604017 | 8.8260855 | 10.630731 | 13.423502 |
| TG 56:7-FA16:1 | 0.2459189 | 0.5703723 | 0.3226427 | 0.1593889 | 0.1617147 | 0.3123023 | 0.1644599 | 0.1480135 | 0.2411271 | 0.4971319 |
| TG 56:7-FA18:0 | 0.3703799 | 0.5282138 | 0.421635 | 0.2842657 | 0.3424564 | 0.4189773 | 0.5518074 | 0.4293295 | 0.6361838 | 0.4086342 |
| TG 56:7-FA18:1 | 5.3292084 | 12.568113 | 4.9873512 | 4.0777142 | 3.4849841 | 7.4095063 | 4.5418122 | 5.5750149 | 6.7397197 | 11.341416 |
| TG 56:7-FA18:2 | 6.1604503 | 5.9731691 | 4.5076686 | 4.4439819 | 3.388059 | 6.645437 | 5.386705 | 5.2719903 | 5.8680159 | 5.7816927 |
| TG 56:7-FA18:3 | 0.5557767 | 0.4476897 | 0.3594393 | 0.3499501 | 0.2723238 | 0.4839814 | 0.3829251 | 0.3338618 | 0.4225756 | 0.3931433 |
| TG 56:7-FA20:3 | 1.3978676 | 1.6424278 | 0.8556819 | 1.1227105 | 0.8723537 | 1.599319 | 0.8768386 | 0.8181572 | 0.7137558 | 1.5056134 |
| TG 56:7-FA20:4 | 3.0032954 | 4.880848 | 4.3022199 | 3.1473891 | 2.6305747 | 4.3693575 | 2.2616731 | 2.4769081 | 2.2967047 | 5.3087266 |
| TG 56:7-FA20:5 | 2.3926594 | 6.5482441 | 2.6159518 | 1.8851647 | 1.7595492 | 3.6857045 | 2.1918819 | 2.3817998 | 3.0737869 | 3.8932071 |
| TG 56:7-FA22:4 | 0.2237968 | 0.2561369 | 0.3116775 | 0.2288011 | 0.2137181 | 0.2535707 | 0.1920579 | 0.1739132 | 0.1999277 | 0.2977592 |
| TG 56:7-FA22:5 | 8.7398492 | 7.9146475 | 8.0152869 | 5.4549508 | 4.0041084 | 8.7185772 | 8.3398582 | 9.135769 | 10.211372 | 7.8991169 |
| TG 56:7-FA22:6 | 29.775174 | 61.786093 | 25.318778 | 21.264002 | 18.539068 | 37.592969 | 27.462515 | 33.996414 | 40.882104 | 61.851952 |
| TG 56:8-FA16:0 | 12.609236 | 11.993414 | 7.8116981 | 9.7363588 | 6.5313522 | 13.836611 | 16.907793 | 17.815811 | 18.526628 | 15.235317 |
| TG 56:8-FA16:1 | 0.7824397 | 2.1787695 | 0.7378088 | 0.6298401 | 0.5348103 | 1.2545158 | 0.5650419 | 0.748705 | 1.1537003 | 1.9987667 |
| TG 56:8-FA18:1 | 1.8627595 | 3.6926794 | 1.7367745 | 1.4901331 | 1.4550792 | 2.7252813 | 1.9700021 | 1.6374049 | 2.3748756 | 2.8772538 |
| TG 56:8-FA18:2 | 10.629407 | 11.071816 | 7.3308471 | 8.4981206 | 5.622619 | 12.117656 | 14.062816 | 14.436338 | 14.491338 | 13.21168 |
| TG 56:8-FA18:3 | 0.6229203 | 0.4971111 | 0.4707572 | 0.4801542 | 0.3862951 | 0.5993351 | 0.594416 | 0.5729257 | 0.6426395 | 0.5606146 |
| TG 56:8-FA20:4 | 2.1759817 | 2.1589263 | 2.7164772 | 2.6648445 | 1.5492202 | 2.6190226 | 2.1765619 | 2.05117 | 1.7662002 | 3.3486512 |
| TG 56:8-FA20:5 | 4.6735972 | 7.7382871 | 3.491882 | 3.8154731 | 2.8156342 | 6.4865852 | 5.0718435 | 4.5659686 | 5.3254134 | 5.9533181 |
| TG 56:8-FA22:5 | 1.7551594 | 1.4907639 | 1.2270485 | 1.2694301 | 0.9494264 | 1.7240079 | 1.6622111 | 1.8093872 | 1.9376101 | 1.9976429 |
| TG 56:8-FA22:6 | 63.853732 | 65.395593 | 39.277455 | 49.162551 | 32.61186 | 72.203484 | 86.245893 | 92.821154 | 92.962916 | 81.343388 |
| TG 56:9-FA18:3 | 0.5305573 | 0.5219917 | 0.4082774 | 0.3584168 | 0.3301804 | 0.75433 | 0.7491677 | 0.6107233 | 0.7807609 | 0.4960239 |
| TG 56:9-FA20:4 | 0.5032954 | 0.4609231 | 0.5746126 | 0.4494741 | 0.3226074 | 0.601354 | 0.5483907 | 0.4898031 | 0.5673155 | 0.457327 |
| TG 56:9-FA20:5 | 4.1437991 | 3.4082135 | 2.7151285 | 3.5725437 | 2.2052415 | 4.5787302 | 5.5348508 | 4.2781243 | 4.8175006 | 3.1801814 |
| TG 56:9-FA22:6 | 6.5491319 | 8.3111554 | 3.7400223 | 4.7039784 | 3.7364909 | 8.4070867 | 6.3349619 | 7.0960229 | 8.7980704 | 10.235329 |
| TG 57:10-FA22:6 | 0.5110137 | 0.5777839 | 0.4875674 | 0.5406665 | 0.327727 | 0.5320371 | 0.4393501 | 0.4596547 | 0.433532 | 0.5620103 |
| TG 57:2-FA18:1 | 2.9251516 | 8.2252034 | 2.0409277 | 2.4302746 | 2.0894928 | 4.2865151 | 2.9255469 | 2.9723439 | 3.3952997 | 5.6590019 |
| TG 57:3-FA18:2 | 3.8714702 | 5.2758119 | 2.5995841 | 3.4569396 | 2.3986648 | 4.397532 | 5.2134488 | 4.5317672 | 4.4677483 | 4.5090992 |
| TG 58:10-FA18:2 | 4.4221975 | 5.6416251 | 2.6529804 | 3.7763936 | 2.730768 | 4.8201172 | 5.5975298 | 4.9167399 | 4.8539765 | 4.8892319 |
| TG 58:10-FA20:4 | 0.3602954 | 0.5947944 | 1.2955617 | 0.4794791 | 0.27571 | 0.6648903 | 0.4857424 | 0.6214409 | 0.672089 | 0.6134025 |
| TG 58:10-FA20:5 | 0.4589477 | 0.5085492 | 0.5842055 | 0.3804132 | 0.2307909 | 0.6864688 | 0.4934039 | 0.5384777 | 0.7391065 | 0.4101342 |
| TG 58:10-FA22:5 | 0.6519632 | 0.5635677 | 0.3814518 | 0.683555 | 0.3717438 | 0.5826219 | 0.6926043 | 0.530651 | 0.7008108 | 0.597213 |
| TG 58:10-FA22:6 | 11.012601 | 16.2022 | 5.70975 | 9.8273411 | 5.9107265 | 13.594511 | 15.939093 | 13.953461 | 13.221581 | 14.343906 |
| TG 58:2-FA18:1 | 0.5655793 | 0.4087042 | 0.7088217 | 0.6494702 | 0.4428247 | 0.3774837 | 0.4305713 | 0.2859433 | 0.5298827 | 0.6588888 |
| TG 58:3-FA18:1 | 0.9520621 | 0.6824714 | 1.5479557 | 1.2111454 | 0.698485 | 0.4473456 | 0.5435783 | 0.3450993 | 0.8807334 | 0.5083378 |
| TG 58:5-FA18:1 | 0.3805348 | 0.3248975 | 0.3415479 | 0.2271275 | 0.2073949 | 0.1614809 | 0.1610215 | 0.1350554 | 0.1802437 | 0.2647135 |
| TG 58:6-FA16:0 | 0.4071606 | 0.2420382 | 0.3581268 | 0.2729804 | 0.2389375 | 0.1984817 | 0.2698546 | 0.2333059 | 0.3179598 | 0.3145039 |
| TG 58:6-FA18:0 | 0.1791477 | 0.2858965 | 0.1860051 | 0.1052963 | 0.1125213 | 0.1475264 | 0.1170385 | 0.1415364 | 0.2051691 | 0.2322429 |
| TG 58:6-FA18:1 | 0.3726696 | 0.83848 | 0.5269109 | 0.3081526 | 0.2996956 | 0.4020197 | 0.2448578 | 0.2224409 | 0.2897427 | 0.6023244 |
| TG 58:6-FA20:4 | 0.2927877 | 0.2218578 | 0.477532 | 0.3354714 | 0.1846971 | 0.1507009 | 0.2323421 | 0.2182915 | 0.268817 | 0.2415567 |
| TG 58:6-FA22:4 | 0.2727753 | 0.4974058 | 0.3550663 | 0.2120721 | 0.1635286 | 0.253629 | 0.1731682 | 0.1662906 | 0.182972 | 0.3915851 |
| TG 58:6-FA22:5 | 0.5336977 | 0.5273782 | 0.4034488 | 0.3419245 | 0.3287864 | 0.2864218 | 0.2890679 | 0.2892388 | 0.3719455 | 0.3921564 |
| TG 58:7-FA16:0 | 0.9316013 | 0.8096488 | 0.9028944 | 0.7159001 | 0.5317623 | 0.5723475 | 0.6625542 | 0.631401 | 0.8145158 | 0.9524403 |
| TG 58:7-FA18:0 | 0.4920296 | 1.0502944 | 0.4935149 | 0.3798368 | 0.2978464 | 0.6502011 | 0.558453 | 0.5719936 | 0.6987368 | 0.6030616 |
| TG 58:7-FA18:1 | 0.9285554 | 3.5404188 | 1.0927322 | 0.5467059 | 0.641561 | 1.313529 | 0.6154054 | 0.7452188 | 0.9385 | 1.9475797 |
| TG 58:7-FA18:2 | 0.6114118 | 0.7149293 | 0.6139316 | 0.479611 | 0.4207244 | 0.5434133 | 0.4301695 | 0.4599952 | 0.5650969 | 0.6267139 |
| TG 58:7-FA20:4 | 0.3659079 | 0.3614029 | 0.5435331 | 0.3938844 | 0.2543175 | 0.2154777 | 0.246953 | 0.227272 | 0.2957005 | 0.3105748 |
| TG 58:7-FA22:4 | 0.4310534 | 0.6245193 | 0.514551 | 0.420332 | 0.3323576 | 0.4195333 | 0.28904 | 0.2698005 | 0.3208274 | 0.6909426 |
| TG 58:7-FA22:5 | 0.9728607 | 3.2198796 | 0.9913563 | 0.6138967 | 0.6658026 | 1.3302385 | 0.7151471 | 0.7964869 | 1.038016 | 1.7615522 |
| TG 58:7-FA22:6 | 2.4435418 | 3.1266005 | 1.6674724 | 1.876611 | 1.8109162 | 1.9428412 | 2.0422004 | 1.9271718 | 2.5242041 | 2.7517185 |
| TG 58:8-FA18:1 | 2.386662 | 11.574439 | 1.7922929 | 1.7035223 | 1.5438518 | 3.9614375 | 1.8338699 | 1.9386952 | 2.7161829 | 6.4889086 |
| TG 58:8-FA18:2 | 1.7110386 | 2.9330888 | 1.5741988 | 1.2333453 | 1.007014 | 1.9275435 | 1.7638803 | 1.7630286 | 1.8781141 | 2.1622891 |
| TG 58:8-FA20:3 | 0.1566683 | 0.2303862 | 0.1957884 | 0.1229247 | 0.0933209 | 0.177207 | 0.1291211 | 0.1132496 | 0.1180484 | 0.1723326 |
| TG 58:8-FA20:4 | 0.1964682 | 0.260833 | 0.4923346 | 0.2304887 | 0.1745199 | 0.2623375 | 0.1862672 | 0.2033414 | 0.2448728 | 0.258092 |
| TG 58:8-FA22:5 | 1.8470517 | 4.4285245 | 1.5102366 | 1.302568 | 1.1322212 | 2.1241856 | 1.5099599 | 1.5539768 | 1.7807851 | 2.6809887 |
| TG 58:8-FA22:6 | 7.7558278 | 34.834972 | 6.1121957 | 6.3170695 | 5.8859621 | 12.047176 | 7.3777254 | 9.0206954 | 10.340549 | 20.488137 |
| TG 58:9-FA18:1 | 2.9459309 | 8.5754168 | 1.9447587 | 2.3993738 | 2.1042849 | 4.2737825 | 3.1596896 | 3.0469724 | 3.6260862 | 5.9095834 |
| TG 58:9-FA18:2 | 2.7295295 | 6.9428597 | 1.4298937 | 1.9884105 | 1.5960834 | 3.812873 | 2.9875529 | 2.8500466 | 3.1558437 | 4.6416031 |
| TG 58:9-FA20:4 | 0.2882595 | 0.3583856 | 0.8170358 | 0.2368924 | 0.1536211 | 0.394354 | 0.2468739 | 0.2884658 | 0.3031318 | 0.3852364 |
| TG 58:9-FA22:5 | 1.4641135 | 1.6897693 | 0.9048968 | 1.2096052 | 0.6884652 | 1.4583969 | 1.5246161 | 1.3255814 | 1.3715082 | 1.4596629 |
| TG 58:9-FA22:6 | 14.145824 | 41.337822 | 7.9283992 | 11.781143 | 9.2052605 | 21.497229 | 14.714832 | 15.219782 | 16.283505 | 30.001944 |
| TG 60:10-FA22:5 | 0.2012064 | 0.2397379 | 0.2211086 | 0.1585322 | 0.0877954 | 0.1869736 | 0.1663043 | 0.2098471 | 0.2210949 | 0.1636177 |
| TG 60:10-FA22:6 | 0.9106939 | 1.8474356 | 0.7233486 | 0.8416592 | 0.4869296 | 1.0420907 | 0.8955081 | 0.9963635 | 0.9637457 | 1.2472153 |
| TG 60:11-FA22:5 | 0.270961 | 0.2592851 | 0.3568713 | 0.2046883 | 0.1461545 | 0.2956254 | 0.2537908 | 0.3353681 | 0.4811203 | 0.2676213 |
| TG 60:11-FA22:6 | 1.0093512 | 1.9912373 | 1.1997479 | 0.894561 | 0.5884267 | 1.5320034 | 1.2230775 | 1.4593613 | 1.6182587 | 1.4921142 |
| TG 60:12-FA22:6 | 1.657596 | 2.8018163 | 1.7022288 | 1.5617667 | 0.7522003 | 2.7052629 | 3.0908736 | 3.7272301 | 4.3243699 | 2.497459 |

### REFERENCES

1. Schoeler, M. & Caesar, R. Dietary lipids, gut microbiota and lipid metabolism. Rev. Endocr. Metab. Disord. 20, 461-472 (2019).
2. Wahlström, A., Sayin, S. I., Marschall, H.-U. & Bäckhed, F. Intestinal Crosstalk between Bile Acids and Microbiota and Its Impact on Host Metabolism. Cell Metab. 24, 41-50 (2016).
3. Sharon, G., Sampson, T. R., Geschwind, D. H. & Mazmanian, S. K. The Central Nervous System and the Gut Microbiome. Cell (2016). doi: 10.1016/j.cell.2016.10.027
4. Fung, T. C., Olson, C. A. & Hsiao, E. Y. Interactions between the microbiota, immune and nervous systems in health and disease. Nature Neuroscience (2017). doi: 10.1038/nn.4476
5. Rabot, S. et al. Germ-free C57BL/6J mice are resistant to high-fat-diet-induced insulin resistance and have altered cholesterol metabolism. FASEB J. 24, 4948-4959 (2010).
6. Wikoff, W. R. et al. Metabolomics analysis reveals large effects of gut microflora on mammalian blood metabolites. Proc. Natl. Acad. Sci. 106, 3698-3703 (2009).
7. Yano, J. M. et al. Indigenous bacteria from the gut microbiota regulate host serotonin biosynthesis. Cell (2015). doi: 10.1016/j.cell.2015.02.047
8. Ivanov, I. I. et al. Induction of Intestinal Th17 Cells by Segmented Filamentous Bacteria. Cell 139, 485-498 (2009).
9. Sokol, H. et al. Faecalibacterium prausnitzii is an anti-inflammatory commensal bacterium identified by gut microbiota analysis of Crohn disease patients. Proc. Natl. Acad. Sci. 105, 16731-16736 (2008).
10. Storelli, G. et al. Drosophila Perpetuates Nutritional Mutualism by Promoting the Fitness of Its Intestinal Symbiont Lactobacillus plantarum. Cell Metab. 27, 362-377.e8 (2018).
11. Kim, C. Y. et al. Human reference gut microbiome catalog including newly assembled genomes from under-represented Asian metagenomes. Genome Med. 13, 134 (2021).
12. Huang, S. et al. The day-to-day stability of the ruminal and fecal microbiota in lactating dairy cows. Microbiologyopen 9, (2020).
13. Maki, J. J. & Looft, T. Turicibacter bilis sp. nov., a novel bacterium isolated from the chicken eggshell and swine ileum. Int. J. Syst. Evol. Microbiol. 72, (2022).
14. Feng, Y. et al. Metagenome-assembled genomes and gene catalog from the chicken gut microbiome aid in deciphering antibiotic resistomes. Commun. Biol. 4, 1305 (2021).
15. Browne, H. P. et al. Culturing of ‘unculturable’ human microbiota reveals novel taxa and extensive sporulation. Nature (2016). doi: 10.1038/nature17645
16. Martínez, I. et al. The Gut Microbiota of Rural Papua New Guineans: Composition, Diversity Patterns, and Ecological Processes. Cell Rep. 11, 527-538 (2015).
17. Mo, J. et al. Structural and quantitative alterations of gut microbiota in experimental small bowel obstruction. PLOS One 16, e0255651 (2021).
18. Goodrich, J. K. et al. Genetic Determinants of the Gut Microbiome in UK Twins. Cell Host Microbe 19, 731-743 (2016).
19. Benson, A. K. et al. Individuality in gut microbiota composition is a complex polygenic trait shaped by multiple environmental and host genetic factors. Proc. Natl. Acad. Sci. 107, 18933-18938 (2010).
20. Org, E. et al. Genetic and environmental control of host-gut microbiota interactions. Genome Res. (2015). doi: 10.1101/gr.194118.115
21. Kemis, J. H. et al. Genetic determinants of gut microbiota composition and bile acid profiles in mice. PLOS Genet. (2019). doi: 10.1371/journal.pgen. 1008073
22. Santiago-Rodriguez, T. M. et al. Gut Microbiome of an 11th Century A.D. Pre-Columbian Andean Mummy. PLOS One 10, e0138135 (2015).
23. Wibowo, M. C. et al. Reconstruction of ancient microbial genomes from the human gut. Nature 594, 234-239 (2021).
24. Yatsunenko, T. et al. Human gut microbiome viewed across age and geography. Nature 486, 222-227 (2012).
25. Turnbaugh, P. J. et al. An obesity-associated gut microbiome with increased capacity for energy harvest. Nature (2006). doi: 10.1038/nature05414
26 Thaiss, C. A. et al. Persistent microbiome alterations modulate the rate of post-dieting weight regain. Nature (2016). doi: 10.1038/nature20796
27. Chadaideh, K. S. & Carmody, R. N. Host-microbial interactions in the metabolism of different dietary fats. Cell Metab. 33, 857-872 (2021).
28. Dhakal, S., McCormack, L. & Dey, M. Association of the Gut Microbiota with Weight-Loss Response within a Retail Weight-Management Program. Microorganisms 8, 1246 (2020).
29. Liu, W. et al. Diet- and Genetically-induced Obesity Produces Alterations in the Microbiome, Inflammation and Wnt Pathway in the Intestine of Apc+/1638N Mice: Comparisons and Contrasts. J. Cancer 7, 1780-1790 (2016).
30. Li, T.-T. et al. Polyunsaturated fatty acids from microalgae Spirulina platensis modulates lipid metabolism disorders and gut microbiota in high-fat diet rats. Food Chem. Toxicol. 131, 110558 (2019).
31. Velázquez, K. T. et al. Prolonged high-fat-diet feeding promotes non-alcoholic fatty liver disease and alters gut microbiota in mice. World J. Hepatol. 11, 619-637 (2019).
32. Jiao, N. et al. Gut microbiome may contribute to insulin resistance and systemic inflammation in obese rodents: a meta-analysis. Physiol. Genomics 50, 244-254 (2018).
33. Petersen, C. et al. T cell-mediated regulation of the microbiota protects against obesity. Science (80-.). 365, (2019).
34 Golloso-Gubat, M. J. et al. Gut Microbiota and Dietary Intake of Normal-Weight and Overweight Filipino Children. Microorganisms 8, 1015 (2020).
35. Chen, X. et al. Alteration of the gut microbiota associated with childhood obesity by 16S rRNA gene sequencing. PeerJ 8, e8317 (2020).
36. Bosshard, P. P., Zbinden, R. & Altwegg, M. Turicibacter sanguinis gen. nov., sp. nov., a novel anaerobic, Gram-positive bacterium. Int. J. Syst. Evol. Microbiol. (2002). doi: 10.1099/ijs.0.02056-0
37. Maki, J. J., Nielsen, D. W. & Looft, T. Complete Genome Sequence and Annotation for Turicibacter sanguinis MOL361 T (DSM 14220). Microbiol. Resour. Announc. 9, (2020).
38. Fung, T. C. et al. Intestinal serotonin and fluoxetine exposure modulate bacterial colonization in the gut. Nature Microbiology (2019). doi: 10.1038/s41564-019-0540-4
39. Auchtung, T. A. et al. Complete genome sequence of Turicibacter sp. strain H121, isolated from the feces of a contaminated germ-free mouse. Genome Announc. (2016). doi: 10.1128/genomeA.00114-16
40. Russell, D. W. The Enzymes, Regulation, and Genetics of Bile Acid Synthesis. Annu. Rev. Biochem. 72, 137-174 (2003).
41. Guo, C., Chen, W.-D. & Wang, Y.-D. TGR5, Not Only a Metabolic Regulator. Front. Physiol. 7, (2016).
42. Ridlon, J. M., Kang, D.-J. & Hylemon, P. B. Bile salt biotransformations by human intestinal bacteria. J. Lipid Res. 47, 241-259 (2006).
43. Makishima, M. et al. Vitamin D Receptor As an Intestinal Bile Acid Sensor. Science (80-.). 296, 1313-1316 (2002).
44. Lou, D. et al. The three-dimensional structure of Clostridium absonum 7α-hydroxysteroid dehydrogenase: new insights into the conserved arginines for NADP (H) recognition. Sci. Rep. 6, 22885 (2016).
45. Klaver, F. A. & van der Meer, R. The assumed assimilation of cholesterol by Lactobacilli and Bifidobacterium bifidum is due to their bile salt-deconjugating activity. Appl. Environ. Microbiol. 59, 1120-1124 (1993).
46. Yao, L. et al. A selective gut bacterial bile salt hydrolase alters host metabolism. Elife (2018). doi: 10.7554/elife.37182
47. Joyce, S. A. et al. Regulation of host weight gain and lipid metabolism by bacterial bile acid modification in the gut. Proc. Natl. Acad. Sci. 111, 7421-7426 (2014).
48. Whitaker, W. R., Shepherd, E. S. & Sonnenburg, J. L. Tunable Expression Tools Enable Single-Cell Strain Distinction in the Gut Microbiome. Cell 169, 538-546.e12 (2017).
49. Hylemon, P. B. & Sherrod, J. A. Multiple forms of 7-alpha-hydroxysteroid dehydrogenase in selected strains of Bacteroides fragilis. J. Bacteriol. 122, 418-424 (1975).
50. Sherrod, J. A. & Hylemon, P. B. Partial purification and characterization of NAD-dependent 7α-hydroxysteroid dehydrogenase from Bacteroides thetaiotaomicron. Biochim. Biophys. Acta-Lipids Lipid Metab. 486, 351-358 (1977).
51. Marion, S. et al. Biogeography of microbial bile acid transformations along the murine gut. J. Lipid Res. 61, 1450-1463 (2020).
52. Hofmann, A. F., Hagey, L. R. & Krasowski, M. D. Bile salts of vertebrates: structural variation and possible evolutionary significance. J. Lipid Res. 51, 226-246 (2010).
53. Jones, B. V., Begley, M., Hill, C., Gahan, C. G. M. & Marchesi, J. R. Functional and comparative metagenomic analysis of bile salt hydrolase activity in the human gut microbiome. Proc. Natl. Acad. Sci. 105, 13580-13585 (2008).
54. Begley, M., Hill, C. & Gahan, C. G. M. Bile Salt Hydrolase Activity in Probiotics. Appl. Environ. Microbiol. 72, 1729-1738 (2006).
55. Prete, R. et al. Beneficial bile acid metabolism from Lactobacillus plantarum of food origin. Sci. Rep. 10, 1165 (2020).
56. Tanaka, H., Hashiba, H., Kok, J. & Mierau, I. Bile Salt Hydrolase of Bifidobacterium longum-Biochemical and Genetic Characterization. Appl. Environ. Microbiol. 66, 2502-2512 (2000).
57. Kim, G.-B., Miyamoto, C. M., Meighen, E. A. & Lee, B. H. Cloning and Characterization of the Bile Salt Hydrolase Genes (bsh) from Bifidobacterium bifidum Strains. Appl. Environ. Microbiol. 70, 5603-5612 (2004).
58. Ma, D. et al. Ketogenic diet enhances neurovascular function with altered gut microbiome in young healthy mice. Sci. Rep. 8, 6670 (2018).
59. Gao, X. et al. Correlations between α-Linolenic Acid-Improved Multitissue Homeostasis and Gut Microbiota in Mice Fed a High-Fat Diet. mSystems 5, (2020).
60. Everard, A. et al. Microbiome of prebiotic-treated mice reveals novel targets involved in host response during obesity. ISME J. 8, 2116-2130 (2014).
61. Jung, M.-J. et al. Chronic Repression of mTOR Complex 2 Induces Changes in the Gut Microbiota of Diet-induced Obese Mice. Sci. Rep. 6, 30887 (2016).
62. Huang, J. et al. Supplementation of Bacillus sp. DU-106 reduces hypercholesterolemia and ameliorates gut dysbiosis in high-fat diet rats. Appl. Microbiol. Biotechnol. 105, 287-299 (2021).
63. Golloso-Gubat, M. J. et al. Gut microbiota and dietary intake of normal-weight and overweight filipino children. Microorganisms (2020). doi: 10.3390/microorganisms8071015
64. Caesar, R., Tremaroli, V., Kovatcheva-Datchary, P., Cani, P. D. & Bäckhed, F. Crosstalk between Gut Microbiota and Dietary Lipids Aggravates WAT Inflammation through TLR Signaling. Cell Metab. 22, 658-668 (2015).
65. Martinez-Guryn, K. et al. Small Intestine Microbiota Regulate Host Digestive and Absorptive Adaptive Responses to Dietary Lipids. Cell Host Microbe 23, 458-469.e5 (2018).
66. Song, Z. et al. Taxonomic profiling and populational patterns of bacterial bile salt hydrolase (BSH) genes based on worldwide human gut microbiome. Microbiome 7, 9 (2019).
67. Jones, M. L., Martoni, C. J. & Prakash, S. Cholesterol lowering and inhibition of sterol absorption by Lactobacillus reuteri NCIMB 30242: a randomized controlled trial. Eur. J. Clin. Nutr. 66, 1234-1241 (2012).
68. Russell, B. J. et al. Intestinal Transgene Delivery with Native &lt;em&gt;E. coli&lt;/em&gt; Chassis Allows Persistent Physiological Changes. bioRxiv 2021.11.11.468006 (2021). doi: 10.1101/2021.11.11.468006
69. Jin, M. et al. Analysis of the Gut Microflora in Patients With Parkinson's Disease. Front. Neurosci. 13, 1184 (2019).
70. Barandouzi, Z. A., Starkweather, A. R., Henderson, W. A., Gyamfi, A. & Cong, X. S. Altered Composition of Gut Microbiota in Depression: A Systematic Review. Front. Psychiatry 11, (2020).
71. Jackson, M. A. et al. Gut microbiota associations with common diseases and prescription medications in a population-based cohort. Nat. Commun. (2018). doi: 10.1038/s41467-018-05184-7
72. Ferguson, J. M. SSRI Antidepressant Medications. Prim. Care Companion J. Clin. Psychiatry 03, 22-27 (2001).
73. Cascade, E., Kalali, A. H. & Kennedy, S. H. Real-world data on SSRI antidepressant side effects. Psychiatry (2009).
74. Duncan, S. H. Growth requirements and fermentation products of Fusobacterium prausnitzii, and a proposal to reclassify it as Faecalibacterium prausnitzii gen. nov., comb. nov. Int. J. Syst. Evol. Microbiol. 52, 2141-2146 (2002).
75. Eren, A. M. et al. Community-led, integrated, reproducible multi-omics with anvi'o. Nat. Microbiol. 6, 3-6 (2021).
76. Yoon, S.-H., Ha, S., Lim, J., Kwon, S. & Chun, J. A large-scale evaluation of algorithms to calculate average nucleotide identity. Antonie Van Leeuwenhoek 110, 1281-1286 (2017).
77 Altschul, S. F. et al. Gapped BLAST and PSI-BLAST: A new generation of protein database search programs. Nucleic Acids Research (1997). doi: 10.1093/nar/25.17.3389
78. Schindelin, J. et al. Fiji: an open-source platform for biological-image analysis. Nat. Methods 9, 676-682 (2012).
79. Galarraga, M. et al. Adiposoft: automated software for the analysis of white adipose tissue cellularity in histological sections. J. Lipid Res. 53, 2791-2796 (2012).
80. Kolde, R. pheatmap: Pretty Heatmaps. (2019).
81. Team, R. C. R: A language and environment for statistical computing. (2020).

### INCORPORATION BY REFERENCE

All publications and patent applications mentioned herein are hereby incorporated by reference in their entirety as if each individual publication or patent application was specifically and individually indicated to be incorporated by reference. In case of conflict, the present application, including any definitions herein, will control.

### EQUIVALENTS

Those skilled in the art will recognize, or be able to ascertain using no more than routine experimentation, many equivalents to the specific embodiments of the invention described herein. Such equivalents are intended to be encompassed by the following claims.

1. A method of preventing or treating a metabolic disorder in a subject, comprising administering to the subject a composition comprising a bacterial strain that expresses a bile salt-regulating gene or bile acid-regulating gene.

2. The method of claim 1, wherein the bacterial strain is Turicibacter sanguinis, Escherichia coli, or Bacteroides thetaiotaomicron.

3. The method of claim 1, wherein the bacterial strain regulates the bile salt by glycine conjugation or taurine conjugation.

4. The method of claim 1, wherein the bile salt-regulating gene encodes a bile salt hydrolase (BSH).

5. The method of claim 4, wherein the BSH is any one of the bile salt hydrolases listed in Table 1.

6. The method of claim 1, wherein the bile acid-regulating gene encodes a 7-alpha hydroxysteroid dehydrogenase.

7. The method of claim 6, wherein the amino acid sequence of the 7-alpha hydroxysteroid dehydrogenase is at least 40% homologous to an amino acid sequence of a Clostridium absonum 7-alpha hydroxysteroid dehydrogenase.

8. The method of claim 2, wherein the bile salt or bile acid is any one of the bile salts or bile acids listed in Table 3.

9. The method of claim 1, wherein administration of the composition alters the subject's lipidome.

10. The method of claim 1, wherein administration of the composition decreases white adipose tissue weight in the subject.

11. The method of claim 1, wherein administration of the composition alters health-associated lipid biomarkers in the subject.

12. The method of claim 11, wherein administration of the composition decreases plasma triglycerides (TG) levels in the subject.

13. The method of claim 11, wherein administration of the composition decreases cholesterol levels and/or cholesterol ester (CE) levels in the subject.

14. The method of claim 1, wherein administration of the composition decreases abdominal fat pad mass in the subject.

15. The method of claim 1, wherein administration of the composition increases a bile acid in the subject.

16. The method of claim 2, wherein the T. sanguinis bacterial strain is any one of the T. sanguinis bacterial strains listed in Table 4.

17-22. (canceled)

23. A method of treating or preventing a metabolic disorder in a subject, comprising
(a) depleting the gut microbiota of the subject,
(b) administering a composition comprising a bacterial strain that expresses a bile salt-regulating gene or bile acid-regulating gene.

24-37. (canceled)

38. A bacterial strain comprising a bile salt-regulating gene or bile acid-regulating gene, wherein the bacterial strain expresses a bile salt-regulating gene or bile acid-regulating gene encoded by an exogenous nucleic acid.

39. A composition comprising the bacterial strain of claim 38.

40-53. (canceled)

54. A method of making the composition of claim 39, comprising transforming a bacterial strain with a gene expression construct encoding a bile salt-regulating gene or bile acid-regulating gene operably coupled to a promoter that promotes expression of the bile salt-regulating gene or bile acid-regulating gene in the bacterial strain

55. (canceled)


SEQUENCE LISTING (60 sequences)

SEQ ID NO: 1 - DNA, 990 bp, Turicibacter sanguinis
  source 1..990: mol_type = genomic DNA
atgtgtacaggacttagcttagttacaaaagataataagcatttatttggacgaaatttagatgtacccgctacatatggacaagctgttcatattgttccaagaaattatgattggttcaatattgtgagtgatgaaacatatacgtcaaagtatgcttgtattgggatgggaattgttgtggatcgtcttccccttctttttgatgcagttaatgaaaaaggattagcaggtgccgggttaaattttacgcattttgctaagtttaacgaaaaagctgttgatggaaaaacgaatatttcaggttcaactttcttatactgggcattaagtaatttctcaaatttagatgaattaaaagaagccttaaaacagttaattattacgaatattccagttaaagaagggcttcctgttgcaggacttcactggatgtttacagatttaagtggaaaaagtattgtcattgagtacatggaagatggaatgcacgtatacgacaatccagtaggagccttaacaaatgatccaactttcccttggcatttaacaaatctatgccaatatgtaacattagatacaaaaactccagcaccaaaacaatttggagaatatgttgctaaaccatttggtcatgggcttaatatgtgtggaatcccaggagatgcttctccagctgcacgatttgttcgaacagtctatttccgtgatacagttgtcgaagcagatgatgaagtcagtggtgtttcagcattcttccaagtgctaacaggtgttaccgtcataaaaggaagcgaaattgatccaaatggagatatgaattacaccgtctataaatcttgtatgtgccaagaatcaggaacatactactacacagattataaaaaccgtcgtattagtgccgtttgcttatcaaaagccgacctagatggaaaagaaatcatcagctttgaatatcaaggtaaacaagatattttattccaaaactaa

SEQ ID NO: 2 - DNA, 990 bp, Turicibacter sanguinis
  source 1..990: mol_type = genomic DNA
atgtgtacagggttaagtttaactactaaagatggaaaacacttctttggtagaaatttagatgttcccgcttcatacggacaatctgttcatatcattccaagaaatttcaattggctaaatgtcgtaaatggtgaaacattagcttctaaatatgcgtgtattggaatgggaatcgttgtagataatcatccacttctttttgatgcagtcaatgaaaaaggattagctggtggtggattaaactttacgcactttgctaaatttaatgaaaatgccatcgatggaaaaataaatatctccgcatctgactttgtatactgggcacttagtaactttgcggatttaaatgagttacgtcaagcgttagaaaatttagtactgactaatattccatttgagcatgagcttccagttgcaggtcttcactggatgttcactgatttatctgggcgtagtatcgtgattgaaaatatggccgacggtatgcatattcatgaaaatccagttggtgttttaacgaatgacccaacttttgattggcacttaactaatttaagacagtatgttacccttgaaaaagcaacaccggctccaaaacaaatgggagatttactgcttcatccatatggtcatggtttaggaatgtgtggaattccaggagatgcttcacctgctgcacgatttgttagaacggtcttctttagagattcaatcgttgaagcgaatgatgaaatcagtggcgttactgctttcctcaacgtgttaacaggggtaacagttattaaagggtctgaagttgatcccgatggctcaatgaattataccgtgtataaatcagcgatgtgtcaagaaagtggaacttactattataccgattattacaatcgacgtatcaatgccgttaaattatcaaatgctaatttagactcaaaagaaattacaagtttcccttatcaaggagagcaagatatcttgttccaaaactaa

SEQ ID NO: 3 - DNA, 990 bp, Turicibacter sanguinis
  source 1..990: mol_type = genomic DNA
atgtgcacaggattatgcttatctacaaaggatggaaaacatctttttggacgaaaccttgatgttcctgcttcttataaccaggccgttcaaattgttccaaggaattttaagtggctaaacgttgcgacccaagaaacaatcacctctaaatacgcttgcatagccatgggaatcgtcattgacaatcaccctcttctctttgacggggttaatgaaaagggattagctggtggaggattaaatttcacacacttcgcaaaattctcttcaactgctgtaaaagataaaatctctttatcagcttctgatttcgtctattgggtgctgagtacgttttcatctttaagcgagttaaaagagaccttaccctctgttatcttaacaagtattcccttcaaaccggatttaccagttgcgggattacattggatttttacagataaaacaggtgaaagtattgttattgaatatatggaagacggaatgcatatccacgataatccagtcggtgttctaacaaacgacccaaccttcgactggcaattaacaaatttaagccaatatgtaaccctttcttgtaaaactccacaaccagaagaaatggggaatctactcgttaaaccatttggtcatggacttggcatgtgcggaattccaggagacggttctcccgctgcaagatttgtccgcaccgtctttttccgtgatgccgttgttggagccgacgatgaaataagtggggttacagccttctttaatgttctatcagaagttacggtgatgaaaggatctgaaattgaccccgatggatccatgaactttaccgtttataaatcagccatgtgtcaagaatctcaaacttattactacacagattactataaccgtcgaatcaacgcggttaaattaacacctgatacaatgaatgctgaccacattacaacgttcccttacctaggaaaacaagatatttgttatcaaaattaa

SEQ ID NO: 4 - DNA, 990 bp, Turicibacter sanguinis
  source 1..990: mol_type = genomic DNA
atgtgtacagcattatcattaacagcaaaagatggttcacatttattcggtagaaatatggatattgaatattcatttaatcaatcaattttattaacaccacgtcgttttgattataaaaatcgtgcaacaggtgaaatgaatcaaacaaagtatgcgattattggaatgggaaccattattgatgaacatccatgttatgcagagctttttaatgaaaaaggattagcggcagcaggattaaacttccctaactacgcacactgggatgaaaaagcaatcgaaggaaaaacaaatatccctccttatgatttagttttatgggtcacttcaaattttgaaacagttaaagaagtaaaagaagctttaaaagatgttgttttagttgatgttccagtcaatgaacaaactccaattgctcctttacactggatgatttgtgataaaacaggtgaaagtatcgttgtagaaaaaacagtaaatggtttaagtgttatggataataaagtaggagttttaacgaatgcgccaacttttgattggcatttaacaaacttaactcaatacatgggattaacgtcgacacaaccaaaagatataacactcggagaacaagaattacatccattagggcaagggcttggtgctttctcattaccaggagattattcatcaccatcacgatttgttaaagctgctttcttacgtaataatatcgattatgcaaatgttaattactctggaatcagtgaattcttccacatcttaaacggagttgcaatggttcgaggatcagtcgttactcctcaacatttaaatgacattacgttatatacatcttgtatggatcaagaacgtggaatttattattacaatacttatacaaatcatacgatttcatctattaatatgcataatgaagatttagacgcaaaagaaattaaatcatttaaatttaatgatgaatttgctgtaaatttacaaaattaa

SEQ ID NO: 5 - DNA, 990 bp, Turicibacter sanguinis
  source 1..990: mol_type = genomic DNA
atgtgtacagctattacattaaagacacaagatggattgcatgcattagggagaaatttagatattgtggctttattagatgttgctgttattttgattccgaggtcatatgcatttactcatactatcatgagtctaaaaaagaaaaataaatacgcgatggttggaatgtctacgacgtttgaaaatcatgtcttattagttgacgggatgaatgaaaagggattagcttgcgctgtattagagttacctaaatatgcttcatggagtaaagcattggataaagataaaataaatatggctccttatgattttgtttattggattttagcaaattttcaatctcttgaggaagtaaaagacggattaaaaaatgtaaatctagttaatgagtcgcttgaagggaaagaggtatcagtagatgttcattggattgtaactgatcgtacaggacagtctattgtaatagaaaaaacgaaggggaattttcgaatttataataataaggtgggagttttaactaatgctccaacattcgattggcatctcataaatttaaatcggtatatgaatatacaagtgacgaatccgcataaggtgaagtggggacatcaggagttaagttttgactcagaaggttttgggggaatcggactaccaggagatgtttcatcttcatctcgttttgtaaaagcagcttttctaagaaatcatattagagtagaaaaaggagaagatgctttaataacaagtacttttcatattttaagtaatgtggctgttatcaaaggaactgctgttacttgccaccaacaatatttgaaaacgcagtgtactagttgtatgtgtttagagacaggcgtttattactataacacttataataataatcaaattaatgctattcatttatttgacgagaatttagatgcctctgaagttaaagtgtttccttatcaagataaactggtagtacagaaacaaaattaa

SEQ ID NO: 6 - DNA, 993 bp, Turicibacter sanguinis
  source 1..993: mol_type = genomic DNA
gtgtgtacagccattacattaaaaacaagcgaaaatcatcatcttgttggacggaattttgatattcacccaatgaatgatttatcagttgctttagttccacgagaatttgaatatgtaaatcgtgtgacgaatgaagaaatgaaaacgaaatatgcagttttaggaatgggattattttatgaaaatcatattcttttttgtgatggagtaaatgaaaaaggattatcttgtctgatgttacaattatctaagttttctacttggagtcataaaatcaggaaggataaggtcaatatagccccatatgatgtagctttttgggttttatctaatttttcaacgatttctgaattaatggaaggtcttaaacagttaaatattgtggctcttcctgatgatcaaacggcattatcgacagaaattcattggcttgttagtgatacgagtggtcaatcgatcgtcattgagcgaacaagagataagttaacagtttataataataaggtaggcgttttagcaaattctccaacctttgattggcatttaaataacttagattgttatattaatgtcaaatcagagcagccagaggaaactaaatggggacaacaaatgttatcgccttattctaatggctttggtacgattggacttcctggcgatttttcttctccgtcacgatttgtaaaggctgcttttttacgaaatcatgtgaacgttggggaaggtgatgaatcagcaattagtgaatgctttcatatactagataactttgttgtaccacgtggggtggttgagactccaaaaagaaaagagtgtcatttgactaagtatagtgcatgtctttgtttagagactcaattatattattataagaccagcagtaatcagcaaattcaagtcattgatttaaacaaagaaaatttagatgctaaaggattaaaactatttccttatccaacaagattaacagtccatgatcagaattag

SEQ ID NO: 7 - DNA, 897 bp, Turicibacter sanguinis
  source 1..897: mol_type = genomic DNA
atgtgtacagcaatctcaatcaagcatcaaacagaccacgcatttttagcaagaaactttgattatgattcgaccagtcaattaaagttagctaaaattccaagaaattatagctccccgcttgaaccaacgctcaacacctttaaaacccgatattcgatcattggcatgatgctatggcatgaaggaacccatgtcctagtagatggcatgaatgaaaaggggctatcaggtgccattttaaatttacctgacgcctgcgtgtggaataaagccctcatttccgatggcatcaatctcttacctaccgatgtctccttctattgtttatcgcagttttcttctgtcaccgaattaaaggcggccattcatcaactcaatattgtggcccccgaggatcatccctttgctaaaaccactcaaattcactggatgttttgcgataaaactggtgagtcaatcgtcgtcgaacaaacggaatcaggcctatgtatttatgataaccctattggggttctaactaatgggccaaccttcgatcaacaattaatcaacatttccccctatctcaaagatagccaaacggctcctcccctcccaggagatgattcctctccctcacgctttatccgggcggggtatttaaaacaccatcttcaatggactcaacagccgctatcgacggtaactcattgctttcacattctaggaaatgtcgccctccttcctgggattttaaagcaaacacaaggagaagagtttgagacgcgttatacagcttgtatggaccttaaacacttgcgctattacctgaaatggtatcatcatctaaagactcaaatcattgacttaaaacaagagcaggatgaaagcaagacgctgactttttttgagtaa

SEQ ID NO: 8 - DNA, 741 bp, Turicibacter sanguinis
  source 1..741: mol_type = genomic DNA
gtgaaggaaggaaccatccccttacaacctatctttgccgttacttacttcttaagtatgtgtgctaccgtcgaagaagtcatttcaaaattagaaaatgatgttacacttatcgcaaagcctgtcttcggggatatccgtaatactcactggatgtttagtgaccgtacaggagaaaccatcatcattgaacctgatgtagataaactgaagattcaccgacattcaatgggcgtcttaacaaatagtccaaattacgattggcatcgtaccaatttattaaattactgcaatatacgaagtctcgattattcaagcgttacactgaatgacgatacgattgaagcctgtttctcagggagcggagcagctggactgccaggagattttagctcgccatctcgatttacacgtttagccttcctcaaaaattacgcctgcaaaggaaaaaatgaaaccgaagctgttacctatatgttccaaacatttaaaaacgttcaatttccaatggggatcgttgaagttggcgaagataaaaccattacggagcatgatagtggcgtcgttttatttgattacacgatttacactgccgttatgtgctctgaatctcttcgctattactgggtaagctaccaaaacatgcgtattcaatgtgtggatatgaacccattaattgagaaaaaacaagctgttcaattcgaattaaatcctataaatgacatcaaatatcttaattaa

SEQ ID NO: 9 - DNA, 804 bp, Turicibacter sanguinis
  source 1..804: mol_type = genomic DNA
atgcgaaaattagagaatgcaatagctcttgtcacgtcttcaacaagaggtattgggttagcgtgtgctaaaaaattagcgagtgagggtgccattgtttatatgggagttcgtcgtttagaggtcactcaagaaatttgtgatgaagtggctaaagagggtttgaaaatgaagcctgtcttttttgacgcctacaacattgattcttatgaaacgatggtagaagaagtgattagggaacaaggtaagattgatattttagttaataattttggaactggaagaccggaagtagatttagatttagttagtggagatgagaaagctttttttgatattttagaagcgaatatcggatcagtttatcgtatctctaaactggtgattcctcatatgattaaacaaggaaaaggaagcattgttaatatttcatcgattggtggaacggtcccagatatttctcgtattggttatggtgtttcaaaagcaggcgttaacaatataactcaacaaatcgctatgcagtatgcgcgctataatataagatgtaatgcggtattaccaggtttaacagcaaccgatgcagctcttgataatatgccagaacaatttattaagtctttcttatcgcatgttcctttaaatcgcatgggaacacctgaagacatggcgaatgcggttctatttttcgctagcgatgattcttcttatgtaacgggggatattatggaggtatcaggtggttatcatttaggaacaccacaatatgctgattttgttggtcgtaaagtagttgaggaaaag

SEQ ID NO: 10 - RNA, 1508 nt, Turicibacter sanguinis
  source 1..1508: mol_type = rRNA
agagtttgatcctggctcaggatgaacgctggcggcgtgcctaatacatgcaagtcgagcgaaccacttcggtggtgagcggcgaacgggtgagtaacacgtaggttatctgcccatcagacggggacaacgattggaaacgatcgctaataccggataggacgaaagtttaaaggtgcttcggcaccactgatggatgagcctgcggcgcattagctagttggtagggtaaaggcctaccaaggcgacgatgcgtagccgacctgagagggtgaacggccacactgggactgagacacggcccagactcctacgggaggcagcagtagggaatcttcggcaatgggcgaaagcctgaccgagcaacgccgcgtgaatgatgaaggccttcgggttgtaaaattctgttataagggaagaatggctctagtaggaaatggctagagtgtgacggtaccttatgagaaagccacggctaactacgtgccagcagccgcggtaatacgtaggtggcgagcgttatccggaattattgggcgtaaagagcgcgcaggtggttgattaagtctgatgtgaaagcccacggcttaaccgtggagggtcattggaaactggtcaacttgagtgcagaagagggaagtggaattccatgtgtagcggtgaaatgcgtagagatatggaggaacaccagtggcgaaggcggcttcctggtctgtaactgacactgaggcgcgaaagcgtggggagcaaacaggattagataccctggtagtccacgccgtaaacgatgagtgctaagtgttgggggtcgaacctcagtgctgaagttaacgcattaagcactccgcctggggagtacggtcgcaagactgaaactcaaaggaattgacggggacccgcacaagcggtggagcatgtggtttaattcgaagcaacgcgaagaaccttaccaggtcttgacataccagtgaccgtcctagagataggattttcccttcggggacaatggatacaggtggtgcatggttgtcgtcagctcgtgtcgtgagatgttgggttaagtcccgcaacgagcgcaacccctgtcgttagttgccagcattcagttggggactctaacgagactgccagtgacaaactggaggaaggtggggatgacgtcaaatcatcatgccccttatgacctgggctacacacgtgctacaatggttggtacaaagagaagcgaagcggtgacgtggagcaaacctcataaagccaatctcagttcggattgtaggctgcaactcgcctacatgaagttggaatcgctagtaatcgcgaatcagcatgtcgcggtgaatacgttcccgggtcttgtacacaccgcccgtcacaccacgagagtttacaacacccgaagtcagtggcctaaccgcaaggagggagctgcctaaggtggggtagatgattggggtgaagtcgtaacaaggtatccctaccggaaggtggggatggatcacctcctt

SEQ ID NO: 11 - RNA, 1509 nt, Turicibacter sanguinis
  source 1..1509: mol_type = rRNA
agagtttgatcctggctcaggatgaacgctggcggcgtgcctaatacatgcaagtcgagcgaaccacttcggtgggaagcggcgaacgggtgagtaacacgtaggtgatctgcccatcagacggggacaacgattggaaacgatcgctaataccggataggacgaaagtttaaagatgctcctggcatcactgatggatgagcctgcggcgcattagctagttggtggggtaaaggcctaccaaggcgacgatgcgtagccgacctgagagggtgaacggccacactgggactgagacacggcccagactcctacgggaggcagcagtagggaatcttcggcaatgggcgaaagcctgaccgagcaacgccgcgtgaatgaagaaggccttcgggttgtaaaattctgttataagggaagaaaggtgataggaggaaatgactatcaattgacggtaccttatgagaaagccacggctaactacgtgccagcagccgcggtaatacgtaggtggcaagcgttatccggaattattgggcgtaaagagcgcgcaggtggttaattaagtctgatgtgaaagcccacggcttaaccgtggagggtcattggaaactggttgacttgagtgcagaagagggaagtggaattccatgtgtagcggtgaaatgcgtagagatatggaggaacaccagtggcgaaggcggcttcctggtctgcaactgacactgaggcgcgaaagcgtggggagcaaacaggattagataccctggtagtccacgccgtaaacgatgagtgctaagtgttgggggtcgaacctcagtgctgaagttaacgcattaagcactccgcctggggagtacggtcgcaagactgaaactcaaaggaattgacggggacccgcacaagcggtggagcatgtggtttaattcgaagcaacgcgaagaaccttaccaggtcttgacataccattgacgcctctagagatagagggtttccttcggggacaatggatacaggtggtgcatggttgtcgtcagctcgtgtcgtgagatgttgggttaagtcccgcaacgagcgcaacccctgtcgttagttgccagcagtgagatggggactctaacgagactgccagtgacaaactggaggaaggtggggatgacgtcaaatcatcatgccccttatgacctgggctacacacgtgctacaatggttggtacaaagagaagcgaagcggtgacgtggagcaaacctcataaagccaatctcagttcggattgtaggctgcaactcgcctacatgaagttggaatcgctagtaatcgcgaatcagaatgtcgcggtgaatacgttcccgggtcttgtacacaccgcccgtcacaccacgagagtttacaacacccgaagtcagtggcctaaccgcaaggagggagctgcctaaggtggggtagatgattggggtgaagtcgtaacaaggtatccctaccggaaggtggggatggatcacctcctt

SEQ ID NO: 12 - RNA, 1508 nt, Turicibacter sanguinis; identical to SEQ ID NO: 10
  source 1..1508: mol_type = rRNA

SEQ ID NO: 13 - RNA, 1509 nt, Turicibacter sanguinis
  source 1..1509: mol_type = rRNA
agagtttgatcctggctcaggatgaacgctggcggcgtgcctaatacatgcaagtcgagcgaaccacttcggtggtgagcggcgaacgggtgagtaacacgtaggtgatctgcccatcagacggggacaacgattggaaacgatcgctaataccggataggacgaaagtttaaaggtgcttctggcaccgctgatggatgagcctgcggcgcattagctagttggtagggtaaaggcctaccaaggcgacgatgcgtagccgacctgagagggtgaacggccacactgggactgagacacggcccagactcctacgggaggcagcagtagggaatcttcggcaatgggcgaaagcctgaccgagcaacgccgcgtgaatgatgaaggccttcgggttgtaaaattctgttataagggaagaacgactttagtaggaaatggctagagtgtgacggtaccttatgagaaagccacggctaactacgtgccagcagccgcggtaatacgtaggtggcgagcgttatccggaattattgggcgtaaagagcgcgcaggtggttgattaagtctgatgtgaaagcccacggcttaaccgtggagggtcattggaaactggtcgacttgagtgcagaagagggaagtggaattccatgtgtagcggtgaaatgcgtagagatatggaggaacaccagtggcgaaggcggcttcctggtctgtaactgacactgaggcgcgaaagcgtggggagcaaacaggattagataccctggtagtccacgccgtaaacgatgagtgctaagtgttgggggtcgaacctcagtgctgaagttaacgcattaagcactccgcctggggagtacggtcgcaagactgaaactcaaaggaattgacggggacccgcacaagcggtggagcatgtggtttaattcgaagcaacgcgaagaaccttaccaggtcttgacataccattgaccgttctagagataggattttcccttcggggacaatggatacaggtggtgcatggttgtcgtcagctcgtgtcgtgagatgttgggttaagtcccgcaacgagcgcaacccctgtcgttagttgccagcattcagttggggactctaacgagactgccagtgacaaactggaggaaggtggggatgacgtcaaatcatcatgccccttatgacctgggctacacacgtgctacaatggttggtacaaagagaagcgaagcggtgacgtggagcaaacctcataaagccaatctcagttcggattgtaggctgcaactcgcctacatgaagttggaatcgctagtaatcgcgaatcagaatgtcgcggtgaatacgttcccgggtcttgtacacaccgcccgtcacaccacgagagtttacaacacccgaagtcagtggcctaaccgcaaggagggagctgcctaaggtggggtagatgattggggtgaagtcgtaacaaggtatccctaccggaaggtggggatggatcacctcctt

SEQ ID NO: 14 - RNA, 1509 nt, Turicibacter sanguinis
  source 1..1509: mol_type = rRNA
agagtttgatcctggctcaggatgaacgctggcggcgtgcctaatacatgcaagtcgagcgaaccacttcggtgggaagcggcgaacgggtgagtaacacgtaggtgatctgcccatcagacggggacaacgattggaaacgatcgctaataccggataggacgaaagtttaaagatgctcctggcatcactgatggatgagcctgcggcgcattagctagttggtggggtaaaggcctaccaaggcgacgatgcgtagccgacctgagagggtgaacggccacactgggactgagacacggcccagactcctacgggaggcagcagtagggaatcttcggcaatgggcgaaagcctgaccgagcaacgccgcgtgaatgaagaaggccttcgggttgtaaaattctgttataagggaagaaaggtgataggaggaaatgactatcaattgacggtaccttatgagaaagccacggctaactacgtgccagcagccgcggtaatacgtaggtggcaagcgttatccggaattattgggcgtaaagagcgcgcaggtggttaattaagtctgatgtgaaagcccacggcttaaccgtggagggtcattggaaactggttgacttgagtgcagaagagggaagtggaattccatgtgtagcggtgaaatgcgtagagatatggaggaacaccagtggcgaaggcggcttcctggtctgcaactgacactgaggcgcgaaagcgtggggagcaaacaggattagataccctggtagtccacgccgtaaacgatgagtgctaagtgttgggggtcgaacctcagtgctgaagttaacgcattaagcactccgcctggggagtacggtcgcaagactgaaactcaaaggaattgacggggacccgcacaagcggtggagcatgtggtttaattcgaagcaacgcgaagaaccttaccaggtcttgacataccattgacgcctctagagatagagggtttccttcggggacaatggatacaggtggtgcatggttgtcgtcagctcgtgtcgtgagatgttgggttaagtcccgcaacgagcgcaacccctgtcgttagttgccagcagtaagatggggactctaacgagactgccagtgacaaactggaggaaggtggggatgacgtcaaatcatcatgccccttatgacctgggctacacacgtgctacaatggttggtacaaagagaagcgaagcggtgacgtggagcaaacctcataaagccaatctcagttcggattgtaggctgcaactcgcctacatgaagttggaatcgctagtaatcgcgaatcagaatgtcgcggtgaatacgttcccgggtcttgtacacaccgcccgtcacaccacgagagtttacaacacccgaagtcagtggcctaaccgcaaggagggagctgcctaaggtggggtagatgattggggtgaagtcgtaacaaggtatccctaccggaaggtggggatggatcacctcctt

SEQ ID NO: 15 - RNA, 1508 nt, Turicibacter sanguinis; identical to SEQ ID NO: 10
  source 1..1508: mol_type = rRNA

SEQ ID NO: 16 - RNA, 1509 nt, Turicibacter sanguinis; identical to SEQ ID NO: 13
  source 1..1509: mol_type = rRNA

SEQ ID NO: 17 - RNA, 1474 nt, Turicibacter sanguinis
  source 1..1474: mol_type = rRNA
agagtttgatcatggctcaggatgaacgctggcggcgtgcctaatacatgcaagtcgagcgaaccacttcggtggtgagcggcgaacgggtgagtaacacgtaggttatctgcccatcagacggggacaacgattggaaacgatcgctaataccggataggacgaaagtttaaaggtgcttcggcaccactgatggatgagcctgcggcgcattagctagttggtagggtaaaggcctaccaaggcgacgatgcgtagccgacctgagagggtgaacggccacactgggactgagacacggcccagactcctacgggaggcagcagtagggaatcttcggcaatgggcgaaagcctgaccgagcaacgccgcgtgaatgatgaaggccttcgggttgtaaaattctgttataagggaagaatggctctagtaggaaatggctagagtgtgacggtaccttatgagaaagccacggctaactacgtgccagcagccgcggtaatacgtaggtggcgagcgttatccggaattattgggcgtaaagagcgcgcaggtggttgattaagtctgatgtgaaagcccacggcttaaccgtggagggtcattggaaactggtcaacttgagtgcagaagagggaagtggaattccatgtgtagcggtgaaatgcgtagagatatggaggaacaccagtggcgaaggcggcttcctggtctgtaactgacactgaggcgcgaaagcgtggggagcaaacaggattagataccctggtagtccacgccgtaaacgatgagtgctaagtgttgggggtcgaacctcagtgctgaagttaacgcattaagcactccgcctggggagtacggtcgcaagactgaaactcaaaggaattgacggggacccgcacaagcggtggagcatgtggtttaattcgaagcaacgcgaagaaccttaccaggtcttgacataccagtgaccgtcctagagataggattttcccttcggggacaatggatacaggtggtgcatggttgtcgtcagctcgtgtcgtgagatgttgggttaagtcccgcaacgagcgcaacccctgtcgttagttgccagcattcagttggggactctaacgagactgccagtgacaaactggaggaaggtggggatgacgtcaaatcatcatgccccttatgacctgggctacacacgtgctacaatggttggtacaaagagaagcgaagcggtgacgtggagcaaacctcataaagccaatctcagttcggattgtaggctgcaactcgcctacatgaagttggaatcgctagtaatcgcgaatcagcatgtcgcggtgaatacgttcccgggtcttgtacacaccgcccgtcacaccacgagagtttacaacacccgaagtcagtggcctaaccgcaaggagggagctgcctaaggtggggtagatgattggggtgaagtcgtaacaaggta

SEQ ID NO: 18 - RNA, 1475 nt, Turicibacter sanguinis
  source 1..1475: mol_type = rRNA
agagtttgatcatggctcaggatgaacgctggcggcgtgcctaatacatgcaagtcgagcgaaccacttcggtggtgagcggcgaacgggtgagtaacacgtaggtgatctgcccatcagacggggacaacgattggaaacgatcgctaataccggataggacgaaagtttaaaggtgcttctggcaccgctgatggatgagcctgcggcgcattagctagttggtagggtaaaggcctaccaaggcgacgatgcgtagccgacctgagagggtgaacggccacactgggactgagacacggcccagactcctacgggaggcagcagtagggaatcttcggcaatgggcgaaagcctgaccgagcaacgccgcgtgaatgatgaaggccttcgggttgtaaaattctgttataagggaagaacgactttagtaggaaatggctagagtgtgacggtaccttatgagaaagccacggctaactacgtgccagcagccgcggtaatacgtaggtggcgagcgttatccggaattattgggcgtaaagagcgcgcaggtggttgattaagtctgatgtgaaagcccacggcttaaccgtggagggtcattggaaactggtcgacttgagtgcagaagagggaagtggaattccatgtgtagcggtgaaatgcgtagagatatggaggaacaccagtggcgaaggcggcttcctggtctgtaactgacactgaggcgcgaaagcgtggggagcaaacaggattagataccctggtagtccacgccgtaaacgatgagtgctaagtgttgggggtcgaacctcagtgctgaagttaacgcattaagcactccgcctggggagtacggtcgcaagactgaaactcaaaggaattgacggggacccgcacaagcggtggagcatgtggtttaattcgaagcaacgcgaagaaccttaccaggtcttgacataccattgaccgttctagagataggattttcccttcggggacaatggatacaggtggtgcatggttgtcgtcagctcgtgtcgtgagatgttgggttaagtcccgcaacgagcgcaacccctgtcgttagttgccagcattcagttggggactctaacgagactgccagtgacaaactggaggaaggtggggatgacgtcaaatcatcatgccccttatgacctgggctacacacgtgctacaatggttggtacaaagagaagcgaagcggtgacgtggagcaaacctcataaagccaatctcagttcggattgtaggctgcaactcgcctacatgaagttggaatcgctagtaatcgcgaatcagaatgtcgcggtgaatacgttcccgggtcttgtacacaccgcccgtcacaccacgagagtttacaacacccgaagtcagtggcctaaccgcaaggagggagctgcctaaggtggggtagatgattggggtgaagtcgtaacaaggta

SEQ ID NO: 19 - DNA, 23 bp, synthetic construct
  source 1..23: mol_type = other DNA
ggtttagctgatgctggaattag

SEQ ID NO: 20 - DNA, 30 bp, synthetic construct
  source 1..30: mol_type = other DNA
tccaaatttatcaacaaatgctgtaataat

SEQ ID NO: 21 - DNA, 17 bp, synthetic construct
  source 1..17: mol_type = other DNA
gggtttgcagatgcggg

SEQ ID NO: 22 - DNA, 26 bp, synthetic construct
  source 1..26: mol_type = other DNA
aatttatcaaccaccgctgtaataat

SEQ ID NO: 23 - DNA, 17 bp, synthetic construct
  source 1..17: mol_type = other DNA
gggtttgctgatgccgc

SEQ ID NO: 24 - DNA, 30 bp, synthetic construct
  source 1..30: mol_type = other DNA
cccaaatttatcaactactgctgtaataat

SEQ ID NO: 25 - DNA, 18 bp, synthetic construct
  source 1..18: mol_type = other DNA
ctcgagcaccaccaccac

SEQ ID NO: 26 - DNA, 28 bp, synthetic construct
  source 1..28: mol_type = other DNA
gaatacctccttaagttgttctagaagg

SEQ ID NO: 27 - DNA, 38 bp, synthetic construct
  source 1..38: mol_type = other DNA
aacaacttaaggaggtattcatgtgtacaggacttagc

SEQ ID NO: 28 - DNA, 50 bp, synthetic construct
  source 1..50: mol_type = other DNA
tggtggtggtggtgctcgaggttttggaataaaatatcttgtttaccttg

SEQ ID NO: 29 - DNA, 43 bp, synthetic construct
  source 1..43: mol_type = other DNA
aacaacttaaggaggtattcatgtgtacagggttaagtttaac

SEQ ID NO: 30 - DNA, 47 bp, synthetic construct
  source 1..47: mol_type = other DNA
tggtggtggtggtgctcgaggttttggaacaagatatcttgctctcc

SEQ ID NO: 31 - DNA, 38 bp, synthetic construct
  source 1..38: mol_type = other DNA
aacaacttaaggaggtattcatgtgcacaggattatgc

SEQ ID NO: 32 - DNA, 51 bp, synthetic construct
  source 1..51: mol_type = other DNA
tggtggtggtggtgctcgagattttgataacaaatatcttgttttcctagg

SEQ ID NO: 33 - DNA, 43 bp, synthetic construct
  source 1..43: mol_type = other DNA
aacaacttaaggaggtattcatgtgtacagcattatcattaac

SEQ ID NO: 34 - DNA, 51 bp, synthetic construct
  source 1..51: mol_type = other DNA
tggtggtggtggtgctcgagattttgtaaatttacagcaaattcatcatta

SEQ ID NO: 35 - DNA, 44 bp, synthetic construct
  source 1..44: mol_type = other DNA
aacaacttaaggaggtattcatgtgtacagctattacattaaag

SEQ ID NO: 36 - DNA, 48 bp, synthetic construct
  source 1..48: mol_type = other DNA
tggtggtggtggtgctcgagattttgtttctgtactaccagtttatct

SEQ ID NO: 37 - DNA, 46 bp, synthetic construct
  source 1..46: mol_type = other DNA
aacaacttaaggaggtattcatgtgtacagccattacattaaaaac

SEQ ID NO: 38 - DNA, 47 bp, synthetic construct
  source 1..47: mol_type = other DNA
tggtggtggtggtgctcgagattctgatcatggactgttaatcttgt

SEQ ID NO: 39 - DNA, 41 bp, synthetic construct
  source 1..41: mol_type = other DNA
aacaacttaaggaggtattcatgtgtacagcaatctcaatc

SEQ ID NO: 40 - DNA, 43 bp, synthetic construct
  source 1..43: mol_type = other DNA
tggtggtggtggtgctcgagctcaaaaaaagtcagcgtcttgc

SEQ ID NO: 41 - DNA, 38 bp, synthetic construct
  source 1..38: mol_type = other DNA
aacaacttaaggaggtattcatgaaggaaggaaccatc

SEQ ID NO: 42 - DNA, 57 bp, synthetic construct
  source 1..57: mol_type = other DNA
tggtggtggtggtgctcgagattaagatatttgatgtcatttataggatttaattcg

SEQ ID NO: 43 - DNA, 46 bp, synthetic construct
  source 1..46: mol_type = other DNA
aacaacttaaggaggtattcatgcgaaaattagagaatgcaatagc

SEQ ID NO: 44 - DNA, 44 bp, synthetic construct
  source 1..44: mol_type = other DNA
tggtggtggtggtgctcgagcttttcctcaactactttacgacc

SEQ ID NO: 45 - DNA, 18 bp, synthetic construct
  source 1..18: mol_type = other DNA
ggatctgattacaaggac

SEQ ID NO: 46 - DNA, 29 bp, synthetic construct
  source 1..29: mol_type = other DNA
tattttaaatttttttgaatccatagatc

SEQ ID NO: 47 - DNA, 46 bp, synthetic construct
  source 1..46: mol_type = other DNA
tggattcaaaaaaatttaaaataatgtgtacagcattatcattaac

SEQ ID NO: 48 - DNA, 44 bp, synthetic construct
  source 1..44: mol_type = other DNA
tcgtccttgtaatcagatccattttgtaaatttacagcaaattc

SEQ ID NO: 49 - DNA, 41 bp, synthetic construct
  source 1..41: mol_type = other DNA
tggattcaaaaaaatttaaaataatgtgtacaggacttagc

SEQ ID NO: 50 - DNA, 46 bp, synthetic construct
  source 1..46: mol_type = other DNA
tcgtccttgtaatcagatccgttttggaataaaatatcttgtttac

SEQ ID NO: 51 - DNA, 41 bp, synthetic construct
  source 1..41: mol_type = other DNA
tggattcaaaaaaatttaaaataatgtgcacaggattatgc

SEQ ID NO: 52 - DNA, 46 bp, synthetic construct
  source 1..46: mol_type = other DNA
tcgtccttgtaatcagatccattttgataacaaatatcttgttttc

SEQ ID NO: 53 - DNA, 46 bp, synthetic construct
  source 1..46: mol_type = other DNA
tggattcaaaaaaatttaaaataatgtgtacagggttaagtttaac

SEQ ID NO: 54 - DNA, 41 bp, synthetic construct
  source 1..41: mol_type = other DNA
tcgtccttgtaatcagatccgttttggaacaagatatcttg

SEQ ID NO: 55 - DNA, 21 bp, synthetic construct
  source 1..21: mol_type = other DNA
cagtgctcagagctcgaaagt

SEQ ID NO: 56 - DNA, 20 bp, synthetic construct
  source 1..20: mol_type = other DNA
gtgttcttcgacatcacggc

SEQ ID NO: 57 - DNA, 20 bp, synthetic construct
  source 1..20: mol_type = other DNA
tccagggtttcagacactgg

SEQ ID NO: 58 - DNA, 19 bp, synthetic construct
  source 1..19: mol_type = other DNA
gccgaacgaagaaacatgg

SEQ ID NO: 59 - DNA, 26 bp, synthetic construct
  source 1..26: mol_type = other DNA
agcaactaaacaacctgccagtacta

SEQ ID NO: 60 - DNA, 21 bp, synthetic construct
  source 1..21: mol_type = other DNA
gtccggatattcaaggatgca